SEQUENCE LISTING (183 sequences)

SEQ ID NO: 1 - protein, 227 aa, synthetic construct
  REGION 1..227: note = Pertuzumab heavy chain Fab (Domain boundaries: VH; E1 - S119, CH1; A120 - V217, Hinge (partial); E218 - T227)
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 2 - protein, 214 aa, synthetic construct
  REGION 1..214: note = Pertuzumab light chain (kappa) (Domain boundaries: VL; D1 - K107, CL; R108 - C214)
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 226 aa, synthetic construct
  REGION 1..226: note = CAT-2200 heavy chain Fab (Domain boundaries: VH; E1 - S118, CH1; A119 - V216, Hinge(partial); E217 - T226)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLIHGVTRNWGQGTLVTVSSASTKGPSVFPLAPSSKSTSGGTAALGCLVKDYFPQPVTVSWNSGALTSGVHTFPAVLQSSGLYSLSSVVTVPSSSLGTQTYICNVNHKPSNTKVDKKVEPKSCDKTHT

SEQ ID NO: 4 - protein, 216 aa, synthetic construct
  REGION 1..216: note = CAT-2200 light chain (lambda) (Domain boundaries: VL; N1 - L110, CL; G111 - S216)
NFMLTQPHSVSESPGKTVTISCTRSSGSLANYYVQWYQQRPGSSPTIVIFANNQRPSGVPDRFSGSIDSSSNSASLTISGLKTEDEADYYCQTYDPYSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 222 aa, synthetic construct
  REGION 1..222: note = IgG1 Fc (Domain boundaries: Hinge (partial); C1 - P5, CH2; A6 - K115, CH3; G116 - K222)
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 666 bp, synthetic construct
  misc_feature 1..666: note = synthetic IgG1 Fc
  source 1..666: mol_type = other DNA
tgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaa

SEQ ID NO: 7 - DNA, 681 bp, synthetic construct
  misc_feature 1..681: note = Pertuzumab heavy chain Fab
  source 1..681: mol_type = other DNA
gaagtgcagctggtcgaatctggaggaggactggtgcagccaggagggtccctgcgcctgtcttgcgccgctagtggcttcacttttaccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctccgcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtggaacccaagtcttgtgataaaacccataca

SEQ ID NO: 8 - DNA, 642 bp, synthetic construct
  misc_feature 1..642: note = Pertuzumab light chain (kappa)
  source 1..642: mol_type = other DNA
gatattcagatgacccagtccccaagctccctgagtgcctcagtgggcgaccgagtcaccatcacatgcaaggcttcccaggatgtgtctattggagtcgcatggtaccagcagaagccaggcaaagcacccaagctgctgatctatagcgcctcctaccggtataccggcgtgccctctagattctctggcagtgggtcaggaacagactttactctgaccatctctagtctgcagcctgaggatttcgctacctactattgccagcagtactatatctacccatatacctttggccaggggacaaaagtggagatcaagaggactgtggccgctccctccgtcttcatttttcccccttctgacgaacagctgaaaagtggcacagccagcgtggtctgtctgctgaacaatttctaccctcgcgaagccaaagtgcagtggaaggtcgataacgctctgcagagcggcaacagccaggagtctgtgactgaacaggacagtaaagattcaacctatagcctgtcaagcacactgactctgagcaaggcagactacgagaagcacaaagtgtatgcctgcgaagtcacacatcaggggctgtcctctcctgtgactaagagctttaacagaggagagtgt

SEQ ID NO: 9 - DNA, 678 bp, synthetic construct
  misc_feature 1..678: note = CAT-2200 heavy chain Fab
  source 1..678: mol_type = other DNA
gaggtgcagctgctggaatctggggggggcctggtgcagcctggggggtccctgagactgtcatgtgctgccagcgggtttactttcagctcctacgctatgtcctgggtgcgacaggcacccgggaagggactggagtgggtctctgcaatcagtgggtcaggcgggagtacttactatgccgacagcgtgaagggacggttcactatctcaagagataacagcaagaacaccctgtatctgcagatgaacagcctgagagcagaagacacagccgtgtactattgcgccagggatctgatccacggagtcactcgcaattggggccaggggactctggtgaccgtctctagtgctagcacaaaggggccctctgtgtttccactggccccctcaagcaaaagcacatccggaggaactgcagctctgggatgtctggtgaaggactacttcccccagcctgtgaccgtctcttggaacagtggagccctgaccagcggcgtgcacacatttcctgctgtcctgcagtcctctggcctgtactccctgagttcagtggtcacagtgcctagctcctctctggggacccagacatatatttgcaacgtgaatcataaaccaagcaacactaaggtcgacaagaaagtggagcccaagagctgtgataaaactcatacc

SEQ ID NO: 10 - DNA, 648 bp, synthetic construct
  misc_feature 1..648: note = CAT-2200 light chain (lambda)
  source 1..648: mol_type = other DNA
aactttatgctgactcagccccactccgtgtccgagagccctggcaaaactgtgactatttcatgtacccgatcatctggaagcctggccaactactatgtgcagtggtaccagcagaggccaggcagctcccccactatcgtgattttcgctaacaatcagcggccttccggcgtcccagacagattttccgggtctatcgattctagttcaaatagtgcatcactgactatttccgggctgaagaccgaggacgaagccgattactattgccagacctacgacccctattctgtggtcttcggcgggggaaccaagctgacagtgctgggacagccaaaagcggcgcccagtgtcacactgtttccccctagctccgaggaactgcaggctaacaaagcaacactggtgtgtctgatcagcgacttctaccctggagctgtgactgtcgcctggaaggctgattctagtccagtgaaagcaggcgtcgagaccacaactccctctaagcagagtaacaacaagtacgcagcctcaagctatctgtcactgaccccagaacagtggaagagccaccggagctattcctgccaggtcactcacgaaggctccactgtcgagaaaaccgtcgctcccaccgaatgttca

SEQ ID NO: 11 - protein, 217 aa, Homo sapiens
  REGION 1..217: note = Human IgG1 Fc sequence 231-447 (EU-numbering), without hinge
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 228 aa, synthetic construct
  REGION 1..228: note = SGN-CD19a heavy chain Fab (Domain boundaries: VH; Q1-S120, CH1; A121-V218, Hinge (partial); E219-T228)
QVTLRESGPALVKPTQTLTLTCTFSGFSLSTSGMGVGWIRQPPGKALEWLAHIWWDDDKRYNPALKSRLTISKDTSKNQVVLTMTNMDPVDTAAYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 13 - protein, 213 aa, synthetic construct
  REGION 1..213: note = SGN-CD19a light chain (kappa) (Domain boundaries: VL; E1-K106, CL; R107-C213)
EIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 233 aa, synthetic construct
  REGION 1..233: note = CR8071 heavy chain Fab (Domain boundaries: VH; Q1-S125, CH1; A126-V223, Hinge (partial); E224-T233)
QVQLVQSGAEVKKPGASVRVSCRASGYIFTESGITWVRQAPGQGLEWMGWISGYSGDTKYAQKLQGRVTMTKDTSTTTAYMELRSLRYDDTAVYYCARDVQYSGSYLGAYYFDYWSPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 15 - protein, 216 aa, synthetic construct
  REGION 1..216: note = CR8071 light chain (lambda) (Domain boundaries: VL; Q1-L110, CL; R111-S216)
QSVLTQPPSASGTPGQRVTISCSGSSSNIGTNYVYWYQQFPGTAPKLLIYRSYQRPSGVPDRFSGSKSGSSASLAISGLQSEDEADYYCATWDDSLDGWVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - DNA, 684 bp, synthetic construct
  misc_feature 1..684: note = SGN-CD19a heavy chain Fab
  source 1..684: mol_type = other DNA
caggtgacactgagagaatccggcccagccctggtgaagcccactcagaccctgacactgacttgcaccttctctgggttttccctgtctacaagtgggatgggagtgggatggatcaggcagccacctggaaaagccctggagtggctggctcacatttggtgggacgatgacaagcggtacaacccagcactgaaaagcagactgacaatcagcaaggatacttccaaaaaccaggtggtcctgacaatgactaatatggaccccgtggacacagccgcttactattgcgcccgcatggaactgtggagctactatttcgactactgggggcagggaacactggtcactgtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacc

SEQ ID NO: 17 - DNA, 639 bp, synthetic construct
  misc_feature 1..639: note = SGN-CD19a light chain (kappa)
  source 1..639: mol_type = other DNA
gagatcgtgctgacccagtctccagccacactgtctctgagcccaggagagagggccaccctgtcctgctctgccagctccagcgtgagctacatgcactggtatcagcagaagccaggacaggcccctaggctgctgatctacgacaccagcaagctggcctccggcatccccgcaagattcagcggctccggctctggcacagactttaccctgacaatcagctccctggagcctgaggatttcgccgtgtactattgttttcagggcagcgtgtatccattcacctttggccagggcacaaagctggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 18 - DNA, 699 bp, synthetic construct
  misc_feature 1..699: note = CR8071 heavy chain Fab
  source 1..699: mol_type = other DNA
caggtgcagctggtccagtccggggctgaagtgaaaaaacctggggcatccgtgcgggtgtcatgtcgggcaagcgggtatatctttactgagtctggaatcacctgggtgaggcaggctcccggacagggactggaatggatgggatggatttctggatacagtggcgacacaaagtatgcacagaaactgcagggccgcgtcaccatgacaaaggatacttcaaccacaactgcctacatggagctgcggagcctgagatatgacgatacagccgtgtactattgcgcccgggacgtgcagtacagcgggtcctacctgggggcatactacttcgattactggtcacctggaactctggtcaccgtctcttcagctagcaccaagggcccttctgtgtttccactggcaccctcaagcaaaagcacctccggaggaacagcagcactgggatgtctggtcaaggactatttccccgagcctgtgaccgtctcatggaatagcggcgcactgactagtggggtgcacacctttcccgccgtcctgcagtcctctgggctgtacagcctgagttcagtggtcacagtgccaagctcctctctgggaactcagacctatatctgcaacgtcaatcataaacccagcaacacaaaggtcgacaagaaagtggagcccaagagctgtgataaaactcatacc

SEQ ID NO: 19 - DNA, 648 bp, synthetic construct
  misc_feature 1..648: note = CR8071 light chain (lambda)
  source 1..648: mol_type = other DNA
cagagcgtcctgactcagcctccctccgcctccggaacacctgggcagagagtgactatctcctgtagcggatcaagctcaaacattggaaccaactacgtgtattggtaccagcagttccccggcacagctcctaagctgctgatctatcggagctaccagagaccaagcggggtccccgacaggttttctggcagtaaatcagggagctccgccagcctggctatttccggcctgcagtctgaggacgaagcagattactattgcgccacctgggacgattccctggatggatgggtcttcggcggcggcacaaaactgaccgtcctgaggcagccaaaggcggcgcccagtgtcacactgtttccccctagctccgaggaactgcaggctaacaaagcaacactggtgtgtctgatcagcgacttctaccctggagctgtgactgtcgcctggaaggctgattctagtccagtgaaagcaggcgtcgagaccacaactccctctaagcagagtaacaacaagtacgcagcctcaagctatctgtcactgaccccagaacagtggaagagccaccggagctattcctgccaggtcactcacgaaggctccactgtcgagaaaaccgtcgctcccaccgaatgttca

SEQ ID NO: 20 - protein, 119 aa, synthetic construct [sequence omitted]

SEQ ID NO: 21 - protein, 117 aa, synthetic construct
  source 1..117: note = D3H44
EVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSS

SEQ ID NO: 22 - protein, 97 aa, synthetic construct
  source 1..97: note = X92218|IGHV3-66*01
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 23 - protein, 98 aa, synthetic construct
  source 1..98: note = M99641|IGHV1-18*01
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 24 - protein, 98 aa, synthetic construct; identical to SEQ ID NO: 23
  source 1..98: note = X62106|IGHV1-2*02

SEQ ID NO: 25 - protein, 98 aa, synthetic construct
  source 1..98: note = M99642|IGHV1-24*01
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 26 - protein, 98 aa, synthetic construct
  source 1..98: note = X62109|IGHV1-3*01
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 27 - protein, 98 aa, synthetic construct
  source 1..98: note = X92209|IGHV1-45*01
QMQLVQSGAEVKKTGSSVKVSCKASGYTFTYRYLHWVRQAPGQALEWMGWITPFNGNTNYAQKFQDRVTITRDRSMSTAYMELSSLRSEDTAMYYCAR

SEQ ID NO: 28 - protein, 98 aa, synthetic construct
  source 1..98: note = X92343|IGHV1-46*01
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 29 - protein, 98 aa, synthetic construct
  source 1..98: note = M29809|IGHV1-58*01
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 30 - protein, 98 aa, synthetic construct
  source 1..98: note = KC713934|IGHV1-69D*01, L22582|IGHV1-69*01
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 31 - protein, 98 aa, synthetic construct
  source 1..98: note = KF698734|IGHV1-69-2*01
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 32 - protein, 98 aa, synthetic construct
  source 1..98: note = M99637|IGHV1-8*01
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 33 - protein, 99 aa, synthetic construct
  source 1..99: note = M99648|IGHV2-26*01
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCAR

SEQ ID NO: 34 - protein, 99 aa, synthetic construct
  source 1..99: note = X62111|IGHV2-5*01
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAH

SEQ ID NO: 35 - protein, 99 aa, synthetic construct
  source 1..99: note = L21969|IGHV2-70*01
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCAR

SEQ ID NO: 36 - protein, 99 aa, synthetic construct
  source 1..99: note = KC713935|IGHV2-70D*04
QVTLKESGPALVKPTQTLTLTCTFSGFSLSTSGMRVSWIRQPPGKALEWLARIDWDDDKFYSTSLKTRLTISKDTSKNQVVLTMTNMDPVDTATYYCAR

SEQ ID NO: 37 - protein, 98 aa, synthetic construct
  source 1..98: note = X92287|IGHV3-11*03
QVQLLESGGGLVKPGGSLRLSCAASGFTFSDYYMSWIRQAPGKGLEWVSYISSSSSYTNYADSVKGRFTISRDNAKNSLYLQMNSLRAEDTAVYYCAR

SEQ ID NO: 38 - protein, 97 aa, synthetic construct
  source 1..97: note = X92217|IGHV3-13*01
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCAR

SEQ ID NO: 39 - protein, 100 aa, synthetic construct
  source 1..100: note = X92216|IGHV3-15*01
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 40 - protein, 98 aa, synthetic construct
  source 1..98: note = M99657|IGHV3-20*01
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCAR

SEQ ID NO: 41 - protein, 98 aa, synthetic construct
  source 1..98: note = AB019439|IGHV3-21*01
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 42 - protein, 98 aa, synthetic construct
  source 1..98: note = M99660|IGHV3-23*01
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 43 - protein, 98 aa, synthetic construct
  source 1..98: note = M83134|IGHV3-30*01
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 44 - protein, 98 aa, synthetic construct
  source 1..98: note = AC244456|IGHV3-30-5*01
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 45 - protein, 98 aa, synthetic construct
  source 1..98: note = AB019439|IGHV3-33*01
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 46 - protein, 98 aa, synthetic construct
  source 1..98: note = M99672|IGHV3-43*01
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAK

SEQ ID NO: 47 - protein, 98 aa, synthetic construct
  source 1..98: note = KC713950|IGHV3-43D*01
EVQLVESGGVVVQPGGSLRLSCAASGFTFDDYAMHWVRQAPGKGLEWVSLISWDGGSTYYADSVKGRFTISRDNSKNSLYLQMNSLRAEDTALYYCAK

SEQ ID NO: 48 - protein, 98 aa, synthetic construct
  source 1..98: note = M99675|IGHV3-48*01
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAR

SEQ ID NO: 49 - protein, 100 aa, synthetic construct
  source 1..100: note = AB019438|IGHV3-49*03
EVQLVESGGGLVQPGRSLRLSCTASGFTFGDYAMSWFRQAPGKGLEWVGFIRSKAYGGTTEYAASVKGRFTISRDDSKSIAYLQMNSLKTEDTAVYYCTR

SEQ ID NO: 50 - protein, 97 aa, synthetic construct
  source 1..97: note = M99679|IGHV3-53*01
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 51 - protein, 98 aa, synthetic construct
  source 1..98: note = M99682|IGHV3-64*01
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCAR

SEQ ID NO: 52 - protein, 98 aa, synthetic construct
  source 1..98: note = M99649|IGHV3-7*01
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 53 - protein, 100 aa, synthetic construct
  source 1..100: note = X92206|IGHV3-72*01
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAR

SEQ ID NO: 54 - protein, 100 aa, synthetic construct
  source 1..100: note = X70197|IGHV3-73*01
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTR

SEQ ID NO: 55 - protein, 98 aa, synthetic construct
  source 1..98: note = L33851|IGHV3-74*01
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAR

SEQ ID NO: 56 - protein, 98 aa, synthetic construct
  source 1..98: note = M99651|IGHV3-9*01
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAK

SEQ ID NO: 57 - protein, 98 aa, synthetic construct
  source 1..98: note = HM855939|IGHV3-NL1*01
QVQLVESGGGVVQPGGSLRLSCAASGFTFSSYGMHWVRQAPGKGLEWVSVIYSGGSSTYYADSVKGRFTISRDNSKNTLYLQMNSLRAEDTAVYYCAK

SEQ ID NO: 58 - protein, 98 aa, synthetic construct
  source 1..98: note = X05714|IGHV4-28*01
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCAR

SEQ ID NO: 59 - protein, 99 aa, synthetic construct
  source 1..99: note = L10089|IGHV4-30-2*01
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCAR

SEQ ID NO: 60 - protein, 99 aa, synthetic construct
  source 1..99: note = Z14238|IGHV4-30-4*01
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAR

SEQ ID NO: 61 - protein, 99 aa, synthetic construct
  source 1..99: note = L10098|IGHV4-31*01
QVQLQESGPGLVKPSQTLSLTCTVSGGSISSGGYYWSWIRQHPGKGLEWIGYIYYSGSTYYNPSLKSLVTISVDTSKNQFSLKLSSVTAADTAVYYCAR

SEQ ID NO: 62 - protein, 97 aa, synthetic construct
  source 1..97: note = AB019439|IGHV4-34*01
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 63 - protein, 98 aa, synthetic construct
  source 1..98: note = Z12367|IGHV4-38-2*01
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAR

SEQ ID NO: 64 - protein, 99 aa, synthetic construct
  source 1..99: note = AB019439|IGHV4-39*01
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 65 - protein, 98 aa, synthetic construct
  source 1..98: note = X05713|IGHV4-4*01
QVQLQESGPGLVKPPGTLSLTCAVSGGSISSSNWWSWVRQPPGKGLEWIGEIYHSGSTNYNPSLKSRVTISVDKSKNQFSLKLSSVTAADTAVYCCAR

SEQ ID NO: 66 - protein, 97 aa, synthetic construct
  source 1..97: note = AB019438|IGHV4-59*01
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 67 - protein, 99 aa, synthetic construct
  source 1..99: note = M29811|IGHV4-61*01
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAR

SEQ ID NO: 68 - protein, 98 aa, synthetic construct
  source 1..98: note = X92227|IGHV5-10-1*01
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCAR

SEQ ID NO: 69 - protein, 98 aa, synthetic construct
  source 1..98: note = M99686|IGHV5-51*01
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 70 - protein, 101 aa, synthetic construct
  source 1..101: note = J04097|IGHV6-1*01
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAR

SEQ ID NO: 71 - protein, 98 aa, synthetic construct
  source 1..98: note = X62110|IGHV7-4-1*02
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCAR

SEQ ID NO: 72 - protein, 17 aa, synthetic construct
  source 1..17: note = J00256|IGHJ1*01
AEYFQHWGQGTLVTVSS

SEQ ID NO: 73 - protein, 17 aa, synthetic construct
  source 1..17: note = J00256|IGHJ2*01
YWYFDLWGRGTLVTVSS

SEQ ID NO: 74 - protein, 16 aa, synthetic construct
  source 1..16: note = J00256|IGHJ3*01
DAFDVWGQGTMVTVSS

SEQ ID NO: 75 - protein, 16 aa, synthetic construct
  source 1..16: note = J00256|IGHJ5*01
NWFDSWGQGTLVTVSS

SEQ ID NO: 76 - protein, 20 aa, synthetic construct
  source 1..20: note = J00256|IGHJ6*01
YYYYYGMDVWGQGTTVTVSS

SEQ ID NO: 77 - protein, 15 aa, synthetic construct
  source 1..15: note = J00256|IGHJ4*01
YFDYWGQGTLVTVSS

SEQ ID NO: 78 - protein, 107 aa, synthetic construct [sequence omitted]

SEQ ID NO: 79 - protein, 106 aa, synthetic construct
  source 1..106: note = D3H44
DIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEI

SEQ ID NO: 80 - protein, 95 aa, synthetic construct
  source 1..95: note = Y14865|IGKV1-NL1*01
DIQMTQSPSSLSASVGDRVTITCRASQGISNSLAWYQQKPGKAPKLLLYAASRLESGVPSRFSGSGSGTDYTLTISSLQPEDFATYYCQQYYSTP

SEQ ID NO: 81 - protein, 95 aa, synthetic construct
  source 1..95: note = X59312|IGKV1D-39*01
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 82 - protein, 95 aa, synthetic construct
  source 1..95: note = X17263|IGKV1D-12*01, V01577|IGKV1-12*01
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 83 - protein, 95 aa, synthetic construct
  source 1..95: note = Z00006|IGKV1-13*02
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 84 - protein, 95 aa, synthetic construct
  source 1..95: note = J00248|IGKV1-16*01
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

SEQ ID NO: 85 - protein, 95 aa, synthetic construct
  source 1..95: note = X72808|IGKV1-17*01
DIQMTQSPSSLSASVGDRVTITCRASQGIRNDLGWYQQKPGKAPKRLIYAASSLQSGVPSRFSGSGSGTEFTLTISSLQPEDFATYYCLQHNSYP

SEQ ID NO: 86 - protein, 95 aa, synthetic construct
  source 1..95: note = X63398|IGKV1-27*01
DIQMTQSPSSLSASVGDRVTITCRASQGISNYLAWYQQKPGKVPKLLIYAASTLQSGVPSRFSGSGSGTDFTLTISSLQPEDVATYYCQKYNSAP

SEQ ID NO: 87 - protein, 95 aa, synthetic construct
  source 1..95: note = M64856|IGKV1-33*01
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 88 - protein, 95 aa, synthetic construct
  source 1..95: note = Z00001|IGKV1-5*01
DIQMTQSPSTLSASVGDRVTITCRASQSISSWLAWYQQKPGKAPKLLIYDASSLESGVPSRFSGSGSGTEFTLTISSLQPDDFATYYCQQYNSYS

SEQ ID NO: 89 - protein, 95 aa, synthetic construct
  source 1..95: note = M64858|IGKV1-6*01
AIQMTQSPSSLSASVGDRVTITCRASQGIRNDLGWYQQKPGKAPKLLIYAASSLQSGVPSRFSGSGSGTDFTLTISSLQPEDFATYYCLQDYNYP

SEQ ID NO: 90 - protein, 95 aa, synthetic construct
  source 1..95: note = Z00014|IGKV1-8*01
AIRMTQSPSSFSASTGDRVTITCRASQGISSYLAWYQQKPGKAPKLLIYAASTLQSGVPSRFSGSGSGTDFTLTISCLQSEDFATYYCQQYYSYP

SEQ ID NO: 91 - protein, 95 aa, synthetic construct
  source 1..95: note = Z00013|IGKV1-9*01
DIQLTQSPSFLSASVGDRVTITCRASQGISSYLAWYQQKPGKAPKLLIYAASTLQSGVPSRFSGSGSGTEFTLTISSLQPEDFATYYCQQLNSYP

SEQ ID NO: 92 - protein, 95 aa, synthetic construct
  source 1..95: note = X17262|IGKV1D-13*01
AIQLTQSPSSLSASVGDRVTITCRASQGISSALAWYQQKPGKAPKLLIYDASSLESGVPSRFSGSGSGTDFTLTISSLQPEDFATYYCQQFNNYP

SEQ ID NO: 93 - protein, 95 aa, synthetic construct
  source 1..95: note = K01323|IGKV1D-16*01
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 94 - protein, 87 aa, synthetic construct
  source 1..87: note = X63392|IGKV1D-17*01
SAMSASVGDRVTITCRARQGISNYLAWFQQKPGKVPKHLIYAASSLQSGVPSRFSGSGSGTEFTLTISSLQPEDFATYYCLQHNSYP

SEQ ID NO: 95 - protein, 95 aa, synthetic construct
  source 1..95: note = X72817|IGKV1D-43*01
AIRMTQSPFSLSASVGDRVTITCWASQGISSYLAWYQQKPAKAPKLFIYYASSLQSGVPSRFSGSGSGTDYTLTISSLQPEDFATYYCQQYYSTP

SEQ ID NO: 96 - protein, 95 aa, synthetic construct
  source 1..95: note = Z00008|IGKV1D-8*01
VIWMTQSPSLLSASTGDRVTISCRMSQGISSYLAWYQQKPGKAPELLIYAASTLQSGVPSRFSGSGSGTDFTLTISCLQSEDFATYYCQQYYSFP

SEQ ID NO: 97 - protein, 100 aa, synthetic construct
  source 1..100: note = X12684|IGKV2-24*01
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFP

SEQ ID NO: 98 - protein, 100 aa, synthetic construct
  source 1..100: note = X12691|IGKV2D-28*01, X63397|IGKV2-28*01
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 99 - protein, 100 aa, synthetic construct
  source 1..100: note = U41645|IGKV2-29*02
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLP

SEQ ID NO: 100 - protein, 100 aa, synthetic construct
  source 1..100: note = X63403|IGKV2-30*01
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWP

SEQ ID NO: 101 - protein, 101 aa, synthetic construct
  source 1..101: note = X59311|IGKV2D-40*01, X59314|IGKV2-40*01
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFP

SEQ ID NO: 102 - protein, 100 aa, synthetic construct
  source 1..100: note = AP001216|IGKV2D-26*01
EIVMTQTPLSLSITPGEQASISCRSSQSLLHSDGYTYLYWFLQKARPVSTLLIYEVSNRFSGVPDRFSGSGSGTDFTLKISRVEAEDFGVYYCMQDAQDP

SEQ ID NO: 103 - protein, 100 aa, synthetic construct
  source 1..100: note = M31952|IGKV2D-29*01
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLP

SEQ ID NO: 104 - protein, 100 aa, synthetic construct
  source 1..100: note = X63402|IGKV2D-30*01
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWP

SEQ ID NO: 105 - protein, 95 aa, synthetic construct
  source 1..95: note = X01668|IGKV3-11*01
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 106 - protein, 95 aa, synthetic construct
  source 1..95: note = M23090|IGKV3-15*01, X72815|IGKV3D-15*01
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 107 - protein, 96 aa, synthetic construct
  source 1..96: note = X12686|IGKV3-20*01
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 108 - protein, 95 aa, synthetic construct
  source 1..95: note = X17264|IGKV3D-11*01
EIVLTQSPATLSLSPGERATLSCRASQGVSSYLAWYQQKPGQAPRLLIYDASNRATGIPARFSGSGPGTDFTLTISSLEPEDFAVYYCQQRSNWH

SEQ ID NO: 109 - protein, 96 aa, synthetic construct
  source 1..96: note = X12687|IGKV3D-20*01
EIVLTQSPATLSLSPGERATLSCGASQSVSSSYLAWYQQKPGLAPRLLIYDASSRATGIPDRFSGSGSGTDFTLTISRLEPEDFAVYYCQQYGSSP

SEQ ID NO: 110 - protein, 96 aa, synthetic construct
  source 1..96: note = X72820|IGKV3D-7*01
EIVMTQSPATLSLSPGERATLSCRASQSVSSSYLSWYQQKPGQAPRLLIYGASTRATGIPARFSGSGSGTDFTLTISSLQPEDFAVYYCQQDYNLP

SEQ ID NO: 111 - protein, 101 aa, synthetic construct
  source 1..101: note = Z00023|IGKV4-1*01
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 112 - protein, 95 aa, synthetic construct
  source 1..95: note = X02485|IGKV5-2*01
ETTLTQSPAFMSATPGDKVNISCKASQDIDDDMNWYQQKPGEAAIFIIQEATTLVPGIPPRFSGSGYGTDFTLTINNIESEDAAYYFCLQHDNFP

SEQ ID NO: 113 - protein, 95 aa, synthetic construct
  source 1..95: note = X12683|IGKV6D-21*01
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 114 - protein, 12 aa, synthetic construct
  source 1..12: note = Z70260|IGKJ2*02
CTFGQGTKLEIK

SEQ ID NO: 115 - protein, 12 aa, synthetic construct
  source 1..12: note = J00242|IGKJ3*01
FTFGPGTKVDIK

SEQ ID NO: 116 - protein, 12 aa, synthetic construct
  source 1..12: note = J00242|IGKJ4*01
LTFGGGTKVEIK

SEQ ID NO: 117 - protein, 12 aa, synthetic construct
  source 1..12: note = J00242|IGKJ5*01
ITFGQGTRLEIK

SEQ ID NO: 118 - protein, 12 aa, synthetic construct
  source 1..12: note = J00242|IGKJ2*01
YTFGQGTKLEIK

SEQ ID NO: 119 - protein, 12 aa, synthetic construct
  source 1..12: note = J00242|IGKJ1*01
WTFGQGTKVEIK

SEQ ID NO: 120 - protein, 98 aa, synthetic construct
  source 1..98: note = Qualifier ValueAL122127|IGHG1*05, D3H44, J00228|IGHG1*01, JN582178|IGHG1*04, Pertuzumab, Z17370|IGHG1*02,
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 121 - protein, 98 aa, synthetic construct
  source 1..98: note = Y14737|IGHG1*03
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 122 - protein, 98 aa, synthetic construct
  source 1..98: note = AF449616|IGHG2*03, AF449618|IGHG2*05, AL928742|IGHG2*06, J00230|IGHG2*01
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 123 - protein, 98 aa, synthetic construct
  source 1..98: note = AJ250170|IGHG2*02
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTV

SEQ ID NO: 124 - protein, 98 aa, synthetic construct
  source 1..98: note = AF449617|IGHG2*04
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTV

SEQ ID NO: 125 - protein, 98 aa, synthetic construct
  source 1..98: note = AJ390236|IGHG3*05, AJ390237|IGHG3*06, AJ390238|IGHG3*07, AJ390241|IGHG3*08, AJ390242|IGHG3*09, AJ390244|IGHG3*13, AJ390247|IGHG3*11, AJ390252|IGHG3*12, AJ390254|IGHG3*14, AJ390260|IGHG3*15, AJ390262|IGHG3*16, AJ390279|IGHG3*19, AL122127|IGHG3*10, X03604|IGHG3*01, X16110|IGHG3*03, X99549|IGHG3*04
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 126 - protein, 98 aa, synthetic construct
  source 1..98: note = AJ390272|IGHG3*17
ASTKGPSVFPLAPCSRSTSGGTAALGCLVKDYFPEPVTVSWNSGALTSGVHTFPAVLQSSGLYSLSSVVTVPSSNFGTQTYTCNVNHKPSNTKVDKRV

SEQ ID NO: 127 - protein, 98 aa, synthetic construct
  source 1..98: note = AJ390276|IGHG3*18
ASTKGPSVFPLAPCSRSTSGGTAALGCLVKDYFPEPVTVSWNSGALTSGVHTFPAVLQYSGLYSLSSVVTVPSSSLGTQTYTCNVNHKPSNTKVDKRV

SEQ ID NO: 128 - protein, 98 aa, synthetic construct
  source 1..98: note = AL928742|IGHG4*04, K01316|IGHG4*01
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 129 - protein, 107 aa, synthetic construct [sequence omitted]

SEQ ID NO: 130 - protein, 107 aa, synthetic construct
  source 1..107: note = M11736|IGKC*02
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 131 - protein, 107 aa, synthetic construct
  source 1..107: note = M11737|IGKC*03
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 132 - protein, 107 aa, synthetic construct
  source 1..107: note = AF017732|IGKC*04
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 133 - protein, 107 aa, synthetic construct
  source 1..107: note = AF113887|IGKC*05
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 134 - protein, 118 aa, synthetic construct
  source 1..118: note = CAT-2200
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLIHGVTRNWGQGTLVTVSS

SEQ ID NO: 135 - skipped

SEQ ID NO: 136 - protein, 110 aa, synthetic construct
  source 1..110: note = CAT-2200
NFMLTQPHSVSESPGKTVTISCTRSSGSLANYYVQWYQQRPGSSPTIVIFANNQRPSGVPDRFSGSIDSSSNSASLTISGLKTEDEADYYCQTYDPYSVVFGGGTKLTVL

SEQ ID NO: 137 - protein, 98 aa, synthetic construct
  source 1..98: note = Z73673|IGLV6-57*01
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSN

SEQ ID NO: 138 - protein, 98 aa, synthetic construct
  source 1..98: note = Z73653|IGLV1-36*01
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNG

SEQ ID NO: 139 - protein, 99 aa, synthetic construct
  source 1..99: note = M94116|IGLV1-40*01
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 140 - protein, 98 aa, synthetic construct
  source 1..98: note = Z73654|IGLV1-44*01
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNG

SEQ ID NO: 141 - protein, 98 aa, synthetic construct
  source 1..98: note = Z73663|IGLV1-47*01
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSG

SEQ ID NO: 142 - protein, 98 aa, synthetic construct
  source 1..98: note = Z73661|IGLV1-51*01
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSA

SEQ ID NO: 143 - protein, 99 aa, synthetic construct
  source 1..99: note = Z73657|IGLV2-11*01
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTF

SEQ ID NO: 144 - protein, 99 aa, synthetic construct
  source 1..99: note = Z73664|IGLV2-14*01
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTL

SEQ ID NO: 145 - protein, 99 aa, synthetic construct
  source 1..99: note = Z73642|IGLV2-18*01
QSALTQPPSVSGSPGQSVTISCTGTSSDVGSYNRVSWYQQPPGTAPKLMIYEVSNRPSGVPDRFSGSKSGNTASLTISGLQAEDEADYYCSLYTSSSTF

SEQ ID NO: 146 - protein, 99 aa, synthetic construct
  source 1..99: note = X14616|IGLV2-23*01
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTL

SEQ ID NO: 147 - protein, 99 aa, synthetic construct
  source 1..99: note = X97462|IGLV2-8*01
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNF

SEQ ID NO: 148 - protein, 95 aa, synthetic construct
  source 1..95: note = X57826|IGLV3-1*01
SYELTQPPSVSVSPGQTASITCSGDKLGDKYACWYQQKPGQSPVLVIYQDSKRPSGIPERFSGSNSGNTATLTISGTQAMDEADYYCQAWDSSTA

SEQ ID NO: 149 - protein, 95 aa, synthetic construct
  source 1..95: note = X97473|IGLV3-9*01
SYELTQPLSVSVALGQTARITCGGNNIGSKNVHWYQQKPGQAPVLVIYRDSNRPSGIPERFSGSNSGNTATLTISRAQAGDEADYYCQVWDSSTA

SEQ ID NO: 150 - protein, 96 aa, synthetic construct
  source 1..96: note = X97464|IGLV3-10*01
SYELTQPPSVSVSPGQTARITCSGDALPKKYAYWYQQKSGQAPVLVIYEDSKRPSGIPERFSGSSSGTMATLTISGAQVEDEADYYCYSTDSSGNH

SEQ ID NO: 151 - protein, 96 aa, synthetic construct
  source 1..96: note = Z73658|IGLV3-12*01
SYELTQPHSVSVATAQMARITCGGNNIGSKAVHWYQQKPGQDPVLVIYSDSNRPSGIPERFSGSNPGNTTTLTISRIEAGDEADYYCQVWDSSSDH

SEQ ID NO: 152 - protein, 96 aa, synthetic construct
  source 1..96: note = X97471|IGLV3-16*01
SYELTQPPSVSVSLGQMARITCSGEALPKKYAYWYQQKPGQFPVLVIYKDSERPSGIPERFSGSSSGTIVTLTISGVQAEDEADYYCLSADSSGTY

SEQ ID NO: 153 - protein, 96 aa, synthetic construct
  source 1..96: note = X56178|IGLV3-19*01
SSELTQDPAVSVALGQTVRITCQGDSLRSYYASWYQQKPGQAPVLVIYGKNNRPSGIPDRFSGSSSGNTASLTITGAQAEDEADYYCNSRDSSGNH

SEQ ID NO: 154 - protein, 96 aa, synthetic construct
  source 1..96: note = X71966|IGLV3-21*01
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDH

SEQ ID NO: 155 - protein, 94 aa, synthetic construct
  source 1..94: note = Z73666|IGLV3-22*01
SYELTQLPSVSVSPGQTARITCSGDVLGENYADWYQQKPGQAPELVIYEDSERYPGIPERFSGSTSGNTTTLTISRVLTEDEADYYCLSGDEDN

SEQ ID NO: 156 - protein, 96 aa, synthetic construct
  source 1..96: note = X97474|IGLV3-25*01
SYELMQPPSVSVSPGQTARITCSGDALPKQYAYWYQQKPGQAPVLVIYKDSERPSGIPERFSGSSSGTTVTLTISGVQAEDEADYYCQSADSSGTY

SEQ ID NO: 157 - protein, 94 aa, synthetic construct
  source 1..94: note = D86994|IGLV3-27*01
SYELTQPSSVSVSPGQTARITCSGDVLAKKYARWFQQKPGQAPVLVIYKDSERPSGIPERFSGSSSGTTVTLTISGAQVEDEADYYCYSAADNN

SEQ ID NO: 158 - protein, 103 aa, synthetic construct
  source 1..103: note = X57828|IGLV4-3*01
LPVLTQPPSASALLGASIKLTCTLSSEHSTYTIEWYQQRPGRSPQYIMKVKSDGSHSKGDGIPDRFMGSSSGADRYLTFSNLQSDDEAEYHCGESHTIDGQVG

SEQ ID NO: 159 - protein, 99 aa, synthetic construct
  source 1..99: note = Z73667|IGLV4-60*01
QPVLTQSSSASASLGSSVKLTCTLSSGHSSYIIAWHQQQPGKAPRYLMKLEGSGSYNKGSGVPDRFSGSSSGADRYLTISNLQLEDEADYYCETWDSNT

SEQ ID NO: 160 - protein, 99 aa, synthetic construct
  source 1..99: note = Z73648|IGLV4-69*01
QLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGI

SEQ ID NO: 161 - protein, 104 aa, synthetic construct
  source 1..104: note = Z73672|IGLV5-37*01
QPVLTQPPSSSASPGESARLTCTLPSDINVGSYNIYWYQQKPGSPPRYLLYYYSDSDKGQGSGVPSRFSGSKDASANTGILLISGLQSEDEADYYCMIWPSNAS

SEQ ID NO: 162 - protein, 104 aa, synthetic construct
  source 1..104: note = Z73668|IGLV5-39*01
QPVLTQPTSLSASPGASARFTCTLRSGINVGTYRIYWYQQKPGSLPRYLLRYKSDSDKQQGSGVPSRFSGSKDASTNAGLLLISGLQSEDEADYYCAIWYSSTS

SEQ ID NO: 163 - protein, 104 aa, synthetic construct
  source 1..104: note = Z73670|IGLV5-45*01
QAVLTQPASLSASPGASASLTCTLRSGINVGTYRIYWYQQKPGSPPQYLLRYKSDSDKQQGSGVPSRFSGSKDASANAGILLISGLQSEDEADYYCMIWHSSAS

SEQ ID NO: 164 - protein, 105 aa, synthetic construct
  source 1..105: note = Z73669|IGLV5-52*01
QPVLTQPSSHSASSGASVRLTCMLSSGFSVGDFWIRWYQQKPGNPPRYLLYYHSDSNKGQGSGVPSRFSGSNDASANAGILRISGLQPEDEADYYCGTWHSNSKT

SEQ ID NO: 165 - protein, 98 aa, synthetic construct
  source 1..98: note = X14614|IGLV7-43*01
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQ

SEQ ID NO: 166 - protein, 98 aa, synthetic construct
  source 1..98: note = Z73674|IGLV7-46*01
QAVVTQEPSLTVSPGGTVTLTCGSSTGAVTSGHYPYWFQQKPGQAPRTLIYDTSNKHSWTPARFSGSLLGGKAALTLSGAQPEDEAEYYCLLSYSGAR

SEQ ID NO: 167 - protein, 98 aa, synthetic construct
  source 1..98: note = Z73650|IGLV8-61*01
QTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGI

SEQ ID NO: 168 - protein, 104 aa, synthetic construct
  source 1..104: note = Z73675|IGLV9-49*01
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFV

SEQ ID NO: 169 - protein, 98 aa, synthetic construct
  source 1..98: note = Z73676|IGLV10-54*01
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLSA

SEQ ID NO: 170 - protein, 12 aa, synthetic construct
  source 1..12: note = X04457|IGLJ1*01
YVFGTGTKVTVL

SEQ ID NO: 171 - protein, 12 aa, synthetic construct
  source 1..12: note = M15641|IGLJ2*01
VVFGGGTKLTVL

SEQ ID NO: 172 - protein, 12 aa, synthetic construct
  source 1..12: note = M18338|IGLJ6*01
NVFGSGTKVTVL

SEQ ID NO: 173 - protein, 12 aa, synthetic construct
  source 1..12: note = X51755|IGLJ7*01
AVFGGGTQLTVL

SEQ ID NO: 174 - protein, 98 aa, synthetic construct
  source 1..98: note = CAT-2200
ASTKGPSVFPLAPSSKSTSGGTAALGCLVKDYFPQPVTVSWNSGALTSGVHTFPAVLQSSGLYSLSSVVTVPSSSLGTQTYICNVNHKPSNTKVDKKV

SEQ ID NO: 175 - protein, 106 aa, synthetic construct
  source 1..106: note = CAT-2200, D87017|IGLC3*04, J00253|IGLC2*01, X06875|IGLC2*02,
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 176 - protein, 104 aa, synthetic construct
  source 1..104: note = J00252|IGLC1*01
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 177 - protein, 106 aa, synthetic construct
  source 1..106: note = X51755|IGLC1*02
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 178 - protein, 104 aa, synthetic construct
  source 1..104: note = J00254|IGLC3*01
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 179 - protein, 106 aa, synthetic construct
  source 1..106: note = K01326|IGLC3*02
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 180 - protein, 106 aa, synthetic construct
  source 1..106: note = X06876|IGLC3*03
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 181 - protein, 106 aa, synthetic construct
  source 1..106: note = J03011|IGLC6*01
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 182 - protein, 106 aa, synthetic construct
  source 1..106: note = M61771|IGLC7*02, X51755|IGLC7*01
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 183 - protein, 106 aa, synthetic construct
  source 1..106: note = KM455557|IGLC7*03
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

(19) United States
(12) Patent Application Publication
(10) Pub. No.: US 2026/0265415 A1
(43) Pub. Date: Sep. 10, 2026

(54) Antigen-Binding Polypeptide Constructs Comprising Kappa And Lambda Light Chains And Uses Thereof

(71) Applicant: ZYMEWORKS BC INC., Vancouver (CA)

(72) Inventors: Dunja UROSEV, Vancouver (CA); Stacey A.L. TOM-YEW, Coquitlam (CA); Leonard G. PRESTA, San Francisco, CA (US); Mario SANCHES, Vancouver (CA)

(21) Appl. No.: 19/381,963

(22) Filed: Nov. 6, 2025

Related U.S. Application Data
(62) Division of application No. 17/241,181, filed on Apr. 27, 2021, now Pat. No. 12,540,198.
Application No. 17/241,181 is a division of application No. 15/765,574, filed on Oct. 7, 2016, now Pat. No. 11,161,915.
(60) Provisional application No. 62/239,206, filed on Oct. 8, 2015.
(60) Provisional application No. 62/261,769, filed on Dec. 1, 2015.

(51) Int. Cl. C07K 16/46; C07K 16/24; C07K 16/32

(52) U.S. Cl. CPC C07K 16/46; C07K 16/244; C07K 16/32; C07K 16/468; C07K 2317/21; C07K 2317/24; C07K 2317/31; C07K 2317/522; C07K 2317/55; C07K 2317/56; C07K 2317/92; C07K 2317/94

(57) ABSTRACT

Provided herein are multispecific antigen-binding polypeptide constructs comprising at least two different heterodimers, each comprising a heavy chain and a light chain. At least one heterodimer comprises a Fab region comprising a lambda light chain and at least one heterodimer comprises a Fab region comprising a kappa light chain. One or more of the immunoglobulin heavy and light chains that form the antigen-binding polypeptide construct comprise amino acid modifications that promote correct pairing between the heavy and light chains to form the desired multispecific antigen-binding polypeptide construct. The amino acid modifications may be in the C_{H} and/or C_{L} domains, in the V_{H} and/or V_{L} domains, or a combination thereof.

### CROSS-REFERENCE

This application is a divisional of U.S. application Ser. No. 17/241,181, filed Apr. 27, 2021, which is a divisional of U.S. application Ser. No. 15/765,574, filed Apr. 3, 2018, which is a U.S. National Phase of International Patent Application No.: PCT/CA2016/051183, filed Oct. 7, 2016, which claims priority to U.S. Provisional Patent Application No. 62/261,769, filed Dec. 1, 2015 and U.S. Provisional Patent Application No. 62/239,206, filed Oct. 8, 2015, all of which are hereby claimed and their disclosures incorporated herein by reference in their entirety.

### SEQUENCE LISTING

The instant application contains a Sequence Listing which has been submitted electronically in .xml format and is hereby incorporated by reference in its entirety. Said .xml copy, created on Mar. 13, 2026, is named ZYME038 sequence listing_updated and is 218,624 bytes in size.

### BACKGROUND

Bispecific antibodies are capable of binding to two different epitopes, and are often prepared based on the immunoglobulin heavy and light chains of two different mono-specific parent antibodies. The ability to bind to two different epitopes or antigens makes bispecific antibodies an attractive tool for therapeutic applications where there is a benefit to targeting more than one antigen or epitope in the treatment of disease. However, efficiently producing bispecific antibodies in a format that is similar to naturally occurring antibodies can be difficult, since antibody heavy chains have evolved to bind antibody light chains in a relatively promiscuous manner. As a result of this promiscuous pairing, concomitant expression of the two different heavy chains and two different light chains of a bispecific antibody naturally leads to the scrambling of heavy chain-light chain pairings. This scrambling remains a major challenge for the generation of bispecific therapeutics, where homogeneous pairing is an essential requirement for good manufacturability and biological efficacy.

Some approaches have been described to prepare bispecific antibodies in a format similar to naturally occurring antibodies. However, these approaches have been developed and exemplified for cases where both of the parent antibodies used to prepare the bispecific antibody have light chains of the kappa gene family.

Although the majority of known therapeutic antibodies (and thus potential parent antibodies) have kappa light chains, there are some that have lambda light chains. Kappa and lambda light chains differ from each other in both structure and sequence.

A review of various approaches to produce bispecific antibodies in a format similar to naturally occurring antibodies from two parent antibodies can be found in Klein et al., (2012) mAbs 4:6, 1-11. International Patent Application No. PCT/EP2011/056388 (WO 2011/131746) describes an in vitro method for generating a heterodimeric protein in which asymmetrical mutations are introduced into the CH3 regions of two monospecific starting proteins in order to drive directional “Fab-arm” or “half-molecule” exchange between two monospecific IgG4- or IgG4-like antibodies upon incubation under reducing conditions.

US Patent Publication No. 2009/0182127 (Novo Nordisk, Inc.) describes the generation of bi-specific antibodies by modifying amino acid residues at the Fc interface and at the CH1: CL interface of light-heavy chain pairs that reduce the ability of the light chain of one pair to interact with the heavy chain of the other pair. International Patent Publication Nos. WO2014/081955 (Amgen), and WO2014/150973 (Eli Lilly) describe amino acid residues in the lambda light chain that may potentially be modified to drive desired pairing specificity. Neither of these publications describe complementary amino acid modifications that can be used to prepare a bispecific antibody from one parent antibody with a kappa light chain and another parent antibody with a lambda light chain. International Patent Publication No. WO2012/131555 (Glenmark), describes replacing the interface between the heavy chain and lambda light chain of an antibody with that of a TCR (T-cell receptor) domain interface.

### SUMMARY

The present disclosure provides multispecific antigen-binding polypeptides comprising an immunoglobulin lambda light chain and an immunoglobulin kappa light chain. In one aspect, the antigen-binding polypeptide is a construct comprising a first heterodimer and a second heterodimer. In one embodiment, the first heterodimer (H1L1) comprises a first immunoglobulin heavy chain polypeptide sequence (H1), and an immunoglobulin lambda light chain polypeptide sequence (L1) that form a first Fab region that specifically binds to a first antigen; and the second heterodimer (H2L2) comprises a second immunoglobulin heavy chain polypeptide sequence (H2), and an immunoglobulin kappa light chain polypeptide sequence (L2) that form a second Fab region that specifically binds to a second antigen. In some embodiments, H1 is distinct from H2. In some embodiments, H1 and H2 comprise a heavy chain variable domain (VH domain) and a heavy chain constant domain 1 (CH1 domain). In one embodiment, L1 comprises a lambda light chain variable (VL-lambda) domain and a lambda light chain constant (CL-lambda) domain. In one embodiment, L2 comprises a kappa light chain variable (VL-kappa) domain and a kappa light chain constant (CL-kappa) domain. In some embodiments, one or more of H1, H2, L1, and L2 comprise amino acid modifications as compared to corresponding wild type H1, H2, L1, and L2 polypeptide sequences, wherein the amino acid modifications promote preferential pairing of H1 with L1 as compared to L2, and/or promote preferential pairing of H2 with L2 as compared to L1. In some embodiments, the amino acid modifications do not introduce a new cysteine residue. In one embodiment, the amino acid modifications do not remove a naturally occurring cysteine residue.

In some embodiments, the construct comprises amino acid modifications that promote preferential pairing of H1 with L1 as compared to L2, and/or that promote preferential pairing of H2 with L2 as compared to L1, when H1, H2, L1 and L2 are co-expressed in a cell or a mammalian cell, or when H1, H2, L1 and L2 are co-expressed in a cell-free expression system, or when H1 and L1 are produced in a (first) cell and H2 and L2 are produced in a second (e.g., different) cell and the products of the two cells are mixed via a redox production method, or when H1 and L1 are produced in a first cell-free expression system and H2 and L2 are produced in a second (e.g., different) cell-free expression system and the products of the two cell-free expression systems are mixed.

In some embodiments of the construct, each heterodimer comprises a single Fab.

In some embodiments of the antigen-binding polypeptide constructs described herein:
a. H2 comprises amino acid substitution at position 143; L2 comprises an amino acid substitution at position 124; and
   i. H1 comprises amino acid substitution at position 186 or 179, and L1 comprises amino acid substitution at position 180;
   ii. H1 comprises amino acid substitution at position 186; and L1 comprises amino acid substitutions at position 133;
   iii. H1 comprises amino acid substitution at position 143; and L1 comprises amino acid substitutions at position 133; or
   iv. H1 comprises amino acid substitution at position 188; and L1 comprises amino acid substitutions at position 178;
b. H1 comprises amino acid substitution at position 143; L1 comprises amino acid substitution at position 131; and
   i. H2 comprises amino acid substitution at positions 186, or 124 and 186, and L2 comprises amino acid substitution at positions 133, or 133 and 160, or 124 and 133, or 176 and 180; or
   ii. H2 comprises amino acid substitution at position 188; and L2 comprises amino acid substitutions at position 131;
   iii. H2 comprises amino acid substitution at position 143; and L2 comprises amino acid substitutions at positions 124 and 133, or 124 and 133 and 180;
c. H1 comprises amino acid substitution at position 143; L1 comprises amino acid substitution at position 131; and
   i. H2 comprises amino acid substitution at positions 124 and 186, or 124 and 179, or 188, and L2 comprises amino acid substitution at positions 176 and 178, or 176 and 180, or 131; or
   ii. H2 comprises amino acid substitution at positions 143 and 188, or 143, or 124 and 143; and L2 comprises amino acid substitutions at positions 124 and 176 and 178, or 124 and 178, or 124 and 180, or 124 and 176 and 180, or 124, or 124 and 176;
d. H1 comprises amino acid substitution at position 179, 186, 143 and/or 188; L1 comprises amino acid substitution at position 180, 133 and/or 176 and 178; H2 comprises amino acid substitution at position 143, and L2 comprises amino acid substitution at position 131 and/or 124;
e. H1 comprises amino acid substitution at position 39 or comprises no amino acid substitutions that promote preferential pairing; L1 comprises amino acid substitution at position 38 or comprises no amino acid substitutions that promote preferential pairing; H2 comprises amino acid substitution at position 39, and L2 comprises amino acid substitution at position 38;
f. H1 comprises amino acid substitution at position 143; L1 comprises amino acid substitution at position 131; and
   i. H2 comprises amino acid substitution at positions 188 or 124 and 186, and L2 comprises amino acid substitution at positions 176 and 178, or 176 and 180, or 131; or
   ii. H2 comprises amino acid substitution at position 143 or 186; and L2 comprises amino acid substitutions at positions 124 and 133, or 124 and 133 and 180;
g. H1 comprises amino acid substitution at position 188; L1 comprises amino acid substitution at positions 176 and 178, or 178; and
   i. H2 comprises amino acid substitution at positions 177 and 188, and L2 comprises amino acid substitution at positions 176 and 178; or
   ii. H2 comprises amino acid substitution at position 186 or 124 or 124 and 179; and L2 comprises amino acid substitutions at positions 176, or 131 and 176;
h. H1 comprises amino acid substitution at position 186; L1 comprises amino acid substitution at position 133; and
   i. H2 comprises amino acid substitution at position 188, and L2 comprises amino acid substitution at position 131; or
   ii. H2 comprises amino acid substitution at positions 177 and 188; and L2 comprises amino acid substitutions at positions 176 and 178; or
   iii. H1 comprises amino acid substitution at positions 124 and 190; L1 comprises amino acid substitution at position 135; H2 comprises amino acid substitution at positions 124 or 188, and L2 comprises amino acid substitution at positions 176, or 176 and 178;
i. H1 comprises amino acid substitution at positions 177 and 188; L1 comprises amino acid substitution at position 176 and 178; and
   a. H2 comprises amino acid substitution at position 188, and L2 comprises amino acid substitution at positions 176 and 178, or 131;
   b. H2 comprises amino acid substitution at position 186; and L2 comprises amino acid substitutions at positions 133, or 124 and 160 and 180;
   c. H2 comprises amino acid substitution at position 124, or 124 and 179, or 124 and 186; and L2 comprises amino acid substitutions at positions 176, or 176 and 178, or 176 and 180; or
   d. H2 comprises amino acid substitution at position 143; and L2 comprises amino acid substitutions at positions 133, or 124 and 133;
j. H1 comprises amino acid substitution at position 188; L1 comprises amino acid substitution at position 178; H2 comprises amino acid substitution at position 124 or 188, and L2 comprises amino acid substitution at positions 176 and 178, or 176 and 180, or 176;
k. or H1 comprises amino acid substitution at positions 145 and 188; L1 comprises amino acid substitution at position 178; and H2 comprises amino acid substitution at position 124 and/or 188, and L2 comprises amino acid substitution at one or more of positions 124, 133, and 178;
l. H1 comprises amino acid substitution at position 174, 179 or 186; L1 comprises amino acid substitution at position 176 or 180; H2 comprises amino acid substitution at position 143 or 190, and L2 comprises amino acid substitution at position 131, 135 or 124; or
m. H1 comprises amino acid substitution at position 174; L1 comprises amino acid substitution at position 176; H2 comprises amino acid substitution at position 190; and L2 comprises no amino acid substitutions that promote preferential pairing or comprises amino acid substitution at position 135;
n. H1 comprises amino acid substitution at positions 143 and 190; L1 comprises amino acid substitution at position 133; H2 comprises amino acid substitution at position 124, and L2 comprises amino acid substitution at positions 131 and 135;
o. H1 comprises amino acid substitution at position 143 and/or 186; L1 comprises amino acid substitution at position 133; H2 comprises amino acid substitution at position 124, and L2 comprises amino acid substitution at position 131;
p. H1 comprises amino acid substitution at positions 143 and 179; L1 comprises amino acid substitution at positions 124 and 178; H2 comprises amino acid substitution at position 186, and L2 comprises amino acid substitution at positions 178 and 180, or 160 and 180;
q H1 comprises amino acid substitution at position 143; L1 comprises amino acid substitution at position 124; H2 comprises amino acid substitution at position 179 or 186, and L2 comprises amino acid substitution at position 124 and 160 and 180;
r. H1 comprises amino acid substitution at position 186; L1 comprises amino acid substitution at positions 180 or 178 and 180; H2 comprises amino acid substitution at position 143 and/or 179, and L2 comprises amino acid substitution at positions 124 and 178, or 131;
s. H1 comprises amino acid substitution at position 179; L1 comprises amino acid substitution at position 180; H2 comprises amino acid substitution at position 143, and L2 comprises amino acid substitution at position 124;
t. H1 comprises amino acid substitution at position 143 or 186; L1 comprises amino acid substitution at position 180 or comprises no amino acid substitutions that promote preferential pairing; H2 comprises amino acid substitution at positions 143 and 145, and L2 comprises amino acid substitution at position 124;
u. H1 comprises no amino acid substitutions that promote preferential pairing; L1 comprises amino acid substitution at position 135; H2 comprises amino acid substitution at position 139, and L2 comprises amino acid substitution at position 116;
v. H1 comprises no amino acid substitutions that promote preferential pairing or comprises amino acid substitution at position 45; L1 comprises no amino acid substitutions that promote preferential pairing; H2 comprises amino acid substitution at position 45, and L2 comprises amino acid substitution at position 44;
w. H1 comprises amino acid substitution at position 139; L1 comprises amino acid substitution at position 116; H2 comprises no amino acid substitutions that promote preferential pairing, and L2 comprises amino acid substitution at position 135; or
x. H1 comprises amino acid substitution at position 124; L1 comprises amino acid substitution at position 176; H2 comprises amino acid substitution at position 124, and L2 comprises amino acid substitution at position 176.

In some embodiments, the affinity of the first Fab region for the first antigen is within about 1, 2, 3, 4, 5, 6, 7, 8, 9, 10, 20, 25, 30, 35, 40, 45, 50 or 100-fold of the affinity of a Fab region formed by the corresponding wild type H1 and L1 polypeptide sequences for the first antigen, and/or the affinity of the second Fab region for the second antigen is within about 1, 2, 3, 4, 5, 6, 7, 8, 9, 10, 20, 25, 30, 35, 40, 45, 50 or 100-fold of the affinity of a Fab region formed by the corresponding wild type H2 and L2 polypeptide sequences for the second antigen.

In some embodiments, the melting temperature (Tm) of the first Fab region is within about 0, 1, 2, 3, 4, 5, 6, 7, 8, 9, 10, or 20° C. of the Tm of a Fab region formed by the corresponding wild type H1 and L1 polypeptide sequences for the first antigen, and/or the melting temperature (Tm) of the second Fab region is within about 0, 1, 2, 3, 4, 5, 6, 7, 8, 9, 10, or 20° C. of the Tm of a Fab region formed by the corresponding wild type H2 and L2 polypeptide sequences for the second antigen.

In some embodiments of the antigen-binding polypeptide construct:
a. H1 and L1 are wild type polypeptide sequences and H2 and L2 each comprise at least one amino acid modification;
b. one or more of H1, L1, and H2 comprise at least one amino acid modification, and L2 is a wild type polypeptide sequence;
c. one or more of H1, L1, and L2 comprise at least one amino acid modification, and H2 is a wild type polypeptide sequence;
d. one or more of H1, H2, and L2 comprise at least one amino acid modification and L1 is a wild type polypeptide sequence;
e. one or more of L1, H2, and L2 comprise at least one amino acid modification and H1 is a wild type polypeptide sequence; or
f. H1, L1, H2, and L2 each comprise at least one amino acid modification.

In some embodiments, the amino acid modifications are in:
a. the CH1 domains of H1 and H2, the CL-lambda domain of L1 and the CL-kappa domain of L2; or
b. the CH1 and VH domains of H1 and H2, the CL-lambda domain and VL-lambda domain of L1, and the CL-kappa domain and VL-kappa domain of L2.

In some embodiments, the amino acid modifications are in:
a. at least two of the CH1 domain of H1, the CH1 domain of H2, the CL-lambda domain of L1 and the CL-kappa domain of L2;
b. at least two of the CH1 and VH domains of H1 and H2, the CL-lambda domain and VL-lambda domain of L1, and the CL-kappa domain and VL-kappa domain of L2, or
c. at least two of the VH domain of H1, the VH domain of H2, the VL-lambda domain of L1, and the VL-kappa domain of L2.

In some embodiments, H1, L1, H2, and/or L2 comprises at least 1, 2, 3, 4, 5, 6, 7, or 8, amino acid mutations in the Fab region. In some embodiments, at least one of H1, H2, L1 and L2 comprises at least 2, 3, 4, 5, 6, 7, 8, 9, or 10 amino acid modifications of at least one constant domain and/or at least one variable domain.

In one embodiment, one or more of H1, H2, L1 and L2 comprise amino acid modifications that promote preferential pairing of H1 with L1 as compared to L2 to form H1L1, or H2 with L2 as compared to L1 to form H2L2, such that the relative pairing of at least one of H1L1 or H2L2 is at least about 10% greater relative to wild-type, and the relative pairing of the other is within about 10% of wild-type or at least about 10% greater relative to wild-type.

In some embodiments, the amino acid modifications promote preferential pairing of H1 with L1 as compared to L2 to form H1L1, or H2 with L2 as compared to L1 to form H2L2, where the ratio of H1L1: H1L2 is at least 40:60 and the ratio of H2L2:H2L1 is at least 60:40; or the amino acid modifications promote preferential pairing of H1 with L1 as compared to L2 to form H1L1, or H2 with L2 as compared to L1 to form H2L2, where the ratio of H2L2:H2L1 is at least 40:60 and the ratio of H1L1: H1L2 is at least 60:40.

In some embodiments, the thermal stability of the first Fab region is within about 0, 1, 2, or 3° C. of the Tm of a Fab region formed by the corresponding wild type H1 and L1 polypeptide sequences. In some embodiments, the thermal stability of the second Fab region is within about 0, 1, 2, or 3° C. of the Tm of the Fab region formed by the corresponding wild type H2 and L2 polypeptide sequences.

In some embodiments, the amino acid modifications are selected from the group consisting of the unique identifier Mab design sets shown in Table 4A or 4B. In some embodiments, the amino acid modifications are selected from the group consisting of the unique identifier Mab design sets shown in one or more of Tables 10-A1 to 10-A12. In some embodiments, the amino acid modifications are selected from the group consisting of the unique identifier Mab design sets shown in any one of Tables 10-B1 to 10-B10.

In some embodiments, the construct further comprises a dimeric Fc having two Fc polypeptides each comprising a CH3 domain sequence and coupled with or without linkers to one of the first Fab region and second Fab region. In some embodiments, the Fc is a human Fc, a human IgG1 Fc, a human IgA Fc, a human IgG Fc, a human IgD Fc, a human IgE Fc, a human IgM Fc, a human IgG2 Fc, a human IgG3 Fc, or a human IgG4 Fc. In one embodiment, the Fc comprises one or more modifications as compared to wild type, in at least one of the CH3 domain sequences that promote the formation of a heterodimeric Fc.

In some embodiments, the Fc comprises:
i) a heterodimeric IgG1 Fc having the modifications L351Y_F405A_Y407V in the first Fc polypeptide, and the modifications T366L_K392M_T394W in the second Fc polypeptide;
ii) a heterodimeric IgG1 Fc having the modifications L351Y_F405A_Y407V in the first Fc polypeptide, and the modifications T366L_K392L_T394W in the second Fc polypeptide;
iii) a heterodimeric IgG1 Fc having the modifications T350V_L351Y_F405A_Y407V in the first Fc polypeptide, and the modifications T350V_T366L_K392L_T394W in the second Fc polypeptide;
iv) a heterodimeric IgG1 Fc having the modifications T350V_L351Y_F405A_Y407V in the first Fc polypeptide, and the modifications T350V_T366L_K392M_T394W in the second Fc polypeptide; or
v) a heterodimeric IgG1 Fc having the modifications T350V_L351Y_S400E_F405A_Y407V in the first Fc polypeptide, and the modifications T350V_T366L_N390R_K392M_T394W in the second Fc polypeptide.

In some embodiments, the Fc further comprises at least one CH2 domain sequence. In one embodiment, the Fc comprises one or more modifications to promote selective binding of Fc-gamma receptors, to reduce or eliminate binding to Fc-gamma receptors, or to promote binding to FcRn.

In some embodiments, when H1, L1, H2 and L2 are co-expressed, the change in the amount of total correct pairing as measured by the sum of HILL and H2L2 pairing is greater than about 5%, 10%, 15%, 20%, 25%, 30%, 35%, 40%, or 45% compared to the pairing of corresponding H1, L1, H2 and L2 polypeptide chains without amino acid substitutions in the Fab region that promote preferential pairing; or the change in the amount of total correct pairing, as measured by the amount of bispecific antibody produced as a percentage of species other than half-antibodies produced, is greater than about 5%, 10%, 20%, 30%, 40%, 50%, 60%, 70%, or 80%, compared to the pairing of corresponding H1, L1, H2 and L2 polypeptide chains without amino acid substitutions in the Fab region that promote preferential pairing, or the change in the amount of total correct pairing, as measured by the amount of bispecific antibody produced as a percentage of all species produced, is greater than about 5%, 10%, 20%, 30%, 40%, 50%, 60%, 70%, or 80%, compared to the pairing of corresponding H1, L1, H2 and L2 polypeptide chains without amino acid substitutions in the Fab region that promote preferential pairing.

In some embodiments, the linkers comprise one or more polypeptide linkers, one or more antibody hinge regions, or one or more IgG1 hinge regions. In one embodiment, the one or more polypeptide linkers comprise one or more modifications as compared to a wild type polypeptide linker.

In some embodiments, the amino acid modifications comprise amino acid substitutions.

In some embodiments, the sequences of one or more of H1, H2, L1, and L2 are derived from human sequences or humanized sequences.

In some embodiments, the constructs described herein are conjugated to a therapeutic agent or drug.

In another aspect, the disclosure provides an isolated recombinant polynucleotide or set of isolated recombinant polynucleotides that encode(s) the constructs described herein. In some embodiments, provided are a vector or a set of vectors comprising one or more of the polynucleotides or sets of polynucleotides described herein. In some embodiments, the vector or at least one vector of the set of vectors is multi-cistronic.

In another aspect, the disclosure provides an isolated cell comprising the polynucleotide or set of polynucleotides, or the vector or set of vectors described herein. In some embodiments, the cell is a yeast cell, a bacterial cell, an insect cell, or a mammalian cell. In some embodiments, the isolated cell is stably transfected or transiently transfected with the vector or set of vectors described herein.

In another aspect, a pharmaceutical composition comprising the antigen-binding polypeptide constructs described herein is provided. In some embodiments, the pharmaceutical composition comprises a pharmaceutically acceptable carrier. In some embodiments, the pharmaceutical composition further comprises one or more substances selected from the group consisting of a buffer, an antioxidant, a low molecular weight molecule, a drug, a protein, an amino acid, a carbohydrate, a lipid, a chelating agent, a stabilizer, and an excipient.

In another aspect, a method of preparing the constructs described herein is described. In some embodiments, the method comprises the steps of:
(a) obtaining a host cell comprising a polynucleotide or set of polynucleotides encoding the antigen-binding polypeptide construct;
(b) culturing the host cell in a host cell culture under conditions that allow expression of the antigen-binding polypeptide construct, and
(c) collecting the antigen-binding polypeptide construct from the host cell culture.

In some embodiments, the host cell is transiently transfected or stably transfected with the polynucleotide or set polynucleotides described herein.

In another aspect, a computer-readable storage medium is provided. In some embodiments, the computer-readable storage medium stores a dataset comprising data representing complementary amino acid modifications in a first heterodimer comprising a first immunoglobulin heavy chain polypeptide sequence (H1) and a first immunoglobulin lambda light chain polypeptide sequence (L1); and/or a second heterodimer comprising a second immunoglobulin heavy chain polypeptide sequence (H2) and a second immunoglobulin kappa light chain polypeptide sequence (L2). In some embodiments, the H1 and H2 polypeptide sequences stored in the dataset comprise at least a heavy chain variable domain (VH domain) and a heavy chain constant domain (CH1 domain) and are distinct from each other. In some embodiments, the L1 and L2 polypeptide sequences stored in the dataset comprise at least a light chain variable domain (VL domain) and a light chain constant domain (CL domain). In some embodiments, the complementary amino acid modifications stored in the dataset promote preferential pairing of H1 with L1 as compared to L2, and of H2 with L2 as compared to L1. In some embodiments, the dataset comprises data representing those modifications listed in Table 4A or Table 4B or a subset of those modifications. In some embodiments, the dataset comprises data representing those modifications listed in one or more of Tables 10-A1 to 10-A12 or Tables 10-B1 to 10-B10, or a subset of those modifications

In another aspect, a method of producing a bispecific antigen-binding polypeptide construct is described. In some embodiments, the bispecific antigen-binding polypeptide construct produced by the method comprises:
a. a first heterodimer comprising a first immunoglobulin heavy chain polypeptide sequence (H1) and a first immunoglobulin lambda light chain polypeptide sequence (L1); and
b. a second heterodimer comprising a second immunoglobulin heavy chain polypeptide sequence (H2) and a second immunoglobulin kappa light chain polypeptide sequence (L2).

In some embodiments, the H1 and H2 polypeptide sequences produced by the method comprise at least a heavy chain variable domain (VH domain) and a heavy chain constant domain (CH1 domain) and are distinct from each other. In some embodiments, the L1 and L2 polypeptide sequences produced by the method comprise a light chain variable domain (VL domain) and a light chain constant domain (CL domain). In some embodiments, one or more of H1, L1, H2, and L2 polypeptide sequences produced by the method comprise amino acid modifications that promote preferential pairing of H1 with L1 as compared to L2 and of H2 with L2 as compared to L1.

In some embodiments, the method of producing the bispecific antigen-binding polypeptide construct comprises:
a. introducing one or more complementary amino acid modifications from the dataset described herein into H1, L1, H2 and/or L2; and
b. co-expressing H1, L1, H2 and L2 in a host cell to produce an expression product comprising the bi-specific antigen-binding polypeptide construct.

In some embodiments, the method further comprises determining the amount of the bispecific antigen-binding polypeptide construct in the expression product relative to other polypeptide products to select a preferred subset of complementary amino acid modifications that provide an increased amount of the bispecific antigen-binding polypeptide construct as compared to the amount of bispecific antigen-binding polypeptide construct in an expression product resulting from the co-expression of wild type H1, L1, H2 and L2. In some embodiments, the bispecific antigen-binding polypeptide construct is produced with a purity of greater than 70% compared to the other polypeptide products. In some embodiments, the construct produced by the method comprises an Fc comprising at least two CH3 domain sequences, and the Fc is coupled, with or without one or more linkers, to the first heterodimer and the second heterodimer. In some embodiments, the Fc is a heterodimeric Fc comprising one or more amino acid modifications that promote formation of a heterodimeric Fc over a homodimeric Fc.

In some embodiments of the method for producing the bispecific antigen-binding polypeptide construct, when H1, L1, H2 and L2 are co-expressed, the change in the amount of total correct pairing as measured by the sum of % H1L1 and % H2L2 produced is greater than about 5%, 10%, 15%, 20%, 25%, 30%, 35%, 40%, or 45% compared to the pairing of corresponding H1, L1, H2 and L2 polypeptide chains without amino acid substitutions in the Fab region that promote preferential pairing; or the change in the amount of total correct pairing as measured by the amount of bispecific antibody produced as a percentage of species other than half-antibodies produced is greater than about 5%, 10%, 20%, 30%, 40%, 50%, 60%, 70%, or 80%, compared to the pairing of corresponding H1, L1, H2 and L2 polypeptide chains without amino acid substitutions in the Fab region that promote preferential pairing; or the change in the amount of total correct pairing as measured by the amount of bispecific antibody produced as a percentage of all species produced is greater than about 5%, 10%, 20%, 30%, 40%, 50%, 60%, 70%, or 80%, compared to the pairing of corresponding H1, L1, H2 and L2 polypeptide chains without amino acid substitutions in the Fab region that promote preferential pairing.

### BRIEF DESCRIPTION OF THE FIGURES

FIG. 1A-1H depict D3H44, Pertuzumab, and CAT-2200 heavy chain and light chain amino acid sequences aligned against human germline sequences for Variable and Constant domains. Translated protein sequences for each Domain, Germline, and Allele were obtained by directly querying the IMGT/GENE-DB (imgt.org/genedb/query). IMGT/DomainGapAlign (imgt.org/3Dstructure-DB/cgi/DomainGapAlign.cgi) was used to determine the closest gene/allele. Identification of consensus sequences was performed by BoxShade (ch.embnet.org/software/BOX_form.html) with a 0.8 cut-off. Amino acid residues shaded in black represent amino acid sequence identity, while those shaded in gray represent amino acid sequence similarity. The assignment of amino acids to each domain in FIG. 1A-1H was made according to IMGT definitions as described in Lefranc M.-P. et al. “IMGT unique numbering for immunoglobulin and T cell receptor constant domains and Ig superfamily C-like domains” Dev. Comp. Immunol., 2005, 29, 185-203, and Lefranc, M.-P., Pommié, C., Ruiz, M., Giudicelli, V., Foulquier, E., Truong, L., Thouvenin-Contet, V. and Lefranc, G. “IMGT unique numbering for immunoglobulin and T cell receptor variable domains and Ig superfamily V-like domains” Dev. Comp. Immunol., 27, 55-77 (2003). FIG. 1A depicts Pertuzumab and D3H44 variable heavy (VH) domains aligned against human IGHV and IGHJ germline subgroups (one representative sequence is displayed for each gene and allele). The closest gene and allele sequences to Pertuzumab IGHV and IGHJ are X92218|IGHV3-66*01 and J00256|IGHJ4*01, respectively. The closest gene and allele sequences to D3H44 IGHV and IGHJ are X92218|IGHV3-66*01 and J00256|IGHJ4*01, respectively. FIG. 1B depicts Pertuzumab and D3H44 variable light (VL) domains aligned against human kappa IGKV and IGKJ germline subgroups (one representative sequence is displayed from each gene and allele). The closest gene and allele sequences to Pertuzumab IGKV and IGKJ are Y14865|IGKV1-NL1*01 and J00242|IGKJ2*01, respectively. The closest gene and allele sequences to D3H44 IGKV and IGKJ are X59315|IGKV1-39*01 and J00242|IGKJ1*01, respectively. FIG. 1C depicts Pertuzumab and D3H44 constant heavy 1 (CH1) domains aligned against human CH1 IGHG germline subgroups. The closest gene and allele sequence to Pertuzumab and D3H44 IGHG is J00228|IGHG1*01. FIG. 1D depicts Pertuzumab and D3H44 constant light (CL) domains aligned against human kappa IGKC germline subgroups. The closest gene and allele sequence to Pertuzumab and D3H44 IGKC is J00241|IGKC*01. FIG. 1E depicts CAT-2200 VH domain aligned against human IGHV and IGHJ germline subgroups (one representative sequence is displayed for each gene and allele). The closest gene and allele sequences to CAT-2200 IGHV and IGHJ are M99660|IGHV3-23*01 and J00256|IGHJ4*01, respectively. FIG. 1F depicts CAT-2200 VL domain aligned against human lambda IGLV and IGLJ germline subgroups (one representative sequence is displayed from each gene and allele). The closest gene and allele sequences to CAT-2200 IGLV and IGLJ are Z73673|IGLV6-57*01 and M15641|IGLJ2*01, respectively. FIG. 1G depicts CAT-2200 CH1 domain aligned against human CH1 IGHG germline subgroups. The closest gene and allele sequence to CAT-2200 IGHG is J00228|IGHG1*01. FIG. 1H depicts CAT-2200 CL domain aligned against human lambda IGLC germline subgroups. The closest gene and allele sequence to CAT-2200 IGLC is J00253|IGLC2*01.

FIG. 2 depicts a flowchart for identifying interface residues and for computational modeling of designs with preferential heavy-light chain pairing.

FIGS. 3A and 3B depict a 3D structural alignment between the constant domains of D3H44 (PDB ID 1JPT) and CAT-2200 (PDB-ID 2VXS). FIG. 3A exemplifies the typical conformational differences seen between a kappa and lambda light chain when aligned on their respective heavy chain. FIG. 3B presents a view of the light chain interface (with heavy chain removed) of the model presented in FIG. 3A, to further exemplify the conformational differences. The dotted arrows point to the conformational rearrangement of the secondary structure elements at the interface between heavy and light chains.

FIG. 4 illustrates a high level schematic overview of the engineering requirements for forming a bispecific antibody, and the assay requirements needed to quantify heavy chain light chain (H-L) pairs. The design goal of engineering a bispecific antibody with high purity (i.e., little or no mispaired H-L associations) can be achieved by rationally engineering (via the introduction of specific amino acid mutations) the preferential pairing of two unique heavy chains for their unique cognate light chains. This process is shown schematically; here H1 has been engineered to preferentially pair with L1 (indicated by a checkmark) and not L2 (indicated by an “X”). Likewise, H2 has been engineered to preferentially pair with L2 and not L1. The arrows on the HILL and H2L2 heterodimers represent facilitated pairing between these H-L pairs, while the arrows on the H1L2 and H2L1 heterodimers represent disruption of pairing between the latter H-L pairs. The experimental screening of designs to promote preferential pairing requires an assay capable of simultaneously quantifying H1L1: H1L2 and H2L2:H2L1. These assay requirements can be simplified by assuming that each bispecific Fab arm can be independently engineered. In this case, the assay would only need to quantify H1L1: H1L2 or H2L2:H2L1, and not both simultaneously.

FIG. 5 provides a schematic depicting how heavy chains and light chains can be tagged and how preferential pairing is determined. In this schematic, the circular boundary represents a cell in which 3 constructs (one heavy chain and two unique light chains) are transfected. The expression products are secreted from the cell and the supernatant (SPNT) is passed over a detection device, in this case an SPR chip. Based on detection of the two different tags fused to the two light chains competing for heavy chain pairing, a quantitative estimate of the preferential pairing of the heavy chain to the two light chains can be obtained.

FIG. 6 depicts the performance filtering criteria based on the two LCCA results for each design. These performance filtering criteria were used to identify the K-L design library and the K-K-derived K-L design library. To be included, a design should contain a positive LCCA result above the neutral zone (neutral zone defined as the region between 40:60 and 60:40 paired: mispaired ratios) while the other LCCA has to be above the lower limit of the neutral zone (the 40:60 paired: mispaired ratio). Scenarios A and B represent designs that pass the filtering criteria. Note that Scenario B is included because H2L2:H2L1 LCCA is above the neutral zone and H1L1: H1L2 LCCA inside the neutral zone (not below it). Scenario C represents a design that is filtered out, where both LCCA results are positive, but neither are above the neutral zone. Scenarios D and E represent designs that are filtered out because at least one of the LCCA results is below the neutral zone, even if the other LCCA is above the neutral zone (see Scenario D). Designs where the designation of H1L1 and H2L2 are reversed are also included. This description of this figure. assumes that the wild-type pairing ratio is 50:50.

FIG. 7 depicts the performance of the selected K-L designs as well as K-K-derived K-L designs (based on LCCA data for Mab design set in Tables 4A and 4B) as defined by the design strength=ΔH1:L1:L2_scalar+ΔH2:L2:L1 scalar. This metric is an indicator of the overall pairing success at the design level.

FIG. 8 depicts the potential heavy chain associated products that can be expected when two different light chains are co-expressed with two different heavy chains in a cell.

FIG. 9 depicts a general method of preparing a bispecific antigen-binding polypeptide construct using the Mab design set library provided herein.

FIGS. 10A and 10B depict min-max box plots summarizing the performance of all K-L designs tested in the SMCA in three bispecific systems, by design cluster. FIG. 10A shows performance as measured by the total bispecific calculation (ΔBispecific %). FIG. 10B shows performance as measured by the total pairing calculation (ΔPairing %).

FIG. 11 depicts a min-max box plot graph summarizing the performance of K-L designs and K-K-derived K-L designs per bispecific system, by transferability group; “kl 3/3” indicates K-L designs transferable in 3/3 bispecific systems, “kl 3/3+2/3” indicates K-L designs transferable in at least 2 bispecific systems, and “kl all” indicates all K-L designs tested. Likewise, “kk 3/3” indicates K-K-derived K-L designs transferable in 3/3 bispecific systems, “kk 3/3+2/3” indicates K-K-derived K-L designs transferable in at least 2 bispecific systems, and “kk all” indicates all K-K-derived K-L designs tested; results are presented based on the total bispecific calculation (ΔBispecific %).

FIGS. 12A-12C depict DSC sensorgrams of the bispecific antibodies produced using Mab design set 3972 (SMCA design ID) (in each of three bispecific systems), and the wild-type parent antibodies for each system. FIG. 12A depicts wild-type CAT-2200 mAb (dark grey), wild-type Pertuzumab mAb (medium grey), and Design 3972 CAT-2200/Pertuzumab SMCA (light grey); FIG. 12B depicts wild-type CAT-2200 mAb (dark grey), wild-type SGN-CD19a mAb (medium grey), and Design 3972 CAT-2200/SGN-CD19a SMCA (light grey); FIG. 12C depicts wild-type SGN-CD19a mAb (dark grey), wild-type CR8071 mAb (medium grey), and Design 3972 CR8071/SGN-CD19a SMCA (light grey).

FIG. 13 depicts a min-max box plot summarizing the effect of the amino acid substitutions of Mab designs on the Tm of tested Fabs. The results are reported as the change in Fab Tm compared to WT and are shown for all the designs for which Tm was measured (“All”), and separated by paratope.

FIG. 14 depicts a min-max box plot summarizing the effect of the amino acid substitutions of Mab designs on the affinity of tested Fabs for its antigen. The results are reported as the difference in log(KD) of the appropriate Fab of the bispecific from WT (−(log(KD_variant)−log(KD_wt)). The results are shown for all designs for which affinity was measured (“All”), and separated by paratope.

FIGS. 15A-15G depict UPLC-SEC profiles of protein-A and prep-SEC purified bispecific antibodies and parent antibodies. FIG. 15A shows wild-type parent CAT-2200 mAb; FIG. 15B shows wild-type parent CR8071 mAb; FIG. 15C shows wild-type parent SGN-CD19a mAb; FIG. 15D shows wild-type parent Pertuzumab mAb; FIG. 15E shows the bispecific antibody generated using design 3972 CAT-2200/Pertuzumab SMCA; FIG. 15F shows the bispecific antibody generated using design 3972 CAT-2200/SGN-CD19a SMCA; and FIG. 15G shows the bispecific antibody generated using design 3972 CR8071/SGN-CD19a SMCA.

FIGS. 16A-16B depict the process for selecting wild-type reference values for the calculation of ‘change in total pairing with respect to WT’ and ‘change in total bispecific with respect to WT’, for designs tested in SMCA, in cases where the corresponding WT bispecific construct had not been assessed by SMCA. FIG. 16A shows the process for selecting wild-type reference values for ‘total pairing’ (“% H1L1 and % H2L2 Pairing”) for each of the three bispecific systems; FIG. 16B shows the process for selecting wild-type reference values for ‘total bispecific’ (“H1L1_H2L2 and H1L2_H2L1**”) for each of the three bispecific systems.

### DETAILED DESCRIPTION

Provided herein are engineered antibodies (also referred to herein as multispecific antigen-binding polypeptide constructs) which can comprise first heterodimer (H1L1) having a first immunoglobulin heavy chain (H1) and an immunoglobulin lambda light chain (L1) which pair to form a first Fab region, and a second heterodimer (H2L2) having an immunoglobulin heavy chain (H2) and an immunoglobulin kappa light chain (L2) which pair to form a second Fab region. The first Fab region typically binds to a first antigen and the second Fab region typically binds to a second antigen. In some embodiments, the first and second antigens are different from each other. H1 is distinct from H2. One or more of the immunoglobulin heavy chains and light chains are engineered to comprise amino acid modifications that promote preferential pairing of correctly paired heavy and light chains (H1L1 or H2L2) when co-expressed or co-produced. More specifically, the amino acid modifications promote preferential pairing between each heavy chain and the correct light chain such that the heavy chain of the first heterodimer (H1) can preferentially pair with L1 rather than L2, and the heavy chain of the second heterodimer (H2) can preferentially pair with L2 rather than L1. As a result, co-expression of H1, L1, H2 and L2 polypeptides can enable the production of correctly paired bispecific antibody with reduced or limited mispairing, thus decreasing the number and amount of mispaired species produced and potentially improving manufacturability. In one embodiment, the amino acid modifications in the Fab regions are paired with amino acid modifications in the Fc region that promote formation of a heterodimeric Fc region to further reduce the amount of mispaired heavy chains. The amino acid modifications do not significantly affect the thermal stability of the correctly paired heterodimers, or the binding affinity of each correctly paired heterodimer for antigen as compared to heterodimers that are formed from wild type H1 and L1, or H2 and L2 polypeptides.

Also provided herein are methods of making the multispecific antigen-binding polypeptide constructs described above.

### Definitions

Unless defined otherwise, all technical and scientific terms used herein have the same meaning as is commonly understood by one of skill in the art to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±1%, 2%, 3%, 4%, 5%, 6%, 7%, 8%, 9% or 10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously. In addition, it should be understood that the individual single chain polypeptides or immunoglobulin constructs derived from various combinations of the structures and substituents described herein are disclosed by the present application to the same extent as if each single chain polypeptide or heterodimer were set forth individually. Thus, selection of particular components to form individual single chain polypeptides or heterodimers is within the scope of the present disclosure.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

In the present application, amino acid names and atom names (e.g. N, O, C, etc.) are used as defined by the Protein DataBank (PDB) (www.pdb.org), which is based on the IUPAC nomenclature (IUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985). The term “amino acid residue” is primarily intended to indicate an amino acid residue contained in the group consisting of the 20 naturally occurring amino acids, i.e. alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “nucleotide sequence” or “nucleic acid sequence” is intended to indicate a consecutive stretch of two or more nucleotide molecules. The nucleotide sequence can be of genomic, cDNA, RNA, semisynthetic or synthetic origin, or any combination thereof.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing a nucleic acid sequence into a cell.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refer to compounds that have the same basic chemical structure as a naturally occurring amino acid, i.e., a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as alanine, ornithine, etc.; and chemically synthesized compounds having properties known in the art to be characteristic of amino acids. Examples of non-naturally occurring amino acids include, but are not limited to, N-methyl amino acids (e.g. methyl alanine), D-amino acids, histidine-like amino acids (e.g., 2-amino-histidine, hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins of the antigen-binding polypeptide constructs described herein can be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids are referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles of the invention.

Conservative substitution tables providing functionally similar amino acids are known to those of ordinary skill in the art. The following eight groups each contain amino acids that may be considered conservative substitutions for one another:
Alanine (A), Glycine (G);
Aspartic acid (D), Glutamic acid (E);
Asparagine (N), Glutamine (Q);
Arginine (R), Lysine (K);
Isoleucine (I), Leucine (L), Methionine (M), Valine (V);
Phenylalanine (F), Tyrosine (Y), Tryptophan (W); and
Serine(S), Threonine (T);
(see, e.g., Creighton, Proteins: Structures and Molecular Properties (W H Freeman & Co.; 2nd edition (December 1993).

The terms “identical” or percent “identity,” in the context of two or more nucleic acids or polypeptide sequences, refer to two or more sequences or subsequences that are the same. Sequences are “substantially identical” or “substantially similar” if they have a percentage of amino acid residues or nucleotides that are the same (i.e., at least about 50%, 55%, 60%, 65%, 70%, 75%, 80%, 85%, 90%, 91%, 92%, 93%, 94%, 95%, 96%, 97%, 98%, or 99% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide of the antigen-binding polypeptide constructs described herein, including homologs from species other than human, can be obtained by a process comprising the steps of screening a library under stringent hybridization conditions with a labeled probe having a polynucleotide sequence of the antigen-binding polypeptide constructs described herein or a fragment thereof, and isolating full-length cDNA and genomic clones containing said polynucleotide sequence. Such hybridization techniques are well known to the skilled artisan.

Examples of an algorithm that is suitable for determining percent sequence identity and sequence similarity are the BLAST™ and BLAST™ 2.0 algorithms, which are described in Altschul et al. (Nuc. Acids Res. 25:3389-402, 1977), and Altschul et al. (J. Mol. Biol. 215:403-10, 1990), respectively. Software for performing BLAST™ analyses is publicly available through the National Center for Biotechnology Information (see the internet at www.ncbi.nlm.nih.gov). Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. Examples of algorithm parameters for the BLASTN program (for nucleotide sequences) are wordlength (W) of 11, an expectation (E) of 10, M=5, N=−4 and a comparison of both strands. For amino acid sequences, examples of algorithm parameters for the BLASTP program are wordlength of 3, expectation (E) of 10, and the BLOSUM62 scoring matrix (see Henikoff and Henikoff, Proc. Natl. Acad. Sci. USA 89:10915, 1989).

A derivative, or a variant of a polypeptide is said to share “homology” or be “homologous” with the peptide if the amino acid sequences of the derivative or variant has at least 50% identity over a sequence that is 100 amino acids in length from the original peptide. In certain embodiments, the derivative or variant is at least 75% the same as that of either the peptide or a fragment of the peptide having the same number of amino acid residues as the derivative. In certain embodiments, the derivative or variant is at least 85% the same as that of either the peptide or a fragment of the peptide having the same number of amino acid residues as the derivative. In certain embodiments, the amino acid sequence of the derivative is at least 90% the same as the peptide or a fragment of the peptide having the same number of amino acid residues as the derivative. In some embodiments, the amino acid sequence of the derivative is at least 95% the same as the peptide or a fragment of the peptide having the same number of amino acid residues as the derivative. In certain embodiments, the derivative or variant is at least 99% the same as that of either the peptide or a fragment of the peptide having the same number of amino acid residues as the derivative.

As used herein, an “isolated” polypeptide or construct means a construct or polypeptide that has been identified and separated and/or recovered from a component of its natural cell culture environment. Contaminant components of its natural environment are materials that would typically interfere with diagnostic or therapeutic uses for the heteromultimer, and can include enzymes, hormones, and other proteinaceous or non-proteinaceous solutes.

In certain embodiments, as used herein, “isolated” antigen-binding polypeptide constructs describe antigen-binding polypeptide constructs that have been identified and separated and/or recovered from a component of its natural cell culture environment. For example, an isolated bispecific antigen-binding polypeptide construct described herein comprises heterodimer pairs or “isolated” heterodimer pairs that comprise a heterodimer or heterodimer pair that has been identified and separated and/or recovered from a component of its natural cell culture environment. Contaminant components of its natural environment are materials that would interfere with diagnostic or therapeutic uses for the heterodimer or antigen-binding polypeptide constructs, and can include enzymes, hormones, and other proteinaceous or non-proteinaceous solutes.

The heterodimers and antigen-binding polypeptide constructs can be purified to substantial homogeneity. The phrases “substantially homogeneous”, “substantially homogeneous form” and “substantial homogeneity” are used to indicate that the correctly paired product is substantially devoid of by-products originating from undesired polypeptide combinations (e.g. homodimers or mispaired heterodimers). In the context of an LCCA design set (H1L1L2), the correctly paired product is the heterodimer comprising H1 and L1 (H1L1). In the context of an LCCA design set (H2L1L2), the correctly paired product is the heterodimer comprising H2 and L2 (H2L2). In one embodiment, in the context of a bispecific antigen-binding polypeptide construct, where H1, L1, H2, and L2 are expressed, the correctly paired product is a heterodimer pair comprising correctly paired H1L1 and H2L2 (H1L1H2L2). In some embodiments, in the context of a bispecific antigen-binding polypeptide construct, where H1, L1, H2, and L2 are expressed, the correctly paired product can comprise additional products that exhibit correct pairing in at least one Fab region such as, for example, H1L1H2L1 or H1L2H2L2, or where “half antibodies” are produced, H1L1 or H2L2. Expressed in terms of purity, in one embodiment, substantial homogeneity means that the amount of completely mispaired by-products does not exceed 20%, for example is below 10%, below 5%, below 1%, or below 0.5% of the total LC-MS intensity from all species present in the mixture, wherein the percentages reflect results from Mass Spectrometric analysis.

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al, Eds., Antibodies: A Laboratory Manual, Chapter 14 (Cold Spring Harbor Laboratory, Cold Spring Harbor, 1988).

As used herein, the terms “antibody” and “immunoglobulin” or “antigen-binding polypeptide construct” are used interchangeably. An “antigen-binding polypeptide construct” refers to a polypeptide substantially encoded by an immunoglobulin gene or immunoglobulin genes, or one or more fragments thereof, which specifically bind an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin isotypes, IgG, IgM, IgA, IgD, and IgE, respectively. Further, the antibody can belong to one of a number of subtypes, for instance, the IgG can belong to the IgG1, IgG2, IgG3, or IgG4 subclasses.

An exemplary immunoglobulin (antibody) structural unit is composed of two pairs of polypeptide chains, each pair having one immunoglobulin “light” (about 25 kD) and one immunoglobulin “heavy” chain (about 50-70 kD). This type of immunoglobulin or antibody structural unit is considered to be “naturally occurring.” The term “light chain” includes a full-length light chain and fragments thereof having sufficient variable domain sequence to confer binding specificity. A full-length light chain includes a variable domain, VL, and a constant domain, CL. The variable domain of the light chain is at the amino-terminus of the polypeptide. Light chains include kappa chains and lambda chains. The term “heavy chain” includes a full-length heavy chain and fragments thereof having sufficient variable region sequence to confer binding specificity. A full-length heavy chain includes a variable domain, VH, and three constant domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subclasses), IgA (including IgA1 and IgA2 subclasses), IgM, IgD and IgE. The term “variable region” or “variable domain” refers to a portion of the light and/or heavy chains of an antibody generally responsible for antigen recognition, typically including approximately the amino-terminal 120 to 130 amino acids in the heavy chain (VH) and about 100 to 110 amino terminal amino acids in the light chain (VL).

A “complementarity determining region” or “CDR” is an amino acid sequence that contributes to antigen-binding specificity and affinity. “Framework” regions (FR) can aid in maintaining the proper conformation of the CDRs to promote binding between the antigen-binding region and an antigen. Structurally, framework regions can be located in antibodies between CDRs. The variable regions typically exhibit the same general structure of relatively conserved framework regions (FR) joined by three hyper variable regions, CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3, and FR4. The assignment of amino acids to each domain is typically in accordance with the definitions of Kabat Sequences of Proteins of Immunological Interest (National Institutes of Health, Bethesda, Md. (1987 and 1991)), unless stated otherwise.

A “multispecific antigen-binding polypeptide construct” or “multispecific antibody” is one that targets or binds to more than one distinct antigen or epitope. A “bispecific,” “dual-specific” or “bifunctional” antigen-binding polypeptide construct or antibody is a species of multispecific antigen-binding polypeptide construct that targets or binds to two different antigens or epitopes. In general, a bispecific antigen-binding polypeptide construct can have two different antigen-binding domains. The two antigen-binding domains of a bispecific antigen-binding polypeptide construct or antibody will bind to two different epitopes, which can reside on the same or different molecular targets. In one embodiment, the bispecific antigen-binding polypeptide construct is in a naturally occurring format. In other words, the bispecific antigen-binding polypeptide construct has the same format as a naturally occurring IgG, IgA, IgM, IgD, or IgE antibody.

Antibody heavy chains pair with antibody light chains and meet or contact one another at one or more “interfaces.” An “interface” includes one or more “contact” amino acid residues in a first polypeptide that interact with one or more “contact” amino acid residues of a second polypeptide. For example, an interface exists between the two CH3 domains of a dimerized Fc region, between the CH1 domain of the heavy chain and CL domain of the light chain, and between the VH domain of the heavy chain and the VL domain of the light chain. The “interface” can be derived from an IgG antibody and for example, from a human IgG1 antibody.

The term “amino acid modifications” as used herein includes, but is not limited to, amino acid insertions, deletions, substitutions, chemical modifications, physical modifications, and rearrangements.

The amino acid residues for the immunoglobulin heavy and light chains may be numbered according to several conventions including Kabat (as described in Kabat and Wu, 1991; Kabat et al, Sequences of proteins of immunological interest. 5th Edition-US Department of Health and Human Services, NIH publication no. 91-3242, p 647 (1991)), IMGT (as set forth in Lefranc, M.-P., et al., IMGT®, the international ImMunoGeneTics information system® Nucl. Acids Res, 37, D1006-D1012 (2009), and Lefranc, M.-P., IMGT, the International ImMunoGeneTics Information System, Cold Spring Harb Protoc. 2011 Jun. 1; 2011 (6)), 1JPT (as described in Katja Faelber, Daniel Kirchhofer, Leonard Presta, Robert F Kelley, Yves A Muller, The 1.85 Å resolution crystal structures of tissue factor in complex with humanized fab d3h44 and of free humanized fab d3h44: revisiting the solvation of antigen combining sites1, Journal of Molecular Biology, Volume 313, Issue 1, Pages 83-97) and EU (according to the EU index as in Kabat referring to the numbering of the EU antibody (Edelman et al., 1969, Proc Natl Acad Sci USA 63:78-85)). Kabat numbering is used herein for the VH, CH1, CL, and VL domains unless otherwise indicated. EU numbering is used herein for the CH3 and CH2 domains, and the hinge region unless otherwise indicated. Table 22A provides a correspondence table showing the amino acid numbering for selected positions in the IgG1 heavy chain polypeptide using IMGT, Kabat, 1JPT, and EU numbering systems. Table 22B provides a correspondence table showing the amino acid numbering for selected positions in the lambda light chain polypeptide using IMGT and Kabat numbering systems. Table 22C provides a correspondence table showing the amino acid numbering for selected positions in the kappa light chain polypeptide using IMGT, 1JPT and Kabat numbering systems.

### Antigen-Binding Polypeptide Constructs

The antigen-binding polypeptide constructs (i.e. antibodies) described herein can be multispecific or bispecific. A multispecific antigen-binding polypeptide construct can comprise at least a first heterodimer (H1L1) having a first immunoglobulin heavy chain polypeptide sequence (H1) and an immunoglobulin lambda light chain polypeptide sequence (L1) that form a first Fab region and at least a second heterodimer (H2L2) having an immunoglobulin heavy chain polypeptide sequence (H2) and an immunoglobulin kappa light chain polypeptide sequence (L2) that form a second Fab region, where H1 and H2 are distinct from each other. In one embodiment, the bispecific antigen-binding polypeptide construct comprises a first heterodimer (H1L1) having a first immunoglobulin heavy chain polypeptide sequence (H1) and an immunoglobulin lambda light chain polypeptide sequence (L1) that form a first Fab region and a second heterodimer (H2L2) having an immunoglobulin heavy chain polypeptide sequence (H2) and an immunoglobulin kappa light chain polypeptide sequence (L2) that form a second Fab region, where H1 and H2 are distinct from each other. In one embodiment, each heterodimer comprises a single Fab region. The term “Fab region” as used herein refers to the region resulting from the pairing of one immunoglobulin light chain polypeptide sequence with a one immunoglobulin heavy chain polypeptide sequence, and is composed of the VH and CH1 domains of the immunoglobulin heavy chain polypeptide sequence, and the VL and CL domains of the immunoglobulin light chain polypeptide sequence. In some embodiments, the first Fab region binds to a first antigen, and the second Fab region binds to a second antigen. The first and second antigens can be the same or different. One or more of the immunoglobulin heavy chains and light chains can comprise amino acid modifications that promote preferential pairing of correctly paired heavy and light chains when co-expressed or co-produced.

When the antigen-binding polypeptide construct is a bispecific antigen-binding polypeptide construct (i.e. bispecific antibody), it can also be referred to as a “heterodimer pair.”

For the sake of illustration, the first heterodimer of the antigen-binding polypeptide construct is referred to as H1L1, and comprises a first immunoglobulin heavy chain polypeptide sequence (H1) paired with an immunoglobulin lambda light chain polypeptide sequence (L1), and the second heterodimer is referred to as H2L2 and comprises a second immunoglobulin heavy chain polypeptide sequence (H2) paired with an immunoglobulin kappa light chain polypeptide sequence (L2). It should be understood, however, that this designation is arbitrary and meant only to specify that one heterodimer comprises an immunoglobulin kappa light chain and the other comprises an immunoglobulin lambda light chain. The Fab region of the first heterodimer, H1L1, may also be referred to herein as the “lambda Fab”; while the Fab region of the second heterodimer, H2L2, may also be referred to herein as the “kappa Fab.”

### Parent Antibodies

The immunoglobulin heavy chain polypeptide sequences, also referred to as “heavy chains,” and immunoglobulin light chain polypeptide sequences, also referred to as “light chains,” of each heterodimer can be obtained from one or more parent antibodies, where at least one parent antibody comprises a kappa light chain, and at least one other parent antibody comprises a lambda light chain, and the amino acid modifications that promote preferential pairing are engineered into these heavy and light chains. The parent immunoglobulin heavy chain and immunoglobulin light chain sequences lacking the amino acid modifications that promote preferential pairing are referred to as wild type immunoglobulin heavy chain polypeptide sequences, wild type immunoglobulin kappa light chain polypeptide sequences, and wild type immunoglobulin lambda light chain polypeptides sequences. In one embodiment, the heavy and light chains of the heterodimers of the antigen-binding polypeptide construct are obtained from two parent antibodies. Generally, the two parent antibodies are different from each other; however, this is not necessarily always the case. In one embodiment, the antigen-binding polypeptide construct is a bispecific antigen-binding polypeptide construct where each heterodimer is obtained from a different parent antibody. In another embodiment, the antigen-binding polypeptide construct is a bispecific antigen-binding polypeptide construct where both parent antibodies bind to the same antigen, but target different epitopes on the same antigen. In one embodiment, at least one parent antibody is monospecific, i.e. can bind to only one epitope. In another embodiment, at least one parent antibody can bind to more than one epitope.

The heavy chain and light chain of each heterodimer of the antigen-binding polypeptide construct pair to form a Fab region that specifically binds to the same antigen as the parent antibody they were obtained from. For example, if a bispecific antigen-binding polypeptide construct was prepared based on parent antibodies CAT-2200 (containing a lambda light chain, and binding to IL-17A) and D3H44 (containing a kappa light chain, and binding to tissue factor), the heavy chain and light chain of one heterodimer would pair to form a Fab region that binds to IL-17A, and the heavy and light chain of the second heterodimer would pair to form a Fab region that binds to tissue factor.

The parent antibodies can be obtained from species including, but not limited to humans, mice, rats, rabbits, sheep, cows, goats or camels. In one embodiment, the parent antibodies can be obtained from humans or mice.

Parent antibodies can also include those that are prepared from hybridomas using standard monoclonal antibody production protocols, such as those described by Kohler and Milstein (Nature, 256:495-497, 1975).

Antibodies binding to a particular target may also be identified by a number of different strategies, including phage display, in vitro display, and other methods. These strategies result in antibodies that are in scFv format, Fab format or full-length IgG format. A review of these strategies are found in Chapter 4 of Therapeutic Antibody Engineering, by William R. Strohl and Lila M. Strohl, Woodhead Publishing series in Biomedicine No 11, ISBN 1 907568 37 9, October 2012. In one embodiment, parent antibodies include antibodies identified by phage display or in vitro display. Antibodies identified in formats other than Fab or full-length IgG formats can be converted into same as known in the art. Methods of converting scFvs to Fabs are well known in the art (see for example, Steinwand et al. Mabs 6:204-218, or Zuberbuhler et al. Protein Engineering, Design & Selection 22:169-174). In one embodiment, antibodies originally identified as scFvs, but where the scFv has been converted to Fab format and engineered into the form of a conventional or naturally occurring antibody may also be used as parent antibodies.

In one embodiment, the heavy chains and light chains of each heterodimer of the antigen-binding polypeptide construct can be obtained from a parent antibody that is a humanized antibody. Humanized antibodies can be obtained by substituting the complementarity determining region (CDR) of a human antibody for the CDR of an antibody derived from a nonhuman mammal, for example, a mouse. Methods for identifying CDRs are known in the art (Kabat et al., Sequence of Proteins of Immunological Interest (1987), National Institute of Health, Bethesda, Md.; Chothia et al., Nature (1989) 342:877). General genetic recombination techniques suitable for this purpose are also known (see European Patent Application Publication No. EP 125023; and WO 96/02576). For example, the CDR of a mouse antibody can be determined by known methods, and a DNA can be prepared such that it encodes an antibody in which the CDR is ligated with the framework region (FR) of a human antibody. A humanized antibody can then be produced using a system that uses conventional expression vectors. Such DNAs can be synthesized by PCR, using as primers several oligonucleotides designed to include portions that overlap the ends of both the CDR and FR regions (see the method described in WO 98/13388). Human antibody FRs linked via CDRs are selected such that the CDRs form a suitable antigen-binding site. If required, amino acids in the FRs of an antibody variable region may be modified so that the CDRs of the reshaped human antibody can form a suitable antigen-binding domain (Sato, K. et al., Cancer Res. (1993) 53:851-856). Modifiable amino acid residues in the FRs include portions that directly bind to an antigen via non-covalent bonds (Amit et al., Science (1986) 233:747-53), portions that have some impact or effect on the CDR structure (Chothia et al., J. Mol. Biol. (1987) 196:901-17), and portions involved in the interaction between VH and VL (EP 239400).

In one embodiment, the heavy chains and light chains of each heterodimer of the antigen-binding polypeptide construct can be obtained from a parent antibody that is a chimeric antibody. Chimeric antibodies are antibodies prepared by combining sequences derived from different animals. For example, a chimeric antibody can be produced by combining the heavy chain and light chain variable domains from a mouse antibody with the heavy chain and light chain constant domains from a human antibody. Chimeric antibodies can be prepared by known methods. To obtain such chimeric antibodies, for example, a DNA encoding an antibody variable domain may be ligated with a nucleic acid encoding a human antibody constant domain; the resulting ligation product can be inserted into an expression vector; and the construct can be introduced into a host cell to produce the chimeric antibody.

The heavy and light chains of the heterodimers can be obtained from numerous parent antibodies known in the art. Most antibodies can serve as a parent antibody, provided that they comprise an immunoglobulin heavy chain polypeptide sequence that pairs with an immunoglobulin light chain polypeptide sequence to form Fab region that binds to an antigen. In one embodiment, at least one parent antibody is a therapeutic antibody i.e. an antibody that is used to treat a disease. Non-limiting examples of suitable therapeutic antibodies comprising a kappa light chain and the antigens they bind to are identified in Table A below:

TABLE A
Exemplary therapeutic antibodies comprising a kappa light chain

| Antibody | Antigens |
|---|---|
| ABI793 | CD40 Ligand |
| ABT-806 | Epidermal growth factor receptor |
| AV-203 | Receptor tyrosine-protein kinase erbB-3 |
| BTI-322 | CD2 |
| Bioanalytica patent | Heat shock protein 90 homolog |
| anti-HSP90 | |
| CTM01 | Mucin 1 |
| Centocor patent | Glucagon-like peptide 1 Receptor |
| anti-GLP-1R | |
| Chugai AHM | Bone marrow stromal antigen 2 |
| DX-2930 | Kallikrein |
| Genmab patent | B-lymphocyte antigen CD20 |
| anti-CD20 | |
| HuMax-CD32b | Fc-gamma-RIIB (CD32b) |
| HuMax-IL8 | Interleukin 8 |
| IMA-026 | Interleukin 13 |
| IMMU-114 | HLA-DR |
| Immunomedics hA19 | CD19 |
| Immunomedics patent | Insulin-like Growth Factor 1 Receptor |
| anti-IGF-1R | |
| MDX-1303 | Anthrax Protective antigen |
| MDX-1401 | CD30 |
| MEDI0639 | Delta like ligand 4 |
| MEDI4893 | Staphylococcus aureus alpha toxin |
| MGA271 | B7-H3 |
| MGAWN1 | West Nile Virus Envelope Protein |
| MH166 | Interleukin 6 |
| MT293 | Collagen |
| Macrogenics patent | KID3 |
| anti-KID3 | |
| OPR-003 | Interleukin 6 |
| Stem Centrx patent | Cadherin-1 |
| anti-Cadherin-1 | |
| abagovomab | Antibody against Cancer antigen 125 |
| abituzumab | Integrin alpha-V |
| abrilumab | Integrin alpha4 beta7 |
| actoxumab | C. difficile Toxin A |
| adalimumab | Tumor necrosis factor-alpha |
| adecatumumab | Epithelial cell adhesion molecule |
| aducanumab | Amyloid beta |
| afasevikumab | Interleukin 17A, Interleukin 17F |
| Alemtuzumab | CD52 |
| Alirocumab | Proprotein Convertase Subtilisin/Kexin Type 9 |
| Amatuximab | Mesothelin |
| Anifrolumab | Interferon alpha receptor 1 |
| Anrukinzumab | Interleukin 13 |
| Ascrinvacumab | Activin receptor like kinase 1 |
| Atezolizumab | Programmed Cell Death 1 Ligand 1 |
| Atinumab | Nogo-A |
| Basiliximab | Interleukin-2 receptor alpha chain |
| Bavituximab | Phosphatidylserine |
| Begelomab | CD26 |
| Benralizumab | Interleukin 5 receptor subunit alpha |
| Bertilimumab | Chemokine (C-C motif) ligand 11 |
| besilesomab | CEA (carcinoembryonic antigen)-related antigen |
| bezlotoxumab | C. difficile Toxin B |
| bimekizumab | Interleukin 17A, Interleukin 17F |
| bleselumab | CD40 |
| blosozumab | Sclerostin |
| bococizumab | Proprotein Convertase Subtilisin/Kexin Type 9 |
| brodalumab | Interleukin 17 Receptor Alpha |
| canakinumab | Interleukin 1, beta |
| carlumab | Monocyte chemoattractant protein-1 |
| cetuximab | Epidermal growth factor receptor |
| clazakizumab | Interleukin 6 |
| codrituzumab | Glypican 3 |
| conatumumab | Cytokine Death Receptor 5 |
| concizumab | Tissue factor pathway inhibitor |
| crenezumab | Amyloid beta |
| dacetuzumab | CD40 |
| daclizumab | Interleukin-2 receptor alpha chain |
| dalotuzumab | Insulin-like Growth Factor 1 Receptor |
| daratumumab | CD38 (ADP-ribosyl cyclase 1) |
| dectrekumab | Interleukin 13 |
| demcizumab | Delta like ligand 4 |
| dinutuximab | GD2 ganglioside |
| dupilumab | Interleukin 4 Receptor Alpha |
| durvalumab | Programmed Cell Death 1 Ligand 1 |
| ecromeximab | GD3 Ganglioside |
| efalizumab | Integrin Î ± L |
| eldelumab | Interferon Gamma-inducible Protein (IP-10) |
| elgemtumab | Receptor tyrosine-protein kinase erbB-3 |
| elotuzumab | SLAM family member 7 |
| elsilimomab | Interleukin 6 |
| emactuzumab | CSF-1 Receptor |
| emibetuzumab | cMet Receptor |
| enavatuzumab | TWEAK Receptor |
| enokizumab | Interluekin 9 |
| enoticumab | Delta like ligand 4 |
| ensituximab | Mucin 5AC |
| etaracizumab | Integrin alphaV beta3 |
| etrolizumab | Integrin alpha4 beta7 |
| evinacumab | Angiopoietin-like protein 3 |
| farletuzumab | Folate Receptor Alpha |
| fasinumab | Nerve growth factor |
| fibatuzumab | EphA3 receptor tyrosine kinase |
| ficlatuzumab | Hepatocyte growth/scatter factor |
| figitumumab | Insulin-like Growth Factor 1 Receptor |
| flanvotumab | Tyrosinase-related protein 1 |
| fletikumab | Interleukin 20 |
| foralumab | T-cell surface glycoprotein CD3 epsilon chain |
| foravirumab | Rabies virus glycoprotein |
| fresolimumab | Transforming growth factor beta |
| fulranumab | Nerve growth factor |
| ganitumab | Insulin-like Growth Factor 1 Receptor |
| gantenerumab | Amyloid beta |
| gevokizumab | Interleukin 1, beta |
| girentuximab | Carbonic anhydrase IX |
| golimumab | Tumor necrosis factor-alpha |
| ibalizumab | CD4 |
| icrucumab | FMS-like tyrosine kinase-1 |
| imalumab | Macrophage migration inhibitory factor |
| imgatuzumab | Epidermal growth factor receptor |
| inclacumab | P-selectin |
| indusatumab | Guanylyl cyclase C |
| inebilizumab | CD19 |
| infliximab | Tumor necrosis factor-alpha |
| intetumumab | Integrin alpha-V |
| ipilimumab | Cytotoxic T-Lymphocyte Antigen 4 |
| isatuximab | CD38 (ADP-ribosyl cyclase 1) |
| itolizumab | CD6 |
| ixekizumab | Interleukin 17A |
| keliximab | CD4 |
| labetuzumab | Carcinoembryonic antigen |
| landogrozumab | Myostatin |
| lebrikizumab | Interleukin 13 |
| lemalesomab | NCA-90 granulocyte cell antigen |
| lenzilumab | Granulocyte Macrophage Colony |
| | Stimulating Factor |
| libivirumab | Hepatitis B virus surface antigen |
| ligelizumab | Immunoglobulin E |
| lilotomab | Leukocyte antigen CD37 |
| lirilumab | KIR Inhibitory Receptor |
| lodelcizumab | Proprotein Convertase Subtilisin/Kexin Type 9 |
| lucatumumab | CD40 |
| lumiliximab | CD23-low affinity IgE Fc receptor |
| lumretuzumab | Receptor tyrosine-protein kinase erbB-3 |
| margetuximab | Human Epidermal growth factor Receptor 2 |
| matuzumab | Epidermal growth factor receptor |
| mepolizumab | Interleukin 5 |
| metelimumab | Transforming growth factor beta 1 |
| milatuzumab | Major histocompatibility complex |
| mogamulizumab | C—C chemokine receptor type 4 |
| monalizumab | NKG2A |
| motavizumab | Respiratory Syncytial Virus F protein |
| namilumab | Granulocyte Macrophage Colony |
| | Stimulating Factor |
| natalizumab | Integrin Alpha 4 |
| necitumumab | Epidermal growth factor receptor |
| nemolizumab | Interleukin 31 receptor subunit alpha |
| nesvacumab | Angiopoietin 2 |
| nimotuzumab | Epidermal growth factor receptor |
| nivolumab | Programmed Cell Death 1 |
| obinutuzumab | B-lymphocyte antigen CD20 |
| ocaratuzumab | B-lymphocyte antigen CD20 |
| ocrelizumab | B-lymphocyte antigen CD20 |
| ofatumumab | B-lymphocyte antigen CD20 |
| olaratumab | Platelet-derived Growth Factor Receptor |
| | Alpha subunit |
| olokizumab | Interleukin 6 |
| omalizumab | Immunoglobulin E |
| ontuxizumab | Endosialin |
| opicinumab | LRR and Ig domain ′containing, Nogo |
| | receptor′ interacting protein |
| oregovomab | Cancer antigen 125 |
| oxelumab | OX40 Ligand |
| ozanezumab | Nogo-A |
| pagibaximab | Staphylococcus epidermidis lipoteichoic acid |
| palivizumab | Respiratory Syncytial Virus F protein |
| pamrevlumab | Connective Tissue Growth Factor |
| panitumumab | Epidermal growth factor receptor |
| panobacumab | Pseudomonas aeruginosa serotype IATS O11 |
| parsatuzumab | Epidermal Growth Factor Domain-Like 7 |
| pascolizumab | Interleukin 4 |
| pateclizumab | Lymphotoxin alpha |
| patritumab | Receptor tyrosine-protein kinase erbB-3 |
| pembrolizumab | Programmed Cell Death 1 |
| perakizumab | Interleukin 17A |
| pertuzumab | Human Epidermal growth factor Receptor 2 |
| pidilizumab | Programmed Cell Death 1 |
| plozalizumab | C—C chemokine receptor type 2 |
| ponezumab | Amyloid beta |
| pritoxaximab | Shiga toxin (E. coli) |
| pritumumab | Vimentin |
| quilizumab | Immunoglobulin EM1 prime |
| racotumomab | Ganglioside GM3 |
| ralpancizumab | Proprotein Convertase Subtilisin/Kexin Type 9 |
| ramucirumab | VEGFR-2 Kinase insert domain receptor |
| rilotumumab | Hepatocyte growth/scatter factor |
| rinucumab | Platelet-derived growth factor receptor Beta |
| risankizumab | Interleukin 23 p19 |
| rituximab | B-lymphocyte antigen CD20 |
| robatumumab | Insulin-like Growth Factor 1 Receptor |
| roledumab | Rh blood group, D antigen |
| romosozumab | Sclerostin |
| rontalizumab | Interferon alpha |
| rovalpituzumab | DLL3 |
| ruplizumab | CD40 Ligand |
| samalizumab | CD200 |
| sarilumab | Interleukin-6 receptor |
| secukinumab | Interleukin 17A |
| setoxaximab | Shiga toxin (E. coli) |
| sifalimumab | Interferon alpha |
| siltuximab | Interleukin 6 |
| simtuzumab | Lysyl oxidase-like-2 |
| sirukumab | Interleukin 6 |
| sofituzumab | Cancer antigen 125 |
| solanezumab | Amyloid beta |
| sontuzumab | Mucin 1 |
| suvizumab | HIV-1 envelope glycoprotein gp120 third |
| | variable loop V3 |
| tabalumab | B-cell activating factor |
| talizumab | Immunoglobulin E |
| tanezumab | Nerve growth factor |
| tarextumab | Notch2, Notch3 |
| tefibazumab | Staphylococcus aureus Clumping factor A |
| tenatumomab | Tenascin C |
| teplizumab | T-cell surface glycoprotein CD3 epsilon chain |
| teprotumumab | Insulin-like Growth Factor 1 Receptor |
| tigatuzumab | Cytokine Death Receptor 5 |
| tildrakizumab | Interleukin 23 p19 |
| tocilizumab | Interleukin-6 receptor |
| toralizumab | CD40 Ligand |
| tovetumab | Platelet-derived Growth Factor Receptor |
| | Alpha subunit |
| trastuzumab | Human Epidermal growth factor Receptor 2 |
| tregalizumab | CD4 |
| tremelimumab | Cytotoxic T-Lymphocyte Antigen 4 |
| trevogrumab | Myostatin |
| ublituximab | B-lymphocyte antigen CD20 |
| ulocuplumab | Chemokine receptor CXCR4 |
| urelumab | 4-IBB ligand receptor |
| urtoxazumab | Escherichia coli Shiga-like toxin II B subunit |
| vapaliximab | Vascular Adhesion Protein VAP-1 |
| varlilumab | CD27 |
| vatelizumab | Integrin Alpha 2 Beta 1 |
| vedolizumab | Integrin alpha4 beta7 |
| veltuzumab | B-lymphocyte antigen CD20 |
| vesencumab | Neuropilin-1 |
| visilizumab | CD3 T-Cell Co-Receptor |
| vorsetuzumab | CD70 |
| zalutumumab | Epidermal growth factor receptor |
| zanolimumab | CD4 |
| zatuximab | Epidermal growth factor receptor |

Non-limiting examples of suitable therapeutic antibodies comprising a lambda light chain and the antigens they bind to are identified in Table B below:

TABLE B
Exemplary therapeutic antibodies comprising a lambda light chain

| Antibody | Antigens |
|---|---|
| avelumab | Programmed Cell Death 1 Ligand 1 |
| belimumab | B-cell activating factor |
| bimagrumab | activin receptor MB |
| briakinumab | Interleukin 12 |
| brontictuzumab | Notch1 |
| cixutumumab | Insulin-like Growth Factor 1 Receptor |
| drozitumab | Cytokine Death Receptor 5 |
| evolocumab | Proprotein Convertase Subtilisin/Kexin Type 9 |
| exbivirumab | Hepatitis B virus surface antigen |
| fezakinumab | Interleukin 22 |
| galiximab | CD80 |
| guselkumab | Interleukin 23 p19 |
| lexatumumab | Cytokine Death Receptor 5 |
| mapatumumab | TRAIL Receptor-1 |
| mavrilimumab | Granulocyte-macrophage colony-stimulating |
| | factor receptor subunit alpha |
| namatumab | Ron Receptor |
| orticumab | Oxidized LDL |
| otelixizumab | CD3 T-Cell Co-Receptor |
| rafivirumab | Rabies virus glycoprotein |
| raxibacumab | Anthrax Protective antigen |
| seribantumab | Receptor tyrosine-protein kinase erbB-3 |
| tesidolumab | Complement component 5 |
| tezepelumab | Thymic Stromal Lymphopoietin |
| tralokinumab | Interleukin 13 |
| vantictumab | Frizzled receptor |

### Immunoglobulin Subclasses

Immunoglobulin heavy chains of the parent antibodies fall within the following classes: IgA1, IgA2, IgM, IgD, IgE, IgG1, IgG2, IgG3, and IgG4. In one embodiment, the first and second heterodimer of the antigen-binding polypeptide construct comprises an IgG heavy chain. In one embodiment, the first and second heterodimer of the antigen-binding polypeptide construct comprises an IgG1 heavy chain. The immunoglobulin light chains of the parent antibodies are either kappa light chains or lambda light chains.

The antigen-binding polypeptide constructs described herein comprise at least one heterodimer having an immunoglobulin heavy chain polypeptide sequence and an immunoglobulin kappa light chain polypeptide sequence, and at least another heterodimer having an immunoglobulin heavy chain polypeptide sequence and an immunoglobulin lambda light chain polypeptide sequence. In one embodiment, the antigen-binding polypeptide construct comprises one heterodimer having an IgG heavy chain polypeptide sequence and an immunoglobulin kappa light chain polypeptide sequence, and another heterodimer having an IgG heavy chain polypeptide sequence and an immunoglobulin lambda light chain polypeptide sequence.

In one embodiment, the antigen-binding polypeptide construct comprises a heterodimer having a heavy chain with a VH domain selected from the VH domain germline groups IGHV1, IGHV2, IGHV3, IGHV4, IGHV5, IGHV6 or IGHV7. In one embodiment, the antigen-binding polypeptide construct comprises a heterodimer having a heavy chain with a VH domain from germline subgroup IGHV3. In another embodiment, the antigen-binding polypeptide construct comprises a heterodimer having a heavy chain with a J segment selected from the J segment germline genes IGHJ1, IGHJ2, IGHJ3, IGHJ4, IGHJ5, or IGHJ6. In another embodiment, the antigen-binding polypeptide construct comprises a heterodimer having a heavy chain with a J segment from germline subgroup IGHJ4. In one embodiment, the antigen-binding polypeptide construct comprises a heterodimer having a heavy chain with a CH1 domain selected from the CH1 domain germline subgroups IGHG1, IGHG2, IGHG3, or IGHG4. In one embodiment, the antigen-binding polypeptide construct comprises a heterodimer having a heavy chain with a CH1 domain from germline subgroup IGHG1.

For heterodimers comprising a lambda light chain polypeptide sequence, the lambda light chains can comprise a CL-lambda domain selected from the germline genes IGLC1, IGLC2, IGLC3, IGLC6, or IGLC7. In one embodiment, the antigen-binding polypeptide construct comprises a heterodimer having a lambda light chain comprising a CL-lambda domain from germline subgroup IGLC2. The lambda light chains can comprise a VL-lambda domain selected from the germline subgroups IGLV1, IGLV2, IGLV3, IGLV4, IGLV5, IGLV6, IGLV7, IGLV8, IGLV9, IGLV10 or IGLV11. In one embodiment, the antigen-binding polypeptide construct comprises a heterodimer with a lambda light chain having a VL-lambda domain from the germline subgroup IGLV6. In another embodiment, the antigen-binding polypeptide construct comprises a heterodimer having lambda light chain with a lambda J segment selected from the J segment germline genes IGLJ1, IGLJ2, IGLJ3, IGLJ6 or IGLJ7. In another embodiment, the antigen-binding polypeptide construct comprises a heterodimer having a heavy chain with a lambda J segment from germline subgroup IGLJ2.

For heterodimers comprising a kappa light chain polypeptide sequence, the kappa light chains can comprise a CL-kappa domain selected from the CL germline alleles IGKC*01, IGKC*02, IGKC*03, IGKC*04, or IGKC*05. In one embodiment, the antigen-binding polypeptide construct comprises a heterodimer having a kappa light chain comprising a CL-kappa domain from germline subgroup IGKC*01. The kappa light chains can comprise a VL-kappa domain selected from the germline subgroups IGKV1, IGKV1D, IGKV2, IGKV3, IGKV4, IGKV5, or IGKV6. In one embodiment, the antigen-binding polypeptide construct comprises a heterodimer with a kappa light chain having a VL-kappa domain from the germline subgroup IGKV1. In another embodiment, the antigen-binding polypeptide construct comprises a heterodimer having kappa light chain with a J segment selected from the J segment germline genes IGKJ1, IGKJ2, IGKJ3, IGKJ4 or IGKJ5. In another embodiment, the antigen-binding polypeptide construct comprises a heterodimer having a kappa light chain with a J segment from germline subgroup IGKJ1 or IGKJ2.

Immunoglobulin heavy chains typically comprise at least one variable (VH) domain, and three constant domains, CH1, CH2, and CH3. In one embodiment, each heavy chain of the first heterodimer and second heterodimer of the antigen-binding polypeptide construct comprises a VH domain, a CH1 domain, a CH2 domain, and a CH3 domain. In another embodiment, each heavy chain of the first heterodimer and second heterodimer comprises a VH domain, a CH1 domain, and a CH3 domain. In still another embodiment, each heavy chain of the first heterodimer and second heterodimer comprises a VH domain and a CH1 domain. Immunoglobulin light chains typically comprise one variable (VL) domain and one constant (CL) domain. In one embodiment, the light chain of each heterodimer comprises a VL domain and a CL domain.

As indicated above, in some embodiments, the immunoglobulin heavy chain polypeptide sequence and the immunoglobulin light chain polypeptide sequence of each heterodimer can be obtained from a known therapeutic antibody, or from an antibody that binds various target molecules or cancer antigens. The amino acid and nucleotide sequences of numerous such molecules are readily available (see for example, GenBank Accession No: AJ308087.1 (Humanized anti-human tissue factor antibody D3H44 light chain variable region and CL domain); GenBank® Accession No: AJ308086.1 (humanized anti-human tissue factor antibody D3H44 heavy chain variable region and CH1 domain); GenBank® Accession No: HC359025.1 (Pertuzumab Fab light chain gene module); GenBank® Accession No: HC359024.1 (Pertuzumab Fab heavy chain gene module); GenBank® Accession No: GM685465.1 (Antibody Trastuzumab (=Herceptin)-wildtype; light chain); GenBank® Accession No: GM685463.1 (Antibody Trastuzumab (=Herceptin®)-wildtype; heavy chain); GenBank® Accession No: GM685466.1 (Antibody Trastuzumab (=Herceptin®)-GC-optimized light chain); and GenBank® Accession No: GM685464.1 (Antibody Trastuzumab (=Herceptin®)-GC-optimized heavy chain. The sequences of each of the polypeptides described above are available from the NCBI website as of Nov. 28, 2012 and are each incorporated by reference in its entirety for all purposes. Amino acid and nucleotide sequences for cetuximab are also known in the art, see for example the Drug Bank website supported by Canadian Institutes of Health Research, Alberta Innovates-Health Solutions, and by The Metabolomics Innovation Centre (TMIC), Accession No. DB00002.

Amino Acid Modifications that Promote Preferential Pairing

One or more of the heavy and light chains H1, L1, H2, and L2 comprise amino acid modifications that promote preferential pairing between heavy and light chains, that are engineered into the heavy and light chains of the parent antibodies. In one embodiment, two of the heavy and light chains H1, L1, H2, and L2 comprise amino acid modifications that promote preferential pairing between heavy and light chains. In one embodiment, three of the heavy and light chains H1, L1, H2, and L2 comprise amino acid modifications that promote preferential pairing between heavy and light chains.

In some embodiments, the amino acid modifications can be asymmetric, in that the amino acid positions that are modified are different between H1 and H2, and between L1 and L2.

In one embodiment, H2 and L2 comprise amino acid modifications that promote preferential pairing between heavy and light chains, while H1 and L1 do not. In one embodiment, H1, L1, and H2 comprise amino acid modifications that promote preferential pairing between heavy and light chains, while L2 does not. In one embodiment, H1, H2, and L2 comprise amino acid modifications that promote preferential pairing between heavy and light chains, while L1 does not. In one embodiment, L1, H2 and L2 comprise amino acid modifications that promote preferential pairing between heavy and light chains, while H1 does not.

In one embodiment, the one or more amino acid modifications comprise one or more amino acid substitutions. The amino acid modifications promote preferential pairing of H1 with L1 and H2 with L2 when H1 or H2 are co-expressed with L1 and L2, or when H1, L1, H2, and L2 are co-expressed. As indicated above, for the sake of illustration, the heterodimers of the antigen-binding polypeptide construct will be identified as follows: the HILL heterodimer comprises a lambda light chain L1, and the H2L2 heterodimer comprises a kappa light chain L2.

A “Mab design,” or “Mab design set” as used herein refers to a specific set of amino acid modifications that promote preferential pairing that are present in one set of H1, L1, H2 and L2, and is also identified as H1L1H2L2. Amino acid modifications in one or more of H1, L1, H2, and L2 that promote preferential pairing are referred to and presented as Mab designs or Mab design sets (i.e. H1L1H2L2). The Mab design sets are initially tested as LCCA design sets (i.e. H1L1L2 or H2L1L2) to determine the strength of pairing specificity, where H1 and H2 are individually co-expressed with L1 and L2.

In one embodiment, the amino acid modifications can be made to one or more amino acids that are part of the interface between the light chain and heavy chain. In one embodiment, the amino acid modifications introduced in the immunoglobulin heavy chain polypeptide sequences and immunoglobulin light chain polypeptide sequences are complementary to each other. Complementarity at the heavy and light chain interface can be achieved on the basis of steric and hydrophobic contacts, electrostatic/charge interactions or a combination of these and a variety of other interactions. The complementarity between protein surfaces is broadly described in the literature in terms of lock and key fit, knob into hole, protrusion and cavity, donor and acceptor etc., all implying the nature of structural and chemical match between the two interacting surfaces. In one embodiment, at least one of the heterodimers comprises an amino acid modification introduced in the immunoglobulin heavy and immunoglobulin light chains that introduce a new hydrogen bond across the light and heavy chain at the interface. In one embodiment, at least one of the heterodimers comprises an amino acid modification introduced in the immunoglobulin heavy and immunoglobulin light chains that introduces a new salt bridge across the light and heavy chain at the interface.

In one embodiment, the amino acid modifications of the Mab design set promote preferential pairing predominately through electrostatic attraction and repulsion. In one embodiment, the amino acid modifications of the Mab design set promote preferential pairing through predominantly steric mechanisms. Such Mab designs are included in Tables 4A, 4B, 7A and 7B, with selected examples including those having unique identifiers 10771-11335, 10771-11360, 10780-11417. In one embodiment the amino acid modifications of the Mab design set promote preferential pairing using both steric and electrostatic mechanisms.

In one embodiment, one or more of H1, L1, H2, and L2 comprise amino acid modifications wherein H1 and L1 do not include amino acid modifications that promote preferential pairing and H2 and L2 each comprise at least one amino acid modification that promotes preferential pairing. In one embodiment, one or more of H1, L1, H2, and L2 comprise amino acid modifications wherein one or more of H1, L1, and H2 comprise at least one amino acid modification that promotes preferential pairing, and L2 does not include amino acid modifications that promote preferential pairing. In one embodiment, one or more of H1, L1, H2, and L2 comprise amino acid modifications wherein one or more of H1, L1, and L2 comprise at least one amino acid modification that promotes preferential pairing, and H2 does not include amino acid modifications that promote preferential pairing. In one embodiment, one or more of H1, L1, H2, and L2 comprise amino acid modifications wherein one or more of H1, H2, and L2 comprise at least one amino acid modification that promotes preferential pairing and L1 does not include amino acid modifications that promote preferential pairing. In one embodiment, one or more of H1, L1, H2, and L2 comprise amino acid modifications wherein one or more of L1, H2, and L2 comprise at least one amino acid modification that promotes preferential pairing and H1 does not include amino acid modifications that promote preferential pairing. In one embodiment, one or more of H1, L1, H2, and L2 comprise amino acid modifications wherein each of L1, H2, and L2 comprise at least one amino acid modification that promotes preferential pairing.

The amino acid modifications can be in the constant domains and/or the variable domains of one or more of H1, L1, H2, and L2. In one embodiment, the amino acid modifications can be in the CH1 domains of H1 and H2, the CL-lambda domain of L1 and the CL-kappa domain of L2. In another embodiment, the amino acid modifications can be in the CH1 and VH domains of H1 and H2, the CL-lambda and VL-lambda domains of L1 and the CL-kappa and VL-kappa domains of L2. In another embodiment, the amino acid modifications can be in the VH domains of H1 and H2, the VL-lambda domain of L1 and the VL-kappa domain of L2.

In one embodiment, the amino acid modifications can be in the framework regions of one or more of H1, L1, H2 and L2. In one embodiment the amino acid modifications are limited to the conserved framework residues of the variable (VH, VL) and constant (CH1, CL) domains as indicated by the Kabat numbering of residues. For example, Almagro [Frontiers In Bioscience (2008) 13:1619-1633] provides a definition of the framework residues on the basis of Kabat, Chotia, and IMGT numbering schemes.

The number of amino acid modifications in each Mab design or Mab design set can vary. In one embodiment, H1 comprises 0 to 8 amino acid modifications, 0 to 7 amino acid modifications, 0 to 6 amino acid modifications, 0 to 5 amino acid modifications, 0 to 4 amino acid modifications, 0 to 3 amino acid modifications, 0 to 2 amino acid modifications, one amino acid modification, or no amino acid modifications. In one embodiment, L1 comprises 0 to 8 amino acid modifications, 0 to 7 amino acid modifications, 0 to 6 amino acid modifications, 0 to 5 amino acid modifications, 0 to 4 amino acid modifications, 0 to 3 amino acid modifications, 0 to 2 amino acid modifications, one amino acid modification, or no amino acid modifications. In one embodiment, H2 comprises 0 to 8 amino acid modifications, 0 to 7 amino acid modifications, 0 to 6 amino acid modifications, 0 to 5 amino acid modifications, 0 to 4 amino acid modifications, 0 to 3 amino acid modifications, 0 to 2 amino acid modifications, one amino acid modification, or no amino acid modifications. In one embodiment, L2 comprises 0 to 8 amino acid modifications, 0 to 7 amino acid modifications, 0 to 6 amino acid modifications, 0 to 5 amino acid modifications, 0 to 4 amino acid modifications, 0 to 3 amino acid modifications, 0 to 2 amino acid modifications, one amino acid modification, or no amino acid modifications.

In one embodiment, the total number of amino acid modifications in H1, L1, H2, and L2 is less than 20, less than 15, less than 12, less than 11, less than 10, less than 9, less than 8, less than 7, less than 6, less than 5, less than 4, or less than 3. In one embodiment, the total number of amino acid modifications in H1, L1, H2 and L2 is 2.

In one embodiment, the amino acid modifications can be designed specifically for a kappa-lambda system, where one parent antibody comprises a kappa light chain polypeptide sequence, and one parent antibody comprises a lambda light chain polypeptide sequence. Such amino acid modifications or designs are referred to herein as K-L designs. Examples of such amino acid modifications or K-L designs are shown in Table 4A, Table 7A, and Tables 10-A1 to 10-A12.

In another embodiment, the amino acid modifications can be initially identified with respect to a kappa-kappa system (K-K designs), where both parent antibodies comprise a kappa light chain polypeptide sequence, and subsequently ported to a kappa-lambda system. One of skill in the art would understand how these designs can be ported to a kappa-lambda system. For example, the heavy and light chains of the kappa and lambda parent antibodies can be aligned to determine the equivalent lambda light chain positions corresponding to the K-K designs. The equivalent lambda light chain positions can then be modified to conform to the K-K design. Such amino acid modifications or designs are referred to herein as K-K-derived K-L designs, and can fall into the following groups: a) those where no changes are required to the designs, and the amino acid residues modified in the kappa-kappa system are identical to those modified in the kappa-lambda system; b) those that contain silent modifications, where at least one modification made in the kappa-kappa system is unnecessary in the kappa-lambda system, because the modification naturally exists in the lambda light chain polypeptide sequence; c) those that contain amino acid modifications to at least one amino acid residue at the same relative position in the kappa light chain polypeptide sequence and the lambda light chain polypeptide sequence, but where the initial amino acid residue at that positions differs between the kappa and lambda light chain polypeptide sequences, resulting in the same amino acid modification at the position, and d) those that contain at least one additional amino acid modification in the kappa-lambda system compared to the kappa-kappa system. Examples of such K-K-derived K-L designs are provided in Table 4B, Table 7B, and Tables 10-B1 to 10-B10. Specific examples of group a) are marked by asterisk in Table 4B. A specific example of group b) is demonstrated by the Mab design set with the unique identifier 10689-10707. The silent modification is in L1 (Q160E is absent in the WT in lambda as the residue at position 160 is E and not Q). A specific example of group c) is demonstrated by the Mab design set with the unique identifier 10652-10734 where in L1, amino acid residue 124 is an E in WT lambda and a Q in the WT kappa. A specific example of group d) is demonstrated by the Mab design set with the unique identifier 10684-10706, which includes the amino acid modification K129T.

In one embodiment, one or more of H1, L1, H2, and L2 comprises amino acid modifications that promote preferential pairing of H1 with L1 as compared to L2 and of H2 with L2 as compared to L1, where the amino acid modifications comprise conservative amino acid substitutions of the Mab design sets provided in Table 4A, Table 4B, Table 7A, Table 7B, Tables 10-A1 to 10-A12, and Tables 10-B1 to 10-B10.

In one embodiment, the amino acid modifications do not introduce a new cysteine residue and do not remove a naturally occurring cysteine residue in the immunoglobulin heavy chains or the immunoglobulin light chains within the same design.

The combination of amino acid modifications in H1, L1, H2 and L2 that promote preferential pairing are referred to in general as designs. The designs may be more specifically referred to as “LCCA designs” (in the context of H1, L1, L2 or H2, L1, L2) or “Mab designs” (in the context of H1, L1, H2, L2). Typically, LCCA designs are engineered with one or more specific complementary LCCA designs based on each heavy chain of the desired bispecific antibody and accordingly are typically presented in a format where amino acid modifications in all four polypeptide chains of the bispecific antibody are identified (see for example, Tables 4A and 4B). Although specific amino acid substitutions may be identified throughout, it should be understood that conservative substitution at each amino acid position may also be contemplated. Furthermore, for the sake of illustration, the H1L1 heterodimer represents a heterodimer comprising a lambda light chain, and the H2L2 heterodimer represents a heterodimer comprising a kappa light chain, unless otherwise indicated. Finally, all amino acid residues or positions are numbered according to the Kabat numbering system, unless otherwise indicated.

The designs comprise driver sets of complementary amino acid substitutions that promote preferential pairing, and may also comprise secondary substitutions. The secondary substitutions may act to optimize the performance of the driver sets.

One or more driver sets may be employed to promote preferential pairing. These driver sets may be used individually, or in combination, to promote preferential pairing. In one embodiment, the driver set is an electrostatic driver set in which electrostatic attraction and repulsion are expected to be the predominant factors promoting preferential pairing. For example, a design in which H1 comprises the amino acid substitution 186K, L1 comprises the amino acid substitution 133D, H2 comprises the amino acid substitution 188D, and L2 comprises the amino acid substitution 131K, may promote preferential pairing by an electrostatic mechanism. Numerous other examples of electrostatic drivers are found throughout the examples. In one embodiment, one or more electrostatic driver sets may be selected from those identified in Table C:

TABLE C
Exemplary electrostatic driver sets

| Electro- | | | | |
|---|---|---|---|---|
| stastic | | | | |
| Driver | H1 | L1 | H2 | L2 |
| 1 | L143D or | T131R/K | S188K OR | S176D_T178E or S176D_T180E |
| | L143D_Q179E | | L124R_S186R | or S131D |
| 2 | L143D or | T131R/K | L143R OR S186K | Q124E_V133D or |
| | L143D_Q179E | | | Q124E_V133D_T180D |
| 3 | L143D OR | T131K/R | S186K or | V133D OR V133D_Q160E or |
| | L143D_Q179E | | L124R_S186K | Q124E_V133D or S176D_T180E |
| 4 | L143D OR | T131K/R | S188K | S131D/E |
| | L143D_Q179E | | | |
| 5 | L143D OR | T131K/R | L143R/K | Q124E_V133D or |
| | L143D_Q179E | | | Q124E_V133D_T180D |
| 6 | L143D OR | T131K/R | L124R_S186R/K OR | S176D_T178E or S176D_T180E |
| | L143D_Q179E | | L124R_Q179K OR | or S131D/E |
| | | | S188K | |
| 7 | L143D OR | T131K/R | L143R_S188K or | Q124E_V133D_S176D_T178D/E |
| | L143D_Q179E | | L143K/R or | or Q124E_V133D_T178E or |
| | | | L124R_L143K | Q124E_V133D_T180D or |
| | | | | Q124E_S176D_T180E |
| 8 | S188K | S176E/D_Y178E | V177D_S188D | S176K_T178R/K |
| | | or Y178E/D or | | |
| | | S176D_Y178T | | |
| 9 | S188K | S176E/D_Y178E | S186E or | S176K/R or S131K/R S176R/K |
| | | or Y178E/D or | L124E/L124E_Q179E | |
| | | S176D_Y178T | | |
| 10 | S186K | V133D | S188D | S131K |
| 11 | S186K | V133D | V177D_S188D | S176K_T178R/K |
| 12 | V177D_S188D | S176K_Y178K/R | S188K | S176E/D_T178E or S131D/E |
| 13 | V177D_S188D | S176K_Y178K/R | S186K/R | V133D or Q124E_Q160E_T180E |
| 14 | V177D_S188D | S176K_Y178K/R | L124R or | S176D or S176D_T178D |
| | | | L124R_Q179K or | S176D_T180E |
| | | | L124R_S186R | |
| 15 | V177D_S188D | S176K_Y178K/R | L143K/R | V133D or Q124E_V133D |
| 16 | S188E | Y178K | L124R or S188K | S176D/E_T178D/E or |
| | | | (also in combination | S176D/E_T180E or S176D |
| | | | with L143K or S186R | or in combination with Q124E or |
| | | | or Q179K) | Q124E_V133D or Q124E_Q160E |
| 17 | S186R or | S180E | L143E or | Q124R/K_T178R or |
| | Q179K | | L143E_Q179E | Q124R_Q160R/K_T178R or |
| | | | | Q124R_T129K_T178R or |
| | | | | Q124R_T129K_Q160K_T178R |
| 18 | S186K | V133D | L143E or | Q124R/K_T178R or |
| | | | L143E_Q179E | Q124R_Q160R/K_T178R or |
| | | | | Q124R_T129K_T178R or |
| | | | | Q124R_T129K_Q160K_T178R |
| 19 | L143K or | V133D or | L143E or | Q124R/K_T178R or |
| | L143K_V190K | T131E/D_V133D | L143E_Q179E | Q124R_Q160R/K_T178R or |
| | or | | | Q124R_T129K_T178R or |
| | L124K_L143K | | | Q124R_T129K_Q160K_T178R |
| 20 | S188K | S176D/E_Y178E | L143E or | Q124R/K_T178R or |
| | | or Y178E/D | L143E_Q179E | Q124R_Q160R/K_T178R or |
| | | | | Q124R_T129K_T178R or |
| | | | | Q124R_T129K_Q160K_T178R |
| 21 | Q179K or | S180E or V133D | L143E or | S131K or Q124R_T178R or |
| | S186R or | or S176E_Y178E | L143E_Q179E | Q124R_Q160K_T178R or |
| | L143K or | | | T129K_S131K |
| | S188K | | | |
| 22 | Q179K or | S180E | L143E or | S131K or Q124R_T178R or |
| | S186R | | L143E_Q179E | Q124R_Q160K_T178R |
| 23 | L143K_V190K | V133D | L124E | S131K_L135K |
| 24 | L143K or | V133D or | L124E_Q179E or | S131K/R_S176R or |
| | S186K or | T131D/E_V133D | L124E_S186E or | S131R_L135K |
| | L124K_L143K | | L124E_K143E | |
| 25 | L143E_Q179E | E124K_Y178R | S186R | T178E_T180E or Q160E_T180E |
| 26 | L143E | E124R | S186R or Q179K | Q124E_Q160E_T180E |
| 27 | S186R | S180E or | L143E and/or Q179E | Q124K_T178R or S131K |
| | | Y178E_S180E | | |
| 28 | Q179K | S180E | L143E | Q124R or |
| | | | | Q124R_Q160K_T178R |
| 29 | L124E | S176R | L124R | S176D |

In one embodiment, the driver set is a disulphide steering driver set, which may act to disfavor formation of the disulphide bond in mispaired heterodimers. An example of this type of driver set would include 125R in H1, 122D in L1, 228D in H2, and 121K in L2.

In one embodiment the driver set may be a steric driver set, which may act to promote sterically complementary interactions between correctly paired heterodimers and steric incompatibility between mispaired heterodimers. Non-limiting examples of steric driver sets are shown in Table D, where “-” indicates no amino acid substitutions are present that promote preferential pairing.

TABLE D
Exemplary steric driver sets

| Driver | H1 | L1 | H2 | L2 |
|---|---|---|---|---|
| Steric 1 | 174G | 116F_176F | 190F | 135A |
| Steric 2 | 139W | — | — | 135W |
| Steric 3 | — | — | 188W or | 176V or |
| | or | or | 186I/L_188W | 176A_178A |
| | 188A | 176A_178W | | |
| Steric 4 | — | — | 124W | 133A |
| | or | or | | |
| | 143A | 133W | | |

In one embodiment, the driver set is a variable design driver set. Such variable design driver sets comprise one or more amino acid modifications in the variable domains of the kappa and/or lambda Fabs that promote preferential pairing. In one embodiment, the variable design driver set promotes preferential pairing based on steric mechanisms. In one embodiment, the variable design drive set promotes preferential pairing based on electrostatic mechanisms. Non-limiting examples of variable design driver sets are shown in Table E, where “-” indicates no amino acid modifications present in that polypeptide that promote preferential pairing.

TABLE E
Exemplary variable domain driver sets

| Driver | H1 | L1 | H2 | L2 |
|---|---|---|---|---|
| Variable domain (steric) | 45F | — | 45A/P | 44F |
| Variable domain (steric) | — | — | 45A/P | 44F |
| Variable domain (electrostatic) | 39K/R | 38E/D | 39E/D | 38K/R |
| Variable domain (electrostatic) | 39E/D | 38K/R | 39K/R | 38E/D |
| Variable domain (electrostatic) | — | — | 39E/D | 38R/K |

In one embodiment, one or more non-naturally occurring disulphide bonds may be engineered into one or both heterodimers of the antigen-binding polypeptide construct. An example of this type of amino acid modification is one in which the heavy chain comprises a 122C substitution, paired with a 124C substitution in the kappa light chain.

Secondary substitutions may be included in a design in order to optimize the pairing performance of the design. For example, secondary substitutions may act to A) optimize the number of contacts between the heavy and the correctly paired light chain, B) provide a conducive environment for the drivers, C) optimize the hydrogen bonding network for the driver sets, or D) provide steric accommodation for the drivers. Non-limiting examples of these types of secondary substitutions are shown in Table F, where “Lk” designates a kappa light chain specific substitution, “Ll” designates a lambda light chain specific substitution. “L” designates a light chain specific substitution in either kappa or lambda light chain, and “H” designates a heavy chain specific substitution.

TABLE F
Exemplary secondary substitutions

| | | C) Hydrogen | |
|---|---|---|---|
| A) Optimizing | B) Conducive | Bonding network | D) Steric |
| contacts | environment | optimization | accommodation |
| | | | L1/178F/T |
| | | | L1/178L |
| Lk/178F | | | |
| Lk/178L | | | |
| | | | L/133A |
| | | | L/133G |
| | | | H/143I |
| | H/146G | | |
| | | | L/133I/L |
| | | | L/131S |
| | | H/143T/S | H/143T/S |
| | | Lk/178S | |
| | | H/177T | |
| H/188T | | | |
| H/177I | | | |
| | | | L/135S |
| H/188L | | | |
| H/139I | | | |
| H/190I | | | |
| | | L/133S/T | |
| | H/145T | | |
| | L1/129T | | |
| | L1/124Q | | |

Antigen-binding polypeptide constructs may be engineered with different combinations of amino acid modifications corresponding to the driver sets and secondary substitutions described above. Non-limiting specific examples of such combinations grouped into clusters based on common features are described below. As described herein, combinations of amino acid modifications at multiple positions within a single chain are identified using “_” between each position modified. For example, “124_186” indicates that both positions 124 and 186 are modified in the polypeptide chain referred to. Likewise, “124_133_180” indicates that all of positions 124, 133, and 180 are modified in the polypeptide chain referred to.

### K-L Cluster 1:

In one embodiment, the antigen-binding polypeptide construct comprises a combination of amino acid substitutions according to K-L cluster 1 wherein:
H1 comprises amino acid substitution at position 143; L1 comprises amino acid substitution at position 131; and
a) H2 comprises amino acid substitution at positions 188 or 124_186, and L2 comprises amino acid substitution at positions 176_178 or 176_180 or 131; or
b) H2 comprises amino acid substitution at position 143 or 186; and L2 comprises amino acid substitutions at positions 124_133 or 124_133_180.

In some embodiments, H1 further comprises amino acid substitutions at one or more of positions 125, 145 and 179, L1 further comprises amino acid substitution at one or more of positions 122, 124, and 133, H2 further comprises amino acid substitution at position 228, and/or L2 further comprises amino acid substitutions at position 121.

In one embodiment, the antigen-binding polypeptide construct comprises a combination of amino acid substitutions according to K-L cluster 1 wherein: H1 comprises amino acid substitution at positions 125_143_145; L1 comprises amino acid substitution at positions 122_124_131; and H2 comprises amino acid substitution at position 228, and L2 comprises amino acid substitution at positions 121_133. In some embodiments, H1 further comprises amino acid substitutions at position 179, L1 further comprises amino acid substitution at position 133, H2 further comprises amino acid substitution at one or more of positions 124, 143, 186, and 188, and L2 further comprises amino acid substitutions at one or more of positions 124, 131, 176, 178, and 180.

In some embodiments, the antigen-binding polypeptide construct comprises a combination of amino acid substitutions according to K-L cluster 1 wherein H1 comprises amino acid substitutions at positions 125_143_145, or 125_143_145_179; L1 comprises amino acid substitutions at positions 122_124_131, or 122_124_131_133; H2 comprises amino acid substitutions at positions 124_186_228, 143_228, 143_186_228, 186_228, or 188_228, and L2 comprises amino acid substitutions at positions 121_124_133, 121_124_133_180, 121_131_133_178, 121_133_176_178, or 121_133_176_180.

In some embodiments, the antigen-binding polypeptide construct comprises a combination of amino acid substitutions according to K-L cluster 1 wherein the amino acid substitutions in H1 are selected from 125R, 145T, 143D, 143E, 179E, and conservative substitutions thereof; the amino acid substitutions in L1 are selected from 124Q, 122D, 131K, 131R, 133S, and conservative substitutions thereof; the amino acid substitutions in H2 are selected from 228D, 124R, 143I, 143R, 186K, 186R, 188K, and conservative substitutions thereof; the amino acid substitutions in L2 are selected from 124E, 121K, 131D, 176D, 178E, 178F, 180D, 180E, 133D, 133G, 133I, and conservative substitutions thereof.

In some embodiments, the antigen-binding polypeptide construct has an H1L1 heterodimer comprising a combination of amino acid substitutions according to K-L cluster 1, wherein the H1L1 heterodimer comprises one of the following sets of amino acid substitutions:

| H1 | L1 |
|---|---|
| 125R_143D_145T | 122D_124Q_131K_133S |
| 125R_143E_145T_179E | 122D_124Q_131R |
| 125R_143E_145T_179E | 122D_124Q_131K |

In one embodiment, H1 comprises 125R_143E_145T_179E and L1 comprises 122D_124Q_131R. In another embodiment, H1 comprises 125R_143E_145T and L1 comprises 122D_124Q_131R. In further embodiments, the antigen-binding polypeptide construct has an H2L2 heterodimer comprising a combination of amino acid substitutions according to K-L cluster 1, wherein the H2L2 heterodimer comprises one of the following sets of amino acid substitutions:

| H2 | L2 |
|---|---|
| 188K_228D | 121K_133I_176D_178E |
| 188K_228D | 121K_131D_133G_178F |
| 124R_186R_228D | 121K_133G_176D_180E |
| 124R_186R_228D | 121K_133G_176D_180E |
| 143R_228D | 121K_124E_133D_180D |
| 143R_228D | 121K_124E_133D |
| 143I_186K_228D | 121K_124E_133D |
| 186K_228D | 121K_124E_133D |

In some embodiments, the antigen-binding polypeptide construct comprises a combination of one H1L1 heterodimer with one H2L2 heterodimer. In one embodiment, H1 comprises 125R_143E_145T_179E, L1 comprises 122D_124Q_131R, H2 comprises 188K_228D, and L2 comprises 121K_133I_176D_178E. In another embodiment, H1 comprises 125R_143D_145T, L1 comprises 122D_124Q_131R, H2 comprises 143R_228D, and L2 comprises 121K_124E_133D.

In some embodiments, the antigen-binding polypeptide construct comprises a combination of amino acid substitutions according to K-L cluster 1, comprising a disulfide steering driver set.

In one embodiment, the amino acid combinations of K-L cluster 1 comprises one or more secondary substitutions selected from Table F.

In one embodiment, the antigen-binding polypeptide construct comprises a combination of amino acid substitutions according to K-L cluster 1 as set forth in one or more of the designs in Table 10-A1.

### K-L Cluster 2:

In one embodiment, the antigen-binding polypeptide construct comprises a combination of amino acid substitutions according to K-L cluster 2 wherein:
H1 comprises amino acid substitution at position 143; L1 comprises amino acid substitution at position 131; and
c) H2 comprises amino acid substitution at positions 186 or 124_186, and L2 comprises amino acid substitution at positions 133 or 133_160 or 124_133 or 176_180; or
d) H2 comprises amino acid substitution at position 188; and L2 comprises amino acid substitutions at position 131;
e) H2 comprises amino acid substitution at position 143; and L2 comprises amino acid substitutions at positions 124_133 or 124_133_180;

In some embodiments, H1 further comprises amino acid substitutions at one or more of positions 139, 145, 174, and 179, L1 further comprises amino acid substitutions at one or more of positions 116, 124, 133 and 176, H2 further comprises amino acid substitution at one or more of positions 190, 39, and 45, and/or L2 further comprises amino acid substitutions at one or more of positions 135, 178, 38, and 44.

In one embodiment, the antigen-binding polypeptide construct comprises a combination of amino acid substitutions according to K-L cluster 2 wherein H1 comprises amino acid substitution at positions 143_145; L1 comprises amino acid substitution at position 131; and H2 comprises amino acid substitutions at position 143 or 186 or 188, and L2 comprises amino acid substitution at position 133. In some embodiments, H1 further comprises amino acid substitutions at one or more of positions 139, 174, and 179, L1 further comprises amino acid substitution at one or more of positions 116, 124, 133, and 176, H2 further comprises amino acid substitution at one or more of positions, 124, 190, 39, and 45 and L2 further comprises amino acid substitutions at one or more of positions 124, 131, 135, 160, 176, 178, 180, 38, 44.

In some embodiments, the antigen-binding polypeptide construct comprises a combination of amino acid substitutions according to K-L cluster 2 wherein H1 comprises amino acid substitutions at positions 139_143_145, 143_145, 143_145_174, or 143_145_179; L1 comprises amino acid substitutions at positions 116_124_131_176, 124_131, 124_131_133, 124_131_133_176, 131, or 131_133; H2 comprises amino acid substitutions at positions 124_186_190, 143, 143_186, 143_186_190, 143_190, 186, 186_190, 188, 39_143, or 45_143, and L2 comprises amino acid substitutions at positions 124_133, 124_133_135, 124_133_135_180, 124_133_180, 131_133_135_178, 131_133_178, 133, 133_135_176_180, 133_160, 38_124_133, or 44_124_133.

In some embodiments, the antigen-binding polypeptide construct comprises a combination of amino acid substitutions according to K-L cluster 2 wherein the amino acid substitutions in H1 are selected from 139W, 143D, 145T, 174G, 179E, and conservative substitutions thereof; the amino acid substitutions in L1 are selected from 124Q, 176F, 116F, 131K, 131R, 133S, and conservative substitutions thereof; the amino acid substitutions in H2 are selected from 124R, 143I, 143K, 143R, 186K, 188K, 190F, 39E, 45P, and conservative substitutions thereof; the amino acid substitutions in L2 are selected from 124E, 131D, 131E, 133D, 133G, 135A, 135W, 160E, 176D, 178F, 180D, 180E, 38R, 44F, and conservative substitutions thereof.

In some embodiments, the antigen-binding polypeptide construct has an H1L1 heterodimer comprising a combination of amino acid substitutions according to K-L cluster 2, wherein the H1L1 heterodimer comprises one of the following sets of amino acid substitutions:

| H1 | L1 |
|---|---|
| 139W_143D_145T | 124Q_131K_133S |
| 139W_143D_145T | 124Q_131R |
| 143D_145T | 124Q_131K |
| 143D_145T | 124Q_131K_133S |
| 143D_145T | 124Q_131R |
| 143D_145T | 131K |
| 143D_145T | 131K_133S |
| 143D_145T_174G | 116F_124Q_131R_176F |
| 143D_145T_174G | 124Q_131K_133S_176F |
| 143D_145T_174G | 116F_124Q_131K_176F |
| 143D_145T_179E | 124Q_131K |
| 143D_145T_179E | 124Q_131K_133S |
| 143D_145T_179E | 131K |
| 143D_145T_179E | 131K_133S |

In further embodiments, the antigen-binding polypeptide construct has an H2L2 heterodimer comprising a combination of amino acid substitutions according to K-L cluster 2, wherein the H2L2 heterodimer comprises one of the following sets of amino acid substitutions:

| H2 | L2 |
|---|---|
| 143I_186K | 133D |
| 143I_186K | 133D_160E |
| 143I_186K | 124E_133D_135W |
| 143I_186K_190F | 124E_133D_135A |
| 186K | 124E_133D |
| 186K | 133D_160E |
| 186K | 133D |
| 186K | 124E_133D_135W |
| 186K_190F | 124E_133D_135A |
| 124R_186K_190F | 133G_135A_176D_180E |
| 188K | 131D_133G_135W_178F |
| 188K | 131D_133G_178F |
| 188K | 131E_133G_178F |
| 143K/R | 124E_133D |
| 143R | 124E_133D_135W_180D |
| 143R | 124E_133D_135W |
| 143R | 124E_133D_180D |
| 143R_190F | 124E_133D_135A |
| 45P_143R | 44F_124E_133D |
| 39E_143R | 38R_124E_133D |

In some embodiments, the antigen-binding polypeptide construct comprises a combination of one H1L1 heterodimer with one H2L2 heterodimer. In one embodiment, the combination of H1L1 heterodimer and H2L2 heterodimer is one of the following:

| H1 | L1 | H2 | L2 |
|---|---|---|---|
| 143D_145T | 124Q_131R | 188K | 131D_133G_178F |
| 143D_145T_174G | 116F_124Q_131R_176F | 143R_190F | 124E_133D_135A |
| 139W_143D_145T | 124Q_131R | 143R | 124E_133D_135W |
| 143D_145T | 124Q_131R | 143R | 124E_133D_180D |

In some embodiments, the antigen-binding polypeptide construct comprises a combination of amino acid substitutions according to K-L cluster 2, having one or more of steric 1, steric 2, variable domain steric, or variable domain electrostatic driver sets.

In one embodiment, the amino acid combinations of K-L cluster 2 comprises one or more secondary substitutions selected from Table F.

In one embodiment, the antigen-binding polypeptide construct comprises a combination of amino acid substitutions according to K-L cluster 2 as set forth in one or more of the designs in Table 10-A2.

### K-L Cluster 3:

In one embodiment, the antigen-binding polypeptide construct comprises a combination of amino acid substitutions according to K-L cluster 3 wherein:
H1 comprises amino acid substitution at position 143; L1 comprises amino acid substitution at position 131; and
a) H2 comprises amino acid substitution at positions 124_186 or 124_179 or 188, and L2 comprises amino acid substitution at positions 176_178 or 176_180 or 131; or
b) H2 comprises amino acid substitution at positions 143_188 or 143 or 124_143; and L2 comprises amino acid substitutions at positions 124_176_178 or 124_178 or 124_180 or 124_176_180, or 124, or 124_176.

In some embodiments, H1 further comprises amino acid substitutions at one or more of positions 139, 145, 174, and 179, L1 further comprises amino acid substitution at one or more of positions 116, 124, and 176, H2 further comprises amino acid substitution at one or more of positions 177, 190, 39, and 45, and/or L2 further comprises amino acid substitutions at one or more of positions 133, 135, 178, 38, and 44.

In one embodiment, the antigen-binding polypeptide construct comprises a combination of amino acid substitutions according to K-L cluster 3 wherein: H1 comprises amino acid substitution at positions 143_145; L1 comprises amino acid substitution at positions 124_131; and H2 comprises amino acid substitution at one or more of positions 143, 124, and 188, and L2 comprises amino acid substitution at positions 133 or 176_178. In some embodiments, H1 further comprises amino acid substitutions at one or more of positions 139, 174, and 179, L1 further comprises amino acid substitution at position 116 or 176, H2 further comprises amino acid substitution at one or more of positions 177, 179, 186, 190, 39, and 45, and/or L2 further comprises amino acid substitutions at one or more of positions 124, 131, 135, 180, 38, and 44.

In some embodiments, the antigen-binding polypeptide construct comprises a combination of amino acid substitutions according to K-L cluster 3 wherein H1 comprises amino acid substitutions at positions 139_143_145, 139_143_145_179, 143_145, 143_145_174_179, or 143_145_179; L1 comprises amino acid substitutions at positions 116_124_131_176, or at 124_131; H2 comprises amino acid substitutions at positions 124_143, 124_179, 124_186, 143, 143_188, 177_188, 188, 188_190, 39_124_179, or 45_124_179, and L2 comprises amino acid substitutions at positions 124_133, 124_133_176, 124_133_176_178, 124_133_176_180, 124_133_178, 124_133_180, 131_133_178, 133_135_176_178, 133_135_176_180, 133_176_178, 133_176_180, 176_178, 38_133_176_180, or 44_133_176_180.

In some embodiments, the antigen-binding polypeptide construct comprises a combination of amino acid substitutions according to K-L cluster 3 wherein the amino acid substitutions in H1 are selected from 139W, 174G, 145T, 143D, 143E, 179E, and conservative substitutions thereof; the amino acid substitutions in L1 are selected from 116F, 124Q, 131K, 131R, 176F, and conservative substitutions thereof; the amino acid substitutions in H2 are selected from 124R, 143K, 143R, 177I, 179K, 186K, 186R, 188K, 190F, 39E, 45P, and conservative substitutions thereof; the amino acid substitutions in L2 are selected from 135A, 135W, 44F, 124E, 38R, 131D, 131E, 176D, 176E, 178D, 178E, 178F, 180D, 180E, 133D, 133G, 133I, 133L, and conservative substitutions thereof.

In some embodiments, the antigen-binding polypeptide construct has an H1L1 heterodimer comprising a combination of amino acid substitutions according to K-L cluster 3, wherein the HILL heterodimer comprises one of the following sets of amino acid substitutions:

| H1 | L1 |
|---|---|
| 139W_143D_145T | 124Q_131R |
| 139W_143E_145T_179E | 124Q_131K |
| 143D_145T | 124Q_131R |
| 143E_145T_179E | 124Q_131R |
| 143E_145T_174G_179E | 116F_124Q_131R_176F |

In further embodiments, the antigen-binding polypeptide construct has an H2L2 heterodimer comprising a combination of amino acid substitutions according to K-L cluster 3, wherein the H2L2 heterodimer comprises one of the following sets of amino acid substitutions:

| H2 | L2 |
|---|---|
| 124R_179K | 133G_176D_178E |
| 124R_186K/R | 133G_176D_180E |
| 124R_186K/R | 133G_135W_176D_180E |
| 188K | 131D_133G_178F |
| 188K | 131E_133G_178F |
| 188K | 176E_178E |
| 188K | 133I_135W_176D_178E |
| 188K | 133I_176D_178E |
| 143R_188K | 124E_133D_176D_178D |
| 143R_188K | 124E_133D_176D_178E |
| 143R_188K | 124E_133D_178E |
| 143K/R | 124E_133D |
| 143K/R | 124E_133D_180D |
| 124R_143K | 124E_133G_176D_180E |
| 124R_143K | 124E_133G_176D |

In some embodiments, the antigen-binding polypeptide construct comprises a combination of one H1L1 heterodimer with one H2L2 heterodimer. In one embodiment, the combination of H1L1 heterodimer and H2L2 heterodimer is one of the following:

| H1 | L1 | H2 | L2 |
|---|---|---|---|
| 143E_145T_179E | 124Q_131K | 124R_186K | 133G_176D_178E |
| 143E_145T_179E | 124Q_131K | 143K | 124E_133D |
| 143D_145T | 124Q_131R | 188K | 176E_178E |
| 143D_145T | 124Q_131K | 124R_186R | 133G_176D_180E |
| 139W_143D_145T | 124Q_131R | 124R_186K | 133G_135W_176D_180E |

In some embodiments, the antigen-binding polypeptide construct comprises a combination of amino acid substitutions according to K-L cluster 3, having one or more of steric 1, steric 2, variable domain steric or variable domain electrostatic driver sets.

In one embodiment, the amino acid combinations of K-L cluster 3 comprise one or more secondary substitutions selected from Table F.

In one embodiment, the antigen-binding polypeptide construct comprises a combination of amino acid substitutions according to K-L cluster 3, as set forth in one or more of the designs in Table 10-A3.

### K-L Cluster 4:

In one embodiment, the antigen-binding polypeptide construct comprises a combination of amino acid substitutions according to K-L cluster 4 wherein: H1 comprises amino acid substitution at position 188; L1 comprises amino acid substitution at positions 176_178 or 178; and
a) H2 comprises amino acid substitution at positions 177_188, and L2 comprises amino acid substitution at positions 176_178; or
b) H2 comprises amino acid substitution at position 186 or 124 or 124_179; and L2 comprises amino acid substitutions at positions 176 or 131_176.

In some embodiments, H1 further comprises amino acid substitutions at one or more of positions 125, 139, and 177, L1 further comprises amino acid substitution at one or more of positions 122, 129, and 133, H2 further comprises amino acid substitution at one or more of positions 145, 228, 45, and 39, and/or L2 further comprises amino acid substitutions at one or more of positions 135, 44, 38, 121, and 133.

In one embodiment, the antigen-binding polypeptide construct comprises a combination of amino acid substitutions according to K-L cluster 4 wherein: H1 comprises no amino acid substitutions that promote preferential pairing or comprises amino acid substitution at position 188; L1 comprises no amino acid substitutions that promote preferential pairing or comprises amino acid substitution at positions 176_178; H2 comprises amino acid substitution at positions 188 or 186_188, and L2 comprises amino acid substitution at positions 176_178.

In one embodiment, the antigen-binding polypeptide construct comprises a combination of amino acid substitutions according to K-L cluster 4 wherein: H1 comprises no amino acid substitutions that promote preferential pairing or comprises amino acid substitution at position 188; L1 comprises no amino acid substitutions that promote preferential pairing or comprises amino acid substitution at positions 178; H2 comprises amino acid substitution at one or more of positions 124, 186, and 188, and L2 comprises amino acid substitution at position 176. In some embodiments, H1 further comprises amino acid substitutions at one or more of positions 125, 139, and 177, L1 further comprises amino acid substitution at one or more of positions 122, 129, 133, and 176, H2 further comprises amino acid substitution at one or more of positions 145, 228, 45, 177, 179, and 39, and/or L2 further comprises amino acid substitutions at one or more of positions 135, 44, 38, 121, 131, 178, and 133.

In some embodiments, the antigen-binding polypeptide construct comprises a combination of amino acid substitutions according to K-L cluster 4 wherein H1 comprises no amino acid substitutions that promote preferential pairing or comprises amino acid substitutions at positions 125_188, 139_188, 188, or 177_188; L1 comprises no amino acid substitutions that promote preferential pairing or comprises amino acid substitutions at positions 129_176_178, 129_178, 122_129_176_178, 176_178, or 133_176_178; H2 comprises amino acid substitutions at positions 145_186, 145_186_228, 145_177_188, 124, 124_145_179, 124_145_179_186_188, 124_145_179_188, 124_186_188, 124_188, 45_124_145_179, 39_124_145_179, or 186_188, and L2 comprises amino acid substitutions at positions 44_131_133_176, 38_131_133_176, 121_131_176, 131_135_176, 131_176, 131_133_176, 131_133_176_178, 176, 176_178, 133_176, or 133_176_178.

In some embodiments, the antigen-binding polypeptide construct comprises a combination of amino acid substitutions according to K-L cluster 4 wherein the amino acid substitutions in H1 are selected from 125R, 139W, 188A, 188K, and 177I, and conservative substitutions thereof; the amino acid substitutions in L1 are selected from 129T, 122D, 176A, 176D, 176E, 133I, 133L, 178D, 178E, 178T, and 178W, and conservative substitutions thereof; the amino acid substitutions in H2 are selected from 124E, 145T, 177D, 179E, 186E, 186I, 186L, 188D, 188W, 228D, 39E, and 45P, and conservative substitutions thereof; the amino acid substitutions in L2 are selected from 121K, 131K, 131R, 133A, 133G, 135W, 176A, 176K, 176R, 176V, 178A, 178K, 178L, 178R, 38R, and 44F, and conservative substitutions thereof.

In some embodiments, the antigen-binding polypeptide construct has an H1L1 heterodimer comprising a combination of amino acid substitutions according to K-L cluster 4, wherein the H1L1 heterodimer comprises one of the following sets of amino acid substitutions:

In further embodiments, the antigen-binding polypeptide construct has an H2L2 heterodimer comprising a combination of amino acid substitutions according to K-L cluster 4, wherein the H2L2 heterodimer comprises one of the following sets of amino acid substitutions:

| H2 | L2 |
|---|---|
| 145T_186E | 131K_135W_176K |
| 145T 186E_228D | 131K_176K |
| 145T_186E_228D | 121K_131K_176K |
| 124E | 133G_176R |
| 124E | 133A_176K |
| 124E_186I_188W | 133G_176R_178A |
| 124E_188W | 133A_176K_178A |
| 124E_145T_179E | 131K_133G_176R |
| 124E_145T_179E | 131R_133G_176R |
| 124E_145T_179E_186I_188W | 131R_133G_176R_178A |
| 124E_145T_179E_188W | 131K_133G_176R_178A |
| 45P_L124E_145T_179E | 44F_131R_133G_176R |
| 39E_L124E_145T_179E | 38R_131R_133G_176R |

In some embodiments, the antigen-binding polypeptide construct comprises a combination of one H1L1 heterodimer with one H2L2 heterodimer. In one embodiment, the combination of H1L1 heterodimer and H2L2 heterodimer is one of the following:

| H1 | L1 | H2 | L2 |
|---|---|---|---|
| 188K | 176E_178E | 124E_145T_179E | 131R_133G_176R |
| 125R_188K | 122D_129T_176E_178E | 145T_186E_228D | 121K_131K_176K |
| 188K | 129T_178D | 145T_186E | 131K_176K |

In some embodiments, the antigen-binding polypeptide construct comprises a combination of amino acid substitutions according to K-L cluster 4, having one or more of electrostatic, disulfide steering, steric 3, variable domain steric and variable domain electrostatic driver sets.

In one embodiment, the amino acid combinations of K-L cluster 4 comprise one or more secondary substitutions selected from Table F.

In one embodiment, the antigen-binding polypeptide construct comprises a combination of amino acid substitutions according to K-L cluster 4 as set forth in one or more of the designs in Table 10-A4.

### K-L Cluster 5:

In one embodiment, the antigen-binding polypeptide construct comprises a combination of amino acid substitutions according to K-L cluster 5 wherein:
H1 comprises amino acid substitution at position 186; L1 comprises amino acid substitution at position 133; and
a) H2 comprises amino acid substitution at position 188, and L2 comprises amino acid substitution at position 131; or
b) H2 comprises amino acid substitution at positions 177_188; and L2 comprises amino acid substitutions at positions 176_178; or
wherein H1 comprises amino acid substitution at positions 124_190; L1 comprises amino acid substitution at position 135; H2 comprises amino acid substitution at positions 124 or 188, and L2 comprises amino acid substitution at positions 176 or 176_178.

In some embodiments, H1 further comprises amino acid substitutions at position, 143 and/or 188, L1 further comprises amino acid substitution at position 131 and/or 178, H2 further comprises amino acid substitution at positions 143 and/or 145, and/or L2 further comprises amino acid substitutions at position 133 and/or 178.

In one embodiment, the antigen-binding polypeptide construct comprises a combination of amino acid substitutions according to K-L cluster 5 wherein: H1 comprises amino acid substitution at position 186 or 124; L1 comprises amino acid substitution at position 133 and/or 135; and H2 comprises amino acid substitutions at one or more of positions 188, 177 and 124, and L2 comprises amino acid substitution at position 176 and/or 131.

In some embodiments, H1 further comprises amino acid substitutions at one or more of positions 143, 188, and 190, L1 further comprises amino acid substitution at positions 131 and/or 178, H2 further comprises amino acid substitution at positions 143 and/or 145, and/or L2 further comprises amino acid substitutions at positions 133 and/or 178.

In some embodiments, the antigen-binding polypeptide construct comprises a combination of amino acid substitutions according to K-L cluster 5 wherein H1 comprises amino acid substitutions at positions 124_190, 143_186_188, or 186_188; L1 comprises amino acid substitutions at positions 131_133_178, 133_135, 133_135_178, 133_178, or 135_178; H2 comprises amino acid substitutions at positions 124, 143_188, 145_177_188, or 177_188, and L2 comprises amino acid substitutions at positions 131_176_178, 131_178, 133_176, 133_176_178, or 176_178.

In some embodiments, the antigen-binding polypeptide construct comprises a combination of amino acid substitutions according to K-L cluster 5 wherein the amino acid substitutions in H1 are selected from 124E, 143S, 186K, 188T, 190D, 190E, and conservative substitutions thereof; the amino acid substitutions in L1 are selected from 131S, 133D, 133I, 135K, 135R, 178F, 178T, and conservative substitutions thereof; the amino acid substitutions in H2 are selected from 124R, 143T, 145T, 177D, 177I, 188D, 188K, and conservative substitutions thereof; the amino acid substitutions in L2 are selected from 131K, 133G, 133L, 176A, 176D, 176K, 178E, 178K, 178R, 178S, and conservative substitutions thereof.

In some embodiments, the antigen-binding polypeptide construct has an H1L1 heterodimer comprising a combination of amino acid substitutions according to K-L cluster 5, wherein the H1L1 heterodimer comprises one of the following sets of amino acid substitutions:

| H1 | L1 |
|---|---|
| 186K_188T | 133D_178T |
| 186K_188T | 131S_133D_178T |
| 143S_186K_188T | 133D_178T |
| 124E_190E/D | 133I_135R_178F |
| 124E_190E/D | 133I_135K_178F |
| 124E_190E/D | 133I_135K |
| 124E_190E/D | 135K_178F |

In further embodiments, the antigen-binding polypeptide construct has an H2L2 heterodimer comprising a combination of amino acid substitutions according to K-L cluster 5, wherein the H2L2 heterodimer comprises one of the following sets of amino acid substitutions:

| H2 | L2 |
|---|---|
| 145T_177D_188D | 176K_178K |
| 145T_177D_188D | 176K_178R |
| 124R | 133G_176D |
| 143T_188D | 131K_178S |
| 143T_188D | 131K_176A_178S |
| 177I_188K | 133L_176D_178E |

In some embodiments, the antigen-binding polypeptide construct comprises a combination of one H1L1 heterodimer with one H2L2 heterodimer. In one embodiment, the combination of H1L1 heterodimer and H2L2 heterodimer is one where H1 comprises 186K_188T, L1 comprises 133D_178T, H2 comprises 145T_177D_188D, and L2 comprises 176K_178K.

In one embodiment, the amino acid combinations of K-L cluster 5 comprise one or more secondary substitutions selected from Table F.

In one embodiment, the antigen-binding polypeptide construct comprises a combination of amino acid substitutions according to K-L cluster 5, as set forth in one or more of the designs in Table 10-A5.

### K-L Cluster 6:

In one embodiment, the antigen-binding polypeptide construct comprises a combination of amino acid substitutions according to K-L cluster 6 wherein:
H1 comprises amino acid substitution at positions 177_188; L1 comprises amino acid substitution at position 176_178; and
a) H2 comprises amino acid substitution at position 188, and L2 comprises amino acid substitution at positions 176_178 or 131;
b) H2 comprises amino acid substitution at position 186; and L2 comprises amino acid substitutions at positions 133 or 124_160_180;
c) H2 comprises amino acid substitution at position 124 or 124_179 or 124_186; and L2 comprises amino acid substitutions at positions 176 or 176_178 or 176_180; or
d) H2 comprises amino acid substitution at position 143; and L2 comprises amino acid substitutions at positions 133 or 124_133.

In some embodiments, H1 further comprises amino acid substitutions at 145 and/or 146, and/or H2 further comprises amino acid substitution at one or more of positions 143 and/or 177.

In one embodiment, the antigen-binding polypeptide construct comprises a combination of amino acid substitutions according to K-L cluster 6 wherein:
H1 comprises amino acid substitution at positions 177_188; L1 comprises amino acid substitution at positions 176_178; and H2 comprises amino acid substitution at one or more of positions 124, 143, 179, 186, and 188, and L2 comprises amino acid substitution at one or more of positions 133, 176, and 178.

In some embodiments, H1 further comprises amino acid substitutions at 145 and/or 146 positions, H2 further comprises amino acid substitution at position 177, and/or L2 further comprises amino acid substitutions at one or more of positions 124, 131, 160, and 180.

In some embodiments, the antigen-binding polypeptide construct comprises a combination of amino acid substitutions according to K-L cluster 6 wherein H1 comprises amino acid substitutions at positions 145_177_188, or 146_177_188; L1 comprises amino acid substitutions at positions 176_178; H2 comprises amino acid substitutions at positions 124, 124_179, 124_186, 143, 143_186_188, 177_188, 179, 186, 186_188, or 188, and L2 comprises amino acid substitutions at positions 124_133, 124_160_176_178_180, 124_160_180, 131_133_178, 133, 133_176, 133_176_178, 133_176_180, or 176_178.

In some embodiments, the antigen-binding polypeptide construct comprises a combination of amino acid substitutions according to K-L cluster 6 wherein the amino acid substitutions in H1 are selected from 145T, 146T, 177D, 188D, and conservative substitutions thereof; the amino acid substitutions in L1 are selected from 176K, 178K, 178L, 178R, and conservative substitutions thereof; the amino acid substitutions in H2 are selected from 124R, 143K, 143R, 143S, 177I, 179K, 186K, 186R, 188K, 188T, 188W, and conservative substitutions thereof; the amino acid substitutions in L2 are selected from 124E, 131D, 131E, 133D, 133G, 133I, 133L, 160E, 176A, 176D, 176E, 178A, 178D, 178E, 178F, 180E, and conservative substitutions thereof.

In some embodiments, the antigen-binding polypeptide construct has an H1L1 heterodimer comprising a combination of amino acid substitutions according to K-L cluster 6, wherein the HILL heterodimer comprises one of the following sets of amino acid substitutions:

| H1 | L1 |
|---|---|
| 146T_177D_188D | 176K_178K |
| 145T_177D_188D | 176K_178L |
| 145T_177D_188D | 176K_178R |

In further embodiments, the antigen-binding polypeptide construct has an H2L2 heterodimer comprising a combination of amino acid substitutions according to K-L cluster 6, wherein the H2L2 heterodimer comprises one of the following sets of amino acid substitutions:

| H2 | L2 |
|---|---|
| 124R | 133G_176D |
| 124R | 133G_176D_178D |
| 124R_179K | 133G_176D_180E |
| 124R_186R | 133G_176D_180E |
| 143K | 124E_133D |
| 143K | 133D |
| 179K or 186R | 124E_160E_180E |
| 186R_188W | 124E_160E_176A_178A_180E |
| 1438_186K_188T | 133D |
| 186K_188T | 133D |
| 188K | 131D_133G_178F |
| 188K | 131E_133G_178F |
| 188K | 176D_178E |
| 188K | 176E_178E |
| 177I_188K | 133I_176E_178E |
| 177I_188K | 133L_176D_178E |

In some embodiments, the antigen-binding polypeptide construct comprises a combination of one H1L1 heterodimer with one H2L2 heterodimer. In one embodiment, the combination of H1L1 heterodimer and H2L2 heterodimer is one of the following:

| H1 | L1 | H2 | L2 |
|---|---|---|---|
| 145T_177D_188D | 176K_178K | 124R | 133G_176D |
| 145T_177D_188D | 176K_178K | 143K | 124E_133D |

In some embodiments, the antigen-binding polypeptide construct comprises a combination of amino acid substitutions according to K-L cluster 6, having a steric driver set.

In one embodiment, the amino acid combinations of K-L cluster 6 comprise one or more secondary substitutions selected from Table F.

In one embodiment, the antigen-binding polypeptide construct comprises a combination of amino acid substitutions according to K-L cluster 6, as set forth one or more of the designs in Table 10-A6.

### K-L Cluster 7:

In one embodiment, the antigen-binding polypeptide construct comprises a combination of amino acid substitutions according to K-L cluster 7 wherein:
H1 comprises amino acid substitution at position 188; L1 comprises amino acid substitution at position 178; H2 comprises amino acid substitution at position 124 or 188, and L2 comprises amino acid substitution at positions 176_178 or 176_180 or 176. In some embodiments, H1 further comprises amino acid substitutions at one or more of positions 125, 139, 145, and 177, L1 further comprises amino acid substitution at position 122, H2 further comprises amino acid substitution at one or more of positions 143, 177, 179, 186, 228, 39, and 45, and/or L2 further comprises amino acid substitutions at one or more of positions 121, 124, 133, 135, 160, 38, and 44.

In one embodiment, the antigen-binding polypeptide construct comprises a combination of amino acid substitutions according to K-L cluster 7 wherein: H1 comprises amino acid substitution at positions 145_188; L1 comprises amino acid substitution at position 178; and H2 comprises amino acid substitution at position 124 and/or 188, and L2 comprises amino acid substitution at one or more of positions 124, 133, and 178.

In some embodiments, H1 further comprises amino acid substitutions at one or more of positions 125, 139, and 177, L1 further comprises amino acid substitution at position 122, H2 further comprises amino acid substitution at one or more of positions 143, 177, 179, 186, 228, 39, and 45, and/or L2 further comprises amino acid substitutions at one or more of positions 121, 135, 160, 176, 180, 38, and 44.

In some embodiments, the antigen-binding polypeptide construct comprises a combination of amino acid substitutions according to K-L cluster 7 wherein H1 comprises amino acid substitutions at positions 125_145_188, 139_145_188, 145_177_188, or 145_188; L1 comprises amino acid substitutions at positions 122_178, or 178; H2 comprises amino acid substitutions at positions 124, 124_143, 124_179, 124_186, 124_186_228, 124_188, 124_228, 143_188, 177_188, 179_188, 186_188, 188, 188_228, 39_124_179, or 45_124_179, and L2 comprises amino acid substitutions at positions 121_133_176, 121_133_176_180, 121_176_178, 124_133_176, 124_133_176_178, 124_133_176_178_180, 124_133_176_180, 124_133_178, 124_160_176_178, 124_160_176_178_180, 124_176_178_180, 124_176_180, 133_135_176, 133_135_176_180, 133_176, 133_176_178, 133_176_180, 135_176_178, 176_178, 38 133_176_180, or 44_133_176_180.

In some embodiments, the antigen-binding polypeptide construct comprises a combination of amino acid substitutions according to K-L cluster 7 wherein the amino acid substitutions in H1 are selected from 125R, 139W, 145T, 177T, 188E, and conservative substitutions thereof; the amino acid substitutions in L1 are selected from 122D, 178K, and conservative substitutions thereof; the amino acid substitutions in H2 are selected from 124K, 124R, 143K, 143R, 177I, 179K, 186R, 188K, 228D, 39E, 45P, and conservative substitutions thereof; the amino acid substitutions in L2 are selected from 121K, 124E, 133D, 133G, 133L, 135W, 160E, 176D, 176E, 178D, 178E, 180E, 38R, 44F, and conservative substitutions thereof.

In some embodiments, the antigen-binding polypeptide construct has an H1L1 heterodimer comprising a combination of amino acid substitutions according to K-L cluster 7, wherein the H1L1 heterodimer comprises one of the following sets of amino acid substitutions:

| H1 | L1 |
|---|---|
| 125R_145T_188E | 122D_178K |
| 139W_145T_188E | 178K |
| 145T_188E | 178K |
| 145T_177T_188E | 178K |

In further embodiments, the antigen-binding polypeptide construct has an H2L2 heterodimer comprising a combination of amino acid substitutions according to K-L cluster 7, wherein the H2L2 heterodimer comprises one of the following sets of amino acid substitutions:

| H2 | L2 |
|---|---|
| 124R | 133G_176D |
| 124R | 133G_135W_176D |
| 124R | 133G_135W_176D_180E |
| 124R | 133G_176D_180E |
| 124R_228D | 121K_133G_176D |
| 124R_186R_228D | 121K_133G_176D_180E |
| 188K_228D | 121K_176E_178E |
| 124R_143K | 124E_133G_176D_180E |
| 124R_143K | 124E_133G_176D |
| 124R_143K | 124E_133G_176D_178E_180E |
| 124R_179K | 133G_176D_180E |
| 124R_186R | 133G_135W_176D_180E |
| 45P_124R_179K | 44F_133G_176D_180E |
| 39E_124R_179K | 38R_133G_176D_180E |
| 124K_188K | 124E_133G_176D_178D |
| 124R_188K | 124E_133D_176E_178E_180E |
| 143K_188K | 124E_133D_176E_180E |
| 143K_188K | 124E_133D_176D_178E |
| 143K_188K | 124E_133D_178E |
| 179K_188K | 124E_176E_180E |
| 179K_188K | 124E_176E_178D_180E |
| 179K_188K | 124E_176E_178E_180E |
| 186R_188K | 124E_160E_176D_178D_180E |
| 186R_188K | 124E_160E_176D_178D |
| 188K | 135W_176E_178E |
| 188K | 176D_178E |
| 188K | 176E_178E |
| 177I_188K | 133L_176D_T178E |

In some embodiments, the antigen-binding polypeptide construct comprises a combination of one H1L1 heterodimer with one H2L2 heterodimer. In one embodiment, the combination of H1L1 heterodimer and H2L2 heterodimer is one of the following:

| H1 | L1 | H2 | L2 |
|---|---|---|---|
| 145T_188E | 178K | 143R_188K | 124E_133D_178E |
| 125R_145T_188E | 122D_178K | 188K_228D | 121K_176E_178E |
| 145T_188E | 178K | 124R | 133G_176D |

In some embodiments, the antigen-binding polypeptide construct of K-L cluster 7 comprises a combination of amino acid substitutions having one or more driver sets selected from a disulfide steering driver set, a steric 2 driver set, and a variable domain driver set.

In one embodiment, the amino acid combinations of K-L cluster 7 comprise one or more secondary substitutions selected from Table F.

In one embodiment, the antigen-binding polypeptide construct comprises a combination of amino acid substitutions according to K-L cluster 7, as set forth in one or more of the designs set forth in Table 10-A7.

### K-L Cluster 8:

In one embodiment, the antigen-binding polypeptide construct comprises a combination of amino acid substitutions according to K-L cluster 8 wherein:
H2 comprises amino acid substitution at position 143; L2 comprises amino acid substitution at position 124; and
a) H1 comprises amino acid substitution at position 186 or 179, and L1 comprises amino acid substitution at position 180;
b) H1 comprises amino acid substitution at position 186; and L1 comprises amino acid substitutions at position 133;
c) H1 comprises amino acid substitution at position 143; and L1 comprises amino acid substitutions at position 133; or
d) H1 comprises amino acid substitution at position 188; and L1 comprises amino acid substitutions at position 178.

In some embodiments, H1 further comprises amino acid substitutions at one or more of positions 124, 139, 177, and 190, L1 further comprises amino acid substitution at one or more of positions 129, 131, 135, and 176, H2 further comprises amino acid substitution at one or more of positions 122, 124, 145, 179, 186, 188, 39, and 45, and/or L2 further comprises amino acid substitutions at one or more of positions 129, 133, 135, 160, 176, 178, 38, and 44.

In one embodiment, the antigen-binding polypeptide construct comprises a combination of amino acid substitutions according to K-L cluster 8 wherein: H1 comprises amino acid substitution at positions 143, 186, 179 and/or 188; L1 comprises amino acid substitution at positions 129 and/or 178; and H2 comprises amino acid substitution at position 143, and L2 comprises amino acid substitution at position 124. In some embodiments, H1 further comprises amino acid substitutions at one or more of positions 124, 139, 177, and 190, L1 further comprises amino acid substitution at one or more of positions 131, 133, 135, 176, and 180, H2 further comprises amino acid substitution at one or more of positions 122, 124, 145, 179, 186, 188, 39, and 45, and/or L2 further comprises amino acid substitutions at one or more of positions 129, 133, 135, 160, 176, 178, 38, and 44.

In some embodiments, the antigen-binding polypeptide construct comprises a combination of amino acid substitutions according to K-L cluster 8 wherein H1 comprises amino acid substitutions at positions 124_143, 139_143, 139_143_186, 139_186, 139_188, 143, 143_179, 143_186, 143_186_188, 143_190, 177_188, 179, 179_190, 186, or 186_188, 188; L1 comprises amino acid substitutions at positions 129_131_133, 129_133, 129_133_135, 129_133_135_180, 129_133_178, 129_133_180, 129_176_178, 129_176_178_180, 129_178, 129_178_180, 129_180, 133_176_178, 133_178, or 176_178; H2 comprises amino acid substitutions at positions 122_143_145, 122_143_145_179, 124_143_145, 124_143_145_179, 143_145, 143_145_179, 143_145_179_186_188, 143_145_179_188, 143_145_188, 39_143_145_179, or 45_143_145_179, and L2 comprises amino acid substitutions at positions 124_129_160_178, 124_129_178, 124_133_178, 124_135_160_178, 124_135_178, 124_160_176_178, 124_160_178, 124_176_178, 124_178, 38 124_178, or 44_124_178.

In some embodiments, the antigen-binding polypeptide construct comprises a combination of amino acid substitutions according to K-L cluster 8 wherein the amino acid substitutions in H1 are selected from 124K, 139W, 143A, 143I, 143K, 143S, 177I, 179K, 186K, 186R, 188K, 188T, 190K, and conservative substitutions thereof; the amino acid substitutions in L1 are selected from 129T, 131D, 131E, 133D, 133L, 133W, 135S, 176A, 176D, 176E, 178D, 178E, 178T, 178W, 180E, and conservative substitutions thereof; the amino acid substitutions in H2 are selected from 122C, 124W, 143E, 145T, 179E, 186I, 188L, 188W, 39E, 45P, and conservative substitutions thereof; the amino acid substitutions in L2 are selected from 124C, 124K, 124R, 129K, 133A, 135W, 160K, 160R, 176A, 178R, 38R, 44F, and conservative substitutions thereof.

In some embodiments, the antigen-binding polypeptide construct has an H1L1 heterodimer comprising a combination of amino acid substitutions according to K-L cluster 8, wherein the HILL heterodimer comprises one of the following sets of amino acid substitutions:

| H1 | L1 |
|---|---|
| 143A_179K | 129T_133W_180E |
| 179K | 129T_180E |
| 179K | 129T_178E_180E |
| 179K_190K | 129T_133D_135S_180E |
| 143K_190K | 129T_133D_135S |
| 124K_143K | 129T_131E_133D |
| 124K_143K | 129T_131D_133D |
| 139W_143K | 129T_133D |
| 143K | 129T_131E/D_133D |
| 143A_186R | 129T_133W_180E |
| 186R | 129T_176A_178W_180E |
| 186R | 129T_180E |
| 186K | 129T_133D_178T |
| 139W_186K | 129T_133D_178T |
| 139W_143I_186K | 129T_133D_178T |
| 186K_188T | 133D_178T |
| 143S_186K_188T | 133D_178T |
| 188K | 129T_176D_178T |
| 139W_188K | 129T_176E_178E |
| 188K | 129T_178D |
| 188K | 129T_178E |
| 188K | 176E_178E |
| 177I_188K | 133L_176D_178E |

In further embodiments, the antigen-binding polypeptide construct has an H2L2 heterodimer comprising a combination of amino acid substitutions according to K-L cluster 8, wherein the H2L2 heterodimer comprises one of the following sets of amino acid substitutions:

| H2 | L2 |
|---|---|
| 122C_143E_145T | 124C_160K_178R |
| 122C_143E_145T_179E | 124C_160K_178R |
| 124W_143E_145T | 124K_133A_178R |
| 124W_143E_145T_179E | 124K_133A_178R |
| 143E_145T | 124K_178R |
| 143E_145T | 124R_160K_178R |
| 143E_145T | 124R_129K_160K_178R |
| 143E_145T_179E | 124R_135W_160K_178R |
| 143E_145T_179E | 124R_135W_178R |
| 143E_145T_179E | 124R_160K_178R |
| 143E_145T_179E | 124R_160R_178R |
| 143E_145T_179E | 124R_129K_160K_178R |
| 143E_145T_179E | 124R_129K_178R |
| 143E_145T_179E | 124R_178R |
| 45P_143E_145T_179E | 44F_124K_178R |
| 45P_143E_145T_179E | 44F_124R_178R |
| 39E_143E_145T_179E | 38R_124K_178R |
| 39E_143E_145T_179E | 38R_124R_178R |
| 143E_145T_179E_186I_188W | 124R_176A_178R |
| 143E_145T_179E_186I_188W | 124R_160R_176A_178R |
| 143E_145T_179E_188L | 124K_178R |
| 143E_145T_179E_188L | 124R_160K_178R |
| 143E_145T_179E_188L | 124R_178R |
| 143E_145T_179E_188W | 124R_176A_178R |
| 143E_145T_188W | 124R_160R_176A_178R |
| 143E_145T_188L | 124R_178R |
| 143E_145T_188L | 124R_160K_178R |

In some embodiments, the antigen-binding polypeptide construct comprises a combination of one H1L1 heterodimer with one H2L2 heterodimer. In one embodiment, the combination of H1L1 heterodimer and H2L2 heterodimer is one of the following:

| H1 | L1 | H2 | L2 |
|---|---|---|---|
| 143K | 129T_133D | 124W_143E_145T_179E | 124K_133A_178R |
| 179K | 129T_180E | 143E_145T_188L | 124R_160K_178R |
| 186R | 129T_180E | 45P_143E_145T_179E | 44F_124K_178R |
| 139W_143K | 129T_133D | 143E_145T_179E | 124R_135W_178R |
| 179K | 129T_178E_180E | 143E_145T_179E | 124R_178R |

In some embodiments, the antigen-binding polypeptide construct comprises a combination of amino acid substitutions according to K-L cluster 8 having one or more of steric 2, steric 3, steric 4, and variable domain driver sets. In one embodiment, the antigen-binding polypeptide construct comprises a combination of amino acid substitutions according to K-L cluster 8 that introduce a non-naturally occurring disulphide bond.

In one embodiment, the amino acid combinations of K-L cluster 8 comprise one or more secondary substitutions selected from Table F.

In one embodiment, the antigen-binding polypeptide construct comprises a combination of amino acid substitutions according to K-L cluster 8, as set forth in one or more of the designs in Table 10-A8.

### K-L Cluster 9:

In one embodiment, the antigen-binding polypeptide construct comprises a combination of amino acid substitutions according to K-L cluster 9 wherein:
H1 comprises amino acid substitution at position 179, 186, 143 and/or 188; L1 comprises amino acid substitution at position 180, 133 and/or 176_178; H2 comprises amino acid substitution at position 143, and L2 comprises amino acid substitution at position 131 and/or 124. In some embodiments, H1 further comprises amino acid substitutions at position 125, L1 further comprises amino acid substitution at position 122 or 129, H2 further comprises amino acid substitution at one or more of positions 145, 179 and 228, and/or L2 further comprises amino acid substitutions at one or more of positions 121, 129, 135, 160, and 178.

In one embodiment, the antigen-binding polypeptide construct comprises a combination of amino acid substitutions according to K-L cluster 9 wherein: H1 comprises amino acid substitution at position 125; L1 comprises amino acid substitution at positions 122_129; and H2 comprises amino acid substitution at position 145, and L2 comprises amino acid substitution at position 121 and/or 124. In some embodiments, H1 further comprises amino acid substitutions at one or more of positions 143, 179, 186, and 188, L1 further comprises amino acid substitution at one or more of positions, 133, 176, 178, and 180, H2 further comprises amino acid substitution at one or more of positions 143, 179, and 228, and/or L2 further comprises amino acid substitutions at one or more of positions 129, 131, 135, 160, and 178.

In some embodiments, the antigen-binding polypeptide construct comprises a combination of amino acid substitutions according to K-L cluster 9 wherein H1 comprises amino acid substitutions at positions 125_143, 125_179, 125_186, or 125_188; L1 comprises amino acid substitutions at positions 122_129_133, 122_129_176_178, or 122_129_180; H2 comprises amino acid substitutions at positions 143_145, 143_145_179, 143_145_179_228, 143_145_228, or 145_179_228, and L2 comprises amino acid substitutions at positions 121_124_160_178, 121_124_178, 121_129_131, 121_131, 124_135_160_178, or 124_135_178.

In some embodiments, the antigen-binding polypeptide construct comprises a combination of amino acid substitutions according to K-L cluster 9 wherein the amino acid substitutions in H1 are selected from 125R, 143K, 179K, 186R, 188K, and conservative substitutions thereof; the amino acid substitutions in L1 are selected from 122D, 129T, 133D, 176D, 176E, 178E, 178T, 180E, and conservative substitutions thereof; the amino acid substitutions in H2 are selected from 143E, 145T, 179E, 228D, and conservative substitutions thereof; the amino acid substitutions in L2 are selected from 135W, 124R, 160K, 121K, 131K, 129K, 178R, and conservative substitutions thereof.

In some embodiments, the antigen-binding polypeptide construct has an H1L1 heterodimer comprising a combination of amino acid substitutions according to K-L cluster 9, wherein the HILL heterodimer comprises one of the following sets of amino acid substitutions:

| H1 | L1 |
|---|---|
| 125R_143K | 122D_129T_133D |
| 125R_179K | 122D_129T_180E |
| 125R_186R | 122D_129T_180E |
| 125R_188K | 122D_129T_176D_178T |
| 125R_188K | 122D_129T_176E_178E |

In further embodiments, the antigen-binding polypeptide construct has an H2L2 heterodimer comprising a combination of amino acid substitutions according to K-L cluster 9, wherein the H2L2 heterodimer comprises one of the following sets of amino acid substitutions:

| H2 | L2 |
|---|---|
| 143E_145T | 124R_135W_160K_178R |
| 143E_145T | 124R_135W_178R |
| 143E_145T_228D | 121K_124R_160K_178R |
| 143E_145T_228D | 121K_124R_178R |
| 145T_179E_228D | 121K_129K_131K |
| 145T_179E_228D | 121K_131K |
| 143E_145T_179E | 124R_135W_160K_178R |
| 143E_145T_179E | 124R_135W_160K_178R |
| 143E_145T_179E_228D | 121K_124R_178R |
| 143E_145T_179E_228D | 121K_124R_160K_178R |

In some embodiments, the antigen-binding polypeptide construct comprises a combination of one H1L1 heterodimer with one H2L2 heterodimer. In one embodiment, the combination of H1L1 heterodimer and H2L2 heterodimer is one of the following:

| H1 | L1 | H2 | L2 |
|---|---|---|---|
| 125R_179K | 122D_129T_180E | 143E_145T_228D | 121K_124R_178R |
| 125R_188K | 122D_129T_176E_178E | 143E_145T_179E_228D | 121K_124R_178R |

In some embodiments, the antigen-binding polypeptide construct comprises a combination of amino acid substitutions according to K-L cluster 9, having a disulfide steering driver set.

In one embodiment, the amino acid combinations of K-L cluster 9 comprise one or more secondary substitutions selected from Table F.

In one embodiment, the antigen-binding polypeptide construct comprises a combination of amino acid substitutions according to K-L cluster 9, as set forth in one or more of the designs in Table 10-A9.

### K-L Cluster 10:

In one embodiment, the antigen-binding polypeptide construct comprises a combination of amino acid substitutions according to K-L cluster 10 wherein: H1 comprises amino acid substitution at position 174, 179 or 186; L1 comprises amino acid substitution at position 176 or 180; H2 comprises amino acid substitution at position 143 or 190, and L2 comprises amino acid substitution at position 131, 135 or 124; or H1 comprises amino acid substitution at position 174; L1 comprises amino acid substitution at position 176; H2 comprises amino acid substitution at position 190; and L2 comprises no amino acid substitutions that promote preferential pairing or comprises amino acid substitution at position 135. In some embodiments, H1 further comprises amino acid substitutions at position 143, L1 further comprises amino acid substitution at one or more of positions 116, 129 and 133, H2 further comprises amino acid substitution at one or more of positions 145, 179, and 188, and/or L2 further comprises amino acid substitutions at one or more of positions 133, 160 and 178.

In one embodiment, the antigen-binding polypeptide construct comprises a combination of amino acid substitutions according to K-L cluster 10 wherein: H1 comprises amino acid substitution at positions 174 and/or 186; L1 comprises amino acid substitution at positions 176 and/or 180; and H2 comprises amino acid substitution at position 145, 190 and/or 188, and L2 comprises amino acid substitution at positions 135, 131, 178, or 133 or comprises no amino acid substitutions that promote preferential pairing. In some embodiments, H1 further comprises amino acid substitutions at one or more of positions 143 and/or 179, L1 further comprises amino acid substitution at one or more of positions 116, 129, and 133, H2 further comprises amino acid substitution at positions 143 and/or 179, and/or L2 further comprises amino acid substitutions at positions 124 and/or 160.

In some embodiments, the antigen-binding polypeptide construct comprises a combination of amino acid substitutions according to K-L cluster 10 wherein H1 comprises amino acid substitutions at positions 143_174, 174, 174_179, 174_186, or 186; L1 comprises amino acid substitutions at positions 116_129_133_176, 116_129_176_180, 116_176, 129_180, or 176; H2 comprises amino acid substitutions at positions 143_145_179_188_190, 143_145_179_190, 143_145_190, 143_190, 145_179, 145_179_188_190, 188, or 190, and L2 comprises amino acid substitutions at positions 124_135_160_178, 124_135_178, 131, 131_135, 133, 135, 135_178, 178, or comprises no amino acid substitutions that promote preferential pairing.

In some embodiments, the antigen-binding polypeptide construct comprises a combination of amino acid substitutions according to K-L cluster 10 wherein the amino acid substitutions in H1 are selected from 143K, 174G, 179K, 186R, and conservative substitutions thereof; the amino acid substitutions in L1 are selected from 116F, 129T, 133D, 176F, 180E, and conservative substitutions thereof; the amino acid substitutions in H2 are selected from 143E, 143I, 145T, 179E, 188F, 190F, and conservative substitutions thereof; the amino acid substitutions in L2 are selected from 124K, 124R, 131K, 133A, 135A, 160K, 178F, 178R, and conservative substitutions thereof.

In some embodiments, the antigen-binding polypeptide construct has an H1L1 heterodimer comprising a combination of amino acid substitutions according to K-L cluster 10, wherein the H1L1 heterodimer comprises one of the following sets of amino acid substitutions:

| H1 | L1 |
|---|---|
| 143K_174G | 116F_129T_133D_176F |
| 186R | 129T_180E |
| 174G_179K | 116F_129T_176F_180E |
| 174G_186R | 116F_129T_176F_180E |
| 174G | 176F |
| 174G | 116F_176F |

In further embodiments, the antigen-binding polypeptide construct has an H2L2 heterodimer comprising a combination of amino acid substitutions according to K-L cluster 10, wherein the H2L2 heterodimer comprises one of the following sets of amino acid substitutions:

| H2 | L2 |
|---|---|
| 145T_179E | 131K |
| 145T_179E_188F_190F | 131K_135A |
| 143I_190F | 135A |
| 143I_190F | — |
| 143I_190F | 178F |
| 188F | 133A |
| 190F | — |
| 190F | 135A |
| 190F | 135A_178F |
| 190F | 178F |
| 143E_145T_179E_188F_190F | 124K_135A_178R |
| 143E_145T_179E_190F | 124R_135A_178R |
| 143E_145T_179E_190F | 124K_135A_178R |
| 143E_145T_179E_190F | 124R_135A_160K_178R |
| 143E_145T_190F | 124R_135A_160K_178R |

In some embodiments, the antigen-binding polypeptide construct comprises a combination of one H1L1 heterodimer with one H2L2 heterodimer. In one embodiment, the combination of H1L1 heterodimer and H2L2 heterodimer is one of the following:

| H1 | L1 | H2 | L2 |
|---|---|---|---|
| 143K_174G | 116F_129T_133D_176F | 143E_145T_179E_190F | 124R_135A_178R |
| 174G_179K | 116F_129T_176F_180E | 143E_145T_179E_190F | 124K_135A_178R |

In some embodiments, the antigen-binding polypeptide construct comprises a combination of amino acid substitutions according to K-L cluster 10, having a steric 1 driver set.

In one embodiment, the amino acid combinations of K-L cluster 10 comprise one or more secondary substitutions selected from Table F.

In one embodiment, the antigen-binding polypeptide construct comprises a combination of amino acid substitutions according to K-L cluster 10, as set forth in one or more of the designs in Table 10-A10.

### K-L Cluster 11:

In one embodiment, the antigen-binding polypeptide construct comprises a combination of amino acid substitutions according to K-L cluster 11 wherein: H1 comprises amino acid substitution at positions 143_190; L1 comprises amino acid substitution at position 133; H2 comprises amino acid substitution at position 124, and L2 comprises amino acid substitution at positions 131_135. In some embodiments, H1 further comprises amino acid substitutions at position 125, L1 further comprises amino acid substitution at one or more of positions 122, 129 and 135, H2 further comprises amino acid substitution at one or more of positions 139, 145, 190, and 228, and/or L2 further comprises amino acid substitutions at position 121.

In one embodiment, the antigen-binding polypeptide construct comprises a combination of amino acid substitutions according to K-L cluster 11 wherein: H1 comprises amino acid substitution at positions 143_190; L1 comprises amino acid substitution at positions 129_133_135; and H2 comprises amino acid substitution at position 124_145, and L2 comprises amino acid substitution at positions 131_135. In some embodiments, H1 further comprises amino acid substitutions at position 125, L1 further comprises amino acid substitution at position 122, H2 further comprises amino acid substitution at one or more of positions 139, 190 and 228, and/or L2 further comprises amino acid substitutions at position 121.

In some embodiments, the antigen-binding polypeptide construct comprises a combination of amino acid substitutions according to K-L cluster 11 wherein H1 comprises amino acid substitutions at positions 125_143_190, or 143_190; L1 comprises amino acid substitutions at positions 122_129_133_135, or 129_133_135; H2 comprises amino acid substitutions at positions 124_139_145_190, 124_139_145_190_228, or 124_145, and L2 comprises amino acid substitutions at positions 121_131_135, or 131_135.

In some embodiments, the antigen-binding polypeptide construct comprises a combination of amino acid substitutions according to K-L cluster 11 wherein the amino acid substitutions in H1 are selected from 125R, 143K, 190K, and conservative substitutions thereof; the amino acid substitutions in L1 are selected from 122D, 129T, 133D, 135S, and conservative substitutions thereof; the amino acid substitutions in H2 are selected from 124E, 139I, 145T, 190I, 228D, and conservative substitutions thereof; the amino acid substitutions in L2 are selected from 121K, 131K, 135K, and conservative substitutions thereof.

In some embodiments, the antigen-binding polypeptide construct has an H1L1 heterodimer comprising a combination of amino acid substitutions according to K-L cluster 11, wherein the H1L1 heterodimer comprises one of the following sets of amino acid substitutions:

| H1 | L1 |
|---|---|
| 143K_190K | 129T_133D_135S |
| 125R_143K_190K | 122D_129T_133D_135S |

In further embodiments, the antigen-binding polypeptide construct has an H2L2 heterodimer comprising a combination of amino acid substitutions according to K-L cluster 11, wherein the H2L2 heterodimer comprises one of the following sets of amino acid substitutions:

| H2 | L2 |
|---|---|
| 124E_139I_145T_190I | 131K_135K |
| 124E_139I_145T_190I | 131K_135K |
| 124E_139I_145T_190I_228D | 121K_131K_135K |

In some embodiments, the antigen-binding polypeptide construct comprises a combination of one H1L1 heterodimer with one H2L2 heterodimer. In one embodiment, the combination of H1L1 heterodimer and H2L2 heterodimer is one in which H1 comprises 143K_190K, L1 comprises 129T_133D_135S, H2 comprises 124E_145T, and L2 comprises 131K_135K.

In some embodiments, the antigen-binding polypeptide construct comprises a combination of amino acid substitutions according to K-L cluster 11, having a disulfide steering driver set.

In one embodiment, the amino acid combinations of K-L cluster 11 comprise one or more secondary substitutions selected from Table F.

In one embodiment, the antigen-binding polypeptide construct comprises a combination of amino acid substitutions according to K-L cluster 11, as set forth in one or more of the designs in Table 10-A11.

### K-L Cluster 12:

In one embodiment, the antigen-binding polypeptide construct comprises a combination of amino acid substitutions according to K-L cluster 12 wherein: H1 comprises amino acid substitution at position 143 and/or 186; L1 comprises amino acid substitution at position 133; H2 comprises amino acid substitution at position 124, and L2 comprises amino acid substitution at position 131. In some embodiments, H1 further comprises amino acid substitutions at one or more of positions 124, 125, 139, and 188, L1 further comprises amino acid substitution at one or more of positions 122, 129, 131, and 178, H2 further comprises amino acid substitution at one or more of positions 143, 145, 179, 186, 188, 228, 39, and 45, and/or L2 further comprises amino acid substitutions at one or more of positions 121, 133, 135, 176, 178, 38, and 44.

In some embodiments, the antigen-binding polypeptide construct comprises a combination of amino acid substitutions according to K-L cluster 12 wherein H1 comprises amino acid substitutions at positions 124_143, 125_143, 125_143_186, 125_186, 125_186_188, 139_143, 139_143_186, 139_186, 139_186_188, 143, or 186_188; L1 comprises amino acid substitutions at positions 122_129_133, 122_129_133_178, 122_133_178, 129_131_133, 129_133, 129_133_178, or 133_178; H2 comprises amino acid substitutions at positions 124_143_145, 124_145_179, 124_145_179_186_188, 124_145_179_188, 124_145_179_228, 124_145_186, 39_124_145_179, or 45_124_145_179, and L2 comprises amino acid substitutions at positions 121_131_133_176, 131_133_135, 131_133_135_176, 131_133_135_178, 131_133_176, 131_133_176_178, 38_131_133_176, or 44_131_133_176.

In some embodiments, the antigen-binding polypeptide construct comprises a combination of amino acid substitutions according to K-L cluster 12 wherein the amino acid substitutions in H1 are selected from 124K, 125R, 139W, 143I, 143K, 186K, 188T, and conservative substitutions thereof; the amino acid substitutions in L1 are selected from 122D, 129T, 131D, 131E, 133D, 178T, and conservative substitutions thereof; the amino acid substitutions in H2 are selected from 124E, 143E, 145T, 179E, 186E, 186I, 188W, 228D, 39E, 45P, and conservative substitutions thereof; the amino acid substitutions in L2 are selected from 121K, 131K, 131R, 133G, 133S, 133T, 135K, 135W, 176R, 178A, 178S, 38R, 44F, and conservative substitutions thereof.

In some embodiments, the antigen-binding polypeptide construct has an H1L1 heterodimer comprising a combination of amino acid substitutions according to K-L cluster 12, wherein the H1L1 heterodimer comprises one of the following sets of amino acid substitutions:

| H1 | L1 |
|---|---|
| 143K | 129T_133D |
| 143K | 129T_131D_133D |
| 143K | 129T_131E_133D |
| 124K_143K | 129T_131D_133D |
| 124K_143K | 129T_131E_133D |
| 139W_143K | 129T_133D |
| 125R_143K | 122D_129T_133D |
| 125R_143I_186K | 122D_129T_133D_178T |
| 125R_186K | 122D_129T_133D_178T |
| 125R_186K_188T | 122D_133D_178T |
| 139W_143I_186K | 129T_133D_178T |
| 139W_186K | 129T_133D_178T |
| 139W_186K_188T | 133D_178T |
| 186K_188T | 133D_178T |

In further embodiments, the antigen-binding polypeptide construct has an H2L2 heterodimer comprising a combination of amino acid substitutions according to K-L cluster 12, wherein the H2L2 heterodimer comprises one of the following sets of amino acid substitutions:

| H2 | L2 |
|---|---|
| 124E_145T_179E | 121K_131R_133G_176R |
| 124E_145T_179E | 131K_133G_176R |
| 124E_145T_179E | 131K_133G_176R_178A |
| 124E_145T_179E | 131R_133G_135W_176R |
| 124E_145T_179E | 131R_133G_176R |
| 124E_145T_179E | 131R_133G_176R_178A |
| 124E_145T_186E | 131R_133S_135K |
| 124E_143E_145T | 131R_133T_135K_178S |
| 45P_124E_145T_179E | 44F_131R_133G_176R |
| 39E_124E_145T_179E | 38R_131R_133G_176R |

In some embodiments, the antigen-binding polypeptide construct comprises a combination of one H1L1 heterodimer with one H2L2 heterodimer. In one embodiment, the combination of H1L1 heterodimer and H2L2 heterodimer is one of the following:

| H1 | L1 | H2 | L2 |
|---|---|---|---|
| 143K | 129T_133D | 124E_145T_179E | 131R_133G_176R |
| 186K_188T | 133D_178T | 124E_145T_179E | 131R_133G_176R |
| 139W_143K | 129T_133D | 124E_145T_179E | 131R_133G_135W_176R |

In some embodiments, the antigen-binding polypeptide construct comprises a combination of amino acid substitutions according to K-L cluster 12, having one or more of disulfide steering, steric 2, steric 3, variable domain electrostatic and variable domain steric driver sets.

In one embodiment, the amino acid combinations of K-L cluster 12 comprise one or more secondary substitutions selected from Table F.

In one embodiment, the antigen-binding polypeptide construct comprises a combination of amino acid substitutions according to K-L cluster 12, as set forth in one or more of the designs in Table 10-A12.

### K-K Cluster 1:

In one embodiment, the antigen-binding polypeptide construct comprises a combination of amino acid substitutions according to K-K cluster 1 wherein: H1 comprises amino acid substitution at positions 143_179; L1 comprises amino acid substitution at positions 124_178; H2 comprises amino acid substitution at position 186, and L2 comprises amino acid substitution at positions 178_180 or 160_180. In some embodiments, H1 further comprises amino acid substitution at position 145.

In one embodiment, the antigen-binding polypeptide construct comprises a combination of amino acid substitutions according to K-K cluster 1 wherein: H1 comprises amino acid substitution at positions 143_145_179; L1 comprises amino acid substitution at positions 124_178; H2 comprises amino acid substitution at position 186, and L2 comprises amino acid substitution at position 180. In some embodiments, L2 further comprises amino acid substitutions at position 160 and/or 178.

In some embodiments, the antigen-binding polypeptide construct comprises a combination of amino acid substitutions according to K-K cluster 1 wherein H1 comprises amino acid substitutions at position 143_145_179; L1 comprises amino acid substitutions at positions 124_178; H2 comprises amino acid substitutions at position 186, and L2 comprises amino acid substitutions at positions 178_180 or 160_180.

In some embodiments, the antigen-binding polypeptide construct comprises a combination of amino acid substitutions according to K-K cluster 1 wherein the amino acid substitutions in H1 are selected from 143E, 145T, 179E, and conservative substitutions thereof; the amino acid substitutions in L1 are selected from 124K, 178R, and conservative substitutions thereof; the amino acid substitution in H2 is 186R, or conservative substitution thereof; the amino acid substitutions in L2 are selected from 160E, 178E, 180E, and conservative substitutions thereof.

In some embodiments, the antigen-binding polypeptide construct has an H1L1 heterodimer comprising a combination of amino acid substitutions according to K-K cluster 1, wherein the H1L1 heterodimer comprises the following set of amino acid substitutions: H1 comprises 143E_145T_179E and L1 comprises 124K_178R. In a further embodiment, the antigen-binding polypeptide construct has an H2L2 heterodimer comprising a combination of amino acid substitutions according to K-K cluster 1, wherein the H2L2 heterodimer comprises the following sets of amino acid substitutions:H2 comprises 124K_178R and L2 comprises 178E_180E or 160E_180E. In some embodiments, the antigen-binding polypeptide construct comprises a combination of these H1L1 and H2L2 heterodimers.

In one embodiment, the antigen-binding polypeptide construct comprises a combination of amino acid substitutions according to K-K cluster 1 as set forth in Table 10-B1.

### K-K Cluster 2:

In one embodiment, the antigen-binding polypeptide construct comprises a combination of amino acid substitutions according to K-K cluster 2 wherein: H1 comprises amino acid substitution at position 143; L1 comprises amino acid substitution at position 124; H2 comprises amino acid substitution at position 179 or 186, and L2 comprises amino acid substitution at position 124_160_180. In some embodiments, H1 further comprises amino acid substitution at position 145, and/or H2 further comprises amino acid substitution at position 146.

In one embodiment, the antigen-binding polypeptide construct comprises a combination of amino acid substitutions according to K-K cluster 2 wherein: H1 comprises amino acid substitution at positions 143_145; L1 comprises amino acid substitution at position 124; and H2 comprises amino acid substitution at positions 179 or 186, and L2 comprises amino acid substitution at positions 124_160_180. In some embodiments, H2 further comprises amino acid substitution at position 146.

In some embodiments, the antigen-binding polypeptide construct comprises a combination of amino acid substitutions according to K-K cluster 2 wherein H1 comprises amino acid substitutions at positions 143_145; L1 comprises amino acid substitutions at position 124; H2 comprises amino acid substitutions at position 186, 179, or 146_179, and L2 comprises amino acid substitutions at positions 124_160_180.

In some embodiments, the antigen-binding polypeptide construct comprises a combination of amino acid substitutions according to K-K cluster 2 wherein the amino acid substitutions in H1 are selected from 143E, 145T, and conservative substitutions thereof; the amino acid substitutions in L1 is 124R, or conservative substitutions thereof; the amino acid substitutions in H2 are selected from 186R, 179K, 146G, and conservative substitutions thereof; the amino acid substitutions in L2 are selected from 124E, 160E, 180E, and conservative substitutions thereof.

In some embodiments, the antigen-binding polypeptide construct has an H1L1 heterodimer comprising a combination of amino acid substitutions according to K-K cluster 2, wherein the H1L1 heterodimer comprises the following set of amino acid substitutions: H1 comprises 143E_145T and L1 comprises 124R. In a further embodiment, the antigen-binding polypeptide construct has an H2L2 heterodimer comprising a combination of amino acid substitutions according to K-K cluster 2, wherein the H2L2 heterodimer comprises the following set of amino acid substitutions:H2 comprises 186R, 179K or 146G_179K and L2 comprises 124E_160E_180E. In some embodiments, the antigen-binding polypeptide construct comprises a combination of one H1L1 heterodimer with one H2L2 heterodimer. In one embodiment, the combination of H1L1 heterodimer and H2L2 heterodimer is one wherein H1 comprises 143E_145T, L1 comprises 124R, H2 comprises 179K, and L2 comprises 124E_160E_180E.

In one embodiment, the amino acid combination of K-K cluster 2 comprises one or more secondary substitutions selected from Table F.

In one embodiment, the antigen-binding polypeptide construct comprises a combination of amino acid substitutions according to K-K cluster 2, as set forth in one or more of the designs in Table 10-B2.

### K-K Cluster 3:

In one embodiment, the antigen-binding polypeptide construct comprises a combination of amino acid substitutions according to K-K cluster 3 wherein:
H1 comprises amino acid substitution at position 186; L1 comprises amino acid substitution at positions 180 or 178_180; H2 comprises amino acid substitution at position 143 and/or 179, and L2 comprises amino acid substitution at positions 124_178 or 131. In some embodiments, H2 further comprises amino acid substitution at position 145.

In one embodiment, the antigen-binding polypeptide construct comprises a combination of amino acid substitutions according to K-K cluster 3 wherein: H1 comprises amino acid substitution at position 186; L1 comprises amino acid substitution at position 180; and H2 comprises amino acid substitution at position 145, and L2 comprises amino acid substitution at position 124 or 131. In some embodiments, L1 further comprises amino acid substitution at position 178, H2 further comprises amino acid substitution at positions 143 and/or 179, and/or L2 further comprises amino acid substitutions at position 178.

In some embodiments, the antigen-binding polypeptide construct comprises a combination of amino acid substitutions according to K-K cluster 3 wherein H1 comprises amino acid substitutions at position 186; L1 comprises amino acid substitutions at position 180 or 178_180; H2 comprises amino acid substitutions at positions 143_145, 143_145_179, or 145_179, and L2 comprises amino acid substitutions at positions 131 or 124_178.

In some embodiments, the antigen-binding polypeptide construct comprises a combination of amino acid substitutions according to K-K cluster 3 wherein the amino acid substitutions in H1 are selected from 186R, and conservative substitutions thereof; the amino acid substitutions in L1 are selected from 178E, 180E, and conservative substitutions thereof; the amino acid substitutions in H2 are selected from 143E, 145T, 179E, and conservative substitutions thereof; the amino acid substitutions in L2 are selected from 124K, 131K, 178R, and conservative substitutions thereof.

In some embodiments, the antigen-binding polypeptide construct has an H1L1 heterodimer comprising a combination of amino acid substitutions according to K-K cluster 3, wherein the H1L1 heterodimer comprises the following set of amino acid substitutions: H1 comprises 186R and L1 comprises 178E_180E or 180E. In further embodiments, the antigen-binding polypeptide construct has an H2L2 heterodimer comprising a combination of amino acid substitutions according to K-K cluster 3, wherein the H2L2 heterodimer comprises one of the following sets of amino acid substitutions:

| H2 | L2 |
|---|---|
| 143E_145T_179E | 124K_178R |
| 143E_145T | — |
| 145T_179E | 131K |

In some embodiments, the antigen-binding polypeptide construct comprises a combination of one H1L1 heterodimer with one H2L2 heterodimer. In one embodiment, the combination of H1L1 heterodimer and H2L2 heterodimer is one where H1 comprises 186R, L1 comprises 180E, H2 comprises 143E_145T_179E, and L2 comprises 124K_178R.

In one embodiment, the amino acid combinations of K-K cluster 3 comprise one or more secondary substitutions selected from Table F.

In one embodiment, the antigen-binding polypeptide construct comprises a combination of amino acid substitutions according to K-K cluster 3 as set forth in one or more of the designs in Table 10-B3.

### K-K Cluster 4:

In one embodiment, the antigen-binding polypeptide construct comprises a combination of amino acid substitutions according to K-K cluster 4 wherein: H1 comprises amino acid substitution at position 179; L1 comprises amino acid substitution at position 180; H2 comprises amino acid substitution at position 143, and L2 comprises amino acid substitution at position 124. In some embodiments, H1 further comprises amino acid substitutions at position 146, H2 further comprises amino acid substitution at position 145, and/or L2 further comprises amino acid substitutions at position 160 and/or 178.

In one embodiment, the antigen-binding polypeptide construct comprises a combination of amino acid substitutions according to K-K cluster 4 wherein: H1 comprises amino acid substitution at position 179; L1 comprises amino acid substitution at position 180; H2 comprises amino acid substitution at positions 143_145, and L2 comprises amino acid substitution at position 124. In some embodiments, H1 further comprises amino acid substitutions at position 146, and/or L2 further comprises amino acid substitutions at position 160 and/or 178.

In some embodiments, the antigen-binding polypeptide construct comprises a combination of amino acid substitutions according to K-K cluster 4 wherein H1 comprises amino acid substitutions at positions 146_179 or 179; L1 comprises amino acid substitutions at position 180; H2 comprises amino acid substitutions at positions 143_145, and L2 comprises amino acid substitutions at positions 124 or 124_160_178.

In some embodiments, the antigen-binding polypeptide construct comprises a combination of amino acid substitutions according to K-K cluster 4 wherein the amino acid substitutions in H1 are selected from 146G, 179K, and conservative substitutions thereof; the amino acid substitutions in L1 is 180E, or conservative substitutions thereof; the amino acid substitutions in H2 are selected from 143E, 145T, and conservative substitutions thereof; the amino acid substitutions in L2 are selected from 124R, 160K, 178R, and conservative substitutions thereof.

In some embodiments, the antigen-binding polypeptide construct has an H1L1 heterodimer comprising a combination of amino acid substitutions according to K-K cluster 4, wherein the HILL heterodimer comprises the following set of amino acid substitutions: H1 comprises 179K or 146G_179K, and L1 comprises 180E. In further embodiments, the antigen-binding polypeptide construct has an H2L2 heterodimer comprising a combination of amino acid substitutions according to K-K cluster 4, wherein the H2L2 heterodimer comprises the following sets of amino acid substitutions:H2 comprises 143E_145T and L2 comprises Q124R_Q160K_T178R or Q124R. In some embodiments, the antigen-binding polypeptide construct comprises a combination of one H1L1 heterodimer with one H2L2 heterodimer. In one embodiment, the combination of H1L1 heterodimer and H2L2 heterodimer is one wherein H1 comprises 179K, L1 comprises 180E, H2 comprises 143E_145T, and L2 comprises 124R_160K_178R.

In one embodiment, the amino acid combinations of K-K cluster 4 comprise one or more secondary substitutions selected from Table F.

In one embodiment, the antigen-binding polypeptide construct comprises a combination of amino acid substitutions according to K-K cluster 4 as set forth in one or more of the designs in Table 10-B4.

### K-K Cluster 5:

In one embodiment, the antigen-binding polypeptide construct comprises a combination of amino acid substitutions according to K-K cluster 5 wherein: H1 comprises amino acid substitution at position 143 or 186; L1 comprises amino acid substitution at position 180 or comprises no amino acid substitutions that promote preferential pairing; H2 comprises amino acid substitution at positions 143_145, and L2 comprises amino acid substitution at position 124. In some embodiments, L2 further comprises amino acid substitutions at one or more of positions 160 and/or 178. In an additional embodiment, L2 comprises amino acid substitutions at positions 124, 124_178 or 124_160_178.

In some embodiments, the antigen-binding polypeptide construct comprises a combination of amino acid substitutions according to K-K cluster 5 wherein the amino acid substitutions in H1 are selected from 186R, 143R, 143K, and conservative substitutions thereof; the amino acid substitution in L1 is 180E, and conservative substitutions thereof; the amino acid substitutions in H2 are selected from 143E, 145T, and conservative substitutions thereof; the amino acid substitutions in L2 are selected from 124R, 160K, 178R, and conservative substitutions thereof.

In some embodiments, the antigen-binding polypeptide construct has an H1L1 heterodimer comprising a combination of amino acid substitutions according to K-K cluster 5, wherein the H1L1 heterodimer comprises one of the following sets of amino acid substitutions:

| H1 | L1 |
|---|---|
| 186R | 180E |
| 186R | — |
| 143R/K | — |

In further embodiments, the antigen-binding polypeptide construct has an H2L2 heterodimer comprising a combination of amino acid substitutions according to K-K cluster 5, wherein the H2L2 heterodimer comprises one of the following sets of amino acid substitutions:

| H2 | L2 |
|---|---|
| 143E_145T | 124R_160K_178R |
| 143E_145T | 124R |
| 143E_145T | 124K_178R |

In some embodiments, the antigen-binding polypeptide construct comprises a combination of one H1L1 heterodimer with one H2L2 heterodimer.

In one embodiment, the amino acid combinations of K-K cluster 5 comprise one or more secondary substitutions selected from Table F.

In one embodiment, the antigen-binding polypeptide construct comprises a combination of amino acid substitutions according to K-K cluster 5 as set forth in one or more of the designs in Table 10-B5.

### K-K Cluster 6:

In one embodiment, the antigen-binding polypeptide construct comprises a combination of amino acid substitutions according to K-K cluster 6 wherein: H1 comprises amino acid substitution at position 39 or comprises no amino acid substitutions that promote preferential pairing; L1 comprises amino acid substitution at position 38 or comprises no amino acid substitutions that promote preferential pairing; H2 comprises amino acid substitution at position 39, and L2 comprises amino acid substitution at position 38.

In some embodiments, the antigen-binding polypeptide construct comprises a combination of amino acid substitutions according to K-K cluster 6 wherein the amino acid substitutions in H1 are selected from 39D, 39E, 39K, 39R, and conservative substitutions thereof; the amino acid substitutions in L1 are selected from 38D, 38E, 38K, 38R, and conservative substitutions thereof; the amino acid substitutions in H2 are selected from 39D, 39E, 39K, 39R, and conservative substitutions thereof; the amino acid substitutions in L2 are selected from 38D, 38E, 38K, 38R, and conservative substitutions thereof.

In some embodiments, the antigen-binding polypeptide construct has an H1L1 heterodimer comprising a combination of amino acid substitutions according to K-K cluster 6, wherein the HILL heterodimer comprises one of the following sets of amino acid substitutions:

| H1 | L1 |
|---|---|
| — | — |
| 39R | 38D/E |
| 39K | 38E/D |
| 39E | 38R/K |
| 39D | 38R |

In further embodiments, the antigen-binding polypeptide construct has an H2L2 heterodimer comprising a combination of amino acid substitutions according to K-K cluster 6, wherein the H2L2 heterodimer comprises one of the following sets of amino acid substitutions:

| H2 | L2 |
|---|---|
| 39D | 38R/K |
| 39R | 38E/D |
| 39K | 38E |
| 39E | 38R/K |

In some embodiments, the antigen-binding polypeptide construct comprises a combination of one H1L1 heterodimer with one H2L2 heterodimer. In one embodiment, the combination of H1L1 heterodimer and H2L2 heterodimer is one where H1 comprises 39R, L1 comprises 38E, H2 comprises 39D, and L2 comprises 38R.

In one embodiment, the antigen-binding polypeptide construct comprises a combination of amino acid substitutions according to K-K cluster 6 as set forth in one or more of the designs in Table 10-B6. In one embodiment, the K-K cluster 6 design is not the design corresponding to LCCA unique identifier 10674-10749 or 10679-10744.

### K-K Cluster 7:

In one embodiment, the antigen-binding polypeptide construct comprises a combination of amino acid substitutions according to K-K cluster 7 wherein: H1 comprises no amino acid substitutions that promote preferential pairing; L1 comprises amino acid substitution at position 135; H2 comprises amino acid substitution at position 139, and L2 comprises amino acid substitution at position 116. In some embodiments, L2 further comprises amino acid substitution at position 135.

In some embodiments, the antigen-binding polypeptide construct comprises a combination of amino acid substitutions according to K-K cluster 7 wherein the amino acid substitution in L1 is 135W, or conservative substitutions thereof; the amino acid substitution in H2 is 139W, or conservative substitutions thereof; and the amino acid substitutions in L2 are selected from 116A, 135V, and conservative substitutions thereof.

In some embodiments, the antigen-binding polypeptide construct has an H1L1 heterodimer comprising a combination of amino acid substitutions according to K-K cluster 7, wherein the HILL heterodimer comprises the following set of amino acid substitutions: H1 comprises no amino acid substitutions that promote preferential pairing, and L1 comprises 135W. In a further embodiment, the antigen-binding polypeptide construct has an H2L2 heterodimer comprising a combination of amino acid substitutions according to K-K cluster 7, wherein the H2L2 heterodimer comprises the following set of amino acid substitutions:H2 comprises 139W, and L2 comprises 116A or 116A_1335V. In some embodiments, the antigen-binding polypeptide construct comprises a combination of one H1L1 heterodimer with one H2L2 heterodimer.

In one embodiment, the antigen-binding polypeptide construct comprises a combination of amino acid substitutions according to K-K cluster 7 as set forth in one or the other of the designs in Table 10-B7.

### K-K Cluster 8:

In one embodiment, the antigen-binding polypeptide construct comprises a combination of amino acid substitutions according to K-K cluster 8 wherein: H1 comprises no amino acid substitutions that promote preferential pairing or comprises amino acid substitution at position 45; L1 comprises no amino acid substitutions that promote preferential pairing; H2 comprises amino acid substitution at position 45, and L2 comprises amino acid substitution at position 44.

In some embodiments, the antigen-binding polypeptide construct comprises a combination of amino acid substitutions according to K-K cluster 8 wherein the amino acid substitution in H1 is 45F, or conservative substitutions thereof; the amino acid substitution in H2 is 45P, 45A, or conservative substitutions thereof; the amino acid substitution in L2 is 44F or conservative substitutions thereof.

In some embodiments, the antigen-binding polypeptide construct has an H1L1 heterodimer comprising a combination of amino acid substitutions according to K-K cluster 8, wherein the H1L1 heterodimer comprises one of the following sets of amino acid substitutions: H1 comprises no amino acid substitutions that promote preferential pairing or comprises 45F, and L1 comprises no amino acid substitutions that promote preferential pairing. In further embodiments, the antigen-binding polypeptide construct has an H2L2 heterodimer comprising a combination of amino acid substitutions according to K-K cluster 8, wherein the H2L2 heterodimer comprises the following set of amino acid substitutions:H2 comprises 45A or 45P, and L2 comprises 44F. In some embodiments, the antigen-binding polypeptide construct comprises a combination of H1L1 and H2L2 heterodimers, where H1 and L1 comprise no amino acid substitutions that promote preferential pairing, H2 comprises 45A and L2 comprises 44F.

In one embodiment, the antigen-binding polypeptide construct comprises a combination of amino acid substitutions according to K-K cluster 8 as set forth in one or more of the designs in Table 10-B8.

### K-K Cluster 9:

In one embodiment, the antigen-binding polypeptide construct comprises a combination of amino acid substitutions according to K-K cluster 9 wherein: H1 comprises amino acid substitution at position 139; L1 comprises amino acid substitution at position 116; H2 comprises no amino acid substitutions that promote preferential pairing, and L2 comprises amino acid substitution at position 135.

In some embodiments, the antigen-binding polypeptide construct comprises a combination of amino acid substitutions according to K-K cluster 9 wherein the amino acid substitution in H1 is 139W or conservative substitutions thereof; the amino acid substitution in L1 is 116A or conservative substitutions thereof; and the amino acid substitution in L2 is 135W or conservative substitutions thereof.

In some embodiments, the antigen-binding polypeptide construct has an H1L1 heterodimer comprising a combination of amino acid substitutions according to K-K cluster 9, wherein the H1L1 heterodimer comprises the following set of amino acid substitutions: H1 comprises 139W, and L1 comprises 116A. In a further embodiment, the antigen-binding polypeptide construct has an H2L2 heterodimer comprising a combination of amino acid substitutions according to K-K cluster 9, wherein the H2L2 heterodimer comprises the following set of amino acid substitutions:H2 comprises no amino acid substitutions that promote preferential pairing, and L2 comprises 135W.

In one embodiment, the antigen-binding polypeptide construct comprises a combination of amino acid substitutions according to K-K cluster 9 as set forth in Table 10-B9.

### K-K Cluster 10:

In one embodiment, the antigen-binding polypeptide construct comprises a combination of amino acid substitutions according to K-K cluster 10 wherein: H1 comprises amino acid substitution at position 124; L1 comprises amino acid substitution at position 176; H2 comprises amino acid substitution at position 124, and L2 comprises amino acid substitution at position 176. In some embodiments, L1 and/or L2 further comprise amino acid substitution at position 133.

In one embodiment, the antigen-binding polypeptide construct comprises a combination of amino acid substitutions according to K-K cluster 10 wherein: H1 comprises amino acid substitution at position 124; L1 comprises amino acid substitution at positions 133_176; H2 comprises amino acid substitution at position 124, and L2 comprises amino acid substitution at positions 133_176.

In some embodiments, the antigen-binding polypeptide construct comprises a combination of amino acid substitutions according to K-K cluster 10 wherein the amino acid substitution in H1 is 124E or conservative substitutions thereof; the amino acid substitutions in L1 are selected from 133G, 176R or conservative substitutions thereof; the amino acid substitution in H2 is 124R, or conservative substitutions thereof; the amino acid substitutions in L2 are selected from 133G, 176D or conservative substitutions thereof.

In some embodiments, the antigen-binding polypeptide construct has an H1L1 heterodimer comprising a combination of amino acid substitutions according to K-K cluster 10, wherein the H1L1 heterodimer comprises the following set of amino acid substitutions: H1 comprises 124E, and L1 comprises 133G_176R. In further embodiments, the antigen-binding polypeptide construct has an H2L2 heterodimer comprising a combination of amino acid substitutions according to K-K cluster 10, wherein the H2L2 heterodimer comprises the following set of amino acid substitutions:H2 comprises 124R, and L2 comprises 133G_176D. In some embodiments, the antigen-binding polypeptide construct comprises a combination of these H1L1 and H2L2 heterodimers.

In one embodiment, the amino acid combinations of K-L cluster 9 comprise one or more secondary substitutions selected from Table F.

In one embodiment, the antigen-binding polypeptide construct comprises a combination of amino acid substitutions according to K-K cluster 10 as set forth in Table 10-B10.

### Preferential Pairing in LCCA Design Sets

One or more of H1, L1, H2, and L2 comprise amino acid modifications that promote preferential pairing of H1 with L1 as compared to L2 and of H2 with L2 as compared to L1. In general, in the absence of the amino acid modifications and any naturally occurring bias, a wild type immunoglobulin heavy chain sequence (H1), when co-expressed with two different wild type immunoglobulin light chain sequences (L1 and L2), will statistically pair equally with both light chains, resulting in an approximate 50:50 mixture of H1 paired with L1 (H1L1, correctly paired) and H1 paired with L2 (H1L2, mispaired). Likewise, if wild type H2 is co-expressed with wild type L1 and L2, the heavy chain will statistically pair equally with both light chains, resulting in an approximate 50:50 mixture of H2 paired with L1 (H2L1, mispaired) and H2 paired with L2 (H2L2, correctly paired). The term “preferential pairing” is used herein to describe the pairing specificity or pairing preference of an immunoglobulin heavy chain polypeptide sequence with one immunoglobulin light chain polypeptide sequence as compared to another immunoglobulin light chain polypeptide sequence. In this context, preferential pairing would occur between, for example, H1 and L1, if the amount of the HILL heterodimer is greater than the amount of the H1L2 heterodimer when H1 is co-expressed with both L1 and L2. Similarly, preferential pairing would occur between, for example, H2 and L2, if the amount of the H2L2 heterodimer is greater than the amount of the H2L1 heterodimer when H2 is co-expressed with both L1 and L2.

However, in some cases, there is an inherent pairing bias observed in the wild-type heavy and light chain polypeptide sequences obtained from the parent antibodies. This inherent pairing bias can be observed in the context of an LCCA design set in which wild-type parental H1 or H2 are co-expressed with wild-type parental L1 and L2, where one of the light chains preferentially pairs with the heavy chains of both parent antibodies. In one embodiment, preferential pairing occurs when the amino acid modifications in one or more of H1, L1, H2 and L2 promote preferential pairing that is greater than the preferential pairing that occurs in the corresponding wild type system.

The degree of preferential pairing, or design strength, is a measure of the ability of the amino acid modifications to promote preferential pairing. The degree of preferential pairing can be assessed as described elsewhere herein, and in the examples, and is based on the measurement of correctly paired heterodimers (i.e. H1L1 and H2L2) compared to mispaired heterodimers (i.e. H1L2 and H2L1). The degree of preferential pairing can be assessed in the context of LCCA design sets (H1L1L2, or H2L1L2) where one heavy chain is co-expressed with two unique light chains, or a Mab design set (H1L1H2L2) where the heavy and light chains of the parent antibody are co-expressed.

The following embodiments relate to the context of LCCA design sets. In all of the embodiments in this section, the term “about” means±5% of the specified ratio, and preferential pairing is compared with respect to wild-type, unless otherwise indicated. In one embodiment, one or more of H1, H2, L1 and L2 comprise amino acid modifications that promote preferential pairing of H1 with L1 as compared to L2 to form H1L1, or of H2 with L2 as compared to L1 to form H2L2, where the ratio of H1L1: H1L2 is at least about 40:60 and the ratio of H2L2:H2L1 is at least about 60:40. In one embodiment, one or more of H1, H2, L1 and L2 comprise amino acid modifications that promote preferential pairing of H1 with L1 as compared to L2 to form H1L1, or H2 with L2 as compared to L1 to form H2L2, where the ratio of H2L2:H2L1 is at least about 40:60 and the ratio of H1L1: H1L2 is at least about 60:40.

In one embodiment, one or more of H1, H2, L1 and L2 comprise amino acid modifications that promote preferential pairing of H1 with L1 as compared to L2 to form H1L1, or of H2 with L2 as compared to L1 to form H2L2, where the ratio of H1L1: H1L2 is at least about 40:60 and the ratio of H2L2:H2L1 is at least about 65:35. In one embodiment, one or more of H1, H2, L1 and L2 comprise amino acid modifications that promote preferential pairing of H1 with L1 as compared to L2 to form H1L1, or H2 with L2 as compared to L1 to form H2L2, where the ratio of H2L2:H2L1 is at least about 40:60 and the ratio of H1L1: H1L2 is at least about 65:35.

In one embodiment, one or more of H1, H2, L1 and L2 comprise amino acid modifications that promote preferential pairing of H1 with L1 as compared to L2 to form H1L1, or of H2 with L2 as compared to L1 to form H2L2, where the ratio of H1L1: H1L2 is at least about 40:60 and the ratio of H2L2:H2L1 is at least about 70:30. In one embodiment, one or more of H1, H2, L1 and L2 comprise amino acid modifications that promote preferential pairing of H1 with L1 as compared to L2 to form H1L1, or H2 with L2 as compared to L1 to form H2L2, where the ratio of H2L2:H2L1 is at least about 40:60 and the ratio of H1L1: H1L2 is at least about 70:30.

In one embodiment, one or more of H1, H2, L1 and L2 comprise amino acid modifications that promote preferential pairing of H1 with L1 as compared to L2 to form H1L1, or of H2 with L2 as compared to L1 to form H2L2, where the ratio of H1L1: H1L2 is at least about 40:60 and the ratio of H2L2:H2L1 is at least about 75:25. In one embodiment, one or more of H1, H2, L1 and L2 comprise amino acid modifications that promote preferential pairing of H1 with L1 as compared to L2 to form H1L1, or H2 with L2 as compared to L1 to form H2L2, where the ratio of H2L2:H2L1 is at least about 40:60 and the ratio of H1L1: H1L2 is at least about 75:25.

In one embodiment, one or more of H1, H2, L1 and L2 comprise amino acid modifications that promote preferential pairing of H1 with L1 as compared to L2 to form H1L1, or of H2 with L2 as compared to L1 to form H2L2, where the ratio of H1L1: H1L2 is at least about 40:60 and the ratio of H2L2:H2L1 is at least about 80:20. In one embodiment, one or more of H1, H2, L1 and L2 comprise amino acid modifications that promote preferential pairing of H1 with L1 as compared to L2 to form H1L1, or H2 with L2 as compared to L1 to form H2L2, where the ratio of H2L2:H2L1 is at least about 40:60 and the ratio of H1L1: H1L2 is at least about 80:20.

In one embodiment, one or more of H1, H2, L1 and L2 comprise amino acid modifications that promote preferential pairing of H1 with L1 as compared to L2 to form H1L1, or of H2 with L2 as compared to L1 to form H2L2, where the ratio of H1L1: H1L2 is at least about 40:60 and the ratio of H2L2:H2L1 is at least about 85:15. In one embodiment, one or more of H1, H2, L1 and L2 comprise amino acid modifications that promote preferential pairing of H1 with L1 as compared to L2 to form H1L1, or H2 with L2 as compared to L1 to form H2L2, where the ratio of H2L2:H2L1 is at least about 40:60 and the ratio of H1L1: H1L2 is at least about 85:15.

In one embodiment, one or more of H1, H2, L1 and L2 comprise amino acid modifications that promote preferential pairing of H1 with L1 as compared to L2 to form H1L1, or of H2 with L2 as compared to L1 to form H2L2, where the ratio of H1L1: H1L2 is at least about 40:60 and the ratio of H2L2:H2L1 is at least about 90:10. In one embodiment, one or more of H1, H2, L1 and L2 comprise amino acid modifications that promote preferential pairing of H1 with L1 as compared to L2 to form H1L1, or H2 with L2 as compared to L1 to form H2L2, where the ratio of H2L2:H2L1 is at least about 40:60 and the ratio of H1L1: H1L2 is at least about 90:10.

In one embodiment, one or more of H1, H2, L1 and L2 comprise amino acid modifications that promote preferential pairing of H1 with L1 as compared to L2 to form H1L1, or of H2 with L2 as compared to L1 to form H2L2, where the ratio of H1L1: H1L2 is at least about 40:60 and the ratio of H2L2:H2L1 is at least about 95:5. In one embodiment, one or more of H1, H2, L1 and L2 comprise amino acid modifications that promote preferential pairing of H1 with L1 as compared to L2 to form H1L1, or H2 with L2 as compared to L1 to form H2L2, where the ratio of H2L2:H2L1 is at least about 40:60 and the ratio of H1L1: H1L2 is at least about 95:5.

In one embodiment, one or more of H1, H2, L1 and L2 comprise amino acid modifications that promote preferential pairing of H1 with L1 as compared to L2 to form H1L1, or of H2 with L2 as compared to L1 to form H2L2, where the ratio of H1L1: H1L2 is at least about 40:60 and the ratio of H2L2:H2L1 is at least about 99:1. In one embodiment, one or more of H1, H2, L1 and L2 comprise amino acid modifications that promote preferential pairing of H1 with L1 as compared to L2 to form H1L1, or H2 with L2 as compared to L1 to form H2L2, where the ratio of H2L2:H2L1 is at least about 40:60 and the ratio of H1L1: H1L2 is at least about 99:1.

In other embodiments, one or more of H1, H2, L1 and L2 comprise amino acid modifications that promote preferential pairing of H1 with L1 as compared to L2 to form H1L1, or of H2 with L2 as compared to L1 to form H2L2, such that the amount of H1L1 or H2L2 is greater than about 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

In one embodiment, preferential pairing is measured by LCCA as described in the examples. The LCCA results are generally predictive of the results in the context of preferential pairing in Mab design sets (described below) in which H1, L1, H2, and L2 are co-expressed.

In one embodiment, one or more of H1, H2, L1 and L2 comprise amino acid modifications that promote preferential pairing of H1 with L1 as compared to L2 to form H1L1, or H2 with L2 as compared to L1 to form H2L2, such that the relative pairing of at least one of H1L1 or H2L2 is at least about 10% greater relative to wild-type, and the relative pairing of the other is within about 10% of wild-type or at least about 10% greater relative to wild-type.

In one embodiment, one or more of H1, H2, L1 and L2 comprise amino acid modifications that promote preferential pairing of H1 with L1 as compared to L2 to form H1L1, or H2 with L2 as compared to L1 to form H2L2, such that the relative pairing of at least one of H1L1 or H2L2 is at least about 20% greater relative to wild-type, and the relative pairing of the other is within about 10% of wild-type or at least about 10% greater relative to wild-type.

In one embodiment, one or more of H1, H2, L1 and L2 comprise amino acid modifications that promote preferential pairing of H1 with L1 as compared to L2 to form H1L1, or H2 with L2 as compared to L1 to form H2L2, such that the relative pairing of at least one of H1L1 or H2L2 is at least about 30% greater relative to wild-type, and the relative pairing of the other is within about 10% of wild-type or at least about 10% greater relative to wild-type.

### Preferential Pairing in Mab Design Sets

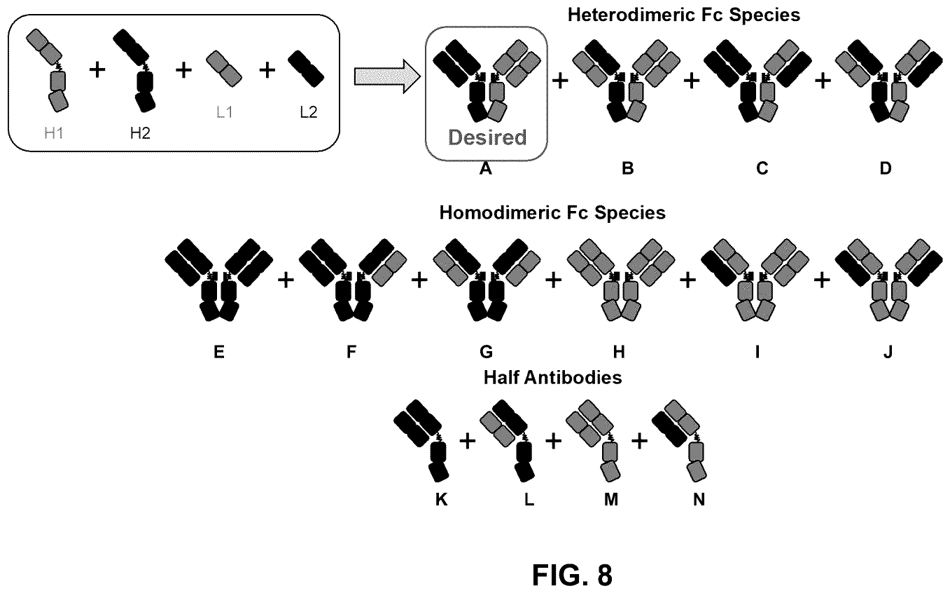

Preferential pairing can also be assessed in the context of Mab design sets where H1, L1, H2 and L2 are co-expressed and one or more of H1, L1, H2 and L2 comprise amino acid modifications that promote preferential pairing of H1 with L1 and of H2 with L2 to form a bispecific antigen-binding polypeptide construct that comprises a correctly paired first heterodimer (H1L1) and a correctly paired second heterodimer (H2L2). In this type of embodiment, as shown in FIG. 8, when the two distinct immunoglobulin heavy chain polypeptide sequences are co-expressed with two distinct immunoglobulin light chain polypeptide sequences, a number of potential products can result, fourteen of which are shown in FIG. 8, only one of which is the desired, or correctly paired, bispecific antibody H1L1H2L2 (antibody species A in FIG. 8). However, in the context of assessing correct pairing between heavy chains and light chain based on the Mab design sets, some of the additional products may also considered to exhibit correct pairing in the context of the Fab regions since they comprise correctly paired heterodimers at the Fab level (see for example antibody species E, H, K, and M in FIG. 8). In some embodiments, the Fc portion of the antigen-binding polypeptide construct comprises asymmetric amino acid modifications that promote formation of a heterodimeric Fc. In these embodiments, the number and amount of species E to J are expected to decrease.

As for the LCCA design sets, in the context of a Mab design set in which all four immunoglobulin polypeptide sequences H1, L1, H2, and L2 are co-expressed, in some cases there may be an inherent bias in pairing resulting in one of the light chains (either L1 or L2) preferentially pairing with both H1 and H2. Thus, when determining the strength of a Mab design in the context of a bispecific antigen-binding polypeptide construct, it may be necessary to assess the degree of pairing with the amino acid modifications of the Mab design compared to the amount of correct pairing observed in the corresponding wild-type parental system (H1, L1, H2, L2 polypeptide sequences without the amino acid modifications of the Mab design). Thus, in one embodiment, a Mab design is considered to show preferential pairing if the amount of correctly paired bispecific antigen-binding polypeptide construct is greater than the amount of correctly paired bispecific antibody obtained in the corresponding wild-type parental system. Alternatively, a Mab design is considered to show preferential pairing if the percentage of correctly paired bispecific antigen-binding polypeptide construct in the total expression product is greater than the percentage of correctly paired bispecific antibody obtained in the total expression product in the corresponding wild-type parental system. In one embodiment, the total expression product may comprise antibody species A to N in FIG. 8. In one embodiment, the total expression product may be only those antibody species that have two heavy chains and two light chains (antibody species A to J in FIG. 8). In the latter embodiment, preferential pairing is measured as a percentage of total bispecific antibody, excluding half-antibodies such as species K to N in FIG. 8).

In another embodiment, a Mab design is considered to show preferential pairing if the amount of correct pairing is increased in the heterodimer of the bispecific antigen-binding polypeptide construct that exhibits a high degree of mispairing in the corresponding wild-type parental system. In another embodiment, a Mab design is considered to show preferential pairing if the total amount of correct pairing between H1 and L1, and between H2 and L2, is greater than that observed in the corresponding wild-type parental system. For example, with reference to FIG. 8, species A, B, H, I, and M would be considered correctly paired with respect to H1L1, and species A, C, E, F, and K would be considered correctly paired with respect to H2L2. In this embodiment, preferential pairing is measured as a percentage of total pairing.

In one embodiment, the preferential pairing is measured by SMCA as described herein.

In some embodiments, a Mab design is considered to promote preferential pairing when the change in the amount of total correct pairing as measured by the sum of HILL and H2L2 pairing is greater than about 5%, 10%, 15%, 20%, 25%, 30%, 35%, 40%, or 45% compared to the pairing of corresponding H1, L1, H2 and L2 polypeptide chains without amino acid substitutions in the Fab region that promote preferential pairing.

In some embodiment, a Mab design is considered to promote preferential pairing when the change in the amount of total correct pairing, as measured by the amount of bispecific antibody produced as a percentage of species other than half-antibodies produced, is greater than about 5%, 10%, 20%, 30%, 40%, 50%, 60%, 70%, or 80%, compared to the pairing of corresponding H1, L1, H2 and L2 polypeptide chains without amino acid substitutions in the Fab region that promote preferential pairing.

In one embodiment, a Mab design is considered to promote preferential pairing when the change in the amount of total correct pairing, as measured by the amount of bispecific antibody produced as a percentage of all species produced, is greater than about 5%, 10%, 20%, 30%, 40%, 50%, 60%, 70%, or 80%, compared to the pairing of corresponding H1, L1, H2 and L2 polypeptide chains without amino acid substitutions in the Fab region that promote preferential pairing.

### Thermal Stability of Fab Regions

The amino acid modifications in one or more of the H1, L1, H2 and L2 polypeptide sequences promote preferential pairing of H1 with L1 as compared to L2 and of H2 with L2 as compared to L1, and minimally affect the thermal stability of each heterodimer of the antigen-binding polypeptide construct. The effect of the amino acid modifications on each heterodimer is determined by measuring the thermal stability of the Fab regions formed by H1 and L1, or by H2 and L2, and comparing it to the thermal stability of a Fab region formed by the corresponding wild type H1 and L1 polypeptide sequences (wt first Fab region) or by the corresponding wild type H2 and L2 polypeptide sequences (wt second Fab region). The terms “corresponding wild type H1 and L1 polypeptide sequences,” and “corresponding wild type H2 and L2 polypeptide sequences,” are meant to describe corresponding H1, L1, H2, and L2 polypeptide sequences that do not have the amino acid modifications that promote preferential pairing as described herein.

Thermal stability can be measured by a variety of methods known in the art and described herein, including differential scanning calorimetry (DSC), or differential scanning fluorimetry (DSF). The latter methods provide a measure of thermal stability in terms of “melting temperature” or Tm.

In the context of the following embodiments, the term “about” means±10% of the recited temperature. In one embodiment, the antigen-binding polypeptide construct comprises a first Fab region that has a Tm within about 20° C. of the Tm of the corresponding wt first Fab region. In one embodiment, the antigen-binding polypeptide construct comprises a first Fab region that has a Tm within about 15° C. of the Tm of the corresponding wt first Fab region. In one embodiment, the antigen-binding polypeptide construct comprises a first Fab region that has a Tm within about 10° C. of the Tm of the corresponding wt first Fab region. In one embodiment, the antigen-binding polypeptide construct comprises a first Fab region that has a Tm within about 9° C. of the Tm of the corresponding wt first Fab region. In one embodiment, the antigen-binding polypeptide construct comprises a first Fab region that has a Tm within about 8° C. of the Tm of the corresponding wt first Fab region. In one embodiment, the antigen-binding polypeptide construct comprises a first Fab region that has a Tm within about 7° C. of the Tm of the corresponding wt first Fab region. In one embodiment, the antigen-binding polypeptide construct comprises a first Fab region that has a Tm within about 6° C. of the Tm of the corresponding wt first Fab region. In one embodiment, the antigen-binding polypeptide construct comprises a first Fab region that has a Tm within about 5° C. of the Tm of the corresponding wt first Fab region. In one embodiment, the antigen-binding polypeptide construct comprises a first Fab region that has a Tm within about 4° C. of the Tm of the corresponding wt first Fab region. In one embodiment, the antigen-binding polypeptide construct comprises a first Fab region that has a Tm within about 3° C. of the Tm of the corresponding wt first Fab region. In one embodiment, the antigen-binding polypeptide construct comprises a first Fab region that has a Tm within about 2° C. of the Tm of the corresponding wt first Fab region. In one embodiment, the antigen-binding polypeptide construct comprises a first Fab region that has a Tm within about 1° C. of the Tm of the corresponding wt first Fab region. In one embodiment, the antigen-binding polypeptide construct comprises a first Fab region that has a Tm that is about the same as that of the corresponding wt first Fab region.

In one embodiment, the antigen-binding polypeptide construct comprises a second Fab region that has a Tm within about 20° C. of the Tm of the corresponding wt second Fab region. In one embodiment, the antigen-binding polypeptide construct comprises a second Fab region that has a Tm within about 15° C. of the Tm of the corresponding wt second Fab region. In one embodiment, the antigen-binding polypeptide construct comprises a second Fab region that has a Tm within about 10° C. of the Tm of the corresponding wt second Fab region. In one embodiment, the antigen-binding polypeptide construct comprises a second Fab region that has a Tm within about 9° C. of the Tm of the corresponding wt second Fab region. In one embodiment, the antigen-binding polypeptide construct comprises a second Fab region that has a Tm within about 8° C. of the Tm of the corresponding wt second Fab region. In one embodiment, the antigen-binding polypeptide construct comprises a second Fab region that has a Tm within about 7° C. of the Tm of the corresponding wt second Fab region. In one embodiment, the antigen-binding polypeptide construct comprises a second Fab region that has a Tm within about 6° C. of the Tm of the corresponding wt second Fab region. In one embodiment, the antigen-binding polypeptide construct comprises a second Fab region that has a Tm within about 5° C. of the Tm of the corresponding wt second Fab region. In one embodiment, the antigen-binding polypeptide construct comprises a second Fab region that has a Tm within about 4° C. of the Tm of the corresponding wt second Fab region. In one embodiment, the antigen-binding polypeptide construct comprises a second Fab region that has a Tm within about 3° C. of the Tm of the corresponding wt second Fab region. In one embodiment, the antigen-binding polypeptide construct comprises a second Fab region that has a Tm within about 2° C. of the Tm of the corresponding wt second Fab region. In one embodiment, the antigen-binding polypeptide construct comprises a second Fab region that has a Tm within about 1° C. of the Tm of the corresponding wt second Fab region. In one embodiment, the antigen-binding polypeptide construct comprises a second Fab region that has a Tm that is about the same as that of the corresponding wt second Fab region.

In one embodiment, the antigen-binding polypeptide construct comprises a first heterodimer and a second heterodimer wherein the melting temperature (Tm) of the first Fab region is within about 0, 1, 2, 3, 4, 5, 6, 7, 8, 9, 10, or 20° C. of the Tm of a Fab region formed by the corresponding wild type H1 and L1 polypeptide sequences for the first antigen (wt first Fab region), and/or the melting temperature (Tm) of the second Fab region is within about 0, 1, 2, 3, 4, 5, 6, 7, 8, 9, 10, or 20° C. of the Tm of a Fab region formed by the corresponding wild type H2 and L2 polypeptide sequences for the second antigen (wt second Fab region).

Furthermore, in some embodiments, the Tm of the first or second Fab region is greater than that of the corresponding wt first Fab or corresponding second wt second Fab. Thus, in one embodiment, the Tm of the first or second Fab region is increased by about 0.1, 0.2, 0.3, 0.4, 0.5, 0.6, 0.7, 0.8. 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.1, 3.2, 3.3, 3.4, 3.5, 3.6, 3.7, 3.8, 3.9, 4.0, 4.5, 5.0° C. or more compared to the corresponding wt first Fab region or corresponding wt second Fab region.

In one embodiment, the antigen-binding polypeptide construct comprises amino acid substitutions corresponding to K-L design numbers 2979, 3018, 3041, 3102, 3898, and/or 3947. In one embodiment, the antigen-binding polypeptide construct comprises amino acid substitutions corresponding to K-L design numbers 3025, 3109, 3113, 3878, 3890, 3910, 3931, 3954, 3967, 4010, and/or 4040.

### Ability of Fab Regions to Bind Antigen

The amino acid modifications in one or more of the H1, L1, H2 and L2 polypeptide sequences promote preferential pairing of H1 with L1 as compared to L2 and of H2 with L2 as compared to L1, and minimally affect the ability of each heterodimer of the antigen-binding polypeptide construct to bind to its antigen. The effect of the amino acid modifications on each heterodimer is determined by measuring the ability of the Fab regions formed by H1 and L1, or by H2 and L2, to bind to their respective antigens, and comparing it to the ability of the corresponding wt first Fab region, or corresponding wt second Fab region to bind to their respective antigens.

The ability of the Fab regions to bind to their respective antigens can be measured by a number of methods known in the art, some of which are described elsewhere herein. For example surface plasmon resonance (SPR), or whole cell binding assays may be used to assess the ability of the first Fab region to bind a first antigen and of the second Fab region to bind a second antigen. The latter two methods measure the ability of the Fab regions to bind to their respective antigens by determining the affinity of the Fab region for its antigen.

In one embodiment, the affinity of the first Fab region for the first antigen is within about 100-fold of the affinity of the wt first Fab region for the first antigen. In one embodiment, the affinity of the first Fab region for the first antigen is within about 50-fold of the affinity of the wt first Fab region for the first antigen. In one embodiment, the affinity of the first Fab region for the first antigen is within about 40-fold of the affinity of the wt first Fab region for the first antigen. In one embodiment, the affinity of the first Fab region for the first antigen is within about 30-fold of the affinity of the wt first Fab region for the first antigen. In one embodiment, the affinity of the first Fab region for the first antigen is within about 20-fold of the affinity of the wt first Fab region for the first antigen. In one embodiment, the affinity of the first Fab region for the first antigen is within about 10-fold of the affinity of the wt first Fab region for the first antigen. In one embodiment, the affinity of the first Fab region for the first antigen is within about 9-fold of the affinity of the wt first Fab region for the first antigen. In one embodiment, the affinity of the first Fab region for the first antigen is within about 8-fold of the affinity of the wt first Fab region for the first antigen. In one embodiment, the affinity of the first Fab region for the first antigen is within about 7-fold of the affinity of the wt first Fab region for the first antigen. In one embodiment, the affinity of the first Fab region for the first antigen is within about 6-fold of the affinity of the wt first Fab region for the first antigen. In one embodiment, the affinity of the first Fab region for the first antigen is within about 5-fold of the affinity of the wt first Fab region for the first antigen. In one embodiment, the affinity of the first Fab region for the first antigen is within 4-fold of the affinity of the wt first Fab region for the first antigen. In one embodiment, the affinity of the first Fab region for the first antigen is within about 3-fold of the affinity of the wt first Fab region for the first antigen. In one embodiment, the affinity of the first Fab region for the first antigen is within about 2-fold of the affinity of the wt first Fab region for the first antigen. In one embodiment, the affinity of the first Fab region for the first antigen is about the same as the affinity of the wt first Fab region for the first antigen.

In one embodiment, the affinity of the second Fab region for the second antigen is within about 100-fold of the affinity of the wt second Fab region for the second antigen. In one embodiment, the affinity of the second Fab region for the second antigen is within about 50-fold of the affinity of the wt second Fab region for the second antigen. In one embodiment, the affinity of the second Fab region for the second antigen is within about 40-fold of the affinity of the wt second Fab region for the second antigen. In one embodiment, the affinity of the second Fab region for the second antigen is within about 30-fold of the affinity of the wt second Fab region for the second antigen. In one embodiment, the affinity of the second Fab region for the second antigen is within about 20-fold of the affinity of the wt second Fab region for the second antigen. In one embodiment, the affinity of the second Fab region for the second antigen is within about 10-fold of the affinity of the wt second Fab region for the second antigen. In one embodiment, the affinity of the second Fab region for the second antigen is within about 9-fold of the affinity of the wt second Fab region for the second antigen. In one embodiment, the affinity of the second Fab region for the second antigen is within about 8-fold of the affinity of the wt second Fab region for the second antigen. In one embodiment, the affinity of the second Fab region for the second antigen is within about 7-fold of the affinity of the wt second Fab region for the second antigen. In one embodiment, the affinity of the second Fab region for the second antigen is within about 6-fold of the affinity of the wt second Fab region for the second antigen. In one embodiment, the affinity of the second Fab region for the second antigen is within about 5-fold of the affinity of the wt second Fab region for the second antigen. In one embodiment, the affinity of the second Fab region for the second antigen is within 4-fold of the affinity of the wt second Fab region for the second antigen. In one embodiment, the affinity of the second Fab region for the second antigen is within about 3-fold of the affinity of the wt second Fab region for the second antigen. In one embodiment, the affinity of the second Fab region for the second antigen is within about 2-fold of the affinity of the wt second Fab region for the second antigen. In one embodiment, the affinity of the second Fab region for the second antigen is about the same as the affinity of the wt second Fab region for the second antigen.

### Transferability of Amino Acid Modifications or Design Sets

The amino acid modifications or design sets described herein can be used to prepare a bispecific antigen-binding polypeptide construct in which the immunoglobulin heavy chain polypeptide sequences and immunoglobulin light chain polypeptides of each heterodimer can be obtained from one or more parent antibodies, where at least one parent antibody comprises a kappa light chain, and at least one other parent antibody comprises a lambda light chain. Based on the following discussion, the Mab design sets can be applied to the majority of such bispecific antigen-binding polypeptide constructs.

The VH: VL and CH1: CL interface residues in the interface between immunoglobulin heavy and light chains are relatively well conserved (Padlan et al., 1986, Mol. Immunol. 23 (9): 951-960). This sequence conservation, a result of evolutionary constraints, increases the likelihood that functionally active antibody binding domains will be formed during combinatorial pairing of light and heavy chains. As a result of this sequence conservation, it follows that the Mab design sets described herein and based on modeling of the structures of the D3H44 kappa Fab and CAT-2200 lambda Fab, which drive preferential pairing, can be transferred to the kappa Fabs and lambda Fabs of other parent antibodies to drive preferential pairing, since this region displays high sequence conservation across antibodies. Further, when sequence differences do occur, these usually lie distal to the CH1: CL interface. This is particularly the case for the CH1 and CL domains. In an embodiment, the antigen-binding polypeptide constructs described herein comprise heterodimers where the kappa Fab comprises one or more amino acid modifications in CL and/or CH1 domains that promote preferential pairing. In an embodiment, the antigen-binding polypeptide constructs described herein comprise heterodimers where the lambda Fab comprises one or more amino acid modifications in CL and/or CH1 domains that promote preferential pairing.

There is, however, some sequence variability at the antigen-binding site with respect to CDR (complementarity-determining regions) loop residues (and length), particularly for CDR-H3. Thus, in one embodiment, the antigen-binding polypeptide constructs described herein comprise heterodimers where the kappa Fab comprises one or more amino acid modifications in the VH and/or VL domains that lie distal to the CDR loops when the amino acid sequence of the antigen-binding site is significantly different from that of the D3H44 antibody. In another embodiment, the antigen-binding polypeptide constructs described herein comprise heterodimers where the lambda Fab comprises one or more amino acid modifications in the VH and/or VL domains that promote preferential pairing and that lie distal to the CDR loops when the amino acid sequence of the antigen-binding site is significantly different from that of the CAT-2200 antibody. In another embodiment, the antigen-binding polypeptide constructs described herein comprise heterodimers where the kappa Fab comprises one or more amino acid modifications that promote preferential pairing in the VH and/or VL domains that lie proximal or distal to the CDR loops, when the amino acid sequence of the antigen-binding site is substantially similar to that of the D3H44 antibody. In another embodiment, the antigen-binding polypeptide constructs described herein comprise heterodimers where the lambda Fab comprises one or more amino acid modifications in the VH and/or VL domains that promote preferential pairing and that lie proximal or distal to the CDR loops, when the amino acid sequence of the antigen-binding site is substantially similar to that of the CAT-2200 antibody. In an embodiment, the antigen-binding polypeptide constructs described herein comprise heterodimers where the kappa Fab comprises one or more amino acid modifications in CL and/or CH1 domains as well as modifications in the VH and/or VL domains that promote preferential pairing. In an embodiment, the antigen-binding polypeptide constructs described herein comprise heterodimers where the lambda Fab comprises one or more amino acid modifications in CL and/or CH1 domains as well as modifications in the VH and/or VL domains that promote preferential pairing.

In one embodiment, the amino acid modifications in one or more of H1, L1, H2, and L2 of the antigen-binding polypeptide construct can promote preferential pairing in an antigen-binding polypeptide construct where the kappa Fab of one parent antibody is a human or humanized IgG1/K. Non-limiting examples of such parent antibodies include Ofatumumab (human) or Trastuzumab, or Bevacizumab (humanized). In one embodiment, the amino acid modifications in one or more of H1, L1, H2, and L2 of the antigen-binding polypeptide construct can promote preferential pairing in an antigen-binding polypeptide construct where the lambda Fab of one parent antibody is a human or humanized IgG1/lambda. Non-limiting examples of such human antibodies include Briakinumab or Sifalimumab, while an example of a humanized antibody is Brontictuzumab.

In another embodiment, the amino acid modifications described herein are transferable to the immunoglobulin heavy and light chains of antibodies utilizing commonly used VH and VL subgroups.

In one embodiment, the amino acid modifications described herein are transferable to the immunoglobulin heavy and light chains of antibodies having a framework close to germline. Examples of such antibodies include Obinutuzumab.

In one embodiment, the amino acid modifications described herein are transferable to the immunoglobulin heavy and light chains of antibodies having a VH: VL interdomain angle close to the average observed for heavy and light chain pairs. An example of this type of antibody includes, but is not limited to Pertuzumab. In another embodiment, the amino acid modifications described herein are transferable to the immunoglobulin heavy and light chains of antibodies having canonical CL and CH1 domains. Suitable examples of such antibodies include, but are not limited to Trastuzumab.

The Examples, Figures, and Tables demonstrate that the amino acid modifications (e.g., within one or more Fab fragments comprising a variable region and a constant region) that promote preferential pairing are transferable to other immunoglobulin heavy and light chains, resulting in similar patterns of preferential pairing of one immunoglobulin heavy chain with one of the two immunoglobulin light chains.

### Scaffolds

The heterodimers of the antigen-binding polypeptide construct can be linked to a scaffold. A scaffold may be a peptide, polypeptide, polymer, nanoparticle or other chemical entity. The heterodimers of the antigen-binding polypeptide construct may be linked to either the N- or C-terminus of the scaffold, where the scaffold is a polypeptide. In one embodiment, the scaffold is an albumin polypeptide.

In another embodiment, the scaffold is an immunoglobulin Fc (Fc), or portion thereof. In some embodiments, the Fc comprises at least one or two CH3 domain sequences. In some embodiments, the Fc further comprises at least one or two CH2 domain sequences. In some embodiments the antigen-binding polypeptide construct comprises an Fc that is coupled, with or without one or more linkers, to the first heterodimer and/or the second heterodimer. In some embodiments, the Fc is a human Fc. In some embodiments, the Fc is a human IgG or IgG1 Fc. In some embodiments, the Fc is a heterodimeric Fc. In some embodiments, an Fc is a single polypeptide. In some embodiments, an Fc is multiple peptides, e.g., two polypeptides.

In some embodiments, the Fc comprises one or more amino acid modifications in at least one of the CH3 domain sequences. Amino acid modifications can be made to the immunoglobulin Fc in order to drive preferential pairing between heterodimeric CH3 domain sequences relative to homodimeric CH3 domain sequences. Such amino acid modifications are known in the art and include, for example, those described, in US Patent Publication No. 2012/0149876. Alternate strategies for driving preferential pairing between heterodimeric CH3 domain sequences relative to homodimeric CH3 sequences include, for example, “knobs into holes”, charged residues with ionic interactions, and strand-exchange engineered domain (SEED) technologies can also be employed. The latter strategies have been described in the art and are reviewed in Klein et al, supra. Further discussion of Fc domains follows below.

In some aspects, Fc is an Fc described in patent applications PCT/CA2011/001238, filed Nov. 4, 2011 or PCT/CA2012/050780, filed Nov. 2, 2012, the entire disclosure of each of which is hereby incorporated by reference in its entirety for all purposes.

In some aspects, the antigen-binding polypeptide construct described herein comprises a heterodimeric Fc comprising a modified CH3 domain that has been asymmetrically modified. The heterodimeric Fc can comprise two heavy chain constant domain polypeptides: a first heavy chain polypeptide and a second heavy chain polypeptide, which can be used interchangeably provided that Fc comprises one first heavy chain polypeptide and one second heavy chain polypeptide. Generally, the first heavy chain polypeptide comprises a first CH3 sequence and the second heavy chain polypeptide comprises a second CH3 sequence.

Two CH3 sequences that comprise one or more amino acid modifications introduced in an asymmetric fashion generally results in a heterodimeric Fc, rather than a homodimer, when the two CH3 sequences dimerize. As used herein, “asymmetric amino acid modifications” refers to any modification where an amino acid at a specific position on a first CH3 sequence is different from the amino acid on a second CH3 sequence at the same position, and the first and second CH3 sequence preferentially pair to form a heterodimer, rather than a homodimer. This heterodimerization can be a result of modification of only one of the two amino acids at the same respective amino acid position on each sequence; or modification of both amino acids on each sequence at the same respective position on each of the first and second CH3 sequences. The first and second CH3 sequence of a heterodimeric Fc can comprise one or more than one asymmetric amino acid modification.

Table X provides the amino acid sequence of the human IgG1 Fc sequence, corresponding to amino acids 231 to 447 of the full-length human IgG1 heavy chain. The CH3 sequence comprises amino acid 341-447 of the full-length human IgG1 heavy chain.

Typically an Fc can include two contiguous heavy chain sequences (A and B) that are capable of dimerizing. In some aspects, one or both sequences of an Fc include one or more mutations or modifications at the following locations: L351, F405, Y407, T366, K392, T394, T350, S400, and/or N390, using EU numbering. In some aspects, an Fc includes a mutant sequence shown in Table X. In some aspects, an Fc includes the mutations of Variant 1 A-B. In some aspects, an Fc includes the mutations of Variant 2 A-B. In some aspects, an Fc includes the mutations of Variant 3 A-B. In some aspects, an Fc includes the mutations of Variant 4 A-B. In some aspects, an Fc includes the mutations of Variant 5 A-B.

TABLE X

| Human IgG1 | APELLGGPSVFLFPPKPKDTLMISRTPEVTCVVVDVSH | |
|---|---|---|
| Fc sequence | EDPEVKFNWYVDGVEVHNAKTKPREEQYNSTYRVVS | |
| 231-447 (EU- | VLTVLHQDWLNGKEYKCKVSNKALPAPIEKTISKAKG | |
| numbering), | QPREPQVYTLPPSRDELTKNQVSLTCLVKGFYPSDIAV | |
| SEQ ID NO: 11 | EWESNGQPENNYKTTPPVLDSDGSFFLYSKLTVDKSR | |
| | WQQGNVFSCSVMHEALHNHYTQKSLSLSPGK | |
| Variant IgG1 | | |
| Fc sequence | | |
| (231-447) | Chain | Mutations |
| 1 | A | L351Y_F405A_Y407V |
| 1 | B | T366L_K392M_T394W |
| 2 | A | L351Y_F405A_Y407V |
| 2 | B | T366L_K392L_T394W |
| 3 | A | T350V_L351Y_F405A_Y407V |
| 3 | B | T350V_T366L_K392L_T394W |
| 4 | A | T350V_L351Y_F405A_Y407V |
| 4 | B | T350V_T366L_K392M_T394W |
| 5 | A | T350V_L351Y_S400E_F405A_Y407V |
| 5 | B | T350V_T366L_N390R_K392M_T394W |

In some embodiments, the Fc can comprise one or more amino acid modifications in at least one of the CH2 domain sequences. A number of mutations in the heavy chain sequence of the Fc are known in the art for selectively altering the affinity of the antibody Fc for different Fcgamma receptors. In some embodiments, the Fc comprises one or more modifications to alter binding of Fc-gamma receptors to the antigen-binding polypeptide construct.

The CH2 domain corresponds to amino acids 231-340 of the sequence shown in Table X. Exemplary, non-limiting amino acid modifications that alter the ability of the Fc of the antigen-binding polypeptide construct to bind to Fc-gamma receptors are listed below:
S298A/E333A/K334A, S298A/E333A/K334A/K326A (Lu Y, Vernes J M, Chiang N, et al. J Immunol Methods. 2011 Feb. 28; 365 (1-2): 132-41);
F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67 (18): 8882-90; Nordstrom J L, Gorlatov S, Zhang W, et al. Breast Cancer Res. 2011 Nov. 30; 13 (6): R123); F243L (Stewart R, Thom G, Levens M, et al. Protein Eng Des Sel. 2011 September; 24 (9): 671-8), S298A/E333A/K334A (Shields RL, Namenuk A K, Hong K, et al. J Biol Chem. 2001 Mar. 2; 276 (9): 6591-604);
S239D/1332E/A330L, S239D/1332E (Lazar G A, Dang W, Karki S, et al. Proc Natl Acad Sci USA. 2006 Mar. 14; 103 (11): 4005-10); S239D/S267E, S267E/L328F (Chu S Y, Vostiar I, Karki S, et al. Mol Immunol. 2008 September; 45 (15): 3926-33);
S239D/D265S/S298A/1332E, S239E/S298A/K326A/A327H, G237F/S298A/A330L/I332E, S239D/1332E/S298A, S239D/K326E/A330L/1332E/S298A, G236A/S239D/D270L/1332_E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) describes additional modifications to the Fc that affect binding of the Fc to Fc-gamma receptors on page 283.

### Additional Modifications to Improve Effector Function.

In some embodiments the Fc of the antigen-binding polypeptide construct described herein can be modified to improve its effector function. Such modifications are known in the art and include afucosylation, or engineering of the affinity of the Fc portion of antibodies towards an activating receptor, mainly FCGR3a for ADCC, and towards C1q for CDC. The following Table Y summarizes various designs reported in the literature for effector function engineering.

TABLE Y

| Reference | Mutations | Effect |
|---|---|---|
| Lu, 2011, | Afucosylated | Increased |
| Ferrara 2011, | | ADCC |
| Mizushima 2011 | | |
| Lu, 2011 | S298A/E333A/K334A | Increased |
| | | ADCC |
| Lu, 2011 | S298A/E333A/K334A/K326A | Increased |
| | | ADCC |
| Stavenhagen, | F243L/R292P/Y300L/V305I/P396L | Increased |
| 2007 | | ADCC |
| Nordstrom, 2011 | F243L/R292P/Y300L/L235V/P396L | Increased |
| | | ADCC |
| Stewart, 2011 | F243L | Increased |
| | | ADCC |
| Shields, 2001 | S298A/E333A/K334A | Increased |
| | | ADCC |
| Lazar, 2006 | S239D/I332E/A330L | Increased |
| | | ADCC |
| Lazar, 2006 | S239D/I332E | Increased |
| | | ADCC |
| Bowles, 2006 | AME-D, not specified mutations | Increased |
| | | ADCC |
| Heider, 2011 | 37.1, mutations not disclosed | Increased |
| | | ADCC |
| Moore, 2010 | S267E/H268F/S324T | Increased |
| | | CDC |

Thus, in one embodiment, an antigen-binding polypeptide construct described herein can include a dimeric Fc that comprises one or more amino acid modifications as noted in the above table that confer improved effector function. In another embodiment, the antigen-binding polypeptide construct can be afucosylated to improve effector function.

### FcRn Binding and PK Parameters

As is known in the art, binding to FcRn recycles endocytosed antibody from the endosome back to the bloodstream (Raghavan et al., 1996, Annu Rev Cell Dev Biol 12:181-220; Ghetie et al., 2000, Annu Rev Immunol 18:739-766). This process, coupled with preclusion of kidney filtration due to the large size of the full-length molecule, results in favorable antibody serum half-lives ranging from one to three weeks. Binding of Fc to FcRn also plays a key role in antibody transport. Thus, in one embodiment, the Fc comprises one or more amino acid modifications that alter or promote the ability of the Fc to bind FcRn.

### Linkers

The constructs described herein can include one or more heterodimers described herein operatively coupled to an Fc described herein. In some aspects, Fc is coupled to the one or more heterodimers with or without one or more linkers. In some aspects, Fc is directly coupled to the one or more heterodimers. In some aspects, Fc is coupled to the one or more heterodimers by one or more linkers. In some aspects, Fc is coupled to the heavy chain of each heterodimer by a linker.

In some aspects, the one or more linkers are one or more polypeptide linkers. In some aspects, the one or more linkers comprise one or more antibody hinge regions. In some aspects, the one or more linkers comprise one or more IgG1 hinge regions.

### Additional Optional Modifications

In one embodiment, the immunoglobulin heavy and light chains of the antigen-binding polypeptide construct described herein can be further modified (i.e., by the covalent attachment of various types of molecules) such that covalent attachment does not interfere with the preferential pairing between heavy chain and light chains or affect the ability of the heterodimer to bind to its antigen, or affect its stability. Such modification include, for example, but not by way of limitation, glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, metabolic synthesis of tunicamycin, etc.

In another embodiment, the immunoglobulin heavy and light chains of the antigen-binding polypeptide construct described herein can be conjugated (directly or indirectly) to a therapeutic agent or drug moiety that modifies a given biological response. In certain embodiments an antigen-binding polypeptide construct is conjugated to a drug, e.g., a toxin, a chemotherapeutic agent, an immune modulator, or a radioisotope. Several methods of preparing ADCs (antibody-drug conjugates or antigen-binding polypeptide construct drug conjugates) are known in the art and are described in U.S. Pat. No. 8,624,003 (pot method), 8,163,888 (one-step), and 5,208,020 (two-step method) for example. In some embodiments, the drug is selected from a maytansine, auristatin, calicheamicin, or derivative thereof. In other embodiments, the drug is a maytansine selected from DM1 and DM4.

In some embodiments the antigen-binding polypeptide construct is conjugated to a cytotoxic agent. The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32, and Lu177), chemotherapeutic agents, and toxins such as small molecule toxins or enzymatically active toxins of bacterial, fungal, plant or animal origin, including fragments and/or variants thereof.

Therapeutic agents or drug moieties are not to be construed as limited to classical chemical therapeutic agents. For example, the drug moiety can be a protein or polypeptide possessing a desired biological activity. Such proteins can include, for example, a toxin such as abrin, ricin A, Onconase (or another cytotoxic RNase), pseudomonas exotoxin, cholera toxin, or diphtheria toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, in an alternate embodiment, the antigen-binding polypeptide construct can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483; Peterson et al., 1999, Bioconjug. Chem. 10:553; and Zimmerman et al., 1999, Nucl. Med. Biol. 26:943.

In some embodiments, the immunoglobulin heavy and light chains of the antigen-binding polypeptide construct are expressed as fusion proteins comprising a tag to facilitate purification and/or testing etc. As referred to herein, a “tag” is any added series of amino acids which are provided in a protein at either the C-terminus, the N-terminus, or internally that contributes to the identification or purification of the protein. Suitable tags include but are not limited to tags known to those skilled in the art to be useful in purification and/or testing such as albumin binding domain (ABD), His tag, FLAG tag, glutathione-s-transferase, hemagglutinin (HA) and maltose binding protein. Such tagged proteins can also be engineered to comprise a cleavage site, such as a thrombin, enterokinase or factor X cleavage site, for ease of removal of the tag before, during or after purification.

### Methods of Preparing Antigen-Binding Polypeptide Constructs

As described above, the antigen-binding polypeptide constructs described herein can comprise a first heterodimer and a second heterodimer, the first heterodimer comprising an immunoglobulin heavy chain or fragment thereof having at least a VH and CH1 domain, and an immunoglobulin lambda light chain having a VL domain and a CL domain, and the second heterodimer comprising an immunoglobulin heavy chain or fragment thereof having at least a VH and CH1 domain, and an immunoglobulin kappa light chain having a VL domain and a CL domain. The immunoglobulin polypeptide sequences are engineered to incorporate amino acid modifications that promote preferential pairing as described herein. Accordingly, in the case of a bispecific antigen-binding polypeptide construct, there are typically four distinct polypeptide sequences, two immunoglobulin heavy chain polypeptide sequences or fragments thereof and two immunoglobulin light chain polypeptide sequences, that make up the antigen-binding polypeptide construct. The immunoglobulin heavy chain polypeptide sequences and immunoglobulin light chain polypeptide sequences of the antigen-binding polypeptide construct can readily be prepared using recombinant DNA technology known in the art. Standard techniques such as, for example, those described in Sambrook and Russell, Molecular Cloning: A Laboratory Manual (Cold Spring Harbor Laboratory Press, Cold Spring Harbor, N.Y., 3rd ed., 2001); Sambrook et al., Molecular Cloning: A Laboratory Manual (Cold Spring Harbor Laboratory Press, Cold Spring Harbor, N.Y., 2nd ed., 1989); Short Protocols in Molecular Biology (Ausubel et al., John Wiley and Sons, New York, 4th ed., 1999); and Glick and Pasternak, Molecular Biotechnology: Principles and Applications of Recombinant DNA (ASM Press, Washington, D.C., 2nd ed., 1998) can be used for recombinant nucleic acid methods, nucleic acid synthesis, cell culture, transgene incorporation, and recombinant protein expression.

The polynucleotide and amino acid sequences of the immunoglobulin heavy and light chains of the parent antibodies that make up the antigen-binding polypeptide construct are either known in the art or can be readily determined using nucleic acid and/or protein sequencing methods.

Accordingly, also provided are polynucleotides or a set of polynucleotides encoding the immunoglobulin heavy and light chains of the antigen-binding polypeptide construct. Such polynucleotides include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The polynucleotides include full-length genes or cDNA molecules as well as a combination of fragments thereof.

The polynucleotides encoding the engineered immunoglobulin heavy and light chain polypeptides described herein can be prepared by site specific mutagenesis of nucleotides in the DNA encoding the polypeptide, using cassette or PCR mutagenesis or other techniques well known in the art, to produce DNA encoding the engineered immunoglobulin heavy and light chain polypeptides, and thereafter expressing the recombinant DNA in cell culture as outlined herein. However, polynucleotides encoding the engineered immunoglobulin heavy and light chain polypeptides may also be prepared by in vitro gene synthesis using established techniques.

As will be appreciated by those in the art, due to the degeneracy of the genetic code, an extremely large number of polynucleotides may be made, all of which encode the engineered immunoglobulin heavy and light chain polypeptides described herein. Thus, having identified a particular amino acid sequence, those skilled in the art could make any number of different polynucleotides, by simply modifying the sequence of one or more codons in a way which does not change the amino acid sequence of the encoded protein.

Also provided are expression systems and constructs in the form of plasmids, expression vectors, transcription or expression cassettes which comprise at least one polynucleotide as above. Also provided are host cells comprising such expression systems or constructs.

Typically, expression vectors used in the host cells will contain sequences for plasmid maintenance and for cloning and expression of exogenous nucleotide sequences. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the polynucleotide encoding the polypeptide to be expressed, and a selectable marker element. The vector can be multicistronic i.e. expressing two or more of the polynucleotides encoding the immunoglobulin heavy and light chains of the antigen-binding polypeptide construct, or the antigen-binding polypeptide construct can be expressed by a set of vectors, each vector expressing one or more of the polynucleotides. The antigen-binding polypeptide construct can also be expressed using a set of vectors comprising a combination of multicistronic vectors and vectors that comprise a single polynucleotide encoding one of the immunoglobulin heavy and light chains.

In some embodiments, the vector may contain a “tag”-encoding sequence, i.e., an oligonucleotide molecule located at the 5′ or 3′ end of the polypeptide coding sequence; the oligonucleotide sequence encodes polyHis (such as hexaHis), or another “tag” such as FLAG, HA (hemaglutinin influenza virus), or myc, for which commercially available antibodies exist. This tag is typically fused to the polypeptide upon expression of the polypeptide, and can serve as a means for affinity purification or detection of the polypeptide from the host cell. Affinity purification can be accomplished, for example, by column chromatography using antibodies against the tag as an affinity matrix. Optionally, the tag can subsequently be removed from the purified polypeptide by various means such as peptidase cleavage.

Vectors typically contain a promoter that is recognized by the host organism and operably linked to the polynucleotide encoding the polypeptide. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe gene to which they are operably linked, that is, with little or no control over gene expression. A large number of promoters, recognized by a variety of potential host cells, are well known.

Suitable promoters for use with yeast hosts, bacterial hosts, and insect hosts are well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

The vector may contain one or more elements that facilitate expression when the vector is integrated into the host cell genome. Examples include an EASE element (Aldrich et al. 2003 Biotechnol Prog. 19:1433-38) and a matrix attachment region (MAR). MARs mediate structural organization of the chromatin and may insulate the integrated vector from “position” effects. Thus, MARs are particularly useful when the vector is used to create stable transfectants. A number of natural and synthetic MAR-containing nucleic acids are known in the art, e.g., U.S. Pat. Nos. 6,239,328; 7,326,567; 6,177,612; 6,388,066; 6,245,974; 7,259,010; 6,037,525; 7,422,874; 7,129,062.

After the vector has been constructed and the polynucleotide has been inserted into the proper site of the vector, the completed vector may be inserted into a suitable host cell for amplification and/or polypeptide expression. The transformation of an expression vector into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. The host cells can be transfected transiently or the host cells can be transfected stably. These methods and other suitable methods are well known to the skilled artisan, and are set forth, for example, in Sambrook et al., 2001, supra.

For long-term, high-yield production of recombinant proteins, stable expression is often preferred. For example, cell lines that stably express the engineered heavy and light chains of the antigen-binding polypeptide construct can be prepared. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA or polynucleotide, engineered cells are allowed to grow for 1-2 days in an enriched medium, and then are switched to a selective medium. The selectable marker in the recombinant plasmid confers resistance to the selection and allows cells to stably integrate the plasmid into their chromosomes and grow to form foci that in turn can be cloned and expanded into cell lines.

A number of selection systems can be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk-, hgprt- or aprt-cells, respectively. Also, antimetabolite resistance can be used as the basis of selection for dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes.

A host cell, when cultured under appropriate conditions, produces the antigen-binding polypeptide construct that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. A host cell may be eukaryotic or prokaryotic. For example, expression in a bacterial system will produce an unglycosylated product and expression in yeast will produce a glycosylated product. Eukaryotic host cells that possess the cellular machinery for proper processing of the primary transcript (e.g., glycosylation, and phosphorylation) of the gene product can be used.

Mammalian cell lines available as hosts for expression are well known in the art and include, but are not limited to, immortalized cell lines available from the American Type Culture Collection (ATCC®) and any cell lines used in an expression system known in the art can be used to make the recombinant polypeptides described herein. In general, host cells are transformed with a recombinant expression vector that comprises DNA encoding the antigen-binding polypeptide construct. Among the host cells that may be employed are prokaryotes, yeast or higher eukaryotic cells. Prokaryotes include gram negative or gram positive organisms, for example E. coli or bacilli. Higher eukaryotic cells include insect cells and established cell lines of mammalian origin. Examples of suitable mammalian host cell lines include the COS-7 line of monkey kidney cells (ATCC® CRL 1651) (Gluzman et al., 1981, Cell 23:175), L cells, C127 cells, 3T3 cells (ATCC® CCL 163), Chinese hamster ovary (CHO) cells, or their derivatives such as Veggie CHO and related cell lines which grow in serum-free media (Rasmussen et al., 1998, Cytotechnology 28:31), HeLa cells, BHK (ATCC® CRL 10) cell lines, and the CV1/EBNA cell line derived from the African green monkey kidney cell line CV1 (ATCC® CCL 70) as described by McMahan et al., 1991, EMBO J. 10:2821, human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, it is possible to produce the polypeptide in lower eukaryotes such as yeast or in prokaryotes such as bacteria. Suitable yeasts include Saccharomyces cerevisiae, Schizosaccharomyces pombe, Kluyveromyces strains, Candida, or any yeast strain capable of expressing heterologous polypeptides. Suitable bacterial strains include Escherichia coli, Bacillus subtilis, Salmonella typhimurium, or any bacterial strain capable of expressing heterologous polypeptides.

If the antigen-binding polypeptide construct is produced in yeast or bacteria, it may be desirable to modify the product produced therein, for example by phosphorylation or glycosylation of the appropriate sites, in order to obtain a functional product. Such covalent attachments can be accomplished using known chemical or enzymatic methods. The antigen-binding polypeptide construct can also be produced by operably linking the set of polynucleotides to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac™ kit), and such methods are well known in the art, as described in Summers and Smith, Texas Agricultural Experiment Station Bulletin No. 1555 (1987), and Luckow and Summers, Bio/Technology 6:47 (1988). Appropriate cloning and expression vectors for use with bacterial, fungal, yeast, and mammalian cellular hosts are described by Pouwels et al. (Cloning Vectors: A Laboratory Manual, Elsevier, New York, 1985).

In certain embodiments, cell-free protein expression systems can be utilized to co-express polypeptides (e.g., heavy and light chain polypeptides) from the set of polynucleotides without the use of living cells. Instead, all components needed to transcribe DNA to RNA and translate the RNA to protein (e.g. ribosomes, tRNAs, enzymes, cofactors, amino acids) are provided in solution for use in vitro. In certain embodiments, the in vitro expression requires (1) the genetic template (mRNA or DNA) encoding the heavy and light chain polypeptides and (2) a reaction solution containing the necessary transcriptional and translational molecular machinery. In certain embodiments, cell extracts substantially supply components of the reaction solution, for instance: RNA polymerases for mRNA transcription, ribosomes for polypeptide translation, tRNA, amino acids, enzymatic cofactors, an energy source, and cellular components essential for proper protein folding. Cell-free protein expression systems can be prepared using lysates derived from bacterial cells, yeast cells, insect cells, plant cells, mammalian cells, human cells or combinations thereof. Such cell lysates can provide the correct composition and proportion of enzymes and building blocks required for translation. In some embodiments, cell membranes are removed to leave only the cytosolic and organelle components of the cell.

Several cell-free protein expression systems are known in the art as reviewed in Carlson et al. (2012) Biotechnol. Adv. 30:1185-1194. For example, cell-free protein expression systems are available based on prokaryotic or eukaryotic cells. Examples of prokaryotic cell-free expression systems include those from E. coli. Eukaryotic cell-free protein expression systems are available based on extracts from rabbit reticulocytes, wheat germ, and insect cells, for example. Such prokaryotic and eukaryotic cell-free protein expression systems are commercially available from companies such as Roche, Invitrogen, Qiagen, and Novagen. One skilled in the art would readily be able to select suitable cell-free protein expression systems that would produce polypeptides (e.g., heavy chain and light chain polypeptides) that are capable of pairing with each other. Further, the cell-free protein expression system can also be supplemented with chaperones (e.g. BiP) and isomerases (e.g. disulphide isomerase) to improve the efficiency of IgG folding.

### Co-Expression of Heavy Chains and Light Chains

The engineered immunoglobulin heavy chains and light chains of the antigen-binding polypeptide construct described herein can be co-expressed in mammalian cells, as noted above. In one embodiment, the immunoglobulin heavy chains and immunoglobulin light chains of the antigen-binding polypeptide construct are co-expressed in a host cell. Thus, in the case of a bispecific antigen-binding polypeptide construct, two immunoglobulin heavy chains and two immunoglobulin light chains are co-expressed in a host cell. However, alternate methods of producing bispecific antigen-binding polypeptide constructs that do not rely on the use of a single clonal or transient cell line expressing all four chains are also known in the art (Gramer, et al. (2013) mAbs 5, 962; Strop et al. (2012) J Mol Biol 420, 204). These methods rely on a post production arm exchange under redox conditions of the two pairs of light and heavy chain involved in the formation of bispecific antibody (Redox production). In this approach the HILL and H2L2 heterodimers can be expressed in two different cell lines to independently produce the two heterodimers. Subsequently, the two heterodimers are mixed under select redox conditions to achieve re-association of the two unique heavy chain H1 and H2 to form the bispecific antigen-binding polypeptide construct comprising H1L1H2L2.

Although preferential pairing is driven mainly by the incorporation of the Mab design set amino acid modifications into the immunoglobulin heavy and light chain polypeptides, the amount of correctly-paired heterodimers may further be optimized by varying the ratio of the polynucleotides encoding each polypeptide to each other, as shown in the Examples.

### Testing of Antigen-Binding Polypeptide Constructs

As described above, the antigen-binding polypeptide constructs comprise a first heterodimer comprising H1 and L1, as well as a second heterodimer comprising H2 and L2, where L1 is a lambda light chain, and L2 is a kappa light chain, and H1 and H2 are distinct from each other. One or more of H1, L1, H2, and L2 comprise amino acid modifications that promote preferentially pairing of H1 with L1 as compared to L2 and of H2 with L2 as compared to L1.

The first Fab region of the HILL heterodimer and the second Fab region of the H2L2 heterodimer are able to bind to antigen with an affinity that is similar to the corresponding wt first Fab region or wt second Fab region. The first Fab region of the HILL heterodimer and the second Fab region of the H2L2 heterodimer also exhibit thermal stability that is comparable to that of the corresponding wt first Fab region or wt second Fab region.

The affinity of each heterodimer of the heterodimer pair for its respective antigen can be tested as described below. The thermal stability of each heterodimer of the heterodimer pair can also be tested as described below.

In one embodiment, one heavy chain is co-expressed with two different light chains in a LCCA design set as described above, where the heavy chain preferentially pairs with one of the two light chains. In another embodiment, two unique heavy chains are co-expressed with two unique light chains, where each heavy chain preferentially pairs with one of the light chains.

### Methods to Measure Preferential Pairing

The degree of preferential pairing can be assessed, for example, by using the methods described below and in the examples. Preferential pairing can be assessed in the context of LCCA design sets (H1L1L2, or H2L1L2) or Mab design sets (H1L1H2L2).

In one embodiment, a Light Chain Competition Assay (LCCA) can be used to assess preferential pairing in the context of LCCA design sets. Co-owned patent application PCT/US2013/063306, filed Oct. 3, 2013, describes various embodiments of LCCA and is herein incorporated by reference in its entirety for all purposes. The method allows quantitative analysis of the pairing of heavy chains with specific light chains within the mixture of co-expressed proteins and can be used to determine if one particular immunoglobulin heavy chain selectively associates with either one of two immunoglobulin light chains when the heavy chain and light chains are co-expressed. The method is briefly described as follows: At least one heavy chain and two different light chains are co-expressed in a cell, in ratios such that the heavy chain is the limiting pairing reactant. The heavy chains and light chains may be tagged to facilitate detection. The secreted proteins may be separated from the cell, and the immunoglobulin light chain polypeptides bound to heavy chain are isolated from other secreted proteins to produce an isolated heavy chain paired fraction. The amount of each different light chain in the isolated heavy chain fraction is then detected, and the relative amount of each different light chain in the isolated heavy chain fraction is analyzed to determine the ability of the heavy chain to selectively pair with one of the light chains. Further details regarding an embodiment of this method are provided in the Examples.

In another embodiment, preferential pairing is assessed in the context of Mab design sets, where H1, L1, H2 and L2 are co-expressed. In this embodiment, one or more of H1, L2, H2, and L2 may be tagged to facilitate detection and analysis. The resulting species of paired heavy chains and light chains are assessed using LCMS (Liquid chromatography-Mass spectrometry), based on differences in the molecular weight of each species. An antigen activity assay could also be used to quantify relative heterodimer populations containing each light chain whereby the degree of binding measured (relative to controls) would be used to estimate each respective heterodimer population.

### Thermal Stability

The thermal stability of the heterodimers can be determined according to methods known in the art. The melting temperature of each heterodimer is indicative of its thermal stability. The melting point of the heterodimer can be measured using techniques such as differential scanning calorimetry (Chen et al (2003) Pharm Res 20:1952-60; Ghirlando et al (1999) Immunol Lett 68:47-52). Alternatively, the thermal stability of the heterodimer can be measured using circular dichroism (Murray et al. (2002) J. Chromatogr Sci 40:343-9).

### Affinity for Antigen

The binding affinity of the heterodimers for their respective antigens and the off-rate of the interaction can be determined by competitive binding assays according to methods well known in the art. One example of a competitive binding assay is a radioimmunoassay comprising the incubation of labeled antigen (e.g., 3H or 125I with a molecule of interest (e.g., heterodimers of described here) in the presence of increasing amounts of unlabeled antigen, and the detection of the molecule bound to the labeled ligand. The affinity of the heterodimers for the antigen and the binding off-rates can be determined from the saturation data by Scatchard analysis.

The kinetic parameters of a heterodimer described herein can also be determined using surface plasmon resonance (SPR) based assays known in the art (e.g., BIAcore™ kinetic analysis). For a review of SPR-based technology see Mullet et al., 2000, Methods 22:77-91; Dong et al., 2002, Review in Mol. Biotech., 82:303-23; Fivash et al., 1998, Current Opinion in Biotechnology 9:97-101; Rich et al., 2000, Current Opinion in Biotechnology 11:54-61. Additionally, any of the SPR instruments and SPR based methods for measuring protein-protein interactions described in U.S. Pat. Nos. 6,373,577; 6,289,286; 5,322,798; 5,341,215; 6,268,125 are contemplated in the methods of the invention. FACS can also be used to measured affinity, as is known in the art.

### Pharmaceutical Compositions

Also provided herein are pharmaceutical compositions comprising the antigen-binding polypeptide constructs described herein. Such compositions comprise a therapeutically effective amount of the antigen-binding polypeptide construct, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In certain embodiments, the composition comprising the antigen-binding polypeptide construct is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

The amount of the composition described herein which will be effective in the treatment, inhibition and prevention of a disease or disorder associated with aberrant expression and/or activity of a therapeutic protein can be determined by standard clinical techniques. In addition, in vitro assays can optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

### Uses of Antigen-Binding Polypeptide Constructs

As described above, the antigen-binding polypeptide constructs described herein are obtained from parent antibodies where each heterodimer of the antigen-binding polypeptide construct corresponds to one of the parent antibodies, and has been engineered to incorporate amino acid modifications that promote preferential pairing of the immunoglobulin heavy and light chains that make up the heterodimers. Accordingly, the antigen-binding polypeptide constructs described herein can be used in the treatment or prevention of the same disease, disorder, or infection that the parent antibody or combination of parent antibodies is used for.

In another embodiment, the antigen-binding polypeptide constructs described herein can also be utilized in combination with other therapeutic agents known in the art for the treatment or prevention of a cancer, autoimmune disease, inflammatory disorders or infectious diseases. In a specific embodiment, the antigen-binding polypeptide constructs described herein can be used in combination with monoclonal or chimeric antibodies, lymphokines, or hematopoietic growth factors (such as, e.g., IL-2, IL-3 and IL-7), which, for example, serve to increase the number or activity of effector cells which interact with the molecules and, increase immune response. The antigen-binding polypeptide constructs described herein can also be utilized in combination with one or more drugs used to treat a disease, disorder, or infection such as, for example anti-cancer agents, anti-inflammatory agents or anti-viral agents.

### Generation of Bispecific Antibodies Using a Mab Design Set Library

In one embodiment, the Mab design sets described herein can be employed to produce bispecific antigen-binding polypeptide constructs. The Mab design sets described herein can be utilized in the form of a Mab design set library, where the Mab design set library comprises Mab design sets that show utility in promoting preferential pairing to form bispecific antigen-binding polypeptide constructs. In one embodiment, Mab design set libraries are represented by Mab design sets included in Table 4A and Table 4B. In one embodiment, the Mab design set library is represented by the Mab design sets in one or more of Tables 10-A1 to 10-A12, and 10-B1 to 10-B10. In another embodiment, the Mab design set library is represented by one or more of Tables 10-A1 to 10-A12. In one embodiment, the Mab design set library is represented by one or more of Tables 10-B1, 10-B2, 10-B3, 10-B4, 10-B6, 10-B8, and 10-B10. The Mab design set library can be used to produce an antigen-binding polypeptide construct starting from two parent antibodies (i.e., Mab1 and Mab2) as follows. For the sake of illustration, Mab1 comprises a lambda Fab and comprises the immunoglobulin heavy chain polypeptide H1 and the immunoglobulin light chain polypeptide L1, while Mab2 comprises a kappa Fab and comprises the immunoglobulin heavy chain polypeptide H2 and the immunoglobulin light chain polypeptide L2.

The Mab design set amino acid modifications (H1L1H2L2) of the Mab design set library are introduced into the immunoglobulin heavy and light chain of Mab1 (H1 and L2) and the immunoglobulin heavy and light chain of Mab2 (H2 and L2). H1, L1, H2, and L2 are then co-expressed and the amount of the correctly paired bispecific antigen-binding polypeptide construct is determined. One or more Mab design sets of the Mab design set library may be individually tested or screened to determine which provides the desired amount of bispecific antigen-binding polypeptide construct. Each heterodimer of the bispecific antigen-binding polypeptide construct can be further tested to assess the ability of each heterodimer to bind to antigen or to assess its thermal stability, as described herein. Additional properties that can be assessed include solubility, aggregation, kon and koff rates, ability to withstand exposure to acids, bases, oxidation, freeze/thaw cycles, agitation, pressure etc. of the bispecific antigen-binding polypeptide construct compared to the parent antibodies or Fab regions of the parent antibodies. The latter properties can be impacted by the complementarity determining regions (CDRs) of an antibody of interest, and thus may be tested for each bispecific antigen-binding polypeptide construct generated.

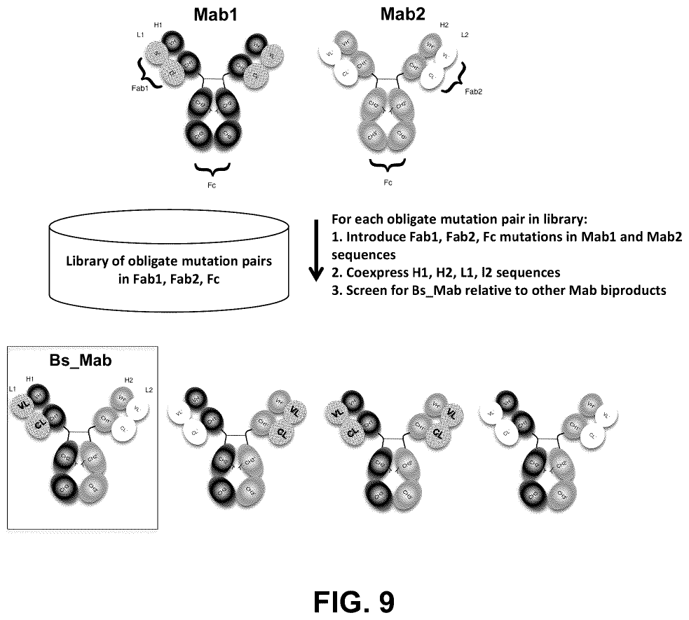

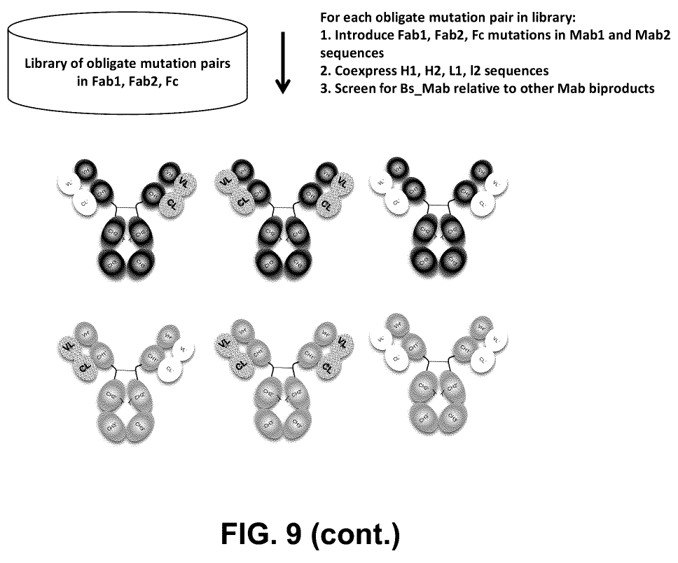

In some embodiments the amount of correctly paired bispecific antigen-binding polypeptide construct is assessed by LCMS. In some embodiments the amount of correctly paired bispecific antigen-binding polypeptide construct is assessed by charge based separation techniques such as a capillary isoelectric focusing (cIEF) technique or a chromatographic technique. The procedure for preparation of a bispecific antigen-binding polypeptide construct from Mab1 and Mab2 using a library of Mab design sets is shown schematically in FIG. 9.

In one embodiment, the Mab design set library is stored on a computer-readable storage medium to facilitate use of the Mab design set library to design bispecific antigen-binding polypeptide constructs.

### Computer Implementation

In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device can be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Below are examples of specific embodiments for making and using the antigen-binding polypeptide constructs described herein. The examples are offered for illustrative purposes only, and are not intended to limit the scope of the disclosure in any way. Efforts have been made to ensure accuracy with respect to numbers used (e.g., amounts, temperatures, etc.), but some experimental error and deviation should, of course, be allowed for.

The constructs and methods described herein can be prepared and carried out employing, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992).

### EXAMPLES

### Example 1: Molecular Modeling and Computer Guided Engineering of the Fab Interface

A structure- and computational molecular modeling-guided approach was used to produce a kappa-lambda (K-L) design library for the preparation of bispecific antibodies where one Fab has a kappa light chain and the other Fab has a lambda light chain (i.e. a kappa-lambda system, or K-L system). The K-L design library comprises designs with amino acid modifications in the heavy and light chains of the Fabs that promote preferential formation of the desired bispecific antibody when these heavy and light chains are co-expressed. The K-L design library takes advantage of inherent differences between kappa and lambda light chains in bispecific antibodies and is hence optimized for kappa-lambda systems. The K-L design library was generated by studying the structure of representative Fabs, with D3H44 (anti-tissue factor antibody) as a representative Fab containing a kappa light chain (kappa Fab), and CAT-2200 (anti-IL-17A antibody) as a representative Fab containing a lambda light chain (lambda Fab), but the library can be used in the context of other bispecific K-L system antibodies or fragments thereof to identify designs that exhibit the desired pairing specificity in the antibodies of interest.

The representative Fabs were chosen based on the criteria indicated in Table 1. These criteria included that the Fabs be human or humanized, have commonly used VH and VL subgroups and contain minimal framework region mutations. Also, pairwise 3D superposition with available non-redundant (90% sequence identity threshold) kappa and lambda structures was performed (structures obtained from the RSCB PDB, a database maintained by Rutgers University (Camden, NJ) and the University of California at San Diego (San Diego, CA), see the internet at www.rcsb.org. Together with the other parameters described in Table 1, low H-L cross domain RMSDs (root-mean-square deviations) for VH-VL or CH1-CL was used to select a representative structure for each one of the kappa and lambda systems. After selection of D3H44 (PDB ID 1JPT) and CAT-2200 (PDB ID 2VXS) as representative Fabs, an in silico structural analysis of these Fab interfaces was carried out to identify and understand residues important for interactions between heavy and light chains, using a two-pronged approach.

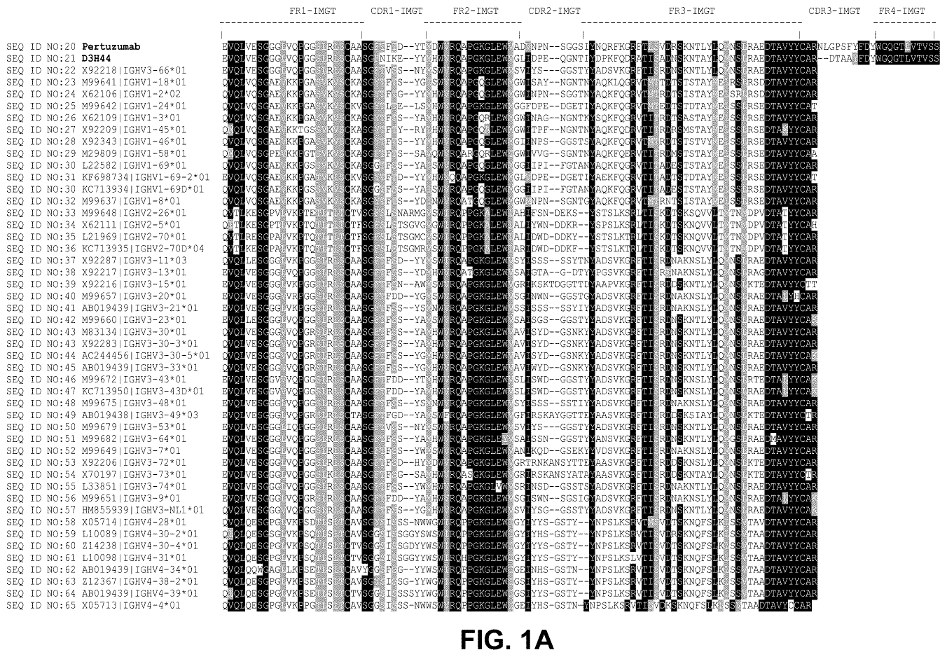

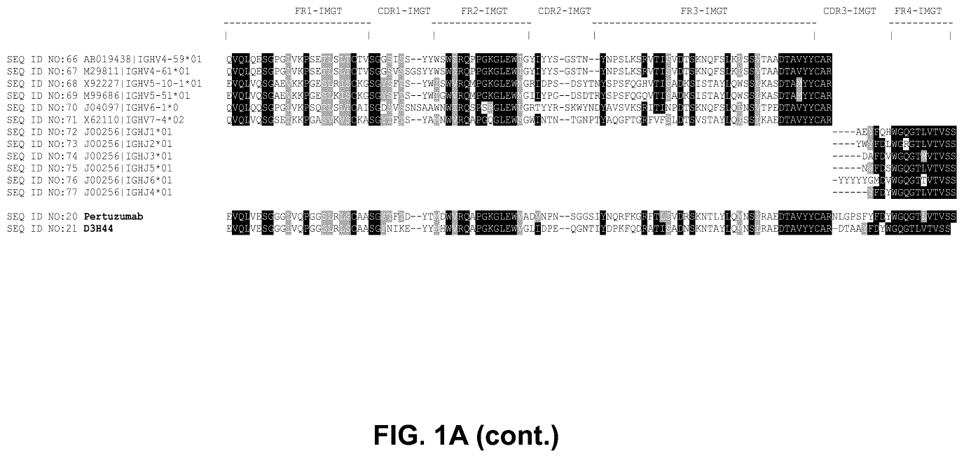

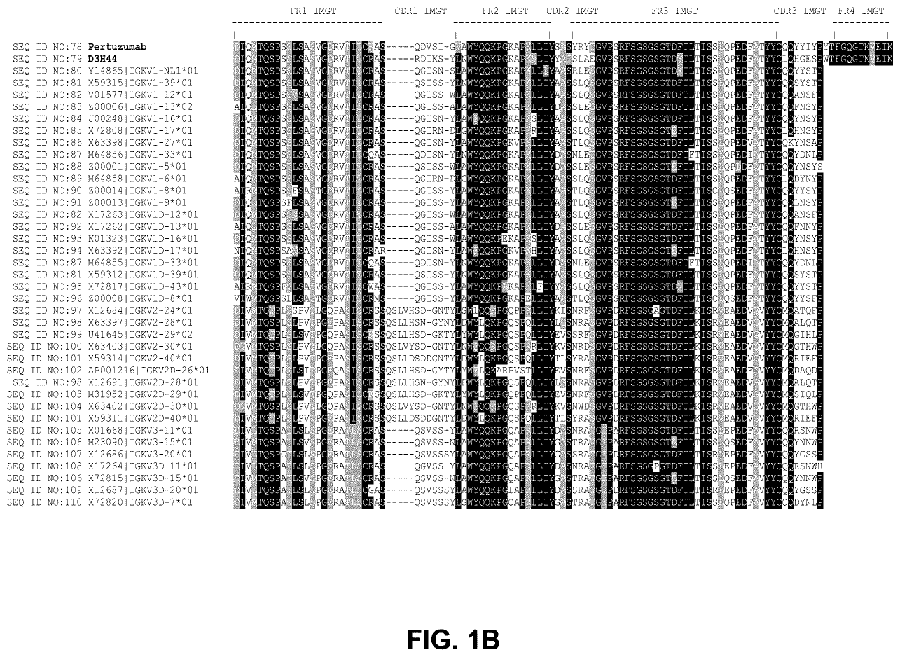

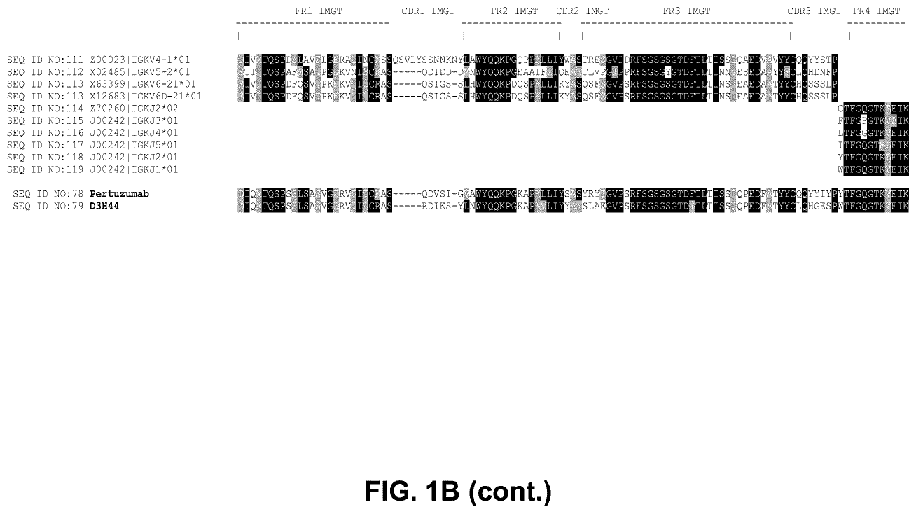

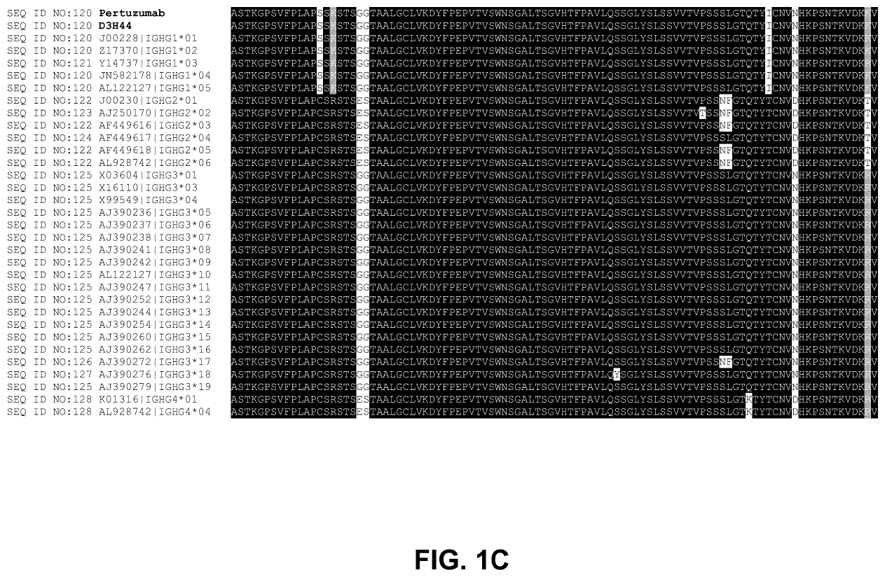

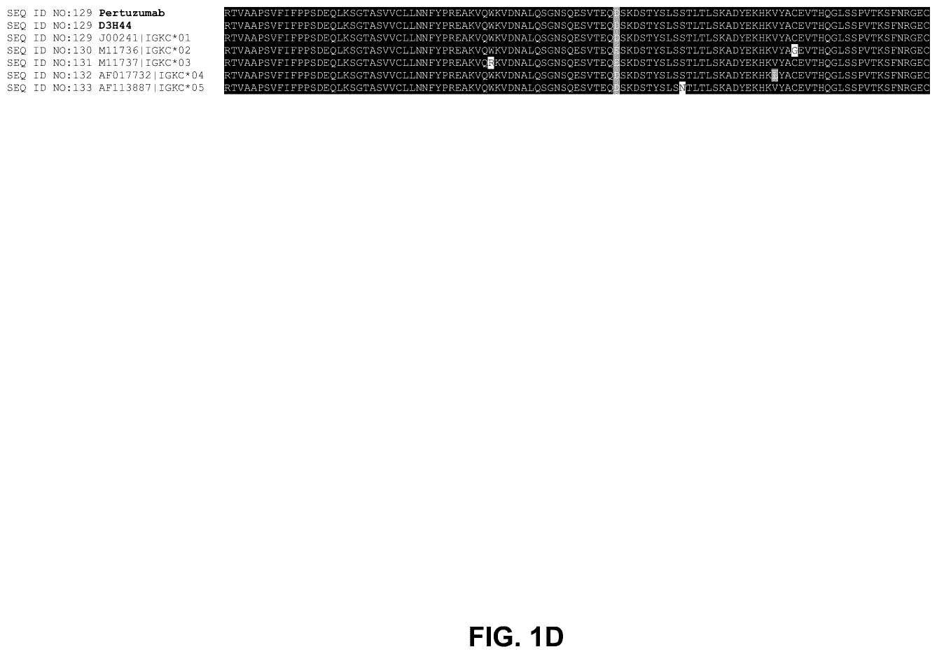

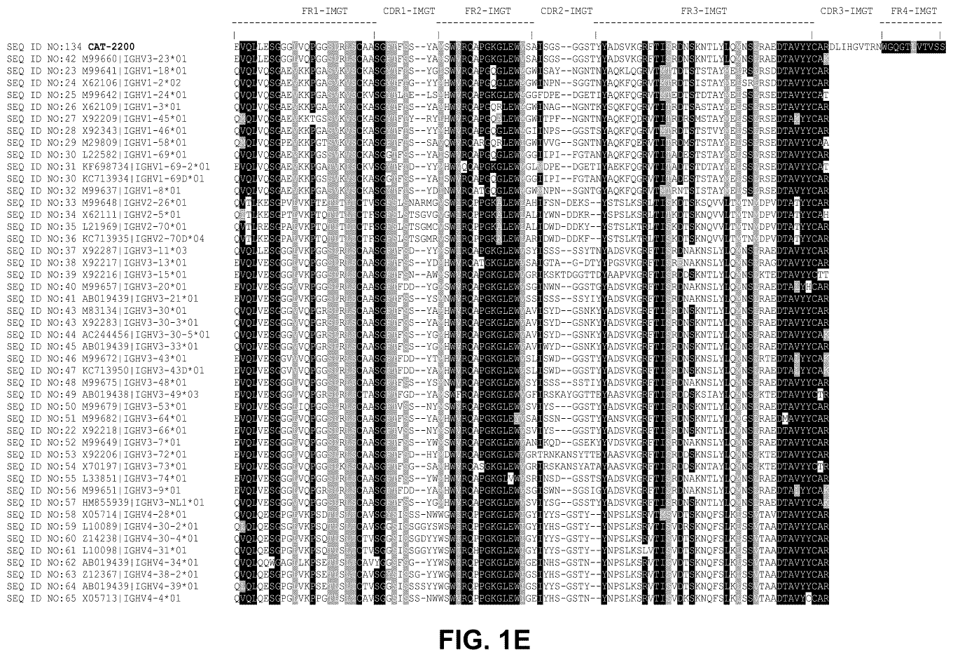

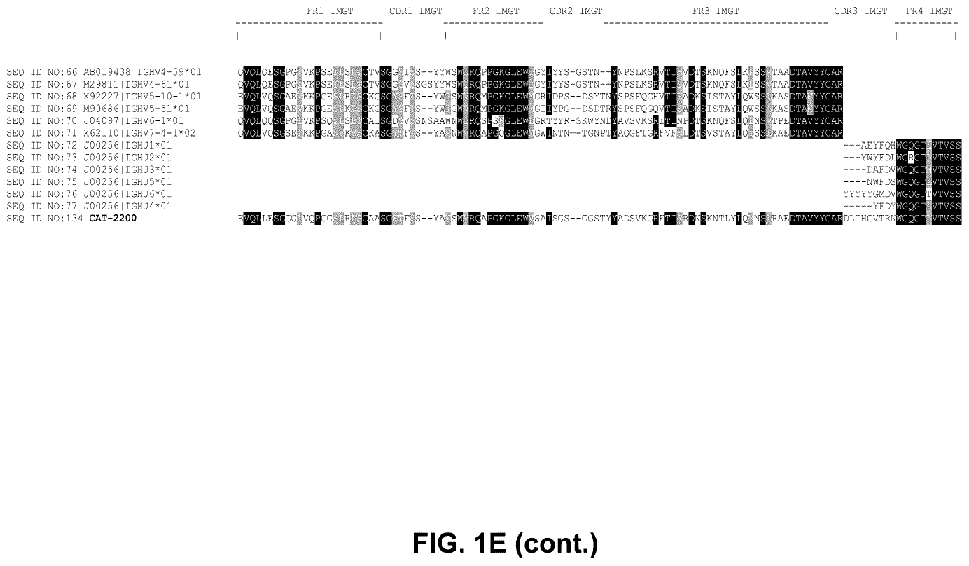

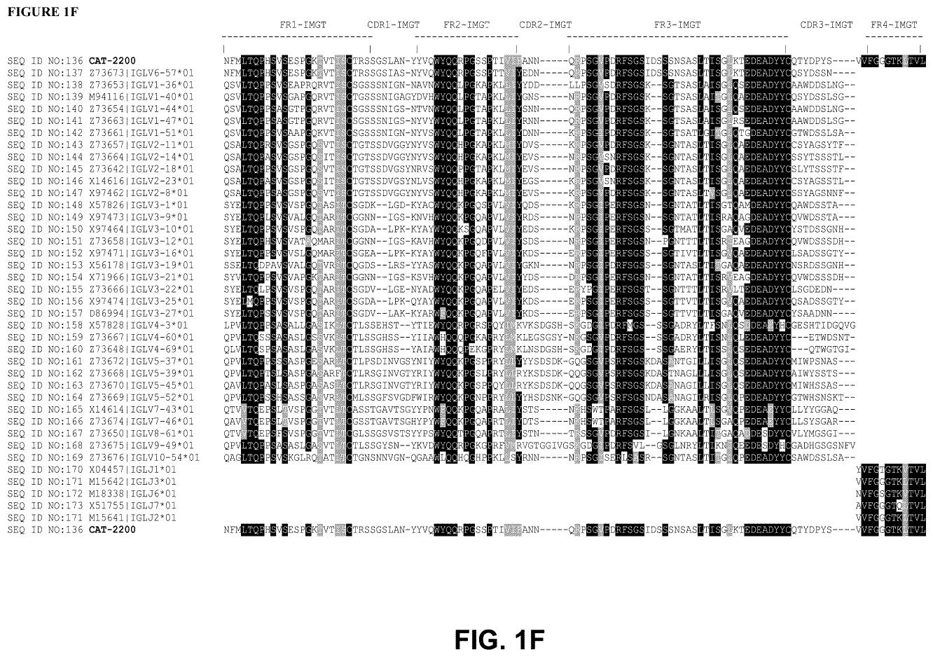

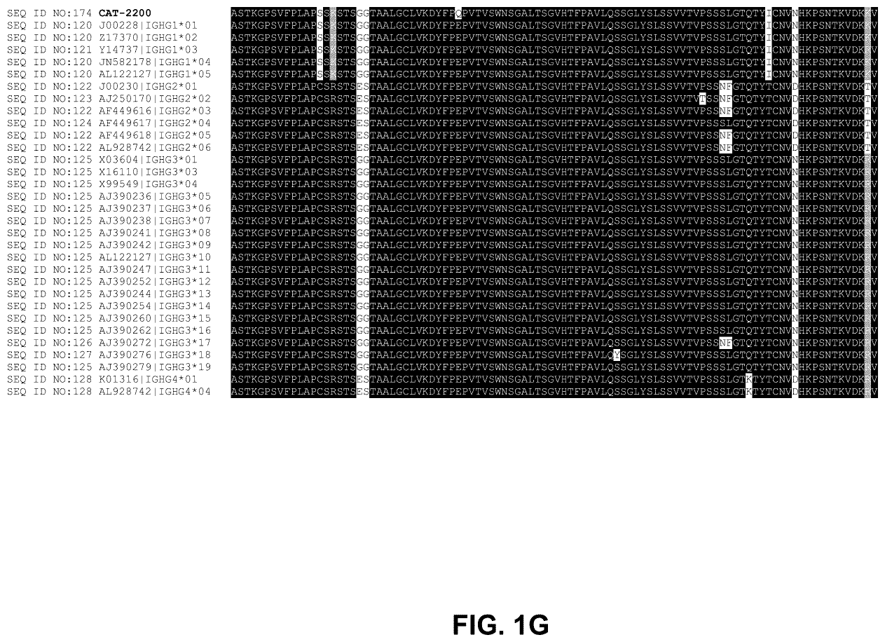

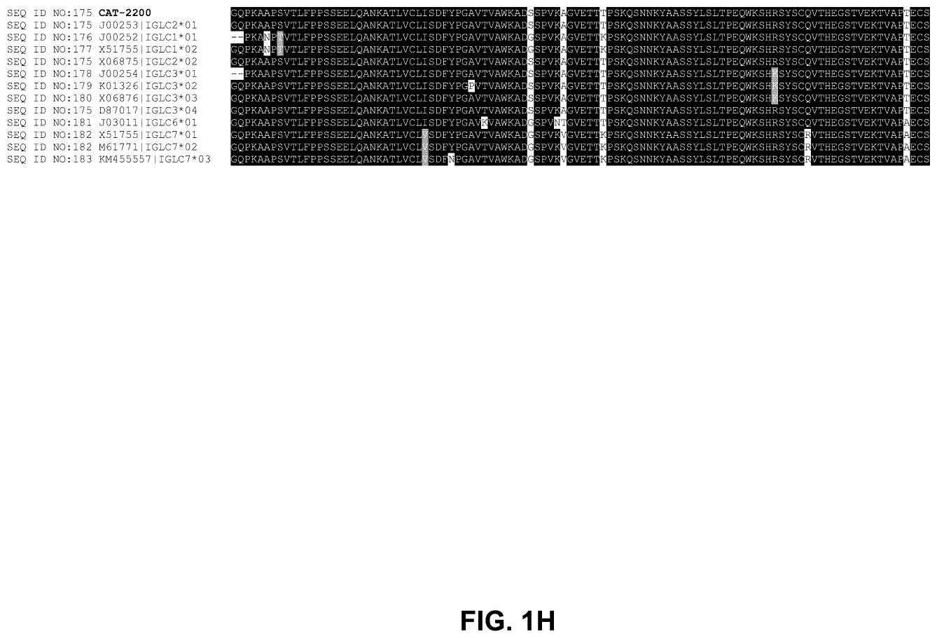

First, global analysis of the sequence conservation across the Fab variable and constant interfaces was carried out via sequence and structure alignments of known antibodies. An alignment of constant and variable domain sequences from various antibody subgroups, compared to the sequences of D3H44 and the anti-HER2 antibody pertuzumab (both containing kappa light chains) and CAT-2200 (containing a lambda light chain) is shown in FIG. 1A-1H. FIG. 1A and FIG. 1E depict an alignment comparing representative human VH germline subgroups with that of D3H44/Pertuzumab and CAT-2200, respectively. FIG. 1B depicts an alignment comparing D3H44/Pertuzumab with representative human kappa VL germline subgroups. FIG. 1C and FIG. 1G depict an alignment comparing human CH1 allele sequences with that of D3H44/Pertuzumab and CAT-2200, respectively. FIG. 1D depicts an alignment comparing D3H44/Pertuzumab with human kappa allele sequences. FIG. 1F depicts an alignment comparing CAT-2200 with representative human lambda VL germline subgroups. FIG. 1H depicts an alignment comparing CAT-2200 with human lambda allele sequences. This analysis revealed that pertuzumab and D3H44 show high degree of sequence conservation with kappa constant and variable domain germlines, while CAT-2200 shows high degree of sequence conservation with lambda constant and variable domain germlines. These alignments also exemplify the higher degree of conservation in the constant domains of both heavy and light chains, making designs in the constant domain interface more likely to be transferable to other antibodies.

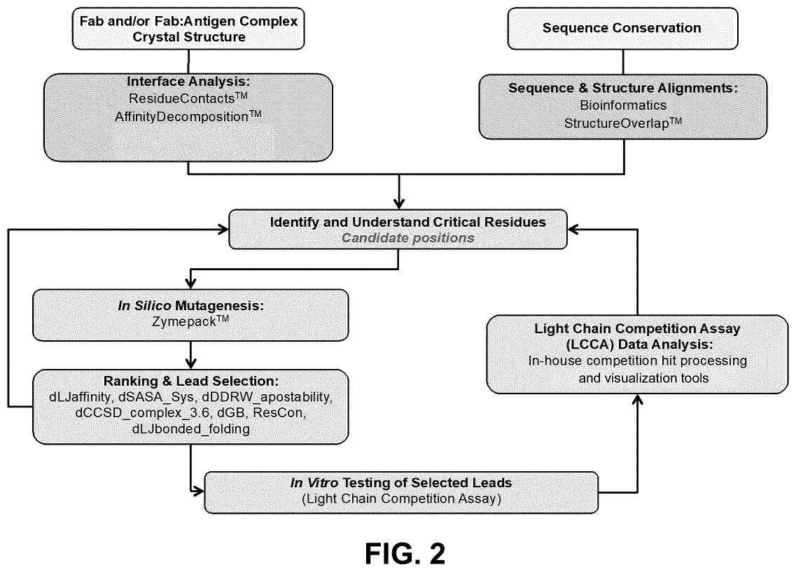

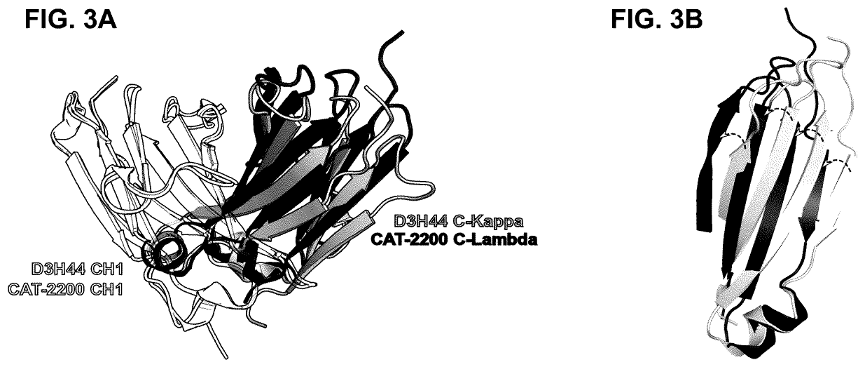

The second approach involved the analysis of the D3H44 and CAT-2200 crystal structure interfaces using a number of molecular modeling tools as shown in FIG. 2 (e.g. RESIDUECONTACTS™ and AFFINITYDECOMPOSITION™). To improve transferability to other antibodies or fragments the analysis focused on the constant domain where sequence conservation is higher (see FIG. 1A-1H). These analyses were used to identify differences in hotspot positions (key interface residues) between the representative kappa Fab (D3H44) and lambda Fab (CAT-2200) structures, as shown in Table 2. There are noticeable conformational differences in the constant domain between CH1-CL (kappa) and CH1-CL (lambda) structures. Superposing available Fab structures on the CH1 domain showed that CL (kappa) structures adopt a very similar conformation and form a tight cluster. CL (lambda) conformations, however, tend to reside into two distinct clusters: one closer to the kappa orientation (kappa-like cluster) and one more discrepant orientation (lambda cluster). By this analysis D3H44 represents a typical kappa structure and CAT-2200 represents a typical lambda structure (lambda cluster). FIGS. 3A and 3B exemplifies the typical constant domain kappa-lambda conformational difference using D3H44 and CAT-2200 as representative structures. These differences seem to stem mainly from a rigid body movement of the light constant domain relative to the CH1 domain, which changes the nature of the H-L interface in the constant domain of kappa when compared with a lambda system. The identified hotspot discrepancies (Table 2) as well as the above-mentioned conformational differences (FIGS. 3A and 3B) served as the starting point for the engineering of H-L pairing designs for a bispecific system containing a kappa Fab and a lambda Fab. The numbering of amino acids in the parent D3H44 and CAT-2200 sequences according to Kabat is provided in Table 3A and Table 3B.

Next, potential mutations at the hotspot positions as well as positions neighboring the hotspots of interest in the 3D crystal structure were simulated and identified via in silico mutagenesis and packing/modeling with ZYMEPACK™. ZYMEPACK™ is a software suite that, given an input structure and a set of mutations, will alter the residue types in the input structure according to the supplied mutations, and generate a new structure that is an approximation to the physical structure of the mutant protein. Additionally, ZYMEPACK™ evaluates the properties of the mutant protein by computing a variety of quantitative metrics. These metrics include measures of steric and electrostatic complementarity, which may correlate with the stability, binding affinity, or heterodimeric specificity of the mutant protein.

By exploiting interface discrepancies, mutations were introduced to promote selective pairing of the desired or preferred polypeptide chains or heterodimers while disfavoring the formation of incorrectly paired or mispaired polypeptide chains or heterodimers. For example, in order to prepare a bispecific antibody with one kappa Fab and one lambda Fab, four polypeptide chains are required. The kappa Fab comprises heavy chain 1 (H1) and kappa light chain 1 (L1), while the lambda Fab comprises heavy chain 2 (H2) and lambda light chain 2 (L2). In this case, the desired H-L pairs would be H1L1 and H2L2, while the incorrect pairs would be H1L2 and H2L1. Note that the designation/numbering of the polypeptide chains here is arbitrary. Thus, mutations that favor the paired CH1-CL (kappa) interface (H1L1) over the mispaired CH1-CL (lambda) interface (H1L2) and mutations that favor the paired CH1-CL (lambda) interface (H2L2) over the mispaired CH1-C L (kappa) interface (H2L1) were identified to generate kappa Fab-lambda Fab tailored designs in the constant domain. Using computational methods including ZYMEPACK™, steric complementarity was modeled and also computed on the basis of energy factors such as van der Waals packing, cavitation effects and close contact of hydrophobic groups. Similarly, electrostatic interaction energies were modeled and evaluated on the basis of coulomb interactions between charges, hydrogen bonds, and desolvation effects. Both the preferred heavy and light chain pair models H1L1 and H2L2, and the incorrect pair models H1L2 and H2L1 obtained by introducing the mutations of interest were simulated to compute the relative steric and electrostatic scores. This allowed the determination of whether a particular mutation set led to favorable energies i.e. greater steric and/or electrostatic complementarity for the preferred heavy-light chain pairs relative to the incorrect pairs. The computed steric and electrostatic energies are components of the free energy associated with the light and heavy chain pairing. Hence greater steric and electrostatic complementarity is indicative of a larger free energy change associated with the pairing of the desired pair relative to the pairing of the incorrect pair. The greater steric and/or electrostatic complementarity results in preferential (selective) pairing of the desired heavy and light chains relative to the steric penalty and/or electrostatic repulsion for the incorrect pair.

### Example 2: Selection and Description of Designs

The approach described in Example 1 was used to design heavy chain-light chain heterodimer pairs (i.e. H1L1 and H2L2) that exhibit selective or preferential pairing when one of the H-L heterodimer pairs contains a kappa light chain and the other contains a lambda light chain. The heterodimers were designed in pairs, referred to as a “Mab design” or “Mab design set,” and include a set of amino acid substitutions on H1, L1, H2, and L2 chains that promote preferential pairing. The Mab design sets were initially tested as “LCCA designs” where one heavy chain of the Mab design set was co-expressed with the two light chains of the Mab design set, one kappa and one lambda, in order to assess relative pairing. The amino acid substitutions described throughout the Examples are identified with reference to Table 3A and Table 3B (for pertuzumab and CAT-2200 Fabs), using the Kabat numbering system as described in Kabat and Wu, 1991; Kabat et al, Sequences of proteins of immunological interest. 5th Edition-US Department of Health and Human Services, NIH publication no. 91-3242, p 647 (1991), unless otherwise indicated. For reference, however, Tables 22A, 22B, and 22C provide an identification of selected amino acid positions in the heavy chain, kappa light chain, and lambda light chain using IMGT, 1JPT, and EU numbering systems as applicable.

The Mab designs were packed onto a molecular model of D3H44 and CAT-2200 and metrics were calculated as described in Example 1. The top designs were then selected based on risk (minimizing effects on stability as well as immunogenicity) and impact (which takes into account the proposed strength of the drive pairing specificity). The top designs were then tested by a light chain competition assay (LCCA) to experimentally determine their pairing specificity (see Example 4). Although the Mab designs were identified using D3H44 and CAT-2200 as representative Fabs, they were tested in a K-L system using Pertuzumab as the kappa Fab and CAT-2200 as the lambda Fab (pertuzumab-CAT-2200 K-L system). As shown in FIG. 1C and FIG. 1D, D3H44 and Pertuzumab have identical sequences in the constant domain, allowing for seamless interconversion between the two systems. These Mab designs are referred to as K-L designs.

A second set of designs was also tested in the pertuzumab/CAT-2200 K-L system. These designs were proposed using representative designs that reflected the diversity of the designs from the kappa-kappa (K-K) design library described in Table 30 of International Patent Application Number PCT/CA2013/050914 (International Patent Publication No. WO2014/082179) as a starting point. These representative K-K-derived designs included a subset of designs that were either ported without modification to the K-L system where possible, or adapted to the kappa-lambda-system by modifying amino acid residues as necessary to account for sequence and structural differences between kappa and lambda light chains. Both types of representative K-K-derived designs are referred to as K-K-derived K-L designs.

Pairing specificity for K-L designs and K-K-derived K-L designs was experimentally assessed as LCCA designs by LCCA in the kappa-lambda system, as described in Example 4.

### Example 3: Preparation of Fab Constructs Encoding Pertuzumab IgG Heavy Chains, Pertuzumab IgG Light Chains, CAT-2200 IgG Heavy Chains, and CAT-2200 IgG Light Chains

The wild-type Fab heavy and light chains of the anti-HER2 antibody Pertuzumab and for the anti-IL17 antibody CAT-2200 were prepared as follows. The protein sequences of the pertuzumab Fab light chain (GenBank® Accession No. HC359025.1, SEQ ID NO:2) and heavy chain (GenBank® Accession No. HC359024.1, SEQ ID NO:1) were reverse translated to DNA, codon optimized for mammalian expression, and gene synthesized (SEQ ID NOs: 8 and 7, respectively). The protein sequences of the CAT-2200 Fab light chain (2VXS chain L, SEQ ID NO: 4) and heavy chain (2VXS chain H, SEQ ID NO:3) were taken from the PDB entry 2VXS, reverse translated to DNA, codon optimized for mammalian expression, and gene synthesized (SEQ ID NOs: 10 and 9, respectively). The polypeptide and DNA sequences of these antibody heavy and light chains are shown in Table 3C.

Light chain vector inserts, consisting of a 5′-EcoRI cutsite-HLA-A signal peptide-HA or FLAG tag-Light chain Ig clone-‘TGA or TAA stop’-BamH1 cutsite-3′, were ligated into a pTT5 vector (Durocher Y et al., Nucl. Acids Res. 2002; 30, No. 2 e9) to produce light chain expression vectors. The resulting light chain expression vectors were sequenced to confirm correct reading frame and sequence of the coding DNA. Likewise, heavy chain vector inserts, consisting of a 5′-EcoR1 restriction site-HLA-A signal peptide-heavy chain clone (terminating at T238; see Table 3A)-ABD2-His6tag-TGA stop-BamH1 cutsite-3′, were ligated into a pTT5 vector (ABD2=two copies of the albumin binding domain protein, in tandem) to produce heavy chain expression vectors. The resulting heavy chain expression vectors were also sequenced to confirm correct reading frame and sequence of the coding DNA. The various Pertuzumab or CAT-2200 Fab constructs containing amino acid substitutions for the Mab design sets were generated either by gene synthesis or by site-directed mutagenesis (Braman J, Papworth C & Greener A., Methods Mol. Biol. (1996) 57:31-44).

Heavy and light chains were tagged at the C- and N-termini respectively, in order to facilitate the assessment of preferential pairing via a competition assay-SPR screen (LCCA). The ABD2-His6 heavy chain tag allowed H-L complexes to be captured on an anti-His tag SPR chip surface, whilst FLAG and HA light chain tags allowed the relative L1 and L2 populations to be quantified.

### Example 4: Assessment of Preferential Pairing of Designed Fab Heterodimers by Light Chain Competition Assay (LCCA)

Constructs encoding CAT-2200 and Pertuzumab IgG heavy and light chains in the Fab format comprising amino acid modifications were prepared as described in Example 3. The ability of the constructs to preferentially pair to form the desired H-L heterodimer in the context of an LCCA design set (H1, L1, L2 or H2, L2, L1) was determined using a Light Chain Competition Assay (LCCA).

The LCCA quantifies the relative pairing of one heavy chain for at least two unique light chains, one kappa and one lambda, and can be summarized as follows. To determine pairing specificity for the lambda Fab, one CAT-2200 heavy chain Fab construct was co-expressed with one CAT-2200 light chain Fab construct (for the paired Fab product H1L1) and one Pertuzumab light chain Fab construct (for the mispaired Fab product H1L2) and the relative light chain pairing specificity (H1L1: H1L2) was determined from a competition assay-SPR screen, conducted in duplicate. Conversely, to determine pairing specificity for the kappa Fab, one Pertuzumab heavy chain Fab construct was co-expressed with one Pertuzumab light chain Fab construct (for the paired Fab product H2L2) and one CAT-2200 light chain Fab construct (for the mispaired Fab product H2L1) and the relative light chain pairing specificity (H2L2:H2L1) was determined from a competition assay-SPR screen, conducted in duplicate.

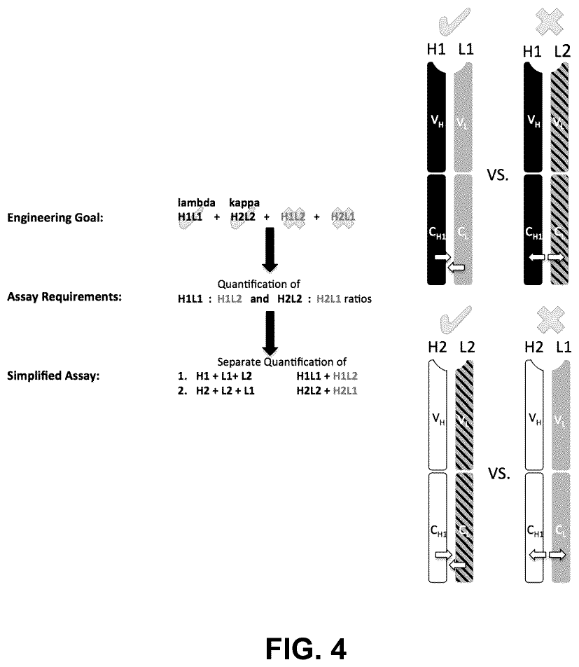

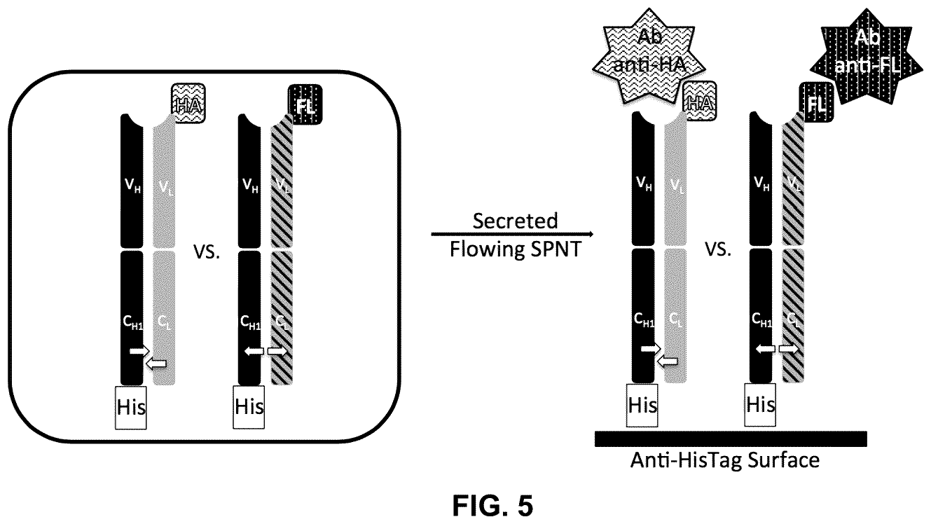

The LCCA assay was performed by concomitant expression of heavy and light chains at ratios of 1:1:1 (by weight) for H1:L1:L2 or H2:L2:L1, with the heavy chain being in limiting amounts. The amount of each heterodimer formed (i.e. H1L1 and H1L2 or H2L2 and H2L1) was determined by binding heavy chains to the SPR chip via a his-tag pull-down, followed by detection of the amount of each light chain tag (HA or FLAG) using antibodies specific for these tags. Subsequently, selected heterodimer hits were verified via a light chain competition assay verification whereby the L1: L2 DNA ratios were again checked for a 1:1 ratio, while keeping the heavy chain in limiting quantities. For these particular kappa-lambda system combinations the light chain tags can have an effect on the wild type LCCA pairing, leading to deviations from the expected neutral 50%: 50% ratio. For that reason the arrangement with the least amount of deviation from neutral was chosen by fusing the Pertuzumab light chain with the HA tag and the CAT-2200 light chain with the FLAG tag. A schematic representing the design of the assay is shown in FIG. 4. FIG. 5 depicts how the heavy chains and light chains are tagged and how preferential pairing is assessed. The experimental details of the LCCA are provided below.

### Transfection Method

LCCA designs comprising one heavy chain construct and two light chain constructs, prepared as described in Example 3, were transfected into CHO-3E7 cells as follows. CHO-3E7 cells, at a density of 1.7-2×10⁶ cells/ml, were cultured at 37° C. in FREESTYLE™ F17 medium (Invitrogen cat #A-1383501) supplemented with 4 mM glutamine and 0.1% KoliphorP188 (Sigma #K4894). A total volume of 2 ml was transfected with a total of 2 μg DNA using PEI-pro (Polyplus transfection #115-375) at a DNA: PEI ratio of 1:2.5. All transfections were performed using 333 ng of each heavy chain and each light chain of (i.e. H1:L1:L2 or H2:L2:L1=1:1:1 ratio), 300 ng of AKTdd pTT22 (a vector containing a constitutively active protein kinase B mutant), and 700 ng of ssDNA (salmon sperm DNA). Twenty-four hours after the addition of the DNA-PEI mixture, 0.5 mM Valproic acid (final concentration) and 1% w/v Tryptone (final concentration) were added to the cells which were then transferred to 32° C. Supernatants were tested for expression on day 7 by non-reducing SDS-PAGE analysis followed by Coommassie blue staining to visualize the bands.

### Competition Assay SPR Method

The degree of preferential heavy chain pairing to each of the two light chains in the LCCA designs was assessed using an SPR-based readout of unique epitope tags located at the N-terminus of each light chain. CAT-2200 LC construct contain a FLAG tag and Pertuzumab LC construct contain an HA tag.

Surface Plasmon resonance (SPR) supplies. GLC sensorchips, the Biorad ProteOn amine coupling kit (1-ethyl-3-(3-dimethylaminopropyl) carbodiimide hydrochloride (EDC), N-hydroxysulfosuccinimide (sNHS) and ethanolamine), and 10 mM sodium acetate buffers were purchased from Bio-Rad Laboratories (Canada) Ltd. (Mississauga, ON). 4-(2-hydroxyethyl)-1-piperazineethanesulfonic acid (HEPES) buffer, ethylenediaminetetraacetic acid (EDTA), and NaCl were purchased from from Sigma-Aldrich (Oakville, ON). 10% Tween® 20 solution was purchased from Teknova (Hollister, CA).

SPR biosensor assays. All surface plasmon resonance assays were carried out using a BioRad ProteOn XPR36 instrument (Bio-Rad Laboratories (Canada) Ltd. (Mississauga, ON)) with PBST running buffer (PBS Teknova Inc with 0.05% Tween20) at a temperature of 25° C. The anti-penta His™ capture surface was generated using a GLC sensorchip activated by a 1:5 dilution of the standard BioRad sNHS/EDC solutions injected for 140 s at 100 μL/min in the analyte (horizontal) direction. Immediately after the activation, a 25 μg/mL solution of anti-penta His™ antibody (Qiagen Inc.) in 10 mM NaOAc pH 4.5 was injected in the analyte (vertical) direction at a flow rate of 25 μL/min until approximately 3000 resonance units (RUs) are immobilized. Remaining active groups were quenched by a 140 s injection of 1M ethanolamine at 100 μL/min in the analyte direction, and this also ensures mock-activated interspots were created for blank referencing.

The screening of the heterodimers for binding to the anti-FLAG (Sigma Inc.) and anti-HA (Roche Inc.) monoclonal antibodies occurred in two steps: an indirect capture of the heterodimers onto the anti-penta His™ surface in the ligand direction followed by an anti-FLAG and anti-HA injection in the analyte direction. First, one injection of PBST for 30 s at 100 μL/min in the ligand direction was used to stabilize the baseline. For each heterodimer capture, unpurified heterodimers in cell-culture media were diluted to 4% in PBST. One to five heterodimers or controls (i.e. controls containing either 100% HA-light chain or 100% FLAG-light chain) were simultaneously injected in individual ligand channels for 240 s at flow 25 μL/min, resulting in a saturating heterodimer capture of approximately 300 to 400 RUs onto the anti-penta His surface. The first ligand channel was left empty to use as a blank control if required. This heterodimer capture step was immediately followed by two buffer injections in the analyte direction to stabilize the baseline, and then 5 nM anti-FLAG and 5 nM anti-HA were each injected in duplicate at 50 μL/min for 120 s with a 180 s dissociation phase, resulting in a set of binding sensorgrams with a buffer reference for each of the captured heterodimer. The heterodimers were regenerated by an 18 s pulse of 0.85% phosphoric acid for 18 s at 100 L/min to prepare the anti-penta His™ surface for the next injection cycle. Sensorgrams were aligned and double-referenced using the buffer blank injection and interspots, and the resulting sensorgrams were analyzed using ProteOn Manager software v3.0.

### Data Analysis and Description of LCCA Metrics

The assessment of preferential pairing for each design, present in the H1L1H2L2 format, was assessed by two complementary LCCA design sets, one for the H1L1L2 combination and the other for the H2L1L2 combination. Each LCCA design set is denoted with a ‘unique identifier’ (for example, 10629 or 10695). Each Mab design set (H1L1H2L2) is consequently identified by the LCCA design set numbers for the two constituent LCCAs (e.g. 10629-10695).

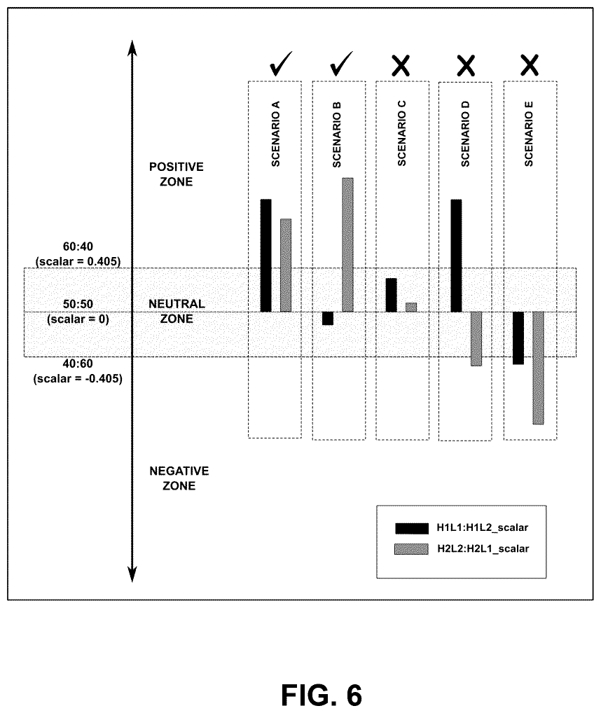

The LCCA design sets (H1L1L2 or H2L1L2) of each Mab design set were tested in the LCCA, and design sets of interest were identified based on the following inclusion criteria. In order to be considered a design of interest, the design must contain at least one positive LCCA result above the neutral zone (>60:40 correctly paired: mispaired ratio) while the other complementary LCCA had to be above the lower limit of the neutral zone (the 40:60 correctly paired: mispaired ratio) as illustrated by FIG. 6. The neutral zone was defined as the region between 40%: 60% and 60%: 40% correctly paired: mispaired ratios using the median normalized values for H1-L1: H1-L2 and H2-L2:H2-L1. For example, Scenarios A and B in FIG. 6 represent designs that pass the inclusion criteria. Scenario B is included because H2L2:H2L1 LCCA is above the neutral zone and H1L1: H1L2 LCCA inside the neutral zone (not below it). Scenario C represents a design where both LCCA results are positive, but neither are above the neutral zone, and hence is not included. Scenarios D and E represent designs that are filtered out because at least one of the LCCA results is below the neutral zone, even if the other LCCA is above the neutral zone (see Scenario D). Tables 4A (K-L designs) and 4B (K-K-derived K-L designs) list the Mab design sets that met these criteria, while Tables 5A and 5B show the results of the LCCA for these designs. In Tables 4A and 4B and all subsequent tables listing amino acid substitutions of the LCCA design sets or Mab design sets, the absence of a substitution in the table, or a “-” indicates that the polypeptide chain does not include an amino acid substitution that promotes preferential pairing.

The performance of each LCCA design set is described by two scalar values, ln(r1/f1) or ln(r2/f2), where r1 and r2 correspond to the median values of the ratios H1L1: H1L2 and H2L2:H2L1, respectively, and f1 and f2 correspond to the respective median values of ratios H1L1: H1L2 and H2L2:H2L1. The normalized scalar values are generated by subtracting the contribution of the WT (wild-type) system from the design; ln(r1/f1)_{design}-ln(r1/f1)_{WT} or ln(r2/f2)_{design}-ln(r2/f2)_{WT}. For the wild type CAT-2200 (HC) competed against the CAT-2200 (LC-FLAG) plus the Pertuzumab (LC-HA) the ln(r1/f1_{WT}=−0.48. For the wild type Pertuzumab (HC) competed against the Pertuzumab (LC-HA) plus the CAT-2200 (LC-FLAG) the ln(r2/f2)_{WT}=0.66. This procedure effectively removed any existing WT pairing bias and normalizes the specificity in comparison to a neutral 50%: 50% L1: L2 WT system. These values are reported in columns 2 and 5 in Tables 5A and 5B.

In addition, the normalized scalar values were also converted back into a pure normalized median ratio H1L1: H1L2 and H2L2:H2L1. This conversion effectively normalized the LCCA ratios to 100% as it was observed for some designed Fabs that the total amount of H1L1 and H1L2, or H2L2 and H2L1 significantly differed from 100%. This discrepancy in total light chain percentage is believed to be due in part to the occurrence of variable non-specific binding during initial heterodimer capture on the SPR chip. These values are reported in columns 4 and 7 in Tables 5A and 5B.

Furthermore, the scalar range for each normalized LCCA of a design (H1L1: H1L2 and H2L2:H2L1 experiments) as well as the range for the normalized ratio of correctly paired to mispaired Fab heterodimers (the range is the same of percentage of L1 and percentage of L2 in the normalized ratio) is shown. For results reported from a single measurement the range values are marked NA (not applicable). These values are reported in columns 3 and 6 in Tables 5A and 5B.

For reference, Table 23 presents the correspondence between % of paired: mispaired heterodimers and the corresponding LCCA scalar value.

### Results

The LCCA results are shown in Tables 5A and 5B in the context of Mab design sets. Table 5A provides the LCCA data relating to the K-L designs described in Example 2, while Table 5B provides the LCCA data relating to the K-K-derived K-L designs also described in Example 2. Note that all LCCA experiments were performed on constructs containing the inter-chain Fab disulfide bond located in the constant domain (H/C233-L/C214, Kabat numbering).

Table 4A lists 231 K-L designs that met the inclusion criteria. The LCCA data relating to these designs is shown in Table 5A. Table 4B lists 44 K-K-derived K-L designs that met the inclusion criteria and Table 5B shows the LCCA data relating to these designs. Designs that maintain 100% identity between the original K-K design library from Table 30 in the above-noted PCT Application No. PCT/CA2013/050914 and the new kappa-lambda system (i.e. were ported without modification) are identified with an asterisk (*) in Table 4B. Most of the designs in Table 4B have amino acid modifications in the constant region only, with a few of the designs also incorporating amino acid modifications in the variable region. These designs were proposed to further drive pairing specificity while also favoring transferability to other antibody systems.

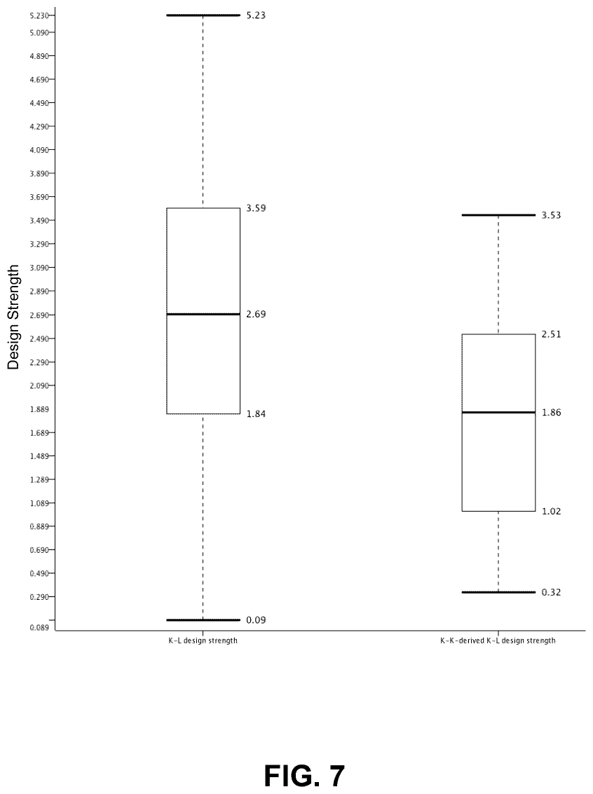

Design strength can be defined as the sum of the two LCCA scalar values for a particular design. By comparing the design strength of K-K-derived K-L designs with the K-L designs it was apparent that those designs tailored specifically for the K-L system tended to show better pairing specificity than the K-K designs transferred into a K-L system, as summarized in FIG. 7. The median design strength for the K-K-derived K-L designs was 1.86, with a maximum design strength of 3.53. The median design strength for the K-L designs was 2.69, with a maximum design strength of 5.23.

### Example 5: Scale Up of Designed Fab Heterodimers for Biophysical Characterization

Correctly paired heterodimers H1L1 or H2L2, as indicated in the unique identifier sets (Tables 4A and 4B), were scaled up (typically to 20 ml) and purified as follows in order to test for thermal stability and antigen binding. For these experiments, the heavy chains of the Fabs were expressed without the ABD2 tag, while the light chains were expressed with the same HA or FLAG tag used in the LCCA. The heavy and light chains of each heterodimer were expressed in 20 ml cultures of CHO-3E7 cells. CHO-3E7 cells, at a density of 1.7-2×10⁶ cells/ml, were cultured at 37° C. in FREESTYLE™ F17 medium (Invitrogen cat #A-1383501) supplemented with 4 mM glutamine and 0.1% Koliphor P188 (Sigma #K4894). A total volume of 20 ml were transfected with a total of 20 μg DNA using PEI-pro (Polyplus cat #115-375) at a DNA: PEI ratio of 1:2.5. Twenty-four hours after the addition of the DNA-PEI mixture, 0.5 mM Valproic acid (final concentration) and 1% w/v Tryptone (final concentration) were added to the cells which were then transferred to 32° C.

Cells were centrifuged 7 days after transfection, and heterodimers were purified from supernatant by affinity capture using IgG-CH1 CaptureSelect™ (Life Technologies™, Catalog No.: 194-320-005) at 4° C. as follows. Supernatants were diluted to 20-25% cell culture supernatant in equilibration buffer (Dulbecco's phosphate buffered saline (DPBS) without Calcium, Magnesium, and phenol red (HyClone™ #SH30028.02)) and then incubated with mixing for 16 hours with IgG-CH1 CaptureSelect™, also previously equilibrated with the equilibration buffer. The resin was then collected by centrifugation, transferred to a 96 well-fritted plate, washed with equilibration buffer three times. Bound samples were eluted with 1 to 4 bed volumes of elution buffer: 100 mM glycine pH 2.6. Eluted samples were collected by centrifugation into a 96-well UV-transparent receiver plate, each well containing neutralizing buffer: 1M Tris™ pH 9.0 at 10% of elution volume.

Following purification, heterodimer expression was assessed by non-reducing High Throughput Protein Express assay using Caliper LabChip GXII (Perkin Elmer #760499). Procedures were carried out according to HT Protein Express LabChip User Guide version 2 LabChip GXII User Manual, with the following modifications. Heterodimer samples, at either 2 μl or 5 μl (concentration range 5-2000 ng/μl), were added to separate wells in 96 well plates (BioRad #HSP9601) along with 7 μl of HT Protein Express Sample Buffer (Perkin Elmer #760328). The heterodimer samples were then denatured at 70° C. for 15 mins. The LabChip instrument was operated using the HT Protein Express Chip (Perkin Elmer #760499) and the Ab-200 assay setting. After use, the chip was cleaned with MilliQ water and stored at 4° C.

Inclusion of Mab design set modifications did not have a significant impact on protein expression. Expression levels obtained for CAT-2200 Fabs containing design modifications are 0.45 mg/20 mL expression (SD of 0.16 mg), compared with 0.64 mg/20 mL expression (SD of 0.1 mg) for the wild type Fab. Pertuzumab Fabs bearing the design modifications express at 0.39 mg/20 mL expression (SD of 0.14 mg), compared with 0.4 mg/20 mL expression (SD of 0.03 mg) for the wild type Fab. Caliper analysis showed purity levels comparable to the WT Fab.

### Example 6: Antigen Affinity Measurements of Designed Fab Heterodimers

The ability of CAT-2200 Fab heterodimers to bind to IL-17A and Pertuzumab Fab to bind to HER2-ECD was assessed in order to determine whether the amino acid substitutions had any effect on the ability of the heterodimer to bind to antigen. Fab heterodimers were prepared as described in Example 5. The affinity of each Fab heterodimer for its antigen was determined by SPR as follows.

SPR supplies. GLC sensorchips, the Biorad ProteOn amine coupling kit (EDC, sNHS and ethanolamine), and 10 mM sodium acetate buffers were purchased from Bio-Rad Laboratories (Canada) Ltd. (Mississauga, ON). PBS running buffer with 0.05% Tween20 (PBST) was purchased from Teknoca Inc. (Hollister, CA). Recombinant HER2 protein was purchased from eBioscience (San Diego, CA). Goat polyclonal anti-human Fc antibody was purchased from Jackson Immuno Research Laboratories Inc. (West Grove, PA). Recombinant human IL-17A was purchased from R&D Systems (Minneapolis, MN).

CAT-2200: IL-17A affinity determination. All surface plasmon resonance assays were carried out using a BioRad ProteOn XPR36 instrument (Bio-Rad Laboratories (Canada) Ltd. (Mississauga, ON)) with PBST running buffer at a temperature of 25° C. The IL-17A surface was generated using a GLC sensorchip activated by a 1:10 dilution of the standard BioRad sNHS/EDC solutions injected for 140 s at 100 μL/min in the ligand (vertical) direction. Immediately after the activation, a 2 microg/mL solution of IL-17A in 10 mM NaOAc pH 4.5 was injected in the ligand (vertical) direction at a flow rate of 25 microL/min until approximately 50 resonance units (RUs) were immobilized. Remaining active groups were quenched by a 140 s injection of 1M ethanolamine at 100 μL/min also in the ligand direction.

The screening of the designed Fabs for binding to IL-17A was performed by injection of purified CAT-2200 Fabs in the analyte (horizontal) direction. Firstly, two buffer injections for 30s at 100 microL/min in the analyte (horizontal) direction were used to stabilize the baseline. Five concentrations of a three-fold dilution series of each CAT-2200 designed Fab (60 nM, 20 nM, 6.7 nM, 2.2 nM, 0.74 nM) with a blank buffer control were simultaneously injected at 50 microL/min for 120 s with a 15 minute dissociation phase, resulting in a set of binding sensorgrams with a buffer reference. CAT-2200: IL-17A complexes on the SPR surface are dissociated and the IL-17A surface regenerated to prepare for the next injection cycle by two pulses of 0.85% phosphoric acid for 18 s at 100 microL/min. Sensorgrams were aligned and double-referenced using the buffer blank injection and interspots, and the resulting sensorgrams were analyzed using ProteOn Manager software v3.1. The double-referenced sensorgrams were fit to the Langmuir binding model.

Pertuzumab: HER2 affinity determination. All surface plasmon resonance assays were carried out using a BioRad ProteOn XPR36 instrument (Bio-Rad Laboratories (Canada) Ltd. (Mississauga, ON)) with PBST running buffer at a temperature of 25° C. The anti-penta-His™ antibody (Qiagen) capture surface was generated using a GLC sensorchip activated by a 1:10 dilution of the standard BioRad sNHS/EDC solutions injected for 140 s at 100 L/min in the analyte (horizontal) direction. Immediately after activation, a 10 microg/mL solution of anti-penta-His antibody in 10 mM NaOAc pH 4.5 was injected in the ligand (vertical) direction at a flow rate of 25 μL/min until approximately 3000 resonance units (RUs) were immobilized. Remaining active groups were quenched by a 140 s injection of 1M ethanolamine at 100 μL/min in the analyte direction to ensure mock-activated interspots are created for blank referencing.

The screening of the designed Fabs for binding to HER2 occurred in two steps: an indirect capture of the designed Fabs onto the anti-penta-His™ antibody surface in the ligand direction followed by the simultaneous injection of 5 concentrations of purified HER2 antigen and one buffer blank for double referencing in the analyte direction. Firstly, one buffer injection for 30 s at 100 microL/min in the ligand direction was used to stabilize the baseline. For each designed Fab capture, designed Fabs were diluted to 2 microg/mL in PBST. One to five designed Fabs or controls were simultaneously injected in individual ligand channels for 240 s at flow 25 μL/min. This resulted in a capture of approximately 400 to 600 RUs onto the anti-human Fc surface. The first ligand channel was left empty to use as a blank control if required. This capture step was immediately followed by two buffer injections in the analyte direction to stabilize the baseline, and then 100 nM, 33.3 nM, 11.1 nM, 3.7 nM and 0.41 nM Her2 along with a buffer blank was simultaneously injected at 50 μL/min for 120 s with a 1200s dissociation phase. The captured antibody surfaces were regenerated by an 18 s pulse of 0.85% phosphoric acid at 100 μl/min to prepare for the next injection cycle. Sensorgrams were aligned and double-referenced using the buffer blank injection and interspots, and the resulting sensorgrams were analyzed using ProteOn Manager software v3.1. The double-referenced sensorgrams were fit to the 1:1 binding model.

Results. Antigen affinity (KD) values for designed Fab heterodimer samples are reported in Table 6A (K-L designs) and Table 6B (K-K-derived K-L designs) in the context of design pairs. The KD values of H1L1 and H2L2 Fabs are included in columns 2 and 4 (KD of H1L1 CAT-2200 Fab heterodimer and H2L2 Pertuzumab Fab heterodimer, respectively), and 3 and 5 (change in KD of H1L1 or H2L2 Fab heterodimer compared to its respective wild-type). KD values were determined only for Fab heterodimer samples that exhibited a Fab heterodimer capture of at least 100 RU. The reference KD value for wild-type CAT-2200 bound to IL-17A used for comparison to the designed heterodimers was 0.273 nM. This value is a median value based on multiple measurements of the wild-type CAT-2200 Fab heterodimer where the light chain contains a FLAG tag. Similarly, the reference KD value for wild-type pertuzumab bound to HER2 was 8.055 nM. This value is a median value based on multiple measurements of the wild-type pertuzumab Fab heterodimer where the light chain contains an HA tag. In Table 6, the difference in KD with respect to wild type antigen binding affinity is shown using the calculation−(log(KD_design)−log(KD_wt)), such that positive values indicate lower KD values whereas negative values indicate increased KD values of the Fab heterodimer compared with wild type binding affinity for antigen. NB indicates that no binding was detected. Note that some Fab heterodimers lack measured KD values and are hence marked as not determined (ND). In some of these cases, the Fab heterodimers were assessed but technical difficulties (such as low Fab heterodimer capture in the SPR experiment, i.e. less than 100 RU), prevented accurate determinations of KD values.

For the majority of the designed Fab heterodimers tested, amino acid substitutions had little or no impact on KD values. Three designs (Unique identifiers 10881-11412, 10883-11236, and 10888-11415) on the K-L design library showed a >5-fold decrease in KD on the CAT-2200 Fab. On the K-K-derived K-L design library, one of the Pertuzumab Fab heterodimers (designs containing the 10724 code in the Unique identifier) showed a >5-fold decrease in KD on the Pertuzumab side.

### Example 7: Thermal Stability Measurements of Designed Fab Heterodimers by DSF

Differential Scanning Fluorescence (DSF) was used to measure the thermal stability of correctly paired heterodimers in comparison to that of wild type, unmodified heavy chain-light chain pair. Although DSF is known to be a less precise method for measuring melting temperatures, it was used here because it is a high throughput method of measuring thermal stability and thus allowed for some measurement of thermal stability to be obtained for a large number of Fab heterodimers. Fab heterodimers were prepared as described in Example 5.

### Measurement of Thermal Stability by DSF

The thermal stability of designed Fab heterodimer pairs was measured using DSF as follows. Each heterodimer was purified as described in Example 5 and diluted to 0.5 mg/mL in DPBS (Dulbecco's phosphate buffered saline, HyClone Cat #SH30028.02). For the majority of samples, a working stock of Sypro Orange gel stain (Life Technologies Cat #S-6650) was prepared by diluting 4 μL of Sypro Orange gel stain to 2 ml DPBS. The DSF samples were prepared by adding 14 μL of 0.5 mg/mL protein to 60 μL of the diluted Sypro Orange gel stain working stock. However, for proteins that had less than 0.5 mg/mL, each DSF sample were prepared by adding 14 μL of the undiluted protein to 60 μL of a working stock of Sypro Orange dye (that was diluted to 1:1500 in DPBS). DSF analysis was then conducted, in duplicate, on 20 μl aliquots using the Rotor-Gene 6000 qPCR instrument (QiaGen Inc). Each sample was scanned from 30° C. to 94° C. using 1° C. intervals with a 10 second equilibrium between each step and a 30 second wait time at the start. An excitation filter of 470 nM and emission filter of 610 nM with a gain of 9 was used. Data was analyzed with the Rotor-Gene 6000 software using the maxima value from the first derivative of the denaturation curve as the Tm. The remaining DSF samples were prepared and analyzed similarly, with the following protocol modifications that do not alter the measured Tm values: 1) the working stock was prepared by diluting 1 μL of Sypro Orange gel stain to 2 ml DPBS, 2) 30 μl aliquots were analyzed and 3) a gain of 10 was used.

DSF results for designed Fab heterodimer samples are reported in Tables 6A (K-L designs) and 6B (K-K-derived K-L designs), in the context of Mab design sets. Thermal stability of H1L1 and H2L2 Fabs are included in columns 6 and 8 (H1L1 refers to CAT-2200 Fab heterodimer and H2L2 to Pertuzumab Fab heterodimer) of Tables 6A and 6B. For Fab heterodimers where repeats were conducted, the reported Tm value is the median value. Comparisons of the designed Fab heterodimer Tm values with respect to the Tm value of the wild-type Fab heterodimer are reported in column 7 for the CAT-2200 H1L1 and in column 9 for the Pertuzumab H2L2. The median Tm for wild-type CAT-2200 Fab construct containing a FLAG tag was determined to be 76.9° C. The median Tm for wild-type Pertuzumab Fab heterodimer containing a HA tag, was determined to be 82.4° C. Given throughput limitations in the scale up procedure for the large number of Fab heterodimers generated by the Mab design sets, DSF measurements were performed by sampling a subset of designed Fab heterodimers. For that reason, some Fab heterodimers lack Tm values measured by DSF and are hence marked as not determined (ND). The majority of the designs tested showed good thermostability, within the range of expected Tm values commonly observed for wild-type Fabs. Designed Fab heterodimers containing the mutations K145T_V177D_S188D in the HC and S176K_Y178L in the lambda LC or S176K_T178L in the kappa LC, negatively affected the Tm values by >10° C. in the Fab heterodimer containing those mutations.

### Example 8: Thermal Stability Measurements of Designed Fab Heterodimers by DSC

To complement the measurement of thermal stability by DSF, the more precise method of differential scanning calorimetry (DSC) was also used to measure the thermal stability of correctly paired, designed Fab heterodimers in comparison to that of wild type, unmodified heavy chain-light chain pair. Fab heterodimers were prepared as described in Example 5.

### Measurement of Thermal Stability by DSC

The thermal stability of designed Fab heterodimer pairs was measured using DSC as follows. 400 μL of purified samples primarily at concentrations of either 0.2 mg/ml or 0.4 mg/mL in PBS were used for DSC analysis with a VP-Capillary DSC (GE Healthcare). At the start of each DSC run, 5 buffer blank injections were performed to stabilize the baseline, and a buffer injection was placed before each sample injection for referencing. Each sample was scanned from 20 to 100° C. at a 60° C./hr rate, with low feedback, 8 sec filter, 5 min preTstat, and 70 psi nitrogen pressure. The resulting thermograms were referenced and analyzed using Origin 7 software.

Thermal stability of H1L1 and H2L2 Fabs are included in columns 10 and 12 (H1L1 refers to CAT-2200 Fab heterodimer and H2L2 to Pertuzumab Fab heterodimer, respectively) of Table 6. For each Fab heterodimer where repeats were conducted, the reported Tm value is the median value. Comparisons of the designed Fab heterodimer Tm values with respect to the Tm value of the wild-type Fab heterodimer are reported in column 11 for the CAT-2200 H1L1 (wild type CAT-2200 Fab construct containing a FLAG tag, with a median Tm of 70.7° C.) and column 13 for the Pertuzumab H2L2 (wild type Pertuzumab Fab construct containing a HA tag, with a median Tm of 78.7° C.). Given the high sample amounts requirement and low throughput nature of DSC, measurements were performed by sampling a subset of designs. For this reason, some Fab heterodimers lack Tm values measured by DSC and are hence marked as not determined (ND). All of the designed Fab heterodimers assayed by DSC showed good thermostability. The DSC-derived Tm values were generally lower than those observed by DSF (Example 7).

### Example 9: Optimization of Designs

The performance of the K-L designs and K-K-derived K-L designs in the pertuzumab/CAT-2200 system, as shown in Tables 5A and 5B, respectively, was examined in order to guide further understanding of the ability of the designs to drive selective pairing. This examination enabled a subsequent cycle of design optimization to improve pairing specificity where possible and/or increase the stability of the heterodimers. Design optimization included modification of the K-L designs and K-K-derived K-L designs at a particular substituted amino acid residue to assess the effect of alternate amino acid substitution at that residue, adding amino acid substitutions at additional amino acid residues, and/or removing amino acid substitution at a particular residue (i.e. converting it back to the wild-type residue). The design process and selection of designs was performed as described in Examples 1 and 2, and the resultant designs were tested in the pertuzumab/CAT-2200 K-L system.

An additional set of K-L designs was proposed using an alternate method of increasing the diversity of designs with improved pairing specificity, in which selected constant domain designs from Table 4A and 4B, as well as selected constant domain optimization design candidates described in the previous paragraph were combined with two K-K variable domain designs. The two K-K variable domain designs are included in Table 4B, and have the unique identifiers 10621-10733 and 10623-10745. These designs did not promote strong pairing specificity on their own and did not affect antigen binding, but have been shown to improve pairing specificity when combined with certain Fab constant domain designs in a kappa-kappa antibody system. This additional set of K-L designs also included combinations of constant domain designs only, where the constant domain designs are selected from Table 4A, 4B and optimization design candidates described in the previous paragraph.

Finally, a further additional set of K-K-derived K-L designs were proposed using representative designs from the K-K design library described in Table 5 of PCT Application No. PCT/IB2015/054107 as a starting point, and tested in the pertuzumab/CAT-2200 K-L system. As described in Example 2, the representative K-K-derived designs were adapted to the kappa-lambda-system by modifying amino acid residues as necessary to account for sequence and structural differences between kappa and lambda light chains.

The pairing specificity of the additional K-L designs and K-K-derived K-L designs described in this Example was experimentally assessed by LCCA in the pertuzumab-CAT-2200 K-L system as described in Example 10.

### Example 10: Preparation and Assessment of Preferential Pairing of Designed Fab Heterodimers by LCCA

The pertuzumab and CAT-2200 Fab constructs comprising amino acid substitutions corresponding to the Mab design sets were prepared as described in Example 3. As described in Example 4, all LCCA experiments were performed on constructs containing the inter-chain Fab disulfide bond located in the constant domain (H/C233-L/C214, Kabat numbering). In order to assess preferential pairing exhibited by the Mab design sets, the preferential pairing exhibited by the corresponding LCCA design sets was measured as described in Example 4, with the following exceptions.

Assessment of preferential pairing in the LCCA design sets was carried out by LCCA as described in Example 4 except that transfection was carried out with heavy and light chains at ratio of 1:1:3 (by weight) for H1:L1:L2 or H2:L2:L1, instead of 1:1:1, with the heavy chain present in limiting amounts, and the mispaired light chain present in excess. Since the designs described in Example 9 were optimized versions of those described in Tables 4A and 4B, or constituted combinations of two designs, some of which already exhibit relatively high pairing specificity, it was possible that further improvement in such specificity (if attained) might not be detectable in the LCCA at the H1:L1:L2 or H2:L2:L1 of 1:1:1, due to limitation of the assay. Thus, the DNA ratio of 1:1:3 was used, rather than 1:1:1, in order to detect improvement in specificity. All transfections were performed using 300 ng of H1, 300 ng of L1 and 900 ng of L2 (i.e. H1:L1:L2 or H2:L2:L1=1:1:3 ratio), 100 ng of AKTdd pTT22 (a vector containing a constitutively active protein kinase B mutant), and 400 ng of ssDNA (salmon sperm DNA).

### Data Analysis and Description of LCCA Metrics

Data analysis was carried out using a modified version of the analysis described in Example 4, as set forth below.

All LCCA design sets were tested in the LCCA, and designs of interest were identified based on the following inclusion criteria. In order to be considered a design of interest, the design had to contain at least one positive LCCA result above the upper limit of 95% WT scalar confidence interval, while the other complementary LCCA had to be above the lower limit of 95% WT scalar confidence interval. All designs that passed the above criteria were included in a second K-L design library as identified in Table 7A, or a second K-K derived K-L design library as identified in Table 7B.

Table 8A and Table 8B describe the LCCA data obtained for the second K-L design library and the second K-K-derived K-L library, respectively, in the context of Mab designs. The performance of each LCCA design set in this Example was described by two scalar values that corresponded to the natural logarithm of the ratio of H1L1: H1L2 (median value) and the ratio of H2L2:H2L1 (median value), respectively. Note that these scalar values are slightly different from the scalar values calculated in Example 4, but are similarly used to assess preferential pairing. These scalar values are reported in columns 2 and 5 in Table 8A for the second K-L design library, and in Table 8B for the second K-K-derived K-L library. For the wild type CAT-2200 (HC) competed against the CAT-2200 (LC-FLAG) plus the Pertuzumab (LC-HA) the 95% scalar confidence interval was-3.24 (lower limit) to −2.94 (upper limit). For the wild type Pertuzumab (HC) competed against the Pertuzumab (LC-HA) plus the CAT-2200 (LC-FLAG) the 95% scalar confidence interval was-0.9 (lower limit) to −0.6 (upper limit).

The scalar range for each LCCA of a design (H1L1L2 and H2L1L2 experiments) is shown in columns 3 and 6 in Tables 8A and 8B as a measure of the reproducibility of the results, and represents the difference between the highest and lowest scalar values measured for the design. For results reported from a single measurement the range values are marked NA (not applicable). In addition, scalar values were also converted back into a pure median ratio for H1L1: H1L2 and H2L2:H2L1. This conversion effectively normalized the LCCA ratios to 100% as it was observed in some cases that the total amount of H1L1 and H1L2, or H2L2 and H2L1 significantly differed from 100%. These values are reported in columns 4 and 7 in Tables 8A and 8B.

### Results

As noted above, the LCCA results are shown in Tables 8A and 8B in the context of Mab design sets. Table 8A provides the LCCA data relating to the K-L designs described in Example 9. 151 K-L designs met the inclusion criteria, and most of the designs have amino acid modifications in the constant region only, with a few of the designs also incorporating amino acid modifications in the variable region (see Table 7A). These designs were proposed to further drive pairing specificity and stability while also favoring transferability to other antibody systems.

Table 8B provides the LCCA data relating to the K-K-derived K-L designs also described in Example 9. 21 K-K-derived K-L designs met the inclusion criteria. These designs include constant domain designs only.

### Example 11: Scale Up of Designed Fab Heterodimers for Biophysical Characterization

In order to examine the biophysical characteristics of correctly paired heterodimers, the preparation of selected correctly paired heterodimers H1L1 or H2L2 (designed Fabs) identified in the unique identifier sets shown in Tables 7A and 7B was scaled up as described in Example 5, with the exception that a 50 ml scale-up was performed instead of 20 ml. Expressed Fabs were purified as described in Example 5 in order to test for thermal stability and antigen binding. The purification protocol was modified to incubate supernatants with the IgG-CH1 CaptureSelect™ resin without any initial dilution in the equilibration buffer and elution of bound samples was performed with 4 bed volumes of elution buffer.

Following purification, heterodimer expression was assessed by non-reducing and reducing High Throughput Protein Express assay using Caliper LabChip GXII, as previously indicated in Example 5.

The data demonstrated that inclusion of Mab design set modifications in the designed Fabs did not have a significant impact on protein expression, judging by the post purification yield. Post-purification yield obtained for designed CAT-2200 Fabs was 1.3 mg/50 ml expression (SD of 0.6), compared with 1.9 mg/50 ml expression (SD of 0.5) for the wild type Fab. Designed pertuzumab Fabs were prepared with a yield of 0.9 mg/50 ml expression (SD of 0.4), compared with 1.1 mg/50 ml expression (SD of 0.2) for the wild type Fab. Caliper analysis showed a degree of purity comparable to WT Fab (data not shown). A number of designed Fabs had precipitate associated with them (noted with ‘*’ in Tables 9A and 9B) post purification. Yield information provided above was measured following removal of the precipitate.

### Example 12: Antigen Affinity Measurements of Designed Fab Heterodimers

The ability of CAT-2200 designed Fab heterodimers to bind to IL-17A and Pertuzumab designed Fab heterodimers to bind to HER2-ECD was assessed in order to determine whether the amino acid substitutions had any effect on the ability of the heterodimer to bind to antigen. Antigen-binding was assessed as described in Example 6, except that CAT-2200 designed Fabs were injected at 50 μL/min for 120s with a 600 to 800s dissociation phase, instead of a 900s dissociation phase. Furthermore, screening of designed pertuzumab Fabs for binding to HER2 was carried out as described in Example 6, with the following modifications: for each Fab capture, Fabs were diluted to 2.5 μg/ml instead of 2 μg/ml with an injection time of 120s instead of 240s. The lowest HER2 antigen concentration injected was 1.23 nM instead of 0.41 nM. The dissociation phase for the concentration series was 600s instead of 1200s.

Results. Antigen affinity (KD) values for designed Fab heterodimer samples are reported in Table 9A (K-L designs) and Table 9B (K-K-derived K-L designs) in the context of Mab design pairs. The KD values of H1L1 and H2L2 Fabs are included in columns 2 and 4 (KD of H1L1 CAT-2200 Fab heterodimer and H2L2 Pertuzumab Fab heterodimer, respectively), and 3 and 5 (change in KD of H1L1 or H2L2 Fab heterodimer compared to its respective wild-type). KD values were determined only for Fab heterodimer samples that exhibited a Fab heterodimer capture of at least 100 RU. The reference KD value for wild-type CAT-2200 Fab bound to IL-17A used for comparison to the designed heterodimers was 0.209 nM. This value is a median value based on multiple measurements of the wild-type CAT-2200 Fab heterodimer where the light chain contains a FLAG tag and does not differ significantly from the value reported in Example 6. Similarly, the reference KD value for wild-type pertuzumab bound to HER2 was 7.8 nM. This value is also a median value based on multiple measurements of the wild-type pertuzumab Fab heterodimer where the light chain contains an HA tag, and does not differ significantly from the value reported in Example 6. In Tables 9A and 9B, the difference in KD with respect to wild type antigen binding affinity is shown using the calculation-(log(KD_design)-log(KD_wt)), such that positive values indicate lower KD values whereas negative values indicate increased KD values of the Fab heterodimer compared with wild type binding affinity for antigen. As noted earlier, due to throughput limitations in the scale up procedure for the large number of Fab heterodimers generated by the Mab design sets, SPR measurements were performed by sampling a subset of designed Fab heterodimers. For that reason, some Fab heterodimers lack KD values measured by SPR and are hence marked as not determined (ND). For the designed Fab heterodimers tested, amino acid substitutions had little or no impact on KD values (within 2-fold of WT).

### Example 13: Thermal Stability Measurements of Designed Fab Heterodimers by DSF

Differential Scanning Fluorescence (DSF) was used to measure the thermal stability of correctly paired designed Fab heterodimers in comparison to that of wild type Fabs. Fab heterodimers were prepared as described in Example 11.

### Measurement of Thermal Stability by DSF

The thermal stability of designed Fab heterodimer was measured using DSF as follows. Each purified heterodimer was diluted to 0.67 mg/mL in DPBS (Dulbecco's phosphate buffered saline, HyClone Cat #SH30028.02). A working stock of Sypro Orange gel stain (Life Technologies Cat #S-6650) was prepared by 1:1000 dilution in DPBS. The DSF samples were prepared by adding 15 μL of 0.67 mg/mL protein to 15 μL of the Sypro Orange gel stain working stock. However, for proteins that had a concentration less than 0.67 mg/mL, each DSF sample was prepared by adding 15 μL of the undiluted protein to 15 μL of a working stock of Sypro Orange dye. DSF analysis was then conducted on 30 μl aliquots using the Rotor-Gene 6000 qPCR instrument (QiaGen Inc). Each sample was scanned from 30° C. to 94° C. using 1° C. intervals with a 10 second equilibrium between each step and a 30 second wait time at the start. An excitation filter of 470 nM and emission filter of 610 nM with a gain of 8 was used. Data was analyzed with the Rotor-Gene 6000 software using the maxima value from the first derivative of the denaturation curve as the Tm.

DSF results for designed Fab heterodimer samples are reported in Tables 9A (K-L designs) and 9B (K-K-derived K-L designs), in the context of Mab design sets. Thermal stability of H1L1 and H2L2 Fabs are included in columns 6 and 8 (H1L1 refers to CAT-2200 Fab heterodimer and H2L2 to Pertuzumab Fab heterodimer) of Tables 9A and 9B. For Fab heterodimers where repeats were conducted, the reported Tm value is the median value. Comparisons of the designed Fab heterodimer Tm values with respect to the Tm value of the wild-type Fab heterodimer are reported in column 7 for the CAT-2200 H1L1 and in column 9 for the Pertuzumab H2L2. The median Tm for wild-type CAT-2200 Fab construct containing a FLAG tag was determined to be 77.0° C. The median Tm for wild-type Pertuzumab Fab heterodimer containing a HA tag, was determined to be 81.7° C. As mentioned earlier DSF measurements were performed by sampling a subset of designed Fab heterodimers, hence some Fab heterodimers lack Tm values measured by DSF and are marked as not determined (ND). The majority of the designs tested showed good thermostability (exhibiting a Tm decrease compared to WT of up to 4-5° C.), within the range of expected Tm values commonly observed for wild-type Fabs, with the following exception. The presence of the S131K_L135K mutations in the kappa light chain in combination with L124E K145T mutations in kappa heavy chain in two Fab heterodimers appeared to negatively affect their Tm values by >10° C. Also of note, for some of the CAT-2200 designed Fabs, a shoulder to the main Fab peak was observed in the DSC spectra (these Fabs are marked with ‘#’ in Tables 9A and 9B). Such a profile was also observed for the wild-type CAT-2200 Fab.

### Example 14: Thermal Stability Measurements of Designed Fab Heterodimers by DSC

To complement the measurement of thermal stability by DSF, the more precise method of differential scanning calorimetry (DSC) was also used to measure the thermal stability of correctly paired, designed Fab heterodimers in comparison to that of wild type, unmodified heavy chain-light chain pair. Fab heterodimers were prepared as described in Example 11 and thermal stability measure by DSC as described in Example 8. However, for Fab heterodimers that had sample concentrations lower than the ones indicated in the protocol, DSC was performed at the available concentration.

Thermal stability measurements for H1L1 and H2L2 Fabs are shown in columns 10 and 12 (H1L1 refers to CAT-2200 Fab heterodimer and H2L2 to Pertuzumab Fab heterodimer, respectively) of Table 9A and 9B. For each Fab heterodimer where repeats were conducted, the reported Tm value is the median value. Comparisons of the designed Fab heterodimer Tm values with respect to the Tm value of the wild-type Fab heterodimer are reported in column 11 for the CAT-2200 H1L1 (wild type CAT-2200 Fab construct containing a FLAG tag, with a median Tm of 70.7° C.) and column 13 for the Pertuzumab H2L2 (wild type Pertuzumab Fab construct containing a HA tag, with a median Tm of 78.1° C.). Given the high requirement for sample amounts and low throughput nature of DSC, measurements were performed by sampling a subset of designs that were characterized by DSF. For that reason, some Fab heterodimers lack Tm values measured by DSC and are hence marked as not determined (ND). All of the designed Fab heterodimers assayed by DSC showed good thermostability (Tm decrease compared to WT up to 2-3 C). The DSC-derived Tm values were generally lower than those observed by DSF (Example 13). Although the absolute difference in Tm of the designed Fabs compared to WT was observed to be dependent on the method used, whether DSF or DSC, the relative ranking of the designed Fabs with respect to the wild-type Fab was the same independent of the method used.

### Example 15: Design Clustering and Selection of Representative Designs

Examples 2 to 14 described the design and testing of K-L designs and K-K-derived K-L designs. The ability of these designs to promote pairing was tested in the LCCA, in a Fab format where the ability of one heavy chain of each LCCA design set to pair with the appropriate light chain was determined. The effects of the amino acid substitutions on the stability and antigen-binding of the resulting correctly paired heterodimers (one heavy chain and one light chain) were also assessed.

In order to test whether the preferential pairing results for the Mab design sets (H1L1H2L2) obtained in LCCA format (co-expression of H1, L1, and L2, or of H2, L1, and L2) for the K-L designs and the K-K-derived K-L designs also applied when the polypeptide chains of a Mab design set were co-expressed (i.e. when H1, L1, H2 and L2 were co-expressed), a representative number of the over 400 designs described in Tables 4A, 4B, 7A, and 7B was selected, as it was not feasible to test all of the designs in this way. The representative designs were tested in an assay referred to as “SMCA” (described in detail in Example 16).

To select representative designs, the K-L designs identified in Tables 4A and 7A (n=404) were combined and clustered according to identity in the amino acid residue positions substituted. In some cases where a K-K-derived K-L design from Table 7B had been significantly modified from the original K-K design, it was also included in this K-L design group for clustering. Tables 10-A1 to 10-A12 show the resulting clusters 1 to 12 for the K-L designs. Likewise, the K-K-derived K-L designs reported in Tables 4B and 7B (n=43), excluding those moved to the K-L design group, were also combined and clustered. Tables 10-B1 to 10-B10 show the resulting clusters 1 to 10 for the K-K-derived K-L designs. Representative designs from each cluster were then selected as described below. The final set of selected representative designs is also included in Table 11A (K-L designs) and 11B (K-K derived K-L designs) including SMCA Design ID designation.

### Representative Designs for SMCA

Representative designs were selected on the basis of primary criteria of demonstrating pairing specificity and having minimal or no impact on antigen binding, as well as taking into account secondary criteria such as: design stability, minimizing for the number of substitutions, comparable expression to WT. Maximal pairing specificity was assessed taking into account both Fabs as well as each Fab individually where one Fab paired less well than the other Fab in the heterodimer pair, but still demonstrated better than the wild-type pairing (Scenarios A and B as shown in FIG. 6). In addition, the diversity within a cluster and the number of designs in a cluster further guided the type and number of cluster representatives selected.

A total of 33 representative K-L designs were selected from clusters 1 through 12 for testing in SMCA format. Likewise, 7 K-K-derived K-L designs with high to medium average LCCA performance values were selected from 7 clusters of the 10 clusters identified. Three of the K-K-derived K-L design clusters did not include designs that met the criteria of a representative with respect to primary criteria such as LCCA performance criteria or lack of effect on antigen binding.

At least one representative design was chosen from each cluster. Clusters that were represented by only one representative design included K-L design clusters 5 and 11, and all of the 7 K-K-derived K-L design clusters. The remaining clusters had more than one representative design as the clusters were either large (i.e. clusters 2, 3, 4, 6, 7, 8) and/or had more design sub-diversity (minor clusters) (i.e. clusters 1, 9, 10, 12). Although the designs within each cluster shared sequence similarities, minor clusters within a cluster differed in at least one driver (amino acid substitution). The representative designs for each cluster are highlighted in bold in Tables 10-A1 to 10-A12 and 10-B1 to 10-B10. As noted elsewhere herein, “-” in these tables indicates that no amino acid substitutions that promote preferential pairing were present in that particular polypeptide chain.

### Drivers and Secondary Substitutions

Clusters for which representatives were chosen are described here, with a focus on identifying the “drivers” or “driver sets” (amino acid positions and/or substitutions) that promoted the desired pairing specificity in each cluster, as assessed by the LCCA. The term “driver set” refers to a set of amino acid positions and substitutions that promote preferential pairing, while the term “driver” refers to a single position/substitution within a driver set. However, other residues and substitutions were sometimes associated with a design or set of designs in a cluster. These residues and substitutions are referred to as “secondary” substitutions, and were sometimes included in a Mab design set to improve the performance of the driver set or driver sets used. Mechanistically, these secondary substitutions may act to i) promote steric accommodation of drivers, ii) increase the number of contacts or substitute for contacts lost upon substitutions at driver set positions, at the interface between heavy and light chains, iii) optimize the hydrogen-bonding network based on driver sets, and/or iv) create an environment conducive to improved performance of driver sets. One or more of these mechanisms can be used/are intended to improve the performance of the drivers in each cluster.

Some of these secondary substitutions were common across several clusters. For example, the K145T substitution in the heavy chain was utilized in either H1L1 or H2L2 to remove the positive charge when a negatively charged driver or drivers were designed in its vicinity. In a number of clusters, the K129T substitution in the lambda light chain was sometimes utilized to remove the positive charge when a negatively charged driver or drivers were designed in its vicinity (e.g. K-L design cluster 4 and clusters 8-12). Furthermore, the E124Q substitution in the lambda light chain was utilized in some clusters to remove the negative charge in the vicinity of a positively charged driver or drivers (e.g. K-L design clusters 1-3). The V133G substitution was applied predominantly in the kappa light chain in some of the designs to sterically accommodate particular driver sets (e.g. predominantly in K-L design clusters 1, 3, 4, 6, 7, 10, 12 and K-K-derived K-L design cluster 10). Y178T in the lambda light chain was introduced in designs for steric accommodation of specific driver sets in the respective H1L1 (e.g. K-L clusters 4, 5, 8 and 12). V133S in the light chain was applied in yet another driver set (e.g. K-L clusters 1, 2 and 12) targeting improved hydrogen bonding network.

### Description of K-L Design Clusters

For cluster 1, two designs (13175-13486 and 13180-13515) were chosen to represent the cluster as these designs utilized similar electrostatic drivers occupying similar space (see Table 10-A1). For all members of this cluster, a combination of two driver sets was utilized. A first electrostatic driver set was designed to allow negatively charged substitution (predominantly L143D) in H1 to form salt bridges with a positively charged substitution (T131K/R) in L1, while H2 was designed to allow for a) positively charged substitutions, S188K or L124R_S186R, to form salt bridges with negatively charged substitutions in L2 (S176D_T178E or S176D_T180E) or b) formation of salt bridges between L143R or S186K in H2 and Q124E_V133D in L2. A second electrostatic driver set, termed a “disulfide steering driver set,” was added to disfavor formation of the H-L or heterodimer disulfide bond in mispaired heterodimers in the early stages of the H-L assembly process. The disulfide steering driver set used a positively charged substitution in H1 (A125R), a negatively charged substitution in L1 (S122D), a negatively charged substitution in H2 (K228D), and a positively charged substitution in L2 (S121K). These amino acid substitutions are located in the proximity of the disulfide bond, away from the buried (main) H-L interface where the majority of amino acid substitutions in the K-L designs are located. The two driver sets were designed to form salt bridges in the preferentially paired heterodimers while the mismatched pairs would be disfavored primarily due to electrostatic repulsion.

For cluster 2, four representative designs (13282-13484, 13287-13494, 13218-13482 and 10945-11377) were chosen to reflect the degree of diversity in this cluster (see Table 10-A2). All members of this cluster are based on H1 designed similarly as in the cluster 1 first electrostatic driver set, to allow negatively charged substitutions (L143D or L143D_Q179E) to form salt bridges with a positively charged substitution (T131K/R) in L1, while H2 was designed to allow for either a) positively charged substitution (S186K) to form salt bridges with negatively charged substitutions in L2, primarily V133D or V133D_Q160E; b) S188K to pair with S131D/E of L2; or c) L143R/K to pair with Q124E_V133D of L2. Some members of cluster 2 also contained a steric driver set in addition to the electrostatic driver set. Two steric driver sets were used, termed “steric 1” and “steric 2.” The steric 1 driver set included F174G in H1, designed to be sterically complementary to S176F or T116F_S176F of L1, and V190F of H2 sterically complementary to L135A of L2. The steric 2 driver set included A139W in H1 sterically compatible with WT residues of L1 and WT of H2 sterically compatible with L135W of L2. Hence in this cluster, mismatched pairing of H1L2 and H2L1 would be disfavored primarily due to electrostatic repulsion or electrostatic repulsion in combination with the steric incompatibility. There are also two cluster members that in addition to the electrostatic driver set contain a variable design component (“variable domain driver set”) in the H2 chain (Q39E or L45P) and L2 chain (Q38R or P44F, respectively) for which representatives were not chosen, but would be expected to improve pairing.

For cluster 3, five representative designs (10931-11263, 13221-13411, 10987-11257, 10983-11306 and 10947-11392) were chosen (see Table 10-A3). Members of this cluster utilized similar electrostatic driver sets in H1 (L143D or L143E_Q179E) and L1 (T131R/K) as in cluster 2, but differed with respect to the design of H2L2. The design of H2L2 was based primarily on a) L124R_S186R/K or L124R_Q179K or S188K of H2 pairing with S176D_T178E or S176D_T180E of L2; or b) L143R_S188K of H2 pairing with Q124E_V133D_S176D_T178D/E of L2, leading to disfavoring mismatched pairs primarily due to electrostatic repulsion. As in cluster 2, some members of the cluster also utilized a steric 1 or steric 2 driver set in addition to the electrostatic driver set. A representative of such a driver set combination is 13221-13411. This cluster also included two designs containing a ‘variable domain driver set’ in addition to the electrostatic driver set.

Three representative designs (13335-13361, 13209-13364 and 11100-11205) were chosen for cluster 4 (see Table 10-A4). Most members of this cluster utilized similar electrostatic drivers on H1 (S188K) and L1 (S176E/D_Y178E) with several variations in the H2L2 pair: a) V177D_S188D of H2 pairing with S176K_T178R/K of L2; or b) S186E or L124E or L124E_Q179E of H2 pairing with S176K/R or S131K/R_S176R/K of L2, which would allow for the formation of salt bridges in the preferentially paired heterodimers while the mismatched pairs would be disfavored primarily due to electrostatic repulsion. Some members of this cluster utilized only a steric driver set, i.e. a steric 3 driver set or some components of the steric 3 driver set, in addition to the electrostatic driver sets. The steric 3 driver set was based on WT residues in H1L1 or S188A in H1 and S176A_Y178W in L1 and S188W or S186I/L S188W in H2 designed to be sterically complementary to S176V or S176A T178A in L2. Furthermore, some members of this cluster also contained the disulfide steering driver set in addition to the electrostatic driver sets. In addition, a few cluster members contained the variable domain driver set(s) in addition to the electrostatic driver.

Cluster 5 was represented by unique identifier 11066-11181 (see Table 10-A5). All members of this cluster utilized electrostatic driver sets. A first set of electrostatic drivers was based on S186K in H1 pairing with V133D in L1 and S188D or V177D_S188D of H2 pairing with S131K or S176K_T178R/K respectively, in L2. A second set of drivers was of reversed charge orientation, primarily L124E_V190D in H1 pairing with L135K in L1 and L124R or S188K of H2 pairing with S176D or S176D_T178E of L2.

For cluster 6, two representative designs (10808-11308 and 10814-11233) were chosen (see Table 10-A6). All members of this cluster utilized similar electrostatic driver sets in the H1L1 pair: V177D_S188D of H1 with S176K_Y178K/R of L1 and a more diverse set of drivers in the H2L2 pair, namely a) S188K of H2 pairing with S176E/D_T178E or S131D/E of L2; b) S186K/R of H2 pairing with V133D or Q124E_Q160E_T180E of L2; c) L124R or L124R_Q179K of H2 pairing with S176D or S176D_T178D or S176D_T180E of L2; or d) L143K/R of H2 pairing with V133D or Q124E_V133D of L2. These substitutions allowed for formation of salt bridges in the preferentially paired heterodimers while the mismatched pairs would be disfavored primarily due to electrostatic repulsion.

For cluster 7, three designs (13167-13514, 13263-13493 and 12878-11240) were chosen to represent the cluster (see Table 10-A7). All cluster members utilized electrostatic driver sets based on a common driver set in H1L1: S188E in H1 and Y178K in L1, with some driver set diversity in the H2L2 pair: primarily L124R or S188K (also in combination with L143K or S186R or Q179K) of H2 pairing primarily with S176D/E_T178D/E or S176D/E_T180E or in combination with Q124E of L2. Some of the cluster members also contained the disulfide steering driver set, the steric 2 driver set or a variable domain driver set in addition to the electrostatic driver set.

For cluster 8, five designs (13320-13370, 12938-13469, 13226-13434, 11026-11270 and 133I6-13451) were chosen to represent the cluster (see Table 10-A7). Members of this cluster had more variability in the HILL electrostatic pair while the H2L2 pair was based on L143E or L143E_Q179E drivers in H2 pairing primarily with Q124R/K_T178R or Q124R_Q160R/K_T178R drivers in L2. The H1L1 driver diversity was as follows: a) S186R or Q179K utilized as drivers in H1 paired primarily with the S180E driver of L1 (the S186K variation in H1 paired with the V133D driver in L1); b) the L143K driver of H1 paired with V133D or T131E/D_V133D of L1; or c) S188K of H1 paired primarily with S176D/E Y178E of L1. Some of the cluster members contained the steric 2 or steric 3 driver set or steric 4 driver set based on L124W in H2 engineered to be sterically complementary to V133A in L2 (some cluster members as part of the latter design also utilized an L143A substitution in H1 and a V133W in L1) in addition to the electrostatic driver set. Furthermore, a few cluster members contained a variable domain driver set in addition to the electrostatic set, for which one representative was chosen, or contained an engineered disulfide bond between F122C of H2 and Q124C of L2.

For cluster 9, two representative designs (13208-13455 and 13194-13427) were chosen (see Table 10-A9). Almost all members of this cluster utilized a combination of two electrostatic driver sets. A first driver set was predominantly composed of Q179K or S186R in H1 and S180E in L1 with L143E or L143E_Q179E in H2 and S131K or Q124R_T178R or Q124R_Q160K_T178R in L2, and the second driver set was the disulfide steering driver set.

For cluster 10, two representative designs (13238-13463 and 13304-13466) were chosen (see Table 10-A10). Members of this cluster were based on steric 1 drivers alone, or in combination with the electrostatic driver sets used in cluster 9.

For cluster 11, one representative design (13306-13375) was chosen (see Table 10-A11). All members of this cluster utilized electrostatic substitutions to drive preferential pairing of heterodimers, based on the following drivers: L143K_V190K in H1 and V133D in L1 and L124E in H2 and S131K_L135K in L2.

For cluster 12, three representative designs (13227-13383, 11065-11206 and 11034-11204) were chosen (see Table 10-A12). Members of this cluster primarily utilized electrostatic substitutions to drive preferential pairing of heterodimers. These included L143K or S186K in H1 and V133D or T131D/E_V133D in L1 and predominantly L124E_Q179E in H2 and S131K/R_S176R in L2. Some cluster members additionally included the disulfide steering, steric 2, steric 3, or variable domain driver sets.

### Description of K-K-Derived K-L Design Clusters

A significantly smaller set of K-K-derived K-L designs was tested (n=43) in the LCCA compared to the K-L designs. Hence, each of the ten kappa-kappa ported clusters was represented by a single design. Most of the clusters utilized electrostatic driver sets, except for two that utilized steric driver sets. Furthermore, two clusters were composed of designs in the variable domain only, while the rest of the clusters comprised constant domain designs.

Cluster 1 included electrostatic designs based on the following set of drivers: L143E_Q179E in H1, E124K_Y178R in L1, S186R in H2, and T178E_T180E or Q160E_T180E in L2 and is represented by design 10657-10760.

Cluster 2 included electrostatic driver sets featuring L143E in H1, E124R in L1 and S186R or Q179K in H2, Q124E_Q160E_T180E in L2 (design representative 10652-10734).

Cluster 3 contained a positively charged driver S186R in H1 complementary to a negatively charged driver S180E in L1, and a negatively charged driver L143E or Q179E or their combination in H2 complementary predominantly to a positively charged driver Q124K_T178R in L2 (design representative 10685-10726).

Cluster 4 utilized the following drivers: Q179K in H1, S180E in L1 and L143E in H2, Q124R or Q124R_Q160K_T178R in L2 (design representative 10665-10724).

Cluster 6 members contained variable domain designs based on the electrostatic pair of Q39K/R and Q38E/D in one arm and Q39E/D with Q38K/R in the other arm (both charge pair orientations were utilized in H1L1 and H2L2 arms) (design representative 10681-10741).

Cluster 8 members included a second set of variable domain drivers, steric in nature: WT or L45F in H1 complementary to WT in L1, and L45A/P in H2 complementary to P44F of L2 (design representative 10621-10733).

Cluster 10 included a single design based on L124E in H1, S176R in L1 and L124R in H2 and S176D in L2 (design 10640-10713).

Representatives for clusters 5, 7 and 9 were not selected as they did not meet performance criteria, such as moderate to strong driving or lack of impact on antigen binding relative to the parental Fab.

### Example 16: Assessment of Preferential Pairing of Designed Heterodimers in Mab Design Sets in a Bispecific Antibody Format

Examples 2 to 4, and 10 demonstrated the ability of the designed Fabs to preferentially pair in the context of an LCCA design (i.e. H1, L1, L2, or H2, L1, L2, where one heavy chain was co-expressed with two light chains in a Fab format). In this example, designed heterodimers in Mab design sets (H1, L1, H2, L2) were assessed to determine if they promoted preferential pairing in a bispecific antibody format (i.e. where H1, L1, H2, and L2 are co-expressed). In addition to the amino acid substitutions of the Mab design set, the Fc region of the full-length heavy chain of each heterodimer was asymmetrically modified such that one heavy chain comprised the amino acid substitutions T350V, L351Y, F405A and Y407V (Chain A) and the other heavy chain comprised the amino acid substitutions T350V, T366L, K392L and T394W (Chain B) where the amino acid numbering of the Fc is according to the EU numbering system. These asymmetric Fc modifications were included in order to promote heterodimerization of the unique heavy chains.

The ability of designed heterodimers in Mab sets to promote preferential pairing in the context of a bispecific antibody was assessed in 3 bispecific systems using combinations of the CAT-2200 and pertuzumab antibodies described in prior examples, and a CR8071 (anti-influenza B hemagglutinin protein) antibody and SGN-CD19a (anti-CD19). The 3 bispecific systems tested were: A) CAT-2200 (having a lambda light chain)/pertuzumab (having a kappa light chain), B) CAT-2200 (having a lambda light chain)/SGN-CD19a (having a kappa light chain), and C) CR8071 (having a lambda light chain)/SGN-CD19a (having a kappa light chain). CAT-2200 and CR8071 are human antibodies, while pertuzumab and SGN-CD19a are humanized antibodies.

### Assay Format (SMCA)

The ability of heterodimers containing amino acid substitutions of a Mab design set to preferentially pair to form a correctly paired bispecific antibody was assessed as described below. The assay is based on co-expressing the four chains (H1 and L1 chains from one antibody with the H2 and L2 chains from the other antibody) and detecting the presence of correctly formed bispecific antibody using mass spectrometry (LC-MS). FIG. 8 provides a schematic depicting the four starting polypeptide chains and the potential products resulting from co-expression of these starting polypeptide chains in the absence of preferential pairing between heavy and light chains (in both Fab and Fc regions) of the heterodimer pairs. Two unique full-length heavy chain constructs were co-expressed with two unique light chain constructs, yielding ten possible antibody species (also referred to as Mab species): H1L1_H1L1, H1L2_H1L2, H1L1_H1L2, H2L1_H2L1, H2L2_H2L2, H2L1_H2L2, H1L1_H2L1, H1L2_H2L2, H1L2 H2L1 and H1L1 H2L2. The H1L1_H2L2 species is the correctly paired bispecific antibody (see FIG. 8). Four types of half-antibody (half-Ab) species are also possible, as shown in FIG. 8. When modifications are introduced into the Fc region to promote heterodimerization of the unique heavy chains, the number of potential Mab species decreases (i.e. less of species E to J are observed). The relative pairing specificity in terms of amount of correctly paired bispecific antibody species H1L1_H2L2 vs. other species was determined using LC-MS after protein A (pA) purification and deglycosylation. When possible, chains were left untagged, provided that all Mab species and half-Ab species differed from each other by at least 50 Da. When mass differences precluded this possibility, N-terminal tags (HA or FLAG) were added to the light chains in order to provide sufficient mass differentiation between species, with emphasis on the use of the FLAG tag where possible, as cleavage of HA tag was sometimes observed (in the case of constructs containing CR8071, only the FLAG tag was used).

This assay, involving the expression of H1, L1, H2, and L2 of a bispecific antibody, and subsequent analysis of paired products is referred to as SMCA.

### Preparation of Constructs

Constructs encoding the CAT-2200, pertuzumab, CR8071 and SGN-CD19a IgG heavy and light chains comprising amino acid modifications according to the designs were prepared as follows. The CAT-2200 and pertuzumab light chain sequences were prepared as described in Example 3, while the full-length heavy chain sequences for these antibodies were created by appending the IgG1*01 DNA sequence encoding the partial hinge-CH2-CH3 domains [SEQ ID NO: 6] and modified to promote heterodimerization, onto the C-terminus of the CH1 domain of the Fab heavy chains (containing a portion of the hinge and excluding the ABD2 extension used for constructs in the LCCA) preparation of which was described in Example 3. The constructs containing amino acid substitutions for the Mab design sets were generated either by gene synthesis or by site-directed mutagenesis as noted in Example 3. Of note, the canonical C-terminal heavy chain lysine residue was removed in order to prevent LC-MS signal heterogeneity due to C-terminal lysine clipping (Lawrence W. Dick Jr. et al., Biotechnol. Bioeng. (2008) 100:1132-43).

The base DNA sequences for the Fab portion of the heavy chain (SEQ ID NO:18) and the DNA sequences of the light chain (SEQ ID NO: 19) of CR8071 are shown in Table 3C. The CR8071 Fab amino acid sequences for heavy chain (SEQ ID NO:14) and light chain (SEQ ID NO: 15) correspond to those in the PDB entry 4FQJ (complex of Fab CR8071 and hemagglutinin) where an R222K substitution in the heavy chain was made to convert to the most common IGHG1*01 sequence.

The base DNA sequences for the Fab portion of the heavy chain (SEQ ID NO:16) and the DNA sequences of the light chain (SEQ ID NO:17) of SGN-CD19a are also shown in Table 3C. The SGN-CD19a sequences correspond to SEQ ID NOs: 7 and 17 reported in U.S. Pat. No. 8,242,252 and are included here as SEQ ID NO:12 (heavy chain) and SEQ ID NO:13 (light chain).

As noted above, the CAT-2200, CR8071, pertuzumab and SGN-CD19a light chain sequences in some of the designed heterodimers in Mab sets were prepared with or without FLAG or HA tags in order to provide sufficient mass differentiation between species. Light chains with tags were prepared as described in Example 3.

Wild-type versions of these constructs were also prepared in order to assess natural bias in each system, discern potential effect of the HA or FLAG tags on pairing and for determining the effect of designed constructs on preferential pairing over wild-type.

### Transfection, Expression and Purification of Constructs

Constructs encoding the two heavy chains and two light chains of each bispecific antibody system, either wild-type or with amino acid substitutions corresponding to each Mab design set tested were transfected into CHO-3E7 cells as follows. CHO-3E7 cells, at a density of 1.7-2×10⁶ cells/ml, were cultured at 37° C. in FreeStyle™ F17 medium (Invitrogen cat #A-1383501) supplemented with 4 mM glutamine and 0.1% Koliphor P188 (Sigma #K4894). A total volume of 200 ml was transfected with a total of 200 μg DNA with varying ratios of H1:H2:L1:L2 (which consists of 100 μg of antibody DNA and 100 μg of GFP/AKT/stuffer DNA) using PEI-max (Polysciences Cat #24765-2) at a DNA: PEI ratio of 1:4. Twenty-four hours after the addition of the DNA-PEI mixture, 0.5 mM Valproic acid (final concentration) and 1% w/v Tryptone N1 (final concentration) were added to the cells which were then transferred to 32° C. and incubated for 7 days prior to harvesting. Culture media was harvested by centrifugation and vacuum filtered using a Stericup 0.22 μm filter (Millipore Cat #SCGPU05RE). The filtered culture media was then purified using protein A MabSelect SuRe resin (GE Healthcare #17-5438-02) that was previously equilibrated with PBS pH 7.4. The antibody species bound to the resin was then washed with PBS pH 7.4 and eluted with 100 mM sodium citrate buffer pH 3.6. Eluted antibody species were concentrated and buffer exchanged in PBS pH 7.4 by centrifugation using Amicon ultra 15 centrifuge filter Ultracel 10K (Millipore #SCGP00525). The resulting protein A-purified SMCA samples containing the antibody species were assessed by Caliper prior to deglycosylation and LC-MS.

### Mass Spectrometry Method

The degree of preferential pairing of heterodimers driven by the Mab design sets in the context of a bispecific antibody was assessed using mass spectrometry after protein A purification and non-denaturating deglycosylation. As the heterodimers contained Fc N-linked glycans only, the SMCA samples were treated with only one enzyme, N-glycosidase F (PNGase-F). The purified samples were de-glycosylated with PNGaseF as follows: 0.2U PNGaseF/μg of antibody in 50 mM Tris™-HCl pH 7.0, overnight incubation at 37° C., final protein concentration of 0.5 mg/mL. After deglycosylation, the samples were stored at 4° C. prior to LC-MS analysis.

The deglycosylated protein samples were analyzed by intact LC-MS using an Agilent 1100 HPLC system coupled to an LTQ-Orbitrap XL mass spectrometer (ThermoFisher Scientific) via an Ion Max electrospray source (ThermoFisher). The samples (5 μg) were injected onto a 2.1×30 mm Poros R2 reverse phase column (Applied Biosystems) and resolved using the following gradient conditions: 0-3 min: 20% solvent B; 3-6 min: 20-90% solvent B; 6-7 min: 90-20% Solvent B; 7-9 min: 20% solvent B. Solvent A was degassed 0.1% formic acid aq. and solvent B was degassed acetonitrile. The flow rate was 3 mL/min. The flow was split post-column to direct 100 μL into the electrospray interface. The column was heated to 82.5° C. and solvents were heated pre-column to 80° C. to improve protein peak shape. The LTQ-Orbitrap XL was calibrated using ThermoFisher Scientific's LTQ Positive Ion ESI calibration solution (caffeine, MRFA and Ultramark 1621), and tuned using a 10 mg/mL solutions of CsI. The cone voltage (source fragmentation setting) was 48 V, the FT resolution was 7,500 and the scan range was m/z 400-4,000. The LTQ-Orbitrap XL was tuned for optimal detection of larger proteins (>50 kDa).

The ranges containing the multiply charged ions from the full-sized antibodies (m/z 2000-3800) and the half-antibodies (m/z 1400-2000) were separately deconvoluted into molecular weight profiles using MaxEnt 1 module of MassLynx, the instrument control and data analysis software (Waters). Briefly, the raw protein LC-MS data were first opened in QualBrower, the spectrum viewing module of Xcalibur (Thermo Scientific) and converted to be compatible with MassLynx using Databridge, a file conversion program provided by Waters. The converted protein spectra were viewed in the Spectrum module of MassLynx and deconvoluted using MaxEnt 1. The amount of each antibody species in each sample was determined directly from the resulting molecular weight profiles.

### Analysis of LC-MS Results

Overall, in most cases, the deglycosylation treatments resulted in the ability to identify all of the possible different antibody species identified by LC-MS. In many cases, each antibody species was represented by a single LC-MS peak. Exceptions included side peaks that likely also correspond to the desired bispecific species (possibly adducts or heterogeneity in the cleavage of leader peptides); however, because identity of the species resulting in the side peaks was not clear, these side peaks were not considered in the contributions to the bispecific species. Furthermore, the desired bispecific species, H1L1_H2L2, cannot generally be distinguished experimentally from the mispaired type, H1L2_H2L1, on the basis of LC-MS. As such, when bispecific antibody content is reported in the tables, it cannot be completely excluded that it does not contain this type of mispaired species. However, the very low content observed for species such as H1L2_H1L2 and H2L1_H2L1 as well as H1L2 and H2L1 half antibodies is indicative that only minor, if any, contamination of the bispecific species with mispaired species occurred.

### Assessment of Natural Bias and Effect of Tags on Pairing in Wild-Type Systems

As a first step, for each of the three bispecific systems tested, the wild-type (one of the light chains containing a tag if required for sufficient mass differentiation between species), unmodified heavy and light chains of the parent antibodies for each system were co-expressed using varying DNA ratios for H1:H2:L1:L2, and the resulting antibody species were identified and quantitated. This allowed for the identification of any inherent pairing bias (or that introduced by the presence of a tag) in each system (for example, if the light chain of one parent antibody in the bispecific system preferentially binds the heavy chain of the other parent antibody in the bispecific system), and also allowed for the identification of a DNA ratio for H1:H2:L1:L2 that provided the highest amount of bispecific antibody with the lowest amount of half-Abs. The DNA ratios were chosen to compensate for natural differences in expression levels and/or intrinsic pairing biases between heavy and light chains of the two parent antibodies and/or tag influence for each system. In tested wild-type bispecific systems A and B, L2 was tagged with a FLAG tag. In bispecific system C, neither of the light chains were tagged.

Table 12 shows the result of this assessment. The DNA ratio for H1:H2:L1:L2 that provided the highest amount of bispecific antibody with the lowest amount of half-Abs was 10:20:24:46 for System A, 15:15:35:35 for System B, and 15:15:35:35 for System C. Of note, in the CAT-2200/pertuzumab bispecific Ab system (System A), a bias was observed where the pertuzumab heavy chain (H2) preferentially paired with the CAT-2200 light chain (L1). This bias was evident when data for equivalent ratios of heavy chain 1 and heavy chain 2, but inverse ratios of light chain 1 and light chain 2 are considered (e.g. consider inverse L1: L2 DNA ratios in H1:H2:L1:L2 of 8:22:17:53 and 8:22:53:17 in Table 12). Under these conditions of H1:H2:L1:L2=8:22:53:17, prevalent mispaired Mab species of H1L1_H2L1 are much more abundant (79.6%) than in the case of H1:H2:L1:L2=8:22:17:53, where prevalent mispaired species of H1L2_H2L2 made up about 38.4%. This indicated that the pertuzumab heavy chain appeared to pair preferentially with the CAT-2200 light chain in this system, when the pertuzumab light chain had a FLAG tag.

Design SMCA constructs that contained N-terminal tags (HA or FLAG) on one of the light chains guided preparation of the analogous WT SMCA constructs that were investigated for possible influence of tags on the ability of the light chains to pair with the heavy chains. Table 13 shows the results of this experiment, and indicates that in some cases, the tag did have an impact on pairing of the light chains with heavy chains. For example, with respect to the System A case noted above, where there was preferential pairing of the pertuzumab heavy chain with the CAT-2200 light chain when the pertuzumab light chain contained the FLAG tag, when the CAT-2200 light chain contained the HA tag and the 4 polypeptide chains were expressed at the same DNA ratio, preferential pairing of the CAT-2200 heavy chain to the pertuzumab light chain was observed (see Table 13, ‘System A, L1-HA’ and compare ‘System A, L1-HA’ with ‘System A, L2-FLAG’ at the same ratio of 10:20:24:46). Furthermore, for the SGN-CD19a/CAT-2200 bispecific system (System B), the degree of mispairing between the heavy chain of SGN-CD19a with the CAT-2200 light chain in the system that contains FLAG-tagged SGN-CD19a light chain was greater than in the other two variations of the same system at the same ratio (Table 13, ‘System B, L1-FLAG’ and contrast ‘System B, L1-FLAG with ‘System B, L2-FLAG and L2-HA’ at the same ratio of 15:15:35:35). Note that Table 13 reflects data accumulated for all repeats performed, hence species percentage may in some cases differ from the data at the initial assessment stage, provided in Table 12.

For testing each of the 40 representative designs within each bispecific system, the H1:H2:L1:L2 DNA ratios used were the ratios from the corresponding wild-type bispecific systems that yielded the highest amount of bispecific Ab species and the lowest amount of half-Ab. As indicated above, for the CAT-2200/pertuzumab system, the ratio used was 10:20:24:46 (H1:H2:L1:L2), where H1 and L1 refer to CAT-2200 and H2 and L2 refer to pertuzumab. For the SGN-CD19a/CAT-2200 and SGN-CD19a/CR8071 systems, the ratio used was 15:15:35:35 (H1:H2:L1:L2).

### Preferential Pairing Results in Bispecific Antibody Format

The results of the SMCA with the 40 representative designs tested are shown in Tables 14 to 17. Tables 14 to 17 refer to Mab design sets as “designs” and have been identified with a 4-digit number. Each 4-digit number corresponds to a Mab design set unique identifier (LCCA set unique identifiers) in Tables 10-A1 to A12 and 10-B1 to 10-B10. Table 24 provides a correspondence table between the designs tested in SMCA and the LCCA set unique identifiers. The analysis of preferential pairing was performed based on two types of calculations. The first type of calculation is noted as “% H1L1 and % H2L2 Pairing” in the Tables, and represents the amount of correct pairing between H1 and L1 and between H2 and L2 across all Mab species and half-Ab species observed, including Mab species that may have had only one correctly paired arm, as a percentage of total product (H1L1_H2L2_and_H1L2 H2L1+H1L1 H1L1+H2L2 H2L2+H1L1+H2L2+0.5×(H1L1_H2L1+H1L1 H1L2+H1L2 H2L2+H2L1_H2L2)). This calculation is referred to as the ‘total pairing’ calculation. The second type of calculation is noted as “H1L1_H2L2 and H1L2 H2L1**” in the Tables, and represents the amount of correct paired bispecific antibody as a percentage of all the Mab species (i.e. Mab species A-J in FIG. 8) observed (H1L1_H2L2_and_H1L2 H2L1/(H1L1_H2L2_and_H1L2 H2L1+H1L1 H1L1+H2L2 H2L2+H1L1 H2L1+H1L2 H2L2+H1L1 H1L2+H2L1 H2L2+H1L2 H1L2+H2L1_H2L1)). This calculation is referred to as the ‘total bispecific’ calculation. Half antibodies were not taken into account in this calculation, since if present, may be removed/minimized by preparative SEC or through further H1:H2:L1:L2 DNA titrations. Table 12 demonstrates that DNA titrations are effective in manipulating the percentage of half-Ab species expressed. The effectiveness of prep-SEC purification in reducing the amount of half-Ab of type ‘Fc Chain A’ is demonstrated in Example 18.

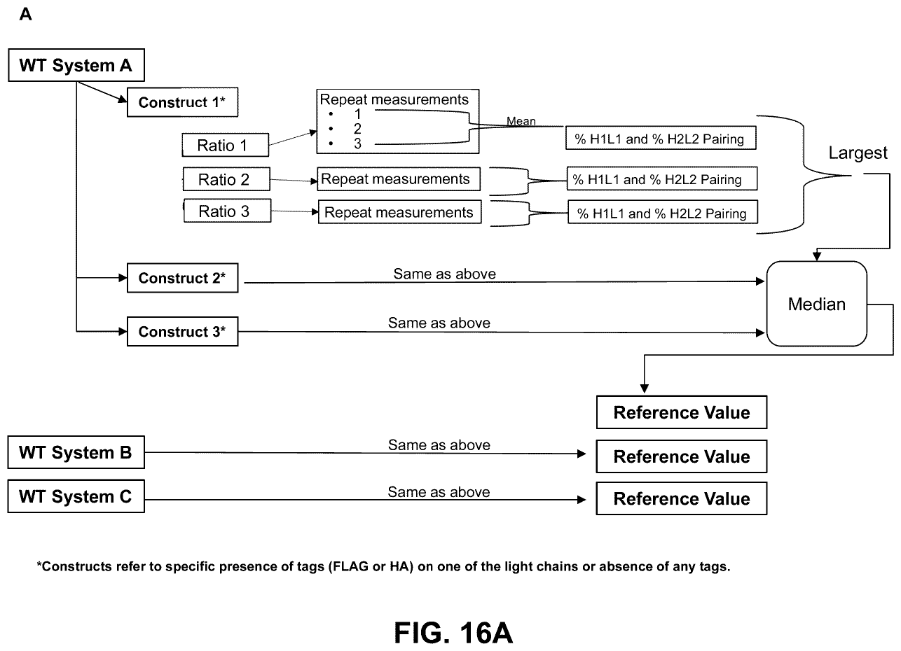

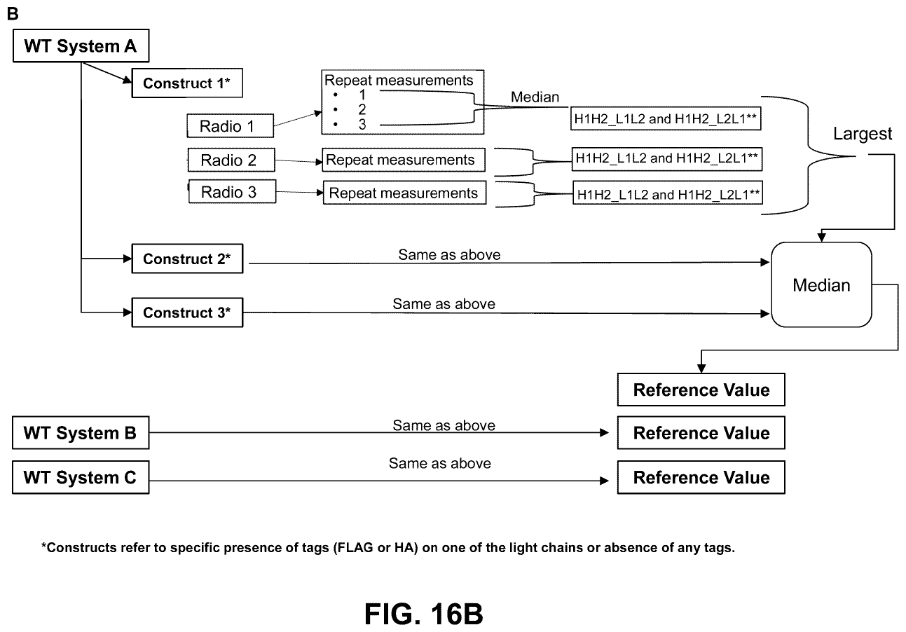

To account for biases that were observed in some wild-type bispecific systems, as described above, the preferential pairing data was reported as a comparison to the corresponding wild-type bispecific constructs at the same H1:H2:L1:L2 DNA ratio. This comparison was reported in the columns “Change in % H1L1 and % H2L2 Pairing with respect to wild-type” (i.e. change in total pairing with respect to WT) and “Change in H1L1_H2L2 and H1L2 H2L1** with respect to wild-type” (i.e. change in total bispecific with respect to WT). Where the corresponding wild-type bispecific construct had not been assessed by SMCA, a comparison was made to a similar wild-type construct. These cases are indicated by a “‡” next to the values reported. The similar wild-type construct chosen for comparison was selected as follows. For each bispecific system and construct (with or without tags), median values for “H1L1_H2L2 and H1L2_H2L1**” and the mean values for “% H1L1 and % H2L2 Pairing” from repeat SMCA experiments per DNA ratio were taken. The largest values from any ratio within each construct were taken, and the median across all constructs was used to represent the WT reference for particular system (see FIGS. 16A and 16B).

Tables 14 and 15 provide a summary of the SMCA results for the representative K-L designs tested, where designs were binned according to “Change in % H1L1 and % H2L2 Pairing with respect to wild-type” (Table 14) or “Change in H1L1_H2L2 and H1L2_H2L1′ with respect to wild-type” (Table 15). Similarly, Tables 16 and 17 provide a summary of the SMCA results for the representative K-K-derived K-L designs tested, where designs were binned according to change in total pairing (Table 16) or change in total bispecific (Table 17) calculations.

### Performance

The performance, or ability to promote preferential pairing of correct heavy and light chains, for each of the representative designs was assessed using the total pairing calculation and using the total bispecific calculation. First, a positive value in the column “Change in % H1L1 and % H2L2 Pairing with respect to wild-type” (change in total pairing with respect to WT) indicated that the Mab design set was able to promote preferential pairing. Second, a positive value in the column “Change in H1L1_H2L2 and H1L2_H2L1** with respect to wild-type” (change in total bispecific with respect to WT), was also indicative of a Mab design set that was able to promote preferential pairing.

All of the K-L designs and 18 out of 21 K-K-derived K-L designs showed reduced amounts of the primary mispaired species (H1L2_H2L2 or H1L1_H2L1) compared to wild-type although in some cases the amount of a different mispaired species was increased compared to wild-type. On average, both K-L and K-K-derived K-L designs generated the greatest increase of desired bispecific relative to wild-type (change in total bispecific with respect to WT) in system A (48.8%), followed by system B (31.1%), and system C (14.5%). On average, all of the designs tested resulted in a relatively minor increase in the total amount of half antibody compared to wild-type, from 24.1% to 27.9%, but improved the average ratio of paired to mispaired half antibody from 1.5 to 6.9.

### Transferability

The ability of the representative set of Mab designs to promote preferential pairing in multiple systems was tested as a measure of the “transferability” of the designs. Using the ‘change in total pairing with respect to WT’ calculation, 29 out of 33 K-L designs were transferable in 3 of 3 tested systems and 3 designs were transferable in 2 of the 3 systems tested (Table 14). In other words, these designs showed a positive value of ‘change in pairing with respect to WT″. When preferential pairing was measured using the ‘change in total bispecific with respect to WT’ calculation, 24 out of 33 K-L designs were identified as transferable in 3 of 3 systems and 8 designs were transferable in 2 of 3 systems (Table 15). For the K-K-derived K-L designs, 2 out of 7 designs were transferable in 3 of 3 systems and 4 designs were transferable in 2 of 3 systems using the ‘change in total pairing with respect to WT’ calculation (Table 16). Using the ‘change in total bispecific with respect to WT’ calculation, 1 out of 7 designs was transferable in 3 of 3 systems and 5 designs were transferable in 2 of 3 systems (Table 17). 23 out of 33 K-L designs and 1 out of 7 K-K-derived K-L designs were transferable in 3 of 3 tested systems when assessed using both calculations.

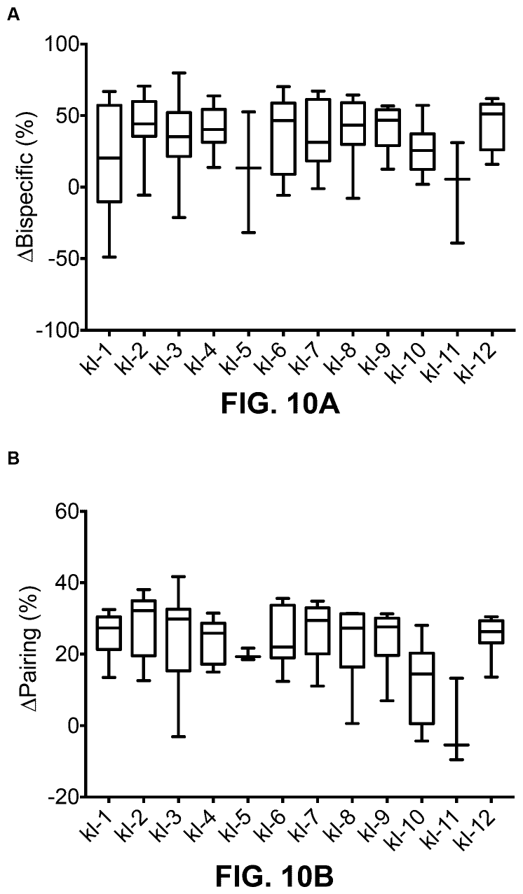

With respect to performance of the clusters, all designs within 9 of 12 K-L clusters were transferable in 3/3 systems according to the ‘change in total pairing with respect to WT’ calculation, and 4 of 12 according to the ‘change in total bispecific with respect to WT’ calculation. All designs within clusters kl-4, kl-9, and kl-12 were transferable in 3/3 systems using both calculations. FIG. 10 shows box plots comparing the performance of the representative designs from each cluster, based on the ‘change in total bispecific with respect to WT’ calculation (FIG. 10A, change in bispecific %) or on the ‘change in total pairing with respect to WT’ calculation (FIG. 10B, change in pairing %). For the K-K-derived K-L clusters, only one design per cluster was tested and so cluster transferability was equivalent to the design transferability discussed above.

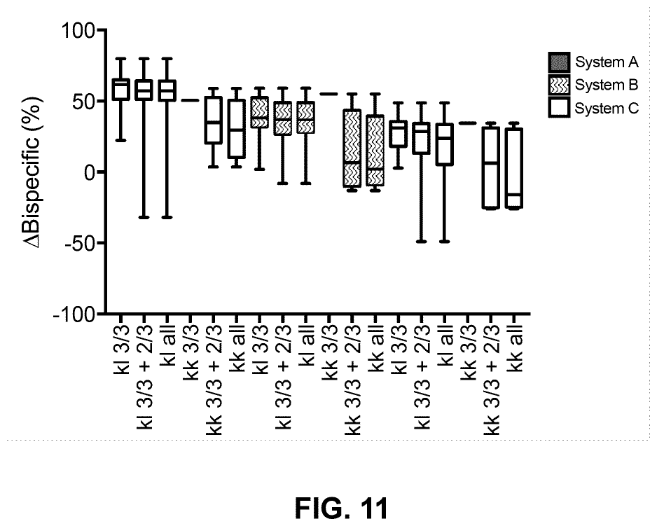

Overall, the K-L designs were able to promote preferential pairing more effectively than the K-K-derived K-L designs. FIG. 11 shows box plots depicting the ability of K-L designs and K-K-derived K-L designs to promote preferential pairing in each system tested, based on the ‘change in total bispecific with respect to WT’ calculation. For example, FIG. 11 shows that K-L designs generally promoted a larger increase in amount of bispecific antibody compared to WT than did the K-K-derived K-L designs (comparing the “kl all” value to the “kk all” value for each system). This is also true when considering only designs transferable in 2 or more or in all 3 systems.

### Example 17: Preparative Size Exclusion Chromatography (SEC) of Selected SMCA Bispecific Antibodies and Parental Mabs for Biophysical Characterization

To assess the biophysical characteristics of the bispecific antibodies generated in the SMCA described in Example 16, the protein A-purified SMCA samples from a subset of the representative designs tested were subjected to preparative SEC to remove half-Ab species. SMCA samples having at least 60% “H1L1_H2L2 and H1L2_H2L1**” (total bispecific) were selected for this step (36 in total). Some SMCA samples not meeting this cutoff were also included for completeness. For example, in cases where the SMCA samples for a given design met this cutoff in two of the three bispecific systems tested, the SMCA sample met this cutoff, but not the third, the SMCA samples for all 3 systems were included. Preparative SEC was carried out as follows. Antibody species in the SMCA samples were separated using a Superdex 200 10/300 Increase (GE Healthcare) column mounted on a GE Healthcare ÄKTA Avant 25 system equipped with an ALIAS Bio Cool autosampler (Spark-Holland) used to inject samples on the column. SMCA samples (0.9 ml) in PBS pH 7.4 (Hyclone DPBS/modified, No Calcium, No Magnesium, Cat. No. SH-300028.02) were automatically loaded into a 2 ml loop filled with PBS. Samples were then automatically injected onto the column and resolved at 0.5 ml/min with a 1 CV elution volume. Protein elution was monitored at OD₂₈₀ and collected in 0.5 ml fractions. For each SMCA sample, fractions that comprised the main peak (fractions were assessed by Caliper) were pooled and further biophysically characterized as described in Examples 19 and 20.

### Example 18: Removal of Half-Antibodies by Preparative Size Exclusion Chromatography

In Example 16, it was noted that half-Abs were excluded from calculations of pairing according to the ‘total bispecific’ calculation, since the percentage of half-Abs produced can either be manipulated by DNA titrations or, in the case of half-Abs of type ‘Fc Chain A,’ that these can also be removed/minimized by preparative SEC purification of Protein-A purified SMCA samples. To demonstrate removal of “Fc Chain A half-Abs,” purified SMCA samples from Example 17 for two K-L designs in all three bispecific systems were subjected to LC-MS analysis as described in Example 16. The two K-L designs selected were 2901 and 3972 (as identified in Table 11A). The results are shown in Table 18.

Comparing the LC-MS data in Table 18 to the respective LC-MS data for protein-A purified SMCA samples in Table 14 demonstrates that preparative SEC can be effective in the removal/minimization of half-Ab species of the type ‘Fc Chain A’ (H1L1 in 2901 and 3972). In all of the samples tested, there was enrichment in the percentage of the desired bispecific antibody species (H1L1_H2L2 and H1L2_H2L1) as well as a decrease in the percentage of half antibody species of type ‘Fc Chain A’ (H1L1 in Table 18).

### Example 19: Thermal Stability of SMCA Bispecific Antibodies

Following preparative SEC, the thermal stability of the prep-SEC purified SMCA bispecific antibodies was measured and compared to parental CAT-2200, pertuzumab, CR8071 and SGN-CD19a monoclonal antibodies in order to determine whether the amino acid substitutions had any effects on thermal stability.

### Measurement of Thermal Stability

The thermal stability of selected bispecific heterodimeric antibodies and wild-type controls was measured using differential scanning calorimetry (DSC) as follows. Following preparative SEC treatment, 400 μL samples primarily at concentrations of 0.4 mg/mL in PBS were used for DSC analysis with a VP-Capillary DSC (GE Healthcare). At the start of each DSC run, 5 buffer blank injections were performed to stabilize the baseline, and a buffer injection was placed before each sample injection for referencing. Each sample was scanned from 20 to 100° C. at a 60° C./hr rate, with low feedback, 8 sec filter, 3 min preTstat, and 70 psi nitrogen pressure. The resulting thermograms were referenced and analyzed using Origin 7 software.

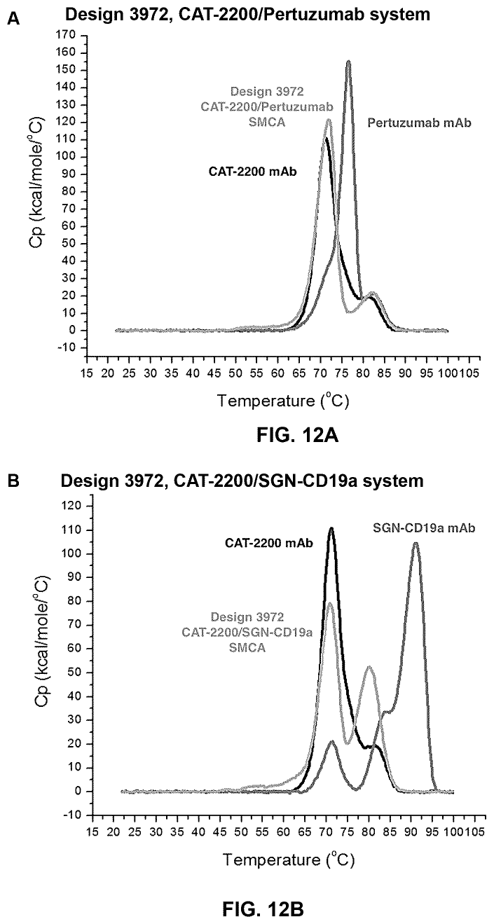

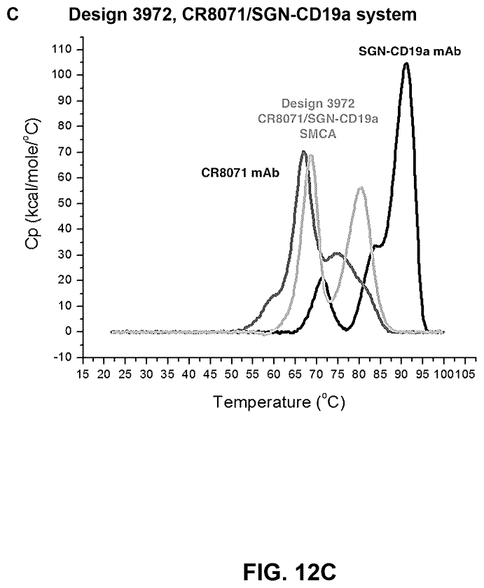

The results are shown in Table 20 and Table 21. The wild-type Fab Tm values used to calculate “Average difference in Fab Tm from wild-type (° C.)” as reported in the tables were obtained from DSC thermograms of parental antibodies of CAT-2200 (71.2° C.), pertuzumab (76.7° C.), CR8071 (66.9° C., corresponding to the primary peak) and SGN-CD19a (91.0° C.). The CR8071 parental antibody displayed two Fab transitions. For parental antibodies, additional thermal transitions were observed for the CH2 and CH3 domains of the Fc (at approximately 71° C. and 82° C., respectively) where these did not overlap with the Fab transition (FIGS. 12A-12C). Some designs with large amounts of H2L2 ‘Fc Chain B’ half antibody displayed an additional thermal transition at approximately 60° C., likely due to the presence of non-covalent homodimers. In some cases, designs caused a reduction in the Tm of the pertuzumab Fab such that it led to overlap of the CAT-2200 Fab, pertuzumab Fab, and CH2 peaks in those samples (FIG. 12A, representative design 3972). Likewise, many designs reduced the Tm of SGN-CD19a Fab to such an extent that caused overlap with the CH3 peaks in those antibodies (FIGS. 12B and 12C, representative design 3972). These overlaps led to some ambiguity in Tm assignment of the contributing Fab components, and hence the Tm values reported in Tables 20 and 21 are considered approximations. Interestingly, CR8071/SGN-CD19a bispecific antibodies containing designs displayed a single CR8071 Fab transition instead of two transitions observed in the wild-type bispecific antibodies (e.g. FIG. 12C).

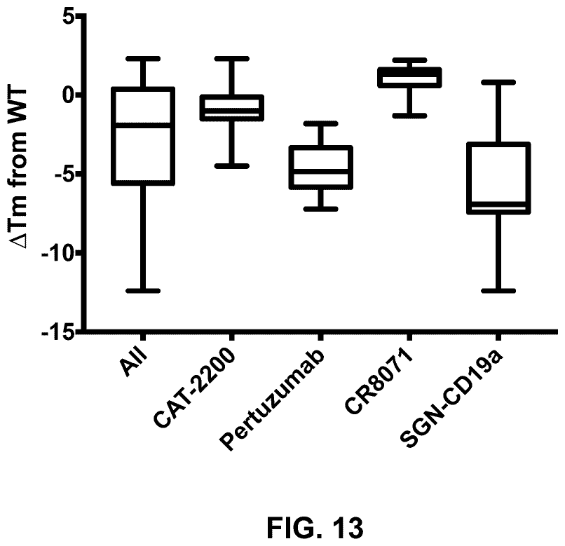

Out of all the tested K-L designs, the average difference in Fab Tm from wild-type across all systems was between 0° C. and −1.5° C. for 6 designs, between −1.5° C. and −3.0° C. for 11 designs, and between −3.0° C. and −4.5° C. for 15 designs (Table 20). Out of all tested K-K-derived K-L designs, the average difference was between 0° C. and −1.5° C. for 2 designs, between −1.5° C. and −3.0° C. for 1 design, and between −3.0° C. and −4.5° C. for 1 design (Table 21). The average differences in Fab Tm from wild-type across tested designs were −0.9° C. for CAT-2200 (STDEV=1.4), −4.6° C. for pertuzumab (STDEV=1.6), −5.8° C. for SGN-CD19a (STDEV=3.5), and +1.1° C. for CR8071 (STDEV=0.8). FIG. 13 depicts a box plot of the change in Fab Tm compared to parental antibody for the tested designs, for all designs tested (“All”), or by paratope (CAT-2200, pertuzumab, CR8071, or SGN-CD19a).

### Example 20: Antigen Affinity Measurements of Bispecific Antibodies

The ability of the bispecific antibodies to bind the appropriate antigens was assessed in order to determine whether the amino acid substitutions had any effects on antigen binding. The antigen binding affinity was determined by SPR as follows.

### SPR Biosensor Assays

For studies on Biacore™ T200: CM5 Series S sensor chip, Biacore™ amine coupling kit (NHS, EDC and 1 M ethanolamine), and 10 mM sodium acetate buffers were purchased from GE Healthcare Life Science (Mississauga, ON, Canada). For studies on Biorad ProteOn: GLC sensorchips, the Biorad ProteOn amine coupling kit (EDC, sNHS and ethanolamine), and 10 mM sodium acetate buffers were purchased from Bio-Rad Laboratories (Canada) Ltd. (Mississauga, ON). PBS running buffer with 0.05% Tween20 (PBST) was purchased from Teknova Inc. (Hollister, CA). Goat polyclonal anti-human Fc antibody was purchased from Jackson Immuno Research Laboratories Inc. (West Grove, PA). Antigens: His-tagged recombinant human CD-19 was purchased from Abcam (Cambridge, UK) and His-tagged Influenza B Hemagglutinin (B/Brisbane/60/2008) was purchased from Sino Biological (Beijing, China). Recombinant Her2 extracellular domain (ECD) protein was purchased from eBioscience (San Diego, CA). Recombinant human IL-17A was purchased from R&D Systems (Minneapolis, MN).

Surface plasmon resonance (SPR) assays with antigens Hemagglutinin and CD-19 were carried out using a Biacore™ T200 instrument (GE Healthcare) with PBST running buffer (with 0.5 M EDTA stock solution added to 3.4 mM final concentration) at a temperature of 25° C. Surface plasmon resonance assays with antigens HER2 ECD and IL-17 were carried out using BioRad ProteOn XPR36 instrument (Bio-Rad Laboratories (Canada) Ltd. (Mississauga, ON)) with PBST running buffer at a temperature of 25C.

SGN-CD19a: CD-19 affinity determination was carried out as follows. The screening of antibodies (preparative SEC purified SMCA samples and OAA controls) for binding to CD-19 antigen occurred in two steps: an indirect capture of His-tagged CD-19 onto the anti-His IgG surface, followed by the injection of five concentrations of purified antibody for kinetic analysis using single cycle kinetics. The anti-His capture surface was prepared on a CM5 Series S sensor chip by amine coupling approximately 12000 RUs of anti-His IgG (His Capture Kit, GE Healthcare) onto the active and reference flow cells, according to the manufacturer's instructions. CD-19 was injected at 10 μg/ml over the active flow cell for 60 s at a flow rate of 10 μl/min. In general, this resulted in the capture of approximately 250 RUs of CD-19 onto the anti-His IgG surface. CD-19 was not captured on the reference (blank) flow cell. The capture step was followed by five concentrations of purified antibody (200 nM and 2-fold dilutions, 80 nM and 2-fold dilutions, or 40 nM and 2-fold dilutions; depending on the antibody) that were sequentially injected over both the active and reference flow cells at 40 μl/min for 180 s with a dissociation phase of 600 s. The captured antibody surfaces were regenerated by 10 mM glycine pH 1.5 for 120 s at 30 μl/min to prepare the surfaces for the next injection cycle. At least two mock-buffer injections were performed for each analyte injection and used for referencing. The resulting single cycle kinetics sensorgrams were analyzed using Biacore™ T200 BiaEvaluation software version 3.0 and fit to the 1:1 binding model.

CR8071: Hemagglutinin affinity determination was carried out as follows. Hemagglutinin (HA) was diluted in 10 mM acetate buffer pH 5.5 and directly immobilized via amine coupling onto a CM5 Series S sensor chip. This resulted in approximately 120-130 RUs of immobilized HA. The reference flow cell was left empty (ethanolamine blocked) to use as a blank control. Antibodies were injected over the HA surface for kinetic analysis using single cycle kinetics. Five concentrations (40 nM and 2-fold dilutions) of purified antibody (preparative SEC purified SMCA samples and OAA controls) were sequentially injected over both the active and reference flow cells at 50 μl/min for 300 s with a dissociation phase of 3600 s. HA surfaces were regenerated using 2 cycles of 10 mM glycine pH 1.5 for 120 s at 30 μl/min to prepare the surfaces for the next injection cycle. At least two mock-buffer injections were performed for each analyte injection to be used for referencing. The resulting single cycle kinetics sensorgrams were analyzed using Biacore™ T200 BiaEvaluation software version 3.0 and fit to the 1:1 binding model.

CAT-2200: IL-17 affinity determination was carried out as previously described in Example 6 with the following modifications: the antibodies tested were preparative SEC purified SMCA samples and OAA controls instead of CAT-2200 Fabs. One of the control antibodies 13612 was injected at starting at 10 nM instead of 60 nM.

Pertuzumab: HER2 affinity determination was carried out as follows. All lines were immobilized horizontally with 25 g/ml Goat Anti-Human IgG, Fc□ Fragment specific (anti-human Fc). An average of 4058 resonance units (RUs) were immobilized. The anti-human Fc antibody capture surface was generated using a GLC sensorchip activated by a 1:10 dilution of the standard BioRad sNHS/EDC solutions injected for 140 s at 100 μl/min in the analyte (horizontal) direction. Immediately after activation, a 25 μg/ml solution of anti-human Fc antibody in 10 mMNaOAc pH4.5 was injected in the analyte (horizontal) direction at a flow rate of 25 μl/min for 240 s until approximately 4000 resonance units (RUs) were immobilized. Remaining active groups were quenched by a 140s injections of 1M ethanolamine at 30 μl/min for 300s also in the analyte direction to ensure mock-activated interspots are created for blank referencing. The screening of the antibodies for binding to HER2 occurred in two steps: an indirect capture of the antibodies (preparative SEC purified SMCA samples and OAA controls) onto the anti-human IgG (Fc□□Fragment specific) surface in the ligand direction followed by the simultaneous injection of 5 concentrations of purified HER2 ECD and one buffer blank for double referencing in the analyte direction. Firstly, one buffer injection for 30 s at 100 μl/min in the ligand direction was used to stabilize the baseline. For each antibody capture, antibodies were diluted to 2 μg/ml in PBST. One to five antibodies or controls were simultaneously injected in individual ligand channels for 480 s at flow 25 μl/min. This resulted in a capture of approximately 800-1200 RUs onto the anti-human Fc surface. The first ligand channel was left empty to use as a blank control if required. This capture step was immediately followed by one buffer injection in the analyte direction to stabilize the baseline, and then 100 nM, 33.3 nM, 11.1 nM, 3.7 nM and 0.41 nM HER2 along with a buffer blank were simultaneously injected at 50 μl/min for 120 s with a 300 s dissociation phase. The captured antibody surfaces were regenerated by two 18 s pulse of 0.85% phosphoric acid at 100 μl/min to prepare for the next injection cycle. Sensorgrams were aligned and double-referenced using the buffer blank injection and interspots, and the resulting sensorgrams were analyzed using ProteOn Manager software v3.1. The double-referenced sensorgrams were fit to the Langmuir binding model.

Antigen affinities of the heterodimeric antibodies were assessed with reference to the respective OAA (one-armed) wild-type controls. Antigen affinities were also obtained for the wild-type bispecific antibodies; however, SPR capture of the WT bispecifics can be heterogeneous (e.g. involving capture of mispaired heterodimers), thereby interfering with KD determination. Furthermore, wild-type affinities were measured in OAA formats containing relevant light chain tags present in antibodies. The presence of tags was observed to reduce affinity of SGN-CD-19a for CD19 by as much as 4-5 fold (Table 19). For example, the measured binding affinity of SGN-CD-19a for CD19 decreased from 67.5 nM to 224.5 nM with the addition of a FLAG tag to the light chain (Table 19). Therefore, all calculations of change in affinity relative to wild-type were performed using the median values of matching tagged wild-type constructs.

When measuring binding to CD-19, the sensorgrams of some antibodies could not be fit to a 1:1 binding model. In these cases, antibodies contained large amounts of ‘Fc Chain B’ half antibody that are prone to homodimerization, which caused avidity effects and obscured the Fab binding behavior. For the affected antibodies, binding affinities were further assessed in OAA format.

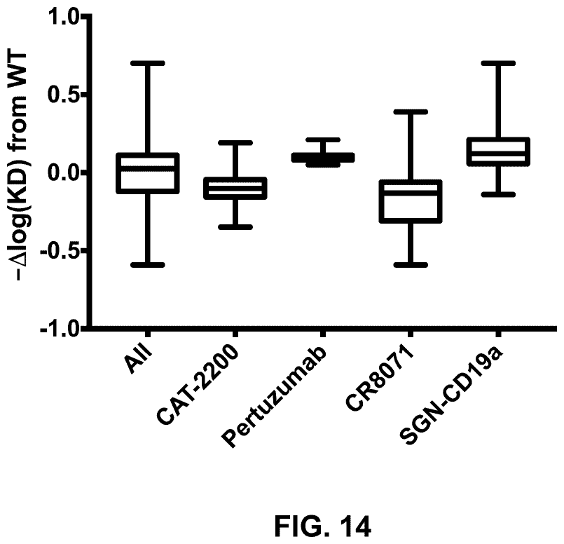

The majority of tested designs (36 designs corresponding to 96 bispecific antibodies made) exhibited KD values comparable to those of respective wild-type (Table 20 and 21, FIG. 14). Nine designed Fabs exhibited reduced affinity, between 2- and 3-fold compared to WT, and in a single designed Fab a 4-fold reduction in affinity was observed. Of these 10 Fabs, only one design (#34) was present twice, and 8 out of 10 affected Fabs were CR8071. The average difference in log(KD) from WT (−(log KD_antibody−log KD_wt)) for all warheads possessing designs was 0.003 with a standard deviation of 0.187 (e.g.-0.3 corresponds to 2-fold decrease in affinity, −0.6 corresponds to 4-fold decrease in affinity). These results are summarized in FIG. 14, which depicts a box plot of the change in affinity compared to parental antibody for the tested designs, for all designs tested (“All”), or by paratope (CAT-2200, pertuzumab, CR8071, or SGN-CD19a).

### Example 21: UltraPerformance Liquid Chromatography Size Exclusion Chromatography (UPLC-SEC) Profiles of Engineered Bispecific Antibodies Compared to Parental Antibodies

To assess the quality of the engineered bispecific antibodies, antibodies purified by preparative SEC as in Example 17 were subjected to UPLC-SEC. UPLC-SEC was performed using a Waters BEH200 SEC column (2.5 mL, 4.6×150 mm, stainless steel, 1.7 μm particles) set to 30° C. and mounted on a Waters Acquity UPLC system with a PDA detector. Run times consisted of 7 min and a total volume per injection of 2.8 mL with a running buffer of PBS and 0.02% Tween® 20 pH 7.4 at 0.4 ml/min. Elution was monitored by UV absorbance in the range 210-400 nm, and chromatograms were extracted at 280 nm. Peak integration was performed using Empower 3 software.

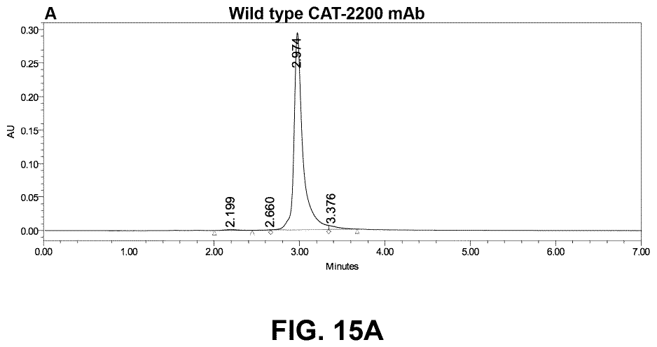

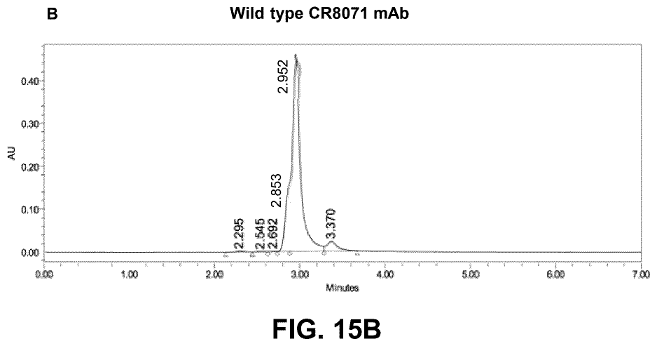

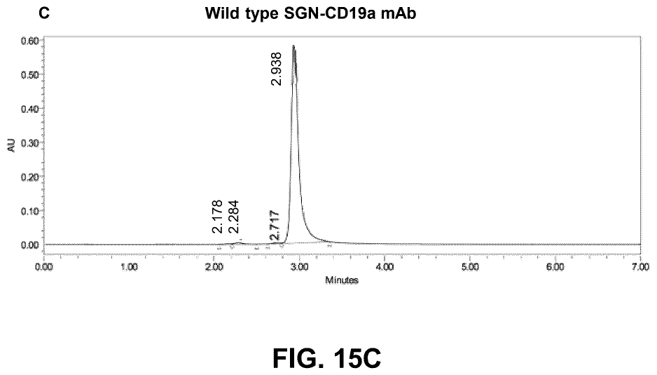

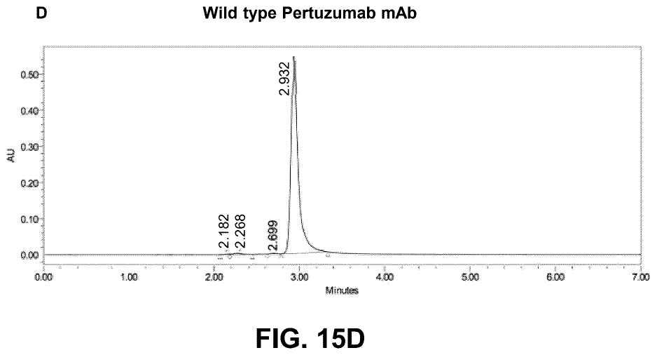

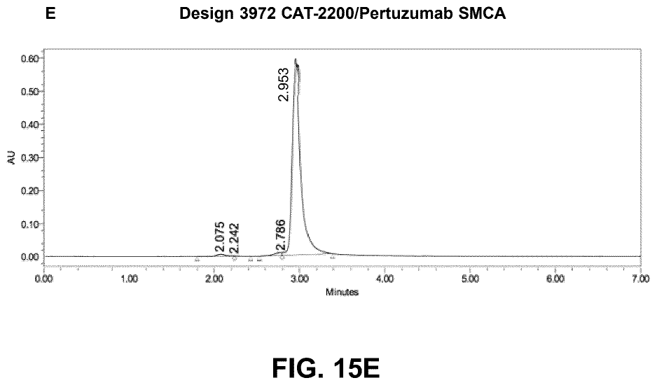

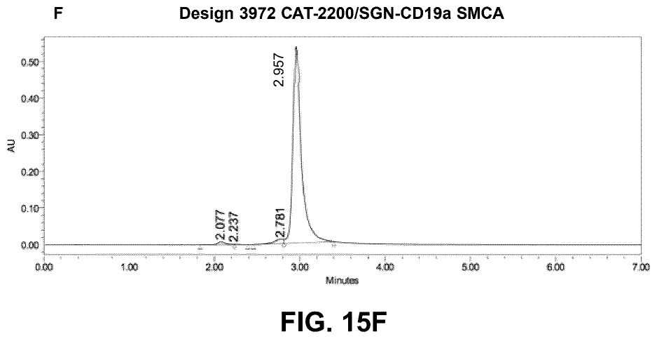

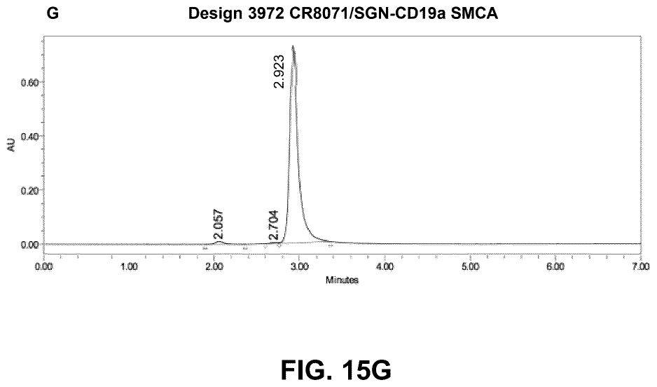

FIG. 15A-D depicts UPLC-SEC profiles of parental antibodies and FIG. 15E-G depicts those corresponding to design 3972 antibodies in three bispecific systems. Exemplary design 3972 was used here to demonstrate that the designed/engineered bispecific antibodies of high bispecific content (>80%) have UPLC-SEC profiles that are comparable to those of parental antibodies. For such engineered bispecific antibodies the median percentage of monomer species is 95.2% (i.e. little or no high molecular weight species were detected).

All references, issued patents, patent applications, and sequence accession numbers (e.g., Genbank® accession numbers) cited within the body of the instant specification are hereby incorporated by reference in their entirety, for all purposes.

TABLE 1
Criteria for Fab model

| Criteria | Importance |
|---|---|
| Human or humanized IgG1/κ or IgG1/λ | Similarity |
| Has commonly used V_{H} and V_{L} subgroups | |
| Framework close to germline | |
| 3D Structure represents a typical kappa or | |
| lambda conformation (low pairwise computed | |
| RMSD) | |
| Structure available for apo-and complexed Fab | Design |
| No major structural changes observed upon | |
| binding antigen | |
| Antigen binding can be readily assayed | Assay |

TABLE 2
Hotspot amino acid positions (marked by asterisk) at the
interface of the heavy and light chains in D3H44 (1JPT, a
typical Fab containing a kappa light chain) and CAT-2200
(2VXS, a typical Fab containing a lambda light chain).

| | | | |
|---|---|---|---|
| 1JPT | 1JPT Hotspot | 2VXS Hotspot | 2VXS |

Heavy Chain (Kabat numbering)

| | | | |
|---|---|---|---|
| F122 | * | * | F122 |
| S130 | * | | S130 |
| S134 | * | | S134 |
| A139 | * | | A139 |
| L143 | | * | L143 |
| K145 | * | * | K145 |
| H172 | * | * | H172 |
| F174 | * | * | F174 |
| P175 | * | | P175 |
| V177 | * | * | V177 |
| S188 | | * | S188 |
| K228 | * | * | K228 |

Light Chain (Kabat numbering)

| | | | |
|---|---|---|---|
| F116 | * | | T116 |
| F118 | * | * | F118 |
| P119 | * | | P119 |
| E123 | * | * | E123 |
| L135 | * | * | L135 |
| N160 | * | * | E160 |
| E161 | | | T161 |
| S162 | * | * | T162 |
| T164 | * | | P164 |
| T178 | | * | Y178 |

TABLE 3A
Kabat numbering of the heavy chain amino acid sequences
of D3H44, Pertuzumab, and CAT-2200 Fabs.
Heavy chain origin

| KABAT | | Pertuzumab | CAT-2200 |
|---|---|---|---|
| numbering | D3H44 | (Seq ID No. 1) | (Seq ID No. 3) |
| 1 | E | E | E |
| 2 | V | V | V |
| 3 | Q | Q | Q |
| 4 | L | L | L |
| 5 | V | V | L |
| 6 | E | E | E |
| 7 | S | S | S |
| 8 | G | G | G |
| 9 | G | G | G |
| 10 | G | G | G |
| 11 | L | L | L |
| 12 | V | V | V |
| 13 | Q | Q | Q |
| 14 | P | P | P |
| 15 | G | G | G |
| 16 | G | G | G |
| 17 | S | S | S |
| 18 | L | L | L |
| 19 | R | R | R |
| 20 | L | L | L |
| 21 | S | S | S |
| 22 | C | C | C |
| 23 | A | A | A |
| 24 | A | A | A |
| 25 | S | S | S |
| 26 | G | G | G |
| 27 | F | F | F |
| 28 | N | T | T |
| 29 | I | F | F |
| 30 | K | T | S |
| 31 | E | D | S |
| 32 | Y | Y | Y |
| 33 | Y | T | A |
| 34 | M | M | M |
| 35 | H | D | S |
| 36 | W | W | W |
| 37 | V | V | V |
| 38 | R | R | R |
| 39 | Q | Q | Q |
| 40 | A | A | A |
| 41 | P | P | P |
| 42 | G | G | G |
| 43 | K | K | K |
| 44 | G | G | G |
| 45 | L | L | L |
| 46 | E | E | E |
| 47 | W | W | W |
| 48 | V | V | V |
| 49 | G | A | S |
| 50 | L | D | A |
| 51 | I | V | I |
| 52 | D | N | S |
| 52A | P | P | G |
| 53 | E | N | S |
| 54 | Q | S | G |
| 55 | G | G | G |
| 56 | N | G | S |
| 57 | T | S | T |
| 58 | I | I | Y |
| 59 | Y | Y | Y |
| 60 | D | N | A |
| 61 | P | Q | D |
| 62 | K | R | S |
| 63 | F | F | V |
| 64 | Q | K | K |
| 65 | D | G | G |
| 66 | R | R | R |
| 67 | A | F | F |
| 68 | T | T | T |
| 69 | I | L | I |
| 70 | S | S | S |
| 71 | A | V | R |
| 72 | D | D | D |
| 73 | N | R | N |
| 74 | S | S | S |
| 75 | K | K | K |
| 76 | N | N | N |
| 77 | T | T | T |
| 78 | A | L | L |
| 79 | Y | Y | Y |
| 80 | L | L | L |
| 81 | Q | Q | Q |
| 82 | M | M | M |
| 82A | N | N | N |
| 82B | S | S | S |
| 82C | L | L | L |
| 83 | R | R | R |
| 84 | A | A | A |
| 85 | E | E | E |
| 86 | D | D | D |
| 87 | T | T | T |
| 88 | A | A | A |
| 89 | V | V | V |
| 90 | Y | Y | Y |
| 91 | Y | Y | Y |
| 92 | C | C | C |
| 93 | A | A | A |
| 94 | R | R | R |
| 95 | D | N | D |
| 96 | T | L | L |
| 97 | A | G | I |
| 98 | A | P | H |
| 99 | Y | S | G |
| 100 | F | F | V |
| 100A | — | Y | T |
| 100B | — | F | — |
| 101 | D | D | R |
| 102 | Y | Y | N |
| 103 | W | W | W |
| 104 | G | G | G |
| 105 | Q | Q | Q |
| 106 | G | G | G |
| 107 | T | T | T |
| 108 | L | L | L |
| 109 | V | V | V |
| 110 | T | T | T |
| 111 | V | V | V |
| 112 | S | S | S |
| 113 | S | S | S |
| 114 | A | A | A |
| 115 | S | S | S |
| 116 | T | T | T |
| 117 | K | K | K |
| 118 | G | G | G |
| 119 | P | P | P |
| 120 | S | S | S |
| 121 | V | V | V |
| 122 | F | F | F |
| 123 | P | P | P |
| 124 | L | L | L |
| 125 | A | A | A |
| 126 | P | P | P |
| 127 | S | S | S |
| 128 | S | S | S |
| 129 | K | K | K |
| 130 | S | S | S |
| 133 | T | T | T |
| 134 | S | S | S |
| 135 | G | G | G |
| 136 | G | G | G |
| 137 | T | T | T |
| 138 | A | A | A |
| 139 | A | A | A |
| 140 | L | L | L |
| 141 | G | G | G |
| 142 | C | C | C |
| 143 | L | L | L |
| 144 | V | V | V |
| 145 | K | K | K |
| 146 | D | D | D |
| 147 | Y | Y | Y |
| 148 | F | F | F |
| 149 | P | P | P |
| 150 | E | E | Q |
| 151 | P | P | P |
| 152 | V | V | V |
| 153 | T | T | T |
| 154 | V | V | V |
| 156 | S | S | S |
| 157 | W | W | W |
| 162 | N | N | N |
| 163 | S | S | S |
| 164 | G | G | G |
| 165 | A | A | A |
| 166 | L | L | L |
| 167 | T | T | T |
| 168 | S | S | S |
| 169 | G | G | G |
| 171 | V | V | V |
| 172 | H | H | H |
| 173 | T | T | T |
| 174 | F | F | F |
| 175 | P | P | P |
| 176 | A | A | A |
| 177 | V | V | V |
| 178 | L | L | L |
| 179 | Q | Q | Q |
| 180 | S | S | S |
| 182 | S | S | S |
| 183 | G | G | G |
| 184 | L | L | L |
| 185 | Y | Y | Y |
| 186 | S | S | S |
| 187 | L | L | L |
| 188 | S | S | S |
| 189 | S | S | S |
| 190 | V | V | V |
| 191 | V | V | V |
| 192 | T | T | T |
| 193 | V | V | V |
| 194 | P | P | P |
| 195 | S | S | S |
| 196 | S | S | S |
| 197 | S | S | S |
| 198 | L | L | L |
| 199 | G | G | G |
| 200 | T | T | T |
| 203 | Q | Q | Q |
| 205 | T | T | T |
| 206 | Y | Y | Y |
| 207 | I | I | I |
| 208 | C | C | C |
| 209 | N | N | N |
| 210 | V | V | V |
| 211 | N | N | N |
| 212 | H | H | H |
| 213 | K | K | K |
| 214 | P | P | P |
| 215 | S | S | S |
| 216 | N | N | N |
| 217 | T | T | T |
| 218 | K | K | K |
| 219 | V | V | V |
| 220 | D | D | D |
| 221 | K | K | K |
| 222 | K | K | K |
| 223 | V | V | V |
| 226 | E | E | E |
| 227 | P | P | P |
| 228 | K | K | K |
| 232 | S | S | S |
| 233 | C | C | C |
| 234 | D | D | D |
| 235 | K | K | K |
| 236 | T | T | T |
| 237 | H | H | H |
| 238 | T | T | T |

TABLE 3B
Light chain origin

| KABAT | | Pertuzumab | CAT-2200 |
|---|---|---|---|
| numbering | D3H44 | (Seq ID No. 2) | (Seq ID No. 4) |
| 1 | D | D | N |
| 2 | I | I | F |
| 3 | Q | Q | M |
| 4 | M | M | L |
| 5 | T | T | T |
| 6 | Q | Q | Q |
| 7 | S | S | P |
| 8 | P | P | H |
| 9 | S | S | S |
| 10 | S | S | — |
| 11 | L | L | V |
| 12 | S | S | S |
| 13 | A | A | E |
| 14 | S | S | S |
| 15 | V | V | P |
| 16 | G | G | G |
| 17 | D | D | K |
| 18 | R | R | T |
| 19 | V | V | V |
| 20 | T | T | T |
| 21 | I | I | I |
| 22 | T | T | S |
| 23 | C | C | C |
| 24 | R | K | T |
| 25 | A | A | R |
| 26 | S | S | S |
| 27 | R | Q | S |
| 27A | — | — | G |
| 27B | — | — | S |
| 28 | D | D | L |
| 29 | I | V | A |
| 30 | K | S | N |
| 31 | S | I | Y |
| 32 | Y | G | Y |
| 33 | L | V | V |
| 34 | N | A | Q |
| 35 | W | W | W |
| 36 | Y | Y | Y |
| 37 | Q | Q | Q |
| 38 | Q | Q | Q |
| 39 | K | K | R |
| 40 | P | P | P |
| 41 | G | G | G |
| 42 | K | K | S |
| 43 | A | A | S |
| 44 | P | P | P |
| 45 | K | K | T |
| 46 | V | L | I |
| 47 | L | L | V |
| 48 | I | I | I |
| 49 | Y | Y | F |
| 50 | Y | S | A |
| S1 | A | A | N |
| 52 | T | S | N |
| 53 | S | Y | Q |
| 54 | L | R | R |
| 55 | A | Y | P |
| 56 | E | T | S |
| 57 | G | G | G |
| 58 | V | V | V |
| 59 | P | P | P |
| 60 | S | S | D |
| 61 | R | R | R |
| 62 | F | F | F |
| 63 | S | S | S |
| 64 | G | G | G |
| 65 | S | S | S |
| 66 | G | G | I |
| 66A | — | — | D |
| 66B | — | — | S |
| 67 | S | S | S |
| 68 | G | G | S |
| 69 | T | T | N |
| 70 | D | D | S |
| 71 | Y | F | A |
| 72 | T | T | S |
| 73 | L | L | L |
| 74 | T | T | T |
| 75 | I | I | I |
| 76 | S | S | S |
| 77 | S | S | G |
| 78 | L | L | L |
| 79 | Q | Q | K |
| 80 | P | P | T |
| 81 | E | E | E |
| 82 | D | D | D |
| 83 | F | F | E |
| 84 | A | A | A |
| 85 | T | T | D |
| 86 | Y | Y | Y |
| 87 | Y | Y | Y |
| 88 | C | C | C |
| 89 | L | Q | Q |
| 90 | Q | Q | T |
| 91 | H | Y | Y |
| 92 | G | Y | D |
| 93 | E | I | P |
| 94 | S | Y | Y |
| 95 | P | P | S |
| 96 | W | Y | V |
| 97 | T | T | V |
| 98 | F | F | F |
| 99 | G | G | G |
| 100 | Q | Q | G |
| 101 | G | G | G |
| 102 | T | T | T |
| 103 | K | K | K |
| 104 | V | V | L |
| 105 | E | E | T |
| 106 | I | I | V |
| 106A | — | — | L |
| 107 | K | K | G |
| 108 | R | R | Q |
| 109 | T | T | P |
| 110 | V | V | K |
| 111 | A | A | A |
| 112 | A | A | A |
| 113 | P | P | P |
| 114 | S | S | S |
| 115 | V | V | V |
| 116 | F | F | T |
| 117 | I | I | L |
| 118 | F | F | F |
| 119 | P | P | P |
| 120 | P | P | P |
| 121 | S | S | S |
| 122 | D | D | S |
| 123 | E | E | E |
| 124 | Q | Q | E |
| 125 | L | L | L |
| 126 | K | K | Q |
| 127 | S | S | A |
| 128 | G | G | N |
| 129 | T | T | K |
| 130 | A | A | A |
| 131 | S | S | T |
| 132 | V | V | L |
| 133 | V | V | V |
| 134 | C | C | C |
| 135 | L | L | L |
| 136 | L | L | I |
| 137 | N | N | S |
| 138 | N | N | D |
| 139 | F | F | F |
| 140 | Y | Y | Y |
| 141 | P | P | P |
| 142 | R | R | G |
| 143 | E | E | A |
| 144 | A | A | V |
| 145 | K | K | T |
| 146 | V | V | V |
| 147 | Q | Q | A |
| 148 | W | W | W |
| 149 | K | K | K |
| 150 | V | V | A |
| 1S1 | D | D | D |
| 152 | N | N | S |
| 153 | A | A | S |
| 154 | L | L | P |
| 155 | Q | Q | V |
| 156 | S | S | K |
| 157 | G | G | A |
| 158 | N | N | G |
| 159 | S | S | V |
| 160 | Q | Q | E |
| 161 | E | E | T |
| 162 | S | S | T |
| 163 | V | V | T |
| 164 | T | T | P |
| 165 | E | E | S |
| 166 | Q | Q | K |
| 167 | D | D | Q |
| 168 | S | S | S |
| 169 | K | K | — |
| 170 | D | D | N |
| 171 | S | S | N |
| 172 | T | T | K |
| 173 | Y | Y | Y |
| 174 | S | S | A |
| 175 | L | L | A |
| 176 | S | S | S |
| 177 | S | S | S |
| 178 | T | T | Y |
| 179 | L | L | L |
| 180 | T | T | S |
| 181 | L | L | L |
| 182 | S | S | T |
| 183 | K | K | P |
| 184 | A | A | E |
| 185 | D | D | Q |
| 186 | Y | Y | W |
| 187 | E | E | K |
| 188 | K | K | S |
| 189 | H | H | H |
| 190 | K | K | R |
| 191 | V | V | S |
| 192 | Y | Y | Y |
| 193 | A | A | S |
| 194 | C | C | C |
| 195 | E | E | Q |
| 196 | V | V | V |
| 197 | T | T | T |
| 198 | H | H | H |
| 199 | Q | Q | E |
| 200 | G | G | G |
| 201 | L | L | — |
| 202 | S | S | — |
| 203 | S | S | S |
| 204 | P | P | T |
| 205 | V | V | V |
| 206 | T | T | E |
| 207 | K | K | K |
| 208 | S | S | T |
| 209 | F | F | V |
| 210 | N | N | A |
| 211 | R | R | P |
| 212 | G | G | T |
| 213 | E | E | E |
| 214 | C | C | C |
| 215 | — | — | S |

TABLE 3C
Amino acid and DNA sequences

| | DESCRIPTION (amino | |
|---|---|---|
| SEQ | acid residues | |
| ID | numbered with | |
| NO: | respect to sequence) | SEQUENCE |
| 1 | Pertuzumab heavy | EVQLVESGGGLVQPGGSLRLSCAASGFTFTDYTMDWVRQAPGKGLEWVADVNPNSGGSIYNQRF |
| | chain Fab (Domain | KGRFTLSVDRSKNTLYLQMNSLRAEDTAVYYCARNLGPSFYFDYWGQGTLVTVSSASTKGPSVFPLA |
| | boundaries: VH; E1- | PSSKSTSGGTAALGCLVKDYFPEPVTVSWNSGALTSGVHTFPAVLQSSGLYSLSSVVTVPSSSLGTQT |
| | S119, CH1; A120- | YICNVNHKPSNTKVDKKVEPKSCDKTHT |
| | V217, Hinge (partial); | |
| | E218-T227) | |
| 2 | Pertuzumab light | DIQMTQSPSSLSASVGDRVTITCKASQDVSIGVAWYQQKPGKAPKLLIYSASYRYTGVPSRFSGSGS |
| | chain (kappa) | GTDFTLTISSLQPEDFATYYCQQYYIYPYTFGQGTKVEIKRTVAAPSVFIFPPSDEQLKSGTASVVCLLN |
| | (Domain boundaries: | NFYPREAKVQWKVDNALQSGNSQESVTEQDSKDSTYSLSSTLTLSKADYEKHKVYACEVTHQGLSSP |
| | VL; D1-K107, CL; | VTKSFNRGEC |
| | R108-C214) | |
| 3 | CAT-2200 heavy chain | EVQLLESGGGLVQPGGSLRLSCAASGFTFSSYAMSWVRQAPGKGLEWVSAISGSGGSTYYADSVKG |
| | Fab (Domain | RFTISRDNSKNTLYLQMNSLRAEDTAVYYCARDLIHGVTRNWGQGTLVTVSSASTKGPSVFPLAPSS |
| | boundaries: VH; E1- | KSTSGGTAALGCLVKDYFPQPVTVSWNSGALTSGVHTFPAVLQSSGLYSLSSVVTVPSSSLGTQTYIC |
| | S118, CH1; A119- | NVNHKPSNTKVDKKVEPKSCDKTHT |
| | V216, Hinge(partial); | |
| | E217-T226) | |
| 4 | CAT-2200 light chain | NFMLTQPHSVSESPGKTVTISCTRSSGSLANYYVQWYQQRPGSSPTIVIFANNQRPSGVPDRFSGSI |
| | (lambda) (Domain | DSSSNSASLTISGLKTEDEADYYCQTYDPYSVVFGGGTKLTVLGQPKAAPSVTLFPPSSEELQANKATL |
| | boundaries: VL; N1- | VCLISDFYPGAVTVAWKADSSPVKAGVETTTPSKQSNNKYAASSYLSLTPEQWKSHRSYSCQVTHE |
| | L110, CL; G111-S216) | GSTVEKTVAPTECS |
| 5 | IgG1 Fc (Domain | CPPCPAPELLGGPSVFLFPPKPKDTLMISRTPEVTCVVVDVSHEDPEVKFNWYVDGVEVHNAKTKPR |
| | boundaries: Hinge | EEQYNSTYRVVSVLTVLHQDWLNGKEYKCKVSNKALPAPIEKTISKAKGQPREPQVYTLPPSRDELTK |
| | (partial); C1-P5, CH2; | NQVSLTCLVKGFYPSDIAVEWESNGQPENNYKTTPPVLDSDGSFFLYSKLTVDKSRWQQGNVFSCS |
| | A6-K115, CH3; G116- | VMHEALHNHYTQKSLSLSPGK |
| | K222) | |
| 6 | IgG1 Fc | TGCCCTCCCTGTCCAGCTCCAGAACTGCTGGGAGGACCTAGCGTGTTCCTGTTTCCCCCTAAGCCA |
| | | AAAGACACTCTGATGATTTCCAGGACTCCCGAGGTGACCTGCGTGGTGGTGGACGTGTCTCACG |
| | | AGGACCCCGAAGTGAAGTTCAACTGGTACGTGGATGGCGTGGAAGTGCATAATGCTAAGACAA |
| | | AACCAAGAGAGGAACAGTACAACTCCACTTATCGCGTCGTGAGCGTGCTGACCGTGCTGCACCA |
| | | GGACTGGCTGAACGGGAAGGAGTATAAGTGCAAAGTCAGTAATAAGGCCCTGCCTGCTCCAATC |
| | | GAAAAAACCATCTCTAAGGCCAAAGGCCAGCCAAGGGAGCCCCAGGTGTACACACTGCCACCCA |
| | | GCAGAGACGAACTGACCAAGAACCAGGTGTCCCTGACATGTCTGGTGAAAGGCTTCTATCCTAG |
| | | TGATATTGCTGTGGAGTGGGAATCAAATGGACAGCCAGAGAACAATTACAAGACCACACCTCCA |
| | | GTGCTGGACAGCGATGGCAGCTTCTTCCTGTATTCCAAGCTGACAGTGGATAAATCTCGATGGCA |
| | | GCAGGGGAACGTGTTTAGTTGTTCAGTGATGCATGAAGCCCTGCACAATCATTACACTCAGAAG |
| | | AGCCTGTCCCTGTCTCCCGGCAAA |
| 7 | Pertuzumab heavy | GAAGTGCAGCTGGTCGAATCTGGAGGAGGACTGGTGCAGCCAGGAGGGTCCCTGCGCCTGTCTT |
| | chain Fab | GCGCCGCTAGTGGCTTCACTTTTACCGACTACACCATGGATTGGGTGCGACAGGCACCTGGAAA |
| | | GGGCCTGGAGTGGGTCGCCGATGTGAACCCAAATAGCGGAGGCTCCATCTACAACCAGCGGTTC |
| | | AAGGGCCGGTTCACCCTGTCAGTGGACCGGAGCAAAAACACCCTGTATCTGCAGATGAATAGCC |
| | | TGCGAGCCGAAGATACTGCTGTGTACTATTGCGCCCGGAATCTGGGGCCCTCCTTCTACTTTGACT |
| | | ATTGGGGGCAGGGAACTCTGGTCACCGTGAGCTCCGCCTCCACCAAGGGACCTTCTGTGTTCCCA |
| | | CTGGCTCCCTCTAGTAAATCCACATCTGGGGGAACTGCAGCCCTGGGCTGTCTGGTGAAGGACTA |
| | | CTTCCCAGAGCCCGTCACAGTGTCTTGGAACAGTGGCGCTCTGACTTCTGGGGTCCACACCTTTCC |
| | | TGCAGTGCTGCAGTCAAGCGGGCTGTACAGCCTGTCCTCTGTGGTCACCGTGCCAAGTTCAAGCC |
| | | TGGGAACACAGACTTATATCTGCAACGTGAATCACAAGCCATCCAATACAAAAGTCGACAAGAA |
| | | AGTGGAACCCAAGTCTTGTGATAAAACCCATACA |
| 8 | Pertuzumab light | GATATTCAGATGACCCAGTCCCCAAGCTCCCTGAGTGCCTCAGTGGGCGACCGAGTCACCATCAC |
| | chain (kappa) | ATGCAAGGCTTCCCAGGATGTGTCTATTGGAGTCGCATGGTACCAGCAGAAGCCAGGCAAAGCA |
| | | CCCAAGCTGCTGATCTATAGCGCCTCCTACCGGTATACCGGCGTGCCCTCTAGATTCTCTGGCAGT |
| | | GGGTCAGGAACAGACTTTACTCTGACCATCTCTAGTCTGCAGCCTGAGGATTTCGCTACCTACTAT |
| | | TGCCAGCAGTACTATATCTACCCATATACCTTTGGCCAGGGGACAAAAGTGGAGATCAAGAGGA |
| | | CTGTGGCCGCTCCCTCCGTCTTCATTTTTCCCCCTTCTGACGAACAGCTGAAAAGTGGCACAGCCA |
| | | GCGTGGTCTGTCTGCTGAACAATTTCTACCCTCGCGAAGCCAAAGTGCAGTGGAAGGTCGATAA |
| | | CGCTCTGCAGAGCGGCAACAGCCAGGAGTCTGTGACTGAACAGGACAGTAAAGATTCAACCTAT |
| | | AGCCTGTCAAGCACACTGACTCTGAGCAAGGCAGACTACGAGAAGCACAAAGTGTATGCCTGCG |
| | | AAGTCACACATCAGGGGCTGTCCTCTCCTGTGACTAAGAGCTTTAACAGAGGAGAGTGT |
| 9 | CAT-2200 heavy chain | GAGGTGCAGCTGCTGGAATCTGGGGGGGGCCTGGTGCAGCCTGGGGGGTCCCTGAGACTGTCA |
| | Fab | TGTGCTGCCAGCGGGTTTACTTTCAGCTCCTACGCTATGTCCTGGGTGCGACAGGCACCCGGGAA |
| | | GGGACTGGAGTGGGTCTCTGCAATCAGTGGGTCAGGCGGGAGTACTTACTATGCCGACAGCGT |
| | | GAAGGGACGGTTCACTATCTCAAGAGATAACAGCAAGAACACCCTGTATCTGCAGATGAACAGC |
| | | CTGAGAGCAGAAGACACAGCCGTGTACTATTGCGCCAGGGATCTGATCCACGGAGTCACTCGCA |
| | | ATTGGGGCCAGGGGACTCTGGTGACCGTCTCTAGTGCTAGCACAAAGGGGCCCTCTGTGTTTCCA |
| | | CTGGCCCCCTCAAGCAAAAGCACATCCGGAGGAACTGCAGCTCTGGGATGTCTGGTGAAGGACT |
| | | ACTTCCCCCAGCCTGTGACCGTCTCTTGGAACAGTGGAGCCCTGACCAGCGGCGTGCACACATTT |
| | | CCTGCTGTCCTGCAGTCCTCTGGCCTGTACTCCCTGAGTTCAGTGGTCACAGTGCCTAGCTCCTCTC |
| | | TGGGGACCCAGACATATATTTGCAACGTGAATCATAAACCAAGCAACACTAAGGTCGACAAGAA |
| | | AGTGGAGCCCAAGAGCTGTGATAAAACTCATACC |
| 10 | CAT-2200 light chain | AACTTTATGCTGACTCAGCCCCACTCCGTGTCCGAGAGCCCTGGCAAAACTGTGACTATTTCATGT |
| | (lambda) | ACCCGATCATCTGGAAGCCTGGCCAACTACTATGTGCAGTGGTACCAGCAGAGGCCAGGCAGCT |
| | | CCCCCACTATCGTGATTTTCGCTAACAATCAGCGGCCTTCCGGCGTCCCAGACAGATTTTCCGGGT |
| | | CTATCGATTCTAGTTCAAATAGTGCATCACTGACTATTTCCGGGCTGAAGACCGAGGACGAAGCC |
| | | GATTACTATTGCCAGACCTACGACCCCTATTCTGTGGTCTTCGGCGGGGGAACCAAGCTGACAGT |
| | | GCTGGGACAGCCAAAAGCGGCGCCCAGTGTCACACTGTTTCCCCCTAGCTCCGAGGAACTGCAG |
| | | GCTAACAAAGCAACACTGGTGTGTCTGATCAGCGACTTCTACCCTGGAGCTGTGACTGTCGCCTG |
| | | GAAGGCTGATTCTAGTCCAGTGAAAGCAGGCGTCGAGACCACAACTCCCTCTAAGCAGAGTAAC |
| | | AACAAGTACGCAGCCTCAAGCTATCTGTCACTGACCCCAGAACAGTGGAAGAGCCACCGGAGCT |
| | | ATTCCTGCCAGGTCACTCACGAAGGCTCCACTGTCGAGAAAACCGTCGCTCCCACCGAATGTTCA |
| 11 | Human IgG1 Fc | APELLGGPSVFLFPPKPKDTLMISRTPEVTCVVVDVSHEDPEVKFNWYVDGVEVHNAKTKPREEQY |
| | sequence 231-447 | NSTYRVVSVLTVLHQDWLNGKEYKCKVSNKALPAPIEKTISKAKGQPREPQVYTLPPSRDELTKNQV |
| | (EU-numbering), | SLTCLVKGFYPSDIAVEWESNGQPENNYKTTPPVLDSDGSFFLYSKLTVDKSRWQQGNVFSCSVMH |
| | without hinge | EALHNHYTQKSLSLSPGK |
| 12 | SGN-CD19a heavy | QVTLRESGPALVKPTQTLTLTCTFSGFSLSTSGMGVGWIRQPPGKALEWLAHIWWDDDKRYNPALK |
| | chain Fab (Domain | SRLTISKDTSKNQVVLTMTNMDPVDTAAYYCARMELWSYYFDYWGQGTLVTVSSASTKGPSVFPL |
| | boundaries: VH; Q1- | APSSKSTSGGTAALGCLVKDYFPEPVTVSWNSGALTSGVHTFPAVLQSSGLYSLSSVVTVPSSSLGTQ |
| | S120, CH1; A121- | TYICNVNHKPSNTKVDKKVEPKSCDKTHT |
| | V218, Hinge (partial); | |
| | E219-T228) | |
| 13 | SGN-CD19a light chain | EIVLTQSPATLSLSPGERATLSCSASSSVSYMHWYQQKPGQAPRLLIYDTSKLASGIPARFSGSGSGTD |
| | (kappa) (Domain | FTLTISSLEPEDFAVYYCFQGSVYPFTFGQGTKLEIKRTVAAPSVFIFPPSDEQLKSGTASVVCLLNNFYP |
| | boundaries: VL; E1- | REAKVQWKVDNALQSGNSQESVTEQDSKDSTYSLSSTLTLSKADYEKHKVYACEVTHQGLSSPVTKS |
| | K106, CL; R107- | FNRGEC |
| | C213) | |
| 14 | CR8071 heavy chain | QVQLVQSGAEVKKPGASVRVSCRASGYIFTESGITWVRQAPGQGLEWMGWISGYSGDTKYAQKL |
| | Fab (Domain | QGRVTMTKDTSTTTAYMELRSLRYDDTAVYYCARDVQYSGSYLGAYYFDYWSPGTLVTVSSASTKG |
| | boundaries: VH; Q1- | PSVFPLAPSSKSTSGGTAALGCLVKDYFPEPVTVSWNSGALTSGVHTFPAVLQSSGLYSLSSVVTVPSS |
| | S125, CH1; A126- | SLGTQTYICNVNHKPSNTKVDKKVEPKSCDKTHT |
| | V223, Hinge (partial); | |
| | E224-T233) | |
| 15 | CR8071 light chain | QSVLTQPPSASGTPGQRVTISCSGSSSNIGTNYVYWYQQFPGTAPKLLIYRSYQRPSGVPDRFSGSKS |
| | (lambda) (Domain | GSSASLAISGLQSEDEADYYCATWDDSLDGWVFGGGTKLTVLRQPKAAPSVTLFPPSSEELQANKAT |
| | boundaries: VL; Q1- | LVCLISDFYPGAVTVAWKADSSPVKAGVETTTPSKQSNNKYAASSYLSLTPEQWKSHRSYSCQVTHE |
| | L110, CL; R111-S216) | GSTVEKTVAPTECS |
| 16 | SGN-CD19a heavy | CAGGTGACACTGAGAGAATCCGGCCCAGCCCTGGTGAAGCCCACTCAGACCCTGACACTGACTT |
| | chain Fab | GCACCTTCTCTGGGTTTTCCCTGTCTACAAGTGGGATGGGAGTGGGATGGATCAGGCAGCCACCT |
| | | GGAAAAGCCCTGGAGTGGCTGGCTCACATTTGGTGGGACGATGACAAGCGGTACAACCCAGCA |
| | | CTGAAAAGCAGACTGACAATCAGCAAGGATACTTCCAAAAACCAGGTGGTCCTGACAATGACTA |
| | | ATATGGACCCCGTGGACACAGCCGCTTACTATTGCGCCCGCATGGAACTGTGGAGCTACTATTTC |
| | | GACTACTGGGGGCAGGGAACACTGGTCACTGTGAGCTCCGCTAGCACTAAGGGGCCTTCCGTGT |
| | | TTCCACTGGCTCCCTCTAGTAAATCCACCTCTGGAGGCACAGCTGCACTGGGATGTCTGGTGAAG |
| | | GATTACTTCCCTGAACCAGTCACAGTGAGTTGGAACTCAGGGGCTCTGACAAGTGGAGTCCATA |
| | | CTTTTCCCGCAGTGCTGCAGTCAAGCGGACTGTACTCCCTGTCCTCTGTGGTCACCGTGCCTAGTT |
| | | CAAGCCTGGGCACCCAGACATATATCTGCAACGTGAATCACAAGCCATCAAATACAAAAGTCGA |
| | | CAAGAAAGTGGAGCCCAAGAGCTGTGATAAAACTCATACC |
| 17 | SGN-CD19a light chain | GAGATCGTGCTGACCCAGTCTCCAGCCACACTGTCTCTGAGCCCAGGAGAGAGGGCCACCCTGTC |
| | (kappa) | CTGCTCTGCCAGCTCCAGCGTGAGCTACATGCACTGGTATCAGCAGAAGCCAGGACAGGCCCCT |
| | | AGGCTGCTGATCTACGACACCAGCAAGCTGGCCTCCGGCATCCCCGCAAGATTCAGCGGCTCCG |
| | | GCTCTGGCACAGACTTTACCCTGACAATCAGCTCCCTGGAGCCTGAGGATTTCGCCGTGTACTATT |
| | | GTTTTCAGGGCAGCGTGTATCCATTCACCTTTGGCCAGGGCACAAAGCTGGAGATCAAGCGGAC |
| | | AGTGGCGGCGCCCAGTGTCTTCATTTTTCCCCCTAGCGACGAACAGCTGAAGTCTGGGACAGCCA |
| | | GTGTGGTCTGTCTGCTGAACAACTTCTACCCTAGAGAGGCTAAAGTGCAGTGGAAGGTCGATAA |
| | | CGCACTGCAGTCCGGAAATTCTCAGGAGAGTGTGACTGAACAGGACTCAAAAGATAGCACCTAT |
| | | TCCCTGTCAAGCACACTGACTCTGAGCAAGGCCGACTACGAGAAGCATAAAGTGTATGCTTGTG |
| | | AAGTCACCCACCAGGGGCTGAGTTCACCAGTCACAAAATCATTCAACAGAGGGGAGTGC |
| 18 | CR8071 heavy chain | CAGGTGCAGCTGGTCCAGTCCGGGGCTGAAGTGAAAAAACCTGGGGCATCCGTGCGGGTGTCA |
| | Fab | TGTCGGGCAAGCGGGTATATCTTTACTGAGTCTGGAATCACCTGGGTGAGGCAGGCTCCCGGAC |
| | | AGGGACTGGAATGGATGGGATGGATTTCTGGATACAGTGGCGACACAAAGTATGCACAGAAAC |
| | | TGCAGGGCCGCGTCACCATGACAAAGGATACTTCAACCACAACTGCCTACATGGAGCTGCGGAG |
| | | CCTGAGATATGACGATACAGCCGTGTACTATTGCGCCCGGGACGTGCAGTACAGCGGGTCCTAC |
| | | CTGGGGGCATACTACTTCGATTACTGGTCACCTGGAACTCTGGTCACCGTCTCTTCAGCTAGCACC |
| | | AAGGGCCCTTCTGTGTTTCCACTGGCACCCTCAAGCAAAAGCACCTCCGGAGGAACAGCAGCACT |
| | | GGGATGTCTGGTCAAGGACTATTTCCCCGAGCCTGTGACCGTCTCATGGAATAGCGGCGCACTG |
| | | ACTAGTGGGGTGCACACCTTTCCCGCCGTCCTGCAGTCCTCTGGGCTGTACAGCCTGAGTTCAGT |
| | | GGTCACAGTGCCAAGCTCCTCTCTGGGAACTCAGACCTATATCTGCAACGTCAATCATAAACCCA |
| | | GCAACACAAAGGTCGACAAGAAAGTGGAGCCCAAGAGCTGTGATAAAACTCATACC |
| 19 | CR8071 light chain | CAGAGCGTCCTGACTCAGCCTCCCTCCGCCTCCGGAACACCTGGGCAGAGAGTGACTATCTCCTG |
| | (lambda) | TAGCGGATCAAGCTCAAACATTGGAACCAACTACGTGTATTGGTACCAGCAGTTCCCCGGCACA |
| | | GCTCCTAAGCTGCTGATCTATCGGAGCTACCAGAGACCAAGCGGGGTCCCCGACAGGTTTTCTGG |
| | | CAGTAAATCAGGGAGCTCCGCCAGCCTGGCTATTTCCGGCCTGCAGTCTGAGGACGAAGCAGAT |
| | | TACTATTGCGCCACCTGGGACGATTCCCTGGATGGATGGGTCTTCGGCGGCGGCACAAAACTGA |
| | | CCGTCCTGAGGCAGCCAAAGGCGGCGCCCAGTGTCACACTGTTTCCCCCTAGCTCCGAGGAACTG |
| | | CAGGCTAACAAAGCAACACTGGTGTGTCTGATCAGCGACTTCTACCCTGGAGCTGTGACTGTCGC |
| | | CTGGAAGGCTGATTCTAGTCCAGTGAAAGCAGGCGTCGAGACCACAACTCCCTCTAAGCAGAGT |
| | | AACAACAAGTACGCAGCCTCAAGCTATCTGTCACTGACCCCAGAACAGTGGAAGAGCCACCGGA |
| | | GCTATTCCTGCCAGGTCACTCACGAAGGCTCCACTGTCGAGAAAACCGT |
| | | CGCTCCCACCGAATGTTCA |

TABLE 4A
K-L design libary

| Unique | H1 mutation | L1 mutation | H2 mutation | L2 mutation | | |
|---|---|---|---|---|---|---|
| identifier | (Kabat) | (Kabat) | (Kabat) | (Kabat) | H1L1 Fab | H2L2 Fab |
| 10771-11335 | — | — | S186I_S188W | S176V | CAT-2200 | Pertuzumab |
| 10771-11360 | — | — | S186L_S188W | S176V | CAT-2200 | Pertuzumab |
| 10776-11312 | D146T_V177D_S188D | S176K_Y178R | L143K | V133D | CAT-2200 | Pertuzumab |
| 10776-11321 | D146T_V177D_S188D | S176K_Y178R | L143S_S186K_S188T | V133D | CAT-2200 | Pertuzumab |
| 10776-11353 | D146T_V177D_S188D | S176K_Y178R | S186K_S188T | V133D | CAT-2200 | Pertuzumab |
| 10780-11417 | F174G | S176F | V190F | — | CAT-2200 | Pertuzumab |
| 10781-11300 | F174G | S176F | L143I_V190F | L135A | CAT-2200 | Pertuzumab |
| 10781-11419 | F174G | S176F | V190F | L135A | CAT-2200 | Pertuzumab |
| 10786-11299 | F174G | T116F_S176F | L143I_V190F | — | CAT-2200 | Pertuzumab |
| 10786-11418 | F174G | T116F_S176F | V190F | — | CAT-2200 | Pertuzumab |
| 10787-11301 | F174G | T116F_S176F | L143I_V190F | L135A | CAT-2200 | Pertuzumab |
| 10787-11420 | F174G | T116F_S176F | V190F | L135A | CAT-2200 | Pertuzumab |
| 10788-11422 | F174G | T116F_S176F | V190F | L135A_T178F | CAT-2200 | Pertuzumab |
| 10792-11305 | F174G | T116F_S176F | L143I_V190F | T178F | CAT-2200 | Pertuzumab |
| 10792-11424 | F174G | T116F_S176F | V190F | T178F | CAT-2200 | Pertuzumab |
| 10793-11375 | F174G | T116F_S176F | S188F | V133A | CAT-2200 | Pertuzumab |
| 10795-11390 | K145T_S188E | Y178K | S188K | S176D_T178E | CAT-2200 | Pertuzumab |
| 10796-11396 | K145T_S188E | Y178K | S188K | S176E_T178E | CAT-2200 | Pertuzumab |
| 10798-11256 | K145T_S188E | Y178K | L124R_Q179K | V133G_S176D_T180E | CAT-2200 | Pertuzumab |
| 10798-11268 | K145T_S188E | Y178K | L124R_S186R | V133G_S176D_T180E | CAT-2200 | Pertuzumab |
| 10800-11416 | K145T_S188E | Y178K | V177I_S188K | V133L_S176D_T178E | CAT-2200 | Pertuzumab |
| 10806-11326 | K145T_V177D_S188D | S176K_Y178K | Q179K | Q124E_Q160E_T180E | CAT-2200 | Pertuzumab |
| 10806-11369 | K145T_V177D_S188D | S176K_Y178K | S186R | Q124E_Q160E_T180E | CAT-2200 | Pertuzumab |
| 10808-11308 | K145T_V177D_S188D | S176K_Y178K | L143K | Q124E_V133D | CAT-2200 | Pertuzumab |
| 10808-11315 | K145T_V177D_S188D | S176K_Y178K | L143R | Q124E_V133D | CAT-2200 | Pertuzumab |
| 10809-11378 | K145T_V177D_S188D | S176K_Y178K | S188K | S131D_V133G_T178F | CAT-2200 | Pertuzumab |
| 10810-11383 | K145T_V177D_S188D | S176K_Y178K | S188K | S131E_V133G_T178F | CAT-2200 | Pertuzumab |
| 10811-11386 | K145T_V177D_S188D | S176K_Y178K | S188K | S176D_T178E | CAT-2200 | Pertuzumab |
| 10812-11393 | K145T_V177D_S188D | S176K_Y178K | S188K | S176E_T178E | CAT-2200 | Pertuzumab |
| 10813-11311 | K145T_V177D_S188D | S176K_Y178K | L143K | V133D | CAT-2200 | Pertuzumab |
| 10813-11320 | K145T_V177D_S188D | S176K_Y178K | L143S_S186K_S188T | V133D | CAT-2200 | Pertuzumab |
| 10813-11352 | K145T_V177D_S188D | S176K_Y178K | S186K_S188T | V133D | CAT-2200 | Pertuzumab |
| 10814-11233 | K145T_V177D_S188D | S176K_Y178K | L124R | V133G_S176D | CAT-2200 | Pertuzumab |
| 10816-11253 | K145T_V177D_S188D | S176K_Y178K | L124R_Q179K | V133G_S176D_T180E | CAT-2200 | Pertuzumab |
| 10816-11265 | K145T_V177D_S188D | S176K_Y178K | L124R_S186R | V133G_S176D_T180E | CAT-2200 | Pertuzumab |
| 10818-11404 | K145T_V177D_S188D | S176K_Y178K | V177I_S188K | V133I_S176D_T178E | CAT-2200 | Pertuzumab |
| 10819-11407 | K145T_V177D_S188D | S176K_Y178K | V177I_S188K | V133I_S176E_T178E | CAT-2200 | Pertuzumab |
| 10820-11413 | K145T_V177D_S188D | S176K_Y178K | V177I_S188K | V133L_S176D_T178E | CAT-2200 | Pertuzumab |
| 10827-11373 | K145T_V177D_S188D | S176K_Y178L | S186R_S188W | Q124E_Q160E_S176A_ | CAT-2200 | Pertuzumab |
| | | | | T178A_T180E | | |
| 10828-11327 | K145T_V177D_S188D | S176K_Y178L | Q179K | Q124E_Q160E_T180E | CAT-2200 | Pertuzumab |
| 10828-11370 | K145T_V177D_S188D | S176K_Y178L | S186R | Q124E_Q160E_T180E | CAT-2200 | Pertuzumab |
| 10830-11309 | K145T_V177D_S188D | S176K_Y178L | L143K | Q124E_V133D | CAT-2200 | Pertuzumab |
| 10830-11316 | K145T_V177D_S188D | S176K_Y178L | L143R | Q124E_V133D | CAT-2200 | Pertuzumab |
| 10831-11379 | K145T_V177D_S188D | S176K_Y178L | S188K | S131D_V133G_T178F | CAT-2200 | Pertuzumab |
| 10832-11384 | K145T_V177D_S188D | S176K_Y178L | S188K | S131E_V133G_T178F | CAT-2200 | Pertuzumab |
| 10833-11387 | K145T_V177D_S188D | S176K_Y178L | S188K | S176D_T178E | CAT-2200 | Pertuzumab |
| 10834-11394 | K145T_V177D_S188D | S176K_Y178L | S188K | S176E_T178E | CAT-2200 | Pertuzumab |
| 10835-11234 | K145T_V177D_S188D | S176K_Y178L | L124R | V133G_S176D | CAT-2200 | Pertuzumab |
| 10837-11254 | K145T_V177D_S188D | S176K_Y178L | L124R_Q179K | V133G_S176D_T180E | CAT-2200 | Pertuzumab |
| 10837-11266 | K145T_V177D_S188D | S176K_Y178L | L124R_S186R | V133G_S176D_T180E | CAT-2200 | Pertuzumab |
| 10840-11408 | K145T_V177D_S188D | S176K_Y178L | V177I_S188K | V133I_S176E_T178E | CAT-2200 | Pertuzumab |
| 10848-11328 | K145T_V177D_S188D | S176K_Y178R | Q179K | Q124E_Q160E_T180E | CAT-2200 | Pertuzumab |
| 10848-11371 | K145T_V177D_S188D | S176K_Y178R | S186R | Q124E_Q160E_T180E | CAT-2200 | Pertuzumab |
| 10850-11310 | K145T_V177D_S188D | S176K_Y178R | L143K | Q124E_V133D | CAT-2200 | Pertuzumab |
| 10850-11317 | K145T_V177D_S188D | S176K_Y178R | L143R | Q124E_V133D | CAT-2200 | Pertuzumab |
| 10851-11380 | K145T_V177D_S188D | S176K_Y178R | S188K | S131D_V133G_T178F | CAT-2200 | Pertuzumab |
| 10852-11385 | K145T_V177D_S188D | S176K_Y178R | S188K | S131E_V133G_T178F | CAT-2200 | Pertuzumab |
| 10853-11388 | K145T_V177D_S188D | S176K_Y178R | S188K | S176D_T178E | CAT-2200 | Pertuzumab |
| 10854-11395 | K145T_V177D_S188D | S176K_Y178R | S188K | S176E_T178E | CAT-2200 | Pertuzumab |
| 10855-11312 | K145T_V177D_S188D | S176K_Y178R | L143K | V133D | CAT-2200 | Pertuzumab |
| 10855-11321 | K145T_V177D_S188D | S176K_Y178R | L143S_S186K_S188T | V133D | CAT-2200 | Pertuzumab |
| 10855-11353 | K145T_V177D_S188D | S176K_Y178R | S186K_S188T | V133D | CAT-2200 | Pertuzumab |
| 10856-11235 | K145T_V177D_S188D | S176K_Y178R | L124R | V133G_S176D | CAT-2200 | Pertuzumab |
| 10857-11243 | K145T_V177D_S188D | S176K_Y178R | L124R | V133G_S176D_T178D | CAT-2200 | Pertuzumab |
| 10858-11255 | K145T_V177D_S188D | S176K_Y178R | L124R_Q179K | V133G_S176D_T180E | CAT-2200 | Pertuzumab |
| 10858-11267 | K145T_V177D_S188D | S176K_Y178R | L124R_S186R | V133G_S176D_T180E | CAT-2200 | Pertuzumab |
| 10860-11406 | K145T_V177D_S188D | S176K_Y178R | V177I_S188K | V133I_S176D_T178E | CAT-2200 | Pertuzumab |
| 10861-11409 | K145T_V177D_S188D | S176K_Y178R | V177I_S188K | V133I_S176E_T178E | CAT-2200 | Pertuzumab |
| 10862-11414 | K145T_V177D_S188D | S176K_Y178R | V177I_S188K | V133L_S176D_T178E | CAT-2200 | Pertuzumab |
| 10869-11390 | K145T_V177T_S188E | Y178K | S188K | S176D_T178E | CAT-2200 | Pertuzumab |
| 10870-11396 | K145T_V177T_S188E | Y178K | S188K | S176E_T178E | CAT-2200 | Pertuzumab |
| 10871-11240 | K145T_V177T_S188E | Y178K | L124R | V133G_S176D | CAT-2200 | Pertuzumab |
| 10873-11256 | K145T_V177T_S188E | Y178K | L124R_Q179K | V133G_S176D_T180E | CAT-2200 | Pertuzumab |
| 10873-11268 | K145T_V177T_S188E | Y178K | L124R_S186R | V133G_S176D_T180E | CAT-2200 | Pertuzumab |
| 10875-11416 | K145T_V177T_S188E | Y178K | V177I_S188K | V133L_S176D_T178E | CAT-2200 | Pertuzumab |
| 10881-11412 | L124E_V190D | L135K_Y178F | V177I_S188K | V133L_S176D_T178E | CAT-2200 | Pertuzumab |
| 10883-11236 | L124E_V190D | V133I_L135K | L124R | V133G_S176D | CAT-2200 | Pertuzumab |
| 10888-11415 | L124E_V190D | V133I_L135K_Y178F | V177I_S188K | V133L_S176D_T178E | CAT-2200 | Pertuzumab |
| 10898-11239 | L124E_V190E | V133I_L135R_Y178F | L124R | V133G_S176D | CAT-2200 | Pertuzumab |
| 10923-11306 | L143D_K145T | E124Q_T131K | L143K | Q124E_V133D | CAT-2200 | Pertuzumab |
| 10924-11376 | L143D_K145T | E124Q_T131K | S188K | S131D_V133G_T178F | CAT-2200 | Pertuzumab |
| 10926-11391 | L143D_K145T | E124Q_T131K | S188K | S176E_T178E | CAT-2200 | Pertuzumab |
| 10927-11290 | L143D_K145T | E124Q_T131K | L143I_S186K | V133D | CAT-2200 | Pertuzumab |
| 10927-11344 | L143D_K145T | E124Q_T131K | S186K | V133D | CAT-2200 | Pertuzumab |
| 10928-11294 | L143D_K145T | E124Q_T131K | L143I_S186K | V133D_Q160E | CAT-2200 | Pertuzumab |
| 10928-11348 | L143D_K145T | E124Q_T131K | S186K | V133D_Q160E | CAT-2200 | Pertuzumab |
| 10929-11249 | L143D_K145T | E124Q_T131K | L124R_Q179K | V133G_S176D_T178E | CAT-2200 | Pertuzumab |
| 10929-11257 | L143D_K145T | E124Q_T131K | L124R_S186K | V133G_S176D_T178E | CAT-2200 | Pertuzumab |
| 10929-11261 | L143D_K145T | E124Q_T131K | L124R_S186R | V133G_S176D_T178E | CAT-2200 | Pertuzumab |
| 10931-11251 | L143D_K145T | E124Q_T131K | L124R_Q179K | V133G_S176D_T180E | CAT-2200 | Pertuzumab |
| 10931-11259 | L143D_K145T | E124Q_T131K | L124R_S186K | V133G_S176D_T180E | CAT-2200 | Pertuzumab |
| 10931-11263 | L143D_K145T | E124Q_T131K | L124R_S186R | V133G_S176D_T180E | CAT-2200 | Pertuzumab |
| 10934-11410 | L143D_K145T | E124Q_T131K | V177I_S188K | V133I_5176D_T178E | CAT-2200 | Pertuzumab |
| 10942-11291 | L143D_K145T | E124Q_T131K_V133S | L143I_S186K | V133D | CAT-2200 | Pertuzumab |
| 10942-11345 | L143D_K145T | E124Q_T131K_V133S | S186K | V133D | CAT-2200 | Pertuzumab |
| 10943-11295 | L143D_K145T | E124Q_T131K_V133S | L143I_S186K | V133D_Q160E | CAT-2200 | Pertuzumab |
| 10943-11349 | L143D_K145T | E124Q_T131K_V133S | S186K | V133D_Q160E | CAT-2200 | Pertuzumab |
| 10944-11307 | L143D_K145T | E124Q_T131R | L143K | Q124E_V133D | CAT-2200 | Pertuzumab |
| 10944-11314 | L143D_K145T | E124Q_T131R | L143R | Q124E_V133D | CAT-2200 | Pertuzumab |
| 10945-11377 | L143D_K145T | E124Q_T131R | S188K | S131D_V133G_T178F | CAT-2200 | Pertuzumab |
| 10946-11382 | L143D_K145T | E124Q_T131R | S188K | S131E_V133G_T178F | CAT-2200 | Pertuzumab |
| 10947-11392 | L143D_K145T | E124Q_T131R | S188K | S176E_T178E | CAT-2200 | Pertuzumab |
| 10948-11250 | L143D_K145T | E124Q_T131R | L124R_Q179K | V133G_S176D_T178E | CAT-2200 | Pertuzumab |
| 10948-11258 | L143D_K145T | E124Q_T131R | L124R_S186K | V133G_S176D_T178E | CAT-2200 | Pertuzumab |
| 10948-11262 | L143D_K145T | E124Q_T131R | L124R_S186R | V133G_S176D_T178E | CAT-2200 | Pertuzumab |
| 10950-11252 | L143D_K145T | E124Q_T131R | L124R_Q179K | V133G_S176D_T180E | CAT-2200 | Pertuzumab |
| 10950-11260 | L143D_K145T | E124Q_T131R | L124R_S186K | V133G_S176D_T180E | CAT-2200 | Pertuzumab |
| 10950-11264 | L143D_K145T | E124Q_T131R | L124R_S186R | V133G_S176D_T180E | CAT-2200 | Pertuzumab |
| 10952-11398 | L143D_K145T | E124Q_T131R | S188K | V133L_S176D_T178E | CAT-2200 | Pertuzumab |
| 10953-11411 | L143D_K145T | E124Q_T131R | V177L_S188K | V133L_S176D_T178E | CAT-2200 | Pertuzumab |
| 10962-11296 | L143D_K145T | T131K | L143I_S186K | V133D_Q160E | CAT-2200 | Pertuzumab |
| 10962-11350 | L143D_K145T | T131K | S186K | V133D_Q160E | CAT-2200 | Pertuzumab |
| 10965-11293 | L143D_K145T | T131K_V133S | L143I_S186K | V133D | CAT-2200 | Pertuzumab |
| 10965-11347 | L143D_K145T | T131K_V133S | S186K | V133D | CAT-2200 | Pertuzumab |
| 10966-11297 | L143D_K145T | T131K_V133S | L143I_S186K | V133D_Q160E | CAT-2200 | Pertuzumab |
| 10966-11351 | L143D_K145T | T131K_V133S | S186K | V133D_Q160E | CAT-2200 | Pertuzumab |
| 10969-11290 | L143D_K145T_Q179E | E124Q_T131K | L143I_S186K | V133D | CAT-2200 | Pertuzumab |
| 10969-11344 | L143D_K145T_Q179E | E124Q_T131K | S186K | V133D | CAT-2200 | Pertuzumab |
| 10970-11294 | L143D_K145T_Q179E | E124Q_T131K | L143I_S186K | V133D_Q160E | CAT-2200 | Pertuzumab |
| 10970-11348 | L143D_K145T_Q179E | E124Q_T131K | S186K | V133D_Q160E | CAT-2200 | Pertuzumab |
| 10973-11291 | L143D_K145T_Q179E | E124Q_T131K_V133S | L143I_S186K | V133D | CAT-2200 | Pertuzumab |
| 10973-11345 | L143D_K145T_Q179E | E124Q_T131K_V133S | S186K | V133D | CAT-2200 | Pertuzumab |
| 10974-11295 | L143D_K145T_Q179E | E124Q_T131K_V133S | L143I_S186K | V133D_Q160E | CAT-2200 | Pertuzumab |
| 10974-11349 | L143D_K145T_Q179E | E124Q_T131K_V133S | S186K | V133D_Q160E | CAT-2200 | Pertuzumab |
| 10977-11292 | L143D_K145T_Q179E | T131K | L143I_S186K | V133D | CAT-2200 | Pertuzumab |
| 10977-11346 | L143D_K145T_Q179E | T131K | S186K | V133D | CAT-2200 | Pertuzumab |
| 10978-11296 | L143D_K145T_Q179E | T131K | L143I_S186K | V133D_Q160E | CAT-2200 | Pertuzumab |
| 10978-11350 | L143D_K145T_Q179E | T131K | S186K | V133D_Q160E | CAT-2200 | Pertuzumab |
| 10981-11293 | L143D_K145T_Q179E | T131K_V133S | L143I_S186K | V133D | CAT-2200 | Pertuzumab |
| 10981-11347 | L143D_K145T_Q179E | T131K_V133S | S186K | V133D | CAT-2200 | Pertuzumab |
| 10982-11297 | L143D_K145T_Q179E | T131K_V133S | L143I_S186K | V133D_Q160E | CAT-2200 | Pertuzumab |
| 10982-11351 | L143D_K145T_Q179E | T131K_V133S | S186K | V133D_Q160E | CAT-2200 | Pertuzumab |
| 10983-11306 | L143D_K145T_Q179E | E124Q_T131K | L143K | Q124E_V133D | CAT-2200 | Pertuzumab |
| 10984-11376 | L143D_K145T_Q179E | E124Q_T131K | S188K | S131D_V133G_T178F | CAT-2200 | Pertuzumab |
| 10985-11381 | L143D_K145T_Q179E | E124Q_T131K | S188K | S131E_V133G_T178F | CAT-2200 | Pertuzumab |
| 10986-11391 | L143D_K145T_Q179E | E124Q_T131K | S188K | S176E_T178E | CAT-2200 | Pertuzumab |
| 10987-11249 | L143D_K145T_Q179E | E124Q_T131K | L124R_Q179K | V133G_S176D_T178E | CAT-2200 | Pertuzumab |
| 10987-11257 | L143D_K145T_Q179E | E124Q_T131K | L124R_S186K | V133G_S176D_T178E | CAT-2200 | Pertuzumab |
| 10987-11261 | L143D_K145T_Q179E | E124Q_T131K | L124R_S186R | V133G_S176D_T178E | CAT-2200 | Pertuzumab |
| 10989-11251 | L143D_K145T_Q179E | E124Q_T131K | L124R_Q179K | V133G_S176D_T180E | CAT-2200 | Pertuzumab |
| 10989-11259 | L143D_K145T_Q179E | E124Q_T131K | L124R_S186K | V133G_S176D_T180E | CAT-2200 | Pertuzumab |
| 10989-11263 | L143D_K145T_Q179E | E124Q_T131K | L124R_S186R | V133G_S176D_T180E | CAT-2200 | Pertuzumab |
| 10992-11410 | L143D_K145T_Q179E | E124Q_T131K | V177I_5188K | V133I_S176D_T178E | CAT-2200 | Pertuzumab |
| 10998-11307 | L143D_K145T_Q179E | E124Q_T131R | L143K | Q124E_V133D | CAT-2200 | Pertuzumab |
| 10998-11314 | L143D_K145T_Q179E | E124Q_T131R | L143R | Q124E_V133D | CAT-2200 | Pertuzumab |
| 10999-11377 | L143D_K145T_Q179E | E124Q_T131R | S188K | S131D_V133G_T178F | CAT-2200 | Pertuzumab |
| 11000-11382 | L143D_K145T_Q179E | E124Q_T131R | S188K | S131E_V133G_T178F | CAT-2200 | Pertuzumab |
| 11001-11392 | L143D_K145T_Q179E | E124Q_T131R | S188K | S176E_T178E | CAT-2200 | Pertuzumab |
| 11002-11250 | L143D_K145T_Q179E | E124Q_T131R | L124R_Q179K | V133G_S176D_T178E | CAT-2200 | Pertuzumab |
| 11002-11258 | L143D_K145T_Q179E | E124Q_T131R | L124R_S186K | V133G_S176D_T178E | CAT-2200 | Pertuzumab |
| 11002-11262 | L143D_K145T_Q179E | E124Q_T131R | L124R_S186R | V133G_S176D_T178E | CAT-2200 | Pertuzumab |
| 11004-11252 | L143D_K145T_Q179E | E124Q_T131R | L124R_Q179K | V133G_S176D_T180E | CAT-2200 | Pertuzumab |
| 11004-11260 | L143D_K145T_Q179E | E124Q_T131R | L124R_S186K | V133G_S176D_T180E | CAT-2200 | Pertuzumab |
| 11004-11264 | L143D_K145T_Q179E | E124Q_T131R | L124R_S186R | V133G_S176D_T180E | CAT-2200 | Pertuzumab |
| 11006-11398 | L143D_K145T_Q179E | E124Q_T131R | S188K | V133I_S176D_T178E | CAT-2200 | Pertuzumab |
| 11007-11411 | L143D_K145T_Q179E | E124Q_T131R | V177I_S188K | V133I_S176D_T178E | CAT-2200 | Pertuzumab |
| 11025-11284 | L143K | K129T_V133D | L143E_K145T_Q179E_S188L | Q124K_T178R | CAT-2200 | Pertuzumab |
| 11026-11270 | L143K | K129T_V133D | L124W_L143E_K145T_Q179E | Q124K_V133A_T178R | CAT-2200 | Pertuzumab |
| 11027-11281 | L143K | K129T_V133D | L143E_K145T_Q179E_ | Q124R_Q160R_S176A_ | CAT-2200 | Pertuzumab |
| | | | S186I_S188W | T178R | | |
| 11027-11287 | L143K | K129T_V133D | L143E_K145T_Q179E_S188W | Q124R_Q160R_S176A_ | CAT-2200 | Pertuzumab |
| | | | | T178R | | |
| 11028-11275 | L143K | K129T_V133D | L143E_K145T_Q179E | Q124R_Q160R_T178R | CAT-2200 | Pertuzumab |
| 11029-11282 | L143K | K129T_V133D | L143E_K145T_Q179E_ | Q124R_S176A_T178R | CAT-2200 | Pertuzumab |
| | | | S186I_S188W | | | |
| 11029-11288 | L143K | K129T_V133D | L143E_K145T_Q179E_S188W | Q124R_S176A_T178R | CAT-2200 | Pertuzumab |
| 11030-11276 | L143K | K129T_V133D | L143E_K145T_Q179E | Q124R_T178R | CAT-2200 | Pertuzumab |
| 11030-11285 | L143K | K129T_V133D | L143E_K145T_Q179E_S188L | Q124R_T178R | CAT-2200 | Pertuzumab |
| 11032-11199 | L143K | K129T_V133D | L124E_K145T_Q179E | S131K_V133G_S176R | CAT-2200 | Pertuzumab |
| 11033-11209 | L143K | K129T_V133D | L124E_K145T_Q179E_ | S131K_V133G_S176R_ | CAT-2200 | Pertuzumab |
| | | | S186I_S188W | T178A | | |
| 11033-11215 | L143K | K129T_V133D | L124E_K145T_Q179E_S188W | S131K_V133G_S176R_ | CAT-2200 | Pertuzumab |
| | | | | T178A | | |
| 11034-11204 | L143K | K129T_V133D | L124E_K145T_Q179E | S131R_V133G_S176R | CAT-2200 | Pertuzumab |
| 11035-11212 | L143K | K129T_V133D | L124E_K145T_Q179E_ | S131R_V133G_S176R_ | CAT-2200 | Pertuzumab |
| | | | S186I_S188W | T178A | | |
| 11035-11218 | L143K | K129T_V133D | L124E_K145T_Q179E_S188W | S131R_V133G_S176R_ | CAT-2200 | Pertuzumab |
| | | | | T178A | | |
| 11036-11221 | L143K | K129T_V133D | L124E_K145T_S186E | S131R_V1335_L135K | CAT-2200 | Pertuzumab |
| 11039-11224 | L143K | K129T_V133D | L124E_L143E_K145T | S131R_V133T_L135K_ | CAT-2200 | Pertuzumab |
| | | | | T178S | | |
| 11042-11273 | L143S_S186K_S188T | V133D_Y178T | L143E_K145T_Q179E | Q124R_Q160K_T178R | CAT-2200 | Pertuzumab |
| 11043-11181 | L143S_S186K_S188T | V133D_Y178T | K145T_V177D_S188D | S176K_T178K | CAT-2200 | Pertuzumab |
| 11044-11191 | L143S_S186K_S188T | V133D_Y178T | K145T_V177D_S188D | S176K_T178R | CAT-2200 | Pertuzumab |
| 11055-11322 | S186K_S188T | T131S_V133D_Y178T | L143T_S188D | S131K_S176A_T178S | CAT-2200 | Pertuzumab |
| 11056-11324 | S186K_S188T | T131S_V133D_Y178T | L143T_S188D | S131K_T178S | CAT-2200 | Pertuzumab |
| 11061-11273 | S186K_S188T | V133D_Y178T | L143E_K145T_Q179E | Q124R_Q160K_T178R | CAT-2200 | Pertuzumab |
| 11062-11323 | S186K_S188T | V133D_Y178T | L143T_S188D | S131K_S176A_T178S | CAT-2200 | Pertuzumab |
| 11063-11325 | S186K_S188T | V133D_Y178T | L143T_S188D | S131K_T178S | CAT-2200 | Pertuzumab |
| 11064-11201 | S186K_S188T | V133D_Y178T | L124E_K145T_Q179E | S131K_V133G_S176R | CAT-2200 | Pertuzumab |
| 11065-11206 | S186K_S188T | V133D_Y178T | L124E_K145T_Q179E | S131R_V133G_S176R | CAT-2200 | Pertuzumab |
| 11066-11181 | S186K_S188T | V133D_Y178T | K145T_V177D_S188D | S176K_T178K | CAT-2200 | Pertuzumab |
| 11076-11355 | S188A | S176A_Y178W | S186L_S188W | S176A_T178A | CAT-2200 | Pertuzumab |
| 11087-11179 | S188K | S176D_Y178E | K145T_V177D_S188D | S176K_T178K | CAT-2200 | Pertuzumab |
| 11088-11185 | S188K | S176D_Y178E | K145T_V177D_S188D | S176K_T178L | CAT-2200 | Pertuzumab |
| 11089-11189 | S188K | S176D_Y178E | K145T_V177D_S188D | S176K_T178R | CAT-2200 | Pertuzumab |
| 11096-11272 | S188K | S176E_Y178E | L143E_K145T_Q179E | Q124R_Q160K_T178R | CAT-2200 | Pertuzumab |
| 11097-11277 | S188K | S176E_Y178E | L143E_K145T_Q179E | Q124R_T178R | CAT-2200 | Pertuzumab |
| 11098-11200 | S188K | S176E_Y178E | L124E_K145T_Q179E | S131K_V133G_S176R | CAT-2200 | Pertuzumab |
| 11099-11210 | S188K | S176E_Y178E | L124E_K145T_Q179E_S186I_ | S131K_V133G_S176R_ | CAT-2200 | Pertuzumab |
| | | | S188W | T178A | | |
| 11099-11216 | S188K | S176E_Y178E | L124E_K145T_Q179E_S188W | S131K_V133G_S176R_ | CAT-2200 | Pertuzumab |
| | | | | T178A | | |
| 11100-11205 | S188K | S176E_Y178E | L124E_K145T_Q179E | S131R_V133G_S176R | CAT-2200 | Pertuzumab |
| 11101-11213 | S188K | S176E_Y178E | L124E_K145T_Q179E_S186I_ | S131R_V133G_S176R_ | CAT-2200 | Pertuzumab |
| | | | S188W | T178A | | |
| 11101-11219 | S188K | S176E_Y178E | L124E_K145T_Q179E_S188W | S131R_V133G_S176R_ | CAT-2200 | Pertuzumab |
| | | | | T178A | | |
| 11102-11180 | S188K | S176E_Y178E | K145T_V177D_S188D | S176K_T178K | CAT-2200 | Pertuzumab |
| 11103-11186 | S188K | S176E_Y178E | K145T_V177D_S188D | S176K_T178L | CAT-2200 | Pertuzumab |
| 11104-11190 | S188K | S176E_Y178E | K145T_V177D_S188D | S176K_T178R | CAT-2200 | Pertuzumab |
| 11105-11195 | S188K | S176E_Y178E | L124E | V133A_S176K | CAT-2200 | Pertuzumab |
| 11106-11225 | S188K | S176E_Y178E | L124E_S186I_S188W | V133A_S176K_T178A | CAT-2200 | Pertuzumab |
| 11106-11229 | S188K | S176E_Y178E | L124E_S188W | V133A_S176K_T178A | CAT-2200 | Pertuzumab |
| 11107-11197 | S188K | S176E_Y178E | L124E | V133G_S176R | CAT-2200 | Pertuzumab |
| 11108-11227 | S188K | S176E_Y178E | L124E_S186I_S188W | V133G_S176R_T178A | CAT-2200 | Pertuzumab |
| 11108-11231 | S188K | S176E_Y178E | L124E_S188W | V133G_S176R_T178A | CAT-2200 | Pertuzumab |
| 11124-11202 | S188K | V133I_S176D_Y178E | L124E_K145T_Q179E | S131K_V133G_S176R | CAT-2200 | Pertuzumab |
| 11125-11207 | S188K | V133I_S176D_Y178E | L124E_K145T_Q179E | S131R_V133G_S176R | CAT-2200 | Pertuzumab |
| 11140-11202 | V177I_S188K | V133I_S176D_Y178E | L124E_K145T_Q179E | S131K_V133G_S176R | CAT-2200 | Pertuzumab |
| 11141-11207 | V177I_S188K | V133I_S176D_Y178E | L124E_K145T_Q179E | S131R_V133G_S176R | CAT-2200 | Pertuzumab |
| 11142-11182 | V177I_S188K | V133I_S176D_Y178E | K145T_V177D_S188D | S176K_T178K | CAT-2200 | Pertuzumab |
| 11143-11187 | V177I_S188K | V133I_S176D_Y178E | K145T_V177D_S188D | S176K_T178L | CAT-2200 | Pertuzumab |
| 11144-11192 | V177I_S188K | V133I_S176D_Y178E | K145T_V177D_S188D | S176K_T178R | CAT-2200 | Pertuzumab |
| 11148-11183 | V177I_S188K | V133I_S176D_Y178E | K145T_V177D_S188D | S176K_T178K | CAT-2200 | Pertuzumab |
| 11149-11188 | V177I_S188K | V133I_S176D_Y178E | K145T_V177D_S188D | S176K_T178L | CAT-2200 | Pertuzumab |
| 11150-11193 | V177I_S188K | V133I_S176D_Y178E | K145T_V177D_S188D | S176K_T178R | CAT-2200 | Pertuzumab |
| 11156-11274 | V177I_S188K | V133I_S176D_Y178E | L143E_K145T_Q179E | Q124R_Q160K_T178R | CAT-2200 | Pertuzumab |
| 11157-11279 | V177I_S188K | V133I_S176D_Y178E | L143E_K145T_Q179E | Q124R_T178R | CAT-2200 | Pertuzumab |
| 11158-11203 | V177I_S188K | V133I_S176D_Y178E | L124E_K145T_Q179E | S131K_V133G_S176R | CAT-2200 | Pertuzumab |
| 11159-11211 | V177I_S188K | V133L_S176D_Y178E | L124E_K145T_Q179E_S186I_ | S131K_V133G_S176R_ | CAT-2200 | Pertuzumab |
| | | | S188W | T178A | | |
| 11159-11217 | V177I_S188K | V133L_S176D_Y178E | L124E_K145T_Q179E_S188W | S131K_V133G_S176R_ | CAT-2200 | Pertuzumab |
| | | | | T178A | | |
| 11160-11208 | V177I_S188K | V133L_S176D_Y178E | L124E_K145T_Q179E | S131R_V133G_S176R | CAT-2200 | Pertuzumab |
| 11161-11214 | V177I_S188K | V133L_S176D_Y178E | L124E_K145T_Q179E_S186I_ | S131R_V133G_S176R_ | CAT-2200 | Pertuzumab |
| | | | S188W | T178A | | |
| 11161-11220 | V177I_S188K | V133L_S176D_Y178E | L124E_K145T_Q179E_S188W | S131R_V133G_S176R_ | CAT-2200 | Pertuzumab |
| | | | | T178A | | |
| 11162-11184 | V177I_S188K | V133L_S176D_Y178E | K145T_V177D_S188D | S176K_T178K | CAT-2200 | Pertuzumab |
| 11163-11194 | V177I_S188K | V133L_S176D_Y178E | K145T_V177D_S188D | S176K_T178R | CAT-2200 | Pertuzumab |
| 11164-11196 | V177I_S188K | V133L_S176D_Y178E | L124E | V133A_S176K | CAT-2200 | Pertuzumab |
| 11165-11226 | V177I_S188K | V133L_S176D_Y178E | L124E_S186I_S188W | V133A_S176K_T178A | CAT-2200 | Pertuzumab |
| 11165-11230 | V177I_S188K | V133L_S176D_Y178E | L124E_S188W | V133A_S176K_T178A | CAT-2200 | Pertuzumab |
| 11166-11198 | V177I_S188K | V133L_S176D_Y178E | L124E | V133G_S176R | CAT-2200 | Pertuzumab |
| 11167-11228 | V177I_S188K | V133L_S176D_Y178E | L124E_S186I_S188W | V133G_S176R_T178A | CAT-2200 | Pertuzumab |
| 11167-11232 | V177I_S188K | V133L_S176D_Y178E | L124E_S188W | V133G_S176R_T178A | CAT-2200 | Pertuzumab |

TABLE 4B
K-K derived K-L designs libarary

| Unique identifier | H1 mutation (Kabat) | L1 mutation (Kabat) | H2 mutation (Kabat) | L2 mutation (Kabat) | H1L1 Fab | H2L2 Fab |
|---|---|---|---|---|---|---|
| 10629-10695 | A139W | T116A | — | L135W | CAT-2200 | Pertuzumab |
| 10631-10722 | D146G_Q179K | S180E | L143E_K145T | Q124R | CAT-2200 | Pertuzumab |
| 10632-10724 | D146G_Q179K | S180E | L143E_K145T | Q124R_Q160K_T178R | CAT-2200 | Pertuzumab |
| 10640-10713* | L124E | V133G_S176R | L124R | V133G_S176D | CAT-2200 | Pertuzumab |
| 10652-10734 | L143E_K145T | E124R | Q179K | Q124E_Q160E_T180E | CAT-2200 | Pertuzumab |
| 10652-10755 | L143E_K145T | E124R | S186R | Q124E_Q160E_T180E | CAT-2200 | Pertuzumab |
| 10656-10758 | L143E_K145T_Q179E | E124K_Y178R | S186R | Q160E_T180E | CAT-2200 | Pertuzumab |
| 10657-10760 | L143E_K145T_Q179E | E124K_Y178R | S186R | T178E_T180E | CAT-2200 | Pertuzumab |
| 10662-10733* | L45F | — | L45P | P44F | CAT-2200 | Pertuzumab |
| 10671-10751 | Q39D | Q38R | Q39R | Q38E | CAT-2200 | Pertuzumab |
| 10674-10749* | Q39E | Q38K | Q39K | Q38E | CAT-2200 | Pertuzumab |
| 10676-10750* | Q39E | Q38R | Q39R | Q38D | CAT-2200 | Pertuzumab |
| 10677-10751 | Q39E | Q38R | Q39R | Q38E | CAT-2200 | Pertuzumab |
| 10624-10700* | — | L135W | A139W | F116A | CAT-2200 | Pertuzumab |
| 10625-10701* | — | L135W | A139W | F116A_L135V | CAT-2200 | Pertuzumab |
| 10652-10702 | L143E_K145T | E124R | D146G_Q179K | Q124E_Q160E_T180E | CAT-2200 | Pertuzumab |
| 10684-10706 | S186R | K129T_S180E | K145T_Q179E | S131K | CAT-2200 | Pertuzumab |
| 10689-10707 | S186R | S180E | K145T_S179E | S131K | CAT-2200 | Pertuzumab |
| 10682-10718 | S186R | — | L143E_K145T | Q124K_T178R | CAT-2200 | Pertuzumab |
| 10685-10719 | S186R | S180E | L143E_K145T | Q124K_T178R | CAT-2200 | Pertuzumab |
| 10658-10720 | L143K | — | L143E_K145T | Q124R | CAT-2200 | Pertuzumab |
| 10659-10720 | L143R | — | L143E_K145T | Q124R | CAT-2200 | Pertuzumab |
| 10683-10720 | S186R | — | L143E_K145T | Q124R | CAT-2200 | Pertuzumab |
| 10664-10722 | Q179K | S180E | L143E_K145T | Q124R | CAT-2200 | Pertuzumab |
| 10687-10722 | S186R | S180E | L143E_K145T | Q124R | CAT-2200 | Pertuzumab |
| 10665-10724 | Q179K | S180E | L143E_K145T | Q124R_Q160K_T178R | CAT-2200 | Pertuzumab |
| 10688-10724 | S186R | S180E | L143E_K145T | Q124R_Q160K_T178R | CAT-2200 | Pertuzumab |
| 10685-10726 | S186R | S180E | L143E_K145T_Q179E | Q124K_T178R | CAT-2200 | Pertuzumab |
| 10690-10727 | S186R | Y178E_S180E | L143E_K145T_Q179E | Q124K_T178R | CAT-2200 | Pertuzumab |
| 10621-10731* | — | — | L45A | P44F | CAT-2200 | Pertuzumab |
| 10662-10731* | L45F | — | L45A | P44F | CAT-2200 | Pertuzumab |
| 10621-10733* | — | — | L45P | P44F | CAT-2200 | Pertuzumab |
| 10622-10736 | — | — | Q39D | Q38K | CAT-2200 | Pertuzumab |
| 10678-10737 | Q39K | Q38D | Q39D | Q38K | CAT-2200 | Pertuzumab |
| 10679-10738 | Q39K | Q38E | Q39D | Q38K | CAT-2200 | Pertuzumab |
| 10623-10739 | — | — | Q39D | Q38R | CAT-2200 | Pertuzumab |
| 10680-10740* | Q39R | Q38D | Q39D | Q38R | CAT-2200 | Pertuzumab |
| 10681-10741 | Q39R | Q38E | Q39D | Q38R | CAT-2200 | Pertuzumab |
| 10622-10742 | — | — | Q39E | Q38K | CAT-2200 | Pertuzumab |
| 10678-10743 | Q39K | Q38D | Q39E | Q38K | CAT-2200 | Pertuzumab |
| 10679-10744 | Q39K | Q38E | Q39E | Q38K | CAT-2200 | Pertuzumab |
| 10623-10745 | — | — | Q39E | Q38R | CAT-2200 | Pertuzumab |
| 10680-10746 | Q39R | Q38D | Q39E | Q38R | CAT-2200 | Pertuzumab |
| 10681-10747 | Q39R | Q38E | Q39E | Q38R | CAT-2200 | Pertuzumab |

*Designs with exact match mutations to kappa-kappa library designs.

TABLE 5A
LCCA results for K-L designs that passed the performance cut-off criteria

| | Column 2 | Column 3 | | Column 5 | Column 6 | | | |
|---|---|---|---|---|---|---|---|---|
| | H1L1:H1L2 | H1L1:H1L2 range | Column 4 | H2L2:H2L1 | H2L2:H2L1 range | Column 7 | | |
| | normalized | of normalized | H1L1:H1L2 | normalized | of normalized | H2L2:H2L1 | | |
| Column 1 | median scalar | scalar value | normalized | median scalar | scalar value | normalized | Column 8 | Column 9 |
| Unique identifier | value In(r1/f1) | In(r1/f1) | median ratio | value In(r1/f1) | In(r1/f1) | median ratio | H1L1 Fab | H2L2 Fab |
| 10771-11335 | 0.48 | NA | 62:38 | −0.39 | NA | 40:60 | CAT-2200 | Pertuzumab |
| 10771-11360 | 0.48 | NA | 62:38 | −0.39 | NA | 40:60 | CAT-2200 | Pertuzumab |
| 10776-11312 | −0.19 | NA | 45:55 | 1.39 | 0.1 | 80:20 | CAT-2200 | Pertuzumab |
| 10776-11321 | −0.19 | NA | 45:55 | 0.54 | NA | 63:37 | CAT-2200 | Pertuzumab |
| 10776-11353 | −0.19 | NA | 45:55 | 0.85 | 0.03 | 70:30 | CAT-2200 | Pertuzumab |
| 10780-11417 | 0.03 | NA | 51:49 | 0.6 | 4.25 | 65:35 | CAT-2200 | Pertuzumab |
| 10781-11300 | 0.74 | NA | 68:32 | −0.16 | NA | 46:54 | CAT-2200 | Pertuzumab |
| 10781-11419 | 0.74 | NA | 68:32 | −0.34 | NA | 42:58 | CAT-2200 | Pertuzumab |
| 10786-11299 | −0.07 | NA | 48:52 | 0.49 | 0.43 | 62:38 | CAT-2200 | Pertuzumab |
| 10786-11418 | −0.07 | NA | 48:52 | 0.47 | NA | 62:38 | CAT-2200 | Pertuzumab |
| 10787-11301 | 1.15 | 0.37 | 76:24 | −0.04 | NA | 49:51 | CAT-2200 | Pertuzumab |
| 10787-11420 | 1.15 | 0.37 | 76:24 | −0.26 | NA | 44:56 | CAT-2200 | Pertuzumab |
| 10788-11422 | −0.01 | NA | 50:50 | 0.41 | NA | 60:40 | CAT-2200 | Pertuzumab |
| 10792-11305 | 0.04 | NA | 51:49 | 0.62 | NA | 65:35 | CAT-2200 | Pertuzumab |
| 10792-11424 | 0.04 | NA | 51:49 | 0.9 | NA | 71:29 | CAT-2200 | Pertuzumab |
| 10793-11375 | 0.24 | 0.14 | 56:44 | 0.93 | 0.18 | 72:28 | CAT-2200 | Pertuzumab |
| 10795-11390 | 1.27 | 0.55 | 78:22 | 1.41 | NA | 80:20 | CAT-2200 | Pertuzumab |
| 10796-11396 | 0.81 | 0.2 | 69:31 | 2.67 | NA | 94:6 | CAT-2200 | Pertuzumab |
| 10798-11256 | 2.12 | 0.81 | 89:11 | 0.63 | 0.12 | 65:35 | CAT-2200 | Pertuzumab |
| 10798-11268 | 2.12 | 0.81 | 89:11 | 1.21 | 0.31 | 77:23 | CAT-2200 | Pertuzumab |
| 10800-11416 | 1.12 | 0.42 | 75:25 | 0.55 | NA | 63:37 | CAT-2200 | Pertuzumab |
| 10806-11326 | 0.39 | 0.61 | 60:40 | 2.33 | 0.86 | 91:9 | CAT-2200 | Pertuzumab |
| 10806-11369 | 0.39 | 0.61 | 60:40 | 3.29 | 0.39 | 96:4 | CAT-2200 | Pertuzumab |
| 10808-11308 | 0.59 | NA | 64:36 | 1.78 | NA | 86:14 | CAT-2200 | Pertuzumab |
| 10808-11315 | 0.59 | NA | 64:36 | 1.3 | 0.22 | 79:21 | CAT-2200 | Pertuzumab |
| 10809-11378 | 1.29 | 0.73 | 78:22 | 1.85 | 0.12 | 86:14 | CAT-2200 | Pertuzumab |
| 10810-11383 | 0.8 | 0.22 | 69:31 | 2.77 | 0.55 | 94:6 | CAT-2200 | Pertuzumab |
| 10811-11386 | 1.71 | 0.38 | 85:15 | 1.83 | NA | 86:14 | CAT-2200 | Pertuzumab |
| 10812-11393 | 0.56 | 0.54 | 64:36 | 2.75 | 0.48 | 94:6 | CAT-2200 | Pertuzumab |
| 10813-11311 | 0.39 | NA | 60:40 | 1.2 | NA | 77:23 | CAT-2200 | Pertuzumab |
| 10813-11320 | 0.39 | NA | 60:40 | 0.58 | NA | 64:36 | CAT-2200 | Pertuzumab |
| 10813-11352 | 0.39 | NA | 60:40 | 0.49 | NA | 62:38 | CAT-2200 | Pertuzumab |
| 10814-11233 | 1.53 | 0.78 | 82:18 | 1.63 | 0.4 | 84:16 | CAT-2200 | Pertuzumab |
| 10816-11253 | 1.95 | 0.27 | 88:12 | 0.75 | 0.08 | 68:32 | CAT-2200 | Pertuzumab |
| 10816-11265 | 1.95 | 0.27 | 88:12 | 1.06 | 0.14 | 74:26 | CAT-2200 | Pertuzumab |
| 10818-11404 | 1.14 | 0.77 | 76:24 | 2.34 | NA | 91:9 | CAT-2200 | Pertuzumab |
| 10819-11407 | 0.35 | 0.46 | 59:41 | 2.83 | NA | 94:6 | CAT-2200 | Pertuzumab |
| 10820-11413 | 1.61 | 0.48 | 83:17 | 2.37 | NA | 91:9 | CAT-2200 | Pertuzumab |
| 10827-11373 | −0.02 | NA | 50:50 | 2.64 | NA | 93:7 | CAT-2200 | Pertuzumab |
| 10828-11327 | −0.35 | 0.61 | 41:59 | 1.88 | 0.41 | 87:13 | CAT-2200 | Pertuzumab |
| 10828-11370 | −0.35 | 0.61 | 41:59 | 2.07 | NA | 89:11 | CAT-2200 | Pertuzumab |
| 10830-11309 | 0.31 | NA | 58:42 | 2.64 | 0.73 | 93:7 | CAT-2200 | Pertuzumab |
| 10830-11316 | 0.31 | NA | 58:42 | 1.53 | NA | 82:18 | CAT-2200 | Pertuzumab |
| 10831-11379 | 0.71 | 0.56 | 67:33 | 1.77 | NA | 85:15 | CAT-2200 | Pertuzumab |
| 10832-11384 | 0.09 | 0.31 | 52:48 | 2.17 | 0.12 | 90:10 | CAT-2200 | Pertuzumab |
| 10833-11387 | 1.23 | 0.01 | 77:23 | 1.78 | NA | 86:14 | CAT-2200 | Pertuzumab |
| 10834-11394 | −0.29 | 0.48 | 43:57 | 2.96 | NA | 95:5 | CAT-2200 | Pertuzumab |
| 10835-11234 | 0.52 | NA | 63:37 | 1.06 | 0.39 | 74:26 | CAT-2200 | Pertuzumab |
| 10837-11254 | 1.5 | 0.38 | 82:18 | 0.67 | 0.19 | 66:34 | CAT-2200 | Pertuzumab |
| 10837-11266 | 1.5 | 0.38 | 82:18 | 0.98 | 0.09 | 73:27 | CAT-2200 | Pertuzumab |
| 10840-11408 | −0.38 | 0.47 | 41:59 | 2.79 | NA | 94:6 | CAT-2200 | Pertuzumab |
| 10848-11328 | −0.02 | 0.67 | 50:50 | 3.21 | 0.9 | 96:4 | CAT-2200 | Pertuzumab |
| 10848-11371 | −0.02 | 0.67 | 50:50 | 3.46 | 0.75 | 97:3 | CAT-2200 | Pertuzumab |
| 10850-11310 | 0.74 | 0.17 | 68:32 | 1.95 | 0.04 | 88:12 | CAT-2200 | Pertuzumab |
| 10850-11317 | 0.74 | 0.17 | 68:32 | 1.84 | 0.12 | 86:14 | CAT-2200 | Pertuzumab |
| 10851-11380 | 0.95 | 0.27 | 72:28 | 2.09 | 0.61 | 89:11 | CAT-2200 | Pertuzumab |
| 10852-11385 | 0.24 | 0.67 | 56:44 | 2.35 | 0.38 | 91:9 | CAT-2200 | Pertuzumab |
| 10853-11388 | 0.79 | 1 | 69:31 | 2.16 | NA | 90:10 | CAT-2200 | Pertuzumab |
| 10854-11395 | 0.1 | 0.15 | 52:48 | 2.87 | NA | 95:5 | CAT-2200 | Pertuzumab |
| 10855-11312 | 0.62 | 0.2 | 65:35 | 1.39 | 0.1 | 80:20 | CAT-2200 | Pertuzumab |
| 10855-11321 | 0.62 | 0.2 | 65:35 | 0.54 | NA | 63:37 | CAT-2200 | Pertuzumab |
| 10855-11353 | 0.62 | 0.2 | 65:35 | 0.85 | 0.03 | 70:30 | CAT-2200 | Pertuzumab |
| 10856-11235 | 1.24 | 0.26 | 78:22 | 1.75 | 0.78 | 85:15 | CAT-2200 | Pertuzumab |
| 10857-11243 | 0.43 | NA | 61:39 | 0.43 | 0.31 | 61:39 | CAT-2200 | Pertuzumab |
| 10858-11255 | 1.13 | 1.23 | 76:24 | 0.79 | 0.16 | 69:31 | CAT-2200 | Pertuzumab |
| 10858-11267 | 1.13 | 1.23 | 76:24 | 1.17 | 0.07 | 76:24 | CAT-2200 | Pertuzumab |
| 10860-11406 | 0.81 | 0.63 | 69:31 | 2.58 | NA | 93:7 | CAT-2200 | Pertuzumab |
| 10861-11409 | 0.18 | 0.16 | 54:46 | 2.63 | NA | 93:7 | CAT-2200 | Pertuzumab |
| 10862-11414 | 0.45 | 1.18 | 61:39 | 2.38 | NA | 92:8 | CAT-2200 | Pertuzumab |
| 10869-11390 | 1.07 | 0.67 | 74:26 | 1.41 | NA | 80:20 | CAT-2200 | Pertuzumab |
| 10870-11396 | 0.75 | 0.36 | 68:32 | 2.67 | NA | 94:6 | CAT-2200 | Pertuzumab |
| 10871-11240 | 0.87 | NA | 70:30 | 1.68 | 0.56 | 84:16 | CAT-2200 | Pertuzumab |
| 10873-11256 | 1.32 | 0.02 | 79:21 | 0.63 | 0.12 | 65:35 | CAT-2200 | Pertuzumab |
| 10873-11268 | 1.32 | 0.02 | 79:21 | 1.21 | 0.31 | 77:23 | CAT-2200 | Pertuzumab |
| 10875-11416 | 1.04 | 0.63 | 74:26 | 0.55 | NA | 63:37 | CAT-2200 | Pertuzumab |
| 10881-11412 | 0.39 | NA | 60:40 | 1.64 | NA | 84:16 | CAT-2200 | Pertuzumab |
| 10883-11236 | −0.25 | 0.64 | 44:56 | 1.8 | 1.06 | 86:14 | CAT-2200 | Pertuzumab |
| 10888-11415 | 0.27 | 0.28 | 57:43 | 1.83 | 0.08 | 86:14 | CAT-2200 | Pertuzumab |
| 10898-11239 | −0.14 | 0.52 | 47:53 | 1.56 | 0.38 | 83:17 | CAT-2200 | Pertuzumab |
| 10923-11306 | 1.11 | 0.12 | 75:25 | 2.48 | 0.22 | 92:8 | CAT-2200 | Pertuzumab |
| 10924-11376 | 0.51 | 0.14 | 62:38 | 1.5 | 0.39 | 82:18 | CAT-2200 | Pertuzumab |
| 10926-11391 | 0.21 | 0.45 | 55:45 | 2.71 | 0.33 | 94:6 | CAT-2200 | Pertuzumab |
| 10927-11290 | 0.72 | 0.39 | 67:33 | 0.36 | NA | 59:41 | CAT-2200 | Pertuzumab |
| 10927-11344 | 0.72 | 0.39 | 67:33 | 0.22 | NA | 55:45 | CAT-2200 | Pertuzumab |
| 10928-11294 | 1.23 | 0.36 | 77:23 | 0.81 | NA | 69:31 | CAT-2200 | Pertuzumab |
| 10928-11348 | 1.23 | 0.36 | 77:23 | 0.41 | NA | 60:40 | CAT-2200 | Pertuzumab |
| 10929-11249 | 1.46 | 0.91 | 81:19 | 0.77 | 0.24 | 68:32 | CAT-2200 | Pertuzumab |
| 10929-11257 | 1.46 | 0.91 | 81:19 | 1.12 | 0.28 | 75:25 | CAT-2200 | Pertuzumab |
| 10929-11261 | 1.46 | 0.91 | 81:19 | 1.04 | 0.26 | 74:26 | CAT-2200 | Pertuzumab |
| 10931-11251 | 1.17 | 0.41 | 76:24 | 0.95 | NA | 72:28 | CAT-2200 | Pertuzumab |
| 10931-11259 | 1.17 | 0.41 | 76:24 | 1.47 | NA | 81:19 | CAT-2200 | Pertuzumab |
| 10931-11263 | 1.17 | 0.41 | 76:24 | 1.5 | 0.7 | 82:18 | CAT-2200 | Pertuzumab |
| 10934-11410 | 1.23 | 0.34 | 77:23 | 2.15 | NA | 90:10 | CAT-2200 | Pertuzumab |
| 10942-11291 | 0.83 | 0.77 | 70:30 | 1.18 | NA | 76:24 | CAT-2200 | Pertuzumab |
| 10942-11345 | 0.83 | 0.77 | 70:30 | 0.82 | NA | 69:31 | CAT-2200 | Pertuzumab |
| 10943-11295 | 1.02 | 0.85 | 73:27 | 1.6 | NA | 83:17 | CAT-2200 | Pertuzumab |
| 10943-11349 | 1.02 | 0.85 | 73:27 | 1.27 | 0.16 | 78:22 | CAT-2200 | Pertuzumab |
| 10944-11307 | 1.02 | NA | 73:27 | 2.3 | 0.17 | 91:9 | CAT-2200 | Pertuzumab |
| 10944-11314 | 1.02 | NA | 73:27 | 3.06 | 0.45 | 96:4 | CAT-2200 | Pertuzumab |
| 10945-11377 | 0.97 | 0.39 | 73:27 | 2.22 | 0.65 | 90:10 | CAT-2200 | Pertuzumab |
| 10946-11382 | 0.53 | 0.18 | 63:37 | 1.67 | NA | 84:16 | CAT-2200 | Pertuzumab |
| 10947-11392 | 0.59 | 0.66 | 64:36 | 2.75 | 0.27 | 94:6 | CAT-2200 | Pertuzumab |
| 10948-11250 | 1.4 | 0.35 | 80:20 | 0.69 | NA | 67:33 | CAT-2200 | Pertuzumab |
| 10948-11258 | 1.4 | 0.35 | 80:20 | 1.22 | NA | 77:23 | CAT-2200 | Pertuzumab |
| 10948-11262 | 1.4 | 0.35 | 80:20 | 1.18 | 0.44 | 76:24 | CAT-2200 | Pertuzumab |
| 10950-11252 | 1.92 | 0.07 | 87:13 | 0.97 | 0.3 | 73:27 | CAT-2200 | Pertuzumab |
| 10950-11260 | 1.92 | 0.07 | 87:13 | 1.32 | NA | 79:21 | CAT-2200 | Pertuzumab |
| 10950-11264 | 1.92 | 0.07 | 87:13 | 1.47 | 0.18 | 81:19 | CAT-2200 | Pertuzumab |
| 10952-11398 | 1.08 | 1.48 | 75:25 | 2.33 | NA | 91:9 | CAT-2200 | Pertuzumab |
| 10953-11411 | 1.03 | 1.32 | 74:26 | 2.3 | 0.11 | 91:9 | CAT-2200 | Pertuzumab |
| 10962-11296 | 0.65 | 0.18 | 66:34 | 0.76 | NA | 68:32 | CAT-2200 | Pertuzumab |
| 10962-11350 | 0.65 | 0.18 | 66:34 | 0.22 | NA | 55:45 | CAT-2200 | Pertuzumab |
| 10965-11293 | 0.39 | 0.1 | 60:40 | 0.92 | NA | 72:28 | CAT-2200 | Pertuzumab |
| 10965-11347 | 0.39 | 0.1 | 60:40 | 0.77 | NA | 68:32 | CAT-2200 | Pertuzumab |
| 10966-11297 | 0.34 | 0.57 | 58:42 | 1.5 | NA | 82:18 | CAT-2200 | Pertuzumab |
| 10966-11351 | 0.34 | 0.57 | 58:42 | 1.05 | NA | 74:26 | CAT-2200 | Pertuzumab |
| 10969-11290 | 0.82 | 0.77 | 69:31 | 0.36 | NA | 59:41 | CAT-2200 | Pertuzumab |
| 10969-11344 | 0.82 | 0.77 | 69:31 | 0.22 | NA | 55:45 | CAT-2200 | Pertuzumab |
| 10970-11294 | 1.19 | 0.39 | 77:23 | 0.81 | NA | 69:31 | CAT-2200 | Pertuzumab |
| 10970-11348 | 1.19 | 0.39 | 77:23 | 0.41 | NA | 60:40 | CAT-2200 | Pertuzumab |
| 10973-11291 | 1.3 | 0.16 | 79:21 | 1.18 | NA | 76:24 | CAT-2200 | Pertuzumab |
| 10973-11345 | 1.3 | 0.16 | 79:21 | 0.82 | NA | 69:31 | CAT-2200 | Pertuzumab |
| 10974-11295 | 1.39 | 0.29 | 80:20 | 1.6 | NA | 83:17 | CAT-2200 | Pertuzumab |
| 10974-11349 | 1.39 | 0.29 | 80:20 | 1.27 | 0.16 | 78:22 | CAT-2200 | Pertuzumab |
| 10977-11292 | 0.59 | 0.16 | 64:36 | 0.26 | NA | 56:44 | CAT-2200 | Pertuzumab |
| 10977-11346 | 0.59 | 0.16 | 64:36 | 0.02 | NA | 50:50 | CAT-2200 | Pertuzumab |
| 10978-11296 | 0.76 | 0.24 | 68:32 | 0.76 | NA | 68:32 | CAT-2200 | Pertuzumab |
| 10978-11350 | 0.76 | 0.24 | 68:32 | 0.22 | NA | 55:45 | CAT-2200 | Pertuzumab |
| 10981-11293 | 0.41 | 0.06 | 60:40 | 0.92 | NA | 72:28 | CAT-2200 | Pertuzumab |
| 10981-11347 | 0.41 | 0.06 | 60:40 | 0.77 | NA | 68:32 | CAT-2200 | Pertuzumab |
| 10982-11297 | 0.58 | 0.1 | 64:36 | 1.5 | NA | 82:18 | CAT-2200 | Pertuzumab |
| 10982-11351 | 0.58 | 0.1 | 64:36 | 1.05 | NA | 74:26 | CAT-2200 | Pertuzumab |
| 10983-11306 | 1.21 | 0.36 | 77:23 | 2.48 | 0.22 | 92:8 | CAT-2200 | Pertuzumab |
| 10984-11376 | 0.65 | 0.2 | 66:34 | 1.5 | 0.39 | 82:18 | CAT-2200 | Pertuzumab |
| 10985-11381 | −0.04 | 0.01 | 49:51 | 1.7 | NA | 85:15 | CAT-2200 | Pertuzumab |
| 10986-11391 | 0.46 | 0.44 | 61:39 | 2.71 | 0.33 | 94:6 | CAT-2200 | Pertuzumab |
| 10987-11249 | 3.04 | 1.13 | 95:5 | 0.77 | 0.24 | 68:32 | CAT-2200 | Pertuzumab |
| 10987-11257 | 3.04 | 1.13 | 95:5 | 1.12 | 0.28 | 75:25 | CAT-2200 | Pertuzumab |
| 10987-11261 | 3.04 | 1.13 | 95:5 | 1.04 | 0.26 | 74:26 | CAT-2200 | Pertuzumab |
| 10989-11251 | 2.73 | 0.64 | 94:6 | 0.95 | NA | 72:28 | CAT-2200 | Pertuzumab |
| 10989-11259 | 2.73 | 0.64 | 94:6 | 1.47 | NA | 81:19 | CAT-2200 | Pertuzumab |
| 10989-11263 | 2.73 | 0.64 | 94:6 | 1.5 | 0.7 | 82:18 | CAT-2200 | Pertuzumab |
| 10992-11410 | 1.64 | 0.16 | 84:16 | 2.15 | NA | 90:10 | CAT-2200 | Pertuzumab |
| 10998-11307 | 1.24 | 0.55 | 78:22 | 2.3 | 0.17 | 91:9 | CAT-2200 | Pertuzumab |
| 10998-11314 | 1.24 | 0.55 | 78:22 | 3.06 | 0.45 | 96:4 | CAT-2200 | Pertuzumab |
| 10999-11377 | 0.8 | 0.41 | 69:31 | 2.22 | 0.65 | 90:10 | CAT-2200 | Pertuzumab |
| 11000-11382 | 0.28 | 0.03 | 57:43 | 1.67 | NA | 84:16 | CAT-2200 | Pertuzumab |
| 11001-11392 | 0.77 | 0.13 | 68:32 | 2.75 | 0.27 | 94:6 | CAT-2200 | Pertuzumab |
| 11002-11250 | 2.87 | 1.15 | 95:5 | 0.69 | NA | 67:33 | CAT-2200 | Pertuzumab |
| 11002-11258 | 2.87 | 1.15 | 95:5 | 1.22 | NA | 77:23 | CAT-2200 | Pertuzumab |
| 11002-11262 | 2.87 | 1.15 | 95:5 | 1.18 | 0.44 | 76:24 | CAT-2200 | Pertuzumab |
| 11004-11252 | 2.38 | 0.52 | 92:8 | 0.97 | 0.3 | 73:27 | CAT-2200 | Pertuzumab |
| 11004-11260 | 2.38 | 0.52 | 92:8 | 1.32 | NA | 79:21 | CAT-2200 | Pertuzumab |
| 11004-11264 | 2.38 | 0.52 | 92:8 | 1.47 | 0.18 | 81:19 | CAT-2200 | Pertuzumab |
| 11006-11398 | 1.7 | 0.54 | 85:15 | 2.33 | NA | 91:9 | CAT-2200 | Pertuzumab |
| 11007-11411 | 1.65 | 0.61 | 84:16 | 2.3 | 0.11 | 91:9 | CAT-2200 | Pertuzumab |
| 11025-11284 | 1.38 | 0.88 | 80:20 | 3.02 | 0.11 | 95:5 | CAT-2200 | Pertuzumab |
| 11026-11270 | 1.8 | 1.64 | 86:14 | 2.51 | 0.42 | 92:8 | CAT-2200 | Pertuzumab |
| 11027-11281 | 1.13 | 0.17 | 76:24 | 1.42 | 1.88 | 81:19 | CAT-2200 | Pertuzumab |
| 11027-11287 | 1.13 | 0.17 | 76:24 | 1.71 | 1.6 | 85:15 | CAT-2200 | Pertuzumab |
| 11028-11275 | 1.55 | 0.72 | 82:18 | 3.15 | 0.91 | 96:4 | CAT-2200 | Pertuzumab |
| 11029-11282 | 1.21 | NA | 77:23 | 2.8 | NA | 94:6 | CAT-2200 | Pertuzumab |
| 11029-11288 | 1.21 | NA | 77:23 | 2.83 | NA | 94:6 | CAT-2200 | Pertuzumab |
| 11030-11276 | 1.38 | 0.39 | 80:20 | 2.63 | 0.26 | 93:7 | CAT-2200 | Pertuzumab |
| 11030-11285 | 1.38 | 0.39 | 80:20 | 3 | 0.73 | 95:5 | CAT-2200 | Pertuzumab |
| 11032-11199 | 1.72 | 0.2 | 85:15 | 2.38 | 0.61 | 92:8 | CAT-2200 | Pertuzumab |
| 11033-11209 | 1.72 | 1.34 | 85:15 | 1.69 | NA | 84:16 | CAT-2200 | Pertuzumab |
| 11033-11215 | 1.72 | 1.34 | 85:15 | 2.31 | 0.42 | 91:9 | CAT-2200 | Pertuzumab |
| 11034-11204 | 1.43 | 0.25 | 81:19 | 3.12 | 0.61 | 96:4 | CAT-2200 | Pertuzumab |
| 11035-11212 | 1.81 | 0.46 | 86:14 | 2.04 | 0.12 | 88:12 | CAT-2200 | Pertuzumab |
| 11035-11218 | 1.81 | 0.46 | 86:14 | 3.12 | 0.48 | 96:4 | CAT-2200 | Pertuzumab |
| 11036-11221 | 1.35 | 0.18 | 79:21 | 1.38 | 0.52 | 80:20 | CAT-2200 | Pertuzumab |
| 11039-11224 | 0.9 | 0.46 | 71:29 | 0.62 | 0.2 | 65:35 | CAT-2200 | Pertuzumab |
| 11042-11273 | 0.83 | 1.03 | 70:30 | 1.75 | 0.25 | 85:15 | CAT-2200 | Pertuzumab |
| 11043-11181 | 1.09 | 0.21 | 75:25 | 1.68 | 0.67 | 84:16 | CAT-2200 | Pertuzumab |
| 11044-11191 | 0.93 | 0.18 | 72:28 | 1.5 | 0.61 | 82:18 | CAT-2200 | Pertuzumab |
| 11055-11322 | −0.34 | 0.5 | 42:58 | 1.51 | 0.2 | 82:18 | CAT-2200 | Pertuzumab |
| 11056-11324 | 0.42 | NA | 60:40 | 0.84 | NA | 70:30 | CAT-2200 | Pertuzumab |
| 11061-11273 | 1.23 | 0.45 | 77:23 | 1.75 | 0.25 | 85:15 | CAT-2200 | Pertuzumab |
| 11062-11323 | 0.54 | NA | 63:37 | 0.15 | NA | 54:46 | CAT-2200 | Pertuzumab |
| 11063-11325 | 0.83 | NA | 70:30 | −0.25 | NA | 44:56 | CAT-2200 | Pertuzumab |
| 11064-11201 | 1.64 | 0.68 | 84:16 | 2.18 | 0.61 | 90:10 | CAT-2200 | Pertuzumab |
| 11065-11206 | 1.84 | 0.08 | 86:14 | 2.72 | 0.33 | 94:6 | CAT-2200 | Pertuzumab |
| 11066-11181 | 0.91 | 0.15 | 71:29 | 1.68 | 0.67 | 84:16 | CAT-2200 | Pertuzumab |
| 11076-11355 | −0.4 | NA | 40:60 | 0.7 | NA | 67:33 | CAT-2200 | Pertuzumab |
| 11087-11179 | 1.98 | 0.49 | 88:12 | 1.83 | NA | 86:14 | CAT-2200 | Pertuzumab |
| 11088-11185 | 1.63 | 0.48 | 84:16 | 2.29 | 0.29 | 91:9 | CAT-2200 | Pertuzumab |
| 11089-11189 | 1.77 | 0.2 | 85:15 | 1.64 | NA | 84:16 | CAT-2200 | Pertuzumab |
| 11096-11272 | 1.75 | 0.63 | 85:15 | 2.62 | 0.05 | 93:7 | CAT-2200 | Pertuzumab |
| 11097-11277 | 1.87 | 0.16 | 87:13 | 2.22 | NA | 90:10 | CAT-2200 | Pertuzumab |
| 11098-11200 | 2.65 | 0.47 | 93:7 | 2.21 | 0.1 | 90:10 | CAT-2200 | Pertuzumab |
| 11099-11210 | 2.31 | 0.64 | 91:9 | 0.9 | NA | 71:29 | CAT-2200 | Pertuzumab |
| 11099-11216 | 2.31 | 0.64 | 91:9 | 1.77 | 0.35 | 85:15 | CAT-2200 | Pertuzumab |
| 11100-11205 | 2.58 | 0.19 | 93:7 | 2.51 | 0.72 | 92:8 | CAT-2200 | Pertuzumab |
| 11101-11213 | 2.44 | 0.37 | 92:8 | 1.24 | NA | 78:22 | CAT-2200 | Pertuzumab |
| 11101-11219 | 2.44 | 0.37 | 92:8 | 2.13 | 0.34 | 89:11 | CAT-2200 | Pertuzumab |
| 11102-11180 | 2.21 | 0.22 | 90:10 | 1.95 | 0.23 | 88:12 | CAT-2200 | Pertuzumab |
| 11103-11186 | 2.04 | 0.32 | 88:12 | 1.52 | 0.1 | 82:18 | CAT-2200 | Pertuzumab |
| 11104-11190 | 2.37 | 0.11 | 91:9 | 1.9 | NA | 87:13 | CAT-2200 | Pertuzumab |
| 11105-11195 | 1.93 | NA | 87:13 | −0.15 | 0.29 | 46:54 | CAT-2200 | Pertuzumab |
| 11106-11225 | 1.83 | NA | 86:14 | 0.65 | NA | 66:34 | CAT-2200 | Pertuzumab |
| 11106-11229 | 1.83 | NA | 86:14 | 0.18 | 0.06 | 54:46 | CAT-2200 | Pertuzumab |
| 11107-11197 | 1.92 | 0.25 | 87:13 | 0.25 | 0.18 | 56:44 | CAT-2200 | Pertuzumab |
| 11108-11227 | 2.4 | NA | 92:8 | 0.94 | NA | 72:28 | CAT-2200 | Pertuzumab |
| 11108-11231 | 2.4 | NA | 92:8 | 0.15 | 0.33 | 54:46 | CAT-2200 | Pertuzumab |
| 11124-11202 | 2.1 | 0.67 | 89:11 | 2.94 | 1.29 | 95:5 | CAT-2200 | Pertuzumab |
| 11125-11207 | 2.13 | 0.27 | 89:11 | 2.85 | 1.01 | 95:5 | CAT-2200 | Pertuzumab |
| 11140-11202 | 2.29 | 0.32 | 91:9 | 2.94 | 1.29 | 95:5 | CAT-2200 | Pertuzumab |
| 11141-11207 | 2.1 | NA | 89:11 | 2.85 | 1.01 | 95:5 | CAT-2200 | Pertuzumab |
| 11142-11182 | 1.93 | 0.21 | 87:13 | 2.4 | 0.71 | 92:8 | CAT-2200 | Pertuzumab |
| 11143-11187 | 1.4 | 0.57 | 80:20 | 2.11 | 0.33 | 89:11 | CAT-2200 | Pertuzumab |
| 11144-11192 | 1.96 | 0.32 | 88:12 | 2.3 | 0.38 | 91:9 | CAT-2200 | Pertuzumab |
| 11148-11183 | 2.25 | 0.2 | 90:10 | 1.93 | 1.9 | 87:13 | CAT-2200 | Pertuzumab |
| 11149-11188 | 1.99 | 0.29 | 88:12 | 1.59 | 0.29 | 83:17 | CAT-2200 | Pertuzumab |
| 11150-11193 | 2.13 | 0.7 | 89:11 | 1.59 | 0.3 | 83:17 | CAT-2200 | Pertuzumab |
| 11156-11274 | 0.42 | 0.77 | 60:40 | 3.21 | 0.22 | 96:4 | CAT-2200 | Pertuzumab |
| 11157-11279 | 0.38 | 0.09 | 59:41 | 3.16 | 0.59 | 96:4 | CAT-2200 | Pertuzumab |
| 11158-11203 | 2.02 | NA | 88:12 | 2.41 | 0.13 | 92:8 | CAT-2200 | Pertuzumab |
| 11159-11211 | 2.02 | 0.05 | 88:12 | 0.82 | NA | 69:31 | CAT-2200 | Pertuzumab |
| 11159-11217 | 2.02 | 0.05 | 88:12 | 1.98 | 0.27 | 88:12 | CAT-2200 | Pertuzumab |
| 11160-11208 | 2.02 | NA | 88:12 | 2.42 | 0.22 | 92:8 | CAT-2200 | Pertuzumab |
| 11161-11214 | 2.08 | 0.26 | 89:11 | 1.55 | NA | 82:18 | CAT-2200 | Pertuzumab |
| 11161-11220 | 2.08 | 0.26 | 89:11 | 2.4 | 0.71 | 92:8 | CAT-2200 | Pertuzumab |
| 11162-11184 | 1.13 | 0.65 | 76:24 | 1.09 | NA | 75:25 | CAT-2200 | Pertuzumab |
| 11163-11194 | 1.16 | 0.09 | 76:24 | 1.75 | NA | 85:15 | CAT-2200 | Pertuzumab |
| 11164-11196 | 0.93 | 0.3 | 72:28 | 1.69 | 0.58 | 84:16 | CAT-2200 | Pertuzumab |
| 11165-11226 | 0.92 | NA | 72:28 | 1.03 | NA | 74:26 | CAT-2200 | Pertuzumab |
| 11165-11230 | 0.92 | NA | 72:28 | 1.71 | 0.18 | 85:15 | CAT-2200 | Pertuzumab |
| 11166-11198 | 0.66 | 0.17 | 66:34 | 2.32 | 0.85 | 91:9 | CAT-2200 | Pertuzumab |
| 11167-11228 | 0.85 | NA | 70:30 | 1.19 | NA | 77:23 | CAT-2200 | Pertuzumab |
| 11167-11232 | 0.85 | NA | 70:30 | 1.84 | 0.11 | 86:14 | CAT-2200 | Pertuzumab |

TABLE 5B
LCCA results for K-K-derived K-L designs that passed the performance cut-off criteria

| | Column 2 | Column 3 | | Column 5 | Column 6 | | | |
|---|---|---|---|---|---|---|---|---|
| | H1L1:H1L2 | H1L1:H1L2 range | Column 4 | H2L2:H2L1 | H2L2:H2L1 range | Column 7 | | |
| | normalized | of normalized | H1L1:H1L2 | normalized | of normalized | H2L2:H2L1 | | |
| Column 1 | median scalar | scalar value | normalized | median scalar | scalar value | normalized | Column 8 | Column 9 |
| Unique identifier | value In(r1/f1) | In(r1/f1) | median ratio | value In(r1/f1) | In(r1/f1) | median ratio | H1L1 Fab | H2L2 Fab |
| 10621-10731 | 0.49 | 0.43 | 62:38 | 0.37 | 0.09 | 59:41 | CAT-2200 | Pertuzumab |
| 10621-10733 | 0.49 | 0.43 | 62:38 | 0.72 | 3.76 | 67:33 | CAT-2200 | Pertuzumab |
| 10622-10736 | 0.69 | NA | 67:33 | −0.11 | NA | 47:53 | CAT-2200 | Pertuzumab |
| 10622-10742 | 0.69 | NA | 67:33 | −0.11 | NA | 47:53 | CAT-2200 | Pertuzumab |
| 10623-10739 | 1.06 | NA | 74:26 | −0.03 | NA | 49:51 | CAT-2200 | Pertuzumab |
| 10623-10745 | 1.06 | NA | 74:26 | −0.08 | NA | 48:52 | CAT-2200 | Pertuzumab |
| 10624-10700 | −0.25 | NA | 44:56 | 0.57 | NA | 64:36 | CAT-2200 | Pertuzumab |
| 10625-10701 | 0.59 | 0.07 | 64:36 | −0.04 | 0.18 | 49:51 | CAT-2200 | Pertuzumab |
| 10629-10695 | 0.76 | NA | 68:32 | −0.36 | NA | 41:59 | CAT-2200 | Pertuzumab |
| 10631-10722 | 1.1 | 0.23 | 75:25 | 0.87 | 1.04 | 70:30 | CAT-2200 | Pertuzumab |
| 10632-10724 | 2.16 | 0.67 | 90:10 | 0.61 | 0.27 | 65:35 | CAT-2200 | Pertuzumab |
| 10640-10713 | 0.08 | 0.48 | 52:48 | 1.51 | 0.81 | 82:18 | CAT-2200 | Pertuzumab |
| 10652-10702 | −0.11 | 0.41 | 47:53 | 3.29 | 1.58 | 96:4 | CAT-2200 | Pertuzumab |
| 10652-10734 | −0.11 | 0.41 | 47:53 | 2.67 | 0.39 | 94:6 | CAT-2200 | Pertuzumab |
| 10652-10755 | −0.11 | 0.41 | 47:53 | 1.42 | NA | 81:19 | CAT-2200 | Pertuzumab |
| 10656-10758 | −0.13 | 0.61 | 47:53 | 2.54 | 0.65 | 93:7 | CAT-2200 | Pertuzumab |
| 10657-10760 | 0.53 | 0.46 | 63:37 | 2.85 | 1.28 | 95:5 | CAT-2200 | Pertuzumab |
| 10658-10720 | 0.07 | NA | 52:48 | 0.83 | 0.66 | 70:30 | CAT-2200 | Pertuzumab |
| 10659-10720 | 0.28 | 0.12 | 57:43 | 0.83 | 0.66 | 70:30 | CAT-2200 | Pertuzumab |
| 10662-10731 | 0.63 | 0.31 | 65:35 | 0.37 | 0.09 | 59:41 | CAT-2200 | Pertuzumab |
| 10662-10733 | 0.63 | 0.31 | 65:35 | 0.72 | 3.76 | 67:33 | CAT-2200 | Pertuzumab |
| 10664-10722 | 0.94 | 0.05 | 72:28 | 0.87 | 1.04 | 70:30 | CAT-2200 | Pertuzumab |
| 10665-10724 | 2.32 | 0.27 | 91:9 | 0.61 | 0.27 | 65:35 | CAT-2200 | Pertuzumab |
| 10671-10751 | 0.56 | NA | 64:36 | 0.15 | 0.32 | 54:46 | CAT-2200 | Pertuzumab |
| 10674-10749 | 1.13 | 0.18 | 76:24 | 0.18 | 0.21 | 54:46 | CAT-2200 | Pertuzumab |
| 10676-10750 | 0.81 | 0.23 | 69:31 | 0.23 | 0.19 | 56:44 | CAT-2200 | Pertuzumab |
| 10677-10751 | 0.78 | 0.39 | 69:31 | 0.15 | 0.32 | 54:46 | CAT-2200 | Pertuzumab |
| 10678-10737 | 0.91 | 0.37 | 71:29 | 1.09 | 0.39 | 75:25 | CAT-2200 | Pertuzumab |
| 10678-10743 | 0.91 | 0.37 | 71:29 | 1.09 | 0.91 | 75:25 | CAT-2200 | Pertuzumab |
| 10679-10738 | 0.61 | 0.08 | 65:35 | 1.35 | NA | 79:21 | CAT-2200 | Pertuzumab |
| 10679-10744 | 0.61 | 0.08 | 65:35 | 1.29 | 0.69 | 78:22 | CAT-2200 | Pertuzumab |
| 10680-10740 | 1.23 | 0.47 | 77:23 | 1.19 | 0.37 | 77:23 | CAT-2200 | Pertuzumab |
| 10680-10746 | 1.23 | 0.47 | 77:23 | 1.02 | 0.38 | 73:27 | CAT-2200 | Pertuzumab |
| 10681-10741 | 0.92 | 0.26 | 72:28 | 2.47 | 1.3 | 92:8 | CAT-2200 | Pertuzumab |
| 10681-10747 | 0.92 | 0.26 | 72:28 | 1.19 | 0.73 | 77:23 | CAT-2200 | Pertuzumab |
| 10682-10718 | 2.12 | 0.77 | 89:11 | 0.55 | 0.61 | 63:37 | CAT-2200 | Pertuzumab |
| 10683-10720 | 0.82 | 0.33 | 69:31 | 0.83 | 0.66 | 70:30 | CAT-2200 | Pertuzumab |
| 10684-10706 | 1.94 | 0.25 | 87:13 | 0.53 | 0.26 | 63:37 | CAT-2200 | Pertuzumab |
| 10685-10719 | 2.07 | 0.27 | 89:11 | 1.01 | 0.14 | 73:27 | CAT-2200 | Pertuzumab |
| 10685-10726 | 2.07 | 0.27 | 89:11 | 1.46 | 0.39 | 81:19 | CAT-2200 | Pertuzumab |
| 10687-10722 | 1.04 | 0.63 | 74:26 | 0.87 | 1.04 | 70:30 | CAT-2200 | Pertuzumab |
| 10688-10724 | 2.24 | 0.66 | 90:10 | 0.61 | 0.27 | 65:35 | CAT-2200 | Pertuzumab |
| 10689-10707 | 1.13 | 0.38 | 76:24 | 0.53 | 0.74 | 63:37 | CAT-2200 | Pertuzumab |
| 10690-10727 | 2.1 | 0.46 | 89:11 | 1.42 | 0.09 | 81:19 | CAT-2200 | Pertuzumab |
| 10621-10731 | 0.49 | 0.43 | 62:38 | 0.37 | 0.09 | 59:41 | CAT-2200 | Pertuzumab |

TABLE 6A
Antigen binding and thermal stability assessment of the K-L designs that passed the LCCA performance cut-off criteria
Table 6A-K-L designs

| | | | | | | Column 7 | | Column 9 | | Column 11 | | Column 13 | | |
|---|---|---|---|---|---|---|---|---|---|---|---|---|---|---|
| | | Column 3 | | Column 5 | | Change in | | Change in | | Change in | | Change in | | |
| | | Change in | | Change in | Column 6 | DFS values | Column 8 | DFS values | Column 10 | DFS values | Column 12 | DFS values | | |
| Column 1 | Column 2 | KD of H1L1 | Column 4 | KD of H2L2 | DFS values | of H1L1 | DFS values | of H2L2 | DFS values | KD of H1L1 | DFS values | of H2L2 | | |
| Unique | KD of H1L1 | Fab vs wild | KD of H2L2 | Fab vs wild | of H1L1 | Fab vs wild | of H2L2 | Fab vs wild | of H1L1 | Fab vs wild | of H2L2 | Fab vs wild | Column 14 | Column 15 |
| identifier | Fab (nM) | type | Fab (nM) | type | Fab (° C.) | type | Fab (° C.) | type | Fab (° C.) | type | Fab (° C.) | type | H1L1 Fab | H2L2 Fab |
| 10771-11335 | 0.27 | 0.00 | ND | ND | 77.0 | 0.0 | ND | ND | 70.7 | 0.0 | ND | ND | CAT-2200 | Pertuzumab |
| 10771-11360 | 0.27 | 0.00 | ND | ND | 77.0 | 0.0 | ND | ND | 70.7 | 0.0 | ND | ND | CAT-2200 | Pertuzumab |
| 10776-11312 | ND | ND | ND | ND | ND | ND | ND | ND | ND | ND | ND | ND | CAT-2200 | Pertuzumab |
| 10776-11321 | ND | ND | ND | ND | ND | ND | ND | ND | ND | ND | ND | ND | CAT-2200 | Pertuzumab |
| 10776-11353 | ND | ND | ND | ND | ND | ND | ND | ND | ND | ND | ND | ND | CAT-2200 | Pertuzumab |
| 10780-11417 | 0.22 | 0.09 | ND | ND | 76.5 | −0.5 | ND | ND | ND | ND | ND | ND | CAT-2200 | Pertuzumab |
| 10781-11300 | 0.22 | 0.09 | ND | ND | 76.5 | −0.5 | ND | ND | ND | ND | ND | ND | CAT-2200 | Pertuzumab |
| 10781-11419 | 0.22 | 0.09 | 7.76 | 0.02 | 76.5 | −0.5 | 80.5 | −1.5 | ND | ND | ND | ND | CAT-2200 | Pertuzumab |
| 10786-11299 | 0.23 | 0.08 | ND | ND | 75.7 | −1.3 | ND | ND | ND | ND | ND | ND | CAT-2200 | Pertuzumab |
| 10786-11418 | 0.23 | 0.08 | ND | ND | 75.7 | −1.3 | ND | ND | ND | ND | ND | ND | CAT-2200 | Pertuzumab |
| 10787-11301 | 0.23 | 0.08 | ND | ND | 75.7 | −1.3 | ND | ND | ND | ND | ND | ND | CAT-2200 | Pertuzumab |
| 10787-11420 | 0.23 | 0.08 | 7.76 | 0.02 | 75.7 | −1.3 | 80.5 | −1.5 | ND | ND | ND | ND | CAT-2200 | Pertuzumab |
| 10788-11422 | 0.23 | 0.08 | ND | ND | 75.7 | −1.3 | ND | ND | ND | ND | ND | ND | CAT-2200 | Pertuzumab |
| 10792-11305 | 0.23 | 0.08 | ND | ND | 75.7 | −1.3 | ND | ND | ND | ND | ND | ND | CAT-2200 | Pertuzumab |
| 10792-11424 | 0.23 | 0.08 | ND | ND | 75.7 | −1.3 | ND | ND | ND | ND | ND | ND | CAT-2200 | Pertuzumab |
| 10793-11375 | 0.23 | 0.08 | 8.13 | 0.00 | 75.7 | −1.3 | 80.5 | −1.5 | ND | ND | ND | ND | CAT-2200 | Pertuzumab |
| 10795-11390 | 0.34 | −0.10 | 7.92 | 0.01 | 74.0 | −3.0 | 80.7 | −1.3 | 69.7 | −1.0 | ND | ND | CAT-2200 | Pertuzumab |
| 10796-11396 | 0.34 | −0.10 | 8.15 | 0.00 | 74.0 | −3.0 | 82.5 | 0.5 | 69.7 | −1.0 | ND | ND | CAT-2200 | Pertuzumab |
| 10798-11256 | 0.34 | −0.10 | 7.78 | 0.02 | 74.0 | −3.0 | 81.5 | −0.5 | 69.7 | −1.0 | ND | ND | CAT-2200 | Pertuzumab |
| 10798-11268 | 0.34 | −0.10 | 8.25 | −0.01 | 74.0 | −3.0 | 81.5 | −0.5 | 69.7 | −1.0 | ND | ND | CAT-2200 | Pertuzumab |
| 10800-11416 | 0.34 | −0.10 | 8.26 | −0.01 | 74.0 | −3.0 | 78.0 | −4.0 | 69.7 | −1.0 | ND | ND | CAT-2200 | Pertuzumab |
| 10806-11326 | 0.27 | 0.00 | 7.65 | 0.02 | 69.7 | −7.3 | 83.2 | 1.3 | 65.5 | −5.2 | ND | ND | CAT-2200 | Pertuzumab |
| 10806-11369 | 0.27 | 0.00 | 6.98 | 0.06 | 69.7 | −7.3 | 81.7 | −0.3 | 65.5 | −5.2 | 78.4 | −0.3 | CAT-2200 | Pertuzumab |
| 10808-11308 | 0.27 | 0.00 | 7.41 | 0.04 | 69.7 | −7.3 | 81.0 | −1.0 | 65.5 | −5.2 | ND | ND | CAT-2200 | Pertuzumab |
| 10808-11315 | 0.27 | 0.00 | 7.91 | 0.01 | 69.7 | −7.3 | 80.3 | −1.7 | 65.5 | −5.2 | 77.1 | −1.6 | CAT-2200 | Pertuzumab |
| 10809-11378 | 0.27 | 0.00 | 8.46 | −0.02 | 69.7 | −7.3 | 80.2 | −1.8 | 65.5 | −5.2 | ND | ND | CAT-2200 | Pertuzumab |
| 10810-11383 | 0.27 | 0.00 | 8.31 | −0.01 | 69.7 | −7.3 | 78.8 | −3.2 | 65.5 | −5.2 | ND | ND | CAT-2200 | Pertuzumab |
| 10811-11386 | 0.27 | 0.00 | 7.92 | 0.01 | 69.7 | −7.3 | 80.7 | −1.3 | 65.5 | −5.2 | ND | ND | CAT-2200 | Pertuzumab |
| 10812-11393 | 0.27 | 0.00 | 8.15 | 0.00 | 69.7 | −7.3 | 82.5 | 0.5 | 65.5 | −5.2 | ND | ND | CAT-2200 | Pertuzumab |
| 10813-11311 | 0.27 | 0.00 | ND | ND | 69.7 | −7.3 | ND | ND | 65.5 | −5.2 | ND | ND | CAT-2200 | Pertuzumab |
| 10813-11320 | 0.27 | 0.00 | ND | ND | 69.7 | −7.3 | ND | ND | 65.5 | −5.2 | ND | ND | CAT-2200 | Pertuzumab |
| 10813-11352 | 0.27 | 0.00 | ND | ND | 69.7 | −7.3 | ND | ND | 65.5 | −5.2 | ND | ND | CAT-2200 | Pertuzumab |
| 10814-11233 | 0.27 | 0.00 | 8.28 | −0.01 | 69.7 | −7.3 | 80.8 | −1.1 | 65.5 | −5.2 | 78.2 | −0.5 | CAT-2200 | Pertuzumab |
| 10816-11253 | 0.27 | 0.00 | 7.78 | 0.02 | 69.7 | −7.3 | 81.5 | −0.5 | 65.5 | −5.2 | ND | ND | CAT-2200 | Pertuzumab |
| 10816-11265 | 0.27 | 0.00 | 8.25 | −0.01 | 69.7 | −7.3 | 81.5 | −0.5 | 65.5 | −5.2 | ND | ND | CAT-2200 | Pertuzumab |
| 10818-11404 | 0.27 | 0.00 | ND | ND | 69.7 | −7.3 | ND | ND | 65.5 | −5.2 | ND | ND | CAT-2200 | Pertuzumab |
| 10819-11407 | 0.27 | 0.00 | ND | ND | 69.7 | −7.3 | ND | ND | 65.5 | −5.2 | ND | ND | CAT-2200 | Pertuzumab |
| 10820-11413 | 0.27 | 0.00 | 8.26 | −0.01 | 69.7 | −7.3 | 78.0 | −4.0 | 65.5 | −5.2 | ND | ND | CAT-2200 | Pertuzumab |
| 10827-11373 | 0.25 | 0.04 | 7.57 | 0.03 | 60.0 | −17.0 | 82.2 | 0.3 | ND | ND | ND | ND | CAT-2200 | Pertuzumab |
| 10828-11327 | 0.25 | 0.04 | 7.65 | 0.02 | 60.0 | −17.0 | 83.2 | 1.3 | ND | ND | ND | ND | CAT-2200 | Pertuzumab |
| 10828-11370 | 0.25 | 0.04 | 6.98 | 0.06 | 60.0 | −17.0 | 81.7 | −0.3 | ND | ND | 78.4 | −0.3 | CAT-2200 | Pertuzumab |
| 10830-11309 | 0.25 | 0.04 | 7.41 | 0.04 | 60.0 | −17.0 | 81.0 | −1.0 | ND | ND | ND | ND | CAT-2200 | Pertuzumab |
| 10830-11316 | 0.25 | 0.04 | 7.91 | 0.01 | 60.0 | −17.0 | 80.3 | −1.7 | ND | ND | 77.1 | −1.6 | CAT-2200 | Pertuzumab |
| 10831-11379 | 0.25 | 0.04 | 8.46 | −0.02 | 60.0 | −17.0 | 80.2 | −1.8 | ND | ND | ND | ND | CAT-2200 | Pertuzumab |
| 10832-11384 | 0.25 | 0.04 | 8.31 | −0.01 | 60.0 | −17.0 | 78.8 | −3.2 | ND | ND | ND | ND | CAT-2200 | Pertuzumab |
| 10833-11387 | 0.25 | 0.04 | 7.92 | 0.01 | 60.0 | −17.0 | 80.7 | −1.3 | ND | ND | ND | ND | CAT-2200 | Pertuzumab |
| 10834-11394 | 0.25 | 0.04 | 8.15 | 0.00 | 60.0 | −17.0 | 82.5 | 0.5 | ND | ND | ND | ND | CAT-2200 | Pertuzumab |
| 10835-11234 | 0.25 | 0.04 | 8.28 | −0.01 | 60.0 | −17.0 | 80.8 | −1.1 | ND | ND | 78.2 | −0.5 | CAT-2200 | Pertuzumab |
| 10837-11254 | 0.25 | 0.04 | 7.78 | 0.02 | 60.0 | −17.0 | 81.5 | −0.5 | ND | ND | ND | ND | CAT-2200 | Pertuzumab |
| 10837-11266 | 0.25 | 0.04 | 8.25 | −0.01 | 60.0 | −17.0 | 81.5 | −0.5 | ND | ND | ND | ND | CAT-2200 | Pertuzumab |
| 10840-11408 | 0.25 | 0.04 | ND | ND | 60.0 | −17.0 | ND | ND | ND | ND | ND | ND | CAT-2200 | Pertuzumab |
| 10848-11328 | 0.27 | 0.01 | 7.65 | 0.02 | 67.7 | −9.3 | 83.2 | 1.3 | ND | ND | ND | ND | CAT-2200 | Pertuzumab |
| 10848-11371 | 0.27 | 0.01 | 6.98 | 0.06 | 67.7 | −9.3 | 81.7 | −0.3 | ND | ND | 78.4 | −0.3 | CAT-2200 | Pertuzumab |
| 10850-11310 | 0.27 | 0.01 | 7.41 | 0.04 | 67.7 | −9.3 | 81.0 | −1.0 | ND | ND | ND | ND | CAT-2200 | Pertuzumab |
| 10850-11317 | 0.27 | 0.01 | 7.91 | 0.01 | 67.7 | −9.3 | 80.3 | −1.7 | ND | ND | 77.1 | −1.6 | CAT-2200 | Pertuzumab |
| 10851-11380 | 0.27 | 0.01 | 8.46 | −0.02 | 67.7 | −9.3 | 80.2 | −1.8 | ND | ND | ND | ND | CAT-2200 | Pertuzumab |
| 10852-11385 | 0.27 | 0.01 | 8.31 | −0.01 | 67.7 | −9.3 | 78.8 | −3.2 | ND | ND | ND | ND | CAT-2200 | Pertuzumab |
| 10853-11388 | 0.27 | 0.01 | 7.92 | 0.01 | 67.7 | −9.3 | 80.7 | −1.3 | ND | ND | ND | ND | CAT-2200 | Pertuzumab |
| 10854-11395 | 0.27 | 0.01 | 8.15 | 0.00 | 67.7 | −9.3 | 82.5 | 0.5 | ND | ND | ND | ND | CAT-2200 | Pertuzumab |
| 10855-11312 | 0.27 | 0.01 | ND | ND | 67.7 | −9.3 | ND | ND | ND | ND | ND | ND | CAT-2200 | Pertuzumab |
| 10855-11321 | 0.27 | 0.01 | ND | ND | 67.7 | −9.3 | ND | ND | ND | ND | ND | ND | CAT-2200 | Pertuzumab |
| 10855-11353 | 0.27 | 0.01 | ND | ND | 67.7 | −9.3 | ND | ND | ND | ND | ND | ND | CAT-2200 | Pertuzumab |
| 10856-11235 | 0.27 | 0.01 | 8.28 | −0.01 | 67.7 | −9.3 | 80.8 | −1.1 | ND | ND | 78.2 | −0.5 | CAT-2200 | Pertuzumab |
| 10857-11243 | 0.27 | 0.01 | 10.65 | −0.12 | 67.7 | −9.3 | 78.8 | −3.2 | ND | ND | 75.5 | −3.2 | CAT-2200 | Pertuzumab |
| 10858-11255 | 0.27 | 0.01 | 7.78 | 0.02 | 67.7 | −9.3 | 81.5 | −0.5 | ND | ND | ND | ND | CAT-2200 | Pertuzumab |
| 10858-11267 | 0.27 | 0.01 | 8.25 | −0.01 | 67.7 | −9.3 | 81.5 | −0.5 | ND | ND | ND | ND | CAT-2200 | Pertuzumab |
| 10860-11406 | 0.27 | 0.01 | ND | ND | 67.7 | −9.3 | ND | ND | ND | ND | ND | ND | CAT-2200 | Pertuzumab |
| 10861-11409 | 0.27 | 0.01 | ND | ND | 67.7 | −9.3 | ND | ND | ND | ND | ND | ND | CAT-2200 | Pertuzumab |
| 10862-11414 | 0.27 | 0.01 | 8.26 | −0.01 | 67.7 | −9.3 | 78.0 | −4.0 | ND | ND | ND | ND | CAT-2200 | Pertuzumab |
| 10869-11390 | 0.21 | 0.11 | 7.92 | 0.01 | 73.7 | −3.3 | 80.7 | −1.3 | 68.9 | −1.8 | ND | ND | CAT-2200 | Pertuzumab |
| 10870-11396 | 0.21 | 0.11 | 8.15 | 0.00 | 73.7 | −3.3 | 82.5 | 0.5 | 68.9 | −1.8 | ND | ND | CAT-2200 | Pertuzumab |
| 10871-11240 | 0.21 | 0.11 | 8.28 | −0.01 | 73.7 | −3.3 | 80.8 | −1.1 | 68.9 | −1.8 | 78.2 | −0.5 | CAT-2200 | Pertuzumab |
| 10873-11256 | 0.21 | 0.11 | 7.78 | 0.02 | 73.7 | −3.3 | 81.5 | −0.5 | 68.9 | −1.8 | ND | ND | CAT-2200 | Pertuzumab |
| 10873-11268 | 0.21 | 0.11 | 8.25 | −0.01 | 73.7 | −3.3 | 81.5 | −0.5 | 68.9 | −1.8 | ND | ND | CAT-2200 | Pertuzumab |
| 10875-11416 | 0.21 | 0.11 | 8.26 | −0.01 | 73.7 | −3.3 | 78.0 | −4.0 | 68.9 | −1.8 | ND | ND | CAT-2200 | Pertuzumab |
| 10881-11412 | NB | −3.56 | 8.26 | −0.01 | ND | ND | 78.0 | −4.0 | ND | ND | ND | ND | CAT-2200 | Pertuzumab |
| 10883-11236 | NB | −3.56 | 8.28 | −0.01 | ND | ND | 80.8 | −1.1 | ND | ND | 78.2 | −0.5 | CAT-2200 | Pertuzumab |
| 10888-11415 | NB | −3.56 | 8.26 | −0.01 | ND | ND | 78.0 | −4.0 | ND | ND | ND | ND | CAT-2200 | Pertuzumab |
| 10898-11239 | ND | ND | 8.28 | −0.01 | ND | ND | 80.8 | −1.1 | ND | ND | 78.2 | −0.5 | CAT-2200 | Pertuzumab |
| 10923-11306 | 0.22 | 0.10 | 7.41 | 0.04 | 74.0 | −3.0 | 81.0 | −1.0 | ND | ND | ND | ND | CAT-2200 | Pertuzumab |
| 10924-11376 | 0.22 | 0.10 | 8.46 | −0.02 | 74.0 | −3.0 | 80.2 | −1.8 | ND | ND | ND | ND | CAT-2200 | Pertuzumab |
| 10926-11391 | 0.22 | 0.10 | 8.15 | 0.00 | 74.0 | −3.0 | 82.5 | 0.5 | ND | ND | ND | ND | CAT-2200 | Pertuzumab |
| 10927-11290 | 0.22 | 0.10 | ND | ND | 74.0 | −3.0 | ND | ND | ND | ND | ND | ND | CAT-2200 | Pertuzumab |
| 10927-11344 | 0.22 | 0.10 | 8.01 | 0.00 | 74.0 | −3.0 | 78.0 | −4.0 | ND | ND | ND | ND | CAT-2200 | Pertuzumab |
| 10928-11294 | 0.22 | 0.10 | 7.13 | 0.05 | 74.0 | −3.0 | 78.0 | −4.0 | ND | ND | 74.9 | −3.8 | CAT-2200 | Pertuzumab |
| 10928-11348 | 0.22 | 0.10 | ND | ND | 74.0 | −3.0 | ND | ND | ND | ND | ND | ND | CAT-2200 | Pertuzumab |
| 10929-11249 | 0.22 | 0.10 | 8.64 | −0.03 | 74.0 | −3.0 | 80.0 | −2.0 | ND | ND | 76.6 | −2.1 | CAT-2200 | Pertuzumab |
| 10929-11257 | 0.22 | 0.10 | 8.53 | −0.02 | 74.0 | −3.0 | 80.0 | −2.0 | ND | ND | 76.8 | −1.9 | CAT-2200 | Pertuzumab |
| 10929-11261 | 0.22 | 0.10 | ND | ND | 74.0 | −3.0 | ND | ND | ND | ND | ND | ND | CAT-2200 | Pertuzumab |
| 10931-11251 | 0.22 | 0.10 | 7.78 | 0.02 | 74.0 | −3.0 | 81.5 | −0.5 | ND | ND | ND | ND | CAT-2200 | Pertuzumab |
| 10931-11259 | 0.22 | 0.10 | ND | ND | 74.0 | −3.0 | 79.0 | −3.0 | ND | ND | ND | ND | CAT-2200 | Pertuzumab |
| 10931-11263 | 0.22 | 0.10 | 8.25 | −0.01 | 74.0 | −3.0 | 81.5 | −0.5 | ND | ND | ND | ND | CAT-2200 | Pertuzumab |
| 10934-11410 | 0.22 | 0.10 | 8.26 | −0.01 | 74.0 | −3.0 | 78.0 | −4.0 | ND | ND | ND | ND | CAT-2200 | Pertuzumab |
| 10942-11291 | 0.33 | −0.08 | ND | ND | 74.0 | −3.0 | ND | ND | ND | ND | ND | ND | CAT-2200 | Pertuzumab |
| 10942-11345 | 0.33 | −0.08 | 8.01 | 0.00 | 74.0 | −3.0 | 78.0 | −4.0 | ND | ND | ND | ND | CAT-2200 | Pertuzumab |
| 10943-11295 | 0.33 | −0.08 | 7.13 | 0.05 | 74.0 | −3.0 | 78.0 | −4.0 | ND | ND | 74.9 | −3.8 | CAT-2200 | Pertuzumab |
| 10943-11349 | 0.33 | −0.08 | ND | ND | 74.0 | −3.0 | ND | ND | ND | ND | ND | ND | CAT-2200 | Pertuzumab |
| 10944-11307 | 0.35 | −0.10 | 7.41 | 0.04 | 74.2 | −2.8 | 81.0 | −1.0 | ND | ND | ND | ND | CAT-2200 | Pertuzumab |
| 10944-11314 | 0.35 | −0.10 | 7.91 | 0.01 | 74.2 | −2.8 | 80.3 | −1.7 | ND | ND | 77.1 | −1.6 | CAT-2200 | Pertuzumab |
| 10945-11377 | 0.35 | −0.10 | 8.46 | −0.02 | 74.2 | −2.8 | 80.2 | −1.8 | ND | ND | ND | ND | CAT-2200 | Pertuzumab |
| 10946-11382 | 0.35 | −0.10 | 8.31 | −0.01 | 74.2 | −2.8 | 78.8 | −3.2 | ND | ND | ND | ND | CAT-2200 | Pertuzumab |
| 10947-11392 | 0.35 | −0.10 | 8.15 | 0.00 | 74.2 | −2.8 | 82.5 | 0.5 | ND | ND | ND | ND | CAT-2200 | Pertuzumab |
| 10948-11250 | 0.35 | −0.10 | 8.64 | −0.03 | 74.2 | −2.8 | 80.0 | −2.0 | ND | ND | 76.6 | −2.1 | CAT-2200 | Pertuzumab |
| 10948-11258 | 0.35 | −0.10 | 8.53 | −0.02 | 74.2 | −2.8 | 80.0 | −2.0 | ND | ND | 76.8 | −1.9 | CAT-2200 | Pertuzumab |
| 10948-11262 | 0.35 | −0.10 | ND | ND | 74.2 | −2.8 | ND | ND | ND | ND | ND | ND | CAT-2200 | Pertuzumab |
| 10950-11252 | 0.35 | −0.10 | 7.78 | 0.02 | 74.2 | −2.8 | 81.5 | −0.5 | ND | ND | ND | ND | CAT-2200 | Pertuzumab |
| 10950-11260 | 0.35 | −0.10 | ND | ND | 74.2 | −2.8 | 79.0 | −3.0 | ND | ND | ND | ND | CAT-2200 | Pertuzumab |
| 10950-11264 | 0.35 | −0.10 | 8.25 | −0.01 | 74.2 | −2.8 | 81.5 | −0.5 | ND | ND | ND | ND | CAT-2200 | Pertuzumab |
| 10952-11398 | 0.35 | −0.10 | 9.02 | −0.05 | 74.2 | −2.8 | 80.2 | −1.8 | ND | ND | 76.8 | −1.9 | CAT-2200 | Pertuzumab |
| 10953-11411 | 0.35 | −0.10 | 8.26 | −0.01 | 74.2 | −2.8 | 78.0 | −4.0 | ND | ND | ND | ND | CAT-2200 | Pertuzumab |
| 10962-11296 | ND | ND | 7.13 | 0.05 | ND | ND | 78.0 | −4.0 | ND | ND | 74.9 | −3.8 | CAT-2200 | Pertuzumab |
| 10962-11350 | ND | ND | ND | ND | ND | ND | ND | ND | ND | ND | ND | ND | CAT-2200 | Pertuzumab |
| 10965-11293 | ND | ND | ND | ND | ND | ND | ND | ND | ND | ND | ND | ND | CAT-2200 | Pertuzumab |
| 10965-11347 | ND | ND | 8.01 | 0.00 | ND | ND | 78.0 | −4.0 | ND | ND | ND | ND | CAT-2200 | Pertuzumab |
| 10966-11297 | ND | ND | 7.13 | 0.05 | ND | ND | 78.0 | −4.0 | ND | ND | 74.9 | −3.8 | CAT-2200 | Pertuzumab |
| 10966-11351 | ND | ND | ND | ND | ND | ND | ND | ND | ND | ND | ND | ND | CAT-2200 | Pertuzumab |
| 10969-11290 | ND | ND | ND | ND | ND | ND | ND | ND | ND | ND | ND | ND | CAT-2200 | Pertuzumab |
| 10969-11344 | ND | ND | 8.01 | 0.00 | ND | ND | 78.0 | −4.0 | ND | ND | ND | ND | CAT-2200 | Pertuzumab |
| 10970-11294 | ND | ND | 7.13 | 0.05 | ND | ND | 78.0 | −4.0 | ND | ND | 74.9 | −3.8 | CAT-2200 | Pertuzumab |
| 10970-11348 | ND | ND | ND | ND | ND | ND | ND | ND | ND | ND | ND | ND | CAT-2200 | Pertuzumab |
| 10973-11291 | 0.30 | −0.04 | ND | ND | 73.2 | −3.8 | ND | ND | 69.7 | −1.0 | ND | ND | CAT-2200 | Pertuzumab |
| 10973-11345 | 0.30 | −0.04 | 8.01 | 0.00 | 73.2 | −3.8 | 78.0 | −4.0 | 69.7 | −1.0 | ND | ND | CAT-2200 | Pertuzumab |
| 10974-11295 | 0.30 | −0.04 | 7.13 | 0.05 | 73.2 | −3.8 | 78.0 | −4.0 | 69.7 | −1.0 | 74.9 | −3.8 | CAT-2200 | Pertuzumab |
| 10974-11349 | 0.30 | −0.04 | ND | ND | 73.2 | −3.8 | ND | ND | 69.7 | −1.0 | ND | ND | CAT-2200 | Pertuzumab |
| 10977-11292 | ND | ND | ND | ND | ND | ND | ND | ND | ND | ND | ND | ND | CAT-2200 | Pertuzumab |
| 10977-11346 | ND | ND | 8.01 | 0.00 | ND | ND | 78.0 | −4.0 | ND | ND | ND | ND | CAT-2200 | Pertuzumab |
| 10978-11296 | ND | ND | 7.13 | 0.05 | ND | ND | 78.0 | −4.0 | ND | ND | 74.9 | −3.8 | CAT-2200 | Pertuzumab |
| 10978-11350 | ND | ND | ND | ND | ND | ND | ND | ND | ND | ND | ND | ND | CAT-2200 | Pertuzumab |
| 10981-11293 | ND | ND | ND | ND | ND | ND | ND | ND | ND | ND | ND | ND | CAT-2200 | Pertuzumab |
| 10981-11347 | ND | ND | 8.01 | 0.00 | ND | ND | 78.0 | −4.0 | ND | ND | ND | ND | CAT-2200 | Pertuzumab |
| 10982-11297 | ND | ND | 7.13 | 0.05 | ND | ND | 78.0 | −4.0 | ND | ND | 74.9 | −3.8 | CAT-2200 | Pertuzumab |
| 10982-11351 | ND | ND | ND | ND | ND | ND | ND | ND | ND | ND | ND | ND | CAT-2200 | Pertuzumab |
| 10983-11306 | 0.32 | −0.06 | 7.41 | 0.04 | 73.0 | −4.0 | 81.0 | −1.0 | 68.3 | −2.4 | ND | ND | CAT-2200 | Pertuzumab |
| 10984-11376 | 0.32 | −0.06 | 8.46 | −0.02 | 73.0 | −4.0 | 80.2 | −1.8 | 68.3 | −2.4 | ND | ND | CAT-2200 | Pertuzumab |
| 10985-11381 | 0.32 | −0.06 | 8.31 | −0.01 | 73.0 | −4.0 | 78.8 | −3.2 | 68.3 | −2.4 | ND | ND | CAT-2200 | Pertuzumab |
| 10986-11391 | 0.32 | −0.06 | 8.15 | 0.00 | 73.0 | −4.0 | 82.5 | 0.5 | 68.3 | −2.4 | ND | ND | CAT-2200 | Pertuzumab |
| 10987-11249 | 0.32 | −0.06 | 8.64 | −0.03 | 73.0 | −4.0 | 80.0 | −2.0 | 68.3 | −2.4 | 76.6 | −2.1 | CAT-2200 | Pertuzumab |
| 10987-11257 | 0.32 | −0.06 | 8.53 | −0.02 | 73.0 | −4.0 | 80.0 | −2.0 | 68.3 | −2.4 | 76.8 | −1.9 | CAT-2200 | Pertuzumab |
| 10987-11261 | 0.32 | −0.06 | ND | ND | 73.0 | −4.0 | ND | ND | 68.3 | −2.4 | ND | ND | CAT-2200 | Pertuzumab |
| 10989-11251 | 0.32 | −0.06 | 7.78 | 0.02 | 73.0 | −4.0 | 81.5 | −0.5 | 68.3 | −2.4 | ND | ND | CAT-2200 | Pertuzumab |
| 10989-11259 | 0.32 | −0.06 | ND | ND | 73.0 | −4.0 | 79.0 | −3.0 | 68.3 | −2.4 | ND | ND | CAT-2200 | Pertuzumab |
| 10989-11263 | 0.32 | −0.06 | 8.25 | −0.01 | 73.0 | −4.0 | 81.5 | −0.5 | 68.3 | −2.4 | ND | ND | CAT-2200 | Pertuzumab |
| 10992-11410 | 0.32 | −0.06 | 8.26 | −0.01 | 73.0 | −4.0 | 78.0 | −4.0 | 68.3 | −2.4 | ND | ND | CAT-2200 | Pertuzumab |
| 10998-11307 | 0.29 | −0.03 | 7.41 | 0.04 | 73.0 | −4.0 | 81.0 | −1.0 | 69.1 | −1.6 | ND | ND | CAT-2200 | Pertuzumab |
| 10998-11314 | 0.29 | −0.03 | 7.91 | 0.01 | 73.0 | −4.0 | 80.3 | −1.7 | 69.1 | −1.6 | 77.1 | −1.6 | CAT-2200 | Pertuzumab |
| 10999-11377 | 0.29 | −0.03 | 8.46 | −0.02 | 73.0 | −4.0 | 80.2 | −1.8 | 69.1 | −1.6 | ND | ND | CAT-2200 | Pertuzumab |
| 11000-11382 | 0.29 | −0.03 | 8.31 | −0.01 | 73.0 | −4.0 | 78.8 | −3.2 | 69.1 | −1.6 | ND | ND | CAT-2200 | Pertuzumab |
| 11001-11392 | 0.29 | −0.03 | 8.15 | 0.00 | 73.0 | −4.0 | 82.5 | 0.5 | 69.1 | −1.6 | ND | ND | CAT-2200 | Pertuzumab |
| 11002-11250 | 0.29 | −0.03 | 8.64 | −0.03 | 73.0 | −4.0 | 80.0 | −2.0 | 69.1 | −1.6 | 76.6 | −2.1 | CAT-2200 | Pertuzumab |
| 11002-11258 | 0.29 | −0.03 | 8.53 | −0.02 | 73.0 | −4.0 | 80.0 | −2.0 | 69.1 | −1.6 | 76.8 | −1.9 | CAT-2200 | Pertuzumab |
| 11002-11262 | 0.29 | −0.03 | ND | ND | 73.0 | −4.0 | ND | ND | 69.1 | −1.6 | ND | ND | CAT-2200 | Pertuzumab |
| 11004-11252 | 0.29 | −0.03 | 7.78 | 0.02 | 73.0 | −4.0 | 81.5 | −0.5 | 69.1 | −1.6 | ND | ND | CAT-2200 | Pertuzumab |
| 11004-11260 | 0.29 | −0.03 | ND | ND | 73.0 | −4.0 | 79.0 | −3.0 | 69.1 | −1.6 | ND | ND | CAT-2200 | Pertuzumab |
| 11004-11264 | 0.29 | −0.03 | 8.25 | −0.01 | 73.0 | −4.0 | 81.5 | −0.5 | 69.1 | −1.6 | ND | ND | CAT-2200 | Pertuzumab |
| 11006-11398 | 0.29 | −0.03 | 9.02 | −0.05 | 73.0 | −4.0 | 80.2 | −1.8 | 69.1 | −1.6 | 76.8 | −1.9 | CAT-2200 | Pertuzumab |
| 11007-11411 | 0.29 | −0.03 | 8.26 | −0.01 | 73.0 | −4.0 | 78.0 | −4.0 | 69.1 | −1.6 | ND | ND | CAT-2200 | Pertuzumab |
| 11025-11284 | 0.26 | 0.03 | 7.38 | 0.04 | 73.8 | −3.2 | 81.2 | −0.8 | ND | ND | 77.6 | −1.1 | CAT-2200 | Pertuzumab |
| 11026-11270 | 0.26 | 0.03 | 7.30 | 0.04 | 73.8 | −3.2 | 79.5 | −2.5 | ND | ND | 76.0 | −2.7 | CAT-2200 | Pertuzumab |
| 11027-11281 | 0.26 | 0.03 | ND | ND | 73.8 | −3.2 | ND | ND | ND | ND | ND | ND | CAT-2200 | Pertuzumab |
| 11027-11287 | 0.26 | 0.03 | 5.81 | 0.14 | 73.8 | −3.2 | 81.8 | −0.2 | ND | ND | ND | ND | CAT-2200 | Pertuzumab |
| 11028-11275 | 0.26 | 0.03 | 7.57 | 0.03 | 73.8 | −3.2 | 80.8 | −1.2 | ND | ND | 77.4 | −1.3 | CAT-2200 | Pertuzumab |
| 11029-11282 | 0.26 | 0.03 | ND | ND | 73.8 | −3.2 | ND | ND | ND | ND | ND | ND | CAT-2200 | Pertuzumab |
| 11029-11288 | 0.26 | 0.03 | 7.71 | 0.02 | 73.8 | −3.2 | 81.0 | −1.0 | ND | ND | 77.5 | −1.2 | CAT-2200 | Pertuzumab |
| 11030-11276 | 0.26 | 0.03 | 7.00 | 0.06 | 73.8 | −3.2 | 81.3 | −0.7 | ND | ND | 77.9 | −0.8 | CAT-2200 | Pertuzumab |
| 11030-11285 | 0.26 | 0.03 | 7.44 | 0.03 | 73.8 | −3.2 | 81.5 | −0.5 | ND | ND | ND | ND | CAT-2200 | Pertuzumab |
| 11032-11199 | 0.26 | 0.03 | 8.09 | 0.00 | 73.8 | −3.2 | 80.0 | −2.0 | ND | ND | 76.5 | −2.2 | CAT-2200 | Pertuzumab |
| 11033-11209 | 0.26 | 0.03 | ND | ND | 73.8 | −3.2 | ND | ND | ND | ND | ND | ND | CAT-2200 | Pertuzumab |
| 11033-11215 | 0.26 | 0.03 | 7.98 | 0.00 | 73.8 | −3.2 | 75.5 | −6.5 | ND | ND | ND | ND | CAT-2200 | Pertuzumab |
| 11034-11204 | 0.26 | 0.03 | 8.11 | 0.00 | 73.8 | −3.2 | 80.8 | −1.2 | ND | ND | 77.5 | −1.2 | CAT-2200 | Pertuzumab |
| 11035-11212 | 0.26 | 0.03 | ND | ND | 73.8 | −3.2 | ND | ND | ND | ND | ND | ND | CAT-2200 | Pertuzumab |
| 11035-11218 | 0.26 | 0.03 | 8.06 | 0.00 | 73.8 | −3.2 | 77.0 | −5.0 | ND | ND | 74.3 | −4.4 | CAT-2200 | Pertuzumab |
| 11036-11221 | 0.26 | 0.03 | 7.72 | 0.02 | 73.8 | −3.2 | 75.5 | −6.5 | ND | ND | ND | ND | CAT-2200 | Pertuzumab |
| 11039-11224 | 0.26 | 0.03 | ND | ND | 73.8 | −3.2 | ND | ND | ND | ND | ND | ND | CAT-2200 | Pertuzumab |
| 11042-11273 | 0.18 | 0.19 | 7.19 | 0.05 | 72.7 | −4.3 | 81.2 | −0.8 | ND | ND | ND | ND | CAT-2200 | Pertuzumab |
| 11043-11181 | 0.18 | 0.19 | 7.30 | 0.04 | 72.7 | −4.3 | 75.8 | −6.2 | ND | ND | ND | ND | CAT-2200 | Pertuzumab |
| 11044-11191 | 0.18 | 0.19 | ND | ND | 72.7 | −4.3 | 76.0 | −6.0 | ND | ND | ND | ND | CAT-2200 | Pertuzumab |
| 11055-11322 | 0.12 | 0.02 | ND | ND | 71.8 | −4.5 | ND | ND | ND | ND | ND | ND | CAT-2200 | Pertuzumab |
| 11056-11324 | 0.12 | 0.02 | ND | ND | 71.8 | −4.5 | ND | ND | ND | ND | ND | ND | CAT-2200 | Pertuzumab |
| 11061-11273 | 0.28 | −0.02 | 7.19 | 0.05 | 72.3 | −4.7 | 81.2 | −0.8 | 68.6 | −2.1 | ND | ND | CAT-2200 | Pertuzumab |
| 11062-11323 | 0.28 | −0.02 | ND | ND | 72.3 | −4.7 | ND | ND | 68.6 | −2.1 | ND | ND | CAT-2200 | Pertuzumab |
| 11063-11325 | 0.28 | −0.02 | ND | ND | 72.3 | −4.7 | ND | ND | 68.6 | −2.1 | ND | ND | CAT-2200 | Pertuzumab |
| 11064-11201 | 0.28 | −0.02 | 8.09 | 0.00 | 72.3 | −4.7 | 80.0 | −2.0 | 68.6 | −2.1 | 76.5 | −2.2 | CAT-2200 | Pertuzumab |
| 11065-11206 | 0.28 | −0.02 | 8.11 | 0.00 | 72.3 | −4.7 | 80.8 | −1.2 | 68.6 | −2.1 | 77.5 | −1.2 | CAT-2200 | Pertuzumab |
| 11066-11181 | 0.28 | −0.02 | 7.30 | 0.04 | 72.3 | −4.7 | 75.8 | −6.2 | 68.6 | −2.1 | ND | ND | CAT-2200 | Pertuzumab |
| 11076-11355 | ND | ND | ND | ND | ND | ND | ND | ND | ND | ND | ND | ND | CAT-2200 | Pertuzumab |
| 11087-11179 | 0.37 | −0.13 | 7.30 | 0.04 | 72.2 | −4.8 | 75.8 | −6.2 | ND | ND | ND | ND | CAT-2200 | Pertuzumab |
| 11088-11185 | 0.37 | −0.13 | 7.40 | 0.04 | 72.2 | −4.8 | 67.7 | −14.3 | ND | ND | ND | ND | CAT-2200 | Pertuzumab |
| 11089-11189 | 0.37 | −0.13 | ND | ND | 72.2 | −4.8 | 76.0 | −6.0 | ND | ND | ND | ND | CAT-2200 | Pertuzumab |
| 11096-11272 | 0.36 | −0.12 | 7.19 | 0.05 | 72.7 | −4.3 | 81.2 | −0.8 | 68.8 | −1.9 | ND | ND | CAT-2200 | Pertuzumab |
| 11097-11277 | 0.36 | −0.12 | 7.00 | 0.06 | 72.7 | −4.3 | 81.3 | −0.7 | 68.8 | −1.9 | 77.9 | −0.8 | CAT-2200 | Pertuzumab |
| 11098-11200 | 0.36 | −0.12 | 8.09 | 0.00 | 72.7 | −4.3 | 80.0 | −2.0 | 68.8 | −1.9 | 76.5 | −2.2 | CAT-2200 | Pertuzumab |
| 11099-11210 | 0.36 | −0.12 | ND | ND | 72.7 | −4.3 | ND | ND | 68.8 | −1.9 | ND | ND | CAT-2200 | Pertuzumab |
| 11099-11216 | 0.36 | −0.12 | 7.98 | 0.00 | 72.7 | −4.3 | 75.5 | −6.5 | 68.8 | −1.9 | ND | ND | CAT-2200 | Pertuzumab |
| 11100-11205 | 0.36 | −0.12 | 8.11 | 0.00 | 72.7 | −4.3 | 80.8 | −1.2 | 68.8 | −1.9 | 77.5 | −1.2 | CAT-2200 | Pertuzumab |
| 11101-11213 | 0.36 | −0.12 | ND | ND | 72.7 | −4.3 | ND | ND | 68.8 | −1.9 | ND | ND | CAT-2200 | Pertuzumab |
| 11101-11219 | 0.36 | −0.12 | 8.06 | 0.00 | 72.7 | −4.3 | 77.0 | −5.0 | 68.8 | −1.9 | 74.3 | −4.4 | CAT-2200 | Pertuzumab |
| 11102-11180 | 0.36 | −0.12 | 7.30 | 0.04 | 72.7 | −4.3 | 75.8 | −6.2 | 68.8 | −1.9 | ND | ND | CAT-2200 | Pertuzumab |
| 11103-11186 | 0.36 | −0.12 | 7.40 | 0.04 | 72.7 | −4.3 | 67.7 | −14.3 | 68.8 | −1.9 | ND | ND | CAT-2200 | Pertuzumab |
| 11104-11190 | 0.36 | −0.12 | ND | ND | 72.7 | −4.3 | 76.0 | −6.0 | 68.8 | −1.9 | ND | ND | CAT-2200 | Pertuzumab |
| 11105-11195 | 0.36 | −0.12 | ND | ND | 72.7 | −4.3 | ND | ND | 68.8 | −1.9 | ND | ND | CAT-2200 | Pertuzumab |
| 11106-11225 | 0.36 | −0.12 | ND | ND | 72.7 | −4.3 | ND | ND | 68.8 | −1.9 | ND | ND | CAT-2200 | Pertuzumab |
| 11106-11229 | 0.36 | −0.12 | 7.71 | 0.02 | 72.7 | −4.3 | 77.5 | −4.5 | 68.8 | −1.9 | ND | ND | CAT-2200 | Pertuzumab |
| 11107-11197 | 0.36 | −0.12 | 8.22 | −0.01 | 72.7 | −4.3 | 80.7 | −1.3 | 68.8 | −1.9 | ND | ND | CAT-2200 | Pertuzumab |
| 11108-11227 | 0.36 | −0.12 | ND | ND | 72.7 | −4.3 | ND | ND | 68.8 | −1.9 | ND | ND | CAT-2200 | Pertuzumab |
| 11108-11231 | 0.36 | −0.12 | 7.80 | 0.01 | 72.7 | −4.3 | 77.2 | −4.8 | 68.8 | −1.9 | ND | ND | CAT-2200 | Pertuzumab |
| 11124-11202 | 0.24 | 0.06 | 8.09 | 0.00 | 71.8 | −5.2 | 80.0 | −2.0 | ND | ND | 76.5 | −2.2 | CAT-2200 | Pertuzumab |
| 11125-11207 | 0.24 | 0.06 | 8.11 | 0.00 | 71.8 | −5.2 | 80.8 | −1.2 | ND | ND | 77.5 | −1.2 | CAT-2200 | Pertuzumab |
| 11140-11202 | 0.23 | 0.08 | 8.09 | 0.00 | 71.8 | −5.2 | 80.0 | −2.0 | 68.1 | −2.6 | 76.5 | −2.2 | CAT-2200 | Pertuzumab |
| 11141-11207 | 0.23 | 0.08 | 8.11 | 0.00 | 71.8 | −5.2 | 80.8 | −1.2 | 68.1 | −2.6 | 77.5 | −1.2 | CAT-2200 | Pertuzumab |
| 11142-11182 | 0.23 | 0.08 | 7.30 | 0.04 | 71.8 | −5.2 | 75.8 | −6.2 | 68.1 | −2.6 | ND | ND | CAT-2200 | Pertuzumab |
| 11143-11187 | 0.23 | 0.08 | 7.40 | 0.04 | 71.8 | −5.2 | 67.7 | −14.3 | 68.1 | −2.6 | ND | ND | CAT-2200 | Pertuzumab |
| 11144-11192 | 0.23 | 0.08 | ND | ND | 71.8 | −5.2 | 76.0 | −6.0 | 68.1 | −2.6 | ND | ND | CAT-2200 | Pertuzumab |
| 11148-11183 | 0.34 | −0.09 | 7.30 | 0.04 | 71.0 | −6.0 | 75.8 | −6.2 | ND | ND | ND | ND | CAT-2200 | Pertuzumab |
| 11149-11188 | 0.34 | −0.09 | 7.40 | 0.04 | 71.0 | −6.0 | 67.7 | −14.3 | ND | ND | ND | ND | CAT-2200 | Pertuzumab |
| 11150-11193 | 0.34 | −0.09 | ND | ND | 71.0 | −6.0 | 76.0 | −6.0 | ND | ND | ND | ND | CAT-2200 | Pertuzumab |
| 11156-11274 | 0.22 | 0.08 | 7.19 | 0.05 | 71.3 | −5.8 | 81.2 | −0.8 | ND | ND | ND | ND | CAT-2200 | Pertuzumab |
| 11157-11279 | 0.22 | 0.08 | 7.00 | 0.06 | 71.3 | −5.8 | 81.3 | −0.7 | ND | ND | 77.9 | −0.8 | CAT-2200 | Pertuzumab |
| 11158-11203 | 0.22 | 0.08 | 8.09 | 0.00 | 71.3 | −5.8 | 80.0 | −2.0 | ND | ND | 76.5 | −2.2 | CAT-2200 | Pertuzumab |
| 11159-11211 | 0.22 | 0.08 | ND | ND | 71.3 | −5.8 | ND | ND | ND | ND | ND | ND | CAT-2200 | Pertuzumab |
| 11159-11217 | 0.22 | 0.08 | 7.98 | 0.00 | 71.3 | −5.8 | 75.5 | −6.5 | ND | ND | ND | ND | CAT-2200 | Pertuzumab |
| 11160-11208 | 0.22 | 0.08 | 8.11 | 0.00 | 71.3 | −5.8 | 80.8 | −1.2 | ND | ND | 77.5 | −1.2 | CAT-2200 | Pertuzumab |
| 11161-11214 | 0.22 | 0.08 | ND | ND | 71.3 | −5.8 | ND | ND | ND | ND | ND | ND | CAT-2200 | Pertuzumab |
| 11161-11220 | 0.22 | 0.08 | 8.06 | 0.00 | 71.3 | −5.8 | 77.0 | −5.0 | ND | ND | 74.3 | −4.4 | CAT-2200 | Pertuzumab |
| 11162-11184 | 0.22 | 0.08 | 7.30 | 0.04 | 71.3 | −5.8 | 75.8 | −6.2 | ND | ND | ND | ND | CAT-2200 | Pertuzumab |
| 11163-11194 | 0.22 | 0.08 | ND | ND | 71.3 | −5.8 | 76.0 | −6.0 | ND | ND | ND | ND | CAT-2200 | Pertuzumab |
| 11164-11196 | 0.22 | 0.08 | ND | ND | 71.3 | −5.8 | ND | ND | ND | ND | ND | ND | CAT-2200 | Pertuzumab |
| 11165-11226 | 0.22 | 0.08 | ND | ND | 71.3 | −5.8 | ND | ND | ND | ND | ND | ND | CAT-2200 | Pertuzumab |
| 11165-11230 | 0.22 | 0.08 | 7.71 | 0.02 | 71.3 | −5.8 | 77.5 | −4.5 | ND | ND | ND | ND | CAT-2200 | Pertuzumab |
| 11166-11198 | 0.22 | 0.08 | 8.22 | −0.01 | 71.3 | −5.8 | 80.7 | −1.3 | ND | ND | ND | ND | CAT-2200 | Pertuzumab |
| 11167-11228 | 0.22 | 0.08 | ND | ND | 71.3 | −5.8 | ND | ND | ND | ND | ND | ND | CAT-2200 | Pertuzumab |
| 11167-11232 | 0.22 | 0.08 | 7.80 | 0.01 | 71.3 | −5.8 | 77.2 | −4.8 | ND | ND | ND | ND | CAT-2200 | Pertuzumab |

TABLE 6B
Antigen binding and thermal stability assessment of the K-K-derived K-L designs that passed the LCCA performance cut-off criteria
K-K-derived K-L designs

| | | | | Column | | Column | | Column | | Column | | Column | | |
|---|---|---|---|---|---|---|---|---|---|---|---|---|---|---|
| | | Column | | 5 | | 7 | | 9 | | 11 | | 13 | | |
| | Column | 3 | | Change | Column | Change | Column | Change | Column | Change | Column | Change | | |
| | 2 | Change | | in | 6 | in DSF | 8 | in DSF | 10 | in DSC | 12 | in DSC | | |
| | KD of | in | Column | KD of | DSF | values | DSF | values | DSC | values | DSC | values | | |
| | H1L1 | KD of | 4 | H2L2 | values | of | values | of | values | of | values | of | | |
| | Fab | H1L1 | KD of | Fab | of | H1L1 | of | H2L2 | of | H1L1 | of | H2L2 | Column | Column |
| Column 1 | hetero- | Fab | H2L2 | vs | H1L1 | Fab | H2L2 | Fab | H1L1 | Fab | H2L2 | Fab | 14 | 15 |
| Unique | dimer | vs wild | Fab | wild | Fab | vs wild | Fab | vs wild | Fab | vs wild | Fab | vs wild | H1L1 | H2L2 |
| identifier | (nM) | type | (nM) | type | (° C.) | type | (° C.) | type | (° C.) | type | (° C.) | type | Fab | Fab |
| 10621-10731 | 0.27 | 0.00 | 6.75 | 0.08 | 77.0 | 0.0 | 80.5 | −1.5 | 70.7 | 0.0 | ND | ND | CAT- | Pertu- |
| | | | | | | | | | | | | | 2200 | zumab |
| 10621-10733 | 0.27 | 0.00 | 5.72 | 0.15 | 77.0 | 0.0 | 83.8 | 1.8 | 70.7 | 0.0 | 79.4 | 0.7 | CAT- | Pertu- |
| | | | | | | | | | | | | | 2200 | zumab |
| 10622-10736 | 0.27 | 0.00 | ND | ND | 77.0 | 0.0 | ND | ND | 70.7 | 0.0 | ND | ND | CAT- | Pertu- |
| | | | | | | | | | | | | | 2200 | zumab |
| 10622-10742 | 0.27 | 0.00 | ND | ND | 77.0 | 0.0 | ND | ND | 70.7 | 0.0 | ND | ND | CAT- | Pertu- |
| | | | | | | | | | | | | | 2200 | zumab |
| 10623-10739 | 0.27 | 0.00 | 6.39 | 0.10 | 77.0 | 0.0 | 81.3 | −0.7 | 70.7 | 0.0 | ND | ND | CAT- | Pertu- |
| | | | | | | | | | | | | | 2200 | zumab |
| 10623-10745 | 0.27 | 0.00 | 8.08 | 0.00 | 77.0 | 0.0 | 82.5 | 0.5 | 70.7 | 0.0 | 78.3 | −0.4 | CAT- | Pertu- |
| | | | | | | | | | | | | | 2200 | zumab |
| 10624-10700 | ND | ND | ND | ND | ND | ND | ND | ND | ND | ND | ND | ND | CAT- | Pertu- |
| | | | | | | | | | | | | | 2200 | zumab |
| 10625-10701 | ND | ND | ND | ND | ND | ND | ND | ND | ND | ND | ND | ND | CAT- | Pertu- |
| | | | | | | | | | | | | | 2200 | zumab |
| 10629-10695 | ND | ND | ND | ND | ND | ND | 79.8 | −2.2 | ND | ND | ND | ND | CAT- | Pertu- |
| | | | | | | | | | | | | | 2200 | zumab |
| 10631-10722 | 0.19 | 0.15 | ND | ND | 73.7 | −3.3 | ND | ND | ND | ND | 78.0 | −0.7 | CAT- | Pertu- |
| | | | | | | | | | | | | | 2200 | zumab |
| 10632-10724 | 0.19 | 0.15 | NB | −2.09 | 73.7 | −3.3 | 74.0 | −7.9 | ND | ND | 77.8 | −0.9 | CAT- | Pertu- |
| | | | | | | | | | | | | | 2200 | zumab |
| 10640-10713 | 0.23 | 0.08 | 8.28 | −0.01 | 73.3 | −3.7 | 80.8 | −1.1 | 69.1 | −1.6 | 78.2 | −0.5 | CAT- | Pertu- |
| | | | | | | | | | | | | | 2200 | zumab |
| 10652-10702 | 0.28 | −0.01 | 7.78 | 0.02 | 73.0 | −4.0 | 81.3 | −0.7 | ND | ND | ND | ND | CAT- | Pertu- |
| | | | | | | | | | | | | | 2200 | zumab |
| 10652-10734 | 0.28 | −0.01 | 7.65 | 0.02 | 73.0 | −4.0 | 83.2 | 1.3 | ND | ND | ND | ND | CAT- | Pertu- |
| | | | | | | | | | | | | | 2200 | zumab |
| 10652-10755 | 0.28 | −0.01 | 6.98 | 0.06 | 73.0 | −4.0 | 81.7 | −0.3 | ND | ND | 78.4 | −0.3 | CAT- | Pertu- |
| | | | | | | | | | | | | | 2200 | zumab |
| 10656-10758 | ND | ND | ND | ND | ND | ND | ND | ND | ND | ND | ND | ND | CAT- | Pertu- |
| | | | | | | | | | | | | | 2200 | zumab |
| 10657-10760 | ND | ND | ND | ND | ND | ND | ND | ND | ND | ND | ND | ND | CAT- | Pertu- |
| | | | | | | | | | | | | | 2200 | zumab |
| 10658-10720 | ND | ND | ND | ND | ND | ND | ND | ND | ND | ND | 78.0 | −0.7 | CAT- | Pertu- |
| | | | | | | | | | | | | | 2200 | zumab |
| 10659-10720 | ND | ND | ND | ND | ND | ND | ND | ND | ND | ND | 78.0 | −0.7 | CAT- | Pertu- |
| | | | | | | | | | | | | | 2200 | zumab |
| 10662-10731 | 0.29 | −0.02 | 6.75 | 0.08 | 75.0 | −2.0 | 80.5 | −1.5 | ND | ND | ND | ND | CAT- | Pertu- |
| | | | | | | | | | | | | | 2200 | zumab |
| 10662-10733 | 0.29 | −0.02 | 5.72 | 0.15 | 75.0 | −2.0 | 83.8 | 1.8 | ND | ND | 79.4 | 0.7 | CAT- | Pertu- |
| | | | | | | | | | | | | | 2200 | zumab |
| 10664-10722 | 0.29 | −0.03 | ND | ND | 77.3 | 0.3 | ND | ND | 70.7 | 0.0 | 78.0 | −0.7 | CAT- | Pertu- |
| | | | | | | | | | | | | | 2200 | zumab |
| 10665-10724 | 0.29 | −0.03 | NB | −2.09 | 77.3 | 0.3 | 74.0 | −7.9 | 70.7 | 0.0 | 77.8 | −0.9 | CAT- | Pertu- |
| | | | | | | | | | | | | | 2200 | zumab |
| 10671-10751 | ND | ND | ND | ND | ND | ND | ND | ND | ND | ND | ND | ND | CAT- | Pertu- |
| | | | | | | | | | | | | | 2200 | zumab |
| 10674-10749 | 0.21 | 0.12 | 6.09 | 0.12 | 74.5 | −2.5 | 81.0 | −1.0 | ND | ND | ND | ND | CAT- | Pertu- |
| | | | | | | | | | | | | | 2200 | zumab |
| 10676-10750 | ND | ND | ND | ND | ND | ND | ND | ND | ND | ND | ND | ND | CAT- | Pertu- |
| | | | | | | | | | | | | | 2200 | zumab |
| 10677-10751 | ND | ND | ND | ND | ND | ND | ND | ND | ND | ND | ND | ND | CAT- | Pertu- |
| | | | | | | | | | | | | | 2200 | zumab |
| 10678-10737 | ND | ND | ND | ND | ND | ND | ND | ND | ND | ND | ND | ND | CAT- | Pertu- |
| | | | | | | | | | | | | | 2200 | zumab |
| 10678-10743 | ND | ND | ND | ND | ND | ND | ND | ND | ND | ND | ND | ND | CAT- | Pertu- |
| | | | | | | | | | | | | | 2200 | zumab |
| 10679-10738 | ND | ND | ND | ND | ND | ND | ND | ND | ND | ND | ND | ND | CAT- | Pertu- |
| | | | | | | | | | | | | | 2200 | zumab |
| 10679-10744 | ND | ND | ND | ND | ND | ND | ND | ND | ND | ND | ND | ND | CAT- | Pertu- |
| | | | | | | | | | | | | | 2200 | zumab |
| 10680-10740 | 0.39 | −0.16 | 6.39 | 0.10 | 72.0 | −5.0 | 81.3 | −0.7 | 74.2 | 3.5 | ND | ND | CAT- | Pertu- |
| | | | | | | | | | | | | | 2200 | zumab |
| 10680-10746 | 0.39 | −0.16 | 8.08 | 0.00 | 72.0 | −5.0 | 82.5 | 0.5 | 74.2 | 3.5 | 78.3 | −0.4 | CAT- | Pertu- |
| | | | | | | | | | | | | | 2200 | zumab |
| 10681-10741 | 0.35 | −0.11 | 6.39 | 0.10 | 71.5 | −5.5 | 81.3 | −0.7 | ND | ND | ND | ND | CAT- | Pertu- |
| | | | | | | | | | | | | | 2200 | zumab |
| 10681-10747 | 0.35 | −0.11 | 8.08 | 0.00 | 71.5 | −5.5 | 82.5 | 0.5 | ND | ND | 78.3 | −0.4 | CAT- | Pertu- |
| | | | | | | | | | | | | | 2200 | zumab |
| 10682-10718 | ND | ND | 7.43 | 0.04 | ND | ND | 81.7 | −0.3 | ND | ND | ND | ND | CAT- | Pertu- |
| | | | | | | | | | | | | | 2200 | zumab |
| 10683-10720 | ND | ND | ND | ND | ND | ND | ND | ND | ND | ND | 78.0 | −0.7 | CAT- | Pertu- |
| | | | | | | | | | | | | | 2200 | zumab |
| 10684-10706 | 0.26 | 0.03 | 7.28 | 0.04 | 75.2 | −1.8 | 81.5 | −0.5 | 69.6 | −1.1 | 77.2 | −1.5 | CAT- | Pertu- |
| | | | | | | | | | | | | | 2200 | zumab |
| 10685-10719 | 0.22 | 0.09 | 7.43 | 0.04 | 75.0 | −2.0 | 81.7 | −0.3 | 70.3 | −0.4 | ND | ND | CAT- | Pertu- |
| | | | | | | | | | | | | | 2200 | zumab |
| 10685-10726 | 0.22 | 0.09 | 7.24 | 0.05 | 75.0 | −2.0 | 80.3 | −1.7 | 70.3 | −0.4 | 77.4 | −1.3 | CAT- | Pertu- |
| | | | | | | | | | | | | | 2200 | zumab |
| 10687-10722 | 0.22 | 0.09 | ND | ND | 75.0 | −2.0 | ND | ND | 70.3 | −0.4 | 78.0 | −0.7 | CAT- | Pertu- |
| | | | | | | | | | | | | | 2200 | zumab |
| 10688-10724 | 0.22 | 0.09 | NB | −2.09 | 75.0 | −2.0 | 74.0 | −7.9 | 70.3 | −0.4 | 77.8 | −0.9 | CAT- | Pertu- |
| | | | | | | | | | | | | | 2200 | zumab |
| 10689-10707 | 0.22 | 0.09 | 7.28 | 0.04 | 75.0 | −2.0 | 81.5 | −0.5 | 70.3 | −0.4 | 77.2 | −1.5 | CAT- | Pertu- |
| | | | | | | | | | | | | | 2200 | zumab |
| 10690-10727 | 0.24 | 0.05 | 7.24 | 0.05 | 73.5 | −3.5 | 80.3 | −1.7 | ND | ND | 77.4 | −1.3 | CAT- | Pertu- |
| | | | | | | | | | | | | | 2200 | zumab |
| 10621-10731 | 0.27 | 0.00 | 6.75 | 0.08 | 77.0 | 0.0 | 80.5 | −1.5 | 70.7 | 0.0 | ND | ND | CAT- | Pertu- |
| | | | | | | | | | | | | | 2200 | zumab |

TABLE 7A
Second K-L Design Library
Unique identifier

| Unique | H1 mutation | | | | | |
|---|---|---|---|---|---|---|
| identifier | (Kabat) | L1 mutation (Kabat) | H2 mutations (Kabat) | L2 mutation (Kabat) | H1L1 Fab | H2L2 Fab |
| 12944-13093 | Q179K | K129T_S180E | L143E_K145T_Q179E | Q124R_T178R | CAT-2200 | Pertuzumab |
| 12958-13064 | S186R | K129T_S180E | L143E_K145T | Q124R_Q160K_T178R | CAT-2200 | Pertuzumab |
| 12958-13106 | S186R | K129T_S180E | L143E_K145T_Q179E_S188L | Q124R_Q160K_T178R | CAT-2200 | Pertuzumab |
| 12958-13469 | S186R | K129T_S180E | L143E_K145T_S188L | Q124R_Q160K_T178R | CAT-2200 | Pertuzumab |
| 12961-13108 | S186R | K129T_S180E | L143E_K145T_Q179E_S188L | Q124R_T178R | CAT-2200 | Pertuzumab |
| 12961-13471 | S186R | K129T_S180E | L143E_K145T_S188L | Q124R_T178R | CAT-2200 | Pertuzumab |
| 13200-13421 | A125R_S186R | S122D_K129T_S180E | L143E_K145T | Q124R_L135W_Q160K_T178R | CAT-2200 | Pertuzumab |
| 13202-13426 | A125R_S186R | S122D_K129T_S180E | L143E_K145T_K228D | S121K_Q124R_Q160K_T178R | CAT-2200 | Pertuzumab |
| 13203-13427 | A125R_S186R | S122D_K129T_S180E | L143E_K145T_K228D | S121K_Q124R_T178R | CAT-2200 | Pertuzumab |
| 13248-13475 | F174G_S186R | T116F_K129T_S176F_S180E | L143E_K145T_V190F | Q124R_L135A_Q160K_T178R | CAT-2200 | Pertuzumab |
| 13309-13443 | Q179K | K129T_S180E | L143E_K145T_Q179E | Q124R_T129K_Q160K_T178R | CAT-2200 | Pertuzumab |
| 13322-13425 | S186R | K129T_S180E | L143E_K145T | Q124R_T129K_Q160K_T178R | CAT-2200 | Pertuzumab |
| 13204-13351 | A125R_S186R | S122D_K129T_S180E | K145T_Q179E_K228D | S121K_S131K | CAT-2200 | Pertuzumab |
| 13205-13353 | A125R_S186R | S122D_K129T_S180E | K145T_Q179E_K228D | S121K_T129K_S131K | CAT-2200 | Pertuzumab |
| 13196-13351 | A125R_Q179K | S122D_K129T_S180E | K145T_Q179E_K228D | S121K_S131K | CAT-2200 | Pertuzumab |
| 13197-13353 | A125R_Q179K | S122D_K129T_S180E | K145T_Q179E_K228D | S121K_T129K_S131K | CAT-2200 | Pertuzumab |
| 13250-13355 | F174G_S186R | T116F_K129T_S176F_S180E | K145T_Q179E_S188F_V190F | S131K_L135A | CAT-2200 | Pertuzumab |
| 13277-13420 | L143A_S186R | K129T_V133W_S180E | L124W_L143E_K145T | Q124K_V133A_T178R | CAT-2200 | Pertuzumab |
| 13319-13473 | S186R | K129T_S176A_Y178W_S180E | L143E_K145T_5188W | Q124K_S176A_T178R | CAT-2200 | Pertuzumab |
| 13188-13421 | A125R_Q179K | S122D_K129T_S180E | L143E_K145T | Q124R_L135W_Q160K_T178R | CAT-2200 | Pertuzumab |
| 13190-13422 | A125R_Q179K | S122D_K129T_S180E | L143E_K145T | Q124R_L135W_T178R | CAT-2200 | Pertuzumab |
| 13242-13475 | F174G_Q179K | T116F_K129T_S176F_S180E | L143E_K145T_V190F | Q124R_L135A_Q160K_T178R | CAT-2200 | Pertuzumab |
| 13192-13426 | A125R_Q179K | S122D_K129T_S180E | L143E_K145T_K228D | S121K_Q124R_Q160K_T178R | CAT-2200 | Pertuzumab |
| 13194-13427 | A125R_Q179K | S122D_K129T_S180E | L143E_K145T_K228D | S121K_Q124R_T178R | CAT-2200 | Pertuzumab |
| 13317-13423 | Q179K_V190K | K129T_V133D_L1355_S180E | L143E_K145T | Q124R_Q160K_T178R | CAT-2200 | Pertuzumab |
| 13188-13430 | A125R_Q179K | S122D_K129T_S180E | L143E_K145T_Q179E | Q124R_L135W_Q160K_T178R | CAT-2200 | Pertuzumab |
| 13200-13430 | A125R_S186R | S122D_K129T_S180E | L143E_K145T_Q179E | Q124R_L135W_Q160K_T178R | CAT-2200 | Pertuzumab |
| 13246-13463 | F174G_S186R | T116F_K129T_S176F_S180E | L143E_K145T_Q179E_V190F | Q124K_L135A_T178R | CAT-2200 | Pertuzumab |
| 13238-13463 | F174G_Q179K | T116F_K129T_S176F_S180E | L143E_K145T_Q179E_V190F | Q124K_L135A_T178R | CAT-2200 | Pertuzumab |
| 13248-13465 | F174G_S186R | T116F_K129T_S176F_S180E | L143E_K145T_Q179E_V190F | Q124R_L135A_Q160K_T178R | CAT-2200 | Pertuzumab |
| 13242-13465 | F174G_Q179K | T116F_K129T_S176F_S180E | L143E_K145T_Q179E_V190F | Q124R_L135A_Q160K_T178R | CAT-2200 | Pertuzumab |
| 13246-13459 | F174G_S186R | T116F_K129T_S176F_S180E | L143E_K145T_Q179E_S188F_V190F | Q124K_L135A_T178R | CAT-2200 | Pertuzumab |
| 13238-13459 | F174G_Q179K | T116F_K129T_S176F_S180E | L143E_K145T_Q179E_S188F_V190F | Q124K_L135A_T178R | CAT-2200 | Pertuzumab |
| 13192-13452 | A125R_Q179K | S122D_K129T_S180E | L143E_K145T_Q179E_K228D | S121K_Q124R_Q160K_T178R | CAT-2200 | Pertuzumab |
| 13194-13456 | A125R_Q179K | S122D_K129T_S180E | L143E_K145T_Q179E_K228D | S121K_Q124R_T178R | CAT-2200 | Pertuzumab |
| 13202-13452 | A125R_S186R | S122D_K129T_S180E | L143E_K145T_Q179E_K228D | S121K_Q124R_Q160K_T178R | CAT-2200 | Pertuzumab |
| 13203-13456 | A125R_S186R | S122D_K129T_S180E | L143E_K145T_Q179E_K228D | S121K_Q124R_T178R | CAT-2200 | Pertuzumab |
| 13254-13508 | K145T_S188E | Y178K | S186R_S188K | Q124E_Q160E_S176D_T178D | CAT-2200 | Pertuzumab |
| 13255-13509 | K145T_S188E | Y178K | S186R_S188K | Q124E_Q160E_S176D_T178D_T180E | CAT-2200 | Pertuzumab |
| 13262-13481 | K145T_S188E | Y178K | L143K_S188K | Q124E_V133D_S176E_T180E | CAT-2200 | Pertuzumab |
| 13261-13480 | K145T_S188E | Y178K | L143K_S188K | Q124E_V133D_S176E_T178E_T180E | CAT-2200 | Pertuzumab |
| 13258-13504 | K145T_S188E | Y178K | Q179K_S188K | Q124E_S176E_T180E | CAT-2200 | Pertuzumab |
| 13256-13502 | K145T_S188E | Y178K | Q179K_S188K | Q124E_S176E_T178D_T180E | CAT-2200 | Pertuzumab |
| 13257-13503 | K145T_S188E | Y178K | Q179K_S188K | Q124E_S176E_T178E_T180E | CAT-2200 | Pertuzumab |
| 13269-13403 | K145T_S188E | Y178K | L124R_L143K | Q124E_V133G_S176D_T180E | CAT-2200 | Pertuzumab |
| 13268-13401 | K145T_S188E | Y178K | L124R_L143K | Q124E_V133G_S176D_T178E_T180E | CAT-2200 | Pertuzumab |
| 12878-11240 | K145T_S188E | Y178K | L124R | V133G_S176D | CAT-2200 | Pertuzumab |
| 10798-13036 | K145T_S188E | Y178K | L124R | V133G_S176D_T180E | CAT-2200 | Pertuzumab |
| 13265-13392 | K145T_S188E | Y178K | L124K_S188K | Q124E_V133G_S176D_T178D | CAT-2200 | Pertuzumab |
| 13265-13419 | K145T_S188E | Y178K | L124R_S188K | Q124E_V133G_S176D_T178D | CAT-2200 | Pertuzumab |
| 13212-13396 | A139W_K145T_S188E | Y178K | L124R | V133G_L135W_S176D_T180E | CAT-2200 | Pertuzumab |
| 13211-13395 | A139W_K145T_S188E | Y178K | L124R | V133G_L135W_S176D | CAT-2200 | Pertuzumab |
| 13168-13397 | A125R_K145T_S188E | S122D_Y178K | L124R_K228D | S121K_V133G_S176D | CAT-2200 | Pertuzumab |
| 13296-13399 | L143E_K145T_Q179E | E124Q_T131R | L124R_L143K | Q124E_V133G_S176D | CAT-2200 | Pertuzumab |
| 13297-13402 | L143E_K145T_Q179E | E124Q_T131R | L124R_L143K | Q124E_V133G_S176D_T180E | CAT-2200 | Pertuzumab |
| 13264-13400 | K145T_S188E | Y178K | L124R_L143K | Q124E_V133G_S176D | CAT-2200 | Pertuzumab |
| 13332-13360 | S188K | K129T_S176E_Y178E | K145T_S186E | S131K_S176K | CAT-2200 | Pertuzumab |
| 13328-13359 | S188K | K129T_S176D_Y178T | K145T_S186E | S131K_S176K | CAT-2200 | Pertuzumab |
| 13335-13361 | S188K | K129T_Y178D | K145T_S186E | S131K_S176K | CAT-2200 | Pertuzumab |
| 13337-13362 | S188K | K129T_Y178E | K145T_S186E | S131K_S176K | CAT-2200 | Pertuzumab |
| 13237-13358 | A139W_S188K | K129T_S176E_Y178E | K145T_S186E | S131K_L135W_S176K | CAT-2200 | Pertuzumab |
| 13235-13357 | A139W_S188K | K129T_S176D_Y178T | K145T_S186E | S131K_L135W_S176K | CAT-2200 | Pertuzumab |
| 13209-13364 | A125R_S188K | S122D_K129T_S176E_Y178E | K145T_S186E_K228D | S121K_S131K_S176K | CAT-2200 | Pertuzumab |
| 13207-13363 | A125R_S188K | S122D_K129T_S176D_Y178T | K145T_S186E_K228D | S121K_S131K_S176K | CAT-2200 | Pertuzumab |
| 13210-13510 | A139W_K145T_S188E | Y178K | S188K | L135W_S176E_T178E | CAT-2200 | Pertuzumab |
| 13167-13514 | A125R_K145T_S188E | S122D_Y178K | S188K_K228D | S121K_S176E_T178E | CAT-2200 | Pertuzumab |
| 13294-13492 | L143E_K145T_Q179E | E124Q_T131R | L143R_S188K | Q124E_V133D_T178E | CAT-2200 | Pertuzumab |
| 13292-13489 | L143E_K145T_Q179E | E124Q_T131R | L143R_S188K | Q124E_V133D_S176D_T178E | CAT-2200 | Pertuzumab |
| 13263-13493 | K145T_S188E | Y178K | L143R_S188K | Q124E_V133D_T178E | CAT-2200 | Pertuzumab |
| 13259-13491 | K145T_S188E | Y178K | L143R_S188K | Q124E_V133D_S176D_T178E | CAT-2200 | Pertuzumab |
| 13212-13415 | A139W_K145T_S188E | Y178K | L124R_S186R | V133G_L135W_S176D_T180E | CAT-2200 | Pertuzumab |
| 13169-13418 | A125R_K145T_S188E | S122D_Y178K | L124R_S186R_K228D | S121K_V133G_S176D_T180E | CAT-2200 | Pertuzumab |
| 13331-13447 | S188K | K129T_S176E_Y178E | L143E_K145T_Q179E | Q124R_T178R | CAT-2200 | Pertuzumab |
| 13327-13446 | S188K | K129T_S176D_Y178T | L143E_K145T_Q179E | Q124R_T178R | CAT-2200 | Pertuzumab |
| 13334-13449 | S188K | K129T_Y178D | L143E_K145T_Q179E | Q124R_T178R | CAT-2200 | Pertuzumab |
| 13336-13450 | S188K | K129T_Y178E | L143E_K145T_Q179E | Q124R_T178R | CAT-2200 | Pertuzumab |
| 13236-13433 | A139W_S188K | K129T_S176E_Y178E | L143E_K145T_Q179E | Q124R_L135W_T178R | CAT-2200 | Pertuzumab |
| 13234-13432 | A139W_S188K | K129T_S176D_Y178T | L143E_K145T_Q179E | Q124R_L135W_T178R | CAT-2200 | Pertuzumab |
| 13208-13455 | A125R_S188K | S122D_K129T_S176E_Y178E | L143E_K145T_Q179E_K228D | S121K_Q124R_T178R | CAT-2200 | Pertuzumab |
| 13206-13454 | A125R_S188K | S122D_K129T_S176D_Y178T | L143E_K145T_Q179E_K228D | S121K_Q124R_T178R | CAT-2200 | Pertuzumab |
| 13333-13387 | S188K | K129T_S176E_Y178E | L124E_K145T_Q179E | S131R_V133G_S176R | CAT-2200 | Pertuzumab |
| 13295-13484 | L143E_K145T_Q179E | E124Q_T131R | L143R | Q124E_V133D_T180D | CAT-2200 | Pertuzumab |
| 13282-13484 | L143D_K145T | E124Q_T131R | L143R | Q124E_V133D_T180D | CAT-2200 | Pertuzumab |
| 13218-13482 | A139W_L143D_K145T | E124Q_T131R | L143R | Q124E_V133D_L135W | CAT-2200 | Pertuzumab |
| 13219-13483 | A139W_L143D_K145T | E124Q_T131R | L143R | Q124E_V133D_L135W_T180D | CAT-2200 | Pertuzumab |
| 13175-13486 | A125R_L143D_K145T | S122D_E124Q_T131R | L143R_K228D | S121K_Q124E_V133D | CAT-2200 | Pertuzumab |
| 13176-13487 | A125R_L143D_K145T | S122D_E124Q_T131R | L143R_K228D | S121K_Q124E_V133D_T180D | CAT-2200 | Pertuzumab |
| 13280-13488 | L143D_K145T | E124Q_T131R | L143R_S188K | Q124E_V133D_S176D_T178D | CAT-2200 | Pertuzumab |
| 13281-13489 | L143D_K145T | E124Q_T131R | L143R_S188K | Q124E_V133D_S176D_T178E | CAT-2200 | Pertuzumab |
| 13291-13488 | L143E_K145T_Q179E | E124Q_T131R | L143R_S188K | Q124E_V133D_S176D_T178D | CAT-2200 | Pertuzumab |
| 13287-13494 | L143D_K145T_F174G | T116F_E124Q_T131R_S176F | L143R_V190F | Q124E_V133D_L135A | CAT-2200 | Pertuzumab |
| 13214-13405 | A139W_L143D_K145T | E124Q_T131K | L124R_Q179K | V133G_L135W_S176D_T180E | CAT-2200 | Pertuzumab |
| 13221-13406 | A139W_L143D_K145T | E124Q_T131R | L124R_Q179K | V133G_L135W_S176D_T180E | CAT-2200 | Pertuzumab |
| 13214-13410 | A139W_L143D_K145T | E124Q_T131K | L124R_S186K | V133G_L135W_S176D_T180E | CAT-2200 | Pertuzumab |
| 13221-13411 | A139W_L143D_K145T | E124Q_T131R | L124R_S186K | V133G_L135W_S176D_T180E | CAT-2200 | Pertuzumab |
| 13285-13414 | L143D_K145T_F174G | T116F_E124Q_T131K_S176F | L124R_S186K_V190F | V133G_L135A_S176D_T180E | CAT-2200 | Pertuzumab |
| 13171-13416 | A125R_L143D_K145T | S122D_E124Q_T131K | L124R_S186R_K228D | S121K_V133G_S176D_T180E | CAT-2200 | Pertuzumab |
| 13178-13417 | A125R_L143D_K145T | S122D_E124Q_T131R | L124R_S186R_K228D | S121K_V133G_S176D_T180E | CAT-2200 | Pertuzumab |
| 13220-13511 | A139W_L143D_K145T | E124Q_T131R | S188K | S131D_V133G_L135W_T178F | CAT-2200 | Pertuzumab |
| 13177-13513 | A125R_L143D_K145T | S122D_E124Q_T131R | S188K_K228D | S121K_S131D_V133G_T178F | CAT-2200 | Pertuzumab |
| 13288-13516 | L143E_K145T_F174G_Q179E | T116F_E124Q_T131R_S176F | S188K_V190F | V133I_L135A_S176D_T178E | CAT-2200 | Pertuzumab |
| 13223-13512 | A139W_L143E_K145T_Q179E | E124Q_T131R | S188K | V133I_L135W_S176D_T178E | CAT-2200 | Pertuzumab |
| 13180-13515 | A125R_L143E_K145T_Q179E | S122D_E124Q_T131R | S188K_K228D | S121K_V133I_S176D_T178E | CAT-2200 | Pertuzumab |
| 13298-13439 | L143K | K129T_T131D_V133D | L143E_K145T_Q179E | Q124R_Q160R_T178R | CAT-2200 | Pertuzumab |
| 13300-13441 | L143K | K129T_T131E_V133D | L143E_K145T_Q179E | Q124R_Q160R_T178R | CAT-2200 | Pertuzumab |
| 13271-13439 | L124K_L143K | K129T_T131D_V133D | L143E_K145T_Q179E | Q124R_Q160R_T178R | CAT-2200 | Pertuzumab |
| 13273-13441 | L124K_L143K | K129T_T131E_V133D | L143E_K145T_Q179E | Q124R_Q160R_T178R | CAT-2200 | Pertuzumab |
| 13299-13379 | L143K | K129T_T131D_V133D | L124E_K145T_Q179E | S131K_V133G_S176R | CAT-2200 | Pertuzumab |
| 13301-13381 | L143K | K129T_T131E_V133D | L124E_K145T_Q179E | S131K_V133G_S176R | CAT-2200 | Pertuzumab |
| 13272-13379 | L124K_L143K | K129T_T131D_V133D | L124E_K145T_Q179E | S131K_V133G_S176R | CAT-2200 | Pertuzumab |
| 13274-13381 | L124K_L143K | K129T_T131E_V133D | L124E_K145T_Q179E | S131K_V133G_S176R | CAT-2200 | Pertuzumab |
| 13305-13448 | L143K_V190K | K129T_V133D_L135S | L143E_K145T_Q179E | Q124R_T178R | CAT-2200 | Pertuzumab |
| 13304-13466 | L143K_F174G | T116F_K129T_V133D_S176F | L143E_K145T_Q179E_V190F | Q124R_L135A_T178R | CAT-2200 | Pertuzumab |
| 13226-13434 | A139W_L143K | K129T_V133D | L143E_K145T_Q179E | Q124R_L135W_T178R | CAT-2200 | Pertuzumab |
| 13182-13458 | A125R_L143K | S122D_K129T_V133D | L143E_K145T_Q179E_K228D | S121K_Q124R_T178R | CAT-2200 | Pertuzumab |
| 13227-13383 | A139W_L143K | K129T_V133D | L124E_K145T_Q179E | S131R_V133G_L135W_S176R | CAT-2200 | Pertuzumab |
| 13184-13388 | A125R_L143K | S122D_K129T_V133D | L124E_K145T_Q179E_K228D | S121K_S131R_V133G_S176R | CAT-2200 | Pertuzumab |
| 13229-13428 | A139W_S186K | K129T_V133D_Y178T | L143E_K145T_Q179E | Q124R_L135W_Q160K_T178R | CAT-2200 | Pertuzumab |
| 13224-13428 | A139W_L143I_S186K | K129T_V133D_Y178T | L143E_K145T_Q179E | Q124R_L135W_Q160K_T178R | CAT-2200 | Pertuzumab |
| 13231-13386 | A139W_S186K_S188T | V133D_Y178T | L124E_K145T_Q179E | S131R_V133G_L135W_S176R | CAT-2200 | Pertuzumab |
| 13199-13391 | A125R_S186K_S188T | S122D_V133D_Y178T | L124E_K145T_Q179E_K228D | S121K_S131R_V133G_S176R | CAT-2200 | Pertuzumab |
| 13230-13384 | A139W_S186K | K129T_V133D_Y178T | L124E_K145T_Q179E | S131R_V133G_L135W_S176R | CAT-2200 | Pertuzumab |
| 13198-13389 | A125R_S186K | S122D_K129T_V133D_Y178T | L124E_K145T_Q179E_K228D | S121K_S131R_V133G_S176R | CAT-2200 | Pertuzumab |
| 13225-13384 | A139W_L143I_S186K | K129T_V133D_Y178T | L124E_K145T_Q179E | S131R_V133G_L135W_S176R | CAT-2200 | Pertuzumab |
| 13181-13389 | A125R_L143I_S186K | S122D_K129T_V133D_Y178T | L124E_K145T_Q179E_K228D | S121K_S131R_V133G_S176R | CAT-2200 | Pertuzumab |
| 13306-13375 | L143K_V190K | K129T_V133D_L135S | L124E_K145T | S131K_L135K | CAT-2200 | Pertuzumab |
| 13306-13373 | L143K_V190K | K129T_V133D_L135S | L124E_A139I_K145T_V190I | S131K_L135K | CAT-2200 | Pertuzumab |
| 13185-13374 | A125R_L143K_V190K | S122D_K129T_V133D_L135S | L124E_A139I_K145T_V190I_K228D | S121K_S131K_L135K | CAT-2200 | Pertuzumab |
| 13283-13507 | L143D_K145T_F174G | E124Q_T131K_V133S_S176F | S186K_V190F | Q124E_V133D_L135A | CAT-2200 | Pertuzumab |
| 13216-13505 | A139W_L143D_K145T | E124Q_T131K_V133S | S186K | Q124E_V133D_L135W | CAT-2200 | Pertuzumab |
| 13173-13506 | A125R_L143D_K145T | S122D_E124Q_T131K_V133S | S186K_K228D | S121K_Q124E_V133D | CAT-2200 | Pertuzumab |
| 13283-13478 | L143D_K145T_F174G | E124Q_T131K_V133S_S176F | L143I_S186K_V190F | Q124E_V133D_L135A | CAT-2200 | Pertuzumab |
| 13216-13476 | A139W_L143D_K145T | E124Q_T131K_V133S | L143I_S186K | Q124E_V133D_L135W | CAT-2200 | Pertuzumab |
| 13173-13477 | A125R_L143D_K145T | S122D_E124Q_T131K_V133S | L143I_S186K_K228D | S121K_Q124E_V133D | CAT-2200 | Pertuzumab |
| 13329-13366 | S188K | K129T_S176E_Y178E | L45P_L124E_K145T_Q179E | P44F_S131R_V133G_S176R | CAT-2200 | Pertuzumab |
| 13252-13369 | K145T_S188E | Y178K | L45P_L124R_Q179K | P44F_V133G_S176D_T180E | CAT-2200 | Pertuzumab |
| 13289-13368 | L143E_K145T_Q179E | E124Q_T131R | L45P_L124R_Q179K | P44F_V133G_S176D_T180E | CAT-2200 | Pertuzumab |
| 13338-13371 | S188K | S176E_Y178E | L45P_L143E_K145T_Q179E | P44F_Q124R_T178R | CAT-2200 | Pertuzumab |
| 13278-13372 | L143D_K145T | E124Q_T131R | L45P_L143R | P44F_Q124E_V133D | CAT-2200 | Pertuzumab |
| 13320-13370 | S186R | K129T_S180E | L45P_L143E_K145T_Q179E | P44F_Q124K_T178R | CAT-2200 | Pertuzumab |
| 13302-13367 | L143K | K129T_V133D | L45P_L124E_K145T_Q179E | P44F_S131R_V133G_S176R | CAT-2200 | Pertuzumab |
| 13330-13495 | S188K | K129T_S176E_Y178E | Q39E_L124E_K145T_Q179E | Q38R_S131R_V133G_S176R | CAT-2200 | Pertuzumab |
| 13253-13498 | K145T_S188E | Y178K | Q39E_L124R_Q179K | Q38R_V133G_S176D_T180E | CAT-2200 | Pertuzumab |
| 13290-13497 | L143E_K145T_Q179E | E124Q_T131R | Q39E_L124R_Q179K | Q38R_V133G_S176D_T180E | CAT-2200 | Pertuzumab |
| 13339-13500 | S188K | S176E_Y178E | Q39E_L143E_K145T_Q179E | Q38R_Q124R_T178R | CAT-2200 | Pertuzumab |
| 13279-13501 | L143D_K145T | E124Q_T131R | Q39E_L143R | Q38R_Q124E_V133D | CAT-2200 | Pertuzumab |
| 13321-13499 | S186R | K129T_S180E | Q39E_L143E_K145T_Q179E | Q38R_Q124K_T178R | CAT-2200 | Pertuzumab |
| 13303-13496 | L143K | K129T_V133D | Q39E_L124E_K145T_Q179E | Q38R_S131R_V133G_S176R | CAT-2200 | Pertuzumab |
| 12957-13061 | S186R | K129T_S180E | L143E_K145T | Q124K_T178R | CAT-2200 | Pertuzumab |
| 12906-13152 | L143D_K145T | E124Q_T131K_V133S | S186K | Q124E_V133D | CAT-2200 | Pertuzumab |
| 13318-13437 | S186K | K129T_V133D_Y178T | L143E_K145T_Q179E | Q124R_Q160K_T178R | CAT-2200 | Pertuzumab |

TABLE 7B
Second K-K derived K-L designs libarary

| Unique | H1 mutation | | | | | |
|---|---|---|---|---|---|---|
| identifier | (Kabat) | L1 mutation (Kabat) | H2 mutation (Kabat) | L2 mutation (Kabat) | H1L1 Fab | H2L2 Fab |
| 12938-13064 | Q179K | K129T_S180E | L143E_K145T | Q124R_Q160K_T178R | CAT-2200 | Pertuzumab |
| 13309-13425 | Q179K | K129T_S180E | L143E_K145T | Q124R_T129K_Q160K_T178R | CAT-2200 | Pertuzumab |
| 13314-13424 | Q179K | K129T_Y178E_S180E | L143E_K145T | Q124R_Q160K_T178R | CAT-2200 | Pertuzumab |
| 12938-13469 | Q179K | K129T_S180E | L143E_K145T_S188L | Q124R_Q160K_T178R | CAT-2200 | Pertuzumab |
| 12944-13471 | Q179K | K129T_S180E | L143E_K145T_S188L | Q124R_T178R | CAT-2200 | Pertuzumab |
| 13275-13470 | L143A_Q179K | K129T_V133W_S180E | L143E_K145T_S188L | Q124R_Q160K_T178R | CAT-2200 | Pertuzumab |
| 13276-13472 | L143A_Q179K | K129T_V133W_S180E | L143E_K145T_S188L | Q124R_T178R | CAT-2200 | Pertuzumab |
| 13308-13340 | Q179K | K129T_S180E | F122C_L143E_K145T | Q124C_Q160K_T178R | CAT-2200 | Pertuzumab |
| 12961-13093 | S186R | K129T_S180E | L143E_K145T_Q179E | Q124R_T178R | CAT-2200 | Pertuzumab |
| 12958-13079 | S186R | K129T_S180E | L143E_K145T_Q179E | Q124R_Q160K_T178R | CAT-2200 | Pertuzumab |
| 12938-13079 | Q179K | K129T_S180E | L143E_K145T_Q179E | Q124R_Q160K_T178R | CAT-2200 | Pertuzumab |
| 13323-13444 | S186R | K129T_S180E | L143E_K145T_Q179E | Q124R_T129K_T178R | CAT-2200 | Pertuzumab |
| 13310-13444 | Q179K | K129T_S180E | L143E_K145T_Q179E | Q124R_T129K_T178R | CAT-2200 | Pertuzumab |
| 13322-13443 | S186R | K129T_S180E | L143E_K145T_Q179E | Q124R_T129K_Q160K_T178R | CAT-2200 | Pertuzumab |
| 13316-13451 | Q179K | K129T_Y178E_S180E | L143E_K145T_Q179E | Q124R_T178R | CAT-2200 | Pertuzumab |
| 13314-13438 | Q179K | K129T_Y178E_S180E | L143E_K145T_Q179E | Q124R_Q160K_T178R | CAT-2200 | Pertuzumab |
| 12938-13106 | Q179K | K129T_S180E | L143E_K145T_Q179E_S188L | Q124R_Q160K_T178R | CAT-2200 | Pertuzumab |
| 12944-13108 | Q179K | K129T_S180E | L143E_K145T_Q179E_S188L | Q124R_T178R | CAT-2200 | Pertuzumab |
| 13275-13461 | L143A_Q179K | K129T_V133W_S180E | L143E_K145T_Q179E_S188L | Q124R_Q160K_T178R | CAT-2200 | Pertuzumab |
| 13276-13462 | L143A_Q179K | K129T_V133W_S180E | L143E_K145T_Q179E_S188L | Q124R_T178R | CAT-2200 | Pertuzumab |
| 13308-13341 | Q179K | K129T_S180E | F122C_L143E_K145T_Q179E | Q124C_Q160K_T178R | CAT-2200 | Pertuzumab |

TABLE 8A
LCCA results for K-L designs from the second K-L design
library that passed the performance cut off criteria

| | Column 2 | Column 3 | Column 4 | Column 5 | Column 6 | Column 7 | | |
|---|---|---|---|---|---|---|---|---|
| | H1L1:H1L2 | H1L1:H1L2 | H1L1:H1L2 | H2L2:H2L1 | H2L2:H2L1 | H1L2:H2L1 | | |
| Column 1 | median | range of | normalized | median | range of | noramlized | | |
| Unique | scalar | scalar | median | scalar | scalar | median | Column 8 | Column 9 |
| identifier | value | value | ratio | value | value | ratio | H1L1 Fab | H2L2 Fab |
| 12944-13093 | 1.41 | 1.04 | 79:21 | 1.39 | 0.27 | 80:20 | CAT-2200 | Pertuzumab |
| 12958-13064 | 1.29 | 1.13 | 77:23 | 0.86 | NA | 70:30 | CAT-2200 | Pertuzumab |
| 12958-13106 | 1.29 | 1.13 | 77:23 | 3.06 | 0.1 | 95:5 | CAT-2200 | Pertuzumab |
| 12958-13469 | 1.29 | 1.13 | 77:23 | 1.83 | 0.02 | 86:14 | CAT-2200 | Pertuzumab |
| 12961-13108 | 2 | NA | 88:12 | 1.82 | NA | 86:14 | CAT-2200 | Pertuzumab |
| 12961-13471 | 2 | NA | 88:12 | 1.16 | NA | 76:24 | CAT-2200 | Pertuzumab |
| 13200-13421 | −2.46 | NA | 7:93 | −0.26 | 0.35 | 44:56 | CAT-2200 | Pertuzumab |
| 13202-13426 | −2.18 | NA | 10:90 | −0.24 | NA | 44:56 | CAT-2200 | Pertuzumab |
| 13203-13427 | −2.68 | NA | 6:94 | 2.19 | 4.71 | 72:28 | CAT-2200 | Pertuzumab |
| 13248-13475 | 1.72 | NA | 84:16 | −0.23 | NA | 44:56 | CAT-2200 | Pertuzumab |
| 13309-13443 | 0.98 | NA | 72:28 | 1.35 | NA | 79:21 | CAT-2200 | Pertuzumab |
| 13322-13425 | 1.1 | 0.72 | 74:26 | −0.13 | 0.03 | 47:53 | CAT-2200 | Pertuzumab |
| 13204-13351 | 1.71 | 0.98 | 84:16 | −0.1 | 0.21 | 48:52 | CAT-2200 | Pertuzumab |
| 13205-13353 | 2.28 | NA | 90:10 | 0.07 | NA | 51:49 | CAT-2200 | Pertuzumab |
| 13196-13351 | 1 | NA | 73:27 | −0.1 | 0.21 | 48:52 | CAT-2200 | Pertuzumab |
| 13197-13353 | 0.37 | NA | 59:41 | 0.07 | NA | 51:49 | CAT-2200 | Pertuzumab |
| 13250-13355 | 1.38 | 0.6 | 79:21 | −0.45 | NA | 39:61 | CAT-2200 | Pertuzumab |
| 13277-13420 | 1.08 | 0.17 | 75:25 | 0.73 | 0.12 | 67:33 | CAT-2200 | Pertuzumab |
| 13319-13473 | −0.88 | 0.71 | 30:70 | 0.43 | 0.43 | 60:40 | CAT-2200 | Pertuzumab |
| 13188-13421 | 1.6 | 0.53 | 83:17 | −0.26 | 0.35 | 44:56 | CAT-2200 | Pertuzumab |
| 13190-13422 | 1.5 | NA | 81:19 | −0.32 | NA | 42:58 | CAT-2200 | Pertuzumab |
| 13242-13475 | 1.48 | NA | 81:19 | −0.23 | NA | 44:56 | CAT-2200 | Pertuzumab |
| 13192-13426 | 1.96 | NA | 87:13 | −0.24 | NA | 44:56 | CAT-2200 | Pertuzumab |
| 13194-13427 | 1.22 | 0.18 | 77:23 | 2.19 | 4.71 | 72:28 | CAT-2200 | Pertuzumab |
| 13317-13423 | −1.42 | NA | 19:81 | 3.09 | 1.62 | 94:6 | CAT-2200 | Pertuzumab |
| 13188-13430 | 1.6 | 0.53 | 83:17 | 0.77 | 0.25 | 68:32 | CAT-2200 | Pertuzumab |
| 13200-13430 | −2.46 | NA | 7:93 | 0.77 | 0.25 | 68:32 | CAT-2200 | Pertuzumab |
| 13246-13463 | 1.98 | 0.9 | 87:13 | −0.51 | 0.09 | 37:63 | CAT-2200 | Pertuzumab |
| 13238-13463 | 2.26 | 0.73 | 90:10 | −0.51 | 0.09 | 37:63 | CAT-2200 | Pertuzumab |
| 13248-13465 | 1.72 | NA | 84:16 | −0.29 | NA | 42:58 | CAT-2200 | Pertuzumab |
| 13242-13465 | 1.48 | NA | 81:19 | −0.29 | NA | 42:58 | CAT-2200 | Pertuzumab |
| 13246-13459 | 1.98 | 0.9 | 87:13 | −0.51 | NA | 37:63 | CAT-2200 | Pertuzumab |
| 13238-13459 | 2.26 | 0.73 | 90:10 | −0.51 | NA | 37:63 | CAT-2200 | Pertuzumab |
| 13192-13452 | 1.96 | NA | 87:13 | 1.15 | NA | 75:25 | CAT-2200 | Pertuzumab |
| 13194-13456 | 1.22 | 0.18 | 77:23 | 0.72 | 0.02 | 67:33 | CAT-2200 | Pertuzumab |
| 13202-13452 | −2.18 | NA | 10:90 | 1.15 | NA | 75:25 | CAT-2200 | Pertuzumab |
| 13203-13456 | −2.68 | NA | 6:94 | 0.72 | 0.02 | 67:33 | CAT-2200 | Pertuzumab |
| 13254-13508 | 1.68 | 0.94 | 83:17 | 0.8 | 0.08 | 69:31 | CAT-2200 | Pertuzumab |
| 13255-13509 | 1.67 | NA | 84:16 | 0 | NA | 49:51 | CAT-2200 | Pertuzumab |
| 13262-13481 | 2.41 | 0.41 | 91:9 | 1.01 | 0.15 | 73:27 | CAT-2200 | Pertuzumab |
| 13261-13480 | 1.91 | NA | 87:13 | 0.61 | NA | 64:36 | CAT-2200 | Pertuzumab |
| 13258-13504 | −0.25 | NA | 43:57 | 2.76 | NA | 94:6 | CAT-2200 | Pertuzumab |
| 13256-13502 | 0.53 | NA | 62:38 | 2.01 | NA | 88:12 | CAT-2200 | Pertuzumab |
| 13257-13503 | 0.85 | 0.63 | 70:30 | 2.48 | 0.41 | 92:8 | CAT-2200 | Pertuzumab |
| 13269-13403 | 2.58 | 1.35 | 92:8 | 0.35 | 0.23 | 58:42 | CAT-2200 | Pertuzumab |
| 13268-13401 | 2.25 | NA | 90:10 | 0.07 | NA | 51:49 | CAT-2200 | Pertuzumab |
| 12878-11240 | 1.85 | 0.37 | 86:14 | 2.2 | 0.04 | 90:10 | CAT-2200 | Pertuzumab |
| 10798-13036 | 2.29 | 0.65 | 90:10 | 2.34 | NA | 91:9 | CAT-2200 | Pertuzumab |
| 13265-13392 | 2.97 | 0.72 | 95:5 | −0.29 | NA | 42:58 | CAT-2200 | Pertuzumab |
| 13265-13419 | 2.97 | 0.72 | 95:5 | 0.54 | 0.12 | 63:37 | CAT-2200 | Pertuzumab |
| 13212-13396 | 3 | 0.15 | 95:5 | 0.29 | 0.2 | 57:43 | CAT-2200 | Pertuzumab |
| 13211-13395 | 2.12 | NA | 89:11 | 0.55 | NA | 63:37 | CAT-2200 | Pertuzumab |
| 13168-13397 | 3.03 | 0.12 | 95:5 | 1.97 | 5.15 | 67:33 | CAT-2200 | Pertuzumab |
| 13296-13399 | 1.05 | 0.04 | 74:26 | 0.55 | 0.45 | 63:37 | CAT-2200 | Pertuzumab |
| 13297-13402 | 1.21 | NA | 77:23 | 0.25 | NA | 56:44 | CAT-2200 | Pertuzumab |
| 13264-13400 | 1.14 | NA | 75:25 | 0.45 | NA | 61:39 | CAT-2200 | Pertuzumab |
| 13332-13360 | 1.69 | 0.38 | 84:16 | 2.25 | 0.47 | 90:10 | CAT-2200 | Pertuzumab |
| 13328-13359 | 2.12 | NA | 89:11 | 0.9 | NA | 71:29 | CAT-2200 | Pertuzumab |
| 13335-13361 | 2.01 | NA | 88:12 | 1.58 | NA | 82:18 | CAT-2200 | Pertuzumab |
| 13337-13362 | 2.14 | NA | 89:11 | 0.53 | NA | 63:37 | CAT-2200 | Pertuzumab |
| 13237-13358 | 2.33 | 0.56 | 91:9 | 0.59 | 0.22 | 64:36 | CAT-2200 | Pertuzumab |
| 13235-13357 | 1.96 | NA | 87:13 | −0.33 | NA | 41:59 | CAT-2200 | Pertuzumab |
| 13209-13364 | 2.54 | 0.19 | 92:8 | 1.21 | 0.05 | 77:23 | CAT-2200 | Pertuzumab |
| 13207-13363 | 2.09 | NA | 89:11 | 0.33 | NA | 58:42 | CAT-2200 | Pertuzumab |
| 13210-13510 | 1.5 | 0.14 | 81:19 | 2.55 | 0.51 | 92:8 | CAT-2200 | Pertuzumab |
| 13167-13514 | 2.61 | NA | 93:7 | 2.83 | 1.01 | 94:6 | CAT-2200 | Pertuzumab |
| 13294-13492 | 1.48 | 0.67 | 81:19 | 1.45 | 0.28 | 81:19 | CAT-2200 | Pertuzumab |
| 13292-13489 | 1.18 | 0.48 | 76:24 | 1.21 | 0.25 | 77:23 | CAT-2200 | Pertuzumab |
| 13263-13493 | 2 | 0.48 | 88:12 | 1.31 | 0.59 | 78:22 | CAT-2200 | Pertuzumab |
| 13259-13491 | 2.34 | NA | 91:9 | −0.02 | NA | 49:51 | CAT-2200 | Pertuzumab |
| 13212-13415 | 3 | 0.15 | 95:5 | 0.23 | 0.29 | 56:44 | CAT-2200 | Pertuzumab |
| 13169-13418 | 3.25 | 0.6 | 96:4 | 1.83 | 4.92 | 66:34 | CAT-2200 | Pertuzumab |
| 13331-13447 | 0.49 | NA | 61:39 | 3.52 | NA | 97:3 | CAT-2200 | Pertuzumab |
| 13327-13446 | 0.7 | NA | 66:34 | 3.01 | NA | 95:5 | CAT-2200 | Pertuzumab |
| 13334-13449 | 0.46 | NA | 61:39 | 2.74 | NA | 93:7 | CAT-2200 | Pertuzumab |
| 13336-13450 | 0.81 | 0.4 | 69:31 | 3.1 | 0.75 | 95:5 | CAT-2200 | Pertuzumab |
| 13236-13433 | 2.45 | 1.02 | 91:9 | 2.64 | NA | 93:7 | CAT-2200 | Pertuzumab |
| 13234-13432 | 2 | NA | 88:12 | 2.54 | NA | 92:8 | CAT-2200 | Pertuzumab |
| 13208-13455 | 1.54 | 0.48 | 82:18 | 3.5 | 1.89 | 95:5 | CAT-2200 | Pertuzumab |
| 13206-13454 | 1.2 | NA | 76:24 | 2.56 | NA | 92:8 | CAT-2200 | Pertuzumab |
| 13333-13387 | 1.64 | 0.38 | 83:17 | 4.11 | 1.06 | 98:2 | CAT-2200 | Pertuzumab |
| 13295-13484 | 1.44 | NA | 80:20 | 1.72 | 0.08 | 85:15 | CAT-2200 | Pertuzumab |
| 13282-13484 | 1.68 | 0.37 | 84:16 | 1.72 | 0.08 | 85:15 | CAT-2200 | Pertuzumab |
| 13218-13482 | 1.92 | 0.12 | 87:13 | 2.13 | 0.09 | 89:11 | CAT-2200 | Pertuzumab |
| 13219-13483 | 2.2 | NA | 89:11 | 0.87 | NA | 70:30 | CAT-2200 | Pertuzumab |
| 13175-13486 | 3.12 | 2.77 | 91:9 | 1.08 | 0.22 | 74:26 | CAT-2200 | Pertuzumab |
| 13176-13487 | 2.73 | NA | 93:7 | −0.2 | NA | 44:56 | CAT-2200 | Pertuzumab |
| 13280-13488 | 0.49 | NA | 62:38 | 0.02 | NA | 50:50 | CAT-2200 | Pertuzumab |
| 13281-13489 | 0.25 | NA | 56:44 | 1.21 | 0.25 | 77:23 | CAT-2200 | Pertuzumab |
| 13291-13488 | 1.41 | NA | 80:20 | 0.02 | NA | 50:50 | CAT-2200 | Pertuzumab |
| 13287-13494 | 1.73 | 0.28 | 85:15 | 1.25 | 0.13 | 77:23 | CAT-2200 | Pertuzumab |
| 13214-13405 | 2.08 | NA | 88:12 | −0.52 | NA | 37:63 | CAT-2200 | Pertuzumab |
| 13221-13406 | 2.38 | 0.29 | 91:9 | −0.41 | 0.26 | 40:60 | CAT-2200 | Pertuzumab |
| 13214-13410 | 2.08 | NA | 88:12 | 0 | NA | 49:51 | CAT-2200 | Pertuzumab |
| 13221-13411 | 2.38 | 0.29 | 91:9 | 0 | 0 | 50:50 | CAT-2200 | Pertuzumab |
| 13285-13414 | 1.63 | 0.18 | 83:17 | −0.44 | 0.1 | 39:61 | CAT-2200 | Pertuzumab |
| 13171-13416 | 3.11 | NA | 95:5 | −0.52 | NA | 37:63 | CAT-2200 | Pertuzumab |
| 13178-13417 | 2.74 | 1.26 | 93:7 | −0.49 | 0.1 | 38:62 | CAT-2200 | Pertuzumab |
| 13220-13511 | 1.56 | 1.08 | 81:19 | 1.72 | 0.22 | 85:15 | CAT-2200 | Pertuzumab |
| 13177-13513 | 1.58 | 0.73 | 82:18 | −0.08 | 0.07 | 47:53 | CAT-2200 | Pertuzumab |
| 13288-13516 | 1.41 | 0.68 | 80:20 | 0.88 | 0.26 | 70:30 | CAT-2200 | Pertuzumab |
| 13223-13512 | 0.95 | 1.17 | 70:30 | 2.03 | 0.22 | 88:12 | CAT-2200 | Pertuzumab |
| 13180-13515 | 1.56 | 0.75 | 82:18 | 1.74 | 0.42 | 85:15 | CAT-2200 | Pertuzumab |
| 13298-13439 | −1.21 | NA | 23:77 | 2.79 | NA | 94:6 | CAT-2200 | Pertuzumab |
| 13300-13441 | −0.72 | NA | 32:68 | 2.58 | 0 | 92:8 | CAT-2200 | Pertuzumab |
| 13271-13439 | −0.6 | NA | 35:65 | 2.79 | NA | 94:6 | CAT-2200 | Pertuzumab |
| 13273-13441 | −0.31 | 0.18 | 42:58 | 2.58 | 0 | 92:8 | CAT-2200 | Pertuzumab |
| 13299-13379 | −0.53 | NA | 37:63 | 3.26 | NA | 96:4 | CAT-2200 | Pertuzumab |
| 13301-13381 | −0.28 | 0.2 | 43:57 | 3.71 | 0.93 | 97:3 | CAT-2200 | Pertuzumab |
| 13272-13379 | −0.51 | NA | 37:63 | 3.26 | NA | 96:4 | CAT-2200 | Pertuzumab |
| 13274-13381 | −0.57 | NA | 36:64 | 3.71 | 0.93 | 97:3 | CAT-2200 | Pertuzumab |
| 13305-13448 | −1.5 | 0.06 | 18:82 | 3.63 | 1.65 | 96:4 | CAT-2200 | Pertuzumab |
| 13304-13466 | 0.63 | 0.22 | 65:35 | 0.99 | 0.24 | 73:27 | CAT-2200 | Pertuzumab |
| 13226-13434 | 1.82 | 0.08 | 86:14 | 2.68 | 0.36 | 93:7 | CAT-2200 | Pertuzumab |
| 13182-13458 | 1.53 | 0.76 | 81:19 | 1.1 | 2.96 | 66:34 | CAT-2200 | Pertuzumab |
| 13227-13383 | 2.47 | 1.62 | 90:10 | 2.31 | 0.51 | 90:10 | CAT-2200 | Pertuzumab |
| 13184-13388 | 1.4 | 0.79 | 79:21 | 3.08 | 0.13 | 95:5 | CAT-2200 | Pertuzumab |
| 13229-13428 | 1.44 | 1.12 | 79:21 | 2.02 | 0.28 | 88:12 | CAT-2200 | Pertuzumab |
| 13224-13428 | 1.48 | NA | 81:19 | 2.02 | 0.28 | 88:12 | CAT-2200 | Pertuzumab |
| 13231-13386 | 2.02 | 0.97 | 87:13 | 1.7 | 0.42 | 84:16 | CAT-2200 | Pertuzumab |
| 13199-13391 | 2.04 | NA | 88:12 | 1.9 | NA | 86:14 | CAT-2200 | Pertuzumab |
| 13230-13384 | 1.63 | NA | 83:17 | 1.71 | NA | 84:16 | CAT-2200 | Pertuzumab |
| 13198-13389 | 1.16 | 0.33 | 75:25 | 2.42 | 0.43 | 92:8 | CAT-2200 | Pertuzumab |
| 13225-13384 | 0.87 | NA | 70:30 | 1.71 | NA | 84:16 | CAT-2200 | Pertuzumab |
| 13181-13389 | 1.75 | NA | 85:15 | 2.42 | 0.43 | 92:8 | CAT-2200 | Pertuzumab |
| 13306-13375 | −1.45 | 0.02 | 19:81 | 0.85 | 0.33 | 70:30 | CAT-2200 | Pertuzumab |
| 13306-13373 | −1.45 | 0.02 | 19:81 | 0.48 | NA | 61:39 | CAT-2200 | Pertuzumab |
| 13185-13374 | −1.11 | 0.12 | 24:76 | 0.25 | NA | 56:44 | CAT-2200 | Pertuzumab |
| 13283-13507 | 1.09 | 0.31 | 74:26 | −0.45 | 0.26 | 39:61 | CAT-2200 | Pertuzumab |
| 13216-13505 | 1.39 | 1.28 | 78:22 | 0.96 | 0.05 | 72:28 | CAT-2200 | Pertuzumab |
| 13173-13506 | 1.33 | 0.47 | 78:22 | −0.5 | 0.02 | 37:63 | CAT-2200 | Pertuzumab |
| 13283-13478 | 1.09 | 0.31 | 74:26 | −0.49 | NA | 37:63 | CAT-2200 | Pertuzumab |
| 13216-13476 | 1.39 | 1.28 | 78:22 | 1.69 | NA | 84:16 | CAT-2200 | Pertuzumab |
| 13173-13477 | 1.33 | 0.47 | 78:22 | −0.18 | NA | 45:55 | CAT-2200 | Pertuzumab |
| 13329-13366 | 1.54 | 0.47 | 82:18 | 3.9 | 0.89 | 97:3 | CAT-2200 | Pertuzumab |
| 13252-13369 | 2.39 | 0.94 | 91:9 | 1.45 | 0.42 | 81:19 | CAT-2200 | Pertuzumab |
| 13289-13368 | 1.6 | 0.48 | 83:17 | 1.84 | 0.15 | 86:14 | CAT-2200 | Pertuzumab |
| 13338-13371 | 1.05 | 0.91 | 73:27 | 3.81 | 0.37 | 98:2 | CAT-2200 | Pertuzumab |
| 13278-13372 | 0.66 | 0.18 | 65:35 | 3.71 | 0.33 | 97:3 | CAT-2200 | Pertuzumab |
| 13320-13370 | 1.6 | 1.11 | 82:18 | 2.43 | 0.35 | 92:8 | CAT-2200 | Pertuzumab |
| 13302-13367 | 0.69 | 0.01 | 66:34 | 4.3 | 0.94 | 98:2 | CAT-2200 | Pertuzumab |
| 13330-13495 | 1.8 | 0.22 | 86:14 | 2.65 | 0.21 | 93:7 | CAT-2200 | Pertuzumab |
| 13253-13498 | 1.6 | 0.57 | 82:18 | 1.2 | 0.06 | 77:23 | CAT-2200 | Pertuzumab |
| 13290-13497 | 1.58 | 0.34 | 83:17 | 1.36 | 0.06 | 80:20 | CAT-2200 | Pertuzumab |
| 13339-13500 | 1.01 | 0.73 | 72:28 | 2.77 | 0.05 | 94:6 | CAT-2200 | Pertuzumab |
| 13279-13501 | 0.79 | 0.12 | 69:31 | 3.24 | 0.61 | 96:4 | CAT-2200 | Pertuzumab |
| 13321-13499 | 1.84 | 0.5 | 85:15 | 2.37 | 0.38 | 91:9 | CAT-2200 | Pertuzumab |
| 13303-13496 | 0.83 | NA | 69:31 | 3 | NA | 95:5 | CAT-2200 | Pertuzumab |
| 12957-13061 | 1.73 | 0.84 | 84:16 | 0.4 | 0.26 | 59:41 | CAT-2200 | Pertuzumab |
| 12906-13152 | 0.54 | 0.45 | 63:37 | 1.89 | 0.22 | 87:13 | CAT-2200 | Pertuzumab |
| 13318-13437 | 0.06 | 0.18 | 51:49 | 3.31 | 1.11 | 96:4 | CAT-2200 | Pertuzumab |

TABLE 8B
LCCA results for K-K-derived K-L designs from the second K-K derived K-L
design library that passed the performance cut-off criteria
LCCA results for K-K-derived K-L designs from the second K-K derived
design library that passed the performance cut-off criteria

| | Column 2 | Column 3 | Column 4 | Column 5 | Column 6 | Column 7 | | |
|---|---|---|---|---|---|---|---|---|
| Column 1 | H1L1:H1L2 | H1L1:H1L2 | H1L1:H1L2 | H2L2:H2L1 | H2L2:H2L1 | H1L2:H2L1 | | |
| Unique | median | range of | normalized | median | range of | normalized | Column 8 | Column 9 |
| identifier | scalar value | scalar value | median ratio | scalar value | scalar value | median ratio | H1L1 Fab | H2L2 Fab |
| 12938-13064 | 1.96 | 0.19 | 87:13 | 0.86 | NA | 70:30 | CAT-2200 | Pertuzumab |
| 13309-13425 | 0.98 | NA | 72:28 | −0.13 | 0.03 | 47:53 | CAT-2200 | Pertuzumab |
| 13314-13424 | 0.89 | 0.3 | 70:30 | 2.02 | 1.32 | 86:14 | CAT-2200 | Pertuzumab |
| 12938-13469 | 1.96 | 0.19 | 87:13 | 1.83 | 0.02 | 86:14 | CAT-2200 | Pertuzumab |
| 12944-13471 | 1.41 | 1.04 | 79:21 | 1.16 | NA | 76:24 | CAT-2200 | Pertuzumab |
| 13275-13470 | 1.51 | 1.16 | 80:20 | 1.94 | NA | 87:13 | CAT-2200 | Pertuzumab |
| 13276-13472 | 0.88 | 0.11 | 70:30 | 2.16 | 0.12 | 90:10 | CAT-2200 | Pertuzumab |
| 13308-13340 | 0.98 | 0.32 | 72:28 | 2.34 | 0.8 | 90:10 | CAT-2200 | Pertuzumab |
| 12961-13093 | 2 | NA | 88:12 | 1.39 | 0.27 | 80:20 | CAT-2200 | Pertuzumab |
| 12958-13079 | 1.29 | 1.13 | 77:23 | 1.87 | NA | 86:14 | CAT-2200 | Pertuzumab |
| 12938-13079 | 1.96 | 0.19 | 87:13 | 1.87 | NA | 86:14 | CAT-2200 | Pertuzumab |
| 13323-13444 | 1.31 | NA | 78:22 | 1.29 | NA | 78:22 | CAT-2200 | Pertuzumab |
| 13310-13444 | 1.37 | NA | 79:21 | 1.29 | NA | 78:22 | CAT-2200 | Pertuzumab |
| 13322-13443 | 1.1 | 0.72 | 74:26 | 1.35 | NA | 79:21 | CAT-2200 | Pertuzumab |
| 13316-13451 | 1.22 | 0.26 | 77:23 | 3.71 | 1.94 | 96:4 | CAT-2200 | Pertuzumab |
| 13314-13438 | 0.89 | 0.3 | 70:30 | 2.81 | NA | 94:6 | CAT-2200 | Pertuzumab |
| 12938-13106 | 1.96 | 0.19 | 87:13 | 3.06 | 0.1 | 95:5 | CAT-2200 | Pertuzumab |
| 12944-13108 | 1.41 | 1.04 | 79:21 | 1.82 | NA | 86:14 | CAT-2200 | Pertuzumab |
| 13275-13461 | 1.51 | 1.16 | 80:20 | 2.23 | 0.45 | 90:10 | CAT-2200 | Pertuzumab |
| 13276-13462 | 0.88 | 0.11 | 70:30 | 2.29 | NA | 90:10 | CAT-2200 | Pertuzumab |
| 13308-13341 | 0.98 | 0.32 | 72:28 | 2.04 | 0.71 | 88:12 | CAT-2200 | Pertuzumab |

TABLE 9A
Antigen binding and thermal stability assessment of the K-L designs that passed the LCCA performance cut-off criteria

| | | | | | | | | Column | | Column | | Column |
|---|---|---|---|---|---|---|---|---|---|---|---|---|
| | | | | | | | | 9 | | 11 | | 13 |
| | | | | | | Column 7 | Column | Change | Column | Change | Column | Change |
| | | Column 3 | | Column 5 | | Change | 8 | in DSF | 10 | in DSC | 12 | in DSC |
| | | Change in | | Change in | Column 6 | in DSF | DSF | values | DSC | values | DSC | values |
| | Column 2 | KD of | Column 4 | KD of | DSF | values of | values | of | values | of | values | of |
| | KD of | H1L1 | KD of | H2L2 | values of | H1L1 | of | H2L2 | of | H1L1 | of | H2L2 |
| Column 1 | H1L1 | Fab vs | H2L2 | Fab vs | H1L1 | Fab vs | H2L2 | Fab vs | H1L1 | Fab vs | H2L2 | Fab vs |
| Unique | Fab | wild | Fab | wild | Fab | wild | Fab | wild | Fab | wild | Fab | wild |
| identifier | (nM) | type | (nM) | type | (° C.) | type | (° C.) | type | (° C.) | type | (° C.) | type |
| 10798-13036 | 0.34 | −0.22 | ND | ND | 74 | −3 | ND | ND | 69.7 | −1 | ND | ND |
| 11240-12878 | 0.34 | −0.22 | 7.9 | −0.0068 | 74 | −3 | 80.8 | −0.9 | 69.7 | −1 | 78.2 | 0.1 |
| 12906-13152 | 0.33 | −0.19 | 7 | 0.044 | 74 | −3 | 78.5 | −3.2 | ND | ND | ND | ND |
| 12944-13093 | 0.24 | −0.056 | 7 | 0.046 | 78 | 1 | 81.3 | −0.4 | #70.7 | 0 | 77.9 | −0.2 |
| 12957-13061 | 0.26 | −0.088 | 7.4 | 0.02 | 75.2 | −1.8 | 81.7 | 0 | 69.6 | −1.1 | ND | ND |
| 12958-13064 | 0.26 | −0.088 | 7.9 | −0.007 | 75.2 | −1.8 | 81.3 | −0.4 | 69.6 | −1.1 | 77.8 | −0.3 |
| 12958-13106 | 0.26 | −0.088 | 6.9 | 0.051 | 75.2 | −1.8 | 81 | −0.7 | 69.6 | −1.1 | 78.2 | 0.1 |
| 12958-13469 | 0.26 | −0.088 | 7.7 | 0.0027 | 75.2 | −1.8 | 80 | −1.7 | 69.6 | −1.1 | 77.2 | −0.9 |
| 12961-13108 | 0.26 | −0.088 | 7.4 | 0.02 | 75.2 | −1.8 | 81.5 | −0.2 | 69.6 | −1.1 | ND | ND |
| 12961-13471 | 0.26 | −0.088 | 8 | −0.012 | 75.2 | −1.8 | 81.3 | −0.4 | 69.6 | −1.1 | 77.9 | −0.2 |
| 13167-13514* | 0.19 | 0.037 | 8.3 | −0.029 | 74.5 | −2.5 | 80 | −1.7 | 70 | −0.7 | 77.2 | −0.9 |
| 13168-13397^{‡} | 0.19 | 0.037 | 7.8 | 4.80E−04 | 74.5 | −2.5 | 76.8 | −4.9 | 70 | −0.7 | ND | ND |
| 13169-13418* | 0.19 | 0.037 | 7.6 | 0.0089 | 74.5 | −2.5 | 78.3 | −3.4 | 70 | −0.7 | ND | ND |
| 13171-13416 | ND | ND | 7.6 | 0.0089 | ND | ND | 78.3 | −3.4 | ND | ND | ND | ND |
| 13173-13477* | 0.088 | 0.38 | ND | ND | 75.3 | −1.7 | ND | ND | ND | ND | ND | ND |
| 13173-13506^{‡} | 0.088 | 0.38 | 9.7 | −0.096 | 75.3 | −1.7 | 72.7 | −9 | ND | ND | ND | ND |
| 13175-13486^{‡} | 0.16 | 0.11 | 9.1 | −0.07 | 74.8 | −2.2 | 77.5 | −4.2 | 70 | −0.7 | 74.7 | −3.4 |
| 13176-13487* | 0.16 | 0.11 | ND | ND | 74.8 | −2.2 | ND | ND | 70 | −0.7 | ND | ND |
| 13177-13513^{‡} | 0.16 | 0.11 | 9.7 | −0.096 | 74.8 | −2.2 | 76.8 | −4.9 | 70 | −0.7 | 73.6 | −4.5 |
| 13178-13417* | 0.16 | 0.11 | 7.6 | 0.0089 | 74.8 | −2.2 | 78.3 | −3.4 | 70 | −0.7 | ND | ND |
| 13180-13515^{†} | 0.028 | 0.88 | 7.5 | 0.015 | 73.3 | −3.7 | 75.5 | −6.2 | ND | ND | ND | ND |
| 13181-13389 | ND | ND | 7.6 | 0.0078 | ND | ND | 79.8 | −1.9 | ND | ND | 76.3 | −1.8 |
| 13182-13458^{‡} | 0.11 | 0.3 | 7.5 | 0.014 | 74 | −3 | 79.5 | −2.2 | 69.5 | −1.2 | 76.2 | −1.9 |
| 13184-13388* | 0.11 | 0.3 | 7.6 | 0.0078 | 74 | −3 | 79.8 | −1.9 | 69.5 | −1.2 | 76.3 | −1.8 |
| 13185-13374 | ND | ND | 9.2 | −0.07 | ND | ND | ND | ND | ND | ND | ND | ND |
| 13188-13421 | 0.22 | −0.028 | 7.4 | 0.024 | 77.5 | 0.5 | 77 | −4.7 | #70.7 | 0 | ND | ND |
| 13188-13430 | 0.22 | −0.028 | 8 | −0.011 | 77.5 | 0.5 | 78.8 | −2.9 | #70.7 | 0 | 76.1 | −2 |
| 13190-13422 | 0.22 | −0.028 | 7.7 | 0.005 | 77.5 | 0.5 | 78.5 | −3.2 | #70.7 | 0 | 75.8 | −2.3 |
| 13192-13426 | 0.22 | −0.028 | ND | ND | 77.5 | 0.5 | ND | ND | #70.7 | 0 | ND | ND |
| 13192-13452 | 0.22 | −0.028 | ND | ND | 77.5 | 0.5 | ND | ND | #70.7 | 0 | ND | ND |
| 13194-13427^{†} | 0.22 | −0.028 | 7.6 | 0.012 | 77.5 | 0.5 | 79.3 | −2.4 | #70.7 | 0 | 76.5 | −1.6 |
| 13194-13456^{†} | 0.22 | −0.028 | 7.5 | 0.014 | 77.5 | 0.5 | 79.5 | −2.2 | #70.7 | 0 | 76.2 | −1.9 |
| 13196-13351^{†} | 0.22 | −0.028 | 7.1 | 0.043 | 77.5 | 0.5 | 80 | −1.7 | #70.7 | 0 | 76.8 | −1.3 |
| 13197-13353 | 0.22 | −0.028 | ND | ND | 77.5 | 0.5 | ND | ND | #70.7 | 0 | ND | ND |
| 13198-13389* | 0.13 | 0.2 | 7.6 | 0.0078 | 73.3 | −3.7 | 79.8 | −1.9 | 69.2 | −1.5 | 76.3 | −1.8 |
| 13199-13391 | ND | ND | 7.6 | 0.0078 | ND | ND | 79.8 | −1.9 | ND | ND | 76.3 | −1.8 |
| 13200-13421 | 0.21 | −0.0041 | 7.4 | 0.024 | 75 | −2 | 77 | −4.7 | ND | ND | ND | ND |
| 13200-13430 | 0.21 | −0.0041 | 8 | −0.011 | 75 | −2 | 78.8 | −2.9 | ND | ND | 76.1 | −2 |
| 13202-13426 | 0.21 | −0.0041 | ND | ND | 75 | −2 | ND | ND | ND | ND | ND | ND |
| 13202-13452 | 0.21 | −0.0041 | ND | ND | 75 | −2 | ND | ND | ND | ND | ND | ND |
| 13203-13427^{†} | 0.21 | −0.0041 | 7.6 | 0.012 | 75 | −2 | 79.3 | −2.4 | ND | ND | 76.5 | −1.6 |
| 13203-13456^{†} | 0.21 | −0.0041 | 7.5 | 0.014 | 75 | −2 | 79.5 | −2.2 | ND | ND | 76.2 | −1.9 |
| 13204-13351^{†} | 0.21 | −0.0041 | 7.1 | 0.043 | 75 | −2 | 80 | −1.7 | ND | ND | 76.8 | −1.3 |
| 13205-13353 | 0.21 | −0.0041 | ND | ND | 75 | −2 | ND | ND | ND | ND | ND | ND |
| 13206-13454^{†} | ND | ND | 7.5 | 0.014 | ND | ND | 79.5 | −2.2 | ND | ND | 76.2 | −1.9 |
| 13207-13363 | ND | ND | 7.9 | −0.0084 | ND | ND | 79 | −2.7 | ND | ND | 76.1 | −2 |
| 13208-13455^{‡} | 0.19 | 0.046 | 7.5 | 0.014 | 72.7 | −4.3 | 79.5 | −2.2 | 68.8 | −1.9 | 76.2 | −1.9 |
| 13209-13364* | 0.19 | 0.046 | 7.9 | −0.0084 | 72.7 | −4.3 | 79 | −2.7 | 68.8 | −1.9 | 76.1 | −2 |
| 13210-13510* | 0.18 | 0.067 | 7.5 | 0.015 | 74.2 | −2.8 | 80.5 | −1.2 | 69.8 | −0.9 | 77 | −1.1 |
| 13211-13395* | 0.18 | 0.067 | ND | ND | 74.2 | −2.8 | ND | ND | 69.8 | −0.9 | ND | ND |
| 13212-13396* | 0.18 | 0.067 | 8.1 | −0.017 | 74.2 | −2.8 | 78 | −3.7 | 69.8 | −0.9 | 75.3 | −2.8 |
| 13212-13415* | 0.18 | 0.067 | 8.8 | −0.053 | 74.2 | −2.8 | 78.3 | −3.4 | 69.8 | −0.9 | 74.6 | −3.5 |
| 13214-13405* | 0.17 | 0.095 | 8.1 | −0.018 | 74 | −3 | 78 | −3.7 | ND | ND | ND | ND |
| 13214-13410* | 0.17 | 0.095 | 8.4 | −0.034 | 74 | −3 | 77.5 | −4.2 | ND | ND | ND | ND |
| 13216-13476* | 0.078 | 0.43 | 7.6 | 0.01 | 73.7 | −3.3 | 74.5 | −7.2 | 69.9 | −0.8 | 71.2 | −6.9 |
| 13216-13505* | 0.078 | 0.43 | 9.6 | −0.093 | 73.7 | −3.3 | 75 | −6.7 | 69.9 | −0.8 | ND | ND |
| 13218-13482* | 0.19 | 0.044 | 7.6 | 0.01 | 74.5 | −2.5 | 77.8 | −3.9 | 70.3 | −0.4 | 75.2 | −2.9 |
| 13219-13483* | 0.19 | 0.044 | ND | ND | 74.5 | −2.5 | ND | ND | 70.3 | −0.4 | ND | ND |
| 13220-13511* | 0.19 | 0.044 | 7 | 0.044 | 74.5 | −2.5 | 76.7 | −5 | 70.3 | −0.4 | ND | ND |
| 13221-13406* | 0.19 | 0.044 | 8.1 | −0.018 | 74.5 | −2.5 | 78 | −3.7 | 70.3 | −0.4 | ND | ND |
| 13221-13411* | 0.19 | 0.044 | 8.4 | −0.034 | 74.5 | −2.5 | 77.5 | −4.2 | 70.3 | −0.4 | ND | ND |
| 13223-13512 | 0.12 | 0.25 | 7.3 | 0.028 | 73 | −4 | 76.8 | −4.9 | ND | ND | ND | ND |
| 13224-13428 | ND | ND | 8 | −0.011 | ND | ND | 78.8 | −2.9 | ND | ND | 76.1 | −2 |
| 13225-13384 | ND | ND | 7.4 | 0.02 | ND | ND | 77.5 | −4.2 | ND | ND | 75.3 | −2.8 |
| 13226-13434* | 0.15 | 0.15 | 8 | −0.011 | 73.5 | −3.5 | 79 | −2.7 | 69.5 | −1.2 | 76.1 | −2 |
| 13227-13383* | 0.15 | 0.15 | 7.4 | 0.02 | 73.5 | −3.5 | 77.5 | −4.2 | 69.5 | −1.2 | 75.3 | −2.8 |
| 13229-13428 | 0.13 | 0.2 | 8 | −0.011 | 72.3 | −4.7 | 78.8 | −2.9 | ND | ND | 76.1 | −2 |
| 13230-13384 | 0.13 | 0.2 | 7.4 | 0.02 | 72.3 | −4.7 | 77.5 | −4.2 | ND | ND | 75.3 | −2.8 |
| 13231-13386* | 0.12 | 0.24 | 7.4 | 0.02 | 72 | −5 | 77.5 | −4.2 | 68.2 | −2.5 | 75.3 | −2.8 |
| 13234-13432* | 0.16 | 0.1 | 8 | −0.011 | 73 | −4 | 79 | −2.7 | ND | ND | 76.1 | −2 |
| 13235-13357^{‡} | 0.16 | 0.1 | 7.3 | 0.029 | 73 | −4 | 78.5 | −3.2 | ND | ND | 75.1 | −3 |
| 13236-13433* | 0.13 | 0.19 | 8 | −0.011 | 72.5 | −4.5 | 79 | −2.7 | 69.1 | −1.6 | 76.1 | −2 |
| 13237-13358^{‡} | 0.13 | 0.19 | 7.3 | 0.029 | 72.5 | −4.5 | 78.5 | −3.2 | 69.1 | −1.6 | 75.1 | −3 |
| 13238-13459 | 0.36 | −0.24 | ND | ND | 77 | 0 | ND | ND | #70.1 | −0.6 | ND | ND |
| 13238-13463^{†} | 0.36 | −0.24 | 7.8 | −8.10E−05 | 77 | 0 | 78.8 | −2.9 | #70.1 | −0.6 | ND | ND |
| 13242-13465 | 0.36 | −0.24 | ND | ND | 77 | 0 | ND | ND | #70.1 | −0.6 | ND | ND |
| 13242-13475 | 0.36 | −0.24 | ND | ND | 77 | 0 | ND | ND | #70.1 | −0.6 | ND | ND |
| 13246-13459 | 0.32 | −0.19 | ND | ND | 75.5 | −1.5 | ND | ND | ND | ND | ND | ND |
| 13246-13463^{†} | 0.32 | −0.19 | 7.8 | −8.10E−05 | 75.5 | −1.5 | 78.8 | −2.9 | ND | ND | ND | ND |
| 13248-13465 | 0.32 | −0.19 | ND | ND | 75.5 | −1.5 | ND | ND | ND | ND | ND | ND |
| 13248-13475 | 0.32 | −0.19 | ND | ND | 75.5 | −1.5 | ND | ND | ND | ND | ND | ND |
| 13250-13355 | 0.32 | −0.19 | ND | ND | 75.5 | −1.5 | ND | ND | ND | ND | ND | ND |
| 13252-13369 | 0.34 | −0.22 | 6.7 | 0.065 | 74 | −3 | 81 | −0.7 | 69.7 | −1 | 77.4 | −0.7 |
| 13253-13498 | 0.34 | −0.22 | 7.5 | 0.016 | 74 | −3 | 79.5 | −2.2 | 69.7 | −1 | 76.3 | −1.8 |
| 13254-13508 | 0.34 | −0.22 | 8.2 | −0.022 | 74 | −3 | 74.5 | −7.2 | 69.7 | −1 | ND | ND |
| 13255-13509 | 0.34 | −0.22 | ND | ND | 74 | −3 | ND | ND | 69.7 | −1 | ND | ND |
| 13256-13502 | 0.34 | −0.22 | ND | ND | 74 | −3 | ND | ND | 69.7 | −1 | ND | ND |
| 13257-13503 | 0.34 | −0.22 | 8.4 | −0.035 | 74 | −3 | 81.3 | −0.4 | 69.7 | −1 | 78.4 | 0.3 |
| 13258-13504 | 0.34 | −0.22 | 7.2 | 0.032 | 74 | −3 | 82 | 0.3 | 69.7 | −1 | ND | ND |
| 13259-13491 | 0.34 | −0.22 | 7.9 | −0.0078 | 74 | −3 | 77.3 | −4.4 | 69.7 | −1 | ND | ND |
| 13261-13480 | 0.34 | −0.22 | ND | ND | 74 | −3 | ND | ND | 69.7 | −1 | ND | ND |
| 13262-13481 | 0.34 | −0.22 | 7.2 | 0.034 | 74 | −3 | 75.7 | −6 | 69.7 | −1 | 73.2 | −4.9 |
| 13263-13493 | 0.34 | −0.22 | 8.1 | −0.015 | 74 | −3 | 79.7 | −2 | 69.7 | −1 | 76.2 | −1.9 |
| 13264-13400 | 0.34 | −0.22 | 8.2 | −0.024 | 74 | −3 | 79.3 | −2.4 | 69.7 | −1 | ND | ND |
| 13265-13392 | 0.34 | −0.22 | ND | ND | 74 | −3 | ND | ND | 69.7 | −1 | ND | ND |
| 13265-13419 | 0.34 | −0.22 | 7.6 | 0.011 | 74 | −3 | 76.5 | −5.2 | 69.7 | −1 | 75.3 | −2.8 |
| 13268-13401 | 0.34 | −0.22 | ND | ND | 74 | −3 | ND | ND | 69.7 | −1 | ND | ND |
| 13269-13403 | 0.34 | −0.22 | 7.8 | 0.0016 | 74 | −3 | 79.8 | −1.9 | 69.7 | −1 | 76.1 | −2 |
| 13271-13439 | ND | ND | 7.6 | 0.012 | ND | ND | 80.8 | −0.9 | ND | ND | 77.4 | −0.7 |
| 13272-13379 | ND | ND | 8.1 | −0.016 | ND | ND | 80 | −1.7 | ND | ND | 76.5 | −1.6 |
| 13273-13441 | 0.099 | 0.32 | 7.6 | 0.012 | 74.5 | −2.5 | 80.8 | −0.9 | 70.6 | −0.1 | 77.4 | −0.7 |
| 13274-13381 | 0.099 | 0.32 | 8.1 | −0.016 | 74.5 | −2.5 | 80 | −1.7 | 70.6 | −0.1 | 76.5 | −1.6 |
| 13277-13420* | 0.17 | 0.085 | 7.3 | 0.028 | 73 | −4 | 78.2 | −3.5 | ND | ND | ND | ND |
| 13278-13372 | 0.35 | −0.22 | 5.9 | 0.12 | 74.2 | −2.8 | 80.5 | −1.2 | ND | ND | ND | ND |
| 13279-13501 | 0.35 | −0.22 | 7.5 | 0.015 | 74.2 | −2.8 | 79.3 | −2.4 | ND | ND | ND | ND |
| 13280-13488 | 0.35 | −0.22 | ND | ND | 74.2 | −2.8 | ND | ND | ND | ND | ND | ND |
| 13281-13489 | 0.35 | −0.22 | 7.9 | −0.0078 | 74.2 | −2.8 | 77.3 | −4.4 | ND | ND | ND | ND |
| 13282-13484 | 0.35 | −0.22 | 7.9 | −0.0045 | 74.2 | −2.8 | 77.3 | −4.4 | ND | ND | 74.6 | −3.5 |
| 13283-13478* | 0.098 | 0.33 | ND | ND | 73.5 | −3.5 | ND | ND | ND | ND | ND | ND |
| 13283-13507* | 0.098 | 0.33 | 7.3 | 0.026 | 73.5 | −3.5 | 75.3 | −6.4 | ND | ND | ND | ND |
| 13285-13414^{†} | 0.16 | 0.12 | 7.2 | 0.035 | 75.5 | −1.5 | 77.8 | −3.9 | 69.7 | −1 | 74.5 | −3.6 |
| 13287-13494 | 0.15 | 0.15 | 7.6 | 0.012 | 76.3 | −0.7 | 78.5 | −3.2 | #69.6 | −1.1 | 75.4 | −2.7 |
| 13288-13516^{†} | 0.16 | 0.11 | 7.3 | 0.026 | 73.7 | −3.3 | 77.8 | −3.9 | 69 | −1.7 | 74.6 | −3.5 |
| 13289-13368 | 0.29 | −0.14 | 6.7 | 0.065 | 73 | −4 | 81 | −0.7 | 69.1 | −1.6 | 77.4 | −0.7 |
| 13290-13497 | 0.29 | −0.14 | 7.5 | 0.016 | 73 | −4 | 79.5 | −2.2 | 69.1 | −1.6 | 76.3 | −1.8 |
| 13291-13488 | 0.29 | −0.14 | ND | ND | 73 | −4 | ND | ND | 69.1 | −1.6 | ND | ND |
| 13292-13489 | 0.29 | −0.14 | 7.9 | −0.0078 | 73 | −4 | 77.3 | −4.4 | 69.1 | −1.6 | ND | ND |
| 13294-13492 | 0.29 | −0.14 | 8.1 | −0.015 | 73 | −4 | 79.7 | −2 | 69.1 | −1.6 | 76.2 | −1.9 |
| 13295-13484 | 0.29 | −0.14 | 7.9 | −0.0045 | 73 | −4 | 77.3 | −4.4 | 69.1 | −1.6 | 74.6 | −3.5 |
| 13296-13399 | 0.29 | −0.14 | 8.2 | −0.024 | 73 | −4 | 79.3 | −2.4 | 69.1 | −1.6 | ND | ND |
| 13297-13402 | 0.29 | −0.14 | 7.8 | 0.0016 | 73 | −4 | 79.8 | −1.9 | 69.1 | −1.6 | 76.1 | −2 |
| 13298-13439 | 0.12 | 0.26 | 7.6 | 0.012 | 72.2 | −4.8 | 80.8 | −0.9 | ND | ND | 77.4 | −0.7 |
| 13299-13379 | 0.12 | 0.26 | 8.1 | −0.016 | 72.2 | −4.8 | 80 | −1.7 | ND | ND | 76.5 | −1.6 |
| 13300-13441 | 0.11 | 0.29 | 7.6 | 0.012 | 72.8 | −4.2 | 80.8 | −0.9 | ND | ND | 77.4 | −0.7 |
| 13301-13381 | 0.11 | 0.29 | 8.1 | −0.016 | 72.8 | −4.2 | 80 | −1.7 | ND | ND | 76.5 | −1.6 |
| 13302-13367 | 0.26 | −0.09 | 5.8 | 0.13 | 73.8 | −3.2 | 80.7 | −1 | 69.5 | −1.2 | 77.3 | −0.8 |
| 13303-13496 | 0.26 | −0.09 | 7.9 | −0.0084 | 73.8 | −3.2 | 79.5 | −2.2 | 69.5 | −1.2 | ND | ND |
| 13304-13466^{‡} | 0.15 | 0.14 | 8.2 | −0.024 | 72.5 | −4.5 | 79.7 | −2 | 68.3 | −2.4 | 76.5 | −1.6 |
| 13305-13448* | 0.097 | 0.33 | 7 | 0.046 | ND | ND | 81.3 | −0.4 | ND | ND | 77.9 | −0.2 |
| 13306-13373* | 0.097 | 0.33 | ND | ND | ND | ND | ND | ND | ND | ND | ND | ND |
| 13306-13375^{‡} | 0.097 | 0.33 | 8 | −0.012 | ND | ND | 64.7 | −17 | ND | ND | ND | ND |
| 13309-13443 | 0.24 | −0.056 | ND | ND | 78 | 1 | ND | ND | #70.7 | 0 | ND | ND |
| 13317-13423 | 0.12 | 0.23 | 7.9 | −0.007 | 73.3 | −3.7 | 81.3 | −0.4 | ND | ND | 77.8 | −0.3 |
| 13318-13437* | 0.1 | 0.3 | 7.2 | 0.035 | 72.5 | −4.5 | 81.2 | −0.5 | ND | ND | ND | ND |
| 13319-13473* | 0.18 | 0.072 | 8.1 | −0.02 | 73.5 | −3.5 | 80.7 | −1 | 69.5 | −1.2 | 78.3 | 0.2 |
| 13320-13370 | 0.26 | −0.088 | 6 | 0.11 | 75.2 | −1.8 | 80.5 | −1.2 | 69.6 | −1.1 | 76.9 | −1.2 |
| 13321-13499 | 0.26 | −0.088 | 7.4 | 0.023 | 75.2 | −1.8 | 79 | −2.7 | 69.6 | −1.1 | 76 | −2.1 |
| 13322-13425 | 0.26 | −0.088 | ND | ND | 75.2 | −1.8 | ND | ND | 69.6 | −1.1 | ND | ND |
| 13327-13446 | ND | ND | 7 | 0.046 | ND | ND | 81.3 | −0.4 | ND | ND | 77.9 | −0.2 |
| 13328-13359 | ND | ND | 7.5 | 0.019 | ND | ND | 80.2 | −1.5 | ND | ND | 77.3 | −0.8 |
| 13329-13366* | 0.23 | −0.049 | 5.8 | 0.13 | 72.3 | −4.7 | 80.7 | −1 | ND | ND | 77.3 | −0.8 |
| 13330-13495* | 0.23 | −0.049 | 7.9 | −0.0084 | 72.3 | −4.7 | 79.5 | −2.2 | ND | ND | ND | ND |
| 13331-13447* | 0.23 | −0.049 | 7 | 0.046 | 72.3 | −4.7 | 81.3 | −0.4 | ND | ND | 77.9 | −0.2 |
| 13332-13360* | 0.23 | −0.049 | 7.5 | 0.019 | 72.3 | −4.7 | 80.2 | −1.5 | ND | ND | 77.3 | −0.8 |
| 13333-13387* | 0.23 | −0.049 | 8.1 | −0.018 | 72.3 | −4.7 | 80.8 | −0.9 | ND | ND | 77.5 | −0.6 |
| 13334-13449* | 0.14 | 0.16 | 7 | 0.046 | 73.5 | −3.5 | 81.3 | −0.4 | 69.7 | −1 | 77.9 | −0.2 |
| 13335-13361* | 0.14 | 0.16 | 7.5 | 0.019 | 73.5 | −3.5 | 80.2 | −1.5 | 69.7 | −1 | 77.3 | −0.8 |
| 13336-13450* | 0.12 | 0.26 | 7 | 0.046 | 72.7 | −4.3 | 81.3 | −0.4 | ND | ND | 77.9 | −0.2 |
| 13337-13362* | 0.12 | 0.26 | 7.5 | 0.019 | 72.7 | −4.3 | 80.2 | −1.5 | ND | ND | 77.3 | −0.8 |
| 13338-13371 | 0.36 | −0.24 | 5.9 | 0.12 | 72.7 | −4.3 | 81.5 | −0.2 | 68.8 | −1.9 | ND | ND |
| 13339-13500 | 0.36 | −0.24 | 7.4 | 0.023 | 72.7 | −4.3 | 80 | −1.7 | 68.8 | −1.9 | ND | ND |

H1L1 Fab is CAT-2200 and the H2L2 Fab is Pertuzumab.
A number of heterodimers had precipitate associated with them, noted with
*in Tables 9A and 9B if the precipitate was associated with the H1L1 Fab; noted with
^{†}in Tables 9A and 9B is the precipitate was associated with H2L2 Fab; and noted with
^{‡}in Tables 9A and 9B if the precipitate was associated with both the H1L1 and H2L2 Fabs.
#denotes the presence of a shoulder to the main Fab peak (transition) in DSC thermogram

TABLE 9B
Antigen binding and thermal stablity assessment of the K-K derived K-L designs that passed the LCCA performance cut-off criteria

| | | Column | | Column | | Column | Column | Column | | Column | Column | Column |
|---|---|---|---|---|---|---|---|---|---|---|---|---|
| | | 3 | | 5 | Column | 7 | 8 | 9 | Column | 11 | 12 | 13 |
| | Column | Change | Column | Change | 6 | Change | DSF | Change | 10 | Change | DSC | Change |
| | 2 | in KD | 4 | in KD of | DSF | in DSF | values | in DSF | DSC | in DSC | values | in DSC |
| | KD of | of H1L1 | KD of | H2L2 | values of | values | of | values | values | values | of | values |
| Column 1 | H1L1 | Fab | H2L2 | Fab | H1L1 | of H1L1 | H2L2 | of H2L2 | of H1L1 | of H1L1 | H2L2 | of H2L2 |
| Unique | Fab | vs wild | Fab | vs wild | Fab | Fab vs | Fab | Fab vs | Fab | Fab vs | Fab | Fab vs |
| identifier | (nM) | type | (nM) | type | (° C.) | wild type | (° C.) | wild type | (° C.) | wild type | (° C.) | wild type |
| 12938-13064 | 0.24 | −0.056 | 7.9 | −0.007 | 78 | 1 | 81.3 | −0.4 | #70.7 | 0 | 77.8 | −0.3 |
| 12938-13079 | 0.24 | −0.056 | 7.2 | 0.035 | 78 | 1 | 81.2 | −0.5 | #70.7 | 0 | ND | ND |
| 12938-13106 | 0.24 | −0.056 | 6.9 | 0.051 | 78 | 1 | 81 | −0.7 | #70.7 | 0 | 78.2 | 0.1 |
| 12938-13469 | 0.24 | −0.056 | 7.7 | 0.0027 | 78 | 1 | 80 | −1.7 | #70.7 | 0 | 77.2 | −0.9 |
| 12944-13108 | 0.24 | −0.056 | 7.4 | 0.02 | 78 | 1 | 81.5 | −0.2 | #70.7 | 0 | ND | ND |
| 12944-13471 | 0.24 | −0.056 | 8 | −0.012 | 78 | 1 | 81.3 | −0.4 | #70.7 | 0 | 77.9 | −0.2 |
| 12958-13079 | 0.26 | −0.088 | 7.2 | 0.035 | 75.2 | −1.8 | 81.2 | −0.5 | 69.6 | −1.1 | ND | ND |
| 12961-13093 | 0.26 | −0.088 | 7 | 0.046 | 75.2 | −1.8 | 81.3 | −0.4 | 69.6 | −1.1 | 77.9 | −0.2 |
| 13275-13461* | 0.094 | 0.35 | 6.9 | 0.051 | 75 | −2 | 81 | −0.7 | 70.4 | −0.3 | 78.2 | 0.1 |
| 13275-13470* | 0.094 | 0.35 | 7.7 | 0.0027 | 75 | −2 | 80 | −1.7 | 70.4 | −0.3 | 77.2 | −0.9 |
| 13276-13462* | 0.094 | 0.35 | 7.4 | 0.02 | 75 | −2 | 81.5 | −0.2 | 70.4 | −0.3 | ND | ND |
| 13276-13472* | 0.094 | 0.35 | 8 | −0.012 | 75 | −2 | 81.3 | −0.4 | 70.4 | −0.3 | 77.9 | −0.2 |
| 13308-13340 | 0.24 | −0.056 | 8 | −0.013 | 78 | 1 | 79 | −2.7 | #70.7 | 0 | 75.9 | −2.2 |
| 13308-13341 | 0.24 | −0.056 | 7.8 | −6.40E−04 | 78 | 1 | 78.8 | −2.9 | #70.7 | 0 | 76.1 | −2 |
| 13309-13425 | 0.24 | −0.056 | ND | ND | 78 | 1 | ND | ND | #70.7 | 0 | ND | ND |
| 13310-13444 | 0.24 | −0.056 | 8.1 | −0.015 | 78 | 1 | 80.2 | −1.5 | #70.7 | 0 | ND | ND |
| 13314-13424 | 0.18 | 0.075 | 7.9 | −0.007 | 75.7 | −1.3 | 81.3 | −0.4 | 70.8 | 0.1 | 77.8 | −0.3 |
| 13314-13438 | 0.18 | 0.075 | 7.2 | 0.035 | 75.7 | −1.3 | 81.2 | −0.5 | 70.8 | 0.1 | ND | ND |
| 13316-13451 | 0.18 | 0.075 | 7 | 0.046 | 75.7 | −1.3 | 81.3 | −0.4 | 70.8 | 0.1 | 77.9 | −0.2 |
| 13322-13443 | 0.26 | −0.088 | ND | ND | 75.2 | −1.8 | ND | ND | 69.6 | −1.1 | ND | ND |
| 13323-13444 | 0.26 | −0.088 | 8.1 | −0.015 | 75.2 | −1.8 | 80.2 | −1.5 | 69.6 | −1.1 | ND | ND |

H1L1 Fab is CAT-2200 and the H2L2 Fab is Pertuzumab.
A number of heterodimers had precipitate associated with them, noted with
*in Tables 9A and 9B if the precipitate was associated with the H1L1 Fab; noted with
“†” in Tables 9A and 9B is the precipitate was associated with H2L2 Fab: and noted with
“‡” in Tables 9A and 9B if the precipitate was associated svith both the H1L1 and H2L2 Fabs.
#denotes the presence of a shoulder to the main Fab peak (transition) in DSC thermogram

TABLE 10-A1
Cluster 1 K-L designs

| Unique identifier | H1 mutation (Kabat) | L1 mutation (Kabat) | H2 mutation (Kabat) | L2 mutation (Kabat) |
|---|---|---|---|---|
| 13173-13477 | A125R_L143D_K145T | S122D_E124Q_T131K_V133S | L143I_S186K_K228D | S121K_Q124E_V133D |
| 13173-13506 | A125R_L143D_K145T | S122D_E124Q_T131K_V133S | S186K_K228D | S121K_Q124E_V133D |
| 13180-13515 | A125R_L143E_K145T_Q179E | S122D_E124Q_T131R | S188K_K228D | S121K_V133I_S176D_T178E |
| 13177-13513 | A125R_L143D_K145T | S122D_E124Q_T131R | S188K_K228D | S121K_S131D_V133G_T178F |
| 13178-13417 | A125R_L143D_K145T | S122D_E124Q_T131R | L124R_S186R_K228D | S121K_V133G_S176D_T180E |
| 13171-13416 | A125R_L143D_K145T | S122D_E124Q_T131K | L124R_S186R_K228D | S121K_V133G_S176D_T180E |
| 13176-13487 | A125R_L143D_K145T | S122D_E124Q_T131R | L143R_K228D | S121K_Q124E_V133D_T180D |
| 13175-13486 | A125R_L143D_K145T | S122D_E124Q_T131R | L143R_K228D | S121K_Q124E_V133D |

TABLE 10-A2
Cluster 2 K-L designs

| Unique identifier | H1 mutation (Kabat) | L1 mutation (Kabat) | H2 mutation (Kabat) | L2 mutation (Kabat) |
|---|---|---|---|---|
| 12906-13152 | L143D_K145T | E124Q_T131K_V133S | S186K | Q124E_V133D |
| 10970-11348 | L143D_K145T_Q179E | E124Q_T131K | S186K | V133D_Q160E |
| 10969-11344 | L143D_K145T_Q179E | E124Q_T131K | S186K | V133D |
| 10969-11290 | L143D_K145T_Q179E | E124Q_T131K | L143I_S186K | V133D |
| 10966-11351 | L143D_K145T | T131K_V133S | S186K | V133D_Q160E |
| 10966-11297 | L143D_K145T | T131K_V133S | L143I_S186K | V133D_Q160E |
| 10965-11347 | L143D_K145T | T131K_V133S | S186K | V133D |
| 10965-11293 | L143D_K145T | T131K_V133S | L143I_S186K | V133D |
| 10962-11350 | L143D_K145T | T131K | S186K | V133D_Q160E |
| 10962-11296 | L143D_K145T | T131K | L143I_S186K | V133D_Q160E |
| 10946-11382 | L143D_K145T | E124Q_T131R | S188K | S131E_V133G_T178F |
| 10945-11377 | L143D_K145T | E124Q_T131R | S188K | S131D_V133G_T178F |
| 10944-11314 | L143D_K145T | E124Q_T131R | L143R | Q124E_V133D |
| 10944-11307 | L143D_K145T | E124Q_T131R | L143K | Q124E_V133D |
| 10943-11349 | L143D_K145T | E124Q_T131K_V133S | S186K | V133D_Q160E |
| 10943-11295 | L143D_K145T | E124Q_T131K_V133S | L143I_S186K | V133D_Q160E |
| 10942-11345 | L143D_K145T | E124Q_T131K_V133S | S186K | V133D |
| 10942-11291 | L143D_K145T | E124Q_T131K_V133S | L143I_S186K | V133D |
| 10928-11348 | L143D_K145T | E124Q_T131K | S186K | V133D_Q160E |
| 10928-11294 | L143D_K145T | E124Q_T131K | L143I_S186K | V133D_Q160E |
| 10927-11344 | L143D_K145T | E124Q_T131K | S186K | V133D |
| 10927-11290 | L143D_K145T | E124Q_T131K | L143I_S186K | V133D |
| 10924-11376 | L143D_K145T | E124Q_T131K | S188K | S131D_V133G_T178F |
| 10970-11294 | L143D_K145T_Q179E | E124Q_T131K | L143I_S186K | V133D_Q160E |
| 10973-11291 | L143D_K145T_Q179E | E124Q_T131K_V133S | L143I_S186K | V133D |
| 13279-13501 | L143D_K145T | E124Q_T131R | Q39E_L143R | Q38R_Q124E_V133D |
| 10973-11345 | L143D_K145T_Q179E | E124Q_T131K_V133S | S186K | V133D |
| 13278-13372 | L143D_K145T | E124Q_T131R | L45P_L143R | P44F_Q124E_V133D |
| 13216-13476 | A139W_L143D_K145T | E124Q_T131K_V133S | L143I_S186K | Q124E_V133D_L135W |
| 13283-13478 | L143D_K145T_F174G | E124Q_T131K_V133S_S176F | L143I_S186K_V190F | Q124E_V133D_L135A |
| 13216-13505 | A139W_L143D_K145T | E124Q_T131K_V133S | S186K | Q124E_V133D_L135W |
| 13283-13507 | L143D_K145T_F174G | E124Q_T131K_V133S_S176F | S186K_V190F | Q124E_V133D_L135A |
| 13220-13511 | A139W_L143D_K145T | E124Q_T131R | S188K | S131D_V133G_L135W_T178F |
| 13285-13414 | L143D_K145T_F174G | T116F_E124Q_T131K_S176F | L124R_S186K_V190F | V133G_L135A_S176D_T180E |
| 13287-13494 | L143D_K145T_F174G | T116F_E124Q_T131R_S176F | L143R_V190F | Q124E_V133D_L135A |
| 13219-13483 | A139W_L143D_K145T | E124Q_T131R | L143R | Q124E_V133D_L135W_T180D |
| 13218-13482 | A139W_L143D_K145T | E124Q_T131R | L143R | Q124E_V133D_L135W |
| 13282-13484 | L143D_K145T | E124Q_T131R | L143R | Q124E_V133D_T180D |
| 10982-11351 | L143D_K145T_Q179E | T131K_V133S | S186K | V133D_Q160E |
| 10982-11297 | L143D_K145T_Q179E | T131K_V133S | L143I_S186K | V133D_Q160E |
| 10981-11347 | L143D_K145T_Q179E | T131K_V133S | S186K | V133D |
| 10981-11293 | L143D_K145T_Q179E | T131K_V133S | L143I_S186K | V133D |
| 10978-11350 | L143D_K145T_Q179E | T131K | S186K | V133D_Q160E |
| 10978-11296 | L143D_K145T_Q179E | T131K | L143I_S186K | V133D_Q160E |
| 10977-11346 | L143D_K145T_Q179E | T131K | S186K | V133D |
| 10977-11292 | L143D_K145T_Q179E | T131K | L143I_S186K | V133D |
| 10974-11349 | L143D_K145T_Q179E | E124Q_T131K_V133S | S186K | V133D_Q160E |
| 10974-11295 | L143D_K145T_Q179E | E124Q_T131K_V133S | L143I_S186K | V133D_Q160E |
| 10923-11306 | L143D_K145T | E124Q_T131K | L143K | Q124E_V133D |

TABLE 10-A3
Cluster 3 K-L designs

| Unique identifier | H1 mutation (Kabat) | L1 mutation (Kabat) | H2 mutation (Kabat) | L2 mutation (Kabat) |
|---|---|---|---|---|
| 13290-13497 | L143E_K145T_Q179E | E124Q_T131R | Q39E_L124R_Q179K | Q38R_V133G_S176D_T180E |
| 10992-11410 | L143E_K145T_Q179E | E124Q_T131K | V177I_S188K | V133L_S176D_T178E |
| 10989-11259 | L143E_K145T_Q179E | E124Q_T131K | L124R_S186K | V133G_S176D_T180E |
| 10987-11261 | L143E_K145T_Q179E | E124Q_T131K | L124R_S186R | V133G_S176D_T178E |
| 10987-11257 | L143E_K145T_Q179E | E124Q_T131K | L124R_S186K | V133G_S176D_T178E |
| 10987-11249 | L143E_K145T_Q179E | E124Q_T131K | L124R_Q179K | V133G_S176D_T178E |
| 10986-11391 | L143E_K145T_Q179E | E124Q_T131K | S188K | S176E_T178E |
| 10985-11381 | L143E_K145T_Q179E | E124Q_T131K | S188K | S131E_V133G_T178F |
| 10984-11376 | L143E_K145T_Q179E | E124Q_T131K | S188K | S131D_V133G_T178F |
| 10983-11306 | L143E_K145T_Q179E | E124Q_T131K | L143K | Q124E_V133D |
| 10953-11411 | L143D_K145T | E124Q_T131R | V177I_S188K | V133L_S176D_T178E |
| 10952-11398 | L143D_K145T | E124Q_T131R | S188K | V133I_S176D_T178E |
| 10950-11264 | L143D_K145T | E124Q_T131R | L124R_S186R | V133G_S176D_T180E |
| 10950-11260 | L143D_K145T | E124Q_T131R | L124R_S186K | V133G_S176D_T180E |
| 10950-11252 | L143D_K145T | E124Q_T131R | L124R_Q179K | V133G_S176D_T180E |
| 10948-11262 | L143D_K145T | E124Q_T131R | L124R_S186R | V133G_S176D_T178E |
| 10948-11258 | L143D_K145T | E124Q_T131R | L124R_S186K | V133G_S176D_T178E |
| 10948-11250 | L143D_K145T | E124Q_T131R | L124R_Q179K | V133G_S176D_T178E |
| 10947-11392 | L143D_K145T | E124Q_T131R | S188K | S176E_T178E |
| 10934-11410 | L143D_K145T | E124Q_T131K | V177I_S188K | V133L_S176D_T178E |
| 10931-11263 | L143D_K145T | E124Q_T131K | L124R_S186R | V133G_S176D_T180E |
| 10931-11259 | L143D_K145T | E124Q_T131K | L124R_S186K | V133G_S176D_T180E |
| 10931-11251 | L143D_K145T | E124Q_T131K | L124R_Q179K | V133G_S176D_T180E |
| 10929-11261 | L143D_K145T | E124Q_T131K | L124R_S186R | V133G_S176D_T178E |
| 10929-11257 | L143D_K145T | E124Q_T131K | L124R_S186K | V133G_S176D_T178E |
| 10929-11249 | L143D_K145T | E124Q_T131K | L124R_Q179K | V133G_S176D_T178E |
| 10926-11391 | L143D_K145T | E124Q_T131K | S188K | S176E_T178E |
| 10989-11263 | L143E_K145T_Q179E | E124Q_T131K | L124R_S186R | V133G_S176D_T180E |
| 10998-11307 | L143E_K145T_Q179E | E124Q_T131R | L143K | Q124E_V133D |
| 13289-13368 | L143E_K145T_Q179E | E124Q_T131R | L45P_L124R_Q179K | P44F_V133G_S176D_T180E |
| 10998-11314 | L143E_K145T_Q179E | E124Q_T131R | L143R | Q124E_V133D |
| 13223-13512 | A139W_L143E_K145T_Q179E | E124Q_T131R | S188K | V133I_L135W_S176D_T178E |
| 13288-13516 | L143E_K145T_F174G_Q179E | T116F_E124Q_T131R_S176F | S188K_V190F | V133I_L135A_S176D_T178E |
| 13221-13411 | A139W_L143D_K145T | E124Q_T131R | L124R_S186K | V133G_L135W_S176D_T180E |
| 13214-13410 | A139W_L143D_K145T | E124Q_T131K | L124R_S186K | V133G_L135W_S176D_T180E |
| 13221-13406 | A139W_L143D_K145T | E124Q_T131R | L124R_Q179K | V133G_L135W_S176D_T180E |
| 13214-13405 | A139W_L143D_K145T | E124Q_T131K | L124R_Q179K | V133G_L135W_S176D_T180E |
| 13291-13488 | L143E_K145T_Q179E | E124Q_T131R | L143R_S188K | Q124E_V133D_S176D_T178D |
| 13281-13489 | L143D_K145T | E124Q_T131R | L143R_S188K | Q124E_V133D_S176D_T178E |
| 13280-13488 | L143D_K145T | E124Q_T131R | L143R_S188K | Q124E_V133D_S176D_T178D |
| 13295-13484 | L143E_K145T_Q179E | E124Q_T131R | L143R | Q124E_V133D_T180D |
| 13292-13489 | L143E_K145T_Q179E | E124Q_T131R | L143R_S188K | Q124E_V133D_S176D_T178E |
| 13294-13492 | L143E_K145T_Q179E | E124Q_T131R | L143R_S188K | Q124E_V133D_T178E |
| 13297-13402 | L143E_K145T_Q179E | E124Q_T131R | L124R_L143K | Q124E_V133G_S176D_T180E |
| 13296-13399 | L143E_K145T_Q179E | E124Q_T131R | L124R_L143K | Q124E_V133G_S176D |
| 11007-11411 | L143E_K145T_Q179E | E124Q_T131R | V177I_S188K | V133L_S176D_T178E |
| 11006-11398 | L143E_K145T_Q179E | E124Q_T131R | S188K | V133I_S176D_T178E |
| 11004-11264 | L143E_K145T_Q179E | E124Q_T131R | L124R_S186R | V133G_S176D_T180E |
| 11004-11260 | L143E_K145T_Q179E | E124Q_T131R | L124R_S186K | V133G_S176D_T180E |
| 11004-11252 | L143E_K145T_Q179E | E124Q_T131R | L124R_Q179K | V133G_S176D_T180E |
| 11002-11262 | L143E_K145T_Q179E | E124Q_T131R | L124R_S186R | V133G_S176D_T178E |
| 11002-11258 | L143E_K145T_Q179E | E124Q_T131R | L124R_S186K | V133G_S176D_T178E |
| 11002-11250 | L143E_K145T_Q179E | E124Q_T131R | L124R_Q179K | V133G_S176D_T178E |
| 11001-11392 | L143E_K145T_Q179E | E124Q_T131R | S188K | S176E_T178E |
| 11000-11382 | L143E_K145T_Q179E | E124Q_T131R | S188K | S131E_V133G_T178F |
| 10999-11377 | L143E_K145T_Q179E | E124Q_T131R | S188K | S131D_V133G_T178F |
| 10989-11251 | L143E_K145T_Q179E | E124Q_T131K | L124R_Q179K | V133G_S176D_T180E |

TABLE 10-A4
Cluster 4 K-L designs

| Unique identifier | H1 mutation (Kabat) | L1 mutation (Kabat) | H2 mutation (Kabat) | L2 mutation (Kabat) |
|---|---|---|---|---|
| 13330-13495 | S188K | K129T_S176E_Y178E | Q39E_L124E_K145T_Q179E | Q38R_S131R_V133G_S176R |
| 13329-13366 | S188K | K129T_S176E_Y178E | L45P_L124E_K145T_Q179E | P44F_S131R_V133G_S176R |
| 11108-11231 | S188K | S176E_Y178E | L124E_S188W | V133G_S176R_T178A |
| 11108-11227 | S188K | S176E_Y178E | L124E_S186I_S188W | V133G_S176R_T178A |
| 11107-11197 | S188K | S176E_Y178E | L124E | V133G_S176R |
| 11106-11229 | S188K | S176E_Y178E | L124E_S188W | V133A_S176K_T178A |
| 11106-11225 | S188K | S176E_Y178E | L124E_S186I_S188W | V133A_S176K_T178A |
| 11105-11195 | S188K | S176E_Y178E | L124E | V133A_S176K |
| 11104-11190 | S188K | S176E_Y178E | K145T_V177D_S188D | S176K_T178R |
| 11103-11186 | S188K | S176E_Y178E | K145T_V177D_S188D | S176K_T178L |
| 11102-11180 | S188K | S176E_Y178E | K145T_V177D_S188D | S176K_T178K |
| 11101-11219 | S188K | S176E_Y178E | L124E_K145T_Q179E_S188W | S131R_V133G_S176R_T178A |
| 11101-11213 | S188K | S176E_Y178E | L124E_K145T_Q179E_S186I_S188W | S131R_V133G_S176R_T178A |
| 11100-11205 | S188K | S176E_Y178E | L124E_K145T_Q179E | S131R_V133G_S176R |
| 11099-11216 | S188K | S176E_Y178E | L124E_K145T_Q179E_S188W | S131K_V133G_S176R_T178A |
| 11099-11210 | S188K | S176E_Y178E | L124E_K145T_Q179E_S186I_S188W | S131K_V133G_S176R_T178A |
| 11098-11200 | S188K | S176E_Y178E | L124E_K145T_Q179E | S131K_V133G_S176R |
| 11076-11355 | S188A | S176A_Y178W | S186L_S188W | S176A_T178A |
| 10771-11360 | — | — | S186L_S188W | S176V |
| 10771-11335 | — | — | S186I_S188W | S176V |
| 11163-11194 | V177I_S188K | V133L_S176D_Y178E | K145T_V177D_S188D | S176K_T178R |
| 11162-11184 | V177I_S188K | V133L_S176D_Y178E | K145T_V177D_S188D | S176K_T178K |
| 11144-11192 | V177I_S188K | V133I_S176D_Y178E | K145T_V177D_S188D | S176K_T178R |
| 11143-11187 | V177I_S188K | V133I_S176D_Y178E | K145T_V177D_S188D | S176K_T178L |
| 11142-11182 | V177I_S188K | V133I_S176D_Y178E | K145T_V177D_S188D | S176K_T178K |
| 11089-11189 | S188K | S176D_Y178E | K145T_V177D_S188D | S176K_T178R |
| 11088-11185 | S188K | S176D_Y178E | K145T_V177D_S188D | S176K_T178L |
| 11124-11202 | S188K | V133I_S176D_Y178E | L124E_K145T_Q179E | S131K_V133G_S176R |
| 11125-11207 | S188K | V133I_S176D_Y178E | L124E_K145T_Q179E | S131R_V133G_S176R |
| 11140-11202 | V177I_S188K | V133I_S176D_Y178E | L124E_K145T_Q179E | S131K_V133G_S176R |
| 11166-11198 | V177I_S188K | V133L_S176D_Y178E | L124E | V133G_S176R |
| 13333-13387 | S188K | K129T_S176E_Y178E | L124E_K145T_Q179E | S131R_V133G_S176R |
| 13207-13363 | A125R_S188K | S122D_K129T_S176D_Y178T | K145T_S186E_K228D | S121K_S131K_S176K |
| 13209-13364 | A125R_S188K | S122D_K129T_S176E_Y178E | K145T_S186E_K228D | S121K_S131K_S176K |
| 13235-13357 | A139W_S188K | K129T_S176D_Y178T | K145T_S186E | S131K_L135W_S176K |
| 13237-13358 | A139W_S188K | K129T_S176E_Y178E | K145T_S186E | S131K_L135W_S176K |
| 13337-13362 | S188K | K129T_Y178E | K145T_S186E | S131K_S176K |
| 13335-13361 | S188K | K129T_Y178D | K145T_S186E | S131K_S176K |
| 13328-13359 | S188K | K129T_S176D_Y178T | K145T_S186E | S131K_S176K |
| 13332-13360 | S188K | K129T_S176E_Y178E | K145T_S186E | S131K_S176K |
| 11167-11232 | V177I_S188K | V133L_S176D_Y178E | L124E_S188W | V133G_S176R_T178A |
| 11167-11228 | V177I_S188K | V133L_S176D_Y178E | L124E_S186I_S188W | V133G_S176R_T178A |
| 11165-11230 | V177I_S188K | V133L_S176D_Y178E | L124E_S188W | V133A_S176K_T178A |
| 11141-11207 | V177I_S188K | V133I_S176D_Y178E | L124E_K145T_Q179E | S131R_V133G_S176R |
| 11165-11226 | V177I_S188K | V133L_S176D_Y178E | L124E_S186I_S188W | V133A_S176K_T178A |
| 11164-11196 | V177I_S188K | V133L_S176D_Y178E | L124E | V133A_S176K |
| 11161-11220 | V177I_S188K | V133L_S176D_Y178E | L124E_K145T_Q179E_S188W | S131R_V133G_S176R_T178A |
| 11161-11214 | V177I_S188K | V133L_S176D_Y178E | L124E_K145T_Q179E_S188W | S131R_V133G_S176R_T178A |
| 11160-11208 | V177I_S188K | V133L_S176D_Y178E | L124E_K145T_Q179E | S131R_V133G_S176R |
| 11159-11217 | V177I_S188K | V133L_S176D_Y178E | L124E_K145T_Q179E_S188W | S131K_V133G_S176R_T178A |
| 11159-11211 | V177I_S188K | V133L_S176D_Y178E | L124E_K145T_Q179E_S186I_S188W | S131K_V133G_S176R_T178A |
| 11158-11203 | V177I_S188K | V133L_S176D_Y178E | L124E_K145T_Q179E | S131K_V133G_S176R |
| 11150-11193 | V177I_S188K | V133I_S176E_Y178E | K145T_V177D_S188D | S176K_T178R |
| 11149-11188 | V177I_S188K | V133I_S176E_Y178E | K145T_V177D_S188D | S176K_T178L |
| 11148-11183 | V177I_S188K | V133I_S176E_Y178E | K145T_V177D_S188D | S176K_T178K |
| 11087-11179 | S188K | S176D_Y178E | K145T_V177D_S188D | S176K_T178K |

TABLE 10-A5
Cluster 5 K-L designs

| Unique identifier | H1 mutation (Kabat) | L1 mutation (Kabat) | H2 mutation (Kabat) | L2 mutation (Kabat) |
|---|---|---|---|---|
| 11066-11181 | S186K_S188T | V133D_Y178T | K145T_V177D_S188D | S176K_T178K |
| 11063-11325 | S186K_S188T | V133D_Y178T | L143T_S188D | S131K_T178S |
| 11062-11323 | S186K_S188T | V133D_Y178T | L143T_S188D | S131K_S176A_T178S |
| 11056-11324 | S186K_S188T | T131S_V133D_Y178T | L143T_S188D | S131K_T178S |
| 11055-11322 | S186K_S188T | T131S_V133D_Y178T | L143T_S188D | S131K_S176A_T178S |
| 11044-11191 | L143S_S186K_S188T | V133D_Y178T | K145T_V177D_S188D | S176K_T178R |
| 11043-11181 | L143S_S186K_S188T | V133D_Y178T | K145T_V177D_S188D | S176K_T178K |
| 10898-11239 | L124E_V190E | V133I_L135R_Y178F | L124R | V133G_S176D |
| 10888-11415 | L124E_V190D | V133I_L135K_Y178F | V177I_S188K | V133L_S176D_T178E |
| 10883-11236 | L124E_V190D | V133I_L135K | L124R | V133G_S176D |
| 10881-11412 | L124E_V190D | L135K_Y178F | V177I_S188K | V133L_S176D_T178E |

TABLE 10-A6
Cluster 6 K-L designs

| Unique identifier | H1 mutation (Kabat) | L1 mutation (Kabat) | H2 mutation (Kabat) | L2 mutation (Kabat) |
|---|---|---|---|---|
| 10861-11409 | K145T_V177D_S188D | S176K_Y178R | V177I__S188K | V133I_S176E_T178E |
| 10776-11353 | D146T_V177D_S188D | S176K_Y178R | S186K_S188T | V133D |
| 10814-11233 | K145T_V177D_S188D | S176K_Y178K | L124R | V133G_S176D |
| 10813-11352 | K145T_V177D_S188D | S176K_Y178K | S186K_S188T | V133D |
| 10813-11320 | K145T_V177D_S188D | S176K_Y178K | L143S_S186K_S188T | V133D |
| 10813-11311 | K145T_V177D_S188D | S176K_Y178K | L143K | V133D |
| 10812-11393 | K145T_V177D_S188D | S176K_Y178K | S188K | S176E_T178E |
| 10810-11383 | K145T_V177D_S188D | S176K_Y178K | S188K | S131E_V133G_T178F |
| 10808-11315 | K145T_V177D_S188D | S176K_Y178K | L143R | Q124E_V133D |
| 10808-11308 | K145T_V177D_S188D | S176K_Y178K | L143K | Q124E_V133D |
| 10806-11369 | K145T_V177D_S188D | S176K_Y178K | S186R | Q124E_Q160E_T180E |
| 10806-11326 | K145T_V177D_S188D | S176K_Y178K | Q179K | Q124E_Q160E_T180E |
| 10776-11321 | D146T_V177D_S188D | S176K_Y178R | L143S_S186K_S188T | V133D |
| 10816-11265 | K145T_V177D_S188D | S176K_Y178K | L124R_S186R | V133G_S176D_T180E |
| 10776-11312 | D146T_V177D_S188D | S176K_Y178R | L143K | V133D |
| 10862-11414 | K145T_V177D_S188D | S176K_Y178R | V177I_S188K | V133L_S176D_T178E |
| 10860-11406 | K145T_V177D_S188D | S176K_Y178R | V177I_S188K | V133I_S176D_T178E |
| 10853-11388 | K145T_V177D_S188D | S176K_Y178R | S188K | S176D_T178E |
| 10851-11380 | K145T_V177D_S188D | S176K_Y178R | S188K | S131D_V133G_T178F |
| 10833-11387 | K145T_V177D_S188D | S176K_Y178L | S188K | S176D_T178E |
| 10831-11379 | K145T_V177D_S188D | S176K_Y178L | S188K | S131D_V133G_T178F |
| 10820-11413 | K145T_V177D_S188D | S176K_Y178K | V177I_S188K | V133L_S176D_T178E |
| 10818-11404 | K145T_V177D_S188D | S176K_Y178K | V177I_S188K | V133I_S176D_T178E |
| 10811-11386 | K145T_V177D_S188D | S176K_Y178K | S188K | S176D_T178E |
| 10816-11253 | K145T_V177D_S188D | S176K_Y178K | L124R_Q179K | V133G_S176D_T180E |
| 10819-11407 | K145T_V177D_S188D | S176K_Y178K | V177I_S188K | V133I_S176E_T178E |
| 10858-11267 | K145T_V177D_S188D | S176K_Y178R | L124R_S186R | V133G_S176D_T180E |
| 10848-11371 | K145T_V177D_S188D | S176K_Y178R | S186R | Q124E_Q160E_T180E |
| 10858-11255 | K145T_V177D_S188D | S176K_Y178R | L124R_Q179K | V133G_S176D_T180E |
| 10857-11243 | K145T_V177D_S188D | S176K_Y178R | L124R | V133G_S176D_T178D |
| 10856-11235 | K145T_V177D_S188D | S176K_Y178R | L124R | V133G_S176D |
| 10855-11353 | K145T_V177D_S188D | S176K_Y178R | S186K_S188T | V133D |
| 10855-11321 | K145T_V177D_S188D | S176K_Y178R | L143S_S186K_S188T | V133D |
| 10855-11312 | K145T_V177D_S188D | S176K_Y178R | L143K | V133D |
| 13330-13495 | K145T_V177D_S188D | S176K_Y178R | S188K | S176E_T178E |
| 10852-11385 | K145T_V177D_S188D | S176K_Y178R | S188K | S131E_V133G_T178F |
| 10850-11317 | K145T_V177D_S188D | S176K_Y178R | L143R | Q124E_V133D |
| 10850-11310 | K145T_V177D_S188D | S176K_Y178R | L143K | Q124E_V133D |
| 10848-11328 | K145T_V177D_S188D | S176K_Y178R | Q179K | Q124E_Q160E_T180E |
| 10827-11373 | K145T_V177D_S188D | S176K_Y178L | S186R_S188W | Q124E_Q160E_S176A_T178A_T180E |
| 10840-11408 | K145T_V177D_S188D | S176K_Y178L | V177I_S188K | V133I_S176E_T178E |
| 10837-11266 | K145T_V177D_S188D | S176K_Y178L | L124R_S186R | V133G_S176D_T180E |
| 10837-11254 | K145T_V177D_S188D | S176K_Y178L | L124R_Q179K | V133G_S176D_T180E |
| 10835-11234 | K145T_V177D_S188D | S176K_Y178L | L124R | V133G_S176D |
| 10834-11394 | K145T_V177D_S188D | S176K_Y178L | S188K | S176E_T178E |
| 10832-11384 | K145T_V177D_S188D | S176K_Y178L | S188K | S131E_V133G_T178F |
| 10830-11316 | K145T_V177D_S188D | S176K_Y178L | L143R | Q124E_V133D |
| 10830-11309 | K145T_V177D_S188D | S176K_Y178L | L143K | Q124E_V133D |
| 10828-11370 | K145T_V177D_S188D | S176K_Y178L | S186R | Q124E_Q160E_T180E |
| 10828-11327 | K145T_V177D_S188D | S176K_Y178L | Q179K | Q124E_Q160E_T180E |
| 10809-11378 | K145T_V177D_S188D | S176K_Y178K | S188K | S131D_V133G_T178F |

TABLE 10-A7
Cluster 7 K-L designs

| Unique identifier | H1 mutation (Kabat) | L1 mutation (Kabat) | H2 mutation (Kabat) | L2 mutation (Kabat) |
|---|---|---|---|---|
| 13253-13498 | K145T_S188E | Y178K | Q39E_L124R_Q179K | Q38R_V133G_S176D_T180E |
| 13252-13369 | K145T_S188E | Y178K | L45P_L124R_Q179K | P44F_V133G_S176D_T180E |
| 13258-13504 | K145T_S188E | Y178K | Q179K_S188K | Q124E_S176E_T180E |
| 13261-13480 | K145T_S188E | Y178K | L143K_S188K | Q124E_V133D_S176E_T178E_T180E |
| 13262-13481 | K145T_S188E | Y178K | L143K_S188K | Q124E_V133D_S176E_T180E |
| 13255-13509 | K145T_S188E | Y178K | S186R_S188K | Q124E_Q160E_S176D_T178D_T180E |
| 13254-13508 | K145T_S188E | Y178K | S186R_S188K | Q124E_Q160E_S176D_T178D |
| 10875-11416 | K145T_V177T_S188E | Y178K | V177I_S188K | V133L_S176D_T178E |
| 10873-11268 | K145T_V177T_S188E | Y178K | L124R_S186R | V133G_S176D_T180E |
| 10873-11256 | K145T_V177T_S188E | Y178K | L124R_Q179K | V133G_S176D_T180E |
| 10871-11240 | K145T_V177T_S188E | Y178K | L124R | V133G_S176D |
| 10870-11396 | K145T_V177T_S188E | Y178K | S188K | S176E_T178E |
| 10869-11390 | K145T_V177T_S188E | Y178K | S188K | S176D_T178E |
| 10800-11416 | K145T_S188E | Y178K | V177I_S188K | V133L_S176D_T178E |
| 10798-11268 | K145T_S188E | Y178K | L124R_S186R | V133G_S176D_T180E |
| 10798-11256 | K145T_S188E | Y178K | L124R_Q179K | V133G_S176D_T180E |
| 10796-11396 | K145T_S188E | Y178K | S188K | S176E_T178E |
| 13256-13502 | K145T_S188E | Y178K | Q179K_S188K | Q124E_S176E_T178D_T180E |
| 13257-13503 | K145T_S188E | Y178K | Q179K_S188K | Q124E_S176E_T178E_T180E |
| 13269-13403 | K145T_S188E | Y178K | L124R_L143K | Q124E_V133G_S176D_T180E |
| 13264-13400 | K145T_S188E | Y178K | L124R_L143K | Q124E_V133G_S176D |
| 13169-13418 | A125R_K145T_S188E | S122D_Y178K | L124R_S186R_K228D | S121K_V133G_S176D_T180E |
| 13212-13415 | A139W_K145T_S188E | Y178K | L124R_S186R | V133G_L135W_S176D_T180E |
| 13259-13491 | K145T_S188E | Y178K | L143R_S188K | Q124E_V133D_S176D_T178E |
| 13263-13493 | K145T_S188E | Y178K | L143R_S188K | Q124E_V133D_T178E |
| 13167-13514 | A125R_K145T_S188E | S122D_Y178K | S188K_K228D | S121K_S176E_T178E |
| 13210-13510 | A139W_K145T_S188E | Y178K | S188K | L135W_S176E_T178E |
| 13168-13397 | A125R_K145T_S188E | S122D_Y178K | L124R_K228D | S121K_V133G_S176D |
| 13268-13401 | K145T_S188E | Y178K | L124R_L143K | Q124E_V133G_S176D_T178E_T180E |
| 13211-13395 | A139W_K145T_S188E | Y178K | L124R | V133G_L135W_S176D |
| 13212-13396 | A139W_K145T_S188E | Y178K | L124R | V133G_L135W_S176D_T180E |
| 13265-13419 | K145T_S188E | Y178K | L124R_S188K | Q124E_V133G_S176D_T178D |
| 13265-13392 | K145T_S188E | Y178K | L124K_S188K | Q124E_V133G_S176D_T178D |
| 10798-13036 | K145T_S188E | Y178K | L124R | V133G_S176D_T180E |
| 12878-11240 | K145T_S188E | Y178K | L124R | V133G_S176D |
| 10795-11390 | K145T_S188E | Y178K | S188K | S176D_T178E |

TABLE 10-A8
Cluster 8 K-L designs

| Unique identifier | H1 mutation (Kabat) | L1 mutation (Kabat) | H2 mutation (Kabat) | L2 mutation (Kabat) |
|---|---|---|---|---|
| 13318-13437 | S186K | K129T_V133D_Y178T | L143E_K145T_Q179E | Q1248_Q160K_T178R |
| 11061-11273 | S186K_S188T | V133D_Y178T | L143E_K145T_Q179E | Q1248_Q160K_T178R |
| 12938-13064 | Q179K | K129T_S180E | L143E_K145T | Q1248_Q160K_T178R |
| 13319-13473 | S186R | K129T_S176A_Y178W_S180E | L143E_K145T_S188W | Q124K_S176A_T178R |
| 13277-13420 | L143A_S186R | K129T_V133W_S180E | L124W_L143E_K145T | Q124K_V133A_T178R |
| 13322-13425 | S186R | K129T_S180E | L143E_K145T | Q124R_T129K_Q160K_T178R |
| 12961-13471 | S186R | K129T_S180E | L143E_K145T_S188L | Q124R_T178R |
| 12961-13108 | S186R | K129T_S180E | L143E_K145T_Q179E_S188L | Q124R_T178R |
| 12958-13469 | S186R | K129T_S180E | L143E_K145T_S188L | Q124R_Q160K_T178R |
| 12958-13106 | S186R | K129T_S180E | L143E_K145T_Q179E_S188L | Q124R_Q160K_T178R |
| 12958-13064 | S186R | K129T_S180E | L143E_K145T | Q124R_Q160K_T178R |
| 12944-13093 | Q179K | K129T_S180E | L143E_K145T_Q179E | Q124R_T178R |
| 11157-11279 | V177I_S188K | V133L_S176D_Y178E | L143E_K145T_Q179E | Q124R_T178R |
| 11156-11274 | V177I_S188K | V133L_S176D_Y178E | L143E_K145T_Q179E | Q124R_Q160K_T178R |
| 11097-11277 | S188K | S176E_Y178E | L143E_K145T_Q179E | Q124R_T178R |
| 11096-11272 | S188K | S176E_Y178E | L143E_K145T_Q179E | Q124R_Q160K_T178R |
| 11042-11273 | L143S_S186K_S188T | V133D_Y178T | L143E_K145T_Q179E | Q124R_Q160K_T178R |
| 13314-13424 | Q179K | K129T_Y178E_S180E | L143E_K145T | Q124R_Q160K_T178R |
| 11030-11285 | L143K | K129T_V133D | L143E_K145T_Q179E_S188L | Q124R_T178R |
| 11030-11276 | L143K | K129T_V133D | L143E_K145T_Q179E | Q124R_T178R |
| 11029-11288 | L143K | K129T_V133D | L143E_K145T_Q179E_S188W | Q124R_S176A_T178R |
| 11029-11282 | L143K | K129T_V133D | L143E_K145T_Q179E_S186I_S188W | Q124R_S176A_T178R |
| 11028-11275 | L143K | K129T_V133D | L143E_K145T_Q179E | Q124R_Q160R_T178R |
| 11027-11287 | L143K | K129T_V133D | L143E_K145T_Q179E_S188W | Q124R_Q160R_S176A_T178R |
| 11027-11281 | L143K | K129T_V133D | L143E_K145T_Q179E_S186I_S188W | Q124R_Q160R_S176A_T178R |
| 11026-11270 | L143K | K129T_V133D | L124W_L143E_K145T_Q179E | Q124K_V133A_T178R |
| 11025-11284 | L143K | K129T_V133D | L143E_K145T_Q179E_S188L | Q124K_T178R |
| 13308-13341 | Q179K | K129T_S180E | F122C_L143E_K145T_Q179E | Q124C_Q160K_T178R |
| 13275-13461 | L143A_Q179K | K129T_V133W_S180E | L143E_K145T_Q179E_S188L | Q124R_Q160K_T178R |
| 12938-13106 | Q179K | K129T_S180E | L143E_K145T_Q179E_S188L | Q124R_Q160K_T178R |
| 13314-13438 | Q179K | K129T_Y178E_S180E | L143E_K145T_Q179E | Q124R_Q160K_T178R |
| 12938-13079 | Q179K | K129T_S180E | L143E_K145T_Q179E | Q124R_Q160K_T178R |
| 13309-13425 | Q179K | K129T_S180E | L143E_K145T | Q124R_T129K_Q160K_T178R |
| 12938-13469 | Q179K | K129T_S180E | L143E_K145T_S188L | Q124R_Q160K_T178R |
| 12957-13061 | S186R | K129T_S180E | L143E_K145T | Q124K_T178R |
| 13336-13450 | S188K | K129T_Y178E | L143E_K145T_Q179E | Q124R_T178R |
| 13321-13499 | S186R | K129T_S180E | Q39E_L143E_K145T_Q179E | Q38R_Q124K_T178R |
| 13339-13500 | S188K | S176E_Y178E | Q39E_L143E_K145T_Q179E | Q38R_Q124R_T178R |
| 13320-13370 | S186R | K129T_S180E | L45P_L143E_K145T_Q179E | P44F_Q124K_T178R |
| 13338-13371 | S188K | S176E_Y178E | L45P_L143E_K145T_Q179E | P44F_Q124R_T178R |
| 13224-13428 | A139W_L143I_S186K | K129T_V133D_Y178T | L143E_K145T_Q179E | Q124R_L135W_Q160K_T178R |
| 13229-13428 | A139W_S186K | K129T_V133D_Y178T | L143E_K145T_Q179E | Q124R_L135W_Q160K_T178R |
| 13226-13434 | A139W_L143K | K129T_V133D | L143E_K145T_Q179E | Q124R_L135W_T178R |
| 13305-13448 | L143K_V190K | K129T_V133D_L1355 | L143E_K145T_Q179E | Q124R_T178R |
| 13273-13441 | L124K_L143K | K129T_T131E_V133D | L143E_K145T_Q179E | Q124R_Q160R_T178R |
| 13271-13439 | L124K_L143K | K129T_T131D_V133D | L143E_K145T_Q179E | Q124R_Q160R_T178R |
| 13300-13441 | L143K | K129T_T131E_V133D | L143E_K145T_Q179E | Q124R_Q160R_T178R |
| 13298-13439 | L143K | K129T_T131D_V133D | L143E_K145T_Q179E | Q124R_Q160R_T178R |
| 13234-13432 | A139W_S188K | K129T_S176D_Y178T | L143E_K145T_Q179E | Q124R_L135W_T178R |
| 13236-13433 | A139W_S188K | K129T_S176E_Y178E | L143E_K145T_Q179E | Q124R_L135W_T178R |
| 13334-13449 | S188K | K129T_Y178D | L143E_K145T_Q179E | Q124R_T178R |
| 12944-13471 | Q179K | K129T_S180E | L143E_K145T_S188L | Q124R_T178R |
| 13327-13446 | S188K | K129T_S176D_Y178T | L143E_K145T_Q179E | Q124R_T178R |
| 13331-13447 | S188K | K129T_S176E_Y178E | L143E_K145T_Q179E | Q124R_T178R |
| 13276-13462 | L143A_Q179K | K129T_V133W_S180E | L143E_K145T_Q179E_S188L | Q124R_T178R |
| 12944-13108 | Q179K | K129T_S180E | L143E_K145T_Q179E_S188L | Q124R_T178R |
| 13316-13451 | Q179K | K129T_Y178E_S180E | L143E_K145T_Q179E | Q124R_T178R |
| 13322-13443 | S186R | K129T_S180E | L143E_K145T_Q179E | Q124R_T129K_Q160K_T178R |
| 13310-13444 | Q179K | K129T_S180E | L143E_K145T_Q179E | Q124R_T129K_T178R |
| 13323-13444 | S186R | K129T_S180E | L143E_K145T_Q179E | Q124R_T129K_T178R |
| 12958-13079 | S186R | K129T_S180E | L143E_K145T_Q179E | Q124R_Q160K_T178R |
| 12961-13093 | S186R | K129T_S180E | L143E_K145T_Q179E | Q124R_T178R |
| 13317-13423 | Q179K_V190K | K129T_V133D_L135S_S180E | L143E_K145T | Q124R_Q160K_T178R |
| 13308-13340 | Q179K | K129T_S180E | F122C_L143E_K145T | Q124C_Q160K_T178R |
| 13276-13472 | L143A_Q179K | K129T_V133W_S180E | L143E_K145T_S188L | Q124R_T178R |
| 13275-13470 | L143A_Q179K | K129T_V133W_S180E | L143E_K145T_S188L | Q124R_Q160K_T178R |
| 13309-13443 | Q179K | K129T_S180E | L143E_K145T_Q179E | Q124R_T129K_Q160K_T178R |

TABLE 10-A9
Cluster 9 K-L designs

| Unique identifier | H1 mutation (Kabat) | L1 mutation (Kabat) | H2 mutation (Kabat) | L2 mutation (Kabat) |
|---|---|---|---|---|
| 13182-13458 | A125R_L143K | S122D_K129T_V133D | L143E_K145T_Q179E_K228D | S121K_Q124R_T178R |
| 13206-13454 | A125R_S188K | S122D_K129T_S176D_Y178T | L143E_K145T_Q179E_K228D | S121K_Q124R_T178R |
| 13197-13353 | A125R_Q179K | S122D_K129T_S180E | K145T_Q179E_K228D | S121K_T129K_S131K |
| 13188-13430 | A125R_Q179K | S122D_K129T_S180E | L143E_K145T_Q179E | Q124R_L135W_Q160K_T178R |
| 13192-13452 | A125R_Q179K | S122D_K129T_S180E | L143E_K145T_Q179E_K228D | S121K_Q124R_Q160K_T178R |
| 13200-13421 | A125R_S186R | S122D_K129T_S180E | L143E_K145T_ | Q124R_L135W_Q160K_T178R |
| 13202-13426 | A125R_S186R | S122D_K129T_S180E | L143E_K145T_K228D | S121K_Q124R_Q160K_T178R |
| 13203-13427 | A125R_S186R | S122D_K129T_S180E | L143E_K145T_K228D | S121K_Q124R_T178R |
| 13204-13351 | A125R_S186R | S122D_K129T_S180E | K145T_Q179E_K228D | S121K_S131K |
| 13205-13353 | A125R_S186R | S122D_K129T_S180E | K145T_Q179E_K228D | S121K_T129K_S131K |
| 13188-13421 | A125R_Q179K | S122D_K129T_S180E | L143E_K145T | Q124R_L135W_Q160K_T178R |
| 13190-13422 | A125R_Q179K | S122D_K129T_S180E | L143E_K145T | Q124R_L135W_T178R |
| 13192-13426 | A125R_Q179K | S122D_K129T_S180E | L143E_K145T_K228D | S121K_Q124R_Q160K_T178R |
| 13194-13427 | A125R_Q179K | S122D_K129T_S180E | L143E_K145T_K228D | S121K_Q124R_T178R |
| 13200-13430 | A125R_S186R | S122D_K129T_S180E | L143E_K145T_Q179E | Q124R_L135W_Q160K_T178R |
| 13194-13456 | A125R_Q179K | S122D_K129T_S180E | L143E_K145T_Q179E_K228D | S121K_Q124R_T178R |
| 13202-13452 | A125R_S186R | S122D_K129T_S180E | L143E_K145T_Q179E_K228D | S121K_Q124R_Q160K_T178R |
| 13203-13456 | A125R_S186R | S122D_K129T_S180E | L143E_K145T_Q179E_K228D | S121K_Q124R_T178R |
| 13208-13455 | A125R_S188K | S122D_K129T_S176E_Y178E | L143E_K145T_Q179E_K228D | S121K_Q124R_T178R |
| 13196-13351 | A125R_Q179K | S122D_K129T_S180E | K145T_Q179E_K228D | S121K_S131K |

TABLE 10-A10
Cluster 10 K-L designs

| Unique identifier | H1 mutation (Kabat) | L1 mutation (Kabat) | H2 mutation (Kabat) | L2 mutation (Kabat) |
|---|---|---|---|---|
| 13304-13466 | L143K_F174G | T116F_K129T_V133D_S176F | L143E_K145T_Q179E_V190F | Q124R_L135A_T178R |
| 13238-13459 | F174G_Q179K | T116F_K129T_S176F_S180F | L143E_K145T_Q179E_S188F_V190F | Q124K_L135A_T178R |
| 10780-11417 | F174G | S176F | V190F | |
| 10781-11300 | F174G | S176F | L143I_V190F | L135A |
| 10781-11419 | F174G | S176F | V190F | L135A |
| 10786-11299 | F174G | T116F_S176F | L143I_V190F | — |
| 10786-11418 | F174G | T116F_S176F | V190F | — |
| 10787-11301 | F174G | T116F_S176F | L143I_V190F | L135A |
| 10787-11420 | F174G | T116F_S176F | V190F | L135A |
| 10788-11422 | F174G | T116F_S176F | V190F | L135A_T178F |
| 10792-11305 | F174G | T116F_S176F | L143I_V190F | T178F |
| 10792-11424 | F174G | T116F_S176F | V190F | T178F |
| 10793-11375 | F174G | T116F_S176F | S188F | V133A |
| 10684-10706 | S186R | K129T_S180E | K145T_Q179E | S131K |
| 13248-13475 | F174G_S186R | T116F_K129T_S176F_S180E | L143E_K145T_V190F | Q124R_L135A_Q160K_T178R |
| 13250-13355 | F174G_S186R | T116F_K129T_S176F_S180E | K145T_Q179E_S188F_V190F | S131K_L135A |
| 13242-13475 | F174G_Q179K | T116F_K129T_S176F_S180E | L143E_K145T_V190F | Q124R_L135A_Q160K_T178R |
| 13246-13463 | F174G_S186R | T116F_K129T_S176F_S180E | L143E_K145T_Q179E_V190F | Q124K_L135A_T178R |
| 13238-13463 | F174G_Q179K | T116F_K129T_S176F_S180E | L143E_K145T_Q179E_V190F | Q124K_L135A_T178R |
| 13248-13465 | F174G_S186R | T116F_K129T_S176F_S180E | L143E_K145T_Q179E_V190F | Q124R_L135A_Q160K_T178R |
| 13246-13459 | F174G_S186R | T116F_K129T_S176F_S180E | L143E_K145T_Q179E_S188F_V190F | Q124K_L135A_T178R |
| 13242-13465 | F174G_Q179K | T116F_K129T_S176F_S180E | L143E_K145T_Q179E_V190F | Q124R_L135A_Q160K_T178R |

TABLE 10-A11
Cluster 11 K-L designs

| Unique identifier | H1 mutation (Kabat) | L1 mutation (Kabat) | H2 mutation (Kabat) | L2 mutation (Kabat) |
|---|---|---|---|---|
| 13185-13374 | A125R_L143K_V190K | S122D_K129T_V133D_L135S | L124E_A139I_K145T_V190I_K228D | S121K_S131K_L135K |
| 13306-13373 | L143K_V190K | K129T_V133D_L135S | L124E_A139I_K145T_V190I | S131K_L135K |
| 13306-13375 | L143K_V190K | K129T_V133D_L135S | L124E_K145T | S131K_L135K |

TABLE 10-A12
Cluster 12 K-L designs

| Unique identifier | H1 mutation (Kabat) | L1 mutation (Kabat) | H2 mutation (Kabat) | L2 mutation (Kabat) |
|---|---|---|---|---|
| 13303-13496 | L143K | K129T_V133D | Q39E_L124E_K145T_Q179E | Q38R_S131R_V133G_S176R |
| 13302-13367 | L143K | K129T_V133D | L45P_L124E_K145T_Q179E | P44F_S131R_V133G_S176R |
| 11033-11209 | L143K | K129T_V133D | L124E_K145T_Q179E_S186I_S188W | S131K_V133G_S176R_T178A |
| 11033-11215 | L143K | K129T_V133D | L124E_K145T_Q179E_S188W | S131K_V133G_S176R_T178A |
| 11034-11204 | L143K | K129T_V133D | L124E_K145T_Q179E | S131R_V133G_S176R |
| 11035-11212 | L143K | K129T_V133D | L124E_K145T_Q179E_S186I_S188W | S131R_V133G_S176R_T178A |
| 11035-11218 | L143K | K129T_V133D | L124E_K145T_Q179E_S188W | S131R_V133G_S176R_T178A |
| 11036-11221 | L143K | K129T_V133D | L124E_K145T_S186E | S131R_V133S_L135K |
| 11039-11224 | L143K | K129T_V133D | L124E_L143E_K145T | S131R_V133T_L135K_T178S |
| 11064-11201 | S186K_S188T | V133D_Y178T | L124E_K145T_Q179E | S131K_V133G_S176R |
| 11065-11206 | S186K_S188T | V133D_Y178T | L124E_K145T_Q179E | S131R_V133G_S176R |
| 13299-13379 | L143K | K129T_T131D_V133D | L124E_K145T_Q179E | S131K_V133G_S176R |
| 13301-13381 | L143K | K129T_T131E_V133D | L124E_K145T_Q179E | S131K_V133G_S176R |
| 13272-13379 | L124K_L143K | K129T_T131D_V133D | L124E_K145T_Q179E | S131K_V133G_S176R |
| 13274-13381 | L124K_L143K | K129T_T131E_V133D | L124E_K145T_Q179E | S131K_V133G_S176R |
| 13227-13383 | A139W_L143K | K129T_V133D | L124E_K145T_Q179E | S131R_V133G_L135W_S176R |
| 13184-13388 | A125R_L143K | S122D_K129T_V133D | L124E_K145T_Q179E_K228D | S121K_S131R_V133G_S176R |
| 13231-13386 | A139W_S186K_S188T | V133D_Y178T | L124E_K145T_Q179E | S131R_V133G_L135W_S176R |
| 13199-13391 | A125R_S186K_S188T | S122D_V133D_Y178T | L124E_K145T_Q179E_K228D | S121K_S131R_V133G_S176R |
| 13230-13384 | A139W_S186K | K129T_V133D_Y178T | L124E_K145T_Q179E | S131R_V133G_L135W_S176R |
| 13198-13389 | A125R_S186K | S122D_K129T_V133D_Y178T | L124E_K145T_Q179E_K228D | S121K_S131R_V133G_S176R |
| 13225-13384 | A139W_L143I_S186K | K129T_V133D_Y178T | L124E_K145T_Q179E | S131R_V133G_L135W_S176R |
| 13181-13389 | A125R_L143I_S186K | S122D_K129T_V133D_Y178T | L124E_K145T_Q179E_K228D | S121K_S131R_V133G_S176R |
| 11032-11199 | L143K | K129T_V133D | L124E_K145T_Q179E | S131K_V133G_S176R |

In Tables 10-A1 to 10-A12, H1L1 Fab is CAT-2200 and H2L2 Fab is Pertuzumab

TABLE 10-B1
Cluster 1 K-K-derived K-L designs

| Unique identifier | H1 mutation (Kabat) | L1 mutation (Kabat) | H2 mutation (Kabat) | L2 mutation (Kabat) |
|---|---|---|---|---|
| 10657-10760 | L143E_K145T_Q179E | E124K_Y178R | S186R | T178E_T180E |
| 10656-10758 | L143E_K145T_Q179E | E124K_Y178R | S186R | Q160E_T180E |

TABLE 10-B2
Cluster 2 K-K-derived K-L designs

| Unique identifier | H1 mutation (Kabat) | L1 mutation (Kubat) | H2 mutation (Kubat) | L2 mutation (Kubat) |
|---|---|---|---|---|
| 10652-10755 | L143E_K145T | E124R | S186R | Q124E_Q160E_T180E |
| 10652-10734 | L143E_K145T | E124R | Q179K | Q124E_Q160E_T180E |
| 10652-10702 | L143E_K145T | E124R | D146G_Q179K | Q124E_Q160E_T180E |

TABLE 10-B3
Cluster 3 K-K-derived K-L designs

| Unique identifier | H1 mutation (Kabat) | L1 mutation (Kabat) | H2 mutation (Kabat) | L2 mutation (Kabat) |
|---|---|---|---|---|
| 10690-10727 | S186R | Y178E_S180E | L143E_K145T_Q179E | Q124K_T178R |
| 10689-10707 | S186R | S180E | K145T_Q179E | S131K |
| 10685-10726 | S186R | S180E | L143E_K145T_Q179E | Q124K_T178R |
| 10685-10719 | S186R | S180E | L143E_K145T | Q124K_T178R |

TABLE 10-B4
Cluster 4 K-K-derived K-L designs

| Unique identifier | H1 mutation (Kabat) | L1 mutation (Kabat) | H2 mutation (Kabat) | L2 mutation (Kabat) |
|---|---|---|---|---|
| 10665-10724 | Q179K | S180E | L143E_K145T | Q124R_Q160K_T178R |
| 10664-10722 | Q179K | S180E | L143E_K145T | Q124R |
| 10632-10724 | D146G_Q179K | S180E | L143E_K145T | Q124R_Q160K_T178R |
| 10631-10722 | D146G_Q179K | S180E | L143E_K145T | Q124R |

TABLE 10-B5
Cluster 5 K-K-derived K-L designs

| | H1 | L1 | | |
|---|---|---|---|---|
| Unique | mutation | mutation | H2 mutation | L2 mutation |
| identifier | (Kabat) | (Kabat) | (Kabat) | (Kabat) |
| 10688-10724 | S186R | S180E | L143E_K145T | Q124R_Q160K_T178R |
| 10687-10722 | S186R | S180E | L143E_K145T | Q124R |
| 10683-10720 | S186R | | L143E_K145T | Q124R |
| 10682-10718 | S186R | | L143E_K145T | Q124K_T178R |
| 10659-10720 | L143R | | L143E_K145T | Q124R |
| 10658-10720 | L143K | | L143E_K145T | Q124R |

TABLE 10-B6
Cluster 6 K-K-derived K-L designs

| Unique | H1 mutation | L1 mutation | H2 mutation | L2 mutation |
|---|---|---|---|---|
| identifier | (Kabat) | (Kabat) | (Kabat) | (Kabat) |
| 10681-10747 | Q39R | Q38E | Q39E | Q38R |
| 10681-10741 | Q39R | Q38E | Q39D | Q38R |
| 10680-10746 | Q39R | Q38D | Q39E | Q38R |
| 10680-10740 | Q39R | Q38D | Q39D | Q38R |
| 10679-10738 | Q39K | Q38E | Q39D | Q38K |
| 10678-10743 | Q39K | Q38D | Q39E | Q38K |
| 10677-10751 | Q39E | Q38R | Q39R | Q38E |
| 10676-10750 | Q39E | Q38R | Q39R | Q38D |
| 10671-10751 | Q39D | Q38R | Q39R | Q38E |
| 10623-10745 | — | — | Q39E | Q38R |
| 10623-10739 | — | — | Q39D | Q38R |
| 10622-10742 | — | — | Q39E | Q38K |
| 10622-10736 | — | — | Q39D | Q38K |
| 10679-10744 | Q39K | Q38E | Q39E | Q38K |
| 10678-10737 | Q39K | Q38D | Q39D | Q38K |
| 10674-10749 | Q39E | Q38K | Q39K | Q38E |

TABLE 10-B7
Cluster 7 K-K-derived K-L designs

| Unique | H1 mutation | L1 mutation | H2 mutation | L2 mutation |
|---|---|---|---|---|
| identifier | (Kabat) | (Kabat) | (Kabat) | (Kabat) |
| 10625-10701 | — | L135W | A139W | F116A_L135V |
| 10624-10700 | — | L135W | A139W | F116A |

TABLE 10-B8
Cluster 8 K-K-derived K-L designs

| Unique | H1 mutation | L1 mutation | H2 mutation | L2 mutation |
|---|---|---|---|---|
| identifier | (Kabat) | (Kabat) | (Kabat) | (Kabat) |
| 10662-10733 | L45F | — | L45P | P44F |
| 10662-10731 | L45F | — | L45A | P44F |
| 10621-10733 | — | — | L45P | P44F |
| 10621-10731 | — | — | L45A | P44F |

TABLE 10-B9
Cluster 9 K-K-derived K-L designs

| Unique | H1 mutation | L1 mutation | H2 mutation | L2 mutation |
|---|---|---|---|---|
| identifier | (Kabat) | (Kabat) | (Kabat) | (Kabat) |
| 10629-10695 | A139W | T116A | | L135W |

TABLE 10-B10
Cluster 10 K-K-derived K-L designs

| Unique | H1 mutation | L1 mutation | H2 mutation | L2 mutation |
|---|---|---|---|---|
| identifier | (Kabat) | (Kabat) | (Kabat) | (Kabat) |
| 10640-10713 | L124E | V133G_S176R | L124R | V133G_S176D |

TABLE 11A
Amino acid mutations in the Fab regions of H1, L1, H2 and L2
for K-L designs tested in SMCA format (Kabat numbering).

| Cluster | Design | H1 mutation (lambda) | L1 mutation (lambda) | H2 mutation (kappa) | L2 mutation (kappa) |
|---|---|---|---|---|---|
| kl-6 | 2892 | K145T_V177D_S188D | S176K_Y178K | L143K | Q124E_V133D |
| kl-6 | 2901 | K145T_V177D_S188D | S176K_Y178K | L124R | V133G_S176D |
| kl-4 | 2979 | S188K | S176E_Y178E | L124E_K145T_Q179E | S131R_V133G_S176R |
| kl-3 | 2994 | L143E_K145T_Q179E | E124Q_T131K | L143K | Q124E_V133D |
| kl-3 | 3008 | L143E_K145T_Q179E | E124Q_T131K | L124R_S186K | V133G_S176D_T178E |
| kl-3 | 3018 | L143D_K145T | E124Q_T131K | L124R_S186R | V133G_S176D_T180E |
| kl-2 | 3025 | L143D_K145T | E124Q_T131R | S188K | S131D_V133G_T178F |
| kl-3 | 3041 | L143D_K145T | E124Q_T131R | S188K | S176E_T178E |
| kl-12 | 3102 | L143K | K129T_V133D | L124E_K145T_Q179E | S131R_V133G_S176R |
| kl-8 | 3109 | L143K | K129T_V133D | L124W_L143E_K145T_Q179E | Q124K_V133A_T178R |
| kl-12 | 3113 | S186K_S188T | V133D_Y178T | L124E_K145T_Q179E | S131R_V133G_S176R |
| kl-5 | 3122 | S186K_S188T | V133D_Y178T | K145T_V177D_S188D | S176K_T178K |
| kl-8 | 3878 | Q179K | K129T_S180E | L143E_K145T_S188L | Q124R_Q160K_T178R |
| kl-9 | 3890 | A125R_Q179K | S122D_K129T_S180E | L143E_K145T_K228D | S121K_Q124R_T178R |
| kl-8 | 3898 | Q179K | K129T_Y178E_S180E | L143E_K145T_Q179E | Q124R_T178R |
| kl-10 | 3910 | F174G_Q179K | T116F_K129T_S176F_S180E | L143E_K145T_Q179E_V190F | Q124K_L135A_T178R |
| kl-7 | 3931 | K145T_S188E | Y178K | L124R | V133G_S176D |
| kl-4 | 3947 | S188K | K129T_Y178D | K145T_S186E | S131K_S176K |
| kl-4 | 3951 | A125R_S188K | S122D_K129T_S176E_Y178E | K145T_S186E_K228D | S121K_S131K_S176K |
| kl-7 | 3954 | A125R_K145T_S188E | S122D_Y178K | S188K_K228D | S121K_S176E_T178E |
| kl-7 | 3957 | K145T_S188E | Y178K | L143R_S188K | Q124E_V133D_T178E |
| kl-9 | 3967 | A125R_S188K | S122D_K129T_S176E_Y178E | L143E_K145T_Q179E_K228D | S121K_Q124R_T178R |
| kl-2 | 3971 | L143D_K145T | E124Q_T131R | L143R | Q124E_V133D_T180D |
| kl-2 | 3972 | A139W_L143D_K145T | E124Q_T131R | L143R | Q124E_V133D_L135W |
| kl-1 | 3974 | A125R_L143D_K145T | S122D_E124Q_T131R | L143R_K228D | S121K_Q124E_V133D |
| kl-2 | 3980 | L143D_K145T_F174G | T116F_E124Q_T131R_S176F | L143R_V190F | Q124E_V133D_L135A |
| kl-3 | 3987 | A139W_L143D_K145T | E124Q_T131R | L124R_S186K | V133G_L135W_S176D_T180E |
| kl-1 | 3997 | A125R_L143E_K145T_Q179E | S122D_E124Q_T131R | S188K_K228D | S121K_V1331_S176D_T178E |
| kl-8 | 4008 | A139W_L143K | K129T_V133D | L143E_K145T_Q179E | Q124R_L135W_T178R |
| kl-12 | 4010 | A139W_L143K | K129T_V133D | L124E_K145T_Q179E | S131R_V133G_L135W_S176R |
| kl-11 | 4022 | L143K_V190K | K129T_V133D_L135S | L124E_K145T | S131K_L135K |
| kl-8 | 4040 | S186R | K129T_S180E | L45P_L143E_K145T_Q179E | P44F_Q124K_T178R |
| kl-10 | 4320 | L143K_F174G | T116F_K129T_V133D_S176F | L143E_K145T_Q179E_V190F | Q124K_L135A_T178R |

TABLE 11B
Amino acid mutations in the Fab regions of H1, L1, H2 and L2
for K-K-derived K-L designs tested in SMCA format (Kabat numbering).

| | | H1 mutation | L1 mutation | H2 mutation | L2 mutation |
|---|---|---|---|---|---|
| Cluster | Design | (lambda) | (lambda) | (kappa) | (kappa) |
| kk-6 | 34 | Q39R | Q38E | Q39D | Q38R |
| kk-10 | 2188 | L124E | V133G_S176R | L124R | V133G_S176D |
| kk-8 | 2798 | — | — | L45P | P44F |
| kk-4 | 2816 | Q179K | S180E | L143E_K145T | Q124R_Q160K_T178R |
| kk-2 | 2826 | L143E_K145T | E124R | Q179K | Q124E_Q160E_T180E |
| kk-3 | 2827 | S186R | S180E | L143E_K145T_Q179E | Q124K_T178R |
| kk-1 | 2830 | L143E_K145T_Q179E | E124K_Y178R | S186R | T178E_T180E |

TABLE 12
Effect of DNA ratio titration on the percentage of antibody species, as assessed by LC-MS, of select wild-type systems

| | | H1:H2: | H1L1_H2L2 | | | | | | |
|---|---|---|---|---|---|---|---|---|---|
| | | L1:L2 | and | | | | | | |
| | | DNA | H1L2_H2L1 | H1L1_H1L1 | H1L1_H1L2 | H1L2_H1L2 | H2L1_H2L1 | H2L1_H2L2 | H2L2_H2L2 |
| System* | Tag | ratio | [symbol] | [symbol] | [symbol] | [symbol] | [symbol] | [symbol] | [symbol] |
| A | L2 FLAG | 10:20:24:46 | 40 | 0.2 | 0.3 | 0.1 | 0.5 | 0.4 | 0.6 |
| A | L2 FLAG | 8:22:17:53 | 36.3 | 0.1 | 0.2 | 0.2 | 0.2 | 0.5 | 1.4 |
| A | L2 FLAG | 8:22:13:57 | 33.8 | 0.1 | 0.3 | 0.1 | 0.3 | 0.4 | 1.9 |
| A | L2 FLAG | 10:20:35:35 | 30.4 | 0.3 | 0.1 | 0.1 | 0.5 | 0.2 | 0.3 |
| A | L2 FLAG | 15:15:35:35 | 19.5 | 1.8 | 1.8 | 0.9 | 0.1 | 0 | 0 |
| A | L2 FLAG | 8:22:35:35 | 16.7 | 0.2 | 0.1 | 0.1 | 0.7 | 0.2 | 0.2 |
| A | L2 FLAG | 10:20:46:24 | 11 | 0.8 | 0.1 | 0 | 0.3 | 0.1 | 0.1 |
| B^{†} | L2 FLAG | 8:22:53:17 | 3.2 | 0.5 | 0.1 | 0 | 1.3 | 0.1 | 0.1 |
| B^{†} | L2 FLAG | 20:10:37:33 | 36.8 | 0.5 | 1.1 | 1.6 | 0.1 | 0.2 | 0 |
| B^{†} | L2 FLAG | 20:10:24:46 | 34.7 | 0.1 | 0.5 | 1.6 | 0.2 | 0.1 | 0.1 |
| B^{†} | L2 FLAG | 20:10:46:24 | 33 | 1.1 | 0.8 | 0.6 | 0.1 | 0 | 0.1 |
| B^{†} | L2 FLAG | 22:8:35:35 | 31.1 | 0.8 | 1.5 | 2.2 | 0.1 | 0 | 0 |
| B^{†} | L2 FLAG | 20:10:35:35 | 29 | 0.3 | 2 | 2.1 | 0.1 | 0 | 0.1 |
| B^{†} | L2 FLAG | 22:8:53:17 | 26.8 | 2.4 | 2.1 | 0.8 | 0.1 | 0.1 | 0 |
| B^{†} | L2 FLAG | 15:15:35:35 | 43.7 | 0 | 0 | 0.5 | 0.2 | 0.5 | 0.3 |
| B^{†} | L2 FLAG | 22:8:17:53 | 13.3 | 0.0 | 1.0 | 5.7 | 0.3 | 0.1 | 0.0 |
| C^{†} | NO TAGS | 17:13:35:35 | 45.4 | 0.2 | 0.8 | 0.3 | 0 | 0 | 0 |
| C^{†} | NO TAGS | 15:15:35:35 | 47.8 | 0 | 0.2 | 0.1 | 0.2 | 0.2 | 0.1 |
| | | | H1:H2: | | | | | | |
| | | | L1:L2 | | | | | | |
| | | | DNA | H1L1_H2L1 | H1L2_H2L2 | H1L1 | H1L2 | H2L1 | H2L2 |
| | System* | Tag | ratio | [symbol] | [symbol] | [symbol] | [symbol] | [symbol] | [symbol] |
| | A | L2 FLAG | 10:20:24:46 | 20.3 | 26.2 | 0.8 | 0.5 | 3.9 | 6.1 |
| | A | L2 FLAG | 8:22:17:53 | 7.6 | 38.4 | 0.2 | 0.1 | 3.7 | 11.1 |
| | A | L2 FLAG | 8:22:13:57 | 5.1 | 39.9 | 0.3 | 0.1 | 3.8 | 13.9 |
| | A | L2 FLAG | 10:20:35:35 | 50.9 | 6.7 | 1.4 | 0 | 6 | 2.9 |
| | A | L2 FLAG | 15:15:35:35 | 7.5 | 18.2 | 32.9 | 16.5 | 0.2 | 0.5 |
| | A | L2 FLAG | 8:22:35:35 | 61.9 | 3.2 | 0.8 | 0.1 | 11.6 | 4.3 |
| | A | L2 FLAG | 10:20:46:24 | 78.6 | 1.3 | 4 | 0.1 | 3 | 0.4 |
| | B^{†} | L2 FLAG | 8:22:53:17 | 79.6 | 0.4 | 1.9 | 0 | 11.8 | 1 |
| | B^{†} | L2 FLAG | 20:10:37:33 | 19.6 | 22.4 | 8 | 9.5 | 0.3 | 0.1 |
| | B^{†} | L2 FLAG | 20:10:24:46 | 12.2 | 33.9 | 6.2 | 9.9 | 0.3 | 0.3 |
| | B^{†} | L2 FLAG | 20:10:46:24 | 40.6 | 9 | 9.3 | 5.2 | 0.2 | 0.1 |
| | B^{†} | L2 FLAG | 22:8:35:35 | 20.1 | 21.5 | 9.6 | 13 | 0 | 0 |
| | B^{†} | L2 FLAG | 20:10:35:35 | 14.5 | 20.4 | 13.5 | 17.5 | 0.3 | 0.1 |
| | B^{†} | L2 FLAG | 22:8:53:17 | 39.7 | 7.5 | 13.3 | 6.8 | 0.2 | 0.1 |
| | B^{†} | L2 FLAG | 15:15:35:35 | 22.6 | 24.8 | 0.4 | 0.4 | 2.9 | 3.7 |
| | B^{†} | L2 FLAG | 22:8:17:53 | 1.7 | 36.8 | 8.4 | 32.5 | 0.1 | 0.1 |
| | C^{†} | NO TAGS | 17:13:35:35 | 24.7 | 16.1 | 5.8 | 5.8 | 0.5 | 0.2 |
| | C^{†} | NO TAGS | 15:15:35:35 | 28.1 | 17.1 | 1.9 | 2.2 | 1.4 | 0.8 |

*System A: CAT-2200 H1/L1 & Pertuzumab H2/L2; System B: SGN-CD19a H1/L1 & CAT-2200 H2/L2; System C: SGN-CD19a H1/L1 & CR8071 H2/L2
^{†}Systems B^{†} and C^{†} for wild type controls differ from Systems B and C of designs in the orientation of H1L1 vs H2L2 warheads.

TABLE 13
LC-MS pairing data and post pA yields (mg/L) for wild type antibody constructs, and effect of tags on pairing

| | | | % | % | % H1L1 | | | | |
|---|---|---|---|---|---|---|---|---|---|
| | | Post | H1L1 | H2L2 | and % | | | | |
| | | pA | Pairing | Pairing | H2L2 | H1L1_H2L2 | H1L1_H2L2 | | |
| | | yield | (over | (over | Pairing | and | and | | |
| | | (mg/L | all H1 | all H2 | (over all | H1L2_H2L1** | H1L2_H2L1 | H1L1_H1L1 | H1L1_H1L2 |
| System* | Tag | A280) | species) | species) | species) | [symbol] | [symbol] | [symbol] | [symbol] |
| A | L2 | 41 | 57.4 | 79.9 | 70.6 | 47 | 36.5 | 0.3 | 0.3 |
| | FLAG | | | | | | | | |
| A | L1 HA | 45 | 19.1 | 93.1 | 61 | 16.9 | 14.3 | 0.2 | 0.3 |
| B^{†} | L2 | 44 | 64.1 | 64.8 | 60 | 34.4 | 26.3 | 0 | 0 |
| | FLAG | | | | | | | | |
| B^{†} | L1 | 58 | 37.6 | 92 | 71.2 | 32.1 | 23.5 | 0.2 | 0 |
| | FLAG | | | | | | | | |
| B^{†} | L2 HA | 72 | 54.2 | 70.1 | 66.7 | 35.1 | 17 | 0 | 0 |
| C^{†} | NO | 38 | 71.3 | 68.1 | 68.9 | 51.9 | 45.2 | 0 | 0 |
| | TAGS | | | | | | | | |
| C^{†} | L1 | 56 | 59.9 | 82.3 | 71.8 | 48.1 | 43.7 | 0.1 | 0 |
| | FLAG | | | | | | | | |
| C^{†} | L2 | 63 | 79.6 | 48.4 | 57.5 | 46.8 | 30.1 | 0 | 0 |
| | FLAG | | | | | | | | |
| | | H1L2_H1L2 | H2L1_H2L1 | H2L1_H2L2 | H2L2_H2L2 | H1L1_H2L1 | H1L2_H2L2 | H1L1 | H1L2 |
| System* | Tag | [symbol] | [symbol] | [symbol] | [symbol] | [symbol] | [symbol] | [symbol] | [symbol] |
| A | L2 | 0.1 | 0.2 | 0.3 | 0.6 | 14.6 | 23.9 | 1.7 | 1.9 |
| | FLAG | | | | | | | | |
| A | L1 HA | 0.3 | 0.2 | 0.1 | 0.4 | 1.4 | 67.8 | 0.1 | 0.7 |
| B^{†} | L2 | 0.5 | 0.3 | 0.6 | 1.1 | 15.9 | 24.8 | 0.4 | 0.4 |
| | FLAG | | | | | | | | |
| B^{†} | L1 | 0 | 0.1 | 0.4 | 2.1 | 3.1 | 43.7 | 1 | 2 |
| | FLAG | | | | | | | | |
| B^{†} | L2 HA | 0 | 1 | 2.2 | 3.1 | 5.8 | 19.2 | 0.3 | 0.2 |
| C^{†} | NO | 0 | 0.2 | 0.2 | 0.1 | 15.2 | 18.9 | 1.7 | 1.5 |
| | TAGS | | | | | | | | |
| C^{†} | L1 | 0.1 | 0.3 | 0.2 | 0.3 | 11.4 | 34.8 | 0.5 | 1.4 |
| | FLAG | | | | | | | | |
| C^{†} | L2 | 0.1 | 3.2 | 2.6 | 0.8 | 16.3 | 11.2 | 0.2 | 0.3 |
| | FLAG | | | | | | | | |
| | | | | | | | | H2L1 | H2L2 |
| | | | | | | System* | Tag | [symbol] | [symbol] |
| | | | | | | A | L2 | 4.3 | 12.3 |
| | | | | | | | FLAG | | |
| | | | | | | A | L1 HA | 3 | 11.1 |
| | | | | | | B^{†} | L2 | 4.9 | 14.1 |
| | | | | | | | FLAG | | |
| | | | | | | B^{†} | L1 | 3.1 | 20.9 |
| | | | | | | | FLAG | | |
| | | | | | | B^{†} | L2 HA | 18.5 | 32.7 |
| | | | | | | C^{†} | NO | 7.3 | 4.1 |
| | | | | | | | TAGS | | |
| | | | | | | C^{†} | L1 | 3.2 | 4 |
| | | | | | | | FLAG | | |
| | | | | | | C^{†} | L2 | 23.8 | 11.4 |
| | | | | | | | FLAG | | |

*System A: CAT-2200 H1/L1 & Pertuzumab H2/L2 (with 10:20:24:46 ratio H1:H2:L1:L2 DNA); System B: SGN-CD19a H1/L1 & CAT-2200 H2/L2 (with 15:15:35:35 ratio H1:H2:L1:L2 DNA); System C: SGN-CD19a H1/L1 & CR8071 H2/L2 (with 15:15:35:35 ratio H1:H2:L1:L2 DNA)
Multiple LCMS SMCA experiments were performed for some constructs. In those cases, the mean pairing values and median amounts of each SMCA species are reported.
**considering full Ab species only
^{†}Systems B^{†} and C^{†} for wild type controls differ from Systems B and C of designs in the orientation of H1L1 vs H2L2 warheads.

TABLE 14
LC-MS pairing data and post pA yields (mg/L) for K-L designs (total pairing)

| | | | | | | | Change in | | Change in | | Change in | | | |
|---|---|---|---|---|---|---|---|---|---|---|---|---|---|---|
| | | | | | Change in | | % H2L2 | | % H1L1 | | H1L1_H2L2 | | | |
| | | | | | % H1L1 | | Pairing | % | and | | and | | | |
| | | | | | Pairing | | with | H1L1 | % H2L2 | H1L1_H2L2 | H1L2_H2L1** | H1L1_H2L2 | | |
| | | | Post pA yield | % | with | % | respect | and % | Pairing with | and | with | and | | |
| | | | (mg per L | H1L1 | respect to | H2L2 | to wild | H2L2 | respect to | H1L2_H2L1** | respect to | H1L2_H2L1 | H1L1_H1L1 | H1L1_H1L2 |
| Design^{§} | Cluster | System* | expression) | Pairing | wild type | Pairing | type | Pairing | wild type | [symbol] | wild type | [symbol] | [symbol] | [symbol] |

Transferable in 3/3 systems:

| | | | | | | | | | | | | | | |
|---|---|---|---|---|---|---|---|---|---|---|---|---|---|---|
| 2892 | kl-6 | A | 28 | 81.3 | 62.2 | 98.3 | 5.2 | 94.1 | 33.1 | 71.8 | 54.9 | 39.1 | 0.1 | 0.2 |
| | | B | 34 | 84.7 | 14.6 | 94.3 | 40.1 | 88.7 | 22 | 84.2 | 49.1 | 63.4 | 0.6 | 0.1 |
| | | C | 32 | 41.5 | −6.9 | 99.6 | 20 | 69.9 | 12.4 | 41 | −5.8 | 34.3 | 0.1 | 0.6 |
| 2901 | kl-6 | A | 20 | 94.3 | 75.2 | 97.9 | 4.8 | 96.6 | 35.6 | 87 | 70.2 | 64.1 | 0.2 | 0.3 |
| | | B | 18 | 85.7 | 15.6 | 94.6 | 40.4 | 88.8 | 22.1 | 79.2 | 44.1 | 48.2 | 1.6 | 0.3 |
| | | C | 19 | 62.6 | 14.2 | 99.5 | 19.9 | 78.7 | 21.2 | 60.6 | 13.8 | 46.6 | 1.4 | 1.2 |
| 2979 | kl-4 | A | 20 | 99.4 | 47.6^{‡} | 98.3 | 27.2^{‡} | 98.8 | 31.5^{‡} | 95.7 | 63.8^{‡} | 83.8 | 1.5 | 0 |
| | | B | 30 | 98 | 6 | 77.3 | 39.7 | 86.7 | 15.5 | 72.4 | 40.3 | 63.7 | 0.2 | 0.1 |
| | | C | 14 | 94.5 | 12.2 | 99.5 | 39.6 | 98.5 | 26.7 | 79.9 | 31.8 | 34.3 | 0 | 0.7 |
| 3008 | kl-3 | A | 27 | 98.8 | 79.7 | 98.3 | 5.2 | 98.5 | 37.5 | 96.7 | 79.8 | 75.9 | 0 | 0.1 |
| | | B | 26 | 98.3 | 28.2 | 98.8 | 44.6 | 98.4 | 31.7 | 93.2 | 58.1 | 57 | 2.1 | 0 |
| | | C | 17 | 84.7 | 36.3 | 99.6 | 20 | 90.1 | 32.6 | 78.2 | 31.4 | 56.5 | 2.9 | 3.8 |
| 3018 | kl-3 | A | 23 | 97.1 | 78 | 98.7 | 5.6 | 98 | 37 | 95.3 | 78.4 | 72.4 | 0 | 0 |
| | | B | 33 | 96 | 25.9 | 98.6 | 44.4 | 96.5 | 29.8 | 77.9 | 42.8 | 33.9 | 7.1 | 0.1 |
| | | C | 18 | 79.7 | 11.6 | 99.5 | 28.2 | 84.3 | 15.4 | 70 | 18.1 | 34.3 | 3.4 | 0.8 |
| 3025 | kl-2 | A | 35 | 83.6 | 64.5 | 99.1 | 6 | 93.9 | 33 | 72.7 | 55.9 | 54.9 | 0.1 | 0 |
| | | B | 30 | 80.2 | 10.1 | 87.9 | 33.7 | 85.5 | 18.8 | 71.4 | 36.3 | 44.9 | 0 | 0 |
| | | C | 37 | 43.9 | −4.5 | 99.7 | 20.1 | 70.1 | 12.6 | 41.1 | −5.7 | 40.4 | 1.8 | 1.4 |
| 3102 | kl-12 | A | 28 | 96.5 | 44.7^{‡} | 97.6 | 26.5^{‡} | 97.1 | 29.8^{‡} | 93.1 | 61.2^{‡} | 81.3 | 0.1 | 0 |
| | | B | 33 | 96.6 | 4.6 | 92.8 | 55.2 | 94.4 | 23.2 | 86.9 | 54.8 | 74.7 | 0.2 | 0.1 |
| | | C | 27 | 88.7 | 6.4 | 99.8 | 39.9 | 98.1 | 26.3 | 65.2 | 17.1 | 27.1 | 0.1 | 0.4 |
| 3109 | kl-8 | A | 16 | 99.7 | 47.9^{‡} | 97.4 | 26.3^{‡} | 98.6 | 31.3^{‡} | 96.4 | 64.5^{‡} | 81.3 | 0.8 | 0.1 |
| | | B | 35 | 99.1 | 29^{‡} | 97.1 | 42.9^{‡} | 98 | 31.3^{‡} | 94.1 | 59^{‡} | 78.4 | 0.3 | 0.1 |
| | | C | 21 | 84 | 15.9^{‡} | 99.6 | 28.3^{‡} | 97.9 | 29^{‡} | 66.8 | 18.7^{‡} | 17.6 | 0.1 | 0.3 |
| 3113 | kl-12 | A | 33 | 97 | 45.2^{‡} | 98.4 | 27.3^{‡} | 97.8 | 30.5^{‡} | 93.8 | 61.9^{‡} | 74.7 | 0.2 | 0 |
| | | B | 43 | 98.9 | 6.9 | 90.9 | 53.3 | 94.3 | 23.1 | 85.6 | 53.5 | 72.8 | 0.3 | 0.2 |
| | | C | 28 | 76.7 | −5.6 | 99.6 | 39.7 | 95.2 | 23.4 | 64 | 15.9 | 28.3 | 0 | 0.6 |
| 3122 | kl-5 | A | 16 | 85.9 | 34.1^{‡} | 23.4 | −47.7^{‡} | 85.8 | 18.5^{‡} | 0 | −31.9^{‡} | 0 | 10.4 | 2.5 |
| | | B | 38 | 96.9 | 4.9 | 89.7 | 52.1 | 92.9 | 21.7 | 84.7 | 52.6 | 72.7 | 0.2 | 0.2 |
| | | C | 45 | 65.8 | −16.5 | 99.8 | 39.9 | 91.1 | 19.3 | 61.5 | 13.4 | 32.6 | 0.1 | 0.4 |
| 3878 | kl-8 | A | 21 | 99.7 | 47.9^{‡} | 89.7 | 18.6^{‡} | 94.6 | 27.3^{‡} | 87.6 | 55.7^{‡} | 78.1 | 0.6 | 0 |
| | | B | 31 | 97.7 | 5.7 | 28.7 | −8.9 | 71.8 | 0.6 | 24.2 | −7.9 | 17.3 | 3.6 | 0.7 |
| | | C | 30 | 99.3 | 17 | 67.4 | 7.5 | 88.2 | 16.4 | 61.3 | 13.2 | 42.6 | 6 | 0.6 |
| 3890 | kl-9 | A | 29 | 99.8 | 48^{‡} | 87.5 | 16.4^{‡} | 93.3 | 26^{‡} | 85.1 | 53.2^{‡} | 70.7 | 1.7 | 0 |
| | | B | 24 | 99.7 | 29.6^{‡} | 51.3 | −2.9^{‡} | 73.7 | 7^{‡} | 47.7 | 12.6^{‡} | 41.4 | 1.1 | 0 |
| | | C | 27 | 98.4 | 30.3 | 88.5 | 17.2 | 92.8 | 23.9 | 86.3 | 34.4 | 72.3 | 0.7 | 0.3 |
| 3898 | kl-8 | A | 15 | 98.7 | 46.9^{‡} | 98.7 | 27.6^{‡} | 98.7 | 31.4^{‡} | 93.9 | 62^{‡} | 81.4 | 2.3 | 0 |
| | | B | 21 | 97.3 | 5.3 | 74.5 | 36.9 | 85.9 | 14.7 | 71.1 | 39 | 68.7 | 0.5 | 0.3 |
| | | C | 11 | 97.4 | 15.1 | 95.2 | 35.3 | 96.3 | 24.5 | 91.4 | 43.3 | 87.4 | 0.4 | 0.8 |
| 3931 | kl-7 | A | 30 | 92.2 | 73.1 | 94.9 | 1.8 | 93.6 | 32.6 | 83.9 | 67.1 | 70.7 | 0.5 | 0.2 |
| | | B | 39 | 85.5 | 15.4 | 83.6 | 29.4 | 85 | 18.3 | 60.7 | 25.6 | 35.4 | 6.8 | 1.7 |
| | | C | 40 | 57.1 | 8.7 | 99.5 | 19.9 | 68.6 | 11.1 | 45.7 | −1.1 | 27.3 | 4.6 | 3.5 |
| 3947 | kl-4 | A | 16 | 99.6 | 47.8^{‡} | 92.8 | 21.7^{‡} | 96.3 | 29^{‡} | 89.1 | 57.2^{‡} | 73.2 | 2.3 | 0 |
| | | B | 24 | 98.8 | 28.7^{‡} | 70.3 | 16.1^{‡} | 81.7 | 15^{‡} | 66.1 | 31^{‡} | 53.4 | 0.2 | 0.3 |
| | | C | 11 | 91.3 | 23.2 | 94.5 | 23.2 | 94 | 25.1 | 65.7 | 13.8 | 23.4 | 0.2 | 0.2 |
| 3951 | kl-4 | A | 14 | 98.7 | 46.9^{‡} | 89.5 | 18.4^{‡} | 93.2 | 25.9^{‡} | 83.5 | 51.6^{‡} | 63.3 | 0.2 | 0 |
| | | B | 23 | 99.8 | 29.7^{‡} | 77.5 | 23.3^{‡} | 85.6 | 18.9^{‡} | 72.8 | 37.7^{‡} | 52.5 | 0.1 | 0 |
| | | C | 23 | 97.5 | 29.4 | 97.1 | 25.8 | 97.3 | 28.4 | 92.2 | 40.3 | 73.6 | 0 | 0.2 |
| 3954 | kl-7 | A | 35 | 99.1 | 47.3^{‡} | 97.7 | 26.6^{‡} | 98.3 | 31^{‡} | 92.7 | 60.8^{‡} | 71.9 | 1 | 0 |
| | | B | 38 | 98.4 | 28.3^{‡} | 61.8 | 7.6^{‡} | 88.6 | 21.9^{‡} | 46 | 10.9^{‡} | 27.3 | 12.9 | 0.1 |
| | | C | 39 | 89 | 40.6 | 98.7 | 19.1 | 92.4 | 34.9 | 78.1 | 31.3 | 55.4 | 8.9 | l.l |
| 3957 | kl-7 | A | 32 | 90.2 | 71.1 | 90.6 | −2.5 | 90.4 | 29.5 | 78.8 | 61.9 | 66.8 | 0.6 | 0.1 |
| | | B | 34 | 99 | 28.9 | 85.4 | 31.2 | 95.6 | 28.9 | 70.9 | 35.8 | 36.5 | 7.9 | 0.1 |
| | | C | 41 | 87.2 | 38.8 | 99.7 | 20.1 | 90.8 | 33.3 | 75.5 | 28.7 | 45.7 | 8 | 0.9 |
| 3967 | kl-9 | A | 12 | 100 | 42.6 | 99.9 | 20 | 99.9 | 29.3 | 97.2 | 50.2 | 68.3 | 1.8 | 0 |
| | | B | 18 | 97.1 | 27^{‡} | 98.6 | 44.4^{‡} | 98 | 31.3^{‡} | 91.8 | 56.7^{‡} | 69.4 | 0.3 | 0.1 |
| | | C | 14 | 96.7 | 28.6 | 100 | 28.7 | 98.6 | 29.7 | 95.2 | 43.3 | 80 | 0.8 | 1.3 |
| 3971 | kl-2 | A | 27 | 98.9 | 79.8 | 99.1 | 6 | 99.1 | 38.1 | 87.4 | 70.6 | 45.6 | 0.2 | 0 |
| | | B | 27 | 97.9 | 27.8 | 99.3 | 45.1 | 98.9 | 32.2 | 93.2 | 58.1 | 62.3 | 0 | 0 |
| | | C | 30 | 89 | 40.6 | 99.8 | 20.2 | 92.5 | 35 | 82.6 | 35.8 | 54.2 | 3.9 | 0.7 |
| 3972 | kl-2 | A | 25 | 99.9 | 48.1^{‡} | 99.5 | 28.4^{‡} | 99.7 | 32.4^{‡} | 96.9 | 65^{‡} | 79.4 | 1.2 | 0 |
| | | B | 36 | 98.1 | 28^{‡} | 99.7 | 45.5^{‡} | 98.5 | 31.8^{‡} | 84 | 48.9^{‡} | 46.5 | 8.1 | 0.1 |
| | | C | 38 | 92.8 | 44.4 | 99.8 | 20.2 | 95.2 | 37.7 | 81.3 | 34.5 | 57.4 | 5.7 | 1.1 |
| 3974 | kl-1 | A | 22 | 91.8 | 72.7 | 94.7 | 1.6 | 93.5 | 32.5 | 83.7 | 66.8 | 62.3 | 0.4 | 4.4 |
| | | B | 38 | 97.6 | 27.5 | 92.6 | 38.4 | 96.5 | 29.8 | 72.9 | 37.8 | 34 | 8.3 | 0.3 |
| | | C | 36 | 59.9 | 11.5 | 99.8 | 20.2 | 71 | 13.5 | 49.5 | 2.7 | 31.4 | 5.6 | 4.5 |
| 3980^{†} | kl-2 | A | 26 | 99.7 | 47.9^{‡} | 74.8 | 3.7^{‡} | 86.9 | 19.6^{‡} | 71.4 | 39.5^{‡} | 59 | 1.9 | 0.1 |
| | | B | 37 | 99.8 | 29.7^{‡} | 82.6 | 28.4^{‡} | 95.1 | 28.4^{‡} | 69.6 | 34.5^{‡} | 39.6 | 8.3 | 0 |
| 3987 | kl-3 | A | 25 | 99.7 | 47.9^{‡} | 83 | 11.9^{‡} | 93.9 | 26.6^{‡} | 80.7 | 48.8^{‡} | 51.6 | 2.7 | 0 |
| | | B | 27 | 97.5 | 27.4^{‡} | 98.1 | 43.9^{‡} | 97.6 | 30.9^{‡} | 66.4 | 31.3^{‡} | 24.8 | 11.1 | 0.3 |
| | | C | 21 | 99.2 | 50.8 | 99.3 | 19.7 | 99.2 | 41.7 | 78.9 | 32.1 | 37.4 | 9.2 | 0.4 |
| 3997 | kl-l | A | 14 | 99.8 | 48^{‡} | 90.7 | 19.6^{‡} | 94.9 | 27.6^{‡} | 85.9 | 54^{‡} | 69 | 0.6 | 0 |
| | | B | 25 | 98.8 | 28.7^{‡} | 53 | −1.2^{‡} | 90.6 | 23.9^{‡} | 38 | 2.9^{‡} | 16 | 12.2 | 0.2 |
| | | C | 6 | 96 | 27.9 | 99.9 | 28.6 | 96 | 27.1 | 2.9 | −49 | 0.4 | 11.2 | 0.7 |
| 4008 | kl-8 | A | 22 | 100 | 42.6 | 99.3 | 19.4 | 99.7 | 29.1 | 96.5 | 49.5 | 81.6 | 1.2 | 0 |
| | | B | 30 | 99.6 | 7.6 | 70.9 | 33.3 | 84.6 | 13.4 | 68.7 | 36.6 | 60.2 | 0.6 | 0 |
| | | C | 26 | 99.6 | 17.3 | 98.3 | 38.4 | 98.9 | 27.1 | 96.8 | 48.7 | 85.5 | 0.1 | 0.3 |
| 4010 | kl-12 | A | 29 | 99.9 | 42.5 | 98.9 | 19 | 99.5 | 28.9 | 98.1 | 51.1 | 76.7 | 0.2 | 0 |
| | | B | 27 | 99.5 | 7.5 | 71.5 | 33.9 | 84.8 | 13.6 | 67.1 | 35 | 59.9 | 0.5 | 0.1 |
| | | C | 23 | 99.3 | 17 | 98.6 | 38.7 | 98.9 | 27.1 | 96.7 | 48.6 | 83.6 | 0.2 | 0.4 |
| 4040 | kl-8 | A | 29 | 99.8 | 48^{‡} | 97.7 | 26.6^{‡} | 98.7 | 31.4^{‡} | 94.6 | 62.7^{‡} | 85.8 | 1.2 | 0 |
| | | B | 41 | 99.2 | 29.1^{‡} | 75.8 | 21.6^{‡} | 86 | 19.3^{‡} | 73.7 | 38.6^{‡} | 62.7 | 0.6 | 0 |
| | | C | 19 | 98.7 | 30.6 | 98.6 | 27.3 | 98.7 | 29.8 | 81.8 | 29.9 | 51.2 | 1.2 | 0.1 |
| 4320 | kl-10 | A | 14 | 99.7 | 47.9^{‡} | 91.4 | 20.3^{‡} | 95.4 | 28.1^{‡} | 89.1 | 57.2^{‡} | 77.7 | 0.6 | 0 |
| | | B | 35 | 99.8 | 7.8 | 55.5 | 17.9 | 73.4 | 2.2 | 47.9 | 15.8 | 38.9 | 0.3 | 0.1 |
| | | C | 22 | 96.6 | 14.3 | 81.4 | 21.5 | 89 | 17.2 | 77.1 | 29 | 75 | 0.6 | 3.2 |

Transferable in 2/3 systems:

| | | | | | | | | | | | | | | |
|---|---|---|---|---|---|---|---|---|---|---|---|---|---|---|
| 2994 | kl-3 | A | 35 | 78.3 | 59.2 | 98.9 | 5.8 | 92 | 31 | 70.1 | 53.2 | 50 | 0.7 | 0 |
| | | B | 42 | 84.8 | 14.7 | 99.2 | 45 | 90.9 | 24.2 | 80.9 | 45.8 | 64.6 | 3.5 | 0.3 |
| | | C | 33 | 26.3 | −22.1 | 99.7 | 20.1 | 57.4 | −41.1 | 25.5 | −21.3 | 19.9 | 0.3 | 1.6 |
| 3041 | kl-3 | A | 8 | 66.7 | 47.6 | 23.5 | −69.6 | 66.6 | 5.6 | 0 | −16.9 | 0 | 5.4 | 2.1 |
| | | B | 29 | 75.6 | 5.5 | 99.6 | 45.4 | 92.2 | 25.5 | 71 | 35.9 | 46.3 | 0.1 | 0 |
| | | C | 26 | 27.2 | −21.2 | 99.9 | 20.3 | 54.4 | −3.1 | 29 | −17.8 | 20.9 | 0.8 | 1.9 |
| 3910 | kl-10 | A | 20 | 99.6 | 47.8^{‡} | 57 | −14.1^{‡} | 79.1 | 11.8^{‡} | 54.1 | 22.2^{‡} | 43.9 | 1.8 | 0 |
| | | B | 41 | 99.6 | 7.6 | 39.7 | 2.1 | 66.9 | −4.3 | 34 | 1.9 | 29.9 | 0.5 | 0.1 |
| | | C | 52 | 99.3 | 17 | 81 | 21.1 | 89.5 | 17.7 | 78.7 | 30.6 | 73.2 | 0.3 | 0.2 |

Transferable in 1/3 systems:

| 4022 | kl-11 | A | 10 | 70.4 | 18.6^{‡} | 90.4 | 19.3^{‡} | 80.6 | 13.3^{‡} | 62.9 | 31^{‡} | 57.3 | 1.3 | 0.2 |
|---|---|---|---|---|---|---|---|---|---|---|---|---|---|---|
| | | B | 20 | 35.4 | −34.7^{‡} | 90.4 | 36.2^{‡} | 61.3 | −5.4^{‡} | 40.6 | 5.5^{‡} | 27 | 0.4 | 0.5 |
| | | C | 21 | 10 | −38.4 | 87.6 | 8 | 48 | −9.5 | 7.7 | −39.1 | 7 | 0.5 | 1 |
| | | | | | H1L2_H1L2 | H2L1_H2L1 | H2L1_H2L2 | H2L2_H2L2 | H1L1_H2L1 | H1L2_H2L2 | H1L1 | H1L2 | H2L1 | H2L2 |
| | | Design^{§} | Cluster | System* | [symbol] | [symbol] | [symbol] | [symbol] | [symbol] | [symbol] | [symbol] | [symbol] | [symbol] | [symbol] |
| | | Transferable in 3/3 systems: | | | | | | | | | | | | |
| | | 2892 | kl-6 | A | 0.2 | 0.3 | 1 | 5 | 0.2 | 8.3 | 0.4 | 0.2 | 0.3 | 44.6 |
| | | | | B | 0.5 | 0.3 | 0.2 | 0.1 | 3.2 | 7 | 15.8 | 4.9 | 0.3 | 3.7 |
| | | | | C | 0.8 | 0 | 0 | 0.1 | 0.2 | 47.5 | 3.6 | 5.1 | 0 | 7.6 |
| | | 2901 | kl-6 | A | 0.1 | 0.3 | 1.5 | 3.5 | 0.4 | 3.4 | 2.3 | 0.2 | 0.1 | 23.7 |
| | | | | B | 0.3 | 0.3 | 0.2 | 0.2 | 2.2 | 7.7 | 29 | 5 | 0.4 | 4.7 |
| | | | | C | 1.4 | 0.1 | 0 | 0.5 | 0 | 25.7 | 10 | 6.3 | 0.1 | 6.7 |
| | | 2979 | kl-4 | A | 0.2 | 0.9 | 0 | 1 | 0 | 0.2 | 5.4 | 0 | 0 | 7.1 |
| | | | | B | 0.8 | 0.1 | 0 | 0.8 | 22.1 | 0 | 1.2 | 0 | 1.2 | 9.6 |
| | | | | C | 0 | 0.1 | 0 | 6 | 0.5 | 1.3 | 0 | 0 | 0.1 | 56.9 |
| | | 3008 | kl-3 | A | 0.2 | 0.1 | 0.3 | 0.2 | 1.3 | 0.5 | 2.1 | 0.1 | 0.1 | 19.3 |
| | | | | B | 0.2 | 0.1 | 0 | 0.1 | 0.2 | 1.4 | 35.8 | 0.3 | 0.1 | 2.7 |
| | | | | C | 0.5 | 0 | 0.1 | 0.3 | 0.1 | 8.1 | 20.8 | 3.4 | 0 | 3.5 |
| | | 3018 | kl-3 | A | 0.1 | 0.1 | 0 | 1 | 0.6 | 1.7 | 3.1 | 0.2 | 0.4 | 20.3 |
| | | | | B | 0.2 | 0.1 | 0 | 0 | 0.2 | 1.8 | 52.9 | 2.1 | 0.1 | 1.4 |
| | | | | C | 0.3 | 0 | 0 | 0 | 0.2 | 10.1 | 40.4 | 9.8 | 0 | 0.8 |
| | | 3025 | kl-2 | A | 0 | 0.1 | 0.4 | 8.9 | 0.4 | 10.7 | 0.1 | 0.1 | 0.1 | 24.1 |
| | | | | B | 0.1 | 0.1 | 0.1 | 2 | 3.6 | 12 | 0.6 | 0 | 6.4 | 30.1 |
| | | | | C | 2.3 | 0.1 | 0 | 0.2 | 0.1 | 52.1 | 0.6 | 0.7 | 0 | 0.3 |
| | | 3102 | kl-12 | A | 0.5 | 1.2 | 0.2 | 1.8 | 0 | 2.1 | 2.8 | 0 | 0 | 9.9 |
| | | | | B | 1.3 | 0.1 | 0 | 2 | 7.4 | 0 | 0.1 | 0 | 0.3 | 13.7 |
| | | | | C | 0 | 0.1 | 0 | 11 | 0.1 | 2.9 | 0 | 0.1 | 0 | 58.2 |
| | | 3109 | kl-8 | A | 0 | 0.5 | 1.2 | 0.3 | 0 | 0.2 | 10.7 | 0 | 0.1 | 4.8 |
| | | | | B | 0 | 0 | 0 | 1.1 | 2.8 | 0.6 | 0.2 | 0 | 0.2 | 16.2 |
| | | | | C | 0.1 | 0.1 | 0 | 5.6 | 0 | 2.6 | 0.1 | 0.2 | 0.2 | 73.1 |
| | | 3113 | kl-12 | A | 0 | 0.9 | 0 | 1.3 | 0.1 | 2.4 | 2.1 | 0 | 0 | 18.2 |
| | | | | B | 0.1 | 0 | 0 | 1.7 | 9.5 | 0.4 | 1.1 | 0 | 0.4 | 13.4 |
| | | | | C | 0.1 | 0.2 | 0 | 7.8 | 0.3 | 7 | 0.1 | 0.6 | 0 | 55 |
| | | 3122 | kl-5 | A | 0.5 | 0 | 0 | 0 | 0.2 | 0 | 74 | 12.3 | 0 | 0 |
| | | | | B | 0.5 | 0.1 | 0 | 0.8 | 10.2 | 1 | 1.6 | 0.3 | 0.5 | 11.8 |
| | | | | C | 0.5 | 0.1 | 0 | 3.4 | 0.2 | 15.9 | 0.2 | 0.1 | 0 | 46.7 |
| | | 3878 | kl-8 | A | 0 | 0.2 | 0.3 | 1 | 8.7 | 0.2 | 4.7 | 0 | 0.6 | 5.5 |
| | | | | B | 1 | 0.9 | 0.1 | 0.2 | 47.8 | 0.1 | 24.5 | 0 | 2 | 1.9 |
| | | | | C | 0.1 | 0 | 0.2 | 0.1 | 19.7 | 0.1 | 27.3 | 0 | 1.4 | 1.9 |
| | | 3890 | kl-9 | A | 0 | 0 | 0 | 1.2 | 9.2 | 0.1 | 5.4 | 0 | 1.9 | 9.5 |
| | | | | B | 0 | 0.2 | 0.1 | 0.4 | 43.4 | 0.1 | 2.7 | 0 | 4.2 | 6.4 |
| | | | | C | 0.3 | 0 | 0.2 | 0.9 | 8.5 | 0.5 | 2.1 | 0 | 2 | 12.1 |
| | | 3898 | kl-8 | A | 0 | 0.6 | 0 | 1 | 0 | 1.3 | 9.1 | 0 | 0 | 4.3 |
| | | | | B | 1 | 0 | 0 | 0.8 | 25 | 0.2 | 1.1 | 0 | 0.2 | 2.1 |
| | | | | C | 0 | 0 | 0.1 | 0.6 | 4.8 | 1.6 | 1 | 0.1 | 0 | 3.2 |
| | | 3931 | kl-7 | A | 0 | 0.2 | 1.7 | 1.5 | 3 | 6.5 | 7.2 | 0.4 | 0.1 | 8 |
| | | | | B | 0.2 | 0.2 | 0 | 0 | 7.9 | 6 | 32.6 | 6.4 | 0.3 | 2.3 |
| | | | | C | 2.2 | 0 | 0 | 0 | 0.1 | 22 | 21.6 | 16.3 | 0.1 | 2.2 |
| | | 3947 | kl-4 | A | 0 | 0.1 | 0.1 | 1.2 | 5 | 0.4 | 9.5 | 0 | 0.9 | 7.4 |
| | | | | B | 0 | 0.3 | 0.5 | 1.9 | 23.6 | 0.6 | 0.7 | 0 | 5.5 | 13 |
| | | | | C | 0.1 | 0 | 0.1 | 8 | 1.7 | 2 | 2.4 | 0.3 | 3.7 | 57.9 |
| | | 3951 | kl-4 | A | 0.3 | 1.7 | 0 | 3 | 6.8 | 0.3 | 4.2 | 0 | 1.1 | 18.8 |
| | | | | B | 0 | 0.1 | 0.2 | 1.5 | 17.7 | 0.1 | 0.9 | 0 | 5.3 | 21.6 |
| | | | | C | 0 | 0 | 0 | 2.6 | 1.6 | 1.8 | 0.5 | 0 | 0.9 | 18.8 |
| | | 3954 | kl-7 | A | 0 | 0.5 | 0 | 2.1 | 1.2 | 0.8 | 4.2 | 0 | 0.2 | 18 |
| | | | | B | 0.1 | 0 | 0 | 0.1 | 17.6 | 1.1 | 36.6 | 0.4 | 1.4 | 2.2 |
| | | | | C | 0.1 | 0 | 0 | 0.2 | 0.6 | 4.6 | 20.5 | 4.2 | 0.1 | 4.3 |
| | | 3957 | kl-7 | A | 0 | 0.1 | 0.1 | 0.4 | 7.9 | 8.6 | 8.9 | 0.7 | 0.4 | 5.3 |
| | | | | B | 0.1 | 0.1 | 0 | 0 | 6.4 | 0.3 | 44.4 | 0.4 | 0.4 | 3.3 |
| | | | | C | 0.2 | 0 | 0 | 0.2 | 0 | 5.5 | 31.2 | 5.8 | 0.1 | 2.4 |
| | | 3967 | kl-9 | A | 0 | 0 | 0 | 0 | 0 | 0 | 21.8 | 0 | 0 | 7.9 |
| | | | | B | 0 | 0 | 0.1 | 2 | 1.7 | 2.1 | 1.1 | 0 | 0 | 23.3 |
| | | | | C | 0 | 0 | 0 | 0.7 | 0 | 1.3 | 0.6 | 0.2 | 0 | 15.3 |
| | | 3971 | kl-2 | A | 0 | 0.1 | 0.3 | 5.1 | 0.7 | 0.1 | 0.2 | 0.2 | 0.1 | 47.4 |
| | | | | B | 0.2 | 0.1 | 0 | 3 | 0.3 | 1 | 0.7 | 0 | 0.2 | 32.2 |
| | | | | C | 0.2 | 0 | 0 | 0.1 | 0.1 | 6.5 | 28.4 | 3.6 | 0 | 2.3 |
| | | 3972 | kl-2 | A | 0 | 0.1 | 0 | 0.9 | 0.2 | 0.1 | 12.1 | 0 | 0 | 6 |
| | | | | B | 0.1 | 0 | 0 | 0.3 | 0.1 | 0.2 | 40 | 1.1 | 0 | 3.5 |
| | | | | C | 0 | 0 | 0 | 0.2 | 0.1 | 6 | 25.2 | 1.1 | 0 | 3.1 |
| | | 3974 | kl-1 | A | 0 | 0.3 | 0.4 | 1.9 | 2.8 | 2 | 4.1 | 0.3 | 1.1 | 20 |
| | | | | B | 0.2 | 0.2 | 0 | 0 | 2.7 | 1 | 49.3 | 1.1 | 0.1 | 3 |
| | | | | C | 1.8 | 0 | 0 | 0.1 | 0 | 20.1 | 19.7 | 14.9 | 0 | 1.9 |
| | | 3980^{†} | kl-2 | A | 0 | 1.1 | 0.1 | 1.4 | 18.9 | 0.1 | 7.8 | 0.1 | 2.3 | 7.2 |
| | | | | B | 0 | 0.1 | 0.1 | 0.2 | 8.6 | 0.1 | 40.2 | 0.1 | 0.3 | 2.4 |
| | | 3987 | kl-3 | A | 0.1 | 1.2 | 0.4 | 0.1 | 7.7 | 0.1 | 33 | 0.1 | 0.6 | 2.3 |
| | | | | B | 0.2 | 0 | 0 | 0.1 | 0.4 | 0.4 | 60.4 | 1.6 | 0 | 0.7 |
| | | | | C | 0 | 0 | 0 | 0.1 | 0 | 0.2 | 51.8 | 0.3 | 0.1 | 0.4 |
| | | 3997 | kl-l | A | 0.1 | 0.4 | 0 | 1.1 | 9.1 | 0.1 | 6.6 | 0 | 0 | 13 |
| | | | | B | 0.1 | 0 | 0 | 0 | 13.6 | 0 | 54 | 0.8 | 1.6 | 1.4 |
| | | | | C | 0.1 | 0 | 0 | 0 | 0 | 0.4 | 83.8 | 3.3 | 0 | 0.1 |
| | | 4008 | kl-8 | A | 0 | 0 | 0 | 1 | 0.6 | 0 | 8.7 | 0 | 0 | 6.9 |
| | | | | B | 0.1 | 0.1 | 0.1 | 0.7 | 25.7 | 0.1 | 4.1 | 0 | 2.2 | 6.1 |
| | | | | C | 0 | 0 | 0 | 0.7 | 1.7 | 0 | 1.3 | 0 | 0.1 | 10.3 |
| | | 4010 | kl-12 | A | 0 | 0.1 | 0.1 | 0.4 | 0.7 | 0 | 15.3 | 0 | 0 | 6.6 |
| | | | | B | 0.1 | 0.1 | 0.3 | 0.4 | 27.7 | 0.1 | 3 | 0 | 0.9 | 6.9 |
| | | | | C | 0 | 0 | 0 | 0.8 | 1.4 | 0 | 0.8 | 0 | 0.1 | 12.6 |
| | | 4040 | kl-8 | A | 0 | 0.9 | 0 | 2 | 0.7 | 0.1 | 3.7 | 0 | 0 | 5.5 |
| | | | | B | 0 | 0.1 | 0.1 | 0.6 | 20.4 | 0.6 | 1.3 | 0 | 3.3 | 10.3 |
| | | | | C | 0 | 0 | 0.3 | 8.2 | 1 | 0.5 | 0.5 | 0 | 0.3 | 36.7 |
| | | 4320 | kl-10 | A | 0 | 0.1 | 0.1 | 1.3 | 7.3 | 0.2 | 5 | 0 | 0.7 | 7.1 |
| | | | | B | 0 | 1 | 0.3 | 0.7 | 39.8 | 0.1 | 0.8 | 0 | 5.4 | 12.6 |
| | | | | C | 0 | 0.1 | 0.7 | 0.9 | 16.8 | 0.1 | 0.2 | 0.1 | 0.5 | 2 |
| | | Transferable in 2/3 systems: | | | | | | | | | | | | |
| | | 2994 | kl-3 | A | 0 | 0.2 | 1 | 5.1 | 0.1 | 14.2 | 0.6 | 0.1 | 0 | 28 |
| | | | | B | 0.3 | 0.2 | 0 | 0.1 | 0.2 | 10.6 | 12.9 | 3 | 0 | 4.2 |
| | | | | C | 3.1 | 0 | 0 | 0.1 | 0 | 53.1 | 4.1 | 12 | 0.1 | 5.7 |
| | | 3041 | kl-3 | A | 1.4 | 0 | 0 | 0 | 0 | 0 | 60.2 | 30.7 | 0.2 | 0.1 |
| | | | | B | 0.3 | 0.2 | 0 | 3.8 | 0.1 | 14.5 | 0.1 | 0 | 0.1 | 34.6 |
| | | | | C | 4 | 0 | 0 | 0.1 | 0 | 44.3 | 4.8 | 18.5 | 0 | 4.6 |
| | | 3910 | kl-10 | A | 0 | 0 | 0.2 | 0.4 | 34.6 | 0.4 | 10.6 | 0 | 3.3 | 4.8 |
| | | | | B | 0 | 0.9 | 0.4 | 0.5 | 55.4 | 0.3 | 2 | 0 | 4.1 | 6 |
| | | | | C | 0.1 | 0.3 | 0.7 | 0.7 | 17.4 | 0 | 0.1 | 0.1 | 0.9 | 5.9 |
| | | Transferable in 1/3 systems: | | | | | | | | | | | | |
| | | 4022 | kl-11 | A | 0.1 | 1.5 | 0.8 | 0.4 | 4.8 | 24.7 | 2 | 1.9 | 0.6 | 4.5 |
| | | | | B | 1.2 | 0.6 | 4.1 | 1.1 | 0.4 | 31.3 | 4.4 | 17.1 | 1.6 | 10.3 |
| | | | | C | 1.7 | 2.7 | 6 | 0.9 | 0.7 | 70.9 | 0.3 | 8.3 | 0 | 0.1 |

*System A: CAT-2200 H1/L1 & Pertuzumab H2/L2 (with 10:20:24:46 ratio H1:H2:L1:L2 DNA); System B: CAT-2200 H1/L1 & SGN-CD19a H2/L2 (with 15:15:35:35 ratio H1:H2:L1:L2 DNA); System C: CR8071 H1/L1 & SGN-CD19a H2/L2 with 15:15:35:35 ratio H1:H2:L1:L2 DNA)
**Considering full Ab species only
^{§}Designs are shaded with increasing grey according to their transferability in 3/3 (not shaded), 2/3, or 1/3 systems, based on a positive Change in % H1L1 and % H2L2 Pairing with respect to wild type
^{‡}Estimated change with respect to wild type
^{†}Design 3980 was assessed only in 2 systems

TABLE 15
LC-MS pairing data and post pA yields (mg/L) for K-L designs (total bispecific)

| | | | | | Change | | Change | | Change | | | | | |
|---|---|---|---|---|---|---|---|---|---|---|---|---|---|---|
| | | | | | in % | | in | | in % | | Change in | | | |
| | | | | | H1L1 | | % H2L2 | | H1L1 and | | H1L1_H2L2 | | | |
| | | | | | Pairing | | Pairing | % | % H2L2 | | and | | | |
| | | | | | with | | with | H1L1 | Pairing | H1L1_H2L2 | H1L2_H2L1** | H1L1_H2L2 | | |
| | | | | % | respect | % | respect | and % | with | and | with | and | | |
| | | | Post pA yield | H1L1 | to wild | H2L2 | to wild | H2L2 | respect to | H1L2_H2L1** | respect to | H1L2_H2L1 | H1L1_H1L1 | H1L1_H1L2 |
| Design^{§} | Cluster | System* | (mg/LA 280) | Pairing | type | Pairing | type | Pairing | wild type | [symbol] | wild type | [symbol] | [symbol] | [symbol] |

Transferable in 3/3 systems:

| | | | | | | | | | | | | | | |
|---|---|---|---|---|---|---|---|---|---|---|---|---|---|---|
| 2901 | kl-6 | A | 20 | 94.3 | 75.2 | 97.9 | 4.8 | 96.6 | 35.6 | 87 | 70.2 | 64.1 | 0.2 | 0.3 |
| | | B | 18 | 85.7 | 15.6 | 94.6 | 40.4 | 88.8 | 22.1 | 79.2 | 44.1 | 48.2 | 1.6 | 0.3 |
| | | C | 19 | 62.6 | 14.2 | 99.5 | 19.9 | 78.7 | 21.2 | 60.6 | 13.8 | 46.6 | 1.4 | 1.2 |
| 2979 | kl-4 | A | 20 | 99.4 | 47.6^{‡} | 98.3 | 27.2^{‡} | 98.8 | 31.5^{‡} | 95.7 | 63.8^{‡} | 83.8 | 1.5 | 0 |
| | | B | 30 | 98 | 6 | 77.3 | 39.7 | 86.7 | 15.5 | 72.4 | 40.3 | 63.7 | 0.2 | 0.1 |
| | | C | 14 | 94.5 | 12.2 | 99.5 | 39.6 | 98.5 | 26.7 | 79.9 | 31.8 | 34.3 | 0 | 0.7 |
| 3008 | kl-3 | A | 27 | 98.8 | 79.7 | 98.3 | 5.2 | 98.5 | 37.5 | 96.7 | 79.8 | 75.9 | 0 | 0.1 |
| | | B | 26 | 98.3 | 28.2 | 98.8 | 44.6 | 98.4 | 31.7 | 93.2 | 58.1 | 57 | 2.1 | 0 |
| | | C | 17 | 84.7 | 36.3 | 99.6 | 20 | 90.1 | 32.6 | 78.2 | 31.4 | 56.5 | 2.9 | 3.8 |
| 3018 | kl-3 | A | 23 | 97.1 | 78 | 98.7 | 5.6 | 98 | 37 | 95.3 | 78.4 | 72.4 | 0 | 0 |
| | | B | 33 | 96 | 25.9 | 98.6 | 44.4 | 96.5 | 29.8 | 77.9 | 42.8 | 33.9 | 7.1 | 0.1 |
| | | C | 18 | 79.7 | 11.6 | 99.5 | 28.2 | 84.3 | 15.4 | 70 | 18.1 | 34.3 | 3.4 | 0.8 |
| 3102 | kl-12 | A | 28 | 96.5 | 44.7^{‡} | 97.6 | 26.5^{‡} | 97.1 | 29.8^{‡} | 93.1 | 61.2^{‡} | 81.3 | 0.1 | 0 |
| | | B | 33 | 96.6 | 4.6 | 92.8 | 55.2 | 94.4 | 23.2 | 86.9 | 54.8 | 74.7 | 0.2 | 0.1 |
| | | C | 27 | 88.7 | 6.4 | 99.8 | 39.9 | 98.1 | 26.3 | 65.2 | 17.1 | 27.1 | 0.1 | 0.4 |
| 3109 | kl-8 | A | 16 | 99.7 | 47.9^{‡} | 97.4 | 26.3^{‡} | 98.6 | 31.3^{‡} | 96.4 | 64.5^{‡} | 81.3 | 0.8 | 0.1 |
| | | B | 35 | 99.1 | 29^{‡} | 97.1 | 42.9^{‡} | 98 | 31.3^{‡} | 94.1 | 59^{‡} | 78.4 | 0.3 | 0.1 |
| | | C | 21 | 84 | 15.9^{‡} | 99.6 | 28.3^{‡} | 97.9 | 29^{‡} | 66.8 | 18.7^{‡} | 17.6 | 0.1 | 0.3 |
| 3113 | kl-12 | A | 33 | 97 | 45.2^{‡} | 98.4 | 27.3^{‡} | 97.8 | 30.5^{‡} | 93.8 | 61.9^{‡} | 74.7 | 0.2 | 0 |
| | | B | 43 | 98.9 | 6.9 | 90.9 | 53.3 | 94.3 | 23.1 | 85.6 | 53.5 | 72.8 | 0.3 | 0.2 |
| | | C | 28 | 76.7 | −5.6 | 99.6 | 39.7 | 95.2 | 23.4 | 64 | 15.9 | 28.3 | 0 | 0.6 |
| 3890 | kl-9 | A | 29 | 99.8 | 48^{‡} | 87.5 | 16.4^{‡} | 93.3 | 26^{‡} | 85.1 | 53.2^{‡} | 70.7 | 1.7 | 0 |
| | | B | 24 | 99.7 | 29.6^{‡} | 51.3 | −2.9^{‡} | 73.7 | 7^{‡} | 47.7 | 12.6^{‡} | 41.4 | 1.1 | 0 |
| | | C | 27 | 98.4 | 30.3 | 88.5 | 17.2 | 92.8 | 23.9 | 86.3 | 34.4 | 72.3 | 0.7 | 0.3 |
| 3898 | kl-8 | A | 15 | 98.7 | 46.9^{‡} | 98.7 | 27.6^{‡} | 98.7 | 31.4^{‡} | 93.9 | 62^{‡} | 81.4 | 2.3 | 0 |
| | | B | 21 | 97.3 | 5.3 | 74.5 | 36.9 | 85.9 | 14.7 | 71.1 | 39 | 68.7 | 0.5 | 0.3 |
| | | C | 11 | 97.4 | 15.1 | 95.2 | 35.3 | 96.3 | 24.5 | 91.4 | 43.3 | 87.4 | 0.4 | 0.8 |
| 3910 | kl-10 | A | 20 | 99.6 | 47.8^{‡} | 57 | −14.1^{‡} | 79.1 | 11.8^{‡} | 54.1 | 22.2^{‡} | 43.9 | 1.8 | 0 |
| | | B | 41 | 99.6 | 7.6 | 39.7 | 2.1 | 66.9 | −4.3 | 34 | 1.9 | 29.9 | 0.5 | 0.1 |
| | | C | 52 | 99.3 | 17 | 81 | 21.1 | 89.5 | 17.7 | 78.7 | 30.6 | 73.2 | 0.3 | 0.2 |
| 3947 | kl-4 | A | 16 | 99.6 | 47.8^{‡} | 92.8 | 21.7^{‡} | 96.3 | 29^{‡} | 89.1 | 57.2^{‡} | 73.2 | 2.3 | 0 |
| | | B | 24 | 98.8 | 28.7^{‡} | 70.3 | 16.1^{‡} | 81.7 | 15^{‡} | 66.1 | 31^{‡} | 53.4 | 0.2 | 0.3 |
| | | C | 11 | 91.3 | 23.2 | 94.5 | 23.2 | 94 | 25.1 | 65.7 | 13.8 | 23.4 | 0.2 | 0.2 |
| 3951 | kl-4 | A | 14 | 98.7 | 46.9^{‡} | 89.5 | 18.4^{‡} | 93.2 | 25.9^{‡} | 83.5 | 51.6^{‡} | 63.3 | 0.2 | 0 |
| | | B | 23 | 99.8 | 29.7^{‡} | 77.5 | 23.3^{‡} | 85.6 | 18.9^{‡} | 72.8 | 37.7^{‡} | 52.5 | 0.1 | 0 |
| | | C | 23 | 97.5 | 29.4 | 97.1 | 25.8 | 97.3 | 28.4 | 92.2 | 40.3 | 73.6 | 0 | 0.2 |
| 3954 | kl-7 | A | 35 | 99.1 | 47.3^{‡} | 97.7 | 26.6^{‡} | 98.3 | 31^{‡} | 92.7 | 60.8^{‡} | 71.9 | 1 | 0 |
| | | B | 38 | 98.4 | 28.3^{‡} | 61.8 | 7.6^{‡} | 88.6 | 21.9^{‡} | 46 | 10.9^{‡} | 27.3 | 12.9 | 0.1 |
| | | C | 39 | 89 | 40.6 | 98.7 | 19.1 | 92.4 | 34.9 | 78.1 | 31.3 | 55.4 | 8.9 | 1.1 |
| 3957 | kl-7 | A | 32 | 90.2 | 71.1 | 90.6 | −2.5 | 90.4 | 29.5 | 78.8 | 61.9 | 66.8 | 0.6 | 0.1 |
| | | B | 34 | 99 | 28.9 | 85.4 | 31.2 | 95.6 | 28.9 | 70.9 | 35.8 | 36.5 | 7.9 | 0.1 |
| | | C | 41 | 87.2 | 38.8 | 99.7 | 20.1 | 90.8 | 33.3 | 75.5 | 28.7 | 45.7 | 8 | 0.9 |
| 3967 | kl-9 | A | 12 | 100 | 42.6 | 99.9 | 20 | 99.9 | 29.3 | 97.2 | 50.2 | 68.3 | 1.8 | 0 |
| | | B | 18 | 97.1 | 27^{‡} | 98.6 | 44.4^{‡} | 98 | 31.3^{‡} | 91.8 | 56.7^{‡} | 69.4 | 0.3 | 0.1 |
| | | C | 14 | 96.7 | 28.6 | 100 | 28.7 | 98.6 | 29.7 | 95.2 | 43.3 | 80 | 0.8 | 1.3 |
| 3971 | kl-2 | A | 27 | 98.9 | 79.8 | 99.1 | 6 | 99.1 | 38.1 | 87.4 | 70.6 | 45.6 | 0.2 | 0 |
| | | B | 27 | 97.9 | 27.8 | 99.3 | 45.1 | 98.9 | 32.2 | 93.2 | 58.1 | 62.3 | 0 | 0 |
| | | C | 30 | 89 | 40.6 | 99.8 | 20.2 | 92.5 | 35 | 82.6 | 35.8 | 54.2 | 3.9 | 0.7 |
| 3972 | kl-2 | A | 25 | 99.9 | 48.1^{‡} | 99.5 | 28.4^{‡} | 99.7 | 32.4^{‡} | 96.9 | 65^{‡} | 79.4 | 1.2 | 0 |
| | | B | 36 | 98.1 | 28^{‡} | 99.7 | 45.5^{‡} | 98.5 | 31.8^{‡} | 84 | 48.9^{‡} | 46.5 | 8.1 | 0.1 |
| | | C | 38 | 92.8 | 44.4 | 99.8 | 20.2 | 95.2 | 37.7 | 81.3 | 34.5 | 57.4 | 5.7 | 1.1 |
| 3974 | kl-l | A | 22 | 91.8 | 72.7 | 94.7 | 1.6 | 93.5 | 32.5 | 83.7 | 66.8 | 62.3 | 0.4 | 4.4 |
| | | B | 38 | 97.6 | 27.5 | 92.6 | 38.4 | 96.5 | 29.8 | 72.9 | 37.8 | 34 | 8.3 | 0.3 |
| | | C | 36 | 59.9 | 11.5 | 99.8 | 20.2 | 71 | 13.5 | 49.5 | 2.7 | 31.4 | 5.6 | 4.5 |
| 3980^{†} | kl-2 | A | 26 | 99.7 | 47.9^{‡} | 74.8 | 3.7^{‡} | 86.9 | 19.6^{‡} | 71.4 | 39.5^{‡} | 59 | 1.9 | 0.1 |
| | | B | 37 | 99.8 | 29.7^{‡} | 82.6 | 28.4^{‡} | 95.1 | 28.4^{‡} | 69.6 | 34.5^{‡} | 39.6 | 8.3 | 0 |
| 3987 | kl-3 | A | 25 | 99.7 | 47.9^{‡} | 83 | 11.9^{‡} | 93.9 | 26.6^{‡} | 80.7 | 48.8^{‡} | 51.6 | 2.7 | 0 |
| | | B | 27 | 97.5 | 27.4^{‡} | 98.1 | 43.9^{‡} | 97.6 | 30.9^{‡} | 66.4 | 31.3^{‡} | 24.8 | 11.1 | 0.3 |
| | | C | 21 | 99.2 | 50.8 | 99.3 | 19.7 | 99.2 | 41.7 | 78.9 | 32.1 | 37.4 | 9.2 | 0.4 |
| 4008 | kl-8 | A | 22 | 100 | 42.6 | 99.3 | 19.4 | 99.7 | 29.1 | 96.5 | 49.5 | 81.6 | 1.2 | 0 |
| | | B | 30 | 99.6 | 7.6 | 70.9 | 33.3 | 84.6 | 13.4 | 68.7 | 36.6 | 60.2 | 0.6 | 0 |
| | | C | 26 | 99.6 | 17.3 | 98.3 | 38.4 | 98.9 | 27.1 | 96.8 | 48.7 | 85.5 | 0.1 | 0.3 |
| 4010 | kl-12 | A | 29 | 99.9 | 42.5 | 98.9 | 19 | 99.5 | 28.9 | 98.1 | 51.1 | 76.7 | 0.2 | 0 |
| | | B | 27 | 99.5 | 7.5 | 71.5 | 33.9 | 84.8 | 13.6 | 67.1 | 35 | 59.9 | 0.5 | 0.1 |
| | | C | 23 | 99.3 | 17 | 98.6 | 38.7 | 98.9 | 27.1 | 96.7 | 48.6 | 83.6 | 0.2 | 0.4 |
| 4040 | kl-8 | A | 29 | 99.8 | 48^{‡} | 97.7 | 26.6^{‡} | 98.7 | 31.4^{‡} | 94.6 | 62.7^{‡} | 85.8 | 1.2 | 0 |
| | | B | 41 | 99.2 | 29.1^{‡} | 75.8 | 21.6^{‡} | 86 | 19.3^{‡} | 73.7 | 38.6^{‡} | 62.7 | 0.6 | 0 |
| | | C | 19 | 98.7 | 30.6 | 98.6 | 27.3 | 98.7 | 29.8 | 81.8 | 29.9 | 51.2 | 1.2 | 0.1 |
| 4320 | kl-10 | A | 14 | 99.7 | 47.9^{‡} | 91.4 | 20.3^{‡} | 95.4 | 28.1^{‡} | 89.1 | 57.2^{‡} | 77.7 | 0.6 | 0 |
| | | B | 35 | 99.8 | 7.8 | 55.5 | 17.9 | 73.4 | 2.2 | 47.9 | 15.8 | 38.9 | 0.3 | 0.1 |
| | | C | 22 | 96.6 | 14.3 | 81.4 | 21.5 | 89 | 17.2 | 77.1 | 29 | 75 | 0.6 | 3.2 |

Transferable in 2/3 systems:

| | | | | | | | | | | | | | | |
|---|---|---|---|---|---|---|---|---|---|---|---|---|---|---|
| 2892 | kl-6 | A | 28 | 81.3 | 62.2 | 98.3 | 5.2 | 94.1 | 33.1 | 71.8 | 54.9 | 39.1 | 0.1 | 0.2 |
| | | B | 34 | 84.7 | 14.6 | 94.3 | 40.1 | 88.7 | 22 | 84.2 | 49.1 | 63.4 | 0.6 | 0.1 |
| | | C | 32 | 41.5 | −6.9 | 99.6 | 20 | 69.9 | 12.4 | 41 | −5.8 | 34.3 | 0.1 | 0.6 |
| 2994 | kl-3 | A | 35 | 78.3 | 59.2 | 98.9 | 5.8 | 92 | 31 | 70.1 | 53.2 | 50 | 0.7 | 0 |
| | | B | 42 | 84.8 | 14.7 | 99.2 | 45 | 90.9 | 24.2 | 80.9 | 45.8 | 64.6 | 3.5 | 0.3 |
| | | C | 33 | 26.3 | −22.1 | 99.7 | 20.1 | 57.4 | −0.1 | 25.5 | −21.3 | 19.9 | 0.3 | 1.6 |
| 3025 | kl-2 | A | 35 | 83.6 | 64.5 | 99.1 | 6 | 93.9 | 33 | 72.7 | 55.9 | 54.9 | 0.1 | 0 |
| | | B | 30 | 80.2 | 10.1 | 87.9 | 33.7 | 85.5 | 18.8 | 71.4 | 36.3 | 44.9 | 0 | 0 |
| | | C | 37 | 43.9 | −4.5 | 99.7 | 20.1 | 70.1 | 12.6 | 41.1 | −5.7 | 40.4 | 1.8 | 1.4 |
| 3122 | kl-5 | A | 16 | 85.9 | 34.1^{‡} | 23.4 | −47.7^{‡} | 85.8 | 18.5^{‡} | 0 | −31.9^{‡} | 0 | 10.4 | 2.5 |
| | | B | 38 | 96.9 | 4.9 | 89.7 | 52.1 | 92.9 | 21.7 | 84.7 | 52.6 | 72.7 | 0.2 | 0.2 |
| | | C | 45 | 65.8 | −16.5 | 99.8 | 39.9 | 91.1 | 19.3 | 61.5 | 13.4 | 32.6 | 0.1 | 0.4 |
| 3878 | kl-8 | A | 21 | 99.7 | 47.9^{‡} | 89.7 | 18.6^{‡} | 94.6 | 27.3^{‡} | 87.6 | 55.7^{‡} | 78.1 | 0.6 | 0 |
| | | B | 31 | 97.7 | 5.7 | 28.7 | −8.9 | 71.8 | 0.6 | 24.2 | −7.9 | 17.3 | 3.6 | 0.7 |
| | | C | 30 | 99.3 | 17 | 67.4 | 7.5 | 88.2 | 16.4 | 61.3 | 13.2 | 42.6 | 6 | 0.6 |
| 3931 | kl-7 | A | 30 | 92.2 | 73.1 | 94.9 | 1.8 | 93.6 | 32.6 | 83.9 | 67.1 | 70.7 | 0.5 | 0.2 |
| | | B | 39 | 85.5 | 15.4 | 83.6 | 29.4 | 85 | 18.3 | 60.7 | 25.6 | 35.4 | 6.8 | 1.7 |
| | | C | 40 | 57.1 | 8.7 | 99.5 | 19.9 | 68.6 | 11.1 | 45.7 | −1.1 | 27.3 | 4.6 | 3.5 |
| 3997 | kl-1 | A | 14 | 99.8 | 48^{‡} | 90.7 | 19.6^{‡} | 94.9 | 27.6^{‡} | 85.9 | 54^{‡} | 69 | 0.6 | 0 |
| | | B | 25 | 98.8 | 28.7^{‡} | 53 | −1.2^{‡} | 90.6 | 23.9^{‡} | 38 | 2.9^{‡} | 16 | 12.2 | 0.2 |
| | | C | 6 | 96 | 27.9 | 99.9 | 28.6 | 96 | 27.1 | 2.9 | −49 | 0.4 | 11.2 | 0.7 |
| 4022 | kl-11 | A | 10 | 70.4 | 18.6^{‡} | 90.4 | 19.3^{‡} | 80.6 | 13.3^{‡} | 62.9 | 31^{‡} | 57.3 | 1.3 | 0.2 |
| | | B | 20 | 35.4 | −34.7^{‡} | 90.4 | 36.2^{‡} | 61.3 | −5.4^{‡} | 40.6 | 5.5^{‡} | 27 | 0.4 | 0.5 |
| | | C | 21 | 10 | −38.4 | 87.6 | 8 | 48 | −9.5 | 7.7 | −39.1 | 7 | 0.5 | 1 |

Transferable in 1/3 systems:

| 3041 | kl-3 | A | 8 | 66.7 | 47.6 | 23.5 | −69.6 | 66.6 | 5.6 | 0 | −16.9 | 0 | 5.4 | 2.1 |
|---|---|---|---|---|---|---|---|---|---|---|---|---|---|---|
| | | B | 29 | 75.6 | 5.5 | 99.6 | 45.4 | 92.2 | 25.5 | 71 | 35.9 | 46.3 | 0.1 | 0 |
| | | C | 26 | 27.2 | −21.2 | 99.9 | 20.3 | 54.4 | −3.1 | 29 | −17.8 | 20.9 | 0.8 | 1.9 |
| | | | | | H1L2_H1L2 | H2L1_H2L1 | H2L1_H2L2 | H2L2_H2L2 | H1L1_H2L1 | H1L2_H2L2 | H1L1 | H1L2 | H2L1 | H2L2 |
| | | Design^{§} | Cluster | System* | [symbol] | [symbol] | [symbol] | [symbol] | [symbol] | [symbol] | [symbol] | [symbol] | [symbol] | [symbol] |
| | | Transferable in 3/3 systems: | | | | | | | | | | | | |
| | | 2901 | kl-6 | A | 0.1 | 0.3 | 1.5 | 3.5 | 0.4 | 3.4 | 2.3 | 0.2 | 0.1 | 23.7 |
| | | | | B | 0.3 | 0.3 | 0.2 | 0.2 | 2.2 | 7.7 | 29 | 5 | 0.4 | 4.7 |
| | | | | C | 1.4 | 0.1 | 0 | 0.5 | 0 | 25.7 | 10 | 6.3 | 0.1 | 6.7 |
| | | 2979 | kl-4 | A | 0.2 | 0.9 | 0 | 1 | 0 | 0.2 | 5.4 | 0 | 0 | 7.1 |
| | | | | B | 0.8 | 0.1 | 0 | 0.8 | 22.1 | 0 | 1.2 | 0 | 1.2 | 9.6 |
| | | | | C | 0 | 0.1 | 0 | 6 | 0.5 | 1.3 | 0 | 0 | 0.1 | 56.9 |
| | | 3008 | kl-3 | A | 0.2 | 0.1 | 0.3 | 0.2 | 1.3 | 0.5 | 2.1 | 0.1 | 0.1 | 19.3 |
| | | | | B | 0.2 | 0.1 | 0 | 0.1 | 0.2 | 1.4 | 35.8 | 0.3 | 0.1 | 2.7 |
| | | | | C | 0.5 | 0 | 0.1 | 0.3 | 0.1 | 8.1 | 20.8 | 3.4 | 0 | 3.5 |
| | | 3018 | kl-3 | A | 0.1 | 0.1 | 0 | 1 | 0.6 | 1.7 | 3.1 | 0.2 | 0.4 | 20.3 |
| | | | | B | 0.2 | 0.1 | 0 | 0 | 0.2 | 18 | 52.9 | 2.1 | 0.1 | 1.4 |
| | | | | C | 0.3 | 0 | 0 | 0 | 0.2 | 10.1 | 40.4 | 9.8 | 0 | 0.8 |
| | | 3102 | kl-12 | A | 0.5 | 1.2 | 0.2 | 18 | 0 | 2.1 | 2.8 | 0 | 0 | 9.9 |
| | | | | B | 1.3 | 0.1 | 0 | 2 | 7.4 | 0 | 0.1 | 0 | 0.3 | 13.7 |
| | | | | C | 0 | 0.1 | 0 | 11 | 0.1 | 2.9 | 0 | 0.1 | 0 | 58.2 |
| | | 3109 | kl-8 | A | 0 | 0.5 | 1.2 | 0.3 | 0 | 0.2 | 10.7 | 0 | 0.1 | 4.8 |
| | | | | B | 0 | 0 | 0 | 1.1 | 2.8 | 0.6 | 0.2 | 0 | 0.2 | 16.2 |
| | | | | C | 0.1 | 0.1 | 0 | 5.6 | 0 | 2.6 | 0.1 | 0.2 | 0.2 | 73.1 |
| | | 3113 | kl-12 | A | 0 | 0.9 | 0 | 1.3 | 0.1 | 2.4 | 2.1 | 0 | 0 | 18.2 |
| | | | | B | 0.1 | 0 | 0 | 1.7 | 9.5 | 0.4 | 1.1 | 0 | 0.4 | 13.4 |
| | | | | C | 0.1 | 0.2 | 0 | 7.8 | 0.3 | 7 | 0.1 | 0.6 | 0 | 55 |
| | | 3890 | kl-9 | A | 0 | 0 | 0 | 1.2 | 9.2 | 0.1 | 5.4 | 0 | 1.9 | 9.5 |
| | | | | B | 0 | 0.2 | 0.1 | 0.4 | 43.4 | 0.1 | 2.7 | 0 | 4.2 | 6.4 |
| | | | | C | 0.3 | 0 | 0.2 | 0.9 | 8.5 | 0.5 | 2.1 | 0 | 2 | 12.1 |
| | | 3898 | kl-8 | A | 0 | 0.6 | 0 | 1 | 0 | 1.3 | 9.1 | 0 | 0 | 4.3 |
| | | | | B | 1 | 0 | 0 | 0.8 | 25 | 0.2 | 1.1 | 0 | 0.2 | 2.1 |
| | | | | C | 0 | 0 | 0.1 | 0.6 | 4.8 | 1.6 | 1 | 0.1 | 0 | 3.2 |
| | | 3910 | kl-10 | A | 0 | 0 | 0.2 | 0.4 | 34.6 | 0.4 | 10.6 | 0 | 3.3 | 4.8 |
| | | | | B | 0 | 0.9 | 0.4 | 0.5 | 55.4 | 0.3 | 2 | 0 | 4.1 | 6 |
| | | | | C | 0.1 | 0.3 | 0.7 | 0.7 | 17.4 | 0 | 0.1 | 0.1 | 0.9 | 5.9 |
| | | 3947 | kl-4 | A | 0 | 0.1 | 0.1 | 1.2 | 5 | 0.4 | 9.5 | 0 | 0.9 | 7.4 |
| | | | | B | 0 | 0.3 | 0.5 | 1.9 | 23.6 | 0.6 | 0.7 | 0 | 5.5 | 13 |
| | | | | C | 0.1 | 0 | 0.1 | 8 | 1.7 | 2 | 2.4 | 0.3 | 3.7 | 57.9 |
| | | 3951 | kl-4 | A | 0.3 | 1.7 | 0 | 3 | 6.8 | 0.3 | 4.2 | 0 | 1.1 | 18.8 |
| | | | | B | 0 | 0.1 | 0.2 | 1.5 | 17.7 | 0.1 | 0.9 | 0 | 5.3 | 21.6 |
| | | | | C | 0 | 0 | 0 | 2.6 | 1.6 | 1.8 | 0.5 | 0 | 0.9 | 18.8 |
| | | 3954 | kl-7 | A | 0 | 0.5 | 0 | 2.1 | 1.2 | 0.8 | 4.2 | 0 | 0.2 | 18 |
| | | | | B | 0.1 | 0 | 0 | 0.1 | 17.6 | 1.1 | 36.6 | 0.4 | 1.4 | 2.2 |
| | | | | C | 0.1 | 0 | 0 | 0.2 | 0.6 | 4.6 | 20.5 | 4.2 | 0.1 | 4.3 |
| | | 3957 | kl-7 | A | 0 | 0.1 | 0.1 | 0.4 | 7.9 | 8.6 | 8.9 | 0.7 | 0.4 | 5.3 |
| | | | | B | 0.1 | 0.1 | 0 | 0 | 6.4 | 0.3 | 44.4 | 0.4 | 0.4 | 3.3 |
| | | | | C | 0.2 | 0 | 0 | 0.2 | 0 | 5.5 | 31.2 | 5.8 | 0.1 | 2.4 |
| | | 3967 | kl-9 | A | 0 | 0 | 0 | 0 | 0 | 0 | 21.8 | 0 | 0 | 7.9 |
| | | | | B | 0 | 0 | 0.1 | 2 | 1.7 | 2.1 | 1.1 | 0 | 0 | 23.3 |
| | | | | C | 0 | 0 | 0 | 0.7 | 0 | 1.3 | 0.6 | 0.2 | 0 | 15.3 |
| | | 3971 | kl-2 | A | 0 | 0.1 | 0.3 | 5.1 | 0.7 | 0.1 | 0.2 | 0.2 | 0.1 | 17.4 |
| | | | | B | 0.2 | 0.1 | 0 | 3 | 0.3 | 1 | 0.7 | 0 | 0.2 | 32.2 |
| | | | | C | 0.2 | 0 | 0 | 0.1 | 0.1 | 6.5 | 28.4 | 3.6 | 0 | 2.3 |
| | | 3972 | kl-2 | A | 0 | 0.1 | 0 | 0.9 | 0.2 | 0.1 | 12.1 | 0 | 0 | 6 |
| | | | | B | 0.1 | 0 | 0 | 0.3 | 0.1 | 0.2 | 40 | 1.1 | 0 | 3.5 |
| | | | | C | 0 | 0 | 0 | 0.2 | 0.1 | 6 | 25.2 | 1.1 | 0 | 3.1 |
| | | 3974 | kl-l | A | 0 | 0.3 | 0.4 | 1.9 | 2.8 | 2 | 4.1 | 0.3 | 1.1 | 20 |
| | | | | B | 0.2 | 0.2 | 0 | 0 | 2.7 | 1 | 49.3 | 1.1 | 0.1 | 3 |
| | | | | C | 18 | 0 | 0 | 0.1 | 0 | 20.1 | 19.7 | 14.9 | 0 | 1.9 |
| | | 3980^{†} | kl-2 | A | 0 | 1.1 | 0.1 | 1.4 | 18.9 | 0.1 | 7.8 | 0.1 | 2.3 | 7.2 |
| | | | | B | 0 | 0.1 | 0.1 | 0.2 | 8.6 | 0.1 | 40.2 | 0.1 | 0.3 | 2.4 |
| | | 3987 | kl-3 | A | 0.1 | 1.2 | 0.4 | 0.1 | 7.7 | 0.1 | 33 | 0.1 | 0.6 | 2.3 |
| | | | | B | 0.2 | 0 | 0 | 0.1 | 0.4 | 0.4 | 60.4 | 1.6 | 0 | 0.7 |
| | | | | C | 0 | 0 | 0 | 0.1 | 0 | 0.2 | 51.8 | 0.3 | 0.1 | 0.4 |
| | | 4008 | kl-8 | A | 0 | 0 | 0 | 1 | 0.6 | 0 | 8.7 | 0 | 0 | 6.9 |
| | | | | B | 0.1 | 0.1 | 0.1 | 0.7 | 25.7 | 0.1 | 4.1 | 0 | 2.2 | 6.1 |
| | | | | C | 0 | 0 | 0 | 0.7 | 1.7 | 0 | 1.3 | 0 | 0.1 | 10.3 |
| | | 4010 | kl-12 | A | 0 | 0.1 | 0.1 | 0.4 | 0.7 | 0 | 15.3 | 0 | 0 | 6.6 |
| | | | | B | 0.1 | 0.1 | 0.3 | 0.4 | 27.7 | 0.1 | 3 | 0 | 0.9 | 6.9 |
| | | | | C | 0 | 0 | 0 | 0.8 | 1.4 | 0 | 0.8 | 0 | 0.1 | 12.6 |
| | | 4040 | kl-8 | A | 0 | 0.9 | 0 | 2 | 0.7 | 0.1 | 3.7 | 0 | 0 | 5.5 |
| | | | | B | 0 | 0.1 | 0.1 | 0.6 | 20.4 | 0.6 | 1.3 | 0 | 3.3 | 10.3 |
| | | | | C | 0 | 0 | 0.3 | 8.2 | 1 | 0.5 | 0.5 | 0 | 0.3 | 36.7 |
| | | 4320 | kl-10 | A | 0 | 0.1 | 0.1 | 1.3 | 7.3 | 0.2 | 5 | 0 | 0.7 | 7.1 |
| | | | | B | 0 | 1 | 0.3 | 0.7 | 39.8 | 0.1 | 0.8 | 0 | 5.4 | 12.6 |
| | | | | C | 0 | 0.1 | 0.7 | 0.9 | 16.8 | 0.1 | 0.2 | 0.1 | 0.5 | 2 |
| | | Transferable in 2/3 systems: | | | | | | | | | | | | |
| | | 2892 | kl-6 | A | 0.2 | 0.3 | 1 | 5 | 0.2 | 8.3 | 0.4 | 0.2 | 0.3 | 14.6 |
| | | | | B | 0.5 | 0.3 | 0.2 | 0.1 | 3.2 | 7 | 15.8 | 4.9 | 0.3 | 3.7 |
| | | | | C | 0.8 | 0 | 0 | 0.1 | 0.2 | 47.5 | 3.6 | 5.1 | 0 | 7.6 |
| | | 2994 | kl-3 | A | 0 | 0.2 | 1 | 5.1 | 0.1 | 14.2 | 0.6 | 0.1 | 0 | 28 |
| | | | | B | 0.3 | 0.2 | 0 | 0.1 | 0.2 | 10.6 | 12.9 | 3 | 0 | 4.2 |
| | | | | C | 3.1 | 0 | 0 | 0.1 | 0 | 53.1 | 4.1 | 12 | 0.1 | 5.7 |
| | | 3025 | kl-2 | A | 0 | 0.1 | 0.4 | 8.9 | 0.4 | 10.7 | 0.1 | 0.1 | 0.1 | 24.1 |
| | | | | B | 0.1 | 0.1 | 0.1 | 2 | 3.6 | 12 | 0.6 | 0 | 6.4 | 30.1 |
| | | | | C | 2.3 | 0.1 | 0 | 0.2 | 0.1 | 52.1 | 0.6 | 0.7 | 0 | 0.3 |
| | | 3122 | kl-5 | A | 0.5 | 0 | 0 | 0 | 0.2 | 0 | 74 | 12.3 | 0 | 0 |
| | | | | B | 0.5 | 0.1 | 0 | 0.8 | 10.2 | 1 | 1.6 | 0.3 | 0.5 | 11.8 |
| | | | | C | 0.5 | 0.1 | 0 | 3.4 | 0.2 | 15.9 | 0.2 | 0.1 | 0 | 16.7 |
| | | 3878 | kl-8 | A | 0 | 0.2 | 0.3 | 1 | 8.7 | 0.2 | 4.7 | 0 | 0.6 | 5.5 |
| | | | | B | 1 | 0.9 | 0.1 | 0.2 | 47.8 | 0.1 | 24.5 | 0 | 2 | 1.9 |
| | | | | C | 0.1 | 0 | 0.2 | 0.1 | 19.7 | 0.1 | 27.3 | 0 | 1.4 | 1.9 |
| | | 3931 | kl-7 | A | 0 | 0.2 | 1.7 | 1.5 | 3 | 6.5 | 7.2 | 0.4 | 0.1 | 8 |
| | | | | B | 0.2 | 0.2 | 0 | 0 | 7.9 | 6 | 32.6 | 6.4 | 0.3 | 2.3 |
| | | | | C | 2.2 | 0 | 0 | 0 | 0.1 | 22 | 21.6 | 16.3 | 0.1 | 2.2 |
| | | 3997 | kl-1 | A | 0.1 | 0.4 | 0 | 1.1 | 9.1 | 0.1 | 6.6 | 0 | 0 | 13 |
| | | | | B | 0.1 | 0 | 0 | 0 | 13.6 | 0 | 54 | 0.8 | 1.6 | 1.4 |
| | | | | C | 0.1 | 0 | 0 | 0 | 0 | 0.4 | 83.8 | 3.3 | 0 | 0.1 |
| | | 4022 | kl-11 | A | 0.1 | 1.5 | 0.8 | 0.4 | 4.8 | 24.7 | 2 | 1.9 | 0.6 | 4.5 |
| | | | | B | 1.2 | 0.6 | 4.1 | 1.1 | 0.4 | 31.3 | 4.4 | 17.1 | 1.6 | 10.3 |
| | | | | C | 1.7 | 2.7 | 6 | 0.9 | 0.7 | 70.9 | 0.3 | 8.3 | 0 | 0.1 |
| | | Transferable in 1/3 systems: | | | | | | | | | | | | |
| | | 3041 | kl-3 | A | 1.4 | 0 | 0 | 0 | 0 | 0 | 60.2 | 30.7 | 0.2 | 0.1 |
| | | | | B | 0.3 | 0.2 | 0 | 3.8 | 0.1 | 14.5 | 0.1 | 0 | 0.1 | 34.6 |
| | | | | C | 4 | 0 | 0 | 0.1 | 0 | 44.3 | 4.8 | 18.5 | 0 | 4.6 |

*System A: CAT-2200 H1/L1 & Pertuzumab H2/L2 (with 10:20:24:46 ratio H1:H2:L1:L2 DNA); System B: CAT-2200 H1/L1 & SGN-CD19a H2/L2 (with 15:15:35:35 ratio H1:H2:L1:L2 DNA); System C: CR8071 H1/L1 & SGN-CD19a H2/L2 (with 15:15:35:35 ratio H1:H2:L1:L2 DNA)
**Considering full Ab species only
^{§}Designs are shaded according to their transferability in 3/3 (not shaded), or 2/3 (light grey) systems, based on a positive Change in H1L1_H2L2 and H1L2_H2L1** with respect to wild type
^{‡}Estimated change with respect to wild type
^{†}Design 1980 was assessed only in 2 systems

TABLE 16
LC-MS pairing data and post pA yields (mg/L) for K-K-derived K-L designs (total pairing)

| | | | | | | | | |
|---|---|---|---|---|---|---|---|---|
| | | | | | Change | | Change | |
| | | | | | in % | | in | |
| | | | | | H1L1 | | % H2L2 | |
| | | | Post | | Pairing | | Pairing | % |
| | | | pA | | with | | with | H1L1 |
| | | | yield | % | respect | % | respect | and % |
| | | | (mg/L | H1L1 | to wild | H2L2 | to wild | H2L2 |
| Design^{§} | Cluster | System* | A280) | Pairing | type | Pairing | type | Pairing |

Transferable in 3/3 systems:

| | | | | | | | | |
|---|---|---|---|---|---|---|---|---|
| 34 | kk-6 | A | 37 | 85.9 | 34.1^{‡} | 98.8 | 27.7^{‡} | 92.8 |
| | | B | 56 | 94.6 | 24.5^{‡} | 93.8 | 39.6^{‡} | 94.1 |
| | | C | 45 | 92.6 | 24.5 | 99.1 | 27.8 | 96.6 |
| 2826 | kk-2 | A | 38 | 23.1 | 4 | 99.6 | 6.5 | 67.8 |
| | | B | 44 | 53.7 | −16.4 | 89.7 | 35.5 | 75.3 |
| | | C | 28 | 27.8 | −40.3 | 98 | 26.7 | 69.6 |

Transferable in 2/3 systems:

| | | | | | | | | |
|---|---|---|---|---|---|---|---|---|
| 2188 | kk-10 | A | 19 | 28.5 | 9.4 | 98 | 4.9 | 69.8 |
| | | B | 21 | 31 | −39.1 | 92.9 | 38.7 | 54 |
| | | C | 16 | 24.9 | −23.5 | 99.5 | 19.9 | 66.2 |
| 2816 | kk-4 | A | 37 | 99.6 | 47.8^{‡} | 63.1 | −8^{‡} | 72.1 |
| | | B | 41 | 94.4 | 2.4 | 25.1 | -12.5 | 55.6 |
| | | C | 47 | 99.3 | 17 | 79.7 | 19.8 | 88.5 |
| 2827 | kk-3 | A | 37 | 97.9 | 46.1^{‡} | 93.6 | 22.5^{‡} | 95.5 |
| | | B | 43 | 98.9 | 6.9 | 22.3 | −15.3 | 56 |
| | | C | 41 | 98.2 | 15.9 | 85 | 25.1 | 88.5 |
| 2830 | kk-l | A | 24 | 76.8 | 25^{‡} | 99.6 | 28.5^{‡} | 90.9 |
| | | B | 23 | 83.8 | 13.7^{‡} | 98.5 | 44.3^{‡} | 88.7 |
| | | C | 25 | 28 | −20.4 | 99.6 | 20 | 46.5 |

Transferable in 1/3 systems:

| 2798 | kk-8 | A | 64 | 89.5 | 37.7^{‡} | 88 | 16.9^{‡} | 88.3 |
|---|---|---|---|---|---|---|---|---|
| | | B | 54 | 94 | 2 | 37.8 | 0.2 | 61.4 |
| | | C | 51 | 97.6 | 15.3 | 34.5 | −25.4 | 66.1 |
| | | | Change | | | | | |
| | | | in % | | Change in | | | |
| | | | H1L1 and | | H1L1_H2L2 | | | |
| | | | % H2L2 | | and | | | |
| | | | Pairing | H1L1_H2L2 | H1L2_H2L1** | H1L1_H2L2 | | |
| | | | with | and | with | and | | |
| | | | respect to | H1L2_H2L1** | respect to | H1L2_H2L1 | H1L1_H1L1 | H1L1_H1L2 |
| Design^{§} | Cluster | System* | wild type | [symbol] | wild type | [symbol] | [symbol] | [symbol] |

Transferable in 3/3 systems:

| | | | | | | | | |
|---|---|---|---|---|---|---|---|---|
| 34 | kk-6 | A | 25.5^{‡} | 82.3 | 50.4^{‡} | 71.9 | 0.6 | 0.2 |
| | | B | 27.4^{‡} | 90.1 | 55^{‡} | 73.3 | 0.1 | 0.1 |
| | | C | 27.7 | 86.4 | 34.5 | 66.1 | 1 | 1.1 |
| 2826 | kk-2 | A | 6.8 | 20.5 | 3.6 | 16.5 | 0.4 | 0.1 |
| | | B | 8.6 | 48.3 | 13.2 | 38.8 | 0 | 0.1 |
| | | C | 0.7 | 25.9 | −26 | 20.4 | 0.2 | 0.7 |

Transferable in 2/3 systems:

| | | | | | | | | |
|---|---|---|---|---|---|---|---|---|
| 2188 | kk-10 | A | 8.8 | 27.3 | 10.4 | 22 | 0 | 0.1 |
| | | B | −12.7 | 31.2 | −3.9 | 21.1 | 0.4 | 0.9 |
| | | C | 8.7 | 24.6 | −22.2 | 21.3 | 0.1 | 0.4 |
| 2816 | kk-4 | A | 4.8^{‡} | 58 | 26.1^{‡} | 32.2 | 0 | 0 |
| | | B | −15.6 | 23.1 | −9 | 19.5 | 0.4 | 1.4 |
| | | C | 16.7 | 78.5 | 30.4 | 69.4 | 0.2 | 0.3 |
| 2827 | kk-3 | A | 28.2^{‡} | 90.8 | 58.9^{‡} | 75 | 0.3 | 0 |
| | | B | −15.2 | 19.1 | −13 | 15.9 | 0.5 | 0.5 |
| | | C | 16.7 | 76.2 | 28.1 | 44.4 | 0.1 | 0.2 |
| 2830 | kk-l | A | 23.6^{‡} | 72.2 | 40.3^{‡} | 56.6 | 0.1 | 0.1 |
| | | B | 22^{‡} | 74.7 | 39.6^{‡} | 49.7 | 3.7 | 1.5 |
| | | C | −11 | 22 | −24.8 | 12.8 | 0.7 | 3.7 |

Transferable in 1/3 systems:

| 2798 | kk-8 | A | 21^{‡} | 61.6 | 29.7^{‡} | 36.1 | 0 | 0 |
|---|---|---|---|---|---|---|---|---|
| | | B | −9.8 | 33.8 | 1.7 | 28.1 | 0.2 | 0.9 |
| | | C | −5.7 | 32.4 | −15.7 | 30.6 | 1.2 | 0.1 |
| | | | H1L2_H1L2 | H2L1_H2L1 | H2L1_H2L2 | H2L2_H2L2 | H1L1_H2L1 | H1L2_H2L2 |
| Design^{§} | Cluster | System* | [symbol] | [symbol] | [symbol] | [symbol] | [symbol] | [symbol] |

Transferable in 3/3 systems:

| | | | | | | | | |
|---|---|---|---|---|---|---|---|---|
| 34 | kk-6 | A | 0 | 0.1 | 0.2 | 1.9 | 0.6 | 11.8 |
| | | B | 0 | 0.1 | 0.1 | 2 | 1.6 | 4.1 |
| | | C | 0 | 0 | 0.1 | 3.4 | 0.3 | 4.4 |
| 2826 | kk-2 | A | 0 | 0.1 | 0.1 | 2.4 | 0 | 61 |
| | | B | 0.1 | 0.4 | 0.2 | 1.3 | 3.6 | 35.9 |
| | | C | l | 0 | 0 | 1.3 | 0.2 | 55.1 |

Transferable in 2/3 systems:

| | | | | | | | | |
|---|---|---|---|---|---|---|---|---|
| 2188 | kk-10 | A | 0.1 | 0.2 | 0.6 | 3.5 | 0.3 | 53.6 |
| | | B | 1.8 | 0.1 | 0 | 0.1 | 3.1 | 40.3 |
| | | C | 1.5 | 0 | 0 | 1 | 0.1 | 62.2 |
| 2816 | kk-4 | A | 0.1 | 1 | 1.8 | 3.6 | 16.8 | 0 |
| | | B | 1.7 | 1.3 | 0.2 | 0.2 | 59.7 | 0.2 |
| | | C | 0 | 0.1 | 0.2 | 0.6 | 17.4 | 0.2 |
| 2827 | kk-3 | A | 0 | 0.8 | 0 | 2.2 | 4 | 0.4 |
| | | B | 0.1 | 0.3 | 0.1 | 0.4 | 65.3 | 0.2 |
| | | C | 0.1 | 0.1 | 0.8 | 5.2 | 7.3 | 0.2 |
| 2830 | kk-l | A | 0 | 0.2 | 0.1 | 4.2 | 0 | 17.1 |
| | | B | 1.4 | 0.1 | 0 | 0.2 | 0.7 | 9.2 |
| | | C | 5.7 | 0 | 0 | 0 | 0 | 35.2 |

Transferable in 1/3 systems:

| 2798 | kk-8 | A | 0 | 0.5 | 1.7 | 10.9 | 4.6 | 4.8 |
|---|---|---|---|---|---|---|---|---|
| | | B | 0.9 | 1.6 | 0.2 | 0.5 | 48.2 | 2.3 |
| | | C | 0.2 | 0.3 | 0.1 | 0.2 | 60 | 1.4 |
| | | | | | H1L1 | H1L2 | H2L1 | H2L2 |
| | | Design^{§} | Cluster | System* | [symbol] | [symbol] | [symbol] | [symbol] |
| | | Transferable in 3/3 systems: | | | | | | |
| | | 34 | kk-6 | A | 2.5 | 0.5 | 0.1 | 9.5 |
| | | | | B | 0.4 | 0.1 | 2.8 | 15.4 |
| | | | | C | 0 | 0 | 0.4 | 23 |
| | | 2826 | kk-2 | A | 0.9 | 1.4 | 0 | 17 |
| | | | | B | 0.2 | 0.4 | 4 | 15.1 |
| | | | | C | 0.4 | 0.4 | 1.1 | 19.3 |
| | | Transferable in 2/3 systems: | | | | | | |
| | | 2188 | kk-10 | A | 0.4 | 2.1 | 0.5 | 16.6 |
| | | | | B | 6.6 | 20.9 | 0.9 | 3.7 |
| | | | | C | 0.1 | 0.7 | 0.2 | 12.3 |
| | | 2816 | kk-4 | A | 0.1 | 0 | 17.5 | 26.8 |
| | | | | B | 0.9 | 0 | 10.6 | 3.8 |
| | | | | C | 0.7 | 0 | 2.3 | 8.5 |
| | | 2827 | kk-3 | A | 2.5 | 0.7 | 0.8 | 13.4 |
| | | | | B | 2.2 | 0 | 10.5 | 3.9 |
| | | | | C | 0.1 | 0.2 | 6.9 | 34.6 |
| | | 2830 | kk-l | A | 0.7 | 0.2 | 0 | 20.6 |
| | | | | B | 26 | 4 | 0.1 | 3.4 |
| | | | | C | 11.8 | 28.3 | 0.1 | 1.7 |
| | | Transferable in 1/3 systems: | | | | | | |
| | | 2798 | kk-8 | A | 0.1 | 0 | 5.6 | 35.6 |
| | | | | B | 0.7 | 0 | 10.3 | 6.2 |
| | | | | C | 2.3 | 0.2 | 2.3 | 0.9 |

*System A: CAT-2200 H1/L1 & Pertuzumab H2/L2 (with 10:20:24:46 ratio H1:H2:L1:L2 DNA); System B: CAT-2200 H1/L1 & SGN-CD19a H2/L2 (with 15:15:35:35 ratio H1:H2:L1:L2 DNA); System C: CR8071 H1/L1 & SGN-CD19a H2/L2 (with 15:15:35:35 ratio H1:H2:L1:L2 DNA)
**Considering full Ab species only
^{§}Designs arc shaded with increasing grey according to their transferability in 3/3 (not shaded), 2/3, or 1/3 systems, based on a positive Change in % H1L1 and % H2L2 Fairing with respect to wild type
^{‡}Estimated change with respect to wild type

TABLE 17
LC-MS pairing data and post pA yields (mg/L) for K-K-derived K-L designs (total bispecific)

| | | | | | | | | | |
|---|---|---|---|---|---|---|---|---|---|
| | | | | | Change | | Change | | Change |
| | | | | | in % | | in | | in % |
| | | | | | H1L1 | | % H2L2 | | H1L1 and |
| | | | Post | | Pairing | | Pairing | % | % H2L2 |
| | | | pA | | with | | with | H1L1 | Pairing |
| | | | yield | % | respect | % | respect | and % | with |
| | | | (mg/L | H1L1 | to wild | H2L2 | to wild | H2L2 | respect to |
| Design^{§} | Cluster | System* | A280) | Pairing | type | Pairing | type | Pairing | wild type |

Transferable in 3/3 systems:

| | | | | | | | | | |
|---|---|---|---|---|---|---|---|---|---|
| 34 | kk-6 | A | 37 | 85.9 | 34.1^{‡} | 98.8 | 27.7^{‡} | 92.8 | 25.5^{‡} |
| | | B | 56 | 94.6 | 24.5^{‡} | 93.8 | 39.6^{‡} | 94.1 | 27.4^{‡} |
| | | C | 45 | 92.6 | 24.5 | 99.1 | 27.8 | 96.6 | 27.7 |

Transferable in 2/3 systems:

| | | | | | | | | | |
|---|---|---|---|---|---|---|---|---|---|
| 2798 | kk-8 | A | 64 | 89.5 | 37.7^{‡} | 88 | 16.9^{‡} | 88.3 | 21^{‡} |
| | | B | 54 | 94 | 2 | 37.8 | 0.2 | 61.4 | −9.8 |
| | | C | 51 | 97.6 | 15.3 | 34.5 | −25.4 | 66.1 | −5.7 |
| 2816 | kk-4 | A | 37 | 99.6 | 47.8^{‡} | 63.1 | −8^{‡} | 72.1 | 4.8^{‡} |
| | | B | 41 | 94.4 | 2.4 | 25.1 | −12.5 | 55.6 | −15.6 |
| | | C | 47 | 99.3 | 17 | 79.7 | 19.8 | 88.5 | 16.7 |
| 2826 | kk-2 | A | 38 | 23.1 | 4 | 99.6 | 6.5 | 67.8 | 6.8 |
| | | B | 44 | 53.7 | −16.4 | 89.7 | 35.5 | 75.3 | 8.6 |
| | | C | 28 | 27.8 | −40.3 | 98 | 26.7 | 69.6 | 0.7 |
| 2827 | kk-3 | A | 37 | 97.9 | 46.1^{‡} | 93.6 | 22.5^{‡} | 95.5 | 28.2^{‡} |
| | | B | 43 | 98.9 | 6.9 | 22.3 | −15.3 | 56 | −15.2 |
| | | C | 41 | 98.2 | 15.9 | 85 | 25.1 | 88.5 | 16.7 |
| 2830 | kk-1 | A | 24 | 76.8 | 25^{‡} | 99.6 | 28.5^{‡} | 90.9 | 23.6^{‡} |
| | | B | 23 | 83.8 | 13.7^{‡} | 98.5 | 44.3^{‡} | 88.7 | 22^{‡} |
| | | C | 25 | 28 | −20.4 | 99.6 | 20 | 46.5 | −11 |

Transferable in 1/3 systems:

| 2188 | kk-10 | A | 19 | 28.5 | 9.4 | 98 | 4.9 | 69.8 | 8.8 |
|---|---|---|---|---|---|---|---|---|---|
| | | B | 21 | 31 | −39.1 | 92.9 | 38.7 | 54 | −12.7 |
| | | C | 16 | 24.9 | −23.5 | 99.5 | 19.9 | 66.2 | 8.7 |
| | | | | Change in | | | | | |
| | | | | H1L1_H2L2 | | | | | |
| | | | | and | | | | | |
| | | | H1L1_H2L2 | H1L2_H2L1** | H1L1_H2L2 | | | | |
| | | | and | with | and | | | | |
| | | | H1L2_H2L1** | respect to | H1L2_H2L1 | H1L1_H1L1 | H1L1_H1L2 | H1L2_H1L2 | H2L1_H2L1 |
| Design^{§} | Cluster | System* | [symbol] | wild type | [symbol] | [symbol] | [symbol] | [symbol] | [symbol] |

Transferable in 3/3 systems:

| | | | | | | | | | |
|---|---|---|---|---|---|---|---|---|---|
| 34 | kk-6 | A | 82.3 | 50.4^{‡} | 71.9 | 0.6 | 0.2 | 0 | 0.1 |
| | | B | 90.1 | 55^{‡} | 73.3 | 0.1 | 0.1 | 0 | 0.1 |
| | | C | 86.4 | 34.5 | 66.1 | 1 | 1.1 | 0 | 0 |

Transferable in 2/3 systems:

| | | | | | | | | | |
|---|---|---|---|---|---|---|---|---|---|
| 2798 | kk-8 | A | 61.6 | 29.7^{‡} | 36.1 | 0 | 0 | 0 | 0.5 |
| | | B | 33.8 | 1.7 | 28 | 0.2 | 0.9 | 0.9 | 1.6 |
| | | C | 32.4 | −15.7 | 30.6 | 1.2 | 0.1 | 0.2 | 0.3 |
| 2816 | kk-4 | A | 58 | 26.1^{‡} | 32.2 | 0 | 0 | 0.1 | 1 |
| | | B | 23.1 | −9 | 19.5 | 0.4 | 1.4 | 1.7 | 1.3 |
| | | C | 78.5 | 30.4 | 69.4 | 0.2 | 0.3 | 0 | 0.1 |
| 2826 | kk-2 | A | 20.5 | 3.6 | 16.5 | 0.4 | 0.1 | 0 | 0.1 |
| | | B | 48.3 | 13.2 | 38.8 | 0 | 0.1 | 0.1 | 0.4 |
| | | C | 25.9 | −26 | 20.4 | 0.2 | 0.7 | 1 | 0 |
| 2827 | kk-3 | A | 90.8 | 58.9^{‡} | 75 | 0.3 | 0 | 0 | 0.8 |
| | | B | 19.1 | −13 | 15.9 | 0.5 | 0.5 | 0.1 | 0.3 |
| | | C | 76.2 | 28.1 | 44.4 | 0.1 | 0.2 | 0.1 | 0.1 |
| 2830 | kk-1 | A | 72.2 | 40.3^{‡} | 56.6 | 0.1 | 0.1 | 0 | 0.2 |
| | | B | 74.7 | 39.6^{‡} | 49.7 | 3.7 | 1.5 | 1.4 | 0.1 |
| | | C | 22 | −24.8 | 12.8 | 0.7 | 3.7 | 5.7 | 0 |

Transferable in 1/3 systems:

| 2188 | kk-10 | A | 27.3 | 10.4 | 22 | 0 | 0.1 | 0.1 | 0.2 |
|---|---|---|---|---|---|---|---|---|---|
| | | B | 31.2 | −3.9 | 21.1 | 0.4 | 0.9 | 1.8 | 0.1 |
| | | C | 24.6 | −22.2 | 21.3 | 0.1 | 0.4 | 1.5 | 0 |
| | | | H2L1_H2L2 | H2L2_H2L2 | H1L1_H2L1 | H1L2_H2L2 | H1L1 | H1L2 | H2L1 |
| Design^{§} | Cluster | System* | [symbol] | [symbol] | [symbol] | [symbol] | [symbol] | [symbol] | [symbol] |

Transferable in 3/3 systems:

| | | | | | | | | | |
|---|---|---|---|---|---|---|---|---|---|
| 34 | kk-6 | A | 0.2 | 1.9 | 0.6 | 11.8 | 2.5 | 0.5 | 0.1 |
| | | B | 0.1 | 2 | 1.6 | 4.1 | 0.4 | 0.1 | 2.8 |
| | | C | 0.1 | 3.4 | 0.3 | 4.4 | 0 | 0 | 0.4 |

Transferable in 2/3 systems:

| | | | | | | | | | |
|---|---|---|---|---|---|---|---|---|---|
| 2798 | kk-8 | A | 1.7 | 10.9 | 4.6 | 4.8 | 0.1 | 0 | 5.6 |
| | | B | 0.2 | 0.5 | 48.2 | 2.3 | 0.7 | 0 | 10.3 |
| | | C | 0.1 | 0.2 | 60 | 1.4 | 2.3 | 0.2 | 2.3 |
| 2816 | kk-4 | A | 1.8 | 3.6 | 16.8 | 0 | 0.1 | 0 | 17.5 |
| | | B | 0.2 | 0.2 | 59.7 | 0.2 | 0.9 | 0 | 10.6 |
| | | C | 0.2 | 0.6 | 17.4 | 0.2 | 0.7 | 0 | 2.3 |
| 2826 | kk-2 | A | 0.1 | 2.4 | 0 | 61 | 0.9 | 1.4 | 0 |
| | | B | 0.2 | 1.3 | 3.6 | 35.9 | 0.2 | 0.4 | 4 |
| | | C | 0 | 1.3 | 0.2 | 55.1 | 0.4 | 0.4 | 1.1 |
| 2827 | kk-3 | A | 0 | 2.2 | 4 | 0.4 | 2.5 | 0.7 | 0.8 |
| | | B | 0.1 | 0.4 | 65.3 | 0.2 | 2.2 | 0 | 10.5 |
| | | C | 0.8 | 5.2 | 7.3 | 0.2 | 0.1 | 0.2 | 6.9 |
| 2830 | kk-1 | A | 0.1 | 4.2 | 0 | 17.1 | 0.7 | 0.2 | 0 |
| | | B | 0 | 0.2 | 0.7 | 9.2 | 26 | 4 | 0.1 |
| | | C | 0 | 0 | 0 | 35.2 | 11.8 | 28.3 | 0.1 |

Transferable in 1/3 systems:

| 2188 | kk-10 | A | 0.6 | 3.5 | 0.3 | 53.6 | 0.4 | 2.1 | 0.5 |
|---|---|---|---|---|---|---|---|---|---|
| | | B | 0 | 0.1 | 3.1 | 40.3 | 6.6 | 20.9 | 0.9 |
| | | C | 0 | 1 | 0.1 | 62.2 | 0.1 | 0.7 | 0.2 |
| | | | | | | | | | H2L2 |
| | | | | | | Design^{§} | Cluster | System* | [symbol] |
| | | | | | | Transferable in 3/3 systems: | | | |
| | | | | | | 34 | kk-6 | A | 9.5 |
| | | | | | | | | B | 15.4 |
| | | | | | | | | C | 23 |
| | | | | | | Transferable in 2/3 systems: | | | |
| | | | | | | 2798 | kk-8 | A | 35.6 |
| | | | | | | | | B | 6.2 |
| | | | | | | | | C | 0.9 |
| | | | | | | 2816 | kk-4 | A | 26.8 |
| | | | | | | | | B | 3.8 |
| | | | | | | | | C | 8.5 |
| | | | | | | 2826 | kk-2 | A | 17 |
| | | | | | | | | B | 15.1 |
| | | | | | | | | C | 19.3 |
| | | | | | | 2827 | kk-3 | A | 13.4 |
| | | | | | | | | B | 3.9 |
| | | | | | | | | C | 34.6 |
| | | | | | | 2830 | kk-1 | A | 20.6 |
| | | | | | | | | B | 3.4 |
| | | | | | | | | C | 1.7 |
| | | | | | | Transferable in 1/3 systems: | | | |
| | | | | | | 2188 | kk-10 | A | 16.6 |
| | | | | | | | | B | 3.7 |
| | | | | | | | | C | 12.3 |

*System A: CAT-2200 H1/L1 & Pertuzumab H2/L2 (with 10:20:24:46 ratio H1:H2:L1:L2 DNA); System B: CAT-2200 H1/L1 & SGN-CD19a H2/L2 (with 15:15:35:35 ratio H1:H2:L1:L2 DNA); System C: CR8071 H1/L1 & SGN-CD19a H2/L2 (with 15:15:35:35 ratio H1:H2:L1:L2 DNA)
**Considering full Ab species only
^{§}Designs arc shaded with increasing grey according to their transferability in 3/3 (not shaded), 2/3, or 1/3 systems, based on a positive Change in H1L1_H2L2 and H1L2_H2L1** with respect to wild type
^{‡}Estimated change with respect to wild type

TABLE 18
Post prep-SEC LC-MS pairing data for selected designs in three systems

| | | | | Change | | Change | | Change | |
|---|---|---|---|---|---|---|---|---|---|
| | | | | in % | | in | | in % | |
| | | | | H1L1 | | % H2L2 | | H1L1 and | |
| | | | | Pairing | | Pairing | % | % H2L2 | |
| | | | | with | | with | H1L1 | Pairing | H1L1_H2L2 |
| | | | % | respect | % | respect | and % | with | and |
| | | | H1L1 | to wild | H2L2 | to wild | H2L2 | respect to | H1L2_H2L1** |
| Design | Cluster | System* | Pairing | type | Pairing | type | Pairing | wild type | [symbol] |
| 2901 | kl-6 | A | 95.4 | 76.3 | 98.6 | 5.5 | 97.3 | 36.3 | 90.8 |
| | | B | 88.1 | 18 | 95.7 | 41.5 | 92.1 | 25.4 | 84.7 |
| | | C | 63.3 | 14.9 | 99.8 | 20.2 | 81.9 | 24.4 | 62.9 |
| 3972 | kl-2 | A | 100 | 48.2^{‡} | 98.4 | 27.3^{‡} | 99.2 | 31.9^{‡} | 98.7 |
| | | B | 99.2 | 29.1^{‡} | 97.7 | 43.5^{‡} | 98.5 | 31.8^{‡} | 92.7 |
| | | C | 92.3 | 43.9 | 99.9 | 20.3 | 96.1 | 38.6 | 88.5 |
| | | | Change in | | | | | | |
| | | | H1L1_H2L2 | | | | | | |
| | | | and | | | | | | |
| | | | H1L2_H2L1** | H1L1_H2L2 | | | | | |
| | | | with | and | | | | | |
| | | | respect to | H1L2_H2L1 | H1L1_H1L1 | H1L1_H1L2 | H1L2_H1L2 | H2L1_H2L1 | H2L1_H2L2 |
| Design | Cluster | System* | wild type | [symbol] | [symbol] | [symbol] | [symbol] | [symbol] | [symbol] |
| 2901 | kl-6 | A | 73.9 | 74.1 | 0 | 0.1 | 0.1 | 0.1 | 1.2 |
| | | B | 49.6 | 76.1 | 0.3 | 0.3 | 0.2 | 0.2 | 0 |
| | | C | 16.1 | 56.5 | 0.3 | 1 | 1 | 0 | 0 |
| 3972 | kl-2 | A | 66.8^{‡} | 86.5 | 0.2 | 0 | 0 | 0.8 | 0.1 |
| | | B | 57.6^{‡} | 83.7 | 4.9 | 0.1 | 0 | 0.1 | 0 |
| | | C | 41.7 | 82.3 | 3.2 | 1 | 0.1 | 0 | 0 |
| | | | H2L2_H2L2 | H1L1_H2L1 | H1L2_H2L2 | H1L1 | H1L2 | H2L1 | H2L2 |
| Design | Cluster | System* | [symbol] | [symbol] | [symbol] | [symbol] | [symbol] | [symbol] | [symbol] |
| 2901 | kl-6 | A | 2.3 | 0.2 | 3.5 | 2.7 | 0.1 | 0 | 15.5 |
| | | B | 0 | 2.8 | 9.8 | 2.3 | 0.5 | 0.6 | 6.9 |
| | | C | 0 | 0 | 30.9 | 2 | 1.1 | 0 | 7.1 |
| 3972 | kl-2 | A | 0.1 | 0 | 0 | 5.9 | 0 | 0 | 6.5 |
| | | B | 0 | 0.8 | 0.6 | 3.3 | 0 | 0.6 | 5.7 |
| | | C | 0 | 0 | 6.2 | 1.7 | 0.2 | 0 | 5.2 |

*System A CAT-2200 H1/L1 & Pertuzumab H2/L2 (with 10:20:24:46 ratio H1:H2:L1:L2 DNA); System B: CAT-2200 H1/L1 & SGN-CD19a H2/L2 (with 15:15:35:35 ratio H1:H2:L1:L2 DNA); System C: CR8071 H1/L1 & SGN-CD19a H2/L2 (with 15:15:35:35 ratio H1:H2:L1:L2 DNA)
**Considering full Ab species only
^{‡}Estimated change with respect to wild type

TABLE 19
Antigen binding data for wild type paratopes in OAA format

| | | | | Median | Mean | Standard |
|---|---|---|---|---|---|---|
| | | | | KD | KD | Deviation |
| Warhead | Tag | Antigen | Count | (nM) | (nM) | (nM) |
| CAT-2200 | NO TAGS | IL-17A | 6 | 0.282 | 0.287 | 0.040 |
| CAT-2200 | /FLAG | IL-17A | 6 | 0.375 | 0.366 | 0.063 |
| CAT-2200 | /HA | IL-17A | 6 | 0.373 | 0.350 | 0.061 |
| CR8071 | NO TAGS | HEMAGGLUTININ | 3 | 0.385 | 0.379 | 0.013 |
| CR8071 | /FLAG | HEMAGGLUTININ | 3 | 0.332 | 0.332 | 0.007 |

| | | | | | |
|---|---|---|---|---|---|
| Pertuzumab | /FLAG | HERZ | 1 | 7.010 | NA |
| Pertuzumab | NO TAGS | HERZ | 1 | 3.710 | NA |
| SGN-CD19a | NO TAGS | CD19 | 2 | 67.535 | 6.951 |
| SGN-CD19a* | NO TAGS | CD19 | 1 | 71.280 | NA |
| SGN-CD19a* | /HA | CD19 | 1 | 137.000 | NA |
| SGN-CD19a | /HA | CD19 | 2 | 150.100 | 1.273 |
| SGN-CD19a | /FLAG | CD19 | 2 | 224.550 | 22.981 |
| SGN-CD19a* | /FLAG | CD19 | 1 | 321.500 | NA |

*Denotes a different capture method

TABLE 20
Antigen binding affinity and thermal stability assessment for select K-L designs

| | | | | | | | | | Average |
|---|---|---|---|---|---|---|---|---|---|
| | | | | | | | | | difference |
| | | | | | | | | | in Fab Tm |
| | | | | Difference | | Difference | | | (across |
| | | | | in H1L1 | | in H2L2 | Tm | Tm | systems) |
| | | | H1L1 | log(KD) | H2L2 | log(KD) | H1L1 | H2L2 | from |
| | | | KD | from | KD | from | Fab | Fab | wild type |
| Design^{§} | Cluster | System* | (nM) | wild type | (nM) | wild type | (° C.)†† | (° C.)†† | (° C.)‡‡ |
| 2979 | kl-4 | A | 0.31 | −0.03 | 3.06 | 0.08 | 70.4 | 74.0 | −0.4 |
| | | B | 0.39 | −0.14 | 148.80 | 0.18 | 70.1 | 91.4 | |
| | | C | 0.93 | −0.38 | 45.06 | 0.70 | 68.5 | 91.5 | |
| 3018 | kl-3 | A | 0.53 | −0.16 | 2.89 | 0.11 | 69.9 | 74.3 | −1.4 |
| | | B | 0.35 | 0.03 | 47.97 | 0.15 | 69.5 | 88.8 | |
| | | C | 0.16 | 0.39 | 93.89 | −0.14 | 67.1 | 89.2 | |
| 3041 | kl-3 | A | ND† | ND | ND | ND | ND | ND | −0.5 |
| | | B | 0.42 | −0.06 | MVB‡, 62.0** | MVB, 0.11** | 69.6 | 91.7 | |
| | | C | ND | ND | ND | ND | ND | ND | |
| 3102 | kl-12 | A | 0.37 | −0.12 | 3.14 | 0.07 | 71.1 | 71.1 | −1.0 |
| | | B | 0.41 | −0.16 | 215.20 | 0.17 | 70.4 | 91.8 | |
| | | C | 0.87 | −0.35 | MVB | MVB | 67.8 | 91.7 | |
| 3898 | kl-8 | A | 0.33 | −0.06 | 2.87 | 0.11 | 72.2 | 74.0 | −0.4 |
| | | B | 0.44 | −0.19 | 188.10 | 0.23 | 71.6 | 89.6 | |
| | | C | 0.50 | −0.12 | 220.60 | 0.16 | 68.3 | 89.8 | |
| 3947 | kl-4 | A | 0.31 | −0.04 | 3.28 | 0.05 | 70.9 | 73.1 | −1.4 |
| | | B | 0.36 | −0.10 | MVB, 65.4** | MVB, 0.09** | 70.4 | 88.0 | |
| | | C | 1.14 | −0.47 | MVB, 65.4** | MVB, 0.09** | 68.3 | 87.8 | |
| 3025 | kl-2 | A | 0.47 | −0.10 | 3.28 | 0.05 | 70.6 | 72.1 | −2.6 |
| | | B | 0.45 | −0.08 | MVB | MVB | 69.4 | 83.8 | |
| | | C | 0.68 | −0.31 | 61.05 | 0.07 | 68.9 | 84.4 | |
| 3109 | kl-8 | A | 0.37 | −0.12 | 2.96 | 0.10 | 71.6 | 71.6 | −3.0 |
| | | B | 0.47 | −0.22 | MVB, 140** | MVB, 0.06** | 70.5 | 83.5 | |
| | | C | 1.50 | −0.59 | MVB, 140** | MVB, 0.06** | 67.4 | 83.7 | |
| 3113 | kl-12 | A | 0.39 | −0.14 | 3.05 | 0.09 | 70.6 | 70.6 | −1.6 |
| | | B | 0.44 | −0.20 | 216.00 | 0.02 | 70.0 | 91.7 | |
| | | C | 0.99 | −0.41 | MVB | MVB | 66.8 | 91.6 | |
| 3878 | kl-8 | A | 0.36 | −0.10 | 3.03 | 0.09 | 73.5 | 73.5 | −1.6 |
| | | B | ND | ND | ND | ND | ND | ND | |
| | | C | 0.32 | 0.08 | 306.30 | −0.14 | 68.3 | 83.9 | |
| 3890 | kl-9 | A | 0.44 | −0.19 | 2.80 | 0.12 | 73.2 | 73.2 | −1.9 |
| | | B | 0.44 | −0.19 | 54.83 | 0.09 | 71.4 | 83.7 | |
| | | C | 0.44 | −0.06 | 40.94 | 0.22 | 68.5 | 84.3 | |
| 3910 | kl-10 | A | 0.37 | −0.11 | 2.88 | 0.11 | 71.3 | 71.3 | −2.7 |
| | | B | ND | ND | ND | ND | ND | ND | |
| | | C | 0.47 | −0.09 | 294.00 | −0.12 | 68.2 | 83.9 | |
| 3931 | kl-7 | A | 0.44 | −0.07 | 2.90 | 0.11 | 69.7 | 73.7 | −1.6 |
| | | B | 0.29 | 0.11 | 54.93 | 0.09 | 69.5 | 87.2 | |
| | | C | 0.45 | −0.13 | 64.37 | 0.04 | 68.6 | 87.1 | |
| 3954 | kl-7 | A | 0.34 | −0.09 | 3.10 | 0.08 | 71.8 | 72.9 | −2.2 |
| | | B | 0.26 | 0.04 | 76.38 | −0.03 | 70.6 | 83.6 | |
| | | C | 0.30 | 0.04 | 70.76 | 0.00 | 68.4 | 84.8 | |
| 3967 | kl-9 | A | 0.37 | −0.11 | 5.29 | 0.12 | 70.0 | 70.0 | −2.9 |
| | | B | 0.38 | −0.13 | 36.12 | 0.27 | 69.8 | 86.1 | |
| | | C | 0.52 | −0.13 | 43.33 | 0.19 | 68.0 | 86.6 | |
| 4010 | kl-12 | A | 0.42 | −0.17 | 5.41 | 0.11 | 71.4 | 71.4 | −2.9 |
| | | B | 0.36 | −0.11 | 131.70 | 0.23 | 70.3 | 83.2 | |
| | | C | 0.49 | −0.10 | 227.50 | −0.01 | 68.2 | 84.1 | |
| 4040 | kl-8 | A | 0.33 | −0.06 | 2.30 | 0.21 | 71.5 | 72.5 | −2.0 |
| | | B | 0.31 | −0.04 | 16.97, 22.4** | 0.62, 0.55** | 70.7 | 84.0 | |
| | | C | 0.66 | −0.24 | 3.53, 22.4** | 1.30, 0.55** | 69.1 | 86.2 | |
| 2892 | kl-6 | A | 0.65 | −0.24 | 3.24 | 0.06 | 67.7 | 74.8 | −3.6 |
| | | B | 0.53 | −0.15 | 49.74 | 0.13 | 65.8 | 84.1 | |
| | | C | 0.95 | −0.46 | 39.69 | 0.25 | 65.6 | 84.3 | |
| 2901 | kl-6 | A | 0.54 | −0.16 | 3.12 | 0.08 | 67.1 | 71.5 | −3.4 |
| | | B | 0.52 | −0.14 | 47.05 | 0.16 | 66.2 | 88.1 | |
| | | C | 0.64 | −0.29 | 41.91 | 0.21 | 66.1 | 87.6 | |
| 2994 | kl-3 | A | 0.45 | −0.08 | 3.20 | 0.06 | 69.0 | 73.5 | −4.3 |
| | | B | 0.28 | 0.12 | 49.69 | 0.16 | 68.3 | 83.7 | |
| | | C | ND | ND | ND | ND | ND | ND | |
| 3008 | kl-3 | A | 0.35 | 0.02 | 3.10 | 0.08 | 69.4 | 69.4 | −4.0 |
| | | B | 0.31 | 0.08 | 41.12 | 0.21 | 68.8 | 83.7 | |
| | | C | 0.47 | −0.15 | 48.01 | 0.15 | 67.5 | 83.9 | |
| 3122 | kl-5 | A | ND | ND | ND | ND | ND | ND | −4.1 |
| | | B | 0.43 | −0.19 | 195.60 | 0.06 | 69.9 | 80.1 | |
| | | C | 0.84 | −0.34 | MVB | MVB | 67.0 | 79.8 | |
| 3951 | kl-4 | A | 0.36 | −0.11 | 2.98 | 0.10 | 69.9 | 69.9 | −3.4 |
| | | B | 0.34 | −0.08 | 42.33 | 0.20 | 69.7 | 84.2 | |
| | | C | 0.55 | −0.16 | 38.35 | 0.24 | 68.1 | 84.3 | |
| 3957 | kl-7 | A | 0.37 | 0.00 | 2.83 | 0.12 | 69.4 | 69.4 | −3.8 |
| | | B | 0.24 | 0.19 | 54.58 | 0.09 | 69.0 | 82.8 | |
| | | C | 0.32 | 0.02 | 56.65 | 0.08 | 68.4 | 83.8 | |
| 3971 | kl-2 | A | 0.47 | −0.10 | 3.02 | 0.09 | 70.8 | 70.8 | −4.4 |
| | | B | 0.47 | −0.10 | 37.65 | 0.25 | 70.1 | 78.8 | |
| | | C | 0.40 | −0.08 | 53.08 | 0.10 | 68.5 | 78.3 | |
| 3972 | kl-2 | A | 0.32 | −0.05 | 3.11 | 0.08 | 71.9 | 71.9 | −3.3 |
| | | B | 0.29 | −0.02 | 53.63 | 0.12 | 70.9 | 80.2 | |
| | | C | 0.38 | −0.06 | 30.18 | 0.37 | 68.6 | 80.6 | |
| 3974 | kl-1 | A | 0.46 | −0.09 | 2.92 | 0.10 | 70.9 | 70.9 | −3.9 |
| | | B | 0.27 | 0.13 | 50.95 | 0.12 | 69.9 | 81.6 | |
| | | C | 0.44 | −0.12 | 62.40 | 0.03 | 67.5 | 81.0 | |
| 3980 | kl-2 | A | 0.32 | −0.05 | 2.82 | 0.12 | 71.1 | 71.1 | −4.9 |
| | | B | 0.25 | 0.05 | 55.39 | 0.08 | 70.9 | 82.0 | |
| | | C | ND | ND | ND | ND | ND | ND | |
| 3987 | kl-3 | A | 0.30 | −0.02 | 3.22 | 0.06 | 70.9 | 70.9 | −3.9 |
| | | B | 0.23 | 0.08 | 48.45 | 0.17 | 70.6 | 80.1 | |
| | | C | 0.23 | 0.16 | 63.74 | 0.05 | 68.4 | 80.3 | |
| 3997 | kl-1 | A | 0.33 | −0.07 | 2.95 | 0.10 | 69.7 | 69.7 | −4.2 |
| | | B | ND | ND | ND | ND | ND | ND | |
| | | C | ND | ND | ND | ND | ND | ND | |
| 4008 | kl-8 | A | 0.43 | −0.18 | 5.57 | 0.10 | 70.7 | 70.7 | −3.2 |
| | | B | 0.39 | −0.14 | 217.50 | 0.01 | 69.8 | 83.5 | |
| | | C | 0.48 | −0.09 | 272.90 | −0.09 | 68.0 | 84.6 | |
| 4320 | kl-10 | A | 0.36 | −0.10 | 2.90 | 0.11 | 69.9 | 69.9 | −3.5 |
| | | B | 0.38 | −0.13 | 306.70 | −0.14 | 69.9 | 83.3 | |
| | | C | 0.49 | −0.11 | 257.90 | −0.06 | 67.9 | 84.2 | |

*System A: CAT-2200 H1/L1 & Pertuzumab H2/L2 (with 10:20:24:46 ratio H1:H2:L1:L2 DNA); System B: CAT-2200 H1/L1 & SGN-CD19a H2/L2 (with 15:15:35:35 ratio H1:H2:L1:L2 DNA); System C: CR8071 H1/L1 & SGN-CD19a H2/L2 (with 15:15:35:35 ratio H1:H2:L1:L2 DNA)
^{§}Designs are grouped and shaded in three bins with “Average difference in Fab Tm from wild type (° C.)” from 0 to −1.5, −1.5 to −3.0, and less than −3.
†ND = Not determined
‡MVB = Mixed valency binding in SMCA format
**KD determined in OAA format
††DSC for antibodies was performed in SMCA format, and thermal transitions attributable to each Fab arm are given.
‡‡Wild type Fab Tms were obtained from DSC experiments in Mab format

TABLE 21
Antigen binding affinity and thermal stability assessment for select K-K-derived K-L designs

| | | | | | | | | | Average |
|---|---|---|---|---|---|---|---|---|---|
| | | | | | | | | | difference |
| | | | | Difference | | Difference | | | in Fab Tm |
| | | | | in H1L1 | | in H2L2 | | | (across |
| | | | H1L1 | log(KD) | H2L2 | log(KD) | Tm | Tm | systems) from |
| | | | KD | from | KD | from | H1L1 | H2L2 | wild type |
| Design^{§} | Cluster | System* | (nM) | wild type | (nM) | wild type | (° C.) †† | (° C.) †† | (° C.) ‡‡ |
| 34 | kk-6 | A | 0.63 | −0.35 | 2.63 | 0.15 | 69.6 | 72.9 | −1.5 |
| | | B | 0.63 | −0.35 | 17.07 | 0.60 | 68.3 | 90.3 | |
| | | C | 0.66 | −0.23 | 24.18 | 0.44 | 67.2 | 90.9 | |
| 2827 | kk-3 | A | 0.38 | −0.13 | 2.93 | 0.10 | 72.0 | 73.1 | −1.2 |
| | | B | ND† | ND | ND | ND | ND | ND | |
| | | C | 0.72 | −0.27 | 111.90 | 0.30 | 68.8 | 86.9 | |
| 2816 | kk-4 | A | 0.51 | −0.26 | 3.08 | 0.08 | 72.5 | 72.5 | −2.3 |
| | | B | ND | ND | ND | ND | ND | ND | |
| | | C | 0.42 | −0.04 | 281.40 | 0.06 | 68.2 | 83.3 | |
| 2830 | kk-1 | A | 0.45 | −0.20 | 3.08 | 0.08 | 70.1 | 73.7 | −3.7 |
| | | B | 0.32 | −0.06 | 50.84 | 0.15 | 69.3 | 84.3 | |
| | | C | ND | ND | ND | ND | ND | ND | |

*System A: CAT-2200 H1/L1 & Pertuzumab H2/L2 (with 10:20:24:46 ratio H1:H2:L1:L2 DNA); System B: CAT-2200 H1/L1 & SGN-CD19a H2/L2 (with 15:15:35:35 ratio H1:H2:L1:L2 DNA); System C: CR8071 H1/L1 & SGN-CD19a H2/L2 (with 15:15:35:35 ratio H1:H2:L1:L2 DNA)
^{§}Designs are grouped and shaded in three bins with “Average difference in Fab Tm from wild type (° C.)” from 0 to −1.5, −1.5 to −3.0, and less than −3.
†ND = Not determined
†† DSC for antibodies was performed in SMCA format, and thermal transitions attributable to each Fab arm are given.
‡‡ Wild type Fab Tms were obtained from DSC experiments in Mab format

TABLE 22A
IgG1 heavy chain amino acid residue numbering according
to IMGT, Kabat, 1JPT, and EU numbering systems

| IMGT | Kabat | 1JPT | EU |
|---|---|---|---|
| 1044 | 39 | 39 | Not |
| | | | applicable |
| 1050 | 45 | 45 | Not |
| | | | applicable |
| 3005 | 122 | 126 | 126 |
| 3007 | 124 | 128 | 128 |
| 3008 | 125 | 129 | 129 |
| 3020 | 139 | 141 | 141 |
| 3024 | 143 | 145 | 145 |
| 3026 | 145 | 147 | 147 |
| 3027 | 146 | 148 | 148 |
| 3081 | 174 | 170 | 170 |
| 3084 | 177 | 173 | 173 |
| 3084B | 179 | 175 | 175 |
| 3085Y | 186 | 181 | 181 |
| 3086 | 188 | 183 | 183 |
| 3088 | 190 | 185 | 185 |
| 4003 | 228 | 218 | 218 |

TABLE 22B
Lambda light chain amino acid residue numbering
according to IMGT, and Kabat numbering systems

| IMGT | Kabat |
|---|---|
| 1044 | 38 |
| 3005 | 116 |
| 3011 | 122 |
| 3013 | 124 |
| 3018 | 129 |
| 3020 | 131 |
| 3022 | 133 |
| 3024 | 135 |
| 3086 | 176 |
| 3088 | 178 |
| 3090 | 180 |

TABLE 22C
Kappa light chain amino acid residue numbering
according to IMGT, Kabat, and 1JPT numbering systems

| IMGT | Kabat | 1JPT |
|---|---|---|
| 1044 | 38 | 38 |
| 1050 | 44 | 44 |
| 3005 | 116 | 116 |
| 3010 | 121 | 121 |
| 3013 | 124 | 124 |
| 3018 | 129 | 129 |
| 3020 | 131 | 131 |
| 3022 | 133 | 133 |
| 3024 | 135 | 135 |
| 3079 | 160 | 160 |
| 3086 | 176 | 176 |
| 3088 | 178 | 178 |
| 3090 | 180 | 180 |

TABLE 23
Correspondence table for
% Paired/% Mispaired species and LCCA Scalar
Values

| Paired species (%) | Mispaired species (%) | Icca_scalar |
|---|---|---|
| 99 | 1 | 4.60 |
| 98 | 2 | 3.89 |
| 97 | 3 | 3.48 |
| 96 | 4 | 3.18 |
| 95 | 5 | 2.94 |
| 94 | 6 | 2.75 |
| 93 | 7 | 2.59 |
| 92 | 8 | 2.44 |
| 91 | 9 | 2.31 |
| 90 | 10 | 2.20 |
| 89 | 11 | 2.09 |
| 88 | 12 | 1.99 |
| 87 | 13 | 1.90 |
| 86 | 14 | 1.82 |
| 85 | 15 | 1.73 |
| 84 | 16 | 1.66 |
| 83 | 17 | 1.59 |
| 82 | 18 | 1.52 |
| 81 | 19 | 1.45 |
| 80 | 20 | 1.39 |
| 79 | 21 | 1.32 |
| 78 | 22 | 1.27 |
| 77 | 23 | 1.21 |
| 76 | 24 | 1.15 |
| 75 | 25 | 1.10 |
| 74 | 26 | 1.05 |
| 73 | 27 | 0.99 |
| 72 | 28 | 0.94 |
| 71 | 29 | 0.90 |
| 70 | 30 | 0.85 |
| 69 | 31 | 0.80 |
| 68 | 32 | 0.75 |
| 67 | 33 | 0.71 |
| 66 | 34 | 0.66 |
| 65 | 35 | 0.62 |
| 64 | 36 | 0.58 |
| 63 | 37 | 0.53 |
| 62 | 38 | 0.49 |
| 61 | 39 | 0.45 |
| 60 | 40 | 0.41 |
| 59 | 41 | 0.36 |
| 58 | 42 | 0.32 |
| 57 | 43 | 0.28 |
| 56 | 44 | 0.24 |
| 55 | 45 | 0.20 |
| 54 | 46 | 0.16 |
| 53 | 47 | 0.12 |
| 52 | 48 | 0.08 |
| 51 | 49 | 0.04 |
| 50 | 50 | 0.00 |
| 49 | 51 | −0.04 |
| 48 | 52 | −0.08 |
| 47 | 53 | −0.12 |
| 46 | 54 | −0.16 |
| 45 | 55 | −0.20 |
| 44 | 56 | −0.24 |
| 43 | 57 | −0.28 |
| 42 | 58 | −0.32 |
| 41 | 59 | −0.36 |
| 40 | 60 | −0.41 |
| 39 | 61 | −0.45 |
| 38 | 62 | −0.49 |
| 37 | 63 | −0.53 |
| 36 | 64 | −0.58 |
| 35 | 65 | −0.62 |
| 34 | 66 | −0.66 |
| 33 | 67 | −0.71 |
| 32 | 68 | −0.75 |
| 31 | 69 | −0.80 |
| 30 | 70 | −0.85 |
| 29 | 71 | −0.90 |
| 28 | 72 | −0.94 |
| 27 | 73 | −0.99 |
| 26 | 74 | −1.05 |
| 25 | 75 | −1.10 |
| 24 | 76 | −1.15 |
| 23 | 77 | −1.21 |
| 22 | 78 | −1.27 |
| 21 | 79 | −1.32 |
| 20 | 80 | −1.39 |
| 19 | 81 | −1.45 |
| 18 | 82 | −1.52 |
| 17 | 83 | −1.59 |
| 16 | 84 | −1.66 |
| 15 | 85 | −1.73 |
| 14 | 86 | −1.82 |
| 13 | 87 | −1.90 |
| 12 | 88 | −1.99 |
| 11 | 89 | −2.09 |
| 10 | 90 | −2.20 |
| 9 | 91 | −2.31 |
| 8 | 92 | −2.44 |
| 7 | 93 | −2.59 |
| 6 | 94 | −2.75 |
| 5 | 95 | −2.94 |
| 4 | 96 | −3.18 |
| 3 | 97 | −3.48 |
| 2 | 98 | −3.89 |
| 1 | 99 | −4.60 |

TABLE 24
SMCA design numbers and
corresponding LCCA set unique identifiers

| SMCA Design ID | LCCA Unique identifier |
|---|---|
| 34 | 10681-10741 |
| 2188 | 10640-10713 |
| 2798 | 10621-10733 |
| 2816 | 10665-10724 |
| 2826 | 10652-10734 |
| 2827 | 10685-10726 |
| 2830 | 10657-10760 |
| 2892 | 10808-11308 |
| 2901 | 10814-11233 |
| 2979 | 11100-11205 |
| 2994 | 10983-11306 |
| 3008 | 10987-11257 |
| 3018 | 10931-11263 |
| 3025 | 10945-11377 |
| 3041 | 10947-11392 |
| 3102 | 11034-11204 |
| 3109 | 11026-11270 |
| 3113 | 11065-11206 |
| 3122 | 11066-11181 |
| 3878 | 12938-13469 |
| 3890 | 13194-13427 |
| 3898 | 13316-13451 |
| 3910 | 13238-13463 |
| 3931 | 12878-11240 |
| 3947 | 13335-13361 |
| 3951 | 13209-13364 |
| 3954 | 13167-13514 |
| 3957 | 13263-13493 |
| 3967 | 13208-13455 |
| 3971 | 13282-13484 |
| 3972 | 13218-13482 |
| 3974 | 13175-13486 |
| 3980 | 13287-13494 |
| 3987 | 13221-13411 |
| 3997 | 13180-13515 |
| 4008 | 13226-13434 |
| 4010 | 13227-13383 |
| 4022 | 13306-13375 |
| 4040 | 13320-13370 |
| 4320* | 13304-13466 |

*design was identical to LCCA design 13304-13466, except that Q124 position in L2 was tested as Q124

1-44. (canceled)

45. An antigen-binding polypeptide construct comprising a first heterodimer and a second heterodimer,
the first heterodimer (H1L1) comprising a first IgG heavy chain polypeptide sequence (H1), and a lambda light chain polypeptide sequence (L1) that form a first Fab region that specifically binds to a first antigen; and the second heterodimer (H2L2) comprising a second IgG heavy chain polypeptide sequence (H2), and a kappa light chain polypeptide sequence (L2) that form a second Fab region that specifically binds to a second antigen,
wherein:
H1 is distinct from H2, and H1 and H2 each comprise a heavy chain variable domain (VH domain), a heavy chain constant domain 1 (CH1 domain), and an IgG hinge region;
L1 comprises a lambda light chain variable (VL-lambda) domain and a lambda light chain constant (CL-lambda) domain, and L2 comprises a kappa light chain variable (VL-kappa) domain and a kappa light chain constant (CL-kappa) domain;
H1, H2, L1, and/or L2 comprise amino acid substitutions that promote preferential pairing of H1 with L1 as compared to L2, and/or that promote preferential pairing of H2 with L2 as compared to L1, at positions identified according to the Kabat numbering system; and
a) H1 comprises amino acid substitutions 125R and 143K, L1 comprises amino acid substitutions 122D, 129T, and 133D, H2 comprises amino acid substitutions 143E, 145T, 179E, and 228D, and L2 comprises amino acid substitutions 121K, 124R, and 178R;
b) H1 comprises amino acid substitutions 125R and 188K, L1 comprises amino acid substitutions 122D, 129T, 176D, and 178T, H2 comprises amino acid substitutions 143E, 145T, 179E, and 228D, and L2 comprises amino acid substitutions 121K, 124R, and 178R;
c) H1 comprises amino acid substitutions 125R and 179K, L1 comprises amino acid substitutions 122D, 129T, and 180E, H2 comprises amino acid substitutions 145T, 179E, and 228D, and L2 comprises amino acid substitutions 121K, 129K, and 131K;
d) H1 comprises amino acid substitutions 125R and 179K, L1 comprises amino acid substitutions 122D, 129T, and 180E, H2 comprises amino acid substitutions 143E, 145T, and 179E, and L2 comprises amino acid substitutions 124R, 135W, 160K, and 178R;
e) H1 comprises amino acid substitutions 125R and 179K, L1 comprises amino acid substitutions 122D, 129T, and 180E, H2 comprises amino acid substitutions 143E, 145T, 179E, and 228D, and L2 comprises amino acid substitutions 121K, 124R, 160K, and 178R;
f) H1 comprises amino acid substitutions 125R and 186R, L1 comprises amino acid substitutions 122D, 129T, and 180E, H2 comprises amino acid substitutions 143E and 145T, and L2 comprises amino acid substitutions 124R, 135W, 160K, and 178R;
g) H1 comprises amino acid substitutions 125R and 186R, L1 comprises amino acid substitutions 122D, 129T, and 180E, H2 comprises amino acid substitutions 143E, 145T, and 228D, and L2 comprises amino acid substitutions 121K, 124R, 160K, and 178R;
h) H1 comprises amino acid substitutions 125R and 186R, L1 comprises amino acid substitutions 122D, 129T, and 180E, H2 comprises amino acid substitutions 143E, 145T, and 228D, and L2 comprises amino acid substitutions 121K, 124R, and 178R;
i) H1 comprises amino acid substitutions 125R and 186R, L1 comprises amino acid substitutions 122D, 129T, and 180E, H2 comprises amino acid substitutions 145T, 179E, and 228D, and L2 comprises amino acid substitutions 121K and 131K;
j) H1 comprises amino acid substitutions 125R and 186R, L1 comprises amino acid substitutions 122D, 129T, and 180E, H2 comprises amino acid substitutions 145T, 179E, and 228D, and L2 comprises amino acid substitutions 121K, 129K, and 131K;
k) H1 comprises amino acid substitutions 125R and 179K, L1 comprises amino acid substitutions 122D, 129T, and 180E, H2 comprises amino acid substitutions 143E and 145T, and L2 comprises amino acid substitutions 124R, 135W, 160K, and 178R;
l) H1 comprises amino acid substitutions 125R and 179K, L1 comprises amino acid substitutions 122D, 129T, and 180E, H2 comprises amino acid substitutions 143E and 145T, and L2 comprises amino acid substitutions 124R, 135W, and 178R;
m) H1 comprises amino acid substitutions 125R and 179K, L1 comprises amino acid substitutions 122D, 129T, and 180E, H2 comprises amino acid substitutions 143E, 145T, and 228D, and L2 comprises amino acid substitutions 121K, 124R, 160K, and 178R;
n) H1 comprises amino acid substitutions 125R and 179K, L1 comprises amino acid substitutions 122D, 129T, and 180E, H2 comprises amino acid substitutions 143E, 145T, and 228D, and L2 comprises amino acid substitutions 121K, 124R, and 178R;
o) H1 comprises amino acid substitutions 125R and 186R, L1 comprises amino acid substitutions 122D, 129T, and 180E, H2 comprises amino acid substitutions 143E, 145T, and 179E, and L2 comprises amino acid substitutions 124R, 135W, 160K, and 178R;
p) H1 comprises amino acid substitutions 125R and 179K, L1 comprises amino acid substitutions 122D, 129T, and 180E, H2 comprises amino acid substitutions 143E, 145T, 179E, and 228D, and L2 comprises amino acid substitutions 121K, 124R, and 178R;
q) H1 comprises amino acid substitutions 125R and 186R, L1 comprises amino acid substitutions 122D, 129T, and 180E, H2 comprises amino acid substitutions 143E, 145T, 179E, and 228D, and L2 comprises amino acid substitutions 121K, 124R, 160K, and 178R;
r) H1 comprises amino acid substitutions 125R and 186R, L1 comprises amino acid substitutions 122D, 129T, and 180E, H2 comprises amino acid substitutions 143E, 145T, 179E, and 228D, and L2 comprises amino acid substitutions 121K, 124R, and 178R;
s) H1 comprises amino acid substitutions 125R and 188K, L1 comprises amino acid substitutions 122D, 129T, 176E, and 178E, H2 comprises amino acid substitutions 143E, 145T, 179E, and 228D, and L2 comprises amino acid substitutions 121K, 124R, and 178R; or
t) H1 comprises amino acid substitutions 125R and 179K, L1 comprises amino acid substitutions 122D, 129T, and 180E, H2 comprises amino acid substitutions 145T, 179E, and 228D, and L2 comprises amino acid substitutions 121K and 131K.

46. The construct of claim 45, wherein the amino acid substitutions promote preferential pairing of H1 with L1 as compared to L2, and/or that promote preferential pairing of H2 with L2 as compared to L1, when H1, H2, L1 and L2 are co-expressed in a cell or a mammalian cell, or when H1, H2, L1 and L2 are co-expressed in a cell-free expression system, or when H1 and L1 are produced in a cell and H2 and L2 are produced in a different cell and the products of the two cells are mixed via a redox production method, or when H1 and L1 are produced in a cell-free expression system and H2 and L2 are produced in a different cell-free expression system and the products of the two cell-free expression systems are mixed.

47. The construct of claim 46, wherein the sequences of each of H1, H2, L1, and L2 are derived from human sequences or humanized sequences.

48. The construct of claim 47, wherein the construct further comprises a dimeric Fc having two Fc polypeptides each comprising a CH2 domain sequence and a CH3 domain sequence, and wherein one Fc polypeptide is coupled with or without linkers to the first Fab region and the other Fc polypeptide is coupled with or without linkers to second Fab region.

49. The construct of claim 48, wherein the Fc is a human Fc.

50. The construct of claim 49, wherein the human Fc comprises one or more substitutions as compared to wild type in at least one of the CH3 domain sequences that promote preferential pairing between heterodimeric CH3 domain sequences relative to homodimeric CH3 domain sequences.

51. The construct of claim 50, wherein the human Fc comprises:
i) a heterodimeric IgG1 Fc having the substitutions L351Y_F405A_Y407V in the first Fc polypeptide, and the substitutions T366L_K392M_T394W in the second Fc polypeptide;
ii) a heterodimeric IgG1 Fc having the substitutions L351Y_F405A_Y407V in the first Fc polypeptide, and the substitutions T366L_K392L_T394W in the second Fc polypeptide;
iii) a heterodimeric IgG1 Fc having the substitutions T350V_L351Y_F405A_Y407V in the first Fc polypeptide, and the substitutions T350V_T366L_K392L_T394W in the second Fc polypeptide;
iv) a heterodimeric IgG1 Fc having the substitutions T350V_L351Y_F405A_Y407V in the first Fc polypeptide, and the substitutions T350V_T366L_K392M_T394W in the second Fc polypeptide; or
v) a heterodimeric IgG1 Fc having the substitutions T350V_L351Y_S400E_F405A_Y407V in the first Fc polypeptide, and the substitutions T350V_T366L_N390R_K392M_T394W in the second Fc polypeptide,
wherein the numbering of amino acid residues in the Fc is according to EU numbering.

52. The construct of claim 48, wherein the Fc comprises one or more modifications to promote selective binding of Fc-gamma receptors, to reduce or eliminate binding to Fc-gamma receptors, or to promote binding to FcRn.

53. The construct of claim 45, wherein the one or more IgG hinge regions comprise one or more IgG1 hinge regions.

54. The construct of claim 45, conjugated to a therapeutic agent.

55. A polynucleotide or set of polynucleotides that encodes the construct of claim 45.

56. A vector or set of vectors comprising one or more of the polynucleotides or sets of polynucleotides of claim 55.

57. An isolated cell comprising the polynucleotide or set of polynucleotides of claim 55.

58. A pharmaceutical composition comprising the antigen-binding polypeptide construct of claim 45 and a pharmaceutically acceptable carrier.

59. A method of preparing the construct according to claim 45, comprising the steps of:
(a) obtaining a host cell comprising a polynucleotide or set of polynucleotides encoding the antigen-binding polypeptide construct;
(b) culturing the host cell in a host cell culture under conditions that allow expression of the antigen-binding polypeptide construct, and
(c) collecting the antigen-binding polypeptide construct from the host cell culture.

60. The construct of claim 45, wherein:
H1 comprises amino acid substitutions 125R and 188K, L1 comprises amino acid substitutions 122D, 129T, 176E, and 178E, H2 comprises amino acid substitutions 143E, 145T, 179E, and 228D, and L2 comprises amino acid substitutions 121K, 124R, and 178R.

61. The construct of claim 60, wherein the amino acid substitutions promote preferential pairing of H1 with L1 as compared to L2, and/or that promote preferential pairing of H2 with L2 as compared to L1, when H1, H2, L1 and L2 are co-expressed in a cell or a mammalian cell, or when H1, H2, L1 and L2 are co-expressed in a cell-free expression system, or when H1 and L1 are produced in a cell and H2 and L2 are produced in a different cell and the products of the two cells are mixed via a redox production method, or when H1 and L1 are produced in a cell-free expression system and H2 and L2 are produced in a different cell-free expression system and the products of the two cell-free expression systems are mixed.

62. The construct of claim 61, wherein the sequences of each of H1, H2, L1, and L2 are derived from human sequences or humanized sequences.

63. The construct of claim 60, wherein the construct further comprises a dimeric Fc having two Fc polypeptides each comprising a CH2 domain sequence and a CH3 domain sequence, and wherein one Fc polypeptide is coupled with or without linkers to the first Fab region and the other Fc polypeptide is coupled with or without linkers to second Fab region.

64. The construct of claim 63, wherein the Fc is a human Fc.

65. The construct of claim 64, wherein the human Fc comprises one or more substitutions as compared to wild type in at least one of the CH3 domain sequences that promote preferential pairing between heterodimeric CH3 domain sequences relative to homodimeric CH3 domain sequences.

66. The construct of claim 65, wherein the human Fc comprises:
i) a heterodimeric IgG1 Fc having the substitutions L351Y_F405A_Y407V in the first Fc polypeptide, and the substitutions T366L_K392M_T394W in the second Fc polypeptide;
ii) a heterodimeric IgG1 Fc having the substitutions L351Y_F405A_Y407V in the first Fc polypeptide, and the substitutions T366L_K392L_T394W in the second Fc polypeptide;
iii) a heterodimeric IgG1 Fc having the substitutions T350V_L351Y_F405A_Y407V in the first Fc polypeptide, and the substitutions T350V_T366L_K392L_T394W in the second Fc polypeptide;
iv) a heterodimeric IgG1 Fc having the substitutions T350V_L351Y_F405A_Y407V in the first Fc polypeptide, and the substitutions T350V_T366L_K392M_T394W in the second Fc polypeptide; or
v) a heterodimeric IgG1 Fc having the substitutions T350V_L351Y_S400E_F405A_Y407V in the first Fc polypeptide, and the substitutions T350V_T366L_N390R_K392M_T394W in the second Fc polypeptide,
wherein the numbering of amino acid residues in the Fc is according to EU numbering.

67. The construct of claim 63, wherein the Fc comprises one or more modifications to promote selective binding of Fc-gamma receptors, to reduce or eliminate binding to Fc-gamma receptors, or to promote binding to FcRn.

68. The construct of claim 63, conjugated to a therapeutic agent.
